(19) United States
(12) Patent Application Publication
(10) Pub. No.: US 2024/0218021 A1
(43) Pub. Date: Jul. 4, 2024

(54) CYCLIN INHIBITORS

(71) Applicant: CIRCLE PHARMA, INC., South San Francisco, CA (US)

(72) Inventors: Andrew T. Bockus, South San Francisco, CA (US); Sik Fai Siegfried Leung, South San Francisco, CA (US); David J. Earp, South San Francisco, CA (US); Pablo Santiago Garcia Dominguez, South San Francisco, CA (US); David C. Spellmeyer, South San Francisco, CA (US); Luis Hernandez, South San Francisco, CA (US); Miguel Paolo Baldomero, South San Francisco, CA (US); Catherine E. Gleason, South San Francisco, CA (US); Breena F. Walton, South San Francisco, CA (US); Rajinder Singh, South San Francisco, CA (US); James B. Aggen, South San Francisco, CA (US); Nathan J. Dupper, South San Francisco, CA (US); Justin A. Shapiro, South San Francisco, CA (US); Constantine Kreatsoulas, South San Francisco, CA (US); Ramesh B. Bambal, South San Francisco, CA (US); Chat Cheong Gabriel Fung, South San Francisco, CA (US); Mahesh Ramaseshan, South San Francisco, CA (US)

(21) Appl. No.: 18/491,380

(22) Filed: Oct. 20, 2023

Related U.S. Application Data
(60) Provisional application No. 63/380,562, filed on Oct. 21, 2022.

(51) Int. Cl. C07K 7/56; A61P 35/00

(52) U.S. Cl. CPC C07K 7/56; A61P 35/00; A61K 38/00

(57) ABSTRACT

Disclosed herein are compounds of Formula I and methods for making the same:

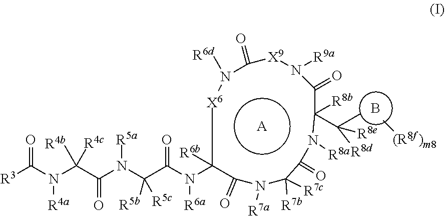

Also described herein are the use of such compounds, compositions for the treatment of diseases and disorders that are mediated, at least in part, by one or more cyclins, including cancer, and intermediates useful in the preparation of these compounds.

### CROSS-REFERENCES TO RELATED APPLICATIONS

This application claims priority to U.S. Provisional Application No. 63/380,562, filed Oct. 21, 2022, which is incorporated herein in its entirety for all purposes.

### BACKGROUND

Cyclins are a family of proteins that play a central role in the regulation of the cell cycle. Specific cyclins, including Cyclins D, E, A and B, are expressed at the different stages of the cell cycle, during which they bind and activate their cognate cyclin dependent kinases (CDKs), including CDKs 1, 2, 4 and 6, to form cyclin-CDK complexes that orchestrate progression and transitions through the different stages of the cell cycle. Disruptions of the normal regulatory functions of cyclin-CDK complexes are common drivers of oncogenesis and the rapid proliferation of cancer cells. The central role of cyclins and CDKs in the cell cycle makes these proteins and their complexes attractive targets for treating proliferative disorders and cancer. To date, most inhibitors of cyclin-CDK complexes target the kinase activity of CDKs (“CDK inhibitors”) and include therapeutics both in development and approved for clinical use. Alternative approaches could include disrupting the association of cyclins with CDKs or the interaction of a particular cyclin-CDK complex with its substrates or regulators.

Although CDK inhibitors have been developed and proven successful in certain cancers, they are currently limited by their relative lack of selectivity, small therapeutic window, and ultimately the development of resistance. As such, there is a need to develop agents that offer alternative approaches to inhibiting the function of cyclin-CDK complexes as a means to modulate the cell cycle. Such agents could provide new tools in the treatment of proliferative diseases. The present disclosure addresses this need by providing compounds that inhibit the binding of substrates to various cyclins, thereby disrupting the function of cyclin-CDK complexes.

### BRIEF SUMMARY

In one embodiment, provided herein is a compound of Formula (I):

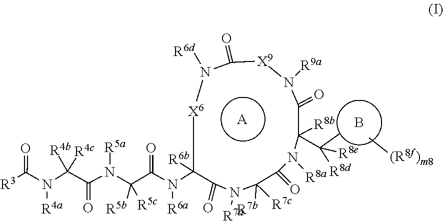

wherein
R³ is
(a) C₁₋₈ alkyl, C₂₋₈ alkenyl, C₂₋₈ alkynyl, or C₁₋₈ haloalkyl, each substituted with 0 to 5 R^{3a};
(b) C₃₋₁₂ cycloalkyl substituted with 0 to 5 R^{3b}; or
(c) heterocycloalkyl having 3 to 6 ring members and 1 to 3 heteroatoms each independently N, O, or S, wherein the heterocycloalkyl is substituted with 0 to 5 R^{3c};
each R^{3a} is independently —OH, C₁₋₃ alkoxy, —O—(CH₂CH₂O)₁₋₄—C₁₋₄ alkyl, —O—(CH₂CH₂O)₁₋₄-heterocycloalkyl, C₁₋₃ haloalkoxy, —NR^{3a1}R^{3a2}, —O—C(O)C₁₋₄ alkyl, C₃₋₆ cycloalkyl, phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S;
each R^{3b} is independently C₁₋₄ alkyl, C₂₋₄ alkenyl, C₂₋₄ alkynyl, halo, C₁₋₄ haloalkyl, cyano, —OH, C₁₋₃ alkoxy, C₁₋₃ haloalkoxy, —NR^{3b1}R^{3b2}, —N(R^{3b3})C(O)R^{3b4}, phenyl, or heteroaryl having 5 to 6 ring members and 1 to 3 heteroatoms each independently N, O or S;
each R^{3c} is independently C₁₋₄ alkyl, C₁₋₄ haloalkyl, oxo, or C₃₋₆ cycloalkyl;
each R^{3a1}, R^{3a2}, R^{3b1}, R^{3b2}, and R^{3b3} is independently H or C₁₋₄ alkyl;
each R^{3b4} is C₁₋₄ alkyl, or C₁₋₄ haloalkyl;
R^{4a} is H or C₁₋₄ alkyl;
R^{4b} and R^{4c} are each independently H, C₁₋₈ alkyl, C₁₋₈ alkyl-OH, —NR^{4c1}R^{4c2}, —C₁₋₄ alkyl-NR^{4c1}R^{4c2}, C₃₋₆ cycloalkyl, C₁₋₄ alkyl-C₃₋₆ cycloalkyl, heterocycloalkyl, —C₁₋₄ alkyl-heterocycloalkyl, heteroaryl, or C₁₋₄ alkyl-heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O, or S;
alternatively, R^{4c} and R^{4a} together with the carbon and nitrogen to which each is attached combine to form a heterocycloalkyl having 4 to 6 ring members and 0 to 2 additional heteroatoms each independently N, O or S, wherein the heterocycloalkyl is substituted with 0 to 2 R^{4a1};
each R^{4c1} and R^{4c2} are independently C₁₋₄ alkyl or C₂₋₆ alkoxyalkyl;
each R^{4a1} is independently C₁₋₄ alkyl, —OH, C₁₋₄ alkyl-OH, C₁₋₄ alkoxy, halo, or —N(R^{4a2})S(O)₂—C₁₋₄ alkyl;
R^{4a2} is H or C₁₋₄ alkyl;
alternatively, two R^{4a1} groups on adjacent ring atoms combine to form a phenyl ring substituted with 0 to 2 R^{4a3};
each R^{4a3} is independently C₁₋₄ alkyl, —OH, C₁₋₄ alkyl-OH, C₁₋₄ alkoxy, or halo;
R^{5a} is H or C₁₋₄ alkyl;
R^{5b} and R^{5c} are each independently H, C₁₋₈ alkyl, C₁₋₈ alkyl-OH, C₂₋₆ alkoxyalkyl, C₁₋₈ haloalkyl, —C₁₋₄ alkyl-NR^{5b1}R^{5b2}, —C₁₋₃ alkyl-C(O)NR^{5b1}R^{5b2}, C₁₋₄ alkyl-N(R^{5b3})C(O)R^{5b4}, C₃₋₆ cycloalkyl, C₁₋₄ alkyl-C₃₋₆ cycloalkyl, heteroaryl, or C₁₋₄ alkyl-heteroaryl, wherein each heteroaryl has 5 to 6 ring members and 1 to 3 heteroatoms each independently N, O, or S, and wherein each cycloalkyl and heteroaryl is substituted with 0 to 3 R^{5b5};
each R^{5b1} and R^{5b2} are independently H, C₁₋₄ alkyl, C₁₋₄ haloalkyl, —C(O)C₁₋₄ alkyl, or —C(O)C₁₋₄ haloalkyl;
alternatively, R^{5b1} and R^{5b2} on the same nitrogen atom combine to form a heterocycloalkyl having 4 to 6 ring members and 0 to 2 additional heteroatoms each independently N, O or S, wherein the heterocycloalkyl is substituted with 0 to 3 R^{5b5};
each R^{5b3} is H or C₁₋₄ alkyl;
each R^{5b4} is a heteroaryl having 5 to 6 ring members and 1 to 3 heteroatoms each independently N, O or S substituted with 0 to 3 R^{5b5};
each R^{5b5} is independently C₁₋₄ alkyl, halo, C₁₋₄ haloalkyl, —NH₂, —N(C₁₋₄ alkyl)₂, or NH(C₁₋₄ alkyl);
X⁶ is C₂₋₅ alkylene;
R^{6a} is H, C₁₋₄ alkyl, C₁₋₄ deuteroalkyl, C₂₋₆ alkoxyalkyl, C₃₋₆ cycloalkyl, C₁₋₄ alkyl-C₃₋₆ cycloalkyl, heterocycloalkyl or C₁₋₄ alkyl-heterocycloalkyl, wherein the heterocycloalkyl has 4 to 6 ring members and 1 to 3 heteroatoms each independently N, O, or S;
R^{6b} is H or C₁₋₆ alkyl;
R^{6d} is H, C₁₋₄ alkyl, C₁₋₄ deuteroalkyl, —OH, or C₂₋₆ alkoxyalkyl;
R^{7a} is H or C₁₋₄ alkyl;
R^{7b} and R^{7c} are each independently H, C₁₋₈ alkyl, C₃₋₆ cycloalkyl, or C₁₋₄ alkyl-C₃₋₆ cycloalkyl;
R^{8a} is H, C₁₋₄ alkyl, C₁₋₄ deuteroalkyl, C₂₋₆ alkoxyalkyl, C₃₋₆ cycloalkyl or —C₁₋₄ alkyl-C₃₋₆ cycloalkyl;
R^{8b}, R^{8d}, and R^{8e} are each independently H or C₁₋₄ alkyl;
alternatively R^{8b} and R^{8d} together with the carbons to which each is attached combine to form a C₃₋₆ cycloalkyl;
ring B is phenyl or heteroaryl having 5 to 12 ring members and 1 to 6 heteroatoms each independently N, O or S;
the subscript m8 is an integer from 0 to 5;
each R^{8f} is independently C₁₋₄ alkyl, C₂₋₄ alkenyl, C₂₋₄ alkynyl, C₁₋₄ alkoxy, C₂₋₈ alkoxyalkyl, halo, C₁₋₄ haloalkyl, C₁₋₄ haloalkoxy, cyano, —NR^{8f1}R^{8f2}, —C(O)NR^{8f1}R^{8f2}, —N(R^{8f1})C(O)R^{8f2}, C₃₋₆ cycloalkyl, —O—C₃₋₆ cycloalkyl, C₁₋₄ alkyl-C₃₋₆ cycloalkyl, —O—C₁₋₄ alkyl-C₃₋₆ cycloalkyl, heterocycloalkyl, C₁₋₄ alkyl-heterocycloalkyl, phenyl, —O-phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S, wherein each cycloalkyl, heterocycloalkyl, phenyl, and heteroaryl is substituted with 0 to 3 R^{8f3};
each R^{8f1} and R^{8f2} are independently H or C₁₋₄ alkyl;
each R^{8f3} is independently C₁₋₄ alkyl, —OH, C₁₋₄ alkoxy, —SH, —S—C₁₋₄ alkyl, halo, C₁₋₄ haloalkyl, C₁₋₄ haloalkoxy, —C(O)C₁₋₄ alkyl, —O—C₃₋₆ cycloalkyl, —O—C₁₋₄ alkyl-C₃₋₆ cycloalkyl, or heterocycloalkyl having 4 to 6 members and 0 to 2 additional heteroatoms each independently N, O or S;
X⁹ is C₁₋₃ alkylene substituted with R^{9b} and R^{9c};
R^{9a} is H or C₁₋₄ alkyl;
R^{9b} and R^{9c} are each independently H, C₁₋₆ alkyl, C₁₋₆ haloalkyl, C₁₋₆ alkyl-OH, C₂₋₆ alkoxyalkyl, C₃₋₆ cycloalkyl, C₁₋₄ alkyl-C₃₋₆ cycloalkyl, heteroaryl, or C₁₋₄ alkyl-heteroaryl, wherein each heteroaryl has 5 to 6 ring members and from 1 to 3 heteroatoms each independently N, O, or S, and each cycloalkyl and heteroaryl is independently substituted with 0 to 3 R^{9c1};
alternatively, R^{9b} and R^{9c} together with the carbon to which each is attached combine to form a C₃₋₄ cycloalkyl substituted with 0 to 2 R^{9c2}; or
alternatively, R^{9c} and R^{9a} together with the carbon and nitrogen to which each is attached combine to form a heterocycloalkyl having 4 to 6 members and 0 to 2 additional heteroatoms each independently N, O or S, wherein the heterocycloalkyl is substituted with 0 to 2 R^{9c2};
each R^{9c1} and R^{9c2} is independently C₁₋₄ alkyl, —OH, C₁₋₄ alkoxy, halo, C₁₋₄ haloalkyl, or C₁₋₄ haloalkoxy; and
ring A comprises 15 to 17 ring atoms;
or a pharmaceutically acceptable salt thereof.

In another embodiment, the present invention provides a pharmaceutical composition comprising a compound of the present invention, and a pharmaceutically acceptable excipient.

In another embodiment, the present invention provides a method of treating a disease or disorder mediated at least in part by cyclin activity, the method comprising administering to a subject in need thereof, a therapeutically effective amount of a compound of the present invention, or a pharmaceutically acceptable salt thereof, or a pharmaceutical composition of the present invention, thereby treating the disorder or condition.

In another embodiment, the present invention provides a method of treating a cancer mediated at least in part by cyclin A, the method comprising administering to a subject in need thereof, a therapeutically effective amount of a compound of the present invention, or a pharmaceutically acceptable salt thereof, or a pharmaceutical composition of the present invention, thereby treating the cancer.

In another embodiment, the present invention provides intermediates useful in the preparation of compounds of Formula (I).

Other objects, features, and advantages of the present disclosure will be apparent to one of skill in the art from the following detailed description and figures.

### BRIEF DESCRIPTION OF THE DRAWINGS

FIGS. 1A and 1B shows western blots from H1048 cell lysates following treatment with Example 458 compared to its enantiomer Example 680 showing displacement of two substrates, E2F1 (1A) and CDC6 (1B) from their complex with Cyclin A2 only by the active Example.

FIGS. 2A and 2B shows that IV administration of an exemplary compound in this application (Example 456) causes tumor regression in an in vivo SCLC model (tumor volume plot, 2A) at tolerated dose levels (body weight change plot, 2B).

### DETAILED DESCRIPTION

### I. General

Provided herein are compounds and compositions that disrupt the typical cellular function of cyclins. Also provided herein are, for example, methods of treating or preventing a disease, disorder or condition, or a symptom thereof, mediated by cyclin activity.

Complexes between cyclins and cyclin dependent kinases (CDKs) are responsible for phosphorylating a wide range of substrates, thereby modulating the activity of the substrates. Many of these substrates are important in the cell cycle and the cyclin and CDKs that regulate these substrates therefore play key roles in regulating the cell cycle, including Cyclins D, A, E and B, and CDKs 1, 2, 4 and 6. Without being bound to any particular theory, certain substrates, including p21, p27, Rb, E2F and CDC6, first bind to the cyclin-CDK complex via a conserved RxL motif within the substrate (Adams et al. Mol Cell Biol. 1996. 16(12):6223-33) and bind to a region with the cyclin that is referred to as an RxL binding domain or a “hydrophobic patch” (Brown et al. Nat Cell Biol. 1999. 1(7):438-43) and contains a highly conserved MRAIL motif. Compounds that disrupt the binding of substrates to cyclins have been posited to be of potential therapeutic utility, including in the disruption of cancer cell proliferation (Chen et al. Proc Natl Acad Sci USA. 1999. 96(8):4325-9).

Without being bound to any particular theory, it is believed that compounds of the present disclosure inhibit the binding of substrates to the hydrophobic patch region of cyclins including, but not limited to, Cyclins A, E and B. Compounds of the present disclosure include compounds that bind more potently to one or more cyclins.

### II. Definitions

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be substituted or unsubstituted.

“Alkoxyalkyl” refers to alkyl group connected to an oxygen atom that is further connected to an second alkyl group, the second alkyl group being the point of attachment to the remainder of the molecule: alkyl-O-alkyl. The alkyl portion can have any suitable number of carbon atoms, such as C₂₋₆. Alkoxyalkyl groups include, for example, methoxymethyl, ethoxymethyl, methoxyethyl, ethoxyethyl, etc. The alkoxy groups can be substituted or unsubstituted.

“Halo” or “halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2-trifluoroethoxy, perfluoroethoxy, etc.

“Cycloalkyl” refers to a saturated or partially unsaturated, monocyclic, spirocyclic, fused or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobuteneyl, cyclopenteneyl, cyclohexeneyl, cyclohexadieneyl (1,3- and 1,4-isomers), cyclohepteneyl, cycloheptadieneyl, cycloocteneyl, cyclooctadieneyl (1,3-, 1,4- and 1,5-isomers), norborneneyl, and norbornadieneyl. When cycloalkyl is a C₃₋₆ monocyclic cycloalkyl, exemplary groups include, but are not limited to cyclopropyl, cyclobutyl, cyclopentyl, cyclohexyl, cyclohexeneyl, cyclohexadieneyl (1,3- and 1,4-isomers). When cycloalkyl is a C₅₋₁₀ fused bicyclic cycloalkyl, exemplary groups include, but are not limited to bicyclo[3.1.0]hexanyl, bicyclo[4.1.0]heptanyl, bicyclo[4.2.0]octanyl, and octahydro-1H-indenyl. When cycloalkyl is a C₅₋₁₀ bridged polycyclic cycloalkyl, exemplary groups include, but are not limited to bicyclo[2.2.1]heptane, bicyclo[3.1.1]heptane, and bicyclo[2.1.1]hexane. When cycloalkyl is a C₅₋₁₀ spirocycloalkyl, exemplary groups include, but are not limited to spiro[3.3]heptane, spiro[3.4]octane, spiro[3.5]nonanyl, spiro[2.5]octane, and spiro[2.4]heptane. Cycloalkyl groups can be substituted or unsubstituted.

“Heterocycloalkyl” refers to a saturated or partially unsaturated, monocyclic, spirocyclic, fused or bridged polycyclic ring assembly having from 3 to 12 ring members and from 1 to 4 heteroatoms of N, O and S. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), tetrahydropyridine, oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. Heterocycloalkyl groups can be unsubstituted or substituted.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl is a monocyclic heterocycloalkyl having 3 to 6 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocycloalkyl can also be monocyclic heterocycloalkyl having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 12 ring atoms, where from 1 to 6 of the ring atoms are a heteroatom such as N, O or S. The heteroatoms can also be oxidized, such as, but not limited to, —S(O)— and —S(O)₂—. Heteroaryl groups can include any number of ring atoms, such as, 5 to 6, 5 to 8, 5 to 9, 5 to 10, 5 to 12, or 9 to 12 ring members. Any suitable number of heteroatoms can be included in the heteroaryl groups, such as 1, 2, 3, 4, 5, or 6, or 1 to 2, 1 to 3, 1 to 4, 1 to 5, 2 to 3, 2 to 4, 2 to 5, 2 to 6, 3 to 4, 3 to 5, or 3 to 6. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

“Oxo” refers to an oxygen atom connected to the point of attachment by a double bond (═O).

“Pharmaceutically acceptable excipient” refers to a substance that aids the formulation and/or administration of an active agent to a subject. Pharmaceutical excipients useful in the present disclosure include, but are not limited to, binders, fillers, disintegrants, lubricants, coatings, sweeteners, flavors and colors. One of skill in the art will recognize that other pharmaceutical excipients are useful in the present disclosure.

“Subject” refers to animals such as mammals, including, but not limited to, primates (e.g., humans), cows, sheep, goats, horses, dogs, cats, rabbits, rats, mice and the like. In some embodiments, the subject is a human.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Therapeutically effective amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins)

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition. The treatment or amelioration of symptoms can be based on any objective or subjective parameter; including, e.g., the result of a physical examination.

### III. Compounds

In some embodiments, the present invention provides a compound of Formula (I):

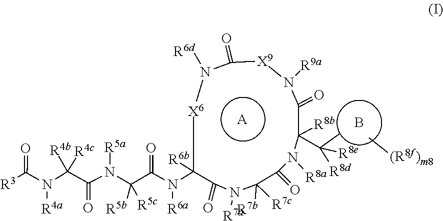

wherein
R³ is
(a) C₁₋₈ alkyl, C₂₋₈ alkenyl, C₂₋₈ alkynyl, or C₁₋₈ haloalkyl, each substituted with 0 to 5 R^{3a};
(b) C₃₋₁₂ cycloalkyl substituted with 0 to 5 R^{3b}; or
(c) heterocycloalkyl having 3 to 6 ring members and 1 to 3 heteroatoms each independently N, O, or S, wherein the heterocycloalkyl is substituted with 0 to 5 R^{3c};
each R^{3a} is independently —OH, C₁₋₃ alkoxy, —O—(CH₂CH₂O)₁₋₄—C₁₋₄ alkyl, —O—(CH₂CH₂O)₁₋₄-heterocycloalkyl, C₁₋₃ haloalkoxy, —NR^{3a1}R^{3a2}, —O—C(O)C₁₋₄ alkyl, C₃₋₆ cycloalkyl, phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S;
each R^{3b} is independently C₁₋₄ alkyl, C₂₋₄ alkenyl, C₂₋₄ alkynyl, halo, C₁₋₄ haloalkyl, cyano, —OH, C₁₋₃ alkoxy, C₁₋₃ haloalkoxy, —NR^{3b1}R^{3b2}, —N(R^{3b3})C(O)R^{3b4}, phenyl, or heteroaryl having 5 to 6 ring members and 1 to 3 heteroatoms each independently N, O or S;
each R^{3c} is independently C₁₋₄ alkyl, C₁₋₄ haloalkyl, oxo, or C₃₋₆ cycloalkyl;
each R^{3a1}, R^{3a2}, R^{3b1}, R^{3b2}, and R^{3b3} is independently H or C₁₋₄ alkyl;
each R^{3b4} is C₁₋₄ alkyl or C₁₋₄ haloalkyl;
R^{4a} is H or C₁₋₄ alkyl;
R^{4b} and R^{4c} are each independently H, C₁₋₈ alkyl, C₁₋₈ alkyl-OH, —NR^{4c1}R^{4c2}, —C₁₋₄ alkyl-NR^{4c1}R^{4c2}, C₃₋₆ cycloalkyl, C₁₋₄ alkyl-C₃₋₆ cycloalkyl, heterocycloalkyl, —C₁₋₄ alkyl-heterocycloalkyl, heteroaryl, or C₁₋₄ alkyl-heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O, or S;
alternatively, R^{4c} and R^{4a} together with the carbon and nitrogen to which each is attached combine to form a heterocycloalkyl having 4 to 6 ring members and 0 to 2 additional heteroatoms each independently N, O or S, wherein the heterocycloalkyl is substituted with 0 to 2 R^{4a1};
each R^{4c1} and R^{4c2} are independently C₁₋₄ alkyl or C₂₋₆ alkoxyalkyl;
each R^{4a1} is independently C₁₋₄ alkyl, —OH, C₁₋₄ alkyl-OH, C₁₋₄ alkoxy, halo, or —N(R^{4a2})S(O)₂—C₁₋₄ alkyl;
R^{4a2} is H or C₁₋₄ alkyl;
alternatively, two R^{4a1} groups on adjacent ring atoms combine to form a phenyl ring substituted with 0 to 2 R^{4a3};
each R^{4a3} is independently C₁₋₄ alkyl, —OH, C₁₋₄ alkyl-OH, C₁₋₄ alkoxy, or halo;
R^{5a} is H or C₁₋₄ alkyl;
R^{5b} and R^{5c} are each independently H, C₁₋₈ alkyl, C₁₋₈ alkyl-OH, C₂₋₆ alkoxyalkyl, C₁₋₈ haloalkyl, —C₁₋₄ alkyl-NR^{5b1}R^{5b2}, —C₁₋₃ alkyl-C(O)NR^{5b1}R^{5b2}, C₁₋₄ alkyl-N(R^{5b3})C(O)R^{5b4}, C₃₋₆ cycloalkyl, C₁₋₄ alkyl-C₃₋₆ cycloalkyl, heteroaryl, or C₁₋₄ alkyl-heteroaryl, wherein each heteroaryl has 5 to 6 ring members and 1 to 3 heteroatoms each independently N, O, or S, and wherein each cycloalkyl and heteroaryl is substituted with 0 to 3 R^{5b5};
each R^{5b1} and R^{5b2} are independently H, C₁₋₄ alkyl, C₁₋₄ haloalkyl, —C(O)C₁₋₄ alkyl, or —C(O)C₁₋₄ haloalkyl;
alternatively, R^{5b1} and R^{5b2} on the same nitrogen atom combine to form a heterocycloalkyl having 4 to 6 ring members and 0 to 2 additional heteroatoms each independently N, O or S, wherein the heterocycloalkyl is substituted with 0 to 3 R^{5b5};
each R^{5b3} is H or C₁₋₄ alkyl;
each R^{5b4} is a heteroaryl having 5 to 6 ring members and 1 to 3 heteroatoms each independently N, O or S substituted with 0 to 3 R^{5b5};
each R^{5b5} is independently C₁₋₄ alkyl, halo, C₁₋₄ haloalkyl, —NH₂, —N(C₁₋₄alkyl)₂, or NH(C₁₋₄ alkyl);
X⁶ is C₂₋₅ alkylene;
R^{6a} is H, C₁₋₄ alkyl, C₁₋₄ deuteroalkyl, C₂₋₆ alkoxyalkyl, C₃₋₆ cycloalkyl, C₁₋₄ alkyl-C₃₋₆ cycloalkyl, heterocycloalkyl or C₁₋₄ alkyl-heterocycloalkyl, wherein the heterocycloalkyl has 4 to 6 ring members and 1 to 3 heteroatoms each independently N, O, or S;
R^{6b} is H or C₁₋₆ alkyl;
R^{6d} is H, C₁₋₄ alkyl, C₁₋₄ deuteroalkyl, —OH, or C₂₋₆ alkoxyalkyl;
R^{7a} is H or C₁₋₄ alkyl;
R^{7b} and R^{7c} are each independently H, C₁₋₈ alkyl, C₃₋₆ cycloalkyl, or C₁₋₄ alkyl-C₃₋₆ cycloalkyl;
R^{8a} is H, C₁₋₄ alkyl, C₁₋₄ deuteroalkyl, C₂₋₆ alkoxyalkyl, C₃₋₆ cycloalkyl or —C₁₋₄ alkyl-C₃₋₆ cycloalkyl;
R^{8b}, R^{8d}, and R^{8e} are each independently H or C₁₋₄ alkyl;
alternatively R^{8b} and R^{8d} together with the carbons to which each is attached combine to form a C₃₋₆ cycloalkyl;
ring B is phenyl or heteroaryl having 5 to 12 ring members and 1 to 6 heteroatoms each independently N, O or S;
the subscript m8 is an integer from 0 to 5;
each R^{8f} is independently C₁₋₄ alkyl, C₂₋₄ alkenyl, C₂₋₄ alkynyl, C₁₋₄ alkoxy, C₂₋₈ alkoxyalkyl, halo, C₁₋₄ haloalkyl, C₁₋₄ haloalkoxy, cyano, —NR^{8f1}R^{8f2}, —C(O)NR^{8f1}R^{8f2}, —N(R^{8f1})C(O)R^{8f2}, C₃₋₆ cycloalkyl, —O—C₃₋₆ cycloalkyl, C₁₋₄ alkyl-C₃₋₆ cycloalkyl, —O—C₁₋₄ alkyl-C₃₋₆ cycloalkyl, heterocycloalkyl, C₁₋₄ alkyl-heterocycloalkyl, phenyl, —O-phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S, wherein each cycloalkyl, heterocycloalkyl, phenyl, and heteroaryl is substituted with 0 to 3 R^{8f3};
each R^{8f1} and R^{8f2} are independently H or C₁₋₄ alkyl;
each R^{8f3} is independently C₁₋₄ alkyl, —OH, C₁₋₄ alkoxy, —SH, —S—C₁₋₄ alkyl, halo, C₁₋₄ haloalkyl, C₁₋₄ haloalkoxy, —C(O)C₁₋₄ alkyl, —O—C₃₋₆ cycloalkyl, —O—C₁₋₄ alkyl-C₃₋₆ cycloalkyl, or heterocycloalkyl having 4 to 6 members and 0 to 2 additional heteroatoms each independently N, O or S;
X⁹ is C₁₋₃ alkylene substituted with R^{9b} and R^{9c};
R^{9a} is H or C₁₋₄ alkyl;
R^{9b} and R^{9c} are each independently H, C₁₋₆ alkyl, C₁₋₆ haloalkyl, C₁₋₆ alkyl-OH, C₂₋₆ alkoxyalkyl, C₃₋₆ cycloalkyl, C₁₋₄ alkyl-C₃₋₆ cycloalkyl, heteroaryl, or C₁₋₄ alkyl-heteroaryl, wherein each heteroaryl has 5 to 6 ring members and from 1 to 3 heteroatoms each independently N, O, or S, and each cycloalkyl and heteroaryl is independently substituted with 0 to 3 R^{9c1};
alternatively, R^{9b} and R^{9c} together with the carbon to which each is attached combine to form a C₃₋₄ cycloalkyl substituted with 0 to 2 R^{9c2}; or
alternatively, R^{9c} and R^{9a} together with the carbon and nitrogen to which each is attached combine to form a heterocycloalkyl having 4 to 6 members and 0 to 2 additional heteroatoms each independently N, O or S, wherein the heterocycloalkyl is substituted with 0 to 2 R^{9c2};
each R^{9c1} and R^{9c2} is independently C₁₋₄ alkyl, —OH, C₁₋₄ alkoxy, halo, C₁₋₄ haloalkyl, or C₁₋₄ haloalkoxy; and
ring A comprises 15 to 17 ring atoms;
or a pharmaceutically acceptable salt thereof.

In some embodiments, the compound, or the pharmaceutically acceptable salt thereof, is the compound of Formula (I) wherein ring A comprises 13 to 19 ring atoms. In some embodiments, the compound, or the pharmaceutically acceptable salt thereof, is the compound of Formula (I) wherein ring A comprises 15 to 17 ring atoms. In some embodiments, the compound, or the pharmaceutically acceptable salt thereof, is the compound of Formula (I) wherein ring A comprises 15 ring atoms. In some embodiment ring A comprises 16 ring atoms. In some embodiments, the compound, or the pharmaceutically acceptable salt thereof, is the compound of Formula (I) wherein ring A comprises 17 ring atoms.

### Residue 3

In some embodiments, the compound, or the pharmaceutically acceptable salt thereof, is the compound of Formula (I), (Ia), (Ia1), (Ib), (Ib1), (Ic), or (Ic1) wherein
R³ is
(a) C₁₋₆ alkyl, C₂₋₆ alkynyl, or C₁₋₆ haloalkyl, each substituted with 0 to 5 R^{3a};
(b) C₃₋₁₂ cycloalkyl substituted with 0 to 5 R^{3b}; or
(c) heterocycloalkyl having 3 to 6 ring members and 1 to 3 heteroatoms each independently N, O, or S, wherein the heterocycloalkyl is substituted with 0 to 5 R^{3c};
each R^{3a} is independently —OH, C₁₋₃ alkoxy, —O—(CH₂CH₂O)₁₋₃—C₁₋₄ alkyl, —O—(CH₂CH₂O)₁₋₂-heterocycloalkyl, C₁₋₃ haloalkoxy, —NR^{3a1}R^{3a2}, —C(O)C₁₋₄ alkyl, C₃₋₆ cycloalkyl, phenyl, or heteroaryl, wherein each heterocycloalkyl has 4 to 6 ring members and 1 to 3 heteroatoms each independently N, O, or S having 5 to 6 ring members and 1 to 3 heteroatoms each independently N, O or S;
each R^{3b} is independently C₁₋₄ alkyl, C₂₋₄ alkynyl, halo, C₁₋₄ haloalkyl, cyano, —N(R^{3b3})C(O)R^{3b4}, phenyl, or heteroaryl having 5 to 6 ring members and 1 to 3 heteroatoms each independently N, O or S;
each R^{3c} is independently C₁₋₄ alkyl, C₁₋₄ haloalkyl, oxo, or C₃₋₆ cycloalkyl;
each R^{3a1}, R^{3a2}, and R^{3b3} is independently H or C₁₋₄ alkyl; and
each R^{3b4} is C₁₋₄ alkyl.

In some embodiments, the compound, or the pharmaceutically acceptable salt thereof, is the compound of Formula (I), (Ia), (Ia1), (Ib), (Ib1), (Ic), or (Ic1) wherein
R³ is
(a) C₁₋₆ alkyl, C₂₋₆ alkynyl, or C₁₋₆ haloalkyl, each substituted with 0 to 5 R^{3a};
(b) C₃₋₁₂ cycloalkyl substituted with 0 to 5 R^{3b}; or
(c) heterocycloalkyl having 3 to 6 ring members and 1 to 3 heteroatoms each independently N, O, or S, wherein the heterocycloalkyl is substituted with 0 to 5 R^{3c};
each R^{3a} is independently —OH, C₁₋₃ alkoxy, —O—(CH₂CH₂O)₁₋₃—C₁₋₄ alkyl, —O—(CH₂CH₂O)₁₋₂-heterocycloalkyl, C₁₋₃ haloalkoxy, —NH₂, —O—C(O)C₁₋₄ alkyl, C₃₋₆ cycloalkyl, or phenyl, wherein each heterocycloalkyl has 4 to 6 ring members and 1 to 3 heteroatoms each independently N, O, or S;
each R^{3b} is independently C₁₋₄ alkyl, C₂₋₄ alkynyl, halo, C₁₋₄ haloalkyl, cyano, or —NHC(O)C₁₋₄ alkyl; and
each R^{3c} is independently C₁₋₄ alkyl, C₁₋₄ haloalkyl, or oxo.

In some embodiments, the compound, or the pharmaceutically acceptable salt thereof, is the compound of Formula (I), (Ia), (Ia1), (Ib), (Ib1), (Ic), or (Ic1) wherein R³ is (a) C₁₋₆ alkyl, C₂₋₆ alkynyl, or C₁₋₆ haloalkyl, each substituted with 0 to 5 R^{3a}. In some embodiments, the compound, or the pharmaceutically acceptable salt thereof, is the compound of Formula (I), (Ia), (Ia1), (Ib), (Ib1), (Ic), or (Ic1) wherein R³ is C₁₋₆ alkyl substituted with 0 to 5 R^{3a}. In some embodiments, the compound, or the pharmaceutically acceptable salt thereof, is the compound of Formula (I), (Ia), (Ia1), (Ib), (Ib1), (Ic), or (Ic1) wherein R³ is C₂₋₆ alkynyl substituted with 0 to 5 R^{3a}. In some embodiments, the compound, or the pharmaceutically acceptable salt thereof, is the compound of Formula (I), (Ia), (Ia1), (Ib), (Ib1), (Ic), or (Ic1) wherein R³ is C₁₋₆ haloalkyl, substituted with 0 to 5 R^{3a}. These embodiments of R³ can be combined with any of the embodiments described herein for R^{3a}.

In some embodiments, the compound, or the pharmaceutically acceptable salt thereof, is the compound of Formula (I), (Ia), (Ia1), (Ib), (Ib1), (Ic), or (Ic1) wherein R³ is substituted with 0 R^{3a} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 1 R^{3a} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 2 R^{3a} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 3 R^{3a} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 4 R^{3a} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 5 R^{3a} groups. These embodiments of R³ can be combined with any of the embodiments described herein for R^{3a}.

In some embodiments, the compound, or the pharmaceutically acceptable salt thereof, is the compound of Formula (I), (Ia), (Ia1), (Ib), (Ib1), (Ic), or (Ic1) wherein each R^{3a} is independently —OH, C₁₋₃ alkoxy, C₁₋₃ haloalkoxy, —NH₂, —O—C(O)C₁₋₄ alkyl, C₃₋₆ cycloalkyl, or phenyl. In some embodiments, the compound, or the pharmaceutically acceptable salt thereof, is the compound of Formula (I), (Ia), (Ia1), (Ib), (Ib1), (Ic), or (Ic1) wherein each R^{3a} is independently —OH, C₁₋₃ alkoxy, C₁₋₃ haloalkoxy, —NH₂, —O—C(O)C₁₋₄ alkyl, or C₃₋₆ cycloalkyl. In some embodiments, the compound, or the pharmaceutically acceptable salt thereof, is the compound of Formula (I), (Ia), (Ia1), (Ib), (Ib1), (Ic), or (Ic1) wherein each R^{3a} is independently —OH, C₁₋₃ alkoxy, C₁₋₃ haloalkoxy, —NH₂, or —O—C(O)C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein each R^{3a} is independently —OH, C₁₋₃ alkoxy, or C₁₋₃ haloalkoxy. These embodiments of R^{3a} can be combined with any of the embodiments described herein for R³.

In some embodiments, the compound, or the pharmaceutically acceptable salt thereof, is the compound of Formula (I), (Ia), (Ia1), (Ib), (Ib1), (Ic), or (Ic1) wherein at least one R^{3a} is —O—(CH₂CH₂O)₁₋₂-heterocycloalkyl, wherein each heterocycloalkyl has 4 to 6 ring members and 1 to 3 heteroatoms each independently N, O, or S. These embodiments of R^{3a} can be combined with any of the embodiments described herein for R³.

In some embodiments, the compound, or the pharmaceutically acceptable salt thereof, is the compound of Formula (I), (Ia), (Ia1), (Ib), (Ib1), (Ic), or (Ic1) wherein R³ is (b) C₃₋₁₂ cycloalkyl substituted with 0 to 5 R^{3b}. In some embodiments, the compound, or the pharmaceutically acceptable salt thereof, is the compound of Formula (I), (Ia), (Ia1), (Ib), (Ib1), (Ic), or (Ic1) wherein R³ is C₃₋₆ monocyclic cycloalkyl substituted with 0 to 5 R^{3b}. In some embodiments, the compound, or the pharmaceutically acceptable salt thereof, is the compound of Formula (I), (Ia), (Ia1), (Ib), (Ib1), (Ic), or (Ic1) wherein R³ is C₅₋₁₀ fused bicyclic cycloalkyl substituted with 0 to 5 R^{3b}. In some embodiments, the compound, or the pharmaceutically acceptable salt thereof, is the compound of Formula (I), (Ia), (Ia1), (Ib), (Ib1), (Ic), or (Ic1) wherein R³ is C₅₋₁₀ bridged polycyclic cycloalkyl substituted with 0 to 5 R^{3b}. In some embodiments, the compound, or the pharmaceutically acceptable salt thereof, is the compound of Formula (I), (Ia), (Ia1), (Ib), (Ib1), (Ic), or (Ic1) wherein R³ is C₅₋₁₀ spirocycloalkyl substituted with 0 to 5 R^{3b}. These embodiments of R³ can be combined with any of the embodiments described herein for R^{3b}.

In some embodiments, the compound, or the pharmaceutically acceptable salt thereof, is the compound of Formula (I), (Ia), (Ia1), (Ib), (Ib1), (Ic), or (Ic1) wherein R³ is substituted with 0 R^{3b} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 1 R^{3b} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 2 R^{3b} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 3 R^{3b} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 4 R^{3b} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 5 R^{3b} groups. These embodiments of R³ can be combined with any of the embodiments described herein for R^{3b}.

In some embodiments, the compound, or the pharmaceutically acceptable salt thereof, is the compound of Formula (I), (Ia), (Ia1), (Ib), (Ib1), (Ic), or (Ic1) wherein each R^{3b} is independently C₁₋₄ alkyl, C₂₋₄ alkynyl, halo, C₁₋₄ haloalkyl, or cyano. In some embodiments, the compound, or the pharmaceutically acceptable salt thereof, is the compound of Formula (I), (Ia), (Ia1), (Ib), (Ib1), (Ic), or (Ic1) wherein each R^{3b} is independently C₁₋₄ alkyl, halo, or C₁₋₄ haloalkyl. In some embodiments, the compound, or the pharmaceutically acceptable salt thereof, is the compound of Formula (I), (Ia), (Ia1), (Ib), (Ib1), (Ic), or (Ic1) wherein each R^{3b} is C₁₋₄ haloalkyl. These embodiments of R^{3b} can be combined with any of the embodiments described herein for R³.

In some embodiments, the compound, or the pharmaceutically acceptable salt thereof, is the compound of Formula (I), (Ia), (Ia1), (Ib), (Ib1), (Ic), or (Ic1) wherein R³ is (c) heterocycloalkyl having 3 to 6 ring members and 1 to 3 heteroatoms each independently N, O, or S, wherein the heterocycloalkyl is substituted with 0 to 5 R^{3c}. In some embodiments, the compound, or the pharmaceutically acceptable salt thereof, is the compound of Formula (I), (Ia), (Ia1), (Ib), (Ib1), (Ic), or (Ic1) wherein R³ is monocyclic heterocycloalkyl having 3 to 6 ring members and 1 to 3 heteroatoms each independently N, O, or S, wherein the heterocycloalkyl is substituted with 0 to 5 R^{3c}. In some embodiments, the compound, or the pharmaceutically acceptable salt thereof, is the compound of Formula (I), (Ia), (Ia1), (Ib), (Ib1), (Ic), or (Ic1) wherein R³ is monocyclic heterocycloalkyl having 4 to 6 ring members and 1 to 2 heteroatoms each independently O or S, wherein the heterocycloalkyl is substituted with 0 to 5 R^{3c}. These embodiments of R³ can be combined with any of the embodiments described herein for R^{3c}.

In some embodiments, the compound, or the pharmaceutically acceptable salt thereof, is the compound of Formula (I), (Ia), (Ia1), (Ib), (Ib1), (Ic), or (Ic1) wherein R³ is substituted with 0 R^{3c} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 1 R^{3c} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 2 R^{3c} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 3 R^{3c} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 4 R^{3c} groups. In some embodiments, the compound, or the pharmaceutically acceptable salt thereof, is the compound of Formula (I), (Ia), (Ia1), (Ib), (Ib1), (Ic), or (Ic1) wherein R³ is substituted with 5 R^{3c} groups. These embodiments of R³ can be combined with any of the embodiments described herein for R^{3c}.

In some embodiments, the compound, or the pharmaceutically acceptable salt thereof, is the compound of Formula (I), (Ia), (Ia1), (Ib), (Ib1), (Ic), or (Ic1) wherein each R^{3c} is independently C₁₋₄ alkyl, C₁₋₄ haloalkyl, or oxo. In some embodiments, the compound, or the pharmaceutically acceptable salt thereof, is the compound of Formula (I), (Ia), (Ia1), (Ib), (Ib1), (Ic), or (Ic1) wherein each R^{3c} is independently C₁₋₄ alkyl or C₁₋₄ haloalkyl. These embodiments of R^{3c} can be combined with any of the embodiments described herein for R³.

The embodiments described herein for R^{3a} can be present in combination with any embodiment described herein of R³ being (a) C₁₋₈ alkyl, C₂₋₈ alkynyl, or C₁₋₈ haloalkyl, each substituted with 0 to 5 R^{3a}. The embodiments described herein for R^{3b} can be present in combination with any embodiment described herein of R³ being (b) C₃₋₁₂ cycloalkyl substituted with 0 to 5 R^{3b}. The embodiments described herein for R^{3c} can be present in combination with any embodiment described herein of R³ being (c) heterocycloalkyl having 3 to 6 ring members and 1 to 3 heteroatoms each independently N, O, or S, wherein the heterocycloalkyl is substituted with 0 to 5 R^{3c}.

In some embodiments, the compound, or the pharmaceutically acceptable salt thereof, is the compound of Formula (I), (Ia), (Ia1), (Ib), (Ib1), (Ic), or (Ic1) wherein R³ is

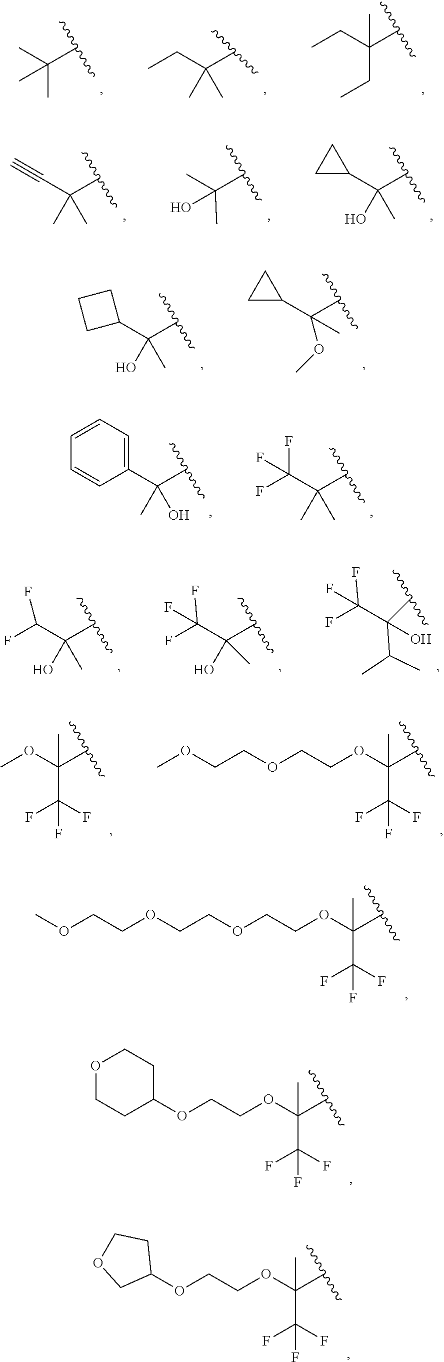

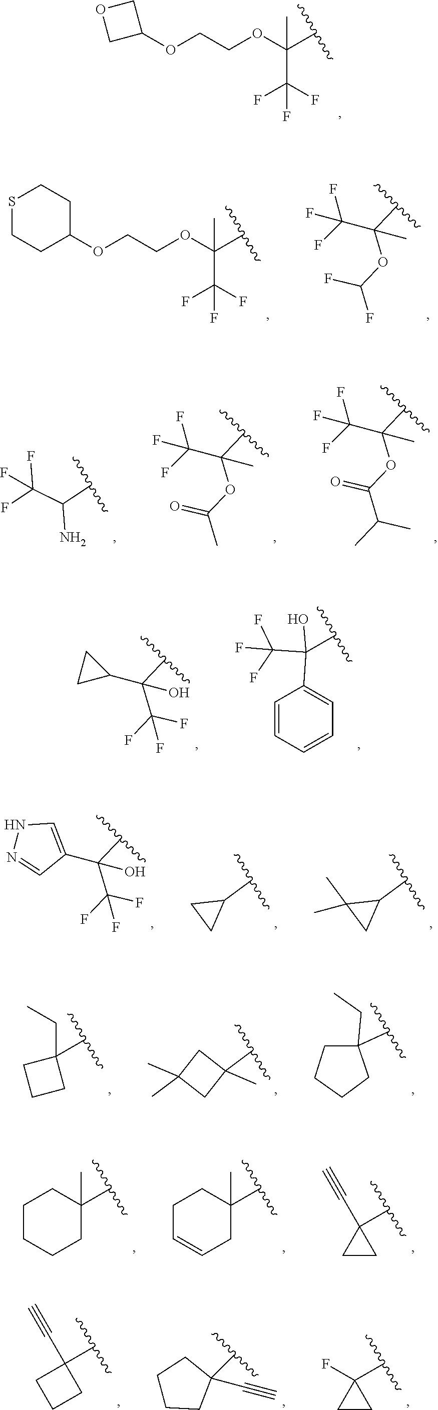

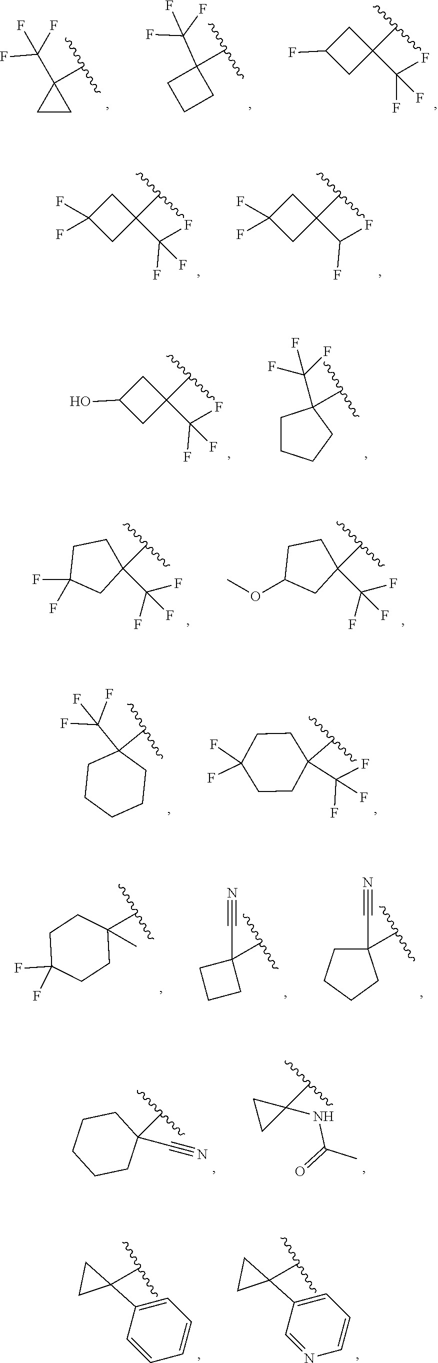

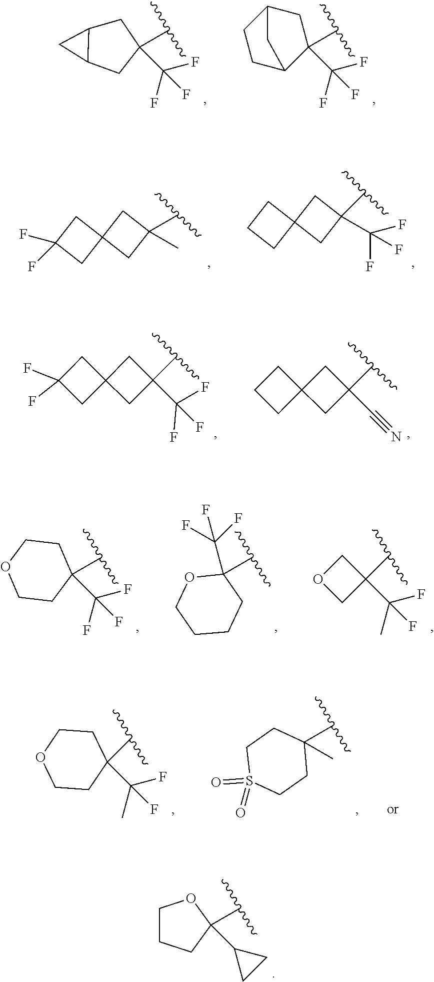

In some embodiments, the compound, or the pharmaceutically acceptable salt thereof, is the compound of Formula (I), (Ia), (Ia1), (Ib), (Ib1), (Ic), or (Ic1) wherein R³ is

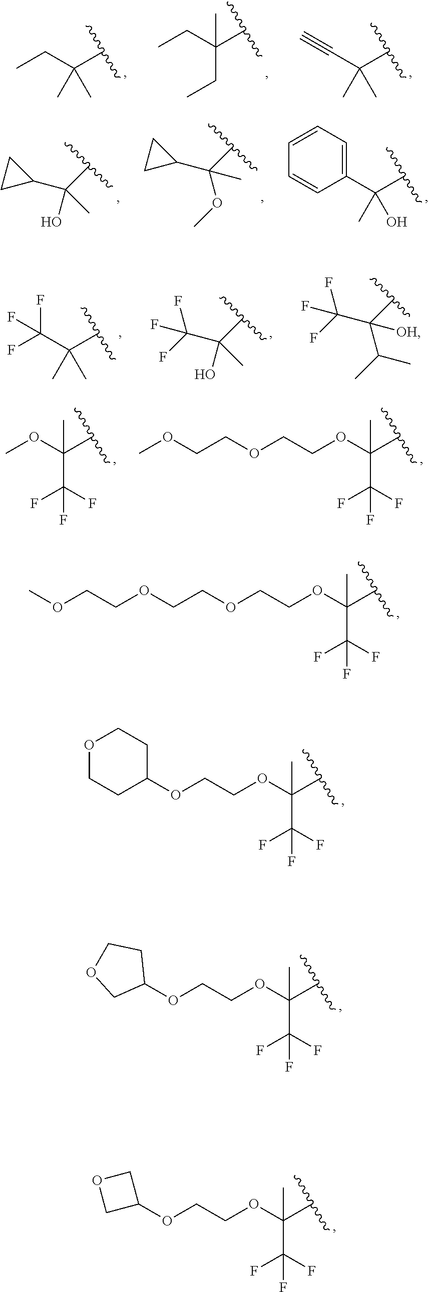

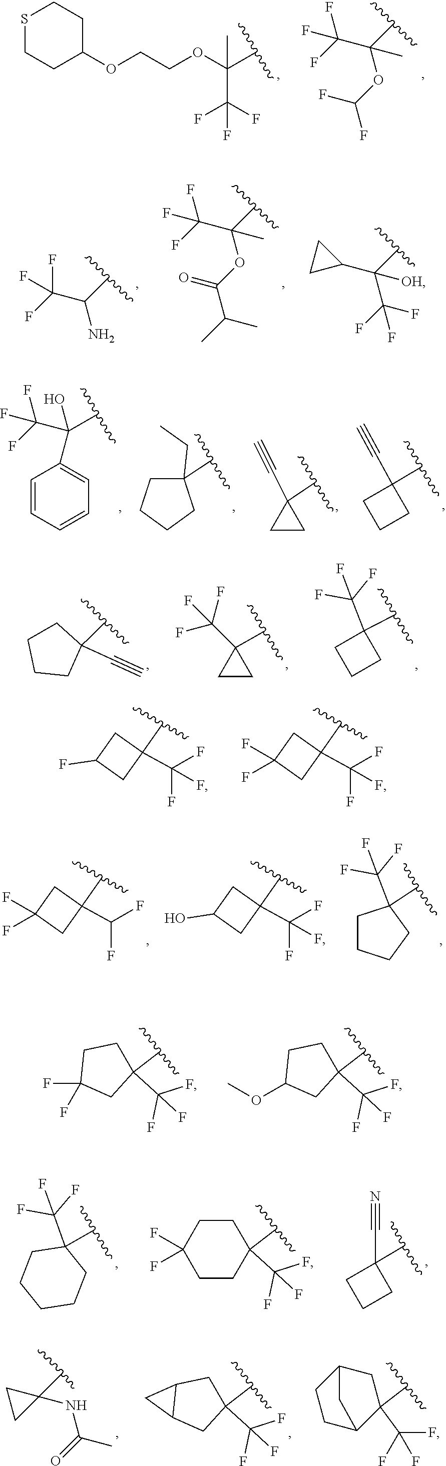

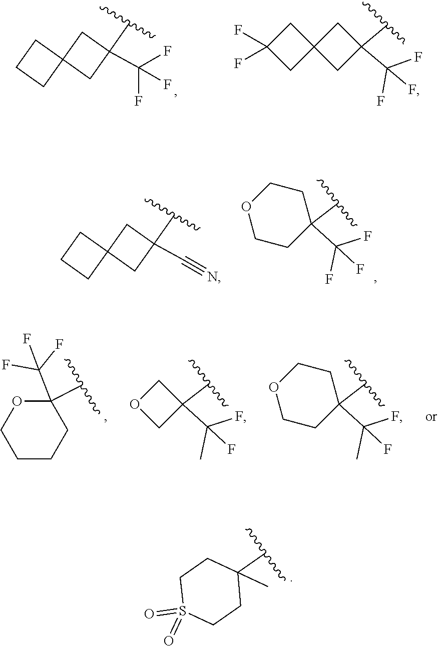

In some embodiments, the compound, or the pharmaceutically acceptable salt thereof, is the compound of Formula (I), (Ia), (Ia1), (Ib), (Ib1), (Ic), or (Ic1) wherein R³ is

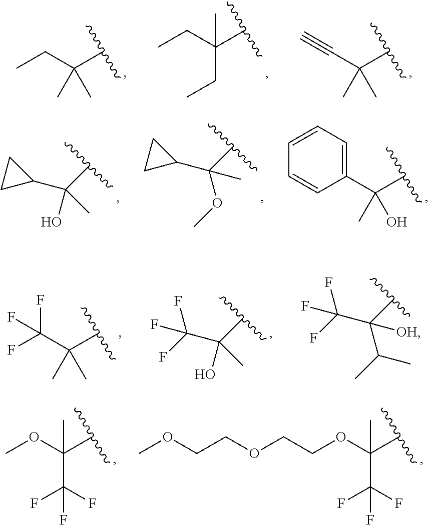

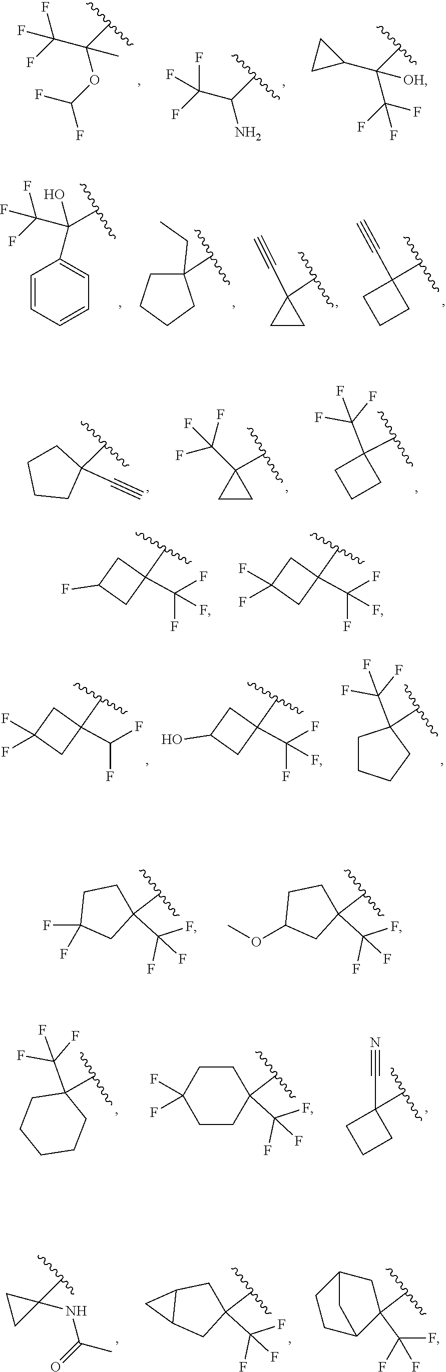

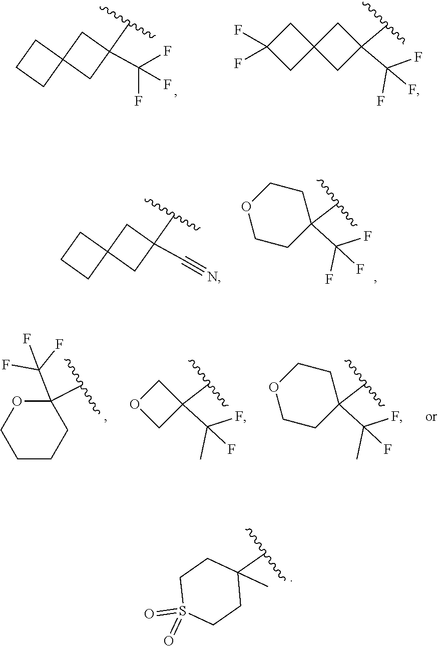

Any of the embodiments described herein for residue 3 can be combined with any of the embodiments described herein for residues 4, 5, 6, 7, 8, and 9. For example, any of the embodiments of R³ as described herein, can be combined with any of the embodiments described herein for R^{4a}, R^{4b}, R^{4c}, R^{5a}, R^{5b}, R^{5c}, X⁶, R^{6a}, R^{6b}, R^{6d}, R^{7a}, R^{7b}, R^{7c}, R^{8a}, R^{8b}, R^{8d}, R^{8e}, ring B, m8, R^{8f}, X⁹, R^{9a}, R^{9b}, and R^{9c}.

### Residue 4

In some embodiments, the compound, or the pharmaceutically acceptable salt thereof, is the compound of Formula (I), (Ia), (Ia1), (Ib), (Ib1), (Ic), or (Ic1) wherein R^{4a} is H or C₁₋₄ alkyl;
R^{4b} and R^{4c} are each independently H, C₁₋₈ alkyl, C₁₋₈ alkyl-OH, C₁₋₄ alkyl-NR^{4c1}R^{4c2}, C₃₋₆ cycloalkyl, C₁₋₄ alkyl-C₃₋₆ cycloalkyl, C₁₋₄ alkyl-heterocycloalkyl, or C₁₋₄ alkyl-heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O, or S;
alternatively R^{4c} and R^{4a} together with the carbon and nitrogen to which each is attached combine to form a heterocycloalkyl having 4 to 6 ring members and 0 to 2 additional heteroatoms each independently N, O or S, wherein the heterocycloalkyl is substituted with 0 to 2 R^{4a1};
each R^{4c1} and R^{4c2} are independently C₁₋₄ alkyl or C₂₋₆ alkoxyalkyl; each R^{4a1} is independently C₁₋₄ alkyl, —OH, C₁₋₄ alkoxy, halo, or —N(H)S(O)₂—C₁₋₄ alkyl;
alternatively, two R^{4a1} groups on adjacent ring atoms combine to form a phenyl ring substituted with 0 to 2 R^{4a3}; and
each R^{4a3} is independently —OH, C₁₋₄ alkyl-OH, or C₁₋₄ alkoxy.

In some embodiments, the compound, or the pharmaceutically acceptable salt thereof, is the compound of Formula (I), (Ia), (Ia1), (Ib), (Ib1), (Ic), or (Ic1) wherein
R^{4a} is H or C₁₋₄ alkyl;
R^{4b} and R^{4c} are each independently H, C₁₋₈ alkyl, C₁₋₈ alkyl-OH, C₃₋₆ cycloalkyl, C₁₋₄ alkyl-C₃₋₆ cycloalkyl, C₁₋₄ alkyl-heterocycloalkyl, or C₁₋₄ alkyl-heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O, or S;
alternatively R^{4c} and R^{4a} together with the carbon and nitrogen to which each is attached combine to form a heterocycloalkyl having 4 to 6 ring members and 0 to 2 additional heteroatoms each independently N, O or S, wherein the heterocycloalkyl is substituted with 0 to 2 R^{4a1};
each R^{4a1} is independently C₁₋₄ alkyl, —OH, C₁₋₄ alkoxy, halo, or —N(H)S(O)₂—C₁₋₄ alkyl;
alternatively, two R^{4a1} groups on adjacent ring atoms combine to form a phenyl ring substituted with 0 to 2 R^{4a3}; and
each R^{4a3} is independently —OH, C₁₋₄ alkyl-OH, or C₁₋₄ alkoxy.

In some embodiments, the compound, or the pharmaceutically acceptable salt thereof, is the compound of Formula (I), (Ia), (Ia1), (Ib), (Ib1), (Ic), or (Ic1) wherein
R^{4a} is H or C₁₋₄ alkyl;
R^{4b} and R^{4c} are each independently H, C₁₋₈ alkyl, or C₁₋₄ alkyl-NR^{4c1}R^{4c2};
alternatively R^{4c} and R^{4a} together with the carbon and nitrogen to which each is attached combine to form a heterocycloalkyl having 4 to 6 ring members and 0 to 2 additional heteroatoms each independently N, O or S, wherein the heterocycloalkyl is substituted with 0 to 2 R^{4a1};
each R^{4c1} and R^{4c2} are independently C₁₋₄ alkyl;
each R^{4a1} is independently —OH, or halo;
alternatively, two R^{4a1} groups on adjacent ring atoms combine to form a phenyl ring substituted with 0 to 2 R^{4a3}; and
each R^{4a3} is —OH.

In some embodiments, the compound, or the pharmaceutically acceptable salt thereof, is the compound of Formula (I), (Ia), (Ia1), (Ib), (Ib1), (Ic), or (Ic1) wherein
R^{4a} is H or C₁₋₄ alkyl;
R^{4b} and R^{4c} are each independently H or C₁₋₈ alkyl;
alternatively R^{4c} and R^{4a} together with the carbon and nitrogen to which each is attached combine to form a heterocycloalkyl having 4 to 6 ring members and 0 to 2 additional heteroatoms each independently N, O or S, wherein the heterocycloalkyl is substituted with 0 to 2 R^{4a1};
each R^{4c1} and R^{4c2} are independently C₁₋₄ alkyl;
each R^{4a1} is independently —OH, or halo;
alternatively, two R^{4a1} groups on adjacent ring atoms combine to form a phenyl ring substituted with 0 to 2 R^{4a3}; and
each R^{4a3} is —OH.

In some embodiments, the compound, or the pharmaceutically acceptable salt thereof, is the compound of Formula (I), (Ia), (Ia1), (Ib), (Ib1), (Ic), or (Ic1) wherein R^{4a} is H. In some embodiments, the compound, or the pharmaceutically acceptable salt thereof, is the compound of Formula (I), (Ia), (Ia1), (Ib), (Ib1), (Ic), or (Ic1) wherein R^{4a} is C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{4a} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{4a} is methyl. In some embodiments, the compound, or the pharmaceutically acceptable salt thereof, is the compound of Formula (I), (Ia), (Ia1), (Ib), (Ib1), (Ic), or (Ic1) wherein R^{4a} is ethyl. These embodiments of R^{4a} can be combined with any of the embodiments described herein for R^{4b} and R^{4c}.

In some embodiments, the compound, or the pharmaceutically acceptable salt thereof, is the compound of Formula (I), (Ia), (Ia1), (Ib), or (Ib1) wherein R^{4b} is H, C₁₋₈ alkyl, or C₁₋₄ alkyl-NR^{4c1}R^{4c2}. In some embodiments, the compound, or the pharmaceutically acceptable salt thereof, is the compound of Formula (I), (Ia), (Ia1), (Ib), or (Ib1) wherein R^{4b} is C₁₋₈ alkyl. In some embodiments, the compound, or the pharmaceutically acceptable salt thereof, is the compound of Formula (I), (Ia), (Ia1), (Ib), or (Ib1) wherein R^{4b} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), or (Ib1) wherein R^{4b} is H. These embodiments of R^{4b} can be combined with any of the embodiments described herein for R^{4a} and R^{4c}.

In some embodiments, the compound, or the pharmaceutically acceptable salt thereof, is the compound of Formula (I), (Ia), (Ia1), (Ib), (Ib1), (Ic), or (Ic1) wherein R^{4c} is C₁₋₈ alkyl, —C₁₋₄ alkyl-NR^{4c1}R^{4c2}, or cycloalkyl. In some embodiments, the compound, or the pharmaceutically acceptable salt thereof, is the compound of Formula (I), (Ia), (Ia1), (Ib), (Ib1), (Ic), or (Ic1) wherein each R^{4c1} and R^{4c2} are independently C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{4c} is C₁₋₈ alkyl or C₃₋₆ cycloalkyl. In some embodiments, the compound, or the pharmaceutically acceptable salt thereof, is the compound of Formula (I), (Ia), (Ia1), (Ib), (Ib1), (Ic), or (Ic1) wherein R^{4c} is C₁₋₈ alkyl. In some embodiments, the compound, or the pharmaceutically acceptable salt thereof, is the compound of Formula (I), (Ia), (Ia1), (Ib), (Ib1), (Ic), or (Ic1) wherein R^{4c} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{4c} is C₃₋₆ monocyclic cycloalkyl. In some embodiments, the compound, or the pharmaceutically acceptable salt thereof, is the compound of Formula (I), (Ia), (Ia1), (Ib), (Ib1), (Ic), or (Ic1) wherein R^{4c} is cyclopropyl, cyclobutyl, cyclopentyl, or cyclohexyl. These embodiments of R^{4c} can be combined with any of the embodiments described herein for R^{4a} and R^{4b}.

In some embodiments, the compound, or the pharmaceutically acceptable salt thereof, is the compound of Formula (I), (Ia), (Ia1), (Ib), (Ib1), (Ic), or (Ic1) wherein R^{4c} and R^{4a} together with the carbon and nitrogen to which each is attached combine to form a heterocycloalkyl having 4 to 6 ring members and 0 to 2 additional heteroatoms each independently N, O or S, wherein the heterocycloalkyl is substituted with 0 to 2 R^{4a1}. In some embodiments, the compound, or the pharmaceutically acceptable salt thereof, is the compound of Formula (I), (Ia), (Ia1), (Ib), (Ib1), (Ic), or (Ic1) wherein R^{4c} and R^{4a} together with the carbon and nitrogen to which each is attached combine to form a heterocycloalkyl selected from pyrrolidinyl, azetidinyl, and piperidinyl, wherein the heterocycloalkyl is substituted with 0 to 2 R^{4a1}. In some embodiments, the compound, or the pharmaceutically acceptable salt thereof, is the compound of Formula (I), (Ia), (Ia1), (Ib), (Ib1), (Ic), or (Ic1) wherein R^{4c} and R^{4a} together with the carbon and nitrogen to which each is attached combine to form pyrrolidinyl, wherein the pyrrolidinyl is substituted with 0 to 2 R^{4a1}. In some embodiments, the compound, or the pharmaceutically acceptable salt thereof, is the compound of Formula (I), (Ia), (Ia1), (Ib), (Ib1), (Ic), or (Ic1) wherein R^{4c} and R^{4a} together with the carbon and nitrogen to which each is attached combine to form azetidinyl, wherein the azetidinyl is substituted with 0 to 2 R^{4a1}. In some embodiments, the compound, or the pharmaceutically acceptable salt thereof, is the compound of Formula (I), (Ia), (Ia1), (Ib), (Ib1), (Ic), or (Ic1) wherein R^{4c} and R^{4a} together with the carbon and nitrogen to which each is attached combine to form piperidinyl, wherein the piperidinyl is substituted with 0 to 2 R^{4a1}. These embodiments of R^{4a} and R^{4c} can be combined with any of the embodiments described herein for R^{4b}.

In some embodiments, the compound, or the pharmaceutically acceptable salt thereof, is the compound of Formula (I), (Ia), (Ia1), (Ib), (Ib1), (Ic), or (Ic1) wherein the heterocycloalkyl comprising R^{4a}/R^{4c} is substituted with 0 R^{4a1}. In some embodiments, the compound, or the pharmaceutically acceptable salt thereof, is the compound of Formula (I), (Ia), (Ia1), (Ib), (Ib1), (Ic), or (Ic1) wherein the heterocycloalkyl comprising R^{4a}/R^{4c} is substituted with 1 R^{4a1}. In some embodiments, the compound, or the pharmaceutically acceptable salt thereof, is the compound of Formula (I), (Ia), (Ia1), (Ib), (Ib1), (Ic), or (Ic1) wherein the heterocycloalkyl comprising R^{4a}/R^{4c} is substituted with 2 R^{4a1}. These embodiments of R^{4a} and R^{4c} can be combined with any of the embodiments described herein for R^{4b}.

In some embodiments, the compound, or the pharmaceutically acceptable salt thereof, is the compound of Formula (I), (Ia), (Ia1), (Ib), (Ib1), (Ic), or (Ic1) wherein each R^{4a1} is independently C₁₋₄ alkyl, —OH, C₁₋₄ alkoxy, halo, or —N(H)S(O)₂—C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein each R^{4a1} is independently C₁₋₄ alkyl, —OH, C₁₋₄ alkoxy, or halo. In some embodiments, the compound, or the pharmaceutically acceptable salt thereof, is the compound of Formula (I), (Ia), (Ia1), (Ib), (Ib1), (Ic), or (Ic1) wherein each R^{4a1} is independently C₁₋₄ alkyl or halo. In some embodiments, the compound, or the pharmaceutically acceptable salt thereof, is the compound of Formula (I), (Ia), (Ia1), (Ib), (Ib1), (Ic), or (Ic1) wherein each R^{4a1} is independently —OH or halo. These embodiments of R^{4a1} can be combined with any of the embodiments described herein for R^{4b} and combined R^{4a} and R^{4c}.

In some embodiments, the compound, or the pharmaceutically acceptable salt thereof, is the compound of Formula (I), (Ia), (Ia1), (Ib), (Ib1), (Ic), or (Ic1) wherein two R^{4a1} groups on adjacent ring atoms combine to form a phenyl ring substituted with 0 to 2 R^{4a3}. In some embodiments, the compound, or the pharmaceutically acceptable salt thereof, is the compound of Formula (I), (Ia), (Ia1), (Ib), (Ib1), (Ic), or (Ic1) wherein the phenyl ring is substituted with 0 R^{4a3}. In some embodiments, the compound, or the pharmaceutically acceptable salt thereof, is the compound of Formula (I), (Ia), (Ia1), (Ib), (Ib1), (Ic), or (Ic1) wherein the phenyl ring is substituted with 1 R^{4a3}. In some embodiments, the compound, or the pharmaceutically acceptable salt thereof, is the compound of Formula (I), (Ia), (Ia1), (Ib), (Ib1), (Ic), or (Ic1) wherein the phenyl ring is substituted with 2 R^{4a3}. These embodiments of R^{4a1} can be combined with any of the embodiments described herein for R^{4b} and combined R^{4a} and R^{4c}.

In some embodiments, the compound, or the pharmaceutically acceptable salt thereof, is the compound of Formula (I), (Ia), (Ia1), (Ib), (Ib1), (Ic), or (Ic1) wherein each R^{4a3} is independently —OH, C₁₋₄ alkyl-OH, or C₁₋₄ alkoxy. In some embodiments, the compound, or the pharmaceutically acceptable salt thereof, is the compound of Formula (I), (Ia), (Ia1), (Ib), (Ib1), (Ic), or (Ic1) wherein each R^{4a3} is independently —OH. In some embodiments, the compound, or the pharmaceutically acceptable salt thereof, is the compound of Formula (I), (Ia), (Ia1), (Ib), (Ib1), (Ic), or (Ic1) wherein each R^{4a3} is independently C₁₋₄ alkyl-OH. In some embodiments, the compound, or the pharmaceutically acceptable salt thereof, is the compound of Formula (I), (Ia), (Ia1), (Ib), (Ib1), (Ic), or (Ic1) wherein each R^{4a3} is independently C₁₋₄ alkoxy. These embodiments of R^{4a3} can be combined with any of the embodiments described herein for two combined R^{4a1} groups, combined R^{4c} and R^{4a}, and R^{4b}.

In some embodiments, the compound, or the pharmaceutically acceptable salt thereof, is the compound of Formula (I), (Ia), (Ia1), (Ib), (Ib1), (Ic), or (Ic1) wherein R^{4c} and R^{4a} together with the carbon and nitrogen to which each is attached combine to form pyrrolidinyl substituted 2 R^{4a1} groups, wherein the 2 R^{4a1} groups are on adjacent ring atoms and combine to form a phenyl ring substituted with 0 to 2 R^{4a3}. In some embodiments, the compound, or the pharmaceutically acceptable salt thereof, is the compound of Formula (I), (Ia), (Ia1), (Ib), (Ib1), (Ic), or (Ic1) wherein R^{4c} and R^{4a} together with the carbon and nitrogen to which each is attached combine to form azetidinyl substituted 2 R^{4a1} groups, wherein the 2 R^{4a1} groups are on adjacent ring atoms and combine to form a phenyl ring substituted with 0 to 2 R^{4a3}. In some embodiments, the compound, or the pharmaceutically acceptable salt thereof, is the compound of Formula (I), (Ia), (Ia1), (Ib), (Ib1), (Ic), or (Ic1) wherein R^{4c} and R^{4a} together with the carbon and nitrogen to which each is attached combine to form piperidinyl substituted 2 R^{4a1} groups, wherein the 2 R^{4a1} groups are on adjacent ring atoms and combine to form a phenyl ring substituted with 0 to 2 R^{4a3}. These embodiments of combined R^{4c} and R^{4a}, and two combined R^{4a1} groups can be combined with any of the embodiments described herein for R^{4b} and R^{4a3}.

In some embodiments, the compound, or the pharmaceutically acceptable salt thereof, is the compound of Formula (I), (Ia), (Ia1), (Ib), (Ib1), (Ic), or (Ic1) wherein
R^{4a} is H or methyl;
R^{4b} is H;
R^{4c} is methyl, ethyl, isopropyl, tert-butyl,

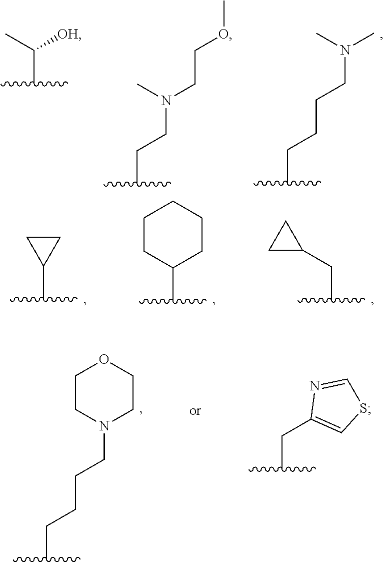

alternatively R^{4c} and R^{4a} together with the carbon and nitrogen to which each is attached combine to form a heterocycloalkyl having 4 to 6 ring members and 0 to 1 additional oxygen, wherein the heterocycloalkyl is substituted with 0 to 2 R^{4a1}; and
each R^{4a1} is independently methyl, —OH, methoxy, fluoro, or —N(H)S(O)₂CH₃;
alternatively, two R^{4a1} groups on adjacent ring atoms combine to form a phenyl ring substituted with 0 to 2 —OH.

In some embodiments, the compound, or the pharmaceutically acceptable salt thereof, is the compound of Formula (I), (Ia), (Ia1), (Ib), (Ib1), (Ic), or (Ic1) wherein R^{4a}, R^{4b}, and R^{4c} are as follows:
R^{4a} is H or methyl;
R^{4b} is H;
R^{4c} is methyl, ethyl, isopropyl,

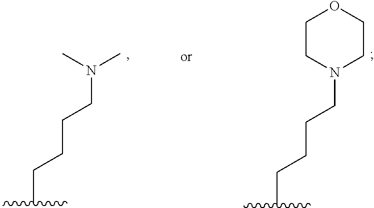

alternatively R^{4c} and R^{4a} together with the carbon and nitrogen to which each is attached combine to form a heterocycloalkyl having 4 to 6 ring members and 0 additional heteroatoms, wherein the heterocycloalkyl is substituted with 0 to 2 R^{4a1}; and
each R^{4a1} is independently-OH or fluoro;
alternatively, two R^{4a1} groups on adjacent ring atoms combine to form a phenyl ring substituted with 0 to 1 —OH.

The embodiments described herein for R^{4a}, R^{4b} and R^{4c} can be present in any combination. In addition, the embodiments described herein for residue 4 can be present in combination with any of the embodiments described herein for residues 3, 5, 6, 7, 8, and 9. For example, any of the embodiments of R^{4a}, R^{4b} and R^{4c} as described herein, can be combined with any of the embodiments described herein for R³, R^{5a}, R^{5b}, R^{5c}, X⁶, R^{6a}, R^{6b}, R^{6d}, R^{7a}, R^{7b}, R^{7c}, R^{8a}, R^{8b}, R^{8d}, R^{8e}, ring B, m8, R^{8f}, X⁹, R^{9a}, R^{9b}, and R^{9c}.

### Residue 5

In some embodiments, the compound, or the pharmaceutically acceptable salt thereof, is the compound of Formula (I), (Ia), (Ia1), (Ib), (Ib1), (Ic), or (Ic1) wherein
R^{5a} is H;
R^{5b} and R^{5c} are each independently H, C₁₋₈ alkyl, C₁₋₈ alkyl-OH, C₂₋₆ alkoxyalkyl, C₁₋₈ haloalkyl, —C₁₋₄ alkyl-NR^{5b1}R^{5b2}, —C₁₋₃ alkyl-C(O)NR^{5b1}R^{5b2}, —C₁₋₄ alkyl-N(R^{5b3})C(O)R^{5b4}, C₃₋₆ cycloalkyl, or C₁₋₄ alkyl-C₃₋₆ cycloalkyl, wherein each cycloalkyl is substituted with 0 to 3 R^{5b5};
each R^{5b1} and R^{5b2} are independently H, C₁₋₄ alkyl, C₁₋₄ haloalkyl, —C(O)C₁₋₄ alkyl, or —C(O)C₁₋₄ haloalkyl, provided that no more than one of R^{5b1} and R^{5b2} is H;
alternatively, R^{5b1} and R^{5b2} on the same nitrogen atom combine to form a heterocycloalkyl having 6 ring members and 0 to 1 additional oxygen ring members, wherein the heterocycloalkyl is substituted with 0 to 2 R^{5b5};
each R^{5b3} is H or C₁₋₄ alkyl;
each R^{5b4} is a heteroaryl having 5 to 6 ring members and 1 to 3 heteroatoms each independently N, O or S, substituted with 0 to 1 R^{5b5}; and
each R^{5b5} is independently C₁₋₄ alkyl, halo, C₁₋₄ haloalkyl, or NH(CH₃).

In some embodiments, the compound, or the pharmaceutically acceptable salt thereof, is the compound of Formula (I), (Ia), (Ia1), (Ib), (Ib1), (Ic), or (Ic1) wherein
R^{5b} and R^{5c} are each independently H, C₁₋₈ alkyl, C₁₋₈ alkyl-OH, C₂₋₆ alkoxyalkyl, C₁₋₈ haloalkyl, C₃₋₆ cycloalkyl, or C₁₋₄ alkyl-C₃₋₆ cycloalkyl, wherein each cycloalkyl is substituted with 0 to 3 R^{5b5};
each R^{5b5} is independently C₁₋₄ alkyl, halo, or C₁₋₄ haloalkyl.

In some embodiments, the compound, or the pharmaceutically acceptable salt thereof, is the compound of Formula (I), (Ia), (Ia1), (Ib), (Ib1), (Ic), or (Ic1) wherein
R^{5b} and R^{5c} are each independently H, C₁₋₄ alkyl-NR^{5b1}R^{5b2}, C₁₋₃ alkyl-C(O)NR^{5b1}R^{5b2}, or —C₁₋₄ alkyl-N(R^{5b3})C(O)R^{5b4};
each R^{5b1} and R^{5b2} are independently H, C₁₋₄ alkyl, C₁₋₄ haloalkyl, —C(O)C₁₋₄ alkyl, —C(O)C₁₋₄ haloalkyl, provided that no more than one of R^{5b1} and R^{5b2} is H;
alternatively, R^{5b1} and R^{5b2} on the same nitrogen atom combine to form a heterocycloalkyl having 6 ring members and 0 to 1 additional oxygen ring members, wherein the heterocycloalkyl is substituted with 0 to 2 R^{5b5};
each R^{5b3} is H or C₁₋₄ alkyl;
each R^{5b4} is a heteroaryl having 5 to 6 ring members and 1 to 3 heteroatoms each independently N, O or S substituted with 0 to 1 R^{5b5}; and
each R^{5b5} is independently C₁₋₄ alkyl, halo, C₁₋₄ haloalkyl, or NH(CH₃).

In some embodiments, the compound, or the pharmaceutically acceptable salt thereof, is the compound of Formula (I), (Ia), (Ia1), (Ib), (Ib1), (Ic), or (Ic1) wherein R^{5a} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{5a} is H. These embodiments of R^{5a} can be combined with any of the embodiments described herein for R^{5b} and R^{5c}.

In some embodiments, the compound, or the pharmaceutically acceptable salt thereof, is the compound of Formula (I), (Ia), (Ia1), (Ib), (Ib1), (Ic), or (Ic1) wherein R^{5b} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{5b} is H. These embodiments of R^{5b} can be combined with any of the embodiments described herein for R^{5a} and R^{5c}.

In some embodiments, the compound, or the pharmaceutically acceptable salt thereof, is the compound of Formula (I), (Ia), (Ia1), (Ib), (Ib1), (Ic), or (Ic1) wherein R^{5c} is C₁₋₈ alkyl, C₁₋₈ alkyl-OH, C₂₋₆ alkoxyalkyl, C₁₋₈ haloalkyl, C₃₋₆ cycloalkyl, C₁₋₄ alkyl-C₃₋₆ cycloalkyl, wherein each cycloalkyl is substituted with 0 to 3 R^{5b5}. In some embodiments, the compound, or the pharmaceutically acceptable salt thereof, is the compound of Formula (I), (Ia), (Ia1), (Ib), (Ib1), (Ic), or (Ic1) wherein R^{5c} is C₁₋₈ alkyl, C₁₋₈ alkyl-OH, C₂₋₆ alkoxyalkyl, or C₁₋₈ haloalkyl. In some embodiments, the compound, or the pharmaceutically acceptable salt thereof, is the compound of Formula (I), (Ia), (Ia1), (Ib), (Ib1), (Ic), or (Ic1) wherein R^{5c} is C₁₋₈ alkyl, C₁₋₈ alkyl-OH, or C₁₋₈ haloalkyl. In some embodiments, the compound, or the pharmaceutically acceptable salt thereof, is the compound of Formula (I), (Ia), (Ia1), (Ib), (Ib1), (Ic), or (Ic1) wherein R^{5c} is C₃₋₆ cycloalkyl or C₁₋₄ alkyl-C₃₋₆ cycloalkyl, wherein each cycloalkyl is substituted with 0 to 2 halo. In some embodiments, the compound, or the pharmaceutically acceptable salt thereof, is the compound of Formula (I), (Ia), (Ia1), (Ib), (Ib1), (Ic), or (Ic1) wherein R^{5c} is C₃₋₄ cycloalkyl or C₁₋₄ alkyl-C₃₋₄ cycloalkyl, wherein each cycloalkyl is substituted with 0 to 2 halo. In some embodiments, the compound, or the pharmaceutically acceptable salt thereof, is the compound of Formula (I), (Ia), (Ia1), (Ib), (Ib1), (Ic), or (Ic1) wherein R^{5c} is cyclopropyl, cyclobutyl, cyclopropylmethyl, or cyclobutylmethyl substituted with 0 to 2 halo. These embodiments of R^{5c} can be combined with any of the embodiments described herein for R^{5a} and R^{5b}.

In some embodiments, the compound, or the pharmaceutically acceptable salt thereof, is the compound of Formula (I), (Ia), (Ia1), (Ib), (Ib1), (Ic), or (Ic1) wherein R^{5c} is C₁₋₄ alkyl-NR^{5b1}R^{5b2}. In some embodiments, the compound, or the pharmaceutically acceptable salt thereof, is the compound of Formula (I), (Ia), (Ia1), (Ib), (Ib1), (Ic), or (Ic1) wherein R^{5b1} and R^{5b2} are each independently H, C₁₋₄ alkyl, C₁₋₄ haloalkyl, —C(O)C₁₋₄ alkyl, —C(O)C₁₋₄ haloalkyl. In some embodiments, the compound, or the pharmaceutically acceptable salt thereof, is the compound of Formula (I), (Ia), (Ia1), (Ib), (Ib1), (Ic), or (Ic1) wherein at least one of R^{5b1} and R^{5b2} is other than H. In some embodiments, the compound, or the pharmaceutically acceptable salt thereof, is the compound of Formula (I), (Ia), (Ia1), (Ib), (Ib1), (Ic), or (Ic1) wherein each R^{5b1} and R^{5b2} is H. In some embodiments, the compound, or the pharmaceutically acceptable salt thereof, is the compound of Formula (I), (Ia), (Ia1), (Ib), (Ib1), (Ic), or (Ic1) wherein R^{5b1} and R^{5b2} on the same nitrogen atom combine to form a heterocycloalkyl having 6 ring members and 0 to 1 additional oxygen ring members, wherein the heterocycloalkyl is substituted with 0 to 2 R^{5b5}. These embodiments of R^{5c} can be combined with any of the embodiments described herein for R^{5a} and R^{5b}.

In some embodiments, the compound, or the pharmaceutically acceptable salt thereof, is the compound of Formula (I), (Ia), (Ia1), (Ib), (Ib1), (Ic), or (Ic1) wherein R^{5c} is —C₁₋₃ alkyl-C(O)NR^{5b1}R^{5b2}. In some embodiments, the compound, or the pharmaceutically acceptable salt thereof, is the compound of Formula (I), (Ia), (Ia1), (Ib), (Ib1), (Ic), or (Ic1) wherein R^{5b1} and R^{5b2} are each independently H, C₁₋₄ alkyl, C₁₋₄ haloalkyl, —C(O)C₁₋₄ alkyl, —C(O)C₁₋₄ haloalkyl. In some embodiments, the compound, or the pharmaceutically acceptable salt thereof, is the compound of Formula (I), (Ia), (Ia1), (Ib), (Ib1), (Ic), or (Ic1) wherein at least one of R^{5b1} and R^{5b2} is other than H. In some embodiments, the compound, or the pharmaceutically acceptable salt thereof, is the compound of Formula (I), (Ia), (Ia1), (Ib), (Ib1), (Ic), or (Ic1) wherein each R^{5b1} and R^{5b2} is H. In some embodiments, the compound, or the pharmaceutically acceptable salt thereof, is the compound of Formula (I), (Ia), (Ia1), (Ib), (Ib1), (Ic), or (Ic1) wherein R^{5b1} and R^{5b2} on the same nitrogen atom combine to form a heterocycloalkyl having 6 ring members and 0 to 1 additional oxygen ring members, wherein the heterocycloalkyl is substituted with 0 to 2 R^{5b5}. In some embodiments, the compound, or the pharmaceutically acceptable salt thereof, is the compound of Formula (I), (Ia), (Ia1), (Ib), (Ib1), (Ic), or (Ic1) wherein R^{5b1} and R^{5b2} on the same nitrogen atom combine to form piperidine or morpholine, each substituted with 0 to 2 R^{5b5}. In some embodiments, the compound, or the pharmaceutically acceptable salt thereof, is the compound of Formula (I), (Ia), (Ia1), (Ib), (Ib1), (Ic), or (Ic1) wherein each R^{5b5} is halo. In some embodiments, the compound, or the pharmaceutically acceptable salt thereof, is the compound of Formula (I), (Ia), (Ia1), (Ib), (Ib1), (Ic), or (Ic1) wherein each R^{5b5} is fluoro. These embodiments of R^{5c} can be combined with any of the embodiments described herein for R^{5a} and R^{5b}.

In some embodiments, the compound, or the pharmaceutically acceptable salt thereof, is the compound of Formula (I), (Ia), (Ia1), (Ib), (Ib1), (Ic), or (Ic1) wherein R^{5c} is —C₁₋₄ alkyl-N(R^{5b3})C(O)R^{5b4}. In some embodiments, the compound, or the pharmaceutically acceptable salt thereof, is the compound of Formula (I), (Ia), (Ia1), (Ib), (Ib1), (Ic), or (Ic1) wherein R^{5b3} is H or C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{5b3} is H. In some embodiments, the compound, or the pharmaceutically acceptable salt thereof, is the compound of Formula (I), (Ia), (Ia1), (Ib), (Ib1), (Ic), or (Ic1) wherein R^{5b3} is C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{5b4} is a heteroaryl having 5 to 6 ring members and 1 to 3 heteroatoms each independently N, O or S substituted with 0 to 1 R^{5b5}. In some embodiments, the compound, or the pharmaceutically acceptable salt thereof, is the compound of Formula (I), (Ia), (Ia1), (Ib), (Ib1), (Ic), or (Ic1) wherein R^{5b4} is pyridine, pyrrole, pyrazole, imidazole, thiazole, isothiazole, oxazole, or isoxazole, each substituted with 0 to 1 R^{5b5}. In some embodiments, the compound, or the pharmaceutically acceptable salt thereof, is the compound of Formula (I), (Ia), (Ia1), (Ib), (Ib1), (Ic), or (Ic1) wherein R^{5b5} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{5b5} is methyl. These embodiments of R^{5c} can be combined with any of the embodiments described herein for R^{5a} and R^{5b}.

In some embodiments, the compound, or the pharmaceutically acceptable salt thereof, is the compound of Formula (I), (Ia), (Ia1), (Ib), (Ib1), (Ic), or (Ic1) wherein R^{5c} is H, methyl, ethyl, n-propyl, isopropyl, n-butyl, isobutyl, tert-butyl,

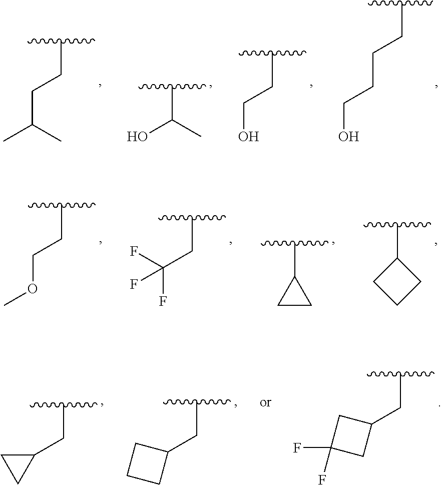

These embodiments of R^{5c} can be combined with any of the embodiments described herein for R^{5a} and R^{5b}.

In some embodiments, R^{5c} is H, methyl, ethyl,

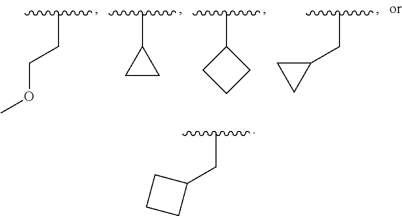

These embodiments of R^{5c} can be combined with any of the embodiments described herein for R^{5a} and R^{5b}.

In some embodiments, the compound, or the pharmaceutically acceptable salt thereof, is the compound of Formula (I), (Ia), (Ia1), (Ib), (Ib1), (Ic), or (Ic1) wherein R^{5c} is

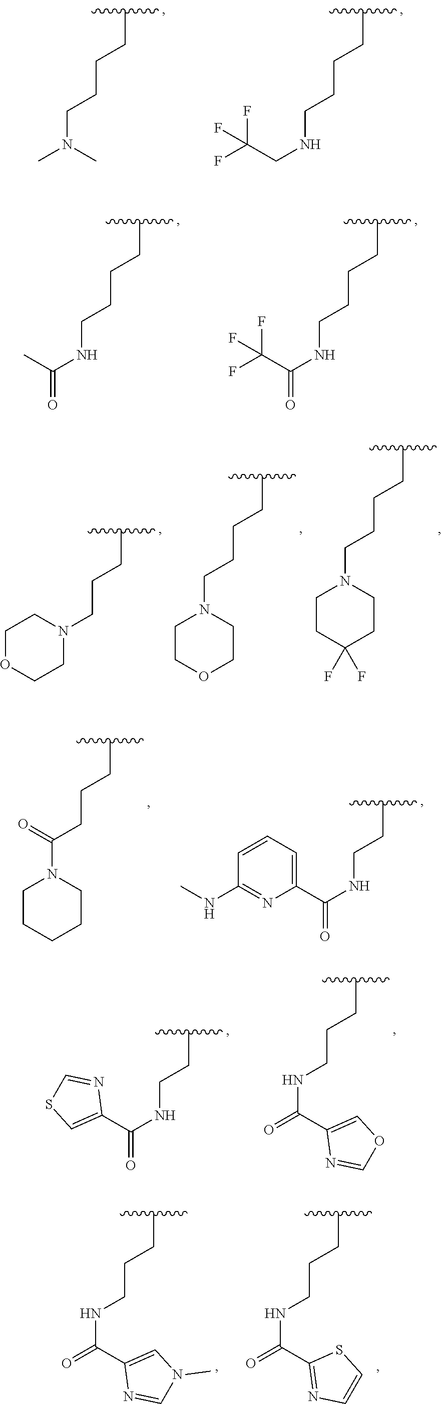

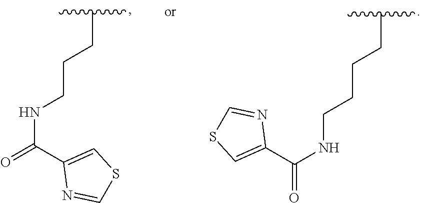 [SMILES: *CCCNC(=O)c1cscn1 ; *CCCCNC(=O)c1cscn1]

These embodiments of R^{5c} can be combined with any of the embodiments described herein for R^{5a} and R^{5b}.

In some embodiments, the compound, or the pharmaceutically acceptable salt thereof, is the compound of Formula (I), (Ia), (Ia1), (Ib), (Ib1), (Ic), or (Ic1) wherein
R^{5a} is H;
R^{5b} is H; and
R^{5c} is H, methyl, ethyl, n-propyl, isopropyl, n-butyl, sec-butyl, isobutyl, tert-butyl,

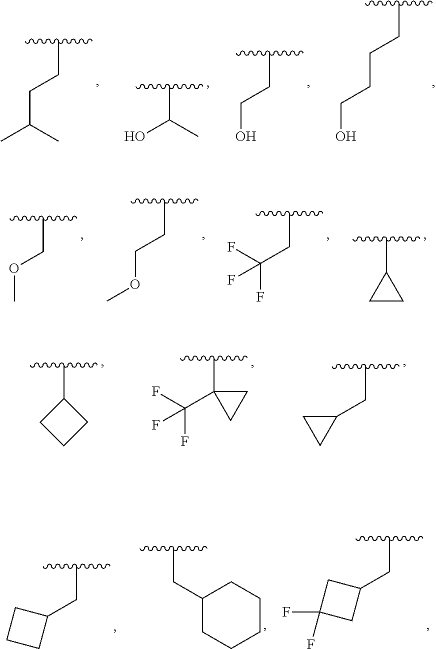

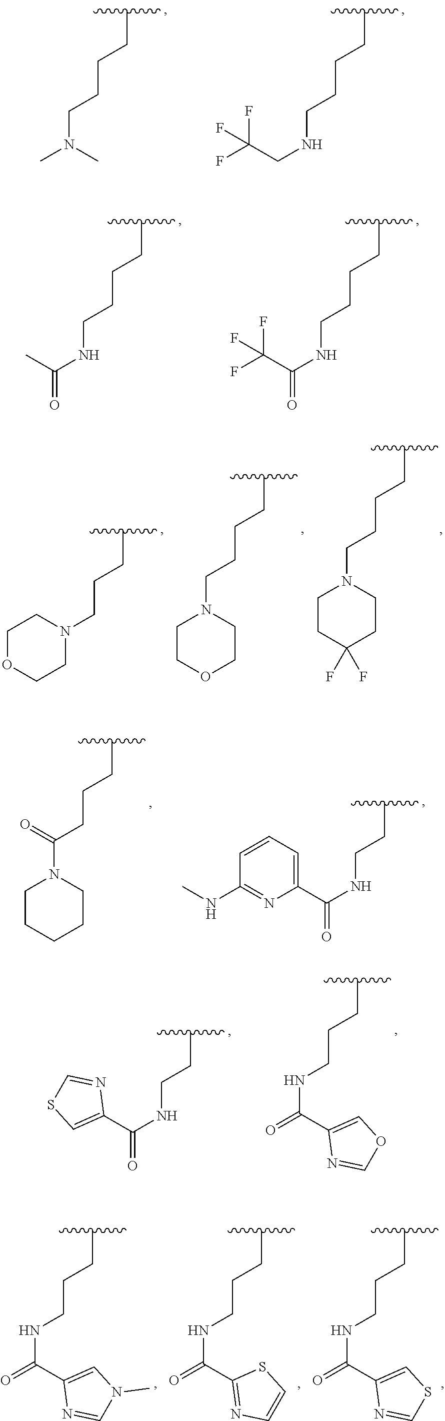

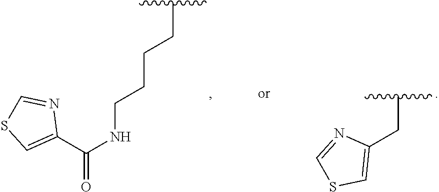 [SMILES: *CCCCNC(=O)c1cscn1 ; *Cc1cscn1]

In some embodiments, the compound, or the pharmaceutically acceptable salt thereof, is the compound of Formula (I), (Ia), (Ia1), (Ib), (Ib1), (Ic), or (Ic1) wherein
R^{5a} is H;
R^{5b} is H; and
R^{5c} is H, methyl, ethyl, n-propyl, isopropyl, n-butyl, isobutyl, tert-butyl,

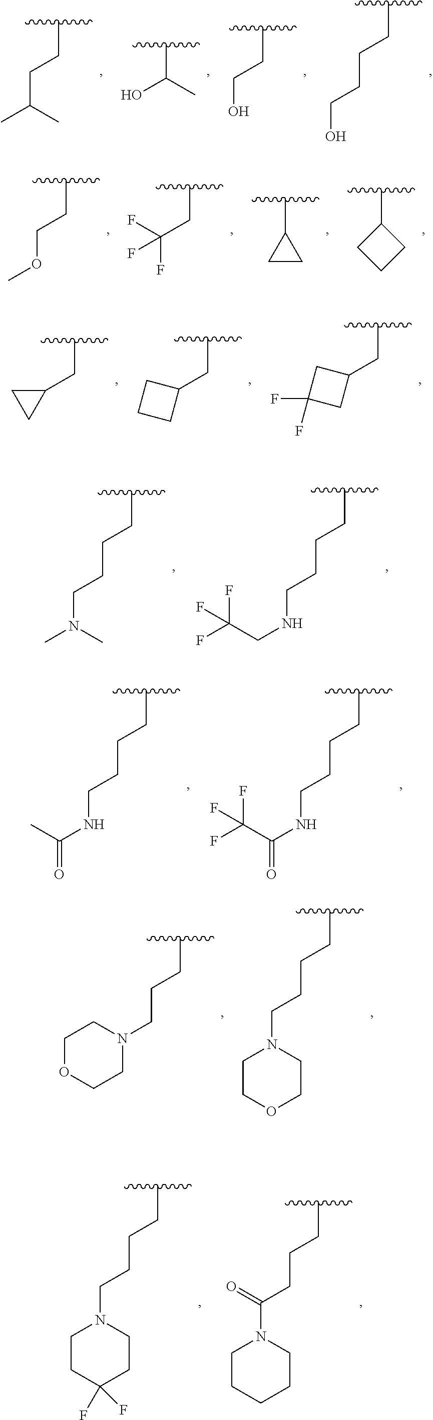

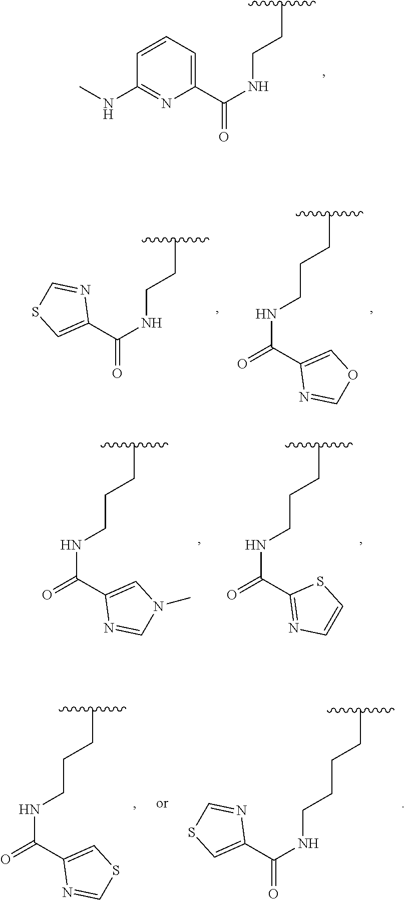

The embodiments described herein for R^{5a}, R^{5b} and R^{5c} can be present in any combination. In addition, the embodiments described herein for residue 5 can be present in combination with any of the embodiments described herein for residues 3, 4, 6, 7, 8, and 9. For example, any of the embodiments of R^{5a}, R^{5b} and R^{5c} as described herein, can be combined with any of the embodiments described herein for R³, R^{4a}, R^{4b}, R^{4c}, X⁶, R^{6a}, R^{6b}, R^{6d}, R^{7a}, R^{7b}, R^{7c}, R^{8a}, R^{8b}, R^{8d}, R^{8e}, ring B, m8, R^{8f}, X⁹, R^{9a}, R^{9b}, and R^{9c}.

### Residue 6

In some embodiments, the compound, or the pharmaceutically acceptable salt thereof, is the compound of Formula (I), (Ia), (Ia1), (Ib), (Ib1), (Ic), or (Ic1) wherein
R^{6a} is H, C₁₋₄ alkyl, C₁₋₄ deuteroalkyl, C₁₋₄ alkyl-C₃₋₆ cycloalkyl, or C₁₋₄ alkyl-heterocycloalkyl, wherein the heterocycloalkyl has 4 to 6 ring members and 1 to 3 heteroatoms each independently N, O, or S;
R^{6b} is H; and
R^{6d} is H, C₁₋₄ alkyl, C₁₋₄ deuteroalkyl, —OH, or C₂₋₆ alkoxyalkyl. These embodiments of R^{6a}, R^{6b} and R^{6d} can be combined with any of the embodiments described herein for X⁶.

In some embodiments, the compound, or the pharmaceutically acceptable salt thereof, is the compound of Formula (I), (Ia), (Ia1), (Ib), (Ib1), (Ic), or (Ic1) wherein
R^{6a} is H, C₁₋₄ alkyl, C₁₋₄ deuteroalkyl, C₁₋₄ alkyl-C₃₋₆ cycloalkyl;
R^{6b} is H; and
R^{6d} is H, C₁₋₄ alkyl, or C₁₋₄ deuteroalkyl. These embodiments of R^{6a}, R^{6b} and R^{6d} can be combined with any of the embodiments described herein for X⁶.

In some embodiments, the compound, or the pharmaceutically acceptable salt thereof, is the compound of Formula (I), (Ia), (Ia1), (Ib), (Ib1), (Ic), or (Ic1) wherein R^{6a} is H, C₁₋₄ alkyl, C₁₋₄ deuteroalkyl, C₁₋₄ alkyl-C₃₋₆ cycloalkyl. In some embodiments, the compound, or the pharmaceutically acceptable salt thereof, is the compound of Formula (I), (Ia), (Ia1), (Ib), (Ib1), (Ic), or (Ic1) wherein R^{6a} is C₁₋₄ alkyl, C₁₋₄ deuteroalkyl, C₁₋₄ alkyl-C₃₋₆ cycloalkyl. In some embodiments, the compound, or the pharmaceutically acceptable salt thereof, is the compound of Formula (I), (Ia), (Ia1), (Ib), (Ib1), (Ic), or (Ic1) wherein R^{6a} is H or C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{6a} is H. In some embodiments, the compound, or the pharmaceutically acceptable salt thereof, is the compound of Formula (I), (Ia), (Ia1), (Ib), (Ib1), (Ic), or (Ic1) wherein R^{6a} is C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{6a} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{6a} is methyl. These embodiments of R^{6a} can be combined with any of the embodiments described herein for R^{6b}, R^{6d}, and X⁶.

In some embodiments, the compound, or the pharmaceutically acceptable salt thereof, is the compound of Formula (I), (Ia), (Ia1), (Ib), (Ib1), (Ic), or (Ic1) wherein R^{6b} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{6b} is H. These embodiments of R^{6b} can be combined with any of the embodiments described herein for R^{6a}, R^{6d}, and X⁶.

In some embodiments, the compound, or the pharmaceutically acceptable salt thereof, is the compound of Formula (I), (Ia), (Ia1), (Ib), (Ib1), (Ic), or (Ic1) wherein R^{6d} is H, C₁₋₄ alkyl, or C₁₋₄ deuteroalkyl. In some embodiments, the compound, or the pharmaceutically acceptable salt thereof, is the compound of Formula (I), (Ia), (Ia1), (Ib), (Ib1), (Ic), or (Ic1) wherein R^{6d} is H. In some embodiments, the compound, or the pharmaceutically acceptable salt thereof, is the compound of Formula (I), (Ia), (Ia1), (Ib), (Ib1), (Ic), or (Ic1) wherein R^{6d} is C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{6d} is methyl, ethyl, n-propyl, iso-propyl, n-butyl, iso-butyl, sec-butyl, or t-butyl. These embodiments of R^{6d} can be combined with any of the embodiments described herein for R^{6a}, R^{6b}, and X⁶.

In some embodiments, the compound, or the pharmaceutically acceptable salt thereof, is the compound of Formula (I), (Ia), (Ia1), (Ib), (Ib1), (Ic), or (Ic1) wherein R^{6a} is H, methyl, ethyl, n-propyl, isopropyl, n-butyl, isobutyl, —CD₃,

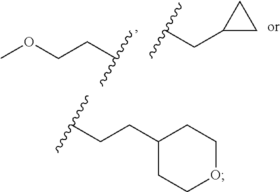

R^{6b} is H; and
R^{6d} is H, methyl, ethyl, n-propyl, isopropyl, —CD₃, or

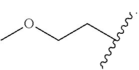 [SMILES: *CCOC]

These embodiments of R^{6a}, R^{6b} and R^{6d} can be combined with any of the embodiments described herein for X⁶.

In some embodiments, the compound, or the pharmaceutically acceptable salt thereof, is the compound of Formula (I), (Ia), (Ia1), (Ib), (Ib1), (Ic), or (Ic1) wherein
R^{6a} is H, methyl, ethyl, n-propyl, isobutyl, —CD₃,
   or

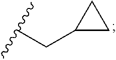 [SMILES: *CC1CC1]

R^{6b} is H; and
R^{6d} is H, methyl, isopropyl, or —CD₃. These embodiments of R^{6a}, R^{6b} and R^{6d} can be combined with any of the embodiments described herein for X⁶.

In some embodiments, the compound, or the pharmaceutically acceptable salt thereof, is the compound of Formula (I), (Ia) or (Ia1) wherein X⁶ is

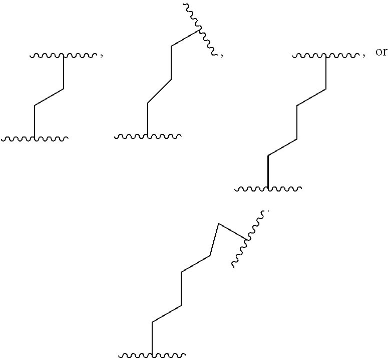

These embodiments of X⁶ can be combined with any of the embodiments described herein for R^{6a}, R^{6b}, R^{6d}, and X⁹.

In some embodiments, the compound, or the pharmaceutically acceptable salt thereof, is the compound of Formula (I), (Ia), or (Ia1) wherein X⁶ is

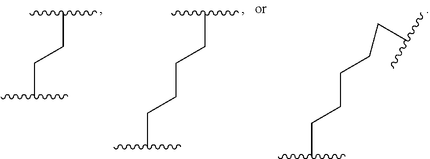

These embodiments of X⁶ can be combined with any of the embodiments described herein for R^{6a}, R^{6b}, R^{6d}, and X⁹.

In some embodiments, the compound, or the pharmaceutically acceptable salt thereof, is the compound of Formula (I), (Ia), or (Ia1) wherein X⁶ is

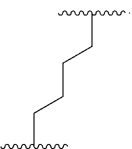 [SMILES: *CCCC*]

These embodiments of X⁶ can be combined with any of the embodiments described herein for R^{6a}, R^{6b}, R^{6d}, and X⁹.

The embodiments described herein for X⁶, R^{6a}, R^{6b} and R^{6d} can be present in any combination. In addition, the embodiments described herein for residue 6 can be present in combination with any of the embodiments described herein for residues 3, 4, 5, 7, 8, and 9. For example, any of the embodiments of X⁶, R^{6a}, R^{6b} and R^{6d} as described herein, can be combined with any of the embodiments described herein for R³, R^{4a}, R^{4b}, R^{4c}, R^{5a}, R^{5b}, R^{5c}, R^{7a}, R^{7b}, R^{7c}, R^{8a}, R^{8b}, R^{8d}, R^{8e}, ring B, m8, R^{8f}, X⁹, R^{9a}, R^{9b}, and R^{9c}.

### Residue 7

In some embodiments, the compound, or the pharmaceutically acceptable salt thereof, is the compound of Formula (I), (Ia), (Ia1), (Ib) or (Ib1) wherein
R^{7a} is H; and
R^{7b} and R^{7c} are each independently H, C₁₋₈ alkyl, or C₁₋₄ alkyl-C₃₋₆ cycloalkyl.

In some embodiments, the compound, or the pharmaceutically acceptable salt thereof, is the compound of Formula (I), (Ia), (Ia1), (Ib) or (Ib1) wherein
R^{7a} is H;
R^{7b} is H; and
R^{7c} is isobutyl, and

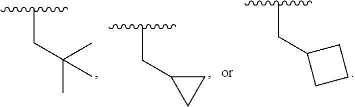

In some embodiments, the compound, or the pharmaceutically acceptable salt thereof, is the compound of Formula (I), (Ia), (Ia1), (Ib) or (Ib1) wherein
R^{7a} is H;
R^{7b} is H; and
R^{7c} is isobutyl.

In some embodiments, the compound, or the pharmaceutically acceptable salt thereof, is the compound of Formula (I), (Ia), (Ia1), (Ib) or (Ib1) wherein
R^{7a} is H;
R^{7b} is H; and
R^{7c} is isobutyl;

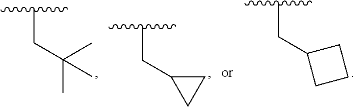

In some embodiments, the compound, or the pharmaceutically acceptable salt thereof, is the compound of Formula (I), (Ia), (Ia1), (Ib), or (Ib1) wherein R^{7a} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia) or (Ib) wherein R^{7a} is H. These embodiments of R^{7a} can be combined with any of the embodiments described herein for R^{7b} and R^{7c}.

In some embodiments, the compound, or the pharmaceutically acceptable salt thereof, is the compound of Formula (I), (Ia), (Ia1), (Ib) or (Ib1) wherein R^{7b} is H. These embodiments of R^{7b} can be combined with any of the embodiments described herein for R^{7a} and R^{7c}.

In some embodiments, the compound, or the pharmaceutically acceptable salt thereof, is the compound Formula (I), (Ia), (Ia1), (Ib) or (Ib1) wherein R^{7c} is isobutyl. In some embodiments, the compound, or the pharmaceutically acceptable salt thereof, is the compound of Formula (I), (Ia), (Ia1), (Ib) or (Ib1) wherein R^{7c} is

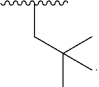 [SMILES: *CC(C)(C)C]

These embodiments of R^{7c} can be combined with any of the embodiments described herein for R^{7a} and R^{7b}.

In some embodiments, the compound, or the pharmaceutically acceptable salt thereof, is the compound of Formula (I), (Ia), (Ia1), (Ib) or (Ib1) wherein R^{7c} is

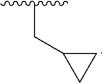 [SMILES: *CC1CC1]

These embodiments of R^{7c} can be combined with any of the embodiments described herein for R^{7a} and R^{7b}.

In some embodiments, the compound, or the pharmaceutically acceptable salt thereof, is the compound of Formula (I), (Ia), (Ia1), (Ib) or (Ib1) wherein R^{7c} is

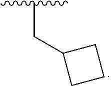 [SMILES: *CC1CCC1]

These embodiments of R^{7c} can be combined with any of the embodiments described herein for R^{7a} and R^{7b}.

The embodiments described herein for R^{7a}, R^{7b} and R^{7c} can be present in any combination. In addition, the embodiments described herein for residue 7 can be present in combination with any of the embodiments described herein for residues 3, 4, 5, 6, 8, and 9. For example, any of the embodiments of R^{7a}, R^{7b} and R^{7c} as described herein, can be combined with any of the embodiments described herein for R³, R^{4a}, R^{4b}, R^{4c}, R^{5a}, R^{5b}, R^{5c}, X⁶, R^{6a}, R^{6b}, R^{6d}, R^{8a}, R^{8b}, R^{8d}, R^{8e}, ring B, m8, R^{8f}, X⁹, R^{9a}, R^{9b}, and R^{9c}.

### Residue 8

In some embodiments, the compound, or the pharmaceutically acceptable salt thereof, is the compound of Formula (I) wherein ring B is phenyl. In some embodiments, the compound, or the pharmaceutically acceptable salt thereof, is the compound of Formula (I) having the structure of Formula (Ia):

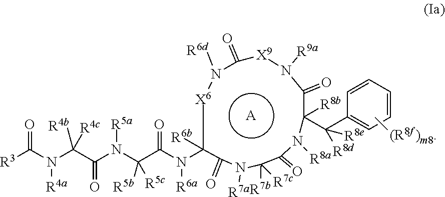

R³, R^{4a}, R^{4b}, R^{4c}, R^{5a}, R^{5b}, R^{5c}, X⁶, R^{6a}, R^{6b}, R^{6d}, R^{7a}, R^{7b}, R^{7c}, R^{8a}, R^{8b}, R^{8d}, R^{8e}, ring B, m8, R^{8f}, X⁹, R^{9a}, R^{9b}, and R^{9c} can each independently be as defined for any embodiment of Formula (Ia) as described herein.

In some embodiments, the compound, or the pharmaceutically acceptable salt thereof, is the compound of Formula (I) having the structure of Formula (Ia1):

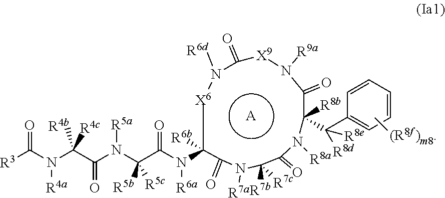

R³, R^{4a}, R^{4b}, R^{4c}, R^{5a}, R^{5b}, R^{5c}, X⁶, R^{6a}, R^{6b}, R^{6d}, R^{7a}, R^{7b}, R^{7c}, R^{8a}, R^{8b}, R^{8d}, R^{8e}, ring B, m8, R^{8f}, X⁹, R^{9a}, R^{9b}, and R^{9c} can each independently be as defined for any embodiment of Formula (Ia1) as described herein.

In some embodiments, the compound, or the pharmaceutically acceptable salt thereof, is the compound of Formula (I) wherein ring B is a heteroaryl having 5 to 12 ring members and 1 to 6 heteroatoms, each heteroatom is N. These embodiments of ring B can be combined with any of the embodiments described herein for R^{8a}, R^{8b}, R^{8d}, R^{8e}, m8 and R^{8f}.

In some embodiments, the compound, or the pharmaceutically acceptable salt thereof, is the compound of Formula (I) wherein ring B is a heteroaryl having 5 to 6 ring members and 1 to 3 heteroatoms each independently N, O or S. These embodiments of ring B can be combined with any of the embodiments described herein for R^{8a}, R^{8b}, R^{8d}, R^{8e}, m8 and R^{8f}.

In some embodiments, the compound, or the pharmaceutically acceptable salt thereof, is the compound of Formula (I) wherein ring B is a heteroaryl having 5 to 6 ring members and 1 to 3 heteroatoms, each heteroatom is N. These embodiments of ring B can be combined with any of the embodiments described herein for R^{8a}, R^{8b}, R^{8d}, R^{8e}, m8 and R^{8f}.

In some embodiments, the compound, or the pharmaceutically acceptable salt thereof, is the compound of Formula (I) wherein ring B is pyridyl or thiophenyl. These embodiments of ring B can be combined with any of the embodiments described herein for R^{8a}, R^{8b}, R^{8d}, R^{8e}, m8 and R^{8f}.

In some embodiments, the compound, or the pharmaceutically acceptable salt thereof, is the compound of Formula (I) wherein ring B is

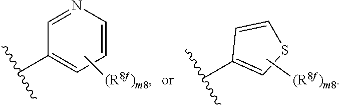

These embodiments of ring B can be combined with any of the embodiments described herein for R^{8a}, R^{8b}, R^{8d}, R^{8e}, m8 and R^{8f}.

The embodiments described herein for ring B can be present in combination with any of the embodiments described herein for the R³, R⁴, R⁵, R⁶, R⁷, R⁸, and R⁹ positions. Accordingly, for any of the embodiments of ring B as described herein R³, R^{4a}, R^{4b}, R^{4c}, R^{5a}, R^{5b}, R^{5c}, X⁶, R^{6a}, R^{6b}, R^{6d}, R^{7a}, R^{7b}, R^{7c}, R^{8a}, R^{8b}, R^{8d}, R^{8e}, m8, R^{8f}, X⁹, R^{9a}, R^{9b}, and R^{9c} can each independently be as defined for any embodiment of Formula (I), (Ia), (Ia1), (Ib), (Ib1), (Ic), or (Ic1) as described herein.

In some embodiments, the compound, or the pharmaceutically acceptable salt thereof, is the compound of Formula (I), (Ia), (Ia1), (Ib), (Ib1), (Ic), or (Ic1) wherein
R^{8a} is C₁₋₄ alkyl, C₁₋₄ deuteroalkyl, C₂₋₆ alkoxyalkyl, or C₁₋₄ alkyl-C₃₋₆ cycloalkyl;
R^{8b}, R^{8d}, and R^{8e} are each independently H;
alternatively R^{8b} and R^{8d} together with the carbons to which each is attached combine to form a C₃₋₆ cycloalkyl;
the subscript m8 is an integer from 0 to 5;
each R^{8f} is independently C₁₋₄ alkyl, C₂₋₄ alkenyl, C₂₋₄ alkynyl, C₁₋₄ alkoxy, C₂₋₈ alkoxyalkyl, halo, C₁₋₄ haloalkyl, C₁₋₄ haloalkoxy, cyano, —NR^{8f1}R^{8f2}, —C(O)NR^{8f1}R^{8f2}, —N(R^{8f1})C(O)R^{8f2}, C₃₋₆ cycloalkyl, —O—C₃₋₆ cycloalkyl, C₁₋₄ alkyl-C₃₋₆ cycloalkyl, —O—C₁₋₄ alkyl-C₃₋₆ cycloalkyl, heterocycloalkyl, —C₁₋₄ alkyl-heterocycloalkyl, phenyl, —O-phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S, wherein each cycloalkyl, heterocycloalkyl, phenyl, and heteroaryl is substituted with 0 to 3 R^{8f3};
each R^{8f1} and R^{8f2} are independently H or C₁₋₄ alkyl; and
each R^{8f3} is independently C₁₋₄ alkyl, —OH, C₁₋₄ alkoxy, halo, C₁₋₄ haloalkyl, C₁₋₄ haloalkoxy, —C(O)C₁₋₄ alkyl, or heterocycloalkyl having 4 to 6 members and 0 to 2 additional heteroatoms each independently N, O or S.
   These embodiments of R^{8a}, R^{8b}, R^{8d}, R^{8e}, m8 and R^{8f} can be combined with any of the embodiments described herein for ring B.

In some embodiments, the compound, or the pharmaceutically acceptable salt thereof, is the compound of Formula (I), (Ia), (Ia1), (Ib), (Ib1), (Ic), or (Ic1) wherein
R^{8a} is C₁₋₄ alkyl, C₁₋₄ deuteroalkyl, or C₁₋₄ alkyl-C₃₋₆ cycloalkyl;
R^{8b}, R^{8d}, and R^{8e} are each independently H;
the subscript m8 is an integer from 0 to 5;
each R^{8f} is independently C₁₋₄ alkyl, C₁₋₄ alkoxy, C₂₋₈ alkoxyalkyl, halo, C₁₋₄ haloalkyl, C₁₋₄ haloalkoxy, cyano, —NR^{8f1}R^{8f2}, C₃₋₆ cycloalkyl, —O—C₃₋₆ cycloalkyl, —O—C₁₋₄ alkyl-C₃₋₆ cycloalkyl, heterocycloalkyl, C₁₋₄ alkyl-heterocycloalkyl, phenyl, —O-phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S, wherein each cycloalkyl, heterocycloalkyl, phenyl, and heteroaryl is substituted with 0 to 3 R^{8f3};
each R^{8f1} and R^{8f2} are each C₁₋₄ alkyl; and
each R^{8f3} is independently C₁₋₄ alkyl, —OH, C₁₋₄ alkoxy, halo, C₁₋₄ haloalkyl, C₁₋₄ haloalkoxy, —C(O)C₁₋₄ alkyl, —O—C₁₋₄ alkyl-C₃₋₆ cycloalkyl, or heterocycloalkyl having 4 to 6 members and 0 to 2 additional heteroatoms each independently N, O or S.
   These embodiments of R^{8a}, R^{8b}, R^{8d}, R^{8e}, m8 and R^{8f} can be combined with any of the embodiments described herein for ring B.

In some embodiments, the compound, or the pharmaceutically acceptable salt thereof, is the compound of Formula (I), (Ia), (Ia1), (Ib), (Ib1), (Ic), or (Ic1) wherein
R^{8a} is methyl, ethyl, n-propyl, n-butyl, —CD₃,

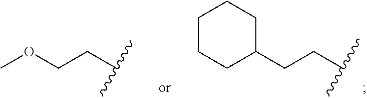

and
R^{8b}, R^{8d} and R^{8e} are each H;
alternatively, R^{8b} and R^{8d} together with the carbons to which each is attached combine to form a cyclopropyl.
   These embodiments of R^{8a}, R^{8b}, R^{8d}, and R^{8e} can be combined with any of the embodiments described herein for m8, R^{8f}, and ring B.

In some embodiments, the compound, or the pharmaceutically acceptable salt thereof, is the compound of Formula (I), (Ia), (Ia1), (Ib), (Ib1), (Ic), or (Ic1) wherein
R^{8a} is methyl, ethyl, n-propyl, n-butyl, —CD₃, or

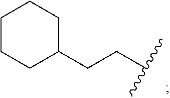 [SMILES: *CCC1CCCCC1]

and
R^{8b}, R^{8d} and R^{8e} are each H.
   These embodiments of R^{8a}, R^{8b}, R^{8d}, and R^{8e} can be combined with any of the embodiments described herein for m8, R^{8f}, and ring B.

In some embodiments, the compound, or the pharmaceutically acceptable salt thereof, is the compound of Formula (I), (Ia), (Ia1), (Ib), (Ib1), (Ic), or (Ic1) wherein
m8 is 0, 1, 2, or 3; and
each R^{8f} is independently methyl, ethynyl, methoxy, fluoro, chloro, bromo, iodo,

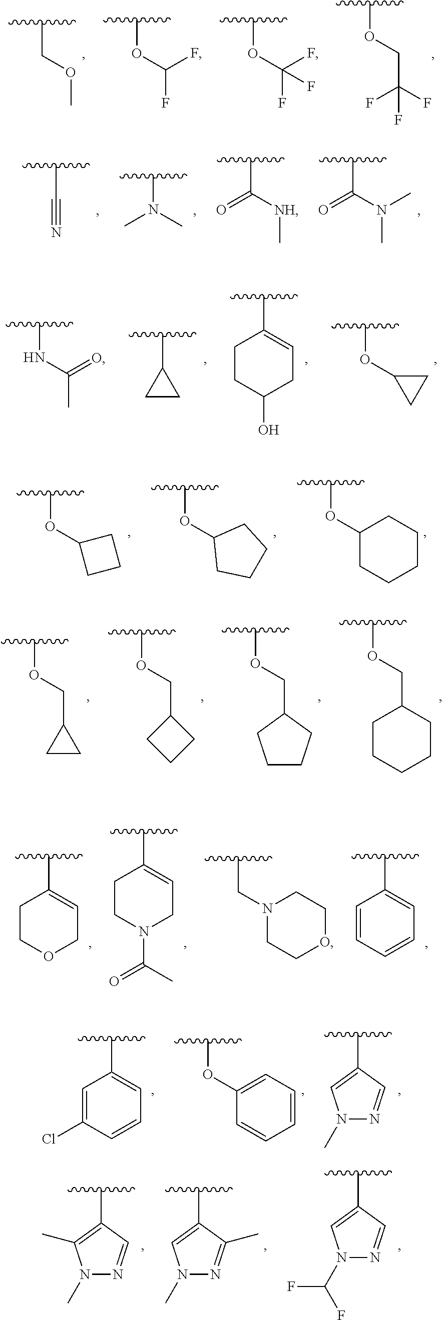

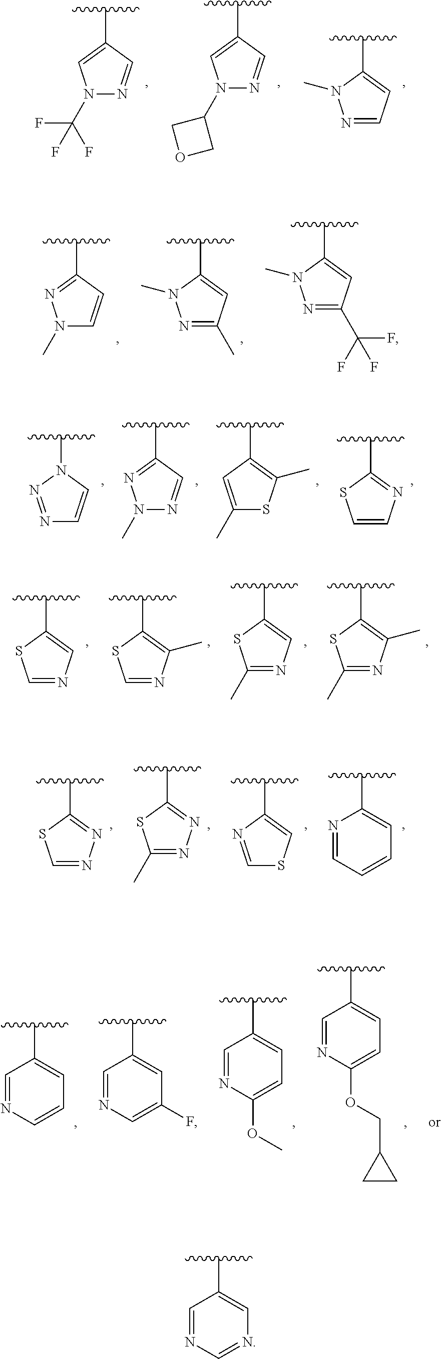

These embodiments of m8 and R^{8f} can be combined with any of the embodiments described herein for R^{8a}, R^{8b}, R^{8d}, R^{8e}, and ring B.

In some embodiments, the compound, or the pharmaceutically acceptable salt thereof, is the compound of Formula (I), (Ia), (Ia1), (Ib), (Ib1), (Ic), or (Ic1) wherein
m8 is 0, 1, 2, or 3; and
each R^{8f} is independently methyl, methoxy, fluoro, chloro, bromo, iodo,

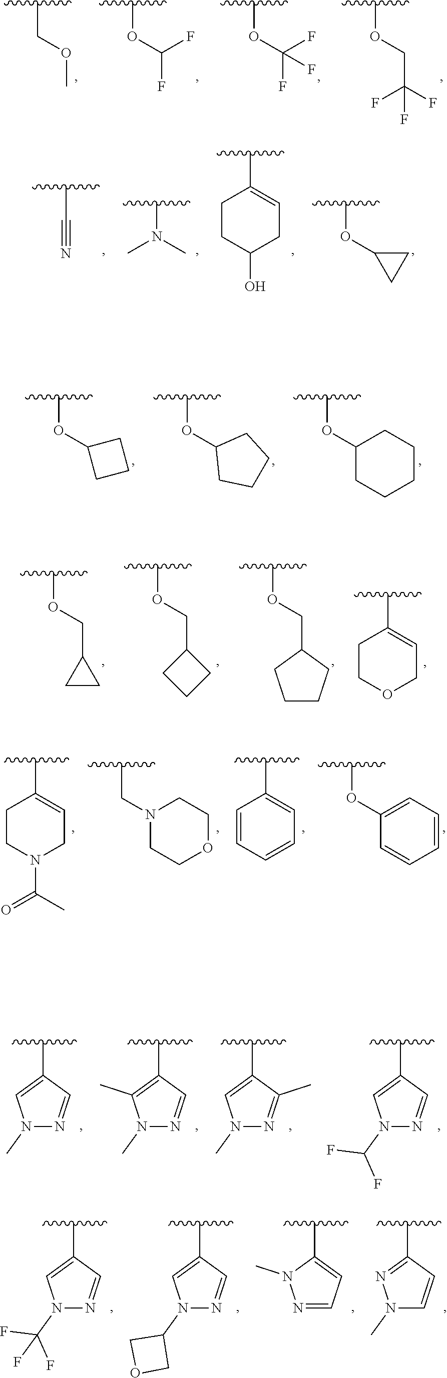

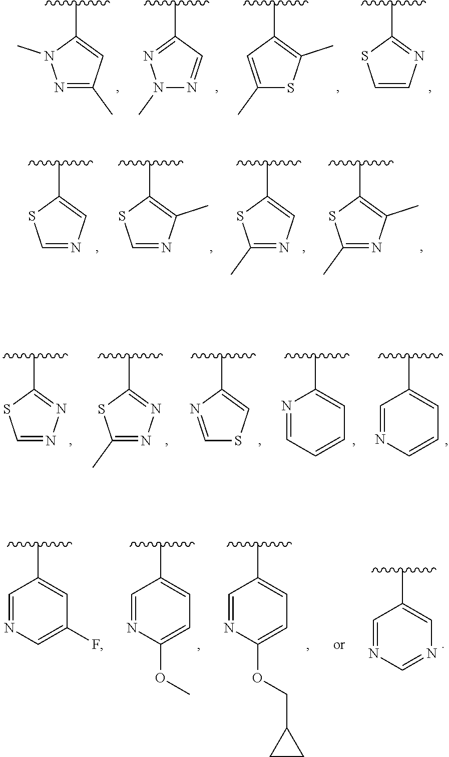

These embodiments of m8 and R^{8f} can be combined with any of the embodiments described herein for R^{8a}, R^{8b}, R^{8d}, R^{8e}, and ring B.

In some embodiments, the compound, or the pharmaceutically acceptable salt thereof, is the compound of Formula (I), (Ia), (Ia1), (Ib), (Ib1), (Ic), or (Ic1) wherein
m8 is 0, 1, 2, or 3; and
each R^{8f} is independently methyl, methoxy, fluoro, chloro, bromo, iodo,

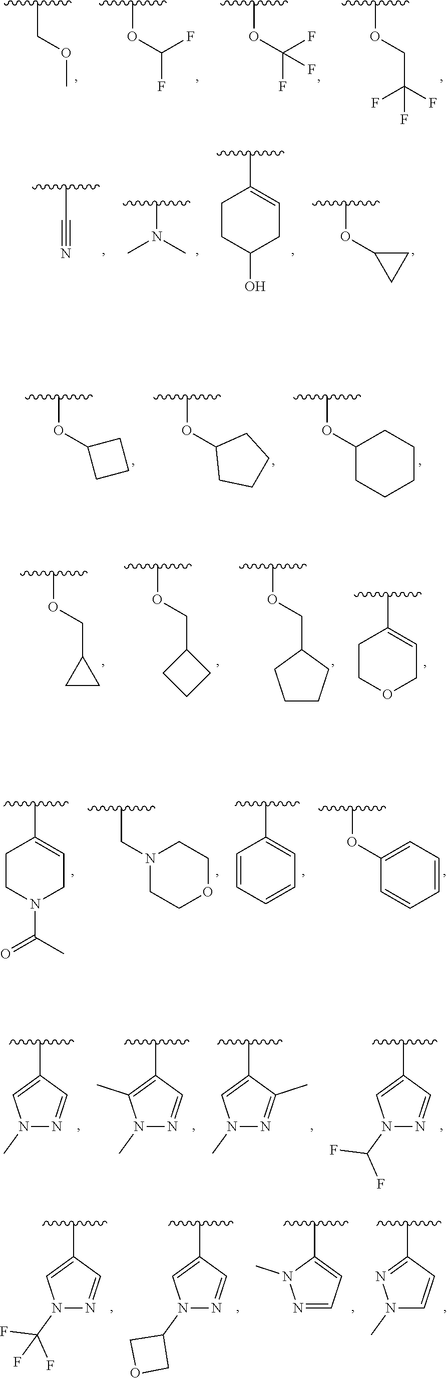

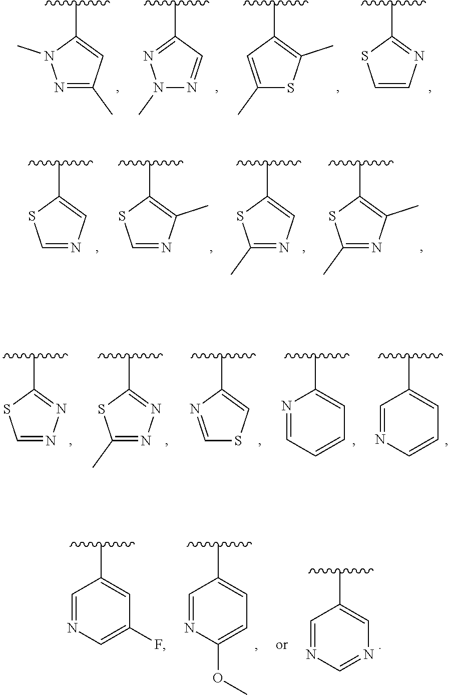

These embodiments of m8 and R^{8f} can be combined with any of the embodiments described herein for R^{8a}, R^{8b}, R^{8d}, R^{8e}, and ring B.

In some embodiments, the compound, or the pharmaceutically acceptable salt thereof, is the compound of Formula (I), (Ia), (Ia1), (Ib), (Ib1), (Ic), or (Ic1) wherein R^{8a} is C₁₋₄ alkyl, C₁₋₄ deuteroalkyl, or C₁₋₄ alkyl-C₃₋₆ cycloalkyl. In some embodiments, the compound, or the pharmaceutically acceptable salt thereof, is the compound of Formula (I), (Ia), (Ia1), (Ib), (Ib1), (Ic), or (Ic1) wherein R^{8a} is C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{8a} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{8a} is C₁₋₄ deuteroalkyl. In some embodiments, the compound, or the pharmaceutically acceptable salt thereof, is the compound of Formula (I), (Ia), (Ia1), (Ib), (Ib1), (Ic), or (Ic1) wherein R^{8a} is C₁₋₄ alkyl-C₃₋₆ cycloalkyl. These embodiments of R^{8a} can be combined with any of the embodiments described herein for R^{8b}, R^{8d}, R^{8e}, m8, R^{8f}, and ring B.

In some embodiments, the compound, or the pharmaceutically acceptable salt thereof, is the compound of Formula (I), (Ia), (Ia1), (Ib), (Ib1), (Ic), or (Ic1) wherein R^{8b} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{8b} is H. These embodiments of R^{8b} can be combined with any of the embodiments described herein for R^{8a}, R^{8d}, R^{8e}, m8, R^{8f}, and ring B.

In some embodiments, the compound, or the pharmaceutically acceptable salt thereof, is the compound of Formula (I), (Ia), (Ia1), (Ib), (Ib1), (Ic), or (Ic1) wherein R^{8d} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{8d} is H. These embodiments of R^{8d} can be combined with any of the embodiments described herein for R^{8a}, R^{8b}, R^{8e}, m8, R^{8f}, and ring B.

In some embodiments, the compound, or the pharmaceutically acceptable salt thereof, is the compound of Formula (I), (Ia), (Ia1), (Ib), (Ib1), (Ic), or (Ic1) wherein R^{8e} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{8e} is H. These embodiments of R^{8e} can be combined with any of the embodiments described herein for R^{8a}, R^{8b}, R^{8d}, m8, R^{8f}, and ring B.

In some embodiments, the compound, or the pharmaceutically acceptable salt thereof, is the compound of Formula (I), (Ia) or (Ib) wherein R^{8b} and R^{8d} together with the carbons to which each is attached combine to form a C₃₋₆ cycloalkyl. These embodiments of R^{8b} and R^{8d} can be combined with any of the embodiments described herein for R^{8a}, R^{8e}, m8, R^{8f}, and ring B.

In some embodiments, the compound, or the pharmaceutically acceptable salt thereof, is the compound of Formula (I), (Ia), (Ia1), (Ib), (Ib1), (Ic), or (Ic1) wherein the subscript m8 is 0. In some embodiments, the compound, or the pharmaceutically acceptable salt thereof, is the compound of Formula (I), (Ia), (Ia1), (Ib), (Ib1), (Ic), or (Ic1) wherein the subscript m8 is 1. In some embodiments, the compound, or the pharmaceutically acceptable salt thereof, is the compound of Formula (I), (Ia), (Ia1), (Ib), (Ib1), (Ic), or (Ic1) wherein the subscript m8 is 2. In some embodiments, the compound, or the pharmaceutically acceptable salt thereof, is the compound of Formula (I), (Ia), (Ia1), (Ib), (Ib1), (Ic), or (Ic1) wherein the subscript m8 is 1 or 2. In some embodiments, the compound, or the pharmaceutically acceptable salt thereof, is the compound of Formula (I), (Ia), (Ia1), (Ib), (Ib1), (Ic), or (Ic1) wherein the subscript m8 is 3. In some embodiments, the compound, or the pharmaceutically acceptable salt thereof, is the compound of Formula (I), (Ia), (Ia1), (Ib), (Ib1), (Ic), or (Ic1) wherein the subscript m8 is 4. In some embodiments, the compound, or the pharmaceutically acceptable salt thereof, is the compound of Formula (I), (Ia), (Ia1), (Ib), (Ib1), (Ic), or (Ic1) wherein the subscript m8 is 5. These embodiments of m8 can be combined with any of the embodiments described herein for R^{8a}, R^{8b}, R^{8d}, R^{8e}, R^{8f}, and ring B.

In some embodiments, the compound, or the pharmaceutically acceptable salt thereof, is the compound of Formula (I), (Ia), (Ia1), (Ib), (Ib1), (Ic), or (Ic1) wherein at least one R^{8f} is C₁₋₄ alkyl, C₁₋₄ alkoxy, C₂₋₈ alkoxyalkyl, halo, C₁₋₄ haloalkyl, C₁₋₄ haloalkoxy, cyano, or —NR^{8f1}R^{8f2}. In some embodiments, the compound, or the pharmaceutically acceptable salt thereof, is the compound of Formula (I), (Ia), (Ia1), (Ib), (Ib1), (Ic), or (Ic1) wherein at least one R^{8f} is C₃₋₆ cycloalkyl, —O—C₃₋₆ cycloalkyl, —O—C₁₋₄ alkyl-C₃₋₆ cycloalkyl, heterocycloalkyl, or C₁₋₄ alkyl-heterocycloalkyl, wherein each heterocycloalkyl has 4 to 6 ring members and 1 to 3 heteroatoms each independently N, O, or S, and wherein each cycloalkyl and heterocycloalkyl is substituted with 0 to 3 R^{8f3}. In some embodiments, the compound, or the pharmaceutically acceptable salt thereof, is the compound of Formula (I), (Ia), (Ia1), (Ib), (Ib1), (Ic), or (Ic1) wherein at least one R^{8f} is phenyl, —O-phenyl, or heteroaryl, wherein each heteroaryl has 5 to 6 ring members and 1 to 3 heteroatoms each independently N, O or S, and wherein each phenyl and heteroaryl is substituted with 0 to 3 R^{8f3}. These embodiments of R^{8f} can be combined with any of the embodiments described herein for R^{8a}, R^{8b}, R^{8d}, R^{8e}, m8, and ring B.

In some embodiments, at least one R^{8f} is C₃₋₆ cycloalkyl, —O—C₃₋₆ cycloalkyl, C₁₋₄ alkyl-C₃₋₆ cycloalkyl, —O—C₁₋₄ alkyl-C₃₋₆ cycloalkyl, heterocycloalkyl, C₁₋₄ alkyl-heterocycloalkyl, phenyl, —O-phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S, wherein each cycloalkyl, heterocycloalkyl, phenyl, and heteroaryl is substituted with 0 to 3 R^{8f3}. In some embodiments, at least one R^{8f} is C₃₋₆ cycloalkyl substituted with 0 to 3 R^{8f3}. In some embodiments, at least one R^{8f} is —O—C₃₋₆ cycloalkyl substituted with 0 to 3 R^{8f3}. In some embodiments, at least one R^{8f} is C₁₋₄ alkyl-C₃₋₆ cycloalkyl substituted with 0 to 3 R^{8f3}. In some embodiments, at least one R^{8f} is —O—C₁₋₄ alkyl-C₃₋₆ cycloalkyl substituted with 0 to 3 R^{8f3}. In some embodiments, at least one R^{8f} is heterocycloalkyl, wherein each heterocycloalkyl has 4 to 6 ring members and 1 to 3 heteroatoms each independently N, O, or S, and each heterocycloalkyl is substituted with 0 to 3 R^{8f3}. In some embodiments, at least one R^{8f} is C₁₋₄ alkyl-heterocycloalkyl, wherein each heterocycloalkyl has 4 to 6 ring members and 1 to 3 heteroatoms each independently N, O, or S, and each heterocycloalkyl is substituted with 0 to 3 R^{8f3}. In some embodiments, at least one R^{8f} is phenyl substituted with 0 to 3 R^{8f3}. In some embodiments, at least one R^{8f} is —O-phenyl substituted with 0 to 3 R^{8f3}. In some embodiments, at least one R^{8f} is heteroaryl, wherein each heteroaryl has 5 to 6 ring members and 1 to 3 heteroatoms each independently N, O or S, and each heterocycloalkyl is substituted with 0 to 3 R^{8f3}. These embodiments of R^{8f} can be combined with any of the embodiments described herein for R^{8a}, R^{8b}, R^{8d}, R^{8e}, m8, and ring B.

In some embodiments, the compound, or the pharmaceutically acceptable salt thereof, is the compound of Formula (I), (Ia), (Ia1), (Ib), (Ib1), (Ic), or (Ic1) wherein each R^{8f3} is C₁₋₄ alkyl, C₁₋₄ alkoxy, halo, C₁₋₄ haloalkyl, C₁₋₄ haloalkoxy, or —O—C₁₋₄ alkyl-C₃₋₆ cycloalkyl. In some embodiments, the compound, or the pharmaceutically acceptable salt thereof, is the compound of Formula (I), (Ia), (Ia1), (Ib), (Ib1), (Ic), or (Ic1) wherein each R^{8f3} is C₁₋₄ alkyl, C₁₋₄ alkoxy, halo, C₁₋₄ haloalkyl, or C₁₋₄ haloalkoxy. In some embodiments, the compound, or the pharmaceutically acceptable salt thereof, is the compound of Formula (I), (Ia), (Ia1), (Ib), (Ib1), (Ic), or (Ic1) wherein each R^{8f3} is C₁₋₄ alkyl, halo, C₁₋₄ haloalkyl. In some embodiments, the compound, or the pharmaceutically acceptable salt thereof, is the compound of Formula (I), (Ia), (Ia1), (Ib), (Ib1), (Ic), or (Ic1) wherein each R^{8f3} is methyl, chloro, or trifluoromethyl. These embodiments of R^{8f3} can be combined with any of the embodiments described herein for R^{8a}, R^{8b}, R^{8d}, R^{8e}, R^{8f}, m8, and ring B.

The embodiments described herein for R^{8a}, R^{8b}, R^{8d}, R^{8e}, m8 and R^{8f} can be present in any combination. In addition, the embodiments described herein for residue 8 can be present in combination with any of the embodiments described herein for residues 3, 4, 5, 6, and 9. For example, any of the embodiments of R^{8a}, R^{8b}, R^{8d}, R^{8e}, m8 and R^{8f} as described herein, can be combined with any of the embodiments described herein for R³, R^{4a}, R^{4b}, R^{4c}, R^{5a}, R^{5b}, R^{5c}, X⁶, R^{6a}, R^{6b}, R^{6d}, R^{7a}, R^{7b}, R^{7c}, X⁹, R^{9a}, R^{9b}, and R^{9c}.

### Residue 9

In some embodiments, the compound, or the pharmaceutically acceptable salt thereof, is the compound of Formula (I), (Ia), or (Ia1) wherein the moiety —C(O)—X⁹—NR^{9a}— is

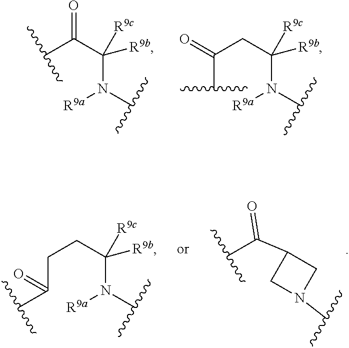

These embodiments of the moiety —C(O)—X⁹—NR^{9a}— can be combined with any of the embodiments described herein for X⁶, R^{9a}, R^{9b}, and R^{9c}.

In some embodiments, the compound, or the pharmaceutically acceptable salt thereof, is the compound of Formula (I), (Ia), or (Ia1) wherein the moiety —C(O)—X⁹—NR^{9a}— is

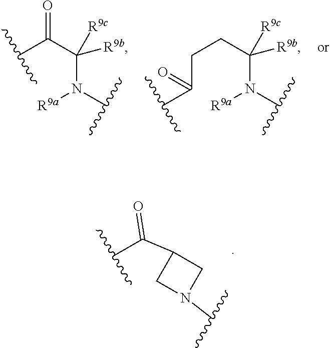

These embodiments of the moiety —C(O)—X⁹—NR^{9a}— can be combined with any of the embodiments described herein for X⁶, R^{9a}, R^{9b}, and R^{9c}.

In some embodiments, the compound, or the pharmaceutically acceptable salt thereof, is the compound of Formula (I), (Ia), or (Ia1) wherein the moiety —C(O)—X⁹—NR^{9a}— is

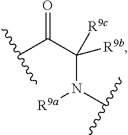

These embodiments of the moiety —C(O)—X⁹—NR^{9a}— can be combined with any of the embodiments described herein for X⁶, R^{9a}, R^{9b}, and R^{9c}.

In some embodiments, the compound, or the pharmaceutically acceptable salt thereof, is the compound of Formula (I), (Ia), (Ia1), (Ib), (Ib1), (Ic), or (Ic1) wherein
R^{9a} is H or C₁₋₄ alkyl;
R^{9b} and R^{9c} are each independently H, C₁₋₆ alkyl, C₁₋₆ alkyl-OH, C₂₋₆ alkoxyalkyl, C₃₋₆ cycloalkyl, C₁₋₄ alkyl-C₃₋₆ cycloalkyl, or C₁₋₄ alkyl-heteroaryl, wherein each heteroaryl has 5 to 6 ring members and from 1 to 3 heteroatoms each independently N, O, or S;
alternatively R^{9b} and R^{9c} together with the carbon to which each is attached combine to form a C₃₋₄ cycloalkyl substituted with 0 to 2 R^{9c2}; or
alternatively R^{9c} and R^{9a} together with the carbon and nitrogen to which each is attached combine to form a heterocycloalkyl having 4 to 6 members and 0 to 2 additional heteroatoms each independently N, O or S, wherein the heterocycloalkyl is substituted with 0 or 2 R^{9c2};
each R^{9c1} is independently halo; and
each R^{9c2} is independently —OH or halo.
   These embodiments of R^{9a}, R^{9b}, and R^{9c} can be combined with any of the embodiments described herein for X⁹.

In some embodiments, the compound, or the pharmaceutically acceptable salt thereof, is the compound of Formula (I), (Ia), (Ia1), (Ib), (Ib1), (Ic), or (Ic1) wherein
R^{9a} is H or C₁₋₄ alkyl;
R^{9b} and R^{9c} are each independently H, C₁₋₆ alkyl, C₂₋₆ alkoxyalkyl, or C₃₋₆ cycloalkyl;
alternatively R^{9b} and R^{9c} together with the carbon to which each is attached combine to form a C₃₋₄ cycloalkyl substituted with 0 to 2 R^{9c2}; or
alternatively R^{9c} and R^{9a} together with the carbon and nitrogen to which each is attached combine to form a heterocycloalkyl having 4 to 6 members and 0 to 2 additional heteroatoms each independently N, O or S, wherein the heterocycloalkyl is substituted with 0 or 2 R^{9c2}; and
each R^{9c2} is independently —OH or halo.
   These embodiments of R^{9a}, R^{9b}, and R^{9c} can be combined with any of the embodiments described herein for X⁹.

In some embodiments, the compound, or the pharmaceutically acceptable salt thereof, is the compound of Formula (I), (Ia), (Ia1), (Ib), (Ib1), (Ic), or (Ic1) wherein R^{9a} is H or C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{9a} is H. In some embodiments, the compound, or the pharmaceutically acceptable salt thereof, is the compound of Formula (I), (Ia), (Ia1), (Ib), (Ib1), (Ic), or (Ic1) wherein R^{9a} is C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{9a} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{9a} is methyl. In some embodiments, the compound, or the pharmaceutically acceptable salt thereof, is the compound of Formula (I), (Ia), (Ia1), (Ib), (Ib1), (Ic), or (Ic1) wherein R^{9a} is ethyl. These embodiments of R^{9a} can be combined with any of the embodiments described herein for R^{9b}, R^{9c}, and X⁹.

In some embodiments, the compound, or the pharmaceutically acceptable salt thereof, is the compound of Formula (I), (Ia), (Ia1), (Ib), (Ib1), (Ic), or (Ic1) wherein R^{9b} is H or C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{9b} is H. In some embodiments, the compound, or the pharmaceutically acceptable salt thereof, is the compound of Formula (I), (Ia), (Ia1), (Ib), (Ib1), (Ic), or (Ic1) wherein R^{9b} is C₁₋₄ alkyl. In some embodiments, the compound, or the pharmaceutically acceptable salt thereof, is the compound of Formula (I), (Ia), (Ia1), (Ib), (Ib1), (Ic), or (Ic1) wherein R^{9b} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{9b} is methyl. In some embodiments, the compound, or the pharmaceutically acceptable salt thereof, is the compound of Formula (I), (Ia), (Ia1), (Ib), (Ib1), (Ic), or (Ic1) wherein R^{9b} is ethyl. These embodiments of R^{9b} can be combined with any of the embodiments described herein for R^{9a}, R^{9c}, and X⁹.

In some embodiments, the compound, or the pharmaceutically acceptable salt thereof, is the compound of Formula (I), (Ia), (Ia1), (Ib), (Ib1), (Ic), or (Ic1) wherein R^{9c} is H, C₁₋₆ alkyl, C₂₋₆ alkoxyalkyl, C₃₋₆ cycloalkyl, C₁₋₄ alkyl-C₃₋₆ cycloalkyl, or C₁₋₄ alkyl-heteroaryl, wherein each heteroaryl has 5 to 6 ring members and from 1 to 3 heteroatoms each independently N, O, or S. In some embodiments, the compound, or the pharmaceutically acceptable salt thereof, is the compound of Formula (I), (Ia), (Ia1), (Ib), (Ib1), (Ic), or (Ic1) wherein R^{9c} is H. In some embodiments, the compound, or the pharmaceutically acceptable salt thereof, is the compound of Formula (I), (Ia), (Ia1), (Ib), (Ib1), (Ic), or (Ic1) wherein R^{9c} is C₁₋₆ alkyl. In some embodiments, the compound, or the pharmaceutically acceptable salt thereof, is the compound of Formula (I), (Ia), (Ia1), (Ib), (Ib1), (Ic), or (Ic1) wherein R^{9c} is methyl, ethyl, n-propyl, iso-propyl, n-butyl, iso-butyl, sec-butyl, or t-butyl. In some embodiments, the compound, or the pharmaceutically acceptable salt thereof, is the compound of Formula (I), (Ia), (Ia1), (Ib), (Ib1), (Ic), or (Ic1) wherein R^{9c} is C₂₋₆ alkoxyalkyl. In some embodiments, the compound, or the pharmaceutically acceptable salt thereof, is the compound of Formula (I), (Ia), (Ia1), (Ib), (Ib1), (Ic), or (Ic1) wherein R^{9c} is C₃₋₆ cycloalkyl. In some embodiments, the compound, or the pharmaceutically acceptable salt thereof, is the compound of Formula (I), (Ia), (Ia1), (Ib), (Ib1), (Ic), or (Ic1) wherein R^{9c} is C₁₋₄ alkyl-heteroaryl. These embodiments of R^{9c} can be combined with any of the embodiments described herein for R^{9a}, R^{9b}, and X⁹.

In some embodiments, the compound, or the pharmaceutically acceptable salt thereof, is the compound of Formula (I), (Ia), (Ia1), (Ib), (Ib1), (Ic), or (Ic1) wherein R^{9b} and R^{9c} together with the carbon to which each is attached combine to form a C₃₋₄ cycloalkyl substituted with 0 to 2 R^{9c2}. In some embodiments, the compound, or the pharmaceutically acceptable salt thereof, is the compound of Formula (I), (Ia), (Ia1), (Ib), (Ib1), (Ic), or (Ic1) wherein the cycloalkyl is substituted with 0 R^{9c2}. In some embodiments, the compound, or the pharmaceutically acceptable salt thereof, is the compound of Formula (I), (Ia), (Ia1), (Ib), (Ib1), (Ic), or (Ic1) wherein the cycloalkyl is substituted with 1 R^{9c2}. In some embodiments, the compound, or the pharmaceutically acceptable salt thereof, is the compound of Formula (I), (Ia), (Ia1), (Ib), (Ib1), (Ic), or (Ic1) wherein the cycloalkyl is substituted with 2 R^{9c2} These embodiments of R^{9b} and R^{9c} can be combined with any of the embodiments described herein for R^{9a} and X⁹.

In some embodiments, the compound, or the pharmaceutically acceptable salt thereof, is the compound of Formula (I), (Ia), (Ia1), (Ib), (Ib1), (Ic), or (Ic1) wherein each R^{9c2} is independently halo or —OH. In some embodiments, the compound, or the pharmaceutically acceptable salt thereof, is the compound of Formula (I), (Ia), (Ia1), (Ib), (Ib1), (Ic), or (Ic1) wherein each R^{9c2} is independently halo. In some embodiments, the compound, or the pharmaceutically acceptable salt thereof, is the compound of Formula (I), (Ia), (Ia1), (Ib), (Ib1), (Ic), or (Ic1) wherein each R^{9c2} is independently —OH. These embodiments of R^{9c2} can be combined with any of the embodiments described herein for R^{9a} combined R^{9b} and R^{9c}, and X⁹.

In some embodiments, the compound, or the pharmaceutically acceptable salt thereof, is the compound of Formula (I), (Ia), (Ia1), (Ib), (Ib1), (Ic), or (Ic1) wherein R^{9c} and R^{9a} together with the carbon and nitrogen to which each is attached combine to form a heterocycloalkyl having 4 to 6 members and 0 to 2 additional heteroatoms each independently N, O or S. In some embodiments, the compound, or the pharmaceutically acceptable salt thereof, is the compound of Formula (I), (Ia), (Ia1), (Ib), (Ib1), (Ic), or (Ic1) wherein the heterocycloalkyl is substituted with 0 or 2 R^{9c2}. In some embodiments, the compound, or the pharmaceutically acceptable salt thereof, is the compound of Formula (I), (Ia), (Ia1), (Ib), (Ib1), (Ic), or (Ic1) wherein the heterocycloalkyl is substituted with 0 R^{9c2}. In some embodiments, the compound, or the pharmaceutically acceptable salt thereof, is the compound of Formula (I), (Ia), (Ia1), (Ib), (Ib1), (Ic), or (Ic1) wherein the heterocycloalkyl is substituted with 1 R^{9c2}. In some embodiments, the compound, or the pharmaceutically acceptable salt thereof, is the compound of Formula (I), (Ia), (Ia1), (Ib), (Ib1), (Ic), or (Ic1) wherein the heterocycloalkyl is substituted with 2 R^{9c2}. These embodiments of R^{9c} and R^{9a} can be combined with any of the embodiments described herein for R^{9b} and X⁹.

In some embodiments, the compound, or the pharmaceutically acceptable salt thereof, is the compound of Formula (I), (Ia), (Ia1), (Ib), (Ib1), (Ic), or (Ic1) wherein each R^{9c2} is independently halo or —OH. In some embodiments, the compound, or the pharmaceutically acceptable salt thereof, is the compound of Formula (I), (Ia), (Ia1), (Ib), (Ib1), (Ic), or (Ic1) wherein each R^{9c2} is independently halo. In some embodiments, the compound, or the pharmaceutically acceptable salt thereof, is the compound of Formula (I), (Ia), (Ia1), (Ib), (Ib1), (Ic), or (Ic1) wherein each R^{9c2} is independently —OH. These embodiments of R^{9c2} can be combined with any of the embodiments described herein for R^{9b}, combined R^{9c} and R^{9a}, and X⁹.

In some embodiments, the compound, or the pharmaceutically acceptable salt thereof, is the compound of Formula (I), (Ia), (Ia1), (Ib), (Ib1), (Ic), or (Ic1) wherein
R^{9a} is H or methyl;
R^{9b} is H, methyl, or ethyl; and
R^{9c} is H, methyl, ethyl, n-propyl, sec-butyl,

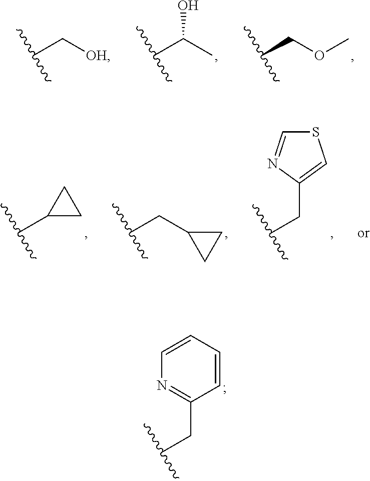

alternatively, R^{9b} and R^{9c} together with the carbon to which they are attached combine to form a C₃₋₄ cycloalkyl substituted with 0 to 2 fluoro groups;
alternatively, R^{9c} and R^{9a} together with the carbon and nitrogen to which each is attached combine to form a heterocycloalkyl having 4- to 6-ring members and 0 additional heteroatoms, the heterocycloalkyl is substituted with 0 or 1 fluoro or —OH groups.
   These embodiments of R^{9a}, R^{9b}, and R^{9c} can be combined with any of the embodiments described herein for X⁹.

In some embodiments, the compound, or the pharmaceutically acceptable salt thereof, is the compound of Formula (I), (Ia), (Ia1), (Ib), (Ib1), (Ic), or (Ic1) wherein
R^{9a} is H or methyl;
R^{9b} is H or methyl; and
R^{9c} is H, methyl, ethyl, n-propyl,

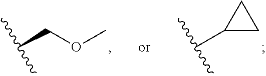

alternatively, R^{9b} and R^{9c} together with the carbon to which they are attached combine to form a C₃₋₄ cycloalkyl substituted with 0 to 2 fluoro groups;
alternatively, R^{9c} and R^{9a} together with the carbon and nitrogen to which each is attached combine to form a heterocycloalkyl having 4- to 6-ring members and 0 additional heteroatoms, the heterocycloalkyl is substituted with 0 or 1 fluoro or —OH groups.
   These embodiments of R^{9a}, R^{9b}, and R^{9c} can be combined with any of the embodiments described herein for X⁹.

The embodiments described herein for X⁹, R^{9a}, R^{9b} and R^{9c} can be present in any combination. In addition, the embodiments described herein for residue 9 can be present in combination with any of the embodiments described herein for residues 3, 4, 5, 6, 7, and 8. For example, any of the embodiments of X⁹, R^{9a}, R^{9b} and R^{9c} as described herein, can be combined with any of the embodiments described herein for R³, R^{4a}, R^{4b}, R^{4c}, R^{5a}, R^{5b}, R^{5c}, X⁶, R^{6a}, R^{6b}, R^{6d}, R^{7a}, R^{7b}, R^{7c}, R^{8a}, R^{8b}, R^{8d}, R^{8e}, ring B, m8 and R^{8f}.

In some embodiments, the compound, or the pharmaceutically acceptable salt thereof, is the compound of Formula (I), (Ia), or (Ia1) wherein
X⁶ is

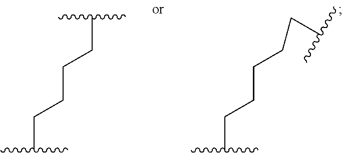 [SMILES: *CCCC* ; *CCCCC*]

and
the moiety —C(O)—X⁹—NR^{9a}— is

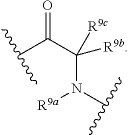

For the above embodiment, R³, R^{4a}, R^{4b}, R^{4c}, R^{5a}, R^{5b}, R^{5c}, R^{6a}, R^{6b}, R^{6d}, R^{7a}, R^{7b}, R^{7c}, R^{8a}, R^{8b}, R^{8d}, R^{8e}, ring B, m8, R^{8f}, R^{9a}, R^{9b} and R^{9c} can each independently be as defined for any embodiment of Formula (I), (Ia), or (Ia1) as described herein.

In some embodiments, the compound, or the pharmaceutically acceptable salt thereof, is the compound of Formula (I), (Ia), or (Ia1) wherein
X⁶ is

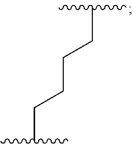 [SMILES: *CCCC*]

and
the moiety —C(O)—X⁹—NR^{9a}— is

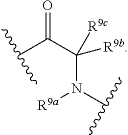

For the above embodiment, R³, R^{4a}, R^{4b}, R^{4c}, R^{5a}, R^{5b}, R^{5c}, R^{6a}, R^{6b}, R^{6d}, R^{7a}, R^{7b}, R^{7c}, R^{8a}, R^{8b}, R^{8d}, R^{8e}, ring B, m8, R^{8f}, R^{9a}, R^{9b} and R^{9c} can each independently be as defined for any embodiment of Formula (I), (Ia), or (Ia1) as described herein.

In some embodiments, the compound, or the pharmaceutically acceptable salt thereof, is the compound of Formula (I) having the structure of Formula (Ib):

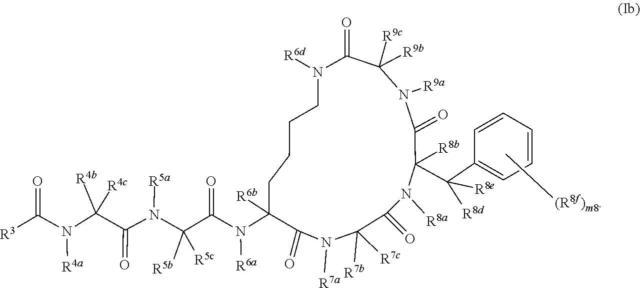

R³, R^{4a}, R^{4b}, R^{4c}, R^{5a}, R^{5b}, R^{5c}, R^{6a}, R^{6b}, R^{6d}, R^{7a}, R^{7b}, R^{7c}, R^{8a}, R^{8b}, R^{8d}, R^{8e}, m8, R^{8f}, R^{9a}, R^{9b}, and R^{9c} can each independently be as defined for any embodiment of Formula (Ib) as described herein.

In some embodiments, the compound, or the pharmaceutically acceptable salt thereof, is the compound of Formula (I) having the structure of Formula (Ib1):

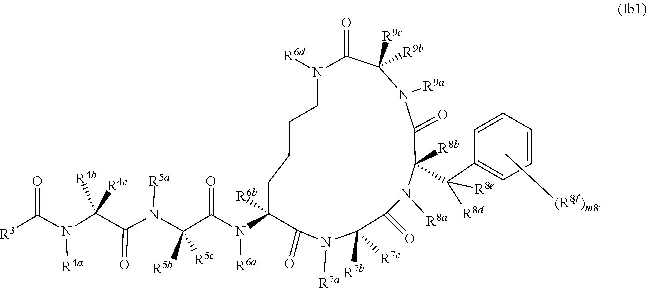

R³, R^{4a}, R^{4b}, R^{4c}, R^{5a}, R^{5b}, R^{5c}, R^{6a}, R^{6b}, R^{6d}, R^{7a}, R^{7b}, R^{7c}, R^{8a}, R^{8b}, R^{8d}, R^{8e}, m8, R^{8f}, R^{9a}, R^{9b}, and R^{9c} can each independently be as defined for any embodiment of Formula (Ib1) as described herein.

In some embodiments, the compound, or the pharmaceutically acceptable salt thereof, is the compound of Formula (I), (Ia), or (Ia1) wherein
X⁶ is

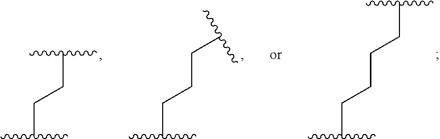

and
the moiety —C(O)—X⁹—NR^{9a}— is

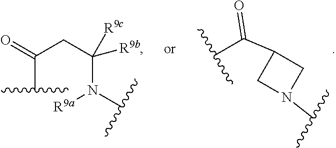

For the above embodiment, R³, R^{4a}, R^{4b}, R^{4c}, R^{5a}, R^{5b}, R^{5c}, R^{6a}, R^{6b}, R^{6d}, R^{7a}, R^{7b}, R^{7c}, R^{8a}, R^{8b}, R^{8d}, R^{8e}, ring B, m8, R^{8f}, R^{9a}, R^{9b} and R^{9c} can each independently be as defined for any embodiment of Formula (I), (Ia), or (Ia1) as described herein.

In some embodiments, the compound, or the pharmaceutically acceptable salt thereof, is the compound of Formula (I), (Ia), or (Ia1) wherein
X⁶ is

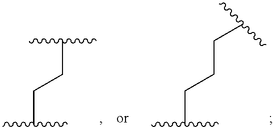

and
the moiety —C(O)—X⁹—NR^{9a}— is

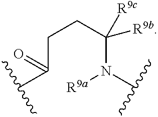

For the above embodiment, R³, R^{4a}, R^{4b}, R^{4c}, R^{5a}, R^{5b}, R^{5c}, R^{6a}, R^{6b}, R^{6d}, R^{7a}, R^{7b}, R^{7c}, R^{8a}, R^{8b}, R^{8d}, R^{8e}, ring B, m8, R^{8f}, R^{9a}, R^{9b} and R^{9c} can each independently be as defined for any embodiment of Formula (I), (Ia), or (Ia1) as described herein.

### Residues 3 to 9

In some embodiments, the compound, or the pharmaceutically acceptable salt thereof, is the compound of Formula (I), (Ia), (Ia1), (Ib), (Ib1), (Ic), and (Ic1) wherein
R³ is

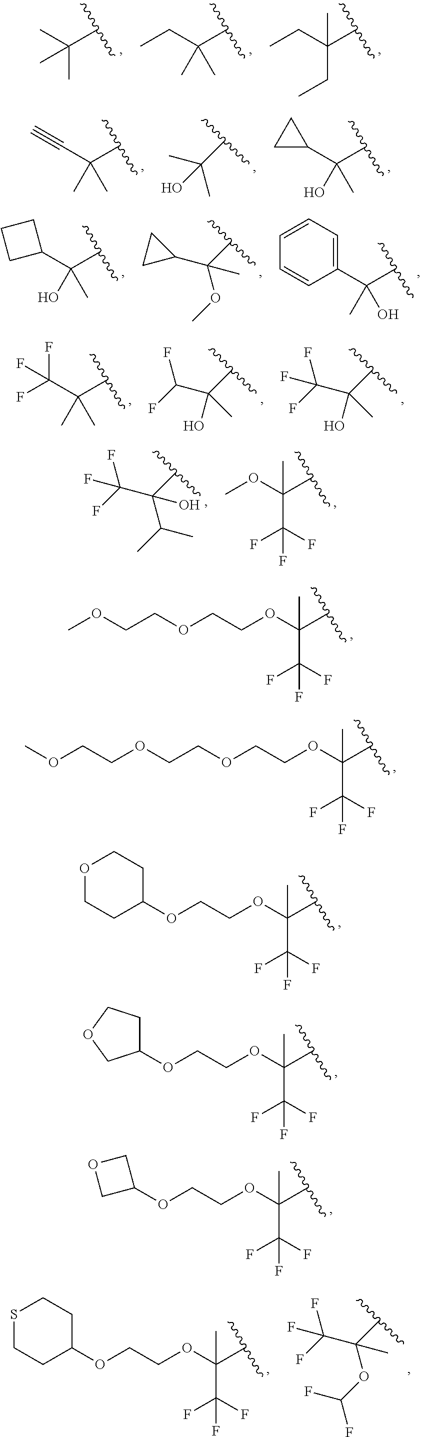

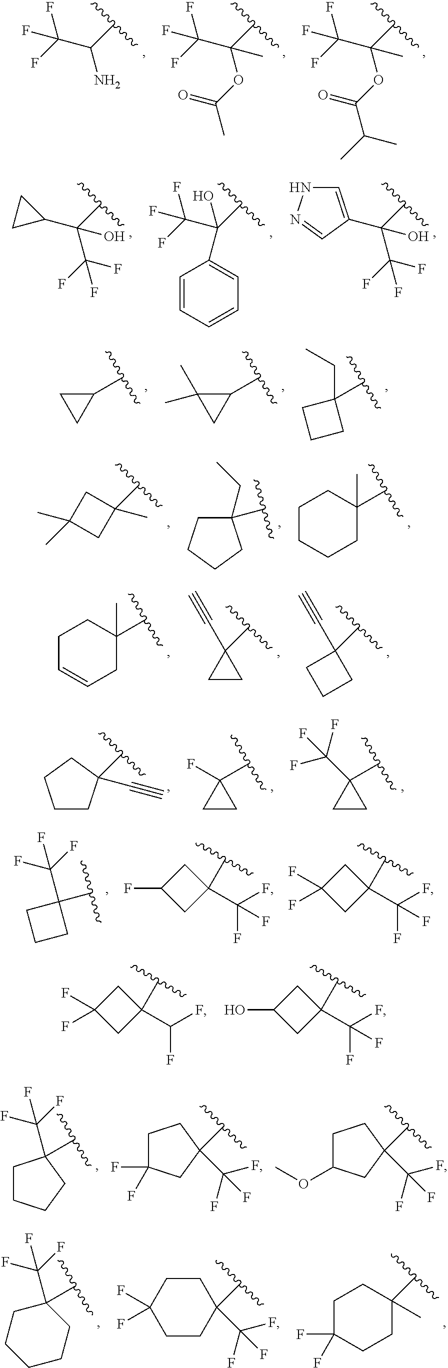

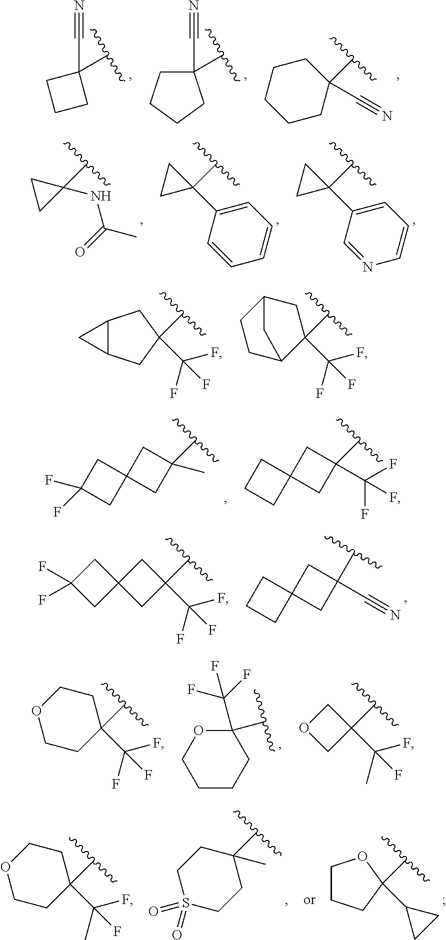

R^{4a} is H or methyl;
R^{4b} is H;
R^{4c} is methyl, ethyl, isopropyl, tert-butyl,

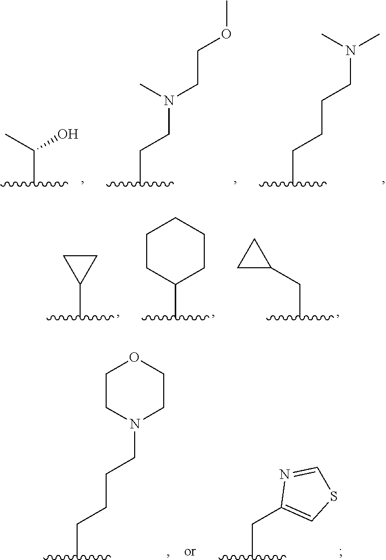

alternatively R^{4c} and R^{4a} together with the carbon and nitrogen to which each is attached combine to form a heterocycloalkyl having 4 to 6 ring members and 0 to 1 additional oxygen, wherein the heterocycloalkyl is substituted with 0 to 2 R^{4a1};
each R^{4a1} is independently methyl, —OH, methoxy, fluoro, or —N(H)S(O)₂CH₃;
alternatively, two R^{4a1} groups on adjacent ring atoms combine to form a phenyl ring substituted with 0 to 2 —OH;
R^{5a} is H;
R^{5b} is H;
R^{5c} is H, methyl, ethyl, n-propyl, isopropyl, n-butyl, sec-butyl, isobutyl, tert-butyl,

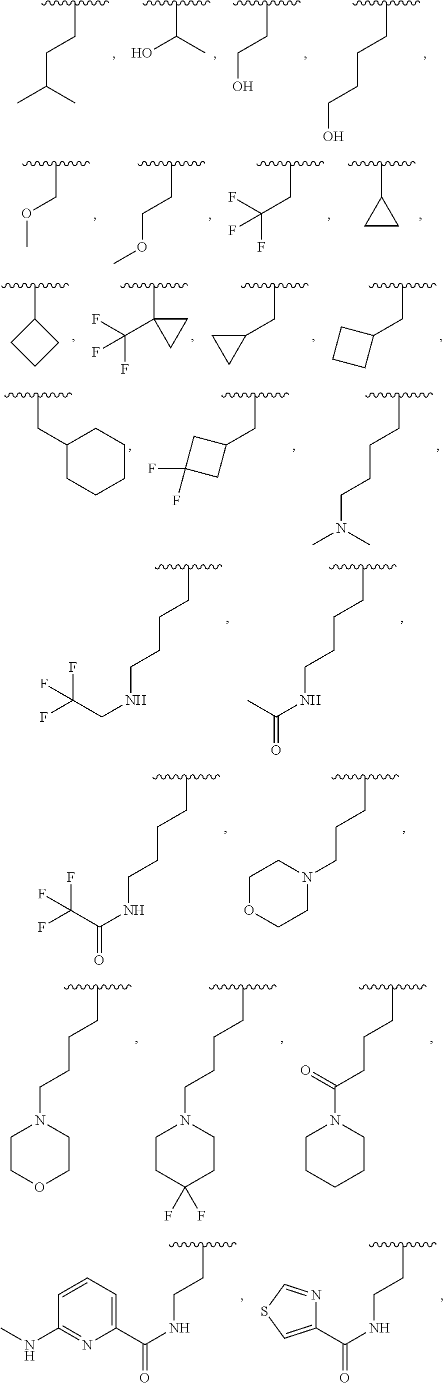

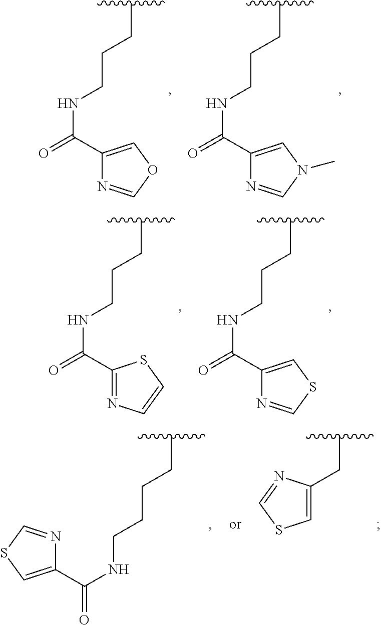

X⁶ is

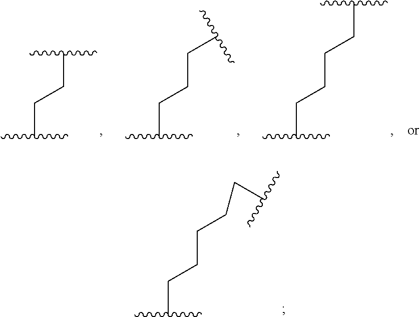

R^{6a} is H, methyl, ethyl, n-propyl, isopropyl, n-butyl, isobutyl, —CD₃,

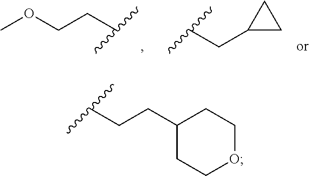

R^{6b} is H;
R^{6d} is H, methyl, ethyl, n-propyl, isopropyl, —CD₃, or

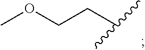 [SMILES: *CCOC]

R^{7a} is H;
R^{7b} is H;
R^{7c} is isobutyl,

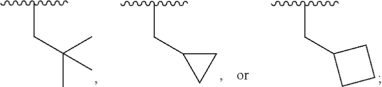

R^{8a} is methyl, ethyl, n-propyl, n-butyl, —CD₃,

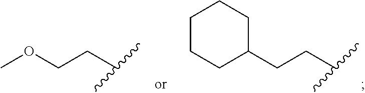

R^{8b}, R^{8d} and R^{8e} are each H;
alternatively, R^{8b} and R^{8d} together with the carbons to which each is attached combine to form a cyclopropyl;
m8 is 0, 1, 2, or 3;
each R^{8f} is independently methyl, ethynyl, methoxy, fluoro, chloro, bromo, iodo,

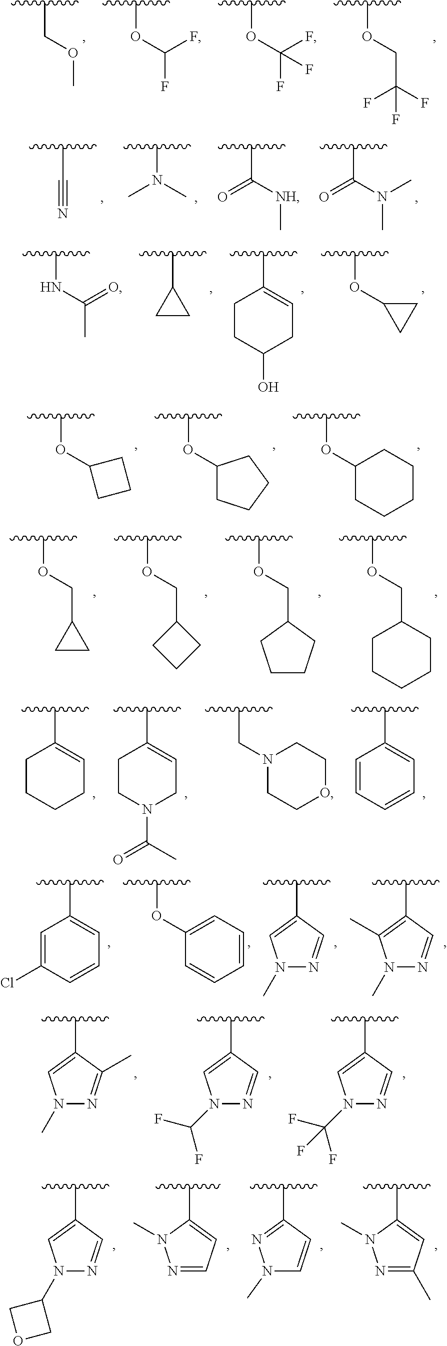

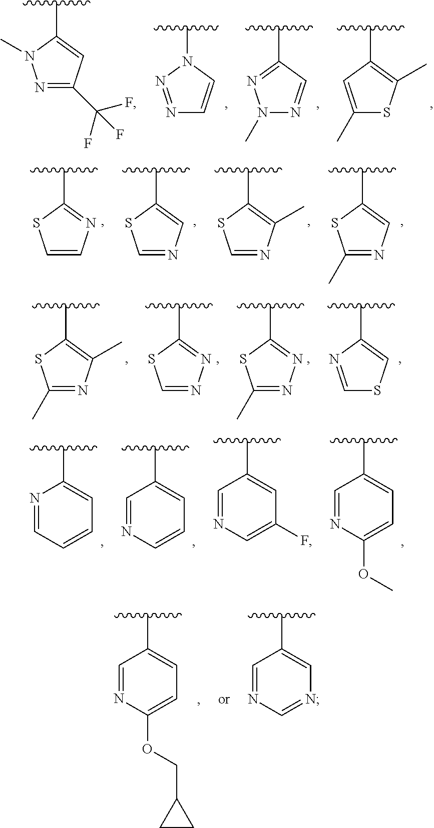

X⁹ is

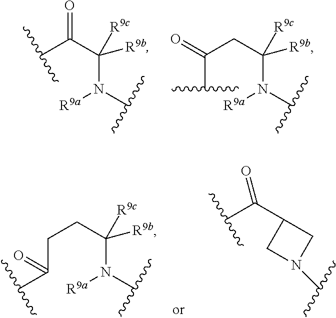

R^{9a} is H or methyl;
R^{9b} is H, methyl, or ethyl; and
R^{9c} is H, methyl, ethyl, n-propyl, sec-butyl,

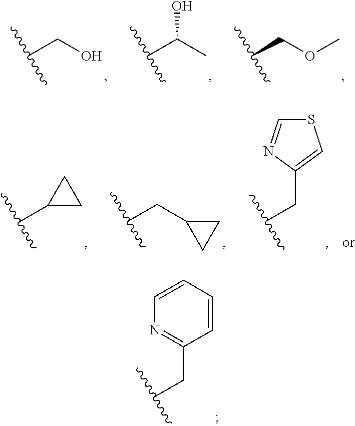

alternatively, R^{9b} and R^{9c} together with the carbon to which they are attached combine to form a C₃₋₄ cycloalkyl substituted with 0 to 2 fluoro groups;
alternatively, R^{9c} and R^{9a} together with the carbon and nitrogen to which each is attached combine to form a heterocycloalkyl having 4- to 6-ring members and 0 additional heteroatoms, the heterocycloalkyl is substituted with 0 or 1 fluoro or —OH groups.

For the above embodiment, R³, R^{4a}, R^{4b}, R^{4c}, R^{5a}, R^{5b}, R^{5c}, R^{6a}, R^{6b}, R^{6d}, R^{7a}, R^{7b}, R^{7c}, R^{8a}, R^{8b}, R^{8d}, R^{8e}, ring B, m8, R^{8f}, R^{9a}, R^{9b} and R^{9c} can each independently be as defined for any embodiment of Formula (I), (Ia), (Ia1), (Ib), (Ib1), (Ic), or (Ic1) as described herein.

In some embodiments, the compound, or the pharmaceutically acceptable salt thereof, is the compound of Formula (I), (Ia), (Ia1), (Ib), (Ib1), (Ic), and (Ic1) wherein
R³ is

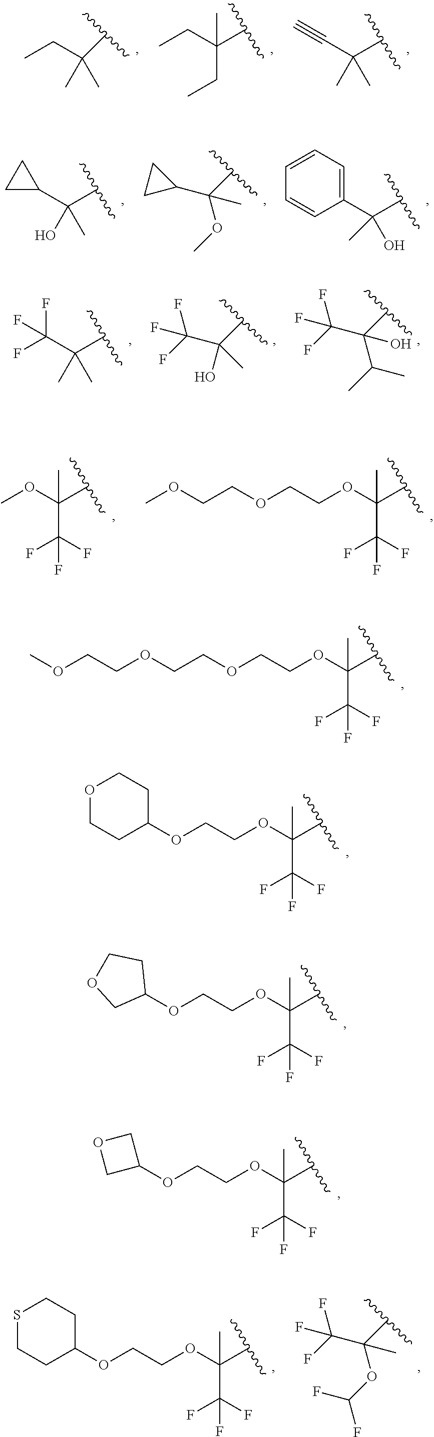

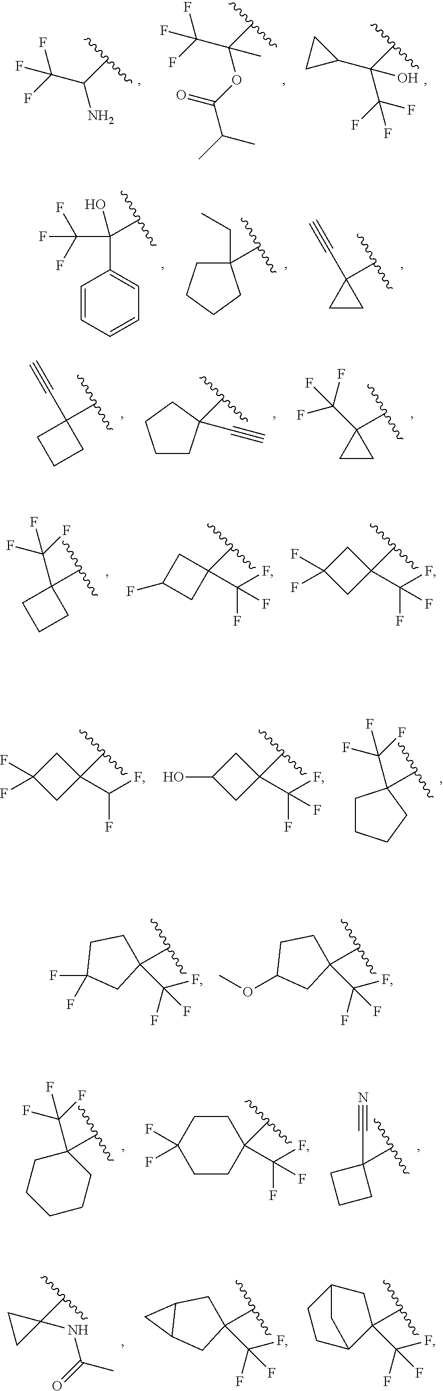

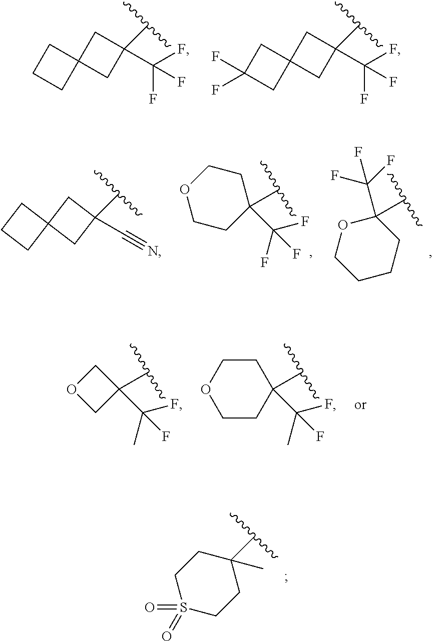

R^{4a} is H or methyl;
R^{4b} is H;
R^{4c} is methyl, ethyl, isopropyl,

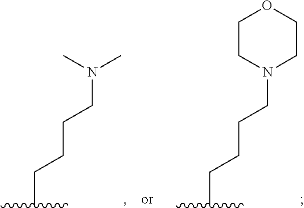

alternatively R^{4c} and R^{4a} together with the carbon and nitrogen to which each is attached combine to form a heterocycloalkyl having 4 to 6 ring members and 0 additional heteroatoms, wherein the heterocycloalkyl is substituted with 0 to 2 R^{4a1};
each R^{4a1} is independently-OH or fluoro;
alternatively, two R^{4a1} groups on adjacent ring atoms combine to form a phenyl ring substituted with 0 to 1 —OH;
R^{5a} is H;
R^{5b} is H;
R^{5c} is H, methyl, ethyl, n-propyl, isopropyl, n-butyl, isobutyl, tert-butyl,

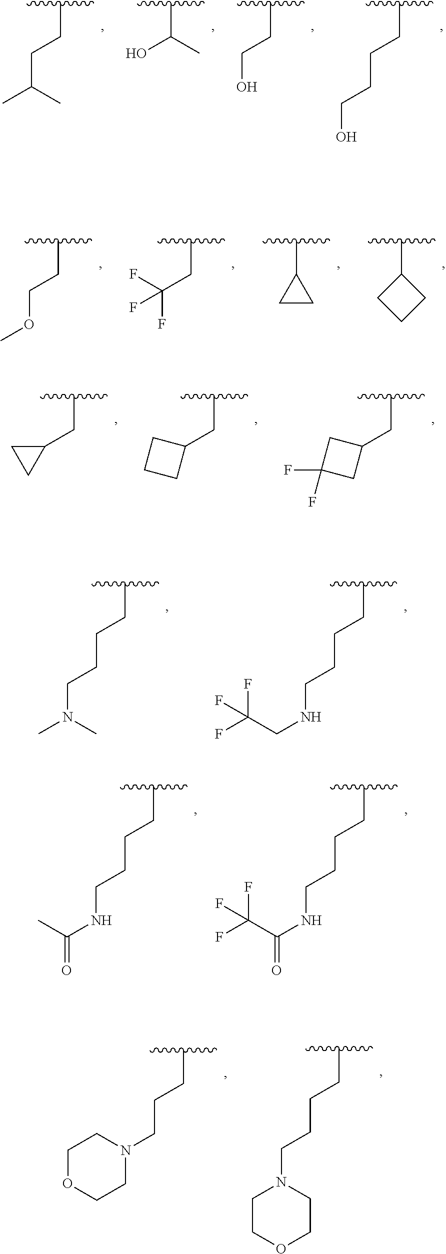

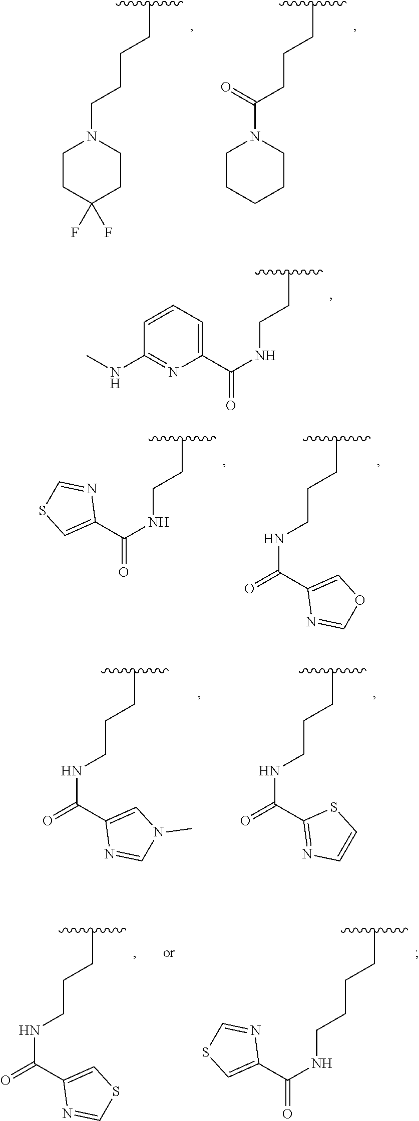

X⁶ is

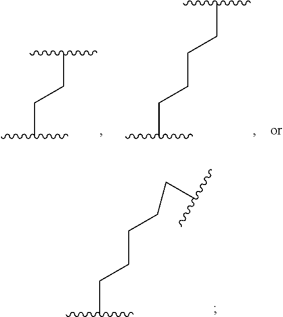

R^{6a} is H, methyl, ethyl, n-propyl, isobutyl, —CD₃,
   or

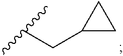 [SMILES: *CC1CC1]

R^{6b} is H;
R^{6d} is H, methyl, isopropyl, or —CD₃,

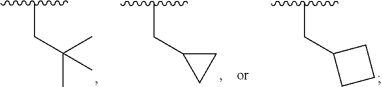

R^{7a} is H;
R^{7b} is H;
R^{7c} is isobutyl,

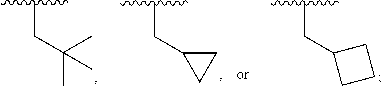

R^{8a} is methyl, ethyl, n-propyl, n-butyl, —CD₃, or

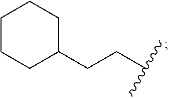 [SMILES: *CCC1CCCCC1]

R^{8b}, R^{8d} and R^{8e} are each H;
m8 is 0, 1, 2, or 3; and
each R^{8f} is independently methyl, methoxy, fluoro, chloro, bromo, iodo,

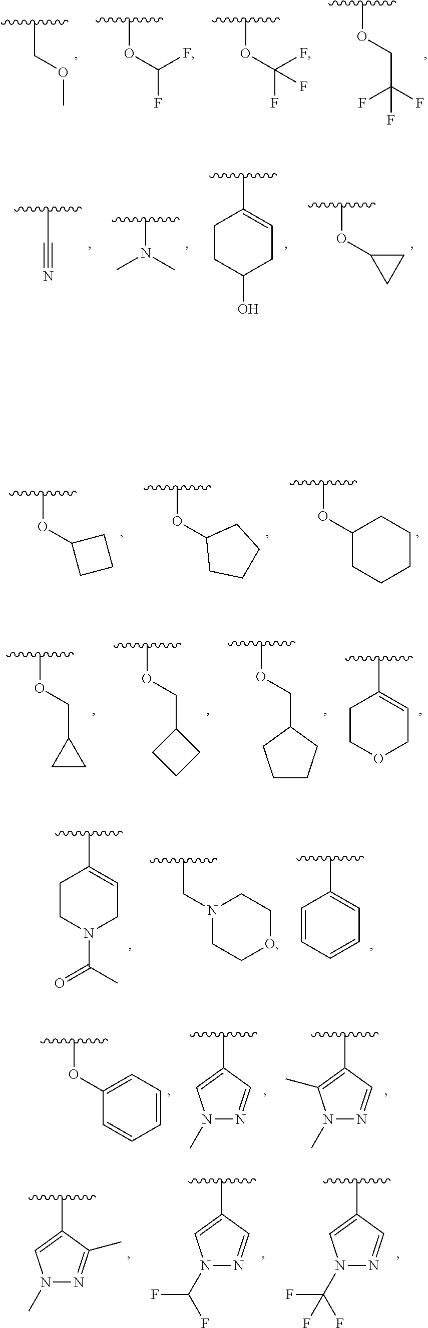

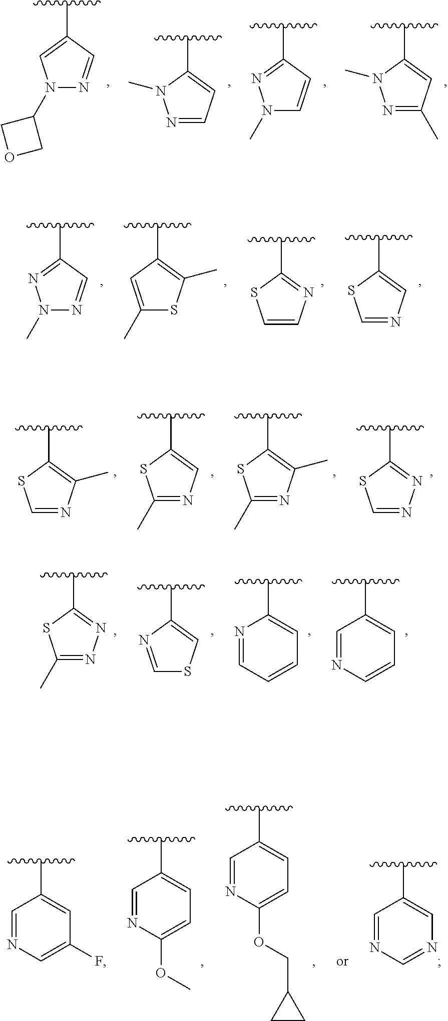

X⁹ is

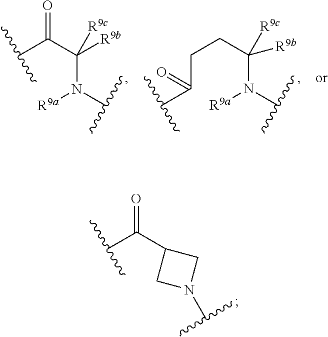

R^{9a} is H or methyl;
R^{9b} is H or methyl; and
R^{9c} is H, methyl, ethyl, n-propyl,

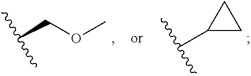

alternatively, R^{9b} and R^{9c} together with the carbon to which they are attached combine to form a C₃₋₄ cycloalkyl substituted with 0 to 2 fluoro groups;
alternatively, R^{9c} and R^{9a} together with the carbon and nitrogen to which each is attached combine to form a heterocycloalkyl having 4- to 6-ring members and 0 additional heteroatoms, the heterocycloalkyl is substituted with 0 or 1 fluoro or —OH groups.

In some embodiments, the compound, or the pharmaceutically acceptable salt thereof, is the compound of Formula (I), (Ia), (Ia1), (Ib), (Ib1), (Ic), and (Ic1) wherein
R³ is

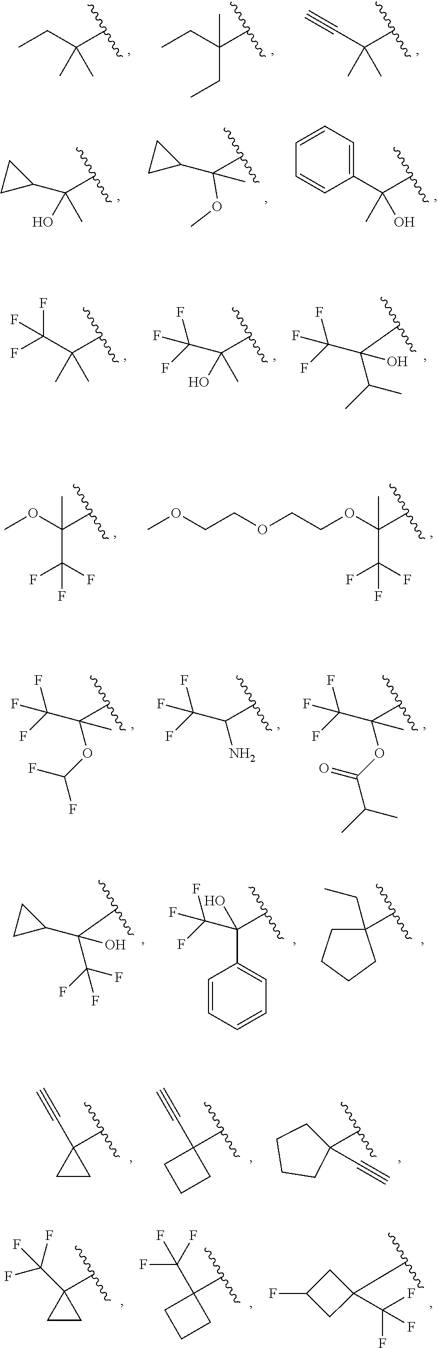

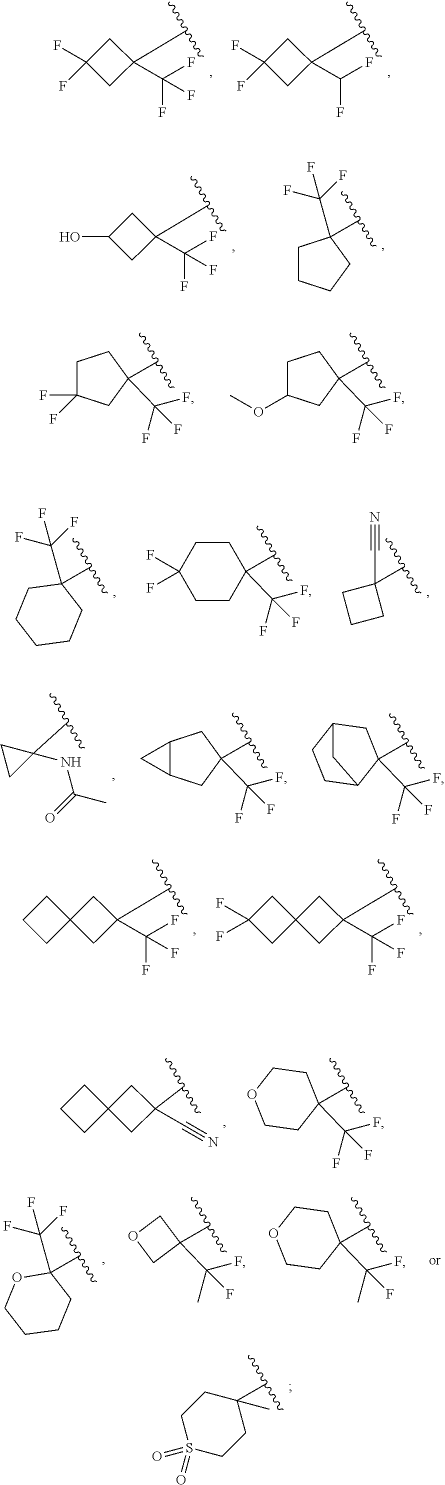

R^{4a} is H or methyl;
R^{4b} is H;
R^{4c} is methyl, ethyl, isopropyl,

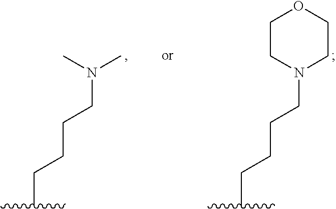

alternatively R^{4c} and R^{4a} together with the carbon and nitrogen to which each is attached combine to form a heterocycloalkyl having 4 to 6 ring members and 0 additional heteroatoms, wherein the heterocycloalkyl is substituted with 0 to 2 R^{4a1};
each R^{4a1} is independently-OH or fluoro;
alternatively, two R^{4a1} groups on adjacent ring atoms combine to form a phenyl ring substituted with 0 to 1 —OH;
R^{5a} is H;
R^{5b} is H;
R^{5c} is H, methyl, ethyl, n-propyl, isopropyl, n-butyl, isobutyl, tert-butyl,

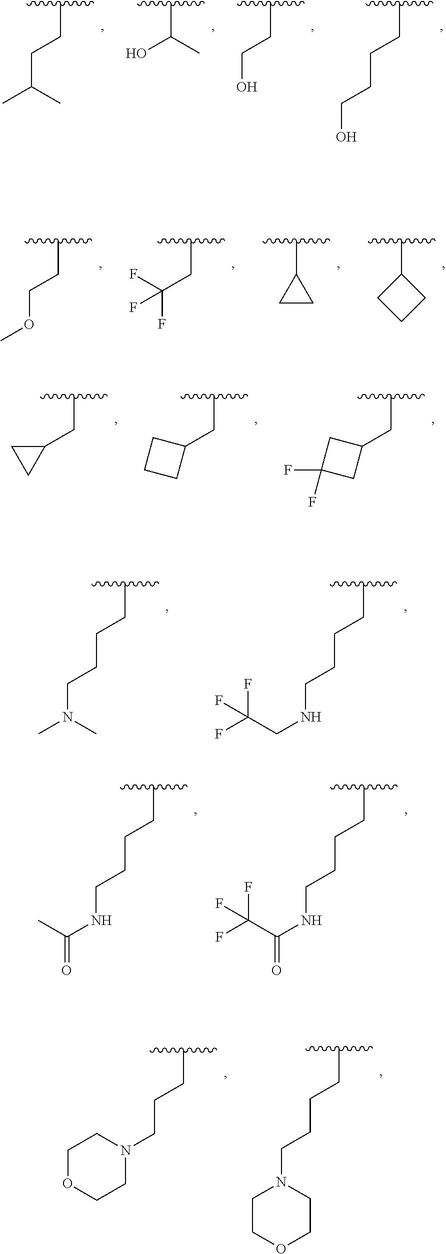

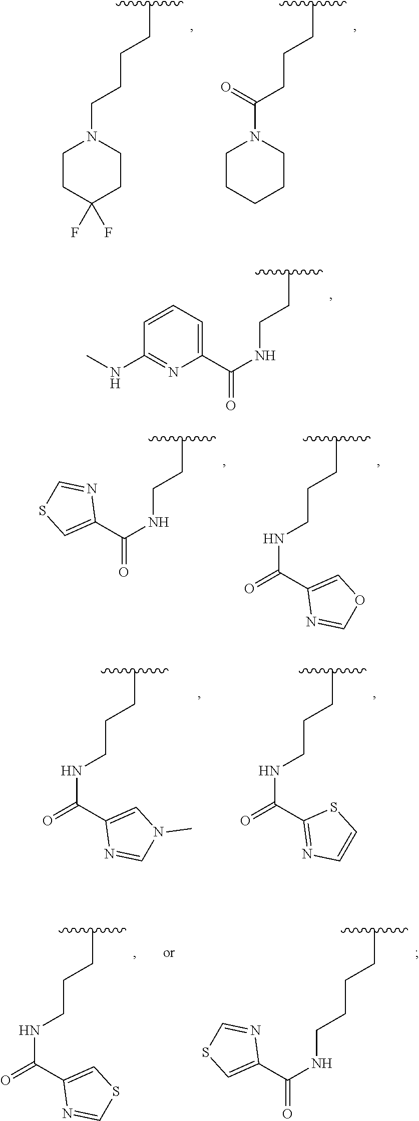

X⁶ is

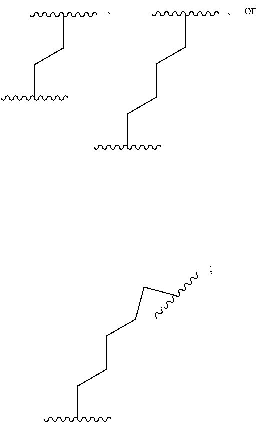

R^{6a} is H, methyl, ethyl, n-propyl, isobutyl, —CD₃,
   or

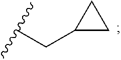 [SMILES: *CC1CC1]

R^{6b} is H;
R^{6d} is H, methyl, isopropyl, or —CD₃,

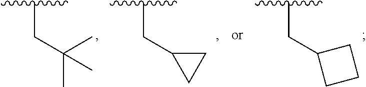

R^{7a} is H;
R^{7b} is H;
R^{7c} is isobutyl,

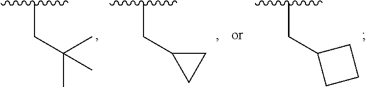

R^{8a} is methyl, ethyl, n-propyl, n-butyl, —CD₃, or

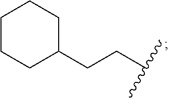 [SMILES: *CCC1CCCCC1]

R^{8b}, R^{8d} and R^{8e} are each H;
m8 is 0, 1, 2, or 3; and
each R^{8f} is independently methyl, methoxy, fluoro, chloro, bromo, iodo,

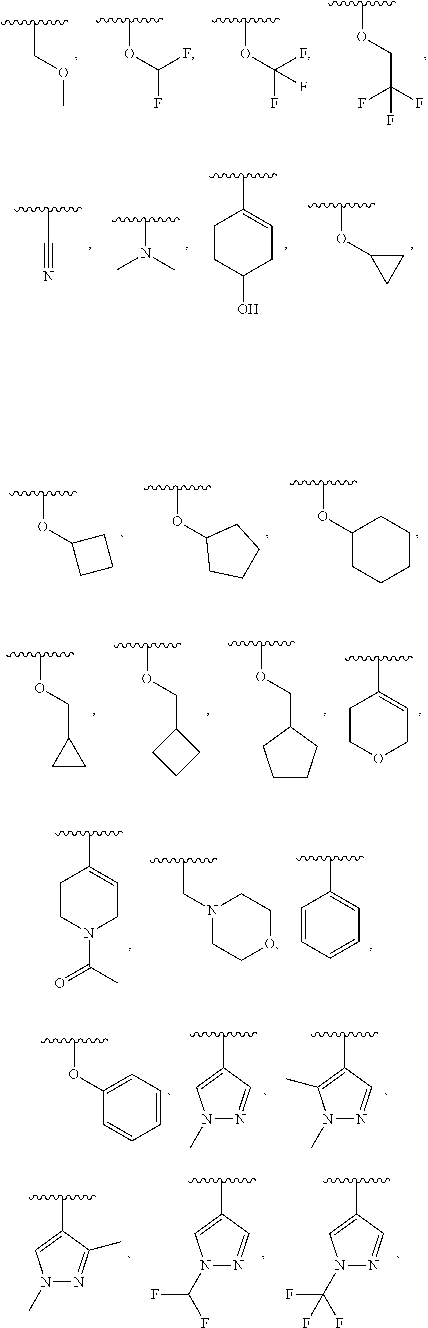

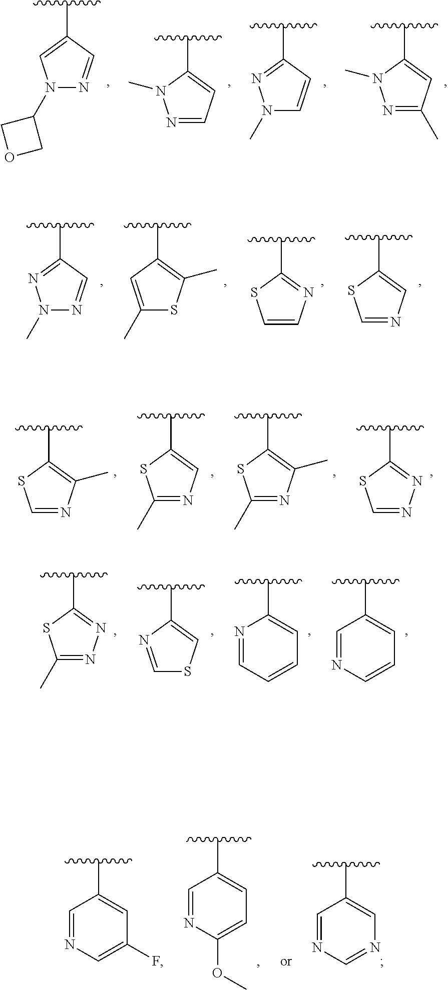

X⁹ is

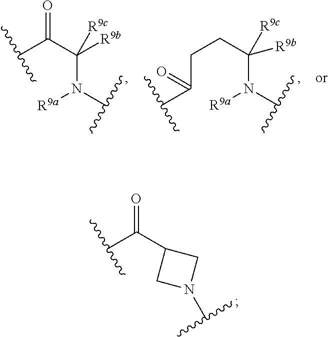

R^{9a} is H or methyl;
R^{9b} is H or methyl; and
R^{9c} is H, methyl, ethyl, n-propyl,

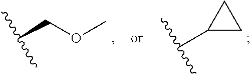

alternatively, R^{9b} and R^{9c} together with the carbon to which they are attached combine to form a C₃₋₄ cycloalkyl substituted with 0 to 2 fluoro groups;
   alternatively, R^{9c} and R^{9a} together with the carbon and nitrogen to which each is attached combine to form a heterocycloalkyl having 4- to 6-ring members and 0 additional heteroatoms, the heterocycloalkyl is substituted with 0 or 1 fluoro or —OH groups.

For the above embodiment, R³, R^{4a}, R^{4b}, R^{4c}, R^{5a}, R^{5b}, R^{5c}, R^{6a}, R^{6b}, R^{6d}, R^{7a}, R^{7b}, R^{7c}, R^{8a}, R^{8b}, R^{8d}, R^{8e}, ring B, m8, R^{8f}, R^{9a}, R^{9b} and R^{9c} can each independently be as defined for any embodiment of Formula (I), (Ia), (Ia1), (Ib), (Ib1), (Ic), or (Ic1) as described herein.

In some embodiments, the compound, or the pharmaceutically acceptable salt thereof, is the compound of Formula (I) having the structure of Formula (Ic):

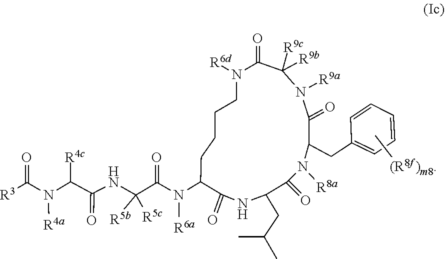

R³, R^{4a}, R^{4c}, R^{5c}, R^{6a}, R^{6d}, R^{8a}, m8, R^{8f}, R^{9a}, R^{9b}, and R^{9c} can each independently be as defined for any embodiment of Formula (Ic) as described herein.

In some embodiments, the compound, or the pharmaceutically acceptable salt thereof, is the compound of Formula (I) having the structure of Formula (Ic1):

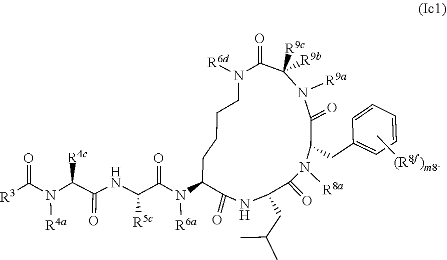

R³, R^{4a}, R^{4c}, R^{5c}, R^{6a}, R^{6d}, R^{8a}, m8, R^{8f}, R^{9a}, R^{9b}, and R^{9c} can each independently be as defined for any embodiment of Formula (Ic1) as described herein.

In some embodiments, the compound, or the pharmaceutically acceptable salt thereof, is the compound of Formula (I), (Ia), (Ia1), (Ib), (Ib1), (Ic), or (Ic1) having the structure of any one of Examples 1-693. In some embodiments, the compound, or the pharmaceutically acceptable salt thereof, is the compound of Formula (I), (Ia), (Ia1), (Ib), (Ib1), (Ic), or (Ic1) having the structure of any one of Examples 1-50. In some embodiments, the compound, or the pharmaceutically acceptable salt thereof, is the compound of Formula (I), (Ia), (Ia1), (Ib), (Ib1), (Ic), or (Ic1) having the structure of any one of Examples 51-100. In some embodiments, the compound, or the pharmaceutically acceptable salt thereof, is the compound of Formula (I), (Ia), (Ia1), (Ib), (Ib1), (Ic), or (Ic1) having the structure of any one of Examples 101-150. In some embodiments, the compound, or the pharmaceutically acceptable salt thereof, is the compound of Formula (I), (Ia), (Ia1), (Ib), (Ib1), (Ic), or (Ic1) having the structure of any one of Examples 151-200. In some embodiments, the compound, or the pharmaceutically acceptable salt thereof, is the compound of Formula (I), (Ia), (Ia1), (Ib), (Ib1), (Ic), or (Ic1) having the structure of any one of Examples 201-250. In some embodiments, the compound, or the pharmaceutically acceptable salt thereof, is the compound of Formula (I), (Ia), (Ia1), (Ib), (Ib1), (Ic), or (Ic1) having the structure of any one of Examples 251-300. In some embodiments, the compound, or the pharmaceutically acceptable salt thereof, is the compound of Formula (I), (Ia), (Ia1), (Ib), (Ib1), (Ic), or (Ic1) having the structure of any one of Examples 301-350. In some embodiments, the compound, or the pharmaceutically acceptable salt thereof, is the compound of Formula (I), (Ia), (Ia1), (Ib), (Ib1), (Ic), or (Ic1) having the structure of any one of Examples 351-400. In some embodiments, the compound, or the pharmaceutically acceptable salt thereof, is the compound of Formula (I), (Ia), (Ia1), (Ib), (Ib1), (Ic), or (Ic1) having the structure of any one of Examples 401-450. In some embodiments, the compound, or the pharmaceutically acceptable salt thereof, is the compound of Formula (I), (Ia), (Ia1), (Ib), (Ib1), (Ic), or (Ic1) having the structure of any one of Examples 451-500. In some embodiments, the compound, or the pharmaceutically acceptable salt thereof, is the compound of Formula (I), (Ia), (Ia1), (Ib), (Ib1), (Ic), or (Ic1) having the structure of any one of Examples 501-550. In some embodiments, the compound, or the pharmaceutically acceptable salt thereof, is the compound of Formula (I), (Ia), (Ia1), (Ib), (Ib1), (Ic), or (Ic1) having the structure of any one of Examples 551-600. In some embodiments, the compound, or the pharmaceutically acceptable salt thereof, is the compound of Formula (I), (Ia), (Ia1), (Ib), (Ib1), (Ic), or (Ic1) having the structure of any one of Examples 601-650. In some embodiments, the compound, or the pharmaceutically acceptable salt thereof, is the compound of Formula (I), (Ia), (Ia1), (Ib), (Ib1), (Ic), or (Ic1) having the structure of any one of Examples 651-693.

The present disclosure includes all tautomers and stereoisomers of the compounds described herein, either in admixture or in pure or substantially pure form. The compounds of Formula (I), (Ia), (Ia1), (Ib), (Ib1), (Ic), or (Ic1) can have asymmetric centers at one or more carbon atoms, and therefore compounds of Formula (I), (Ia), (Ia1), (Ib), (Ib1), (Ic), or (Ic1) can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers, as well as solvates, hydrates, and tautomers are within the scope of the present disclosure. Compounds of Formula (I), (Ia), (Ia1), (Ib), (Ib1), (Ic), or (Ic1) can be prepared using diastereomers, enantiomers or racemic mixtures as starting materials. Furthermore, diastereomer and enantiomer products can be separated by chromatography, fractional crystallization or other methods known to those of skill in the art.

The compounds of Formula (I), (Ia), (Ia1), (Ib), (Ib1), (Ic), or (Ic1) can also be in the salt forms, such as acid or base salts of the compounds of Formula (I), (Ia), (Ia1), (Ib), (Ib1), (Ic), or (Ic1). Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of Formula (I), (Ia), (Ia1), (Ib), (Ib1), (Ic), or (Ic1) are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

The present disclosure also includes isotopically-labeled compounds of Formula (I), (Ia), (Ia1), (Ib), (Ib1), (Ic), or (Ic1), wherein one or more atoms are replaced by one or more atoms having specific atomic mass or mass numbers. Examples of isotopes that can be incorporated into compounds of Formula (I), (Ia), (Ia1), (Ib), (Ib1), (Ic), or (Ic1) include, but are not limited to, isotopes of hydrogen, carbon, nitrogen, oxygen, fluorine, sulfur, and chlorine (such as ²H, ³H, ¹³C, ¹⁴C, ¹⁵N, ¹⁸O, ¹⁷O, ¹⁸F, ³⁵S and ³⁶Cl). Isotopically-labeled compounds of Formula (I), (Ia), (Ia1), (Ib), (Ib1), (Ic), or (Ic1) can be useful in assays of the tissue distribution of the compounds and their prodrugs and metabolites; preferred isotopes for such assays include ³H and ¹⁴C. In addition, in certain circumstances substitution with heavier isotopes, such as deuterium (²H), can provide increased metabolic stability, which offers therapeutic advantages such as increased in vivo half-life or reduced dosage requirements. Isotopically-labeled compounds of Formula (I), (Ia), (Ia1), (Ib), (Ib1), (Ic), or (Ic1) can generally be prepared according to methods known in the art.

### IV. Compositions

The compounds of Formula (I), (Ia), (Ia1), (Ib), (Ib1), (Ic), or (Ic1) described herein are useful in the manufacture of a pharmaceutical composition or a medicament for modulating one or more cyclins (e.g. cyclin A, cyclin B, cycline E). In some embodiments, the present invention provides a pharmaceutical composition comprising a compound of the present invention, and a pharmaceutically acceptable excipient. In some embodiments, a pharmaceutical composition or medicament comprising one or more compounds of Formula (I), (Ia), (Ia1), (Ib), (Ib1), (Ic), or (Ic1) can be administered to a subject for the treatment of a cancer.

Pharmaceutical compositions or medicaments for use in the present disclosure can be formulated by standard techniques or methods well-known in the art of pharmacy using one or more physiologically acceptable carriers or excipients. Suitable pharmaceutical carriers are described herein and in, e.g., “Remington's Pharmaceutical Sciences” by E. W. Martin. Compounds of Formula (I), (Ia), (Ia1), (Ib), (Ib1), (Ic), or (Ic1) and their physiologically acceptable salts and solvates can be formulated for administration by any suitable route, including, but not limited to, orally, topically, nasally, rectally, pulmonary, parenterally (e.g., intravenously, subcutaneously, intramuscularly, etc.), and combinations thereof. In some embodiments, the compounds of Formula (I), (Ia), (Ia1), (Ib), (Ib1), (Ic), or (Ic1) is dissolved in a liquid, for example, water. The most suitable route of administration for a compound of Formula (I), (Ia), (Ia1), (Ib), (Ib1), (Ic), or (Ic1) in any given case will depend, in part, on the nature, severity, and optionally, and the stage of the cancer.

The pharmaceutical compositions or medicaments of the present disclosure can include a compound of Formula (I), (Ia), (Ia1), (Ib), (Ib1), (Ic), or (Ic1) with as an active ingredient and a pharmaceutically acceptable carrier and/or excipient or diluent. Any carrier and/or excipient suitable for the form of preparation desired for administration is contemplated for use with the compounds of Formula (I), (Ia), (Ia1), (Ib), (Ib1), (Ic), or (Ic1) disclosed herein.

In some embodiments, the pharmaceutical compositions or medicaments described herein are suitable for systemic administration. Systemic administration includes enteral administration (e.g., absorption of the compound through the gastrointestinal tract) or parenteral administration (e.g., injection, infusion, or implantation). In some embodiments, the pharmaceutical compositions or medicaments can be administered via a syringe or intravenously. In preferred embodiments, the pharmaceutical compositions or medicaments are injected subcutaneously.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulfate, and/or (f) absorbents, colorants, flavors and sweeteners. In some embodiments, the tablet contains a mixture of hydroxypropyl methylcellulose, polyethyleneglycol 6000 and titanium dioxide. Tablets can be either film coated or enteric coated according to methods known in the art.

Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral, rectal, or vaginal administration is also contemplated.

Pharmaceutical compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art. In certain instances, the compositions can be conveniently delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compounds of Formula (I), (Ia), (Ia1), (Ib), (Ib1), (Ic), or (Ic1) can also be formulated in rectal compositions, for example, suppositories or retention enemas, for example, containing conventional suppository bases, for example, cocoa butter or other glycerides.

The compounds of Formula (I), (Ia), (Ia1), (Ib), (Ib1), (Ic), or (Ic1) set forth herein can be formulated for parenteral administration by injection, for example by bolus injection. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions can be sterilized and/or contain adjuvants, such as preserving, stabilizing, wetting or emulsifying agents, solution promoters, salts for regulating the osmotic pressure and/or buffers. Alternatively, the compound(s) can be in powder form for reconstitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the compound(s).

In some embodiments, the compositions described herein are prepared with a polysaccharide such as chitosan or derivatives thereof (e.g., chitosan succinate, chitosan phthalate, etc.), pectin and derivatives thereof (e.g., amidated pectin, calcium pectinate, etc.), chondroitin and derivatives thereof (e.g., chondroitin sulfate), and alginates.

In some embodiments, the compositions described herein further include a pharmaceutical surfactant. In other embodiments, the compositions further include a cryoprotectant. Non-limiting examples of cryoprotectants include glucose, sucrose, trehalose, lactose, sodium glutamate, PVP, cyclodextrin, 2-hydroxypropyl-13-cyclodextrin (HPI3CD) glycerol, maltose, mannitol, saccharose, and mixtures thereof.

### V. Methods

The present disclosure contemplates the use of the compounds of Formula (I), (Ia), (Ia1), (Ib), (Ib1), (Ic), or (Ic1) described herein in the treatment or prevention of diseases or disorders modulated, at least in part, by one or more cyclins. In some embodiments, the cyclin mediated disease is a proliferative condition or disorder, including cancer. In some embodiments, the present invention provides a method of treating a cancer mediated at least in part by cyclin activity, the method comprising administering to a subject in need there of, a therapeutically effective amount of a compound of the present invention, or a pharmaceutically acceptable salt thereof, or a pharmaceutical composition of the present invention, thereby treating the cancer.

In some embodiments, provided herein are compounds of Formula (I), (Ia), (Ia1), (Ib), (Ib1), (Ic), or (Ic1) for use in therapy.

The present disclosure contemplates the use of the compounds of (I), (Ia), (Ia1), (Ib), (Ib1), (Ic), or (Ic1) described herein in the treatment or prevention of diseases or disorders modulated, at least in part, by cyclin A. In some embodiments, the cyclin A mediated disease is a proliferative condition or disorder, including cancer. In some embodiments, the present invention provides a method of treating a cancer mediated at least in part by cyclin A, the method comprising administering to a subject in need there of, a therapeutically effective amount of a compound of the present invention, or a pharmaceutically acceptable salt thereof, or a pharmaceutical composition of the present invention, thereby treating the cancer.

In some embodiments, provided herein are methods of treating a proliferative condition or disorder mediated at least in part by cyclin A comprising administering a compound of (I), (Ia), (Ia1), (Ib), (Ib1), (Ic), or (Ic1) described herein.

In some embodiments, provided herein are compounds of (I), (Ia), (Ia1), (Ib), (Ib1), (Ic), or (Ic1) for use in a method for treating a proliferative condition or disorder mediated at least in part by cyclin A.

In some embodiments, provided herein are uses of compounds of Formula (I), (Ia), (Ia1), (Ib), (Ib1), (Ic), or (Ic1) for the manufacture of a medicament for the treatment of a proliferative condition or disorder mediated at least in part by cyclin A.

The present disclosure contemplates the use of the compounds of (I), (Ia), (Ia1), (Ib), (Ib1), (Ic), or (Ic1) described herein in the treatment or prevention of diseases or disorders modulated, at least in part, by cyclin B. In some embodiments, the cyclin B mediated disease is a proliferative condition or disorder, including cancer. In some embodiments, the present invention provides a method of treating a cancer mediated at least in part by cyclin B, the method comprising administering to a subject in need there of, a therapeutically effective amount of a compound of the present invention, or a pharmaceutically acceptable salt thereof, or a pharmaceutical composition of the present invention, thereby treating the cancer.

In some embodiments, provided herein are methods of treating a proliferative condition or disorder mediated at least in part by cyclin B comprising administering a compound of (I), (Ia), (Ia1), (Ib), (Ib1), (Ic), or (Ic1) described herein.

In some embodiments, provided herein are compounds of (I), (Ia), (Ia1), (Ib), (Ib1), (Ic), or (Ic1) for use in a method for treating a proliferative condition or disorder mediated at least in part by cyclin B.

In some embodiments, provided herein are uses of compounds of Formula (I), (Ia), (Ia1), (Ib), (Ib1), (Ic), or (Ic1) for the manufacture of a medicament for the treatment of a proliferative condition or disorder mediated at least in part by cyclin B.

The present disclosure contemplates the use of the compounds of (I), (Ia), (Ia1), (Ib), (Ib1), (Ic), or (Ic1) described herein in the treatment or prevention of diseases or disorders modulated, at least in part, by cyclin E. In some embodiments, the cyclin E mediated disease is a proliferative condition or disorder, including cancer. In some embodiments, the present invention provides a method of treating a cancer mediated at least in part by cyclin E, the method comprising administering to a subject in need there of, a therapeutically effective amount of a compound of the present invention, or a pharmaceutically acceptable salt thereof, or a pharmaceutical composition of the present invention, thereby treating the cancer.

In some embodiments, provided herein are methods of treating a proliferative condition or disorder mediated at least in part by cyclin E comprising administering a compound of (I), (Ia), (Ia1), (Ib), (Ib1), (Ic), or (Ic1) described herein.

In some embodiments, provided herein are compounds of (I), (Ia), (Ia1), (Ib), (Ib1), (Ic), or (Ic1) for use in a method for treating a proliferative condition or disorder mediated at least in part by cyclin E.

In some embodiments, provided herein are uses of compounds of Formula (I), (Ia), (Ia1), (Ib), (Ib1), (Ic), or (Ic1) for the manufacture of a medicament for the treatment of a proliferative condition or disorder mediated at least in part by cyclin E.

In some embodiments, the compounds of Formula (I), (Ia), (Ia1), (Ib), (Ib1), (Ic), or (Ic1) described herein can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, bile ducts, heart, lung (e.g., non-small-cell lung carcinoma, small cell lung cancer), pancreas, salivary gland, adrenal gland, thyroid, brain, ganglia, central nervous system (CNS) and peripheral nervous system (PNS), and cancers of the hematopoietic system and the immune system (e.g., spleen or thymus).

The present disclosure also provides methods of treating or preventing other cancer-related diseases, disorders or conditions, including, for example, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis.

In some embodiments, the tumor or cancer is colon cancer, ovarian cancer, breast cancer, melanoma, lung cancer, glioblastoma, or leukemia.

In some embodiments, the tumor or cancer is small cell lung cancer (SCLC).

The use of the term(s) cancer-related diseases, disorders and conditions is meant to refer broadly to conditions that are associated, directly or indirectly, with cancer, and includes, e.g., angiogenesis and precancerous conditions such as dysplasia.

In some embodiments, the cancer is a blood cancer (e.g., leukemia, lymphoma, multiple myeloma).

In some embodiments, the leukemia is acute lymphocytic leukemia, chronic lymphocytic leukemia, acute myelogenous leukemia, chronic myelogenous leukemia, or hairy cell leukemia.

In some embodiments, the lymphoma is non-Hodgkin's lymphoma, Hodgkin's lymphoma, B-cell lymphoma, or Burkitt's lymphoma.

In some embodiments, the cancer is an Rb mutated cancer. In some embodiments, the cancer has a mutation in the Rb/E2F pathway.

### VI. Administration

The present disclosure contemplates the administration of compounds of (I), (Ia), (Ia1), (Ib), (Ib1), (Ic), or (Ic1) and compositions thereof, in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation.

Pharmaceutical compositions comprising compounds of (I), (Ia), (Ia1), (Ib), (Ib1), (Ic), or (Ic1) are preferably in unit dosage form. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Compounds of (I), (Ia), (Ia1), (Ib), (Ib1), (Ic), or (Ic1) or pharmaceutical compositions or medicaments thereof can be administered to a subject diagnosed or suspected of having a disease or disorder mediated at least in part by cyclin A in an amount sufficient to elicit an effective therapeutic response in the subject.

The dosage of compounds administered is dependent on a variety of factors including the subject's body weight, age, individual condition, and/or on the form of administration. The size of the dose will also be determined by the existence, nature, and extent of any adverse effects that accompany the administration of a particular compound in a particular subject. Typically, a dosage of the active compounds is a dosage that is sufficient to achieve the desired effect. Optimal dosing schedules can be calculated from measurements of compound accumulation in the body of a subject. In general, dosage can be given once or more daily, weekly, or monthly. Persons of ordinary skill in the art can easily determine optimum dosages, dosing methodologies, and repetition rates.

In some embodiments, a unit dosage for oral administration of a compound of Formula (I), (Ia), (Ia1), (Ib), (Ib1), (Ic), or (Ic1) described herein to a subject (e.g., a human) of about 50 to about 70 kg may contain between about 1 and about 5,000 mg, about 1 and about 3,000 mg, about 1 and about 2,000 mg, or about 1 to about 1,000 mg of the compound(s).

In some embodiments, a unit dosage for subcutaneous administration of a compound of Formula (I), (Ia), (Ia1), (Ib), (Ib1), (Ic), or (Ic1) described herein to a subject (e.g., human) of about 50 to about 70 kg may contain between about 0.1 and about 500 mg, about 0.5 and about 300 mg, about 0.5 and about 200 mg, about 0.5 and about 100 mg, or about 0.5 and about 50 mg.

The dose can be administered once per day or divided into sub-doses and administered in multiple doses, e.g., twice, three times, or four times per day. However, as will be appreciated by a skilled artisan, depending on the route of administration different amounts can be administered at different times.

In some embodiments, the compounds are administered for about 1 to 31 days, or for about 1 to 12 months. In some embodiments, the compounds are administered for one or more weeks, e.g., 1, 2, 3, 4, 5, 6, 7, 8, 9, 10, 11, 12, 13, 14, or more weeks. In some embodiments, the compounds are administered for one or more months, e.g., 1, 2, 3, 4, 5, 6, 7, 8, 9, 10, 11, 12, or more months.

Optimum dosages, toxicity, and therapeutic efficacy of such compounds may vary depending on the relative potency of individual compounds and can be determined by standard pharmaceutical procedures in experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side-effects can be used, care should be taken to design a delivery system that targets such compounds to the affected site to minimize potential damage to normal cells and, thereby, reduce side-effects.

The dosage of a pharmaceutical composition or medicament of the present disclosure can be monitored and adjusted throughout treatment, depending on severity of symptoms, frequency of recurrence, and/or the physiological response to the therapeutic regimen. Those of skill in the art commonly engage in such adjustments in therapeutic regimens.

Single or multiple administrations of the pharmaceutical compositions or medicaments can be administered depending on the dosage and frequency as required and tolerated by the patient. In any event, the composition or medicament should provide a sufficient quantity of the compounds of the disclosure to effectively treat the patient. Generally, when treating cancer, the dose is sufficient to stop tumor growth or cause tumor regression without producing unacceptable toxicity or side-effects to the patient.

### VII. Intermediates

In some embodiments, the present disclosure provides intermediates useful in the preparation of compounds of Formula (I). Certain intermediates useful in the preparation of a compound of Formula (I) can be found, for example, in the Examples section of the current disclosure.

In some embodiments, an intermediate useful in the preparation of a compound of Formula (I), is an intermediate of Formula (II)

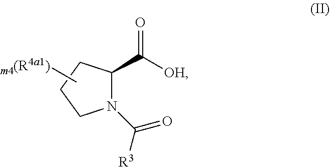

wherein
R³ is C₃₋₆ cycloalkyl substituted with 0 to 5 R^{3b};
each R^{3b} is independently C₁₋₄ alkyl, C₂₋₄ alkenyl, C₂₋₄ alkynyl, halo, C₁₋₄ haloalkyl, cyano, —OH, C₁₋₃ alkoxy, C₁₋₃ haloalkoxy, phenyl, or heteroaryl having 5 to 6 ring members and 1 to 3 heteroatoms each independently N, O or S;
the subscript m4 is an integer from 0 to 2; and
each R^{4a1} is independently C₁₋₄ alkyl, —OH, C₁₋₄ alkyl-OH, C₁₋₄ alkoxy, or halo;
or a pharmaceutically acceptable salt thereof.

In some embodiments, an intermediate useful in the preparation of a compound of Formula (I), is an intermediate of Formula (IIa)

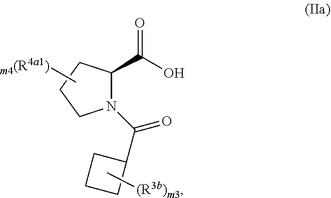

wherein
the subscript m3 is an integer from 0 to 5;
each R^{3b} is independently C₁₋₄ alkyl, C₂₋₄ alkenyl, C₂₋₄ alkynyl, halo, C₁₋₄ haloalkyl, cyano, —OH, C₁₋₃ alkoxy, C₁₋₃ haloalkoxy, phenyl, or heteroaryl having 5 to 6 ring members and 1 to 3 heteroatoms each independently N, O or S;
the subscript m4 is an integer from 0 to 2; and
each R^{4a1} is independently C₁₋₄ alkyl, —OH, C₁₋₄ alkyl-OH, C₁₋₄ alkoxy, or halo;
or a pharmaceutically acceptable salt thereof.

In some embodiments, the intermediate, or the pharmaceutically acceptable salt thereof, is the intermediate of Formula (IIa) wherein the subscript m3 is an integer from 1 to 5. In some embodiments, the intermediate, or the pharmaceutically acceptable salt thereof, is the intermediate of Formula (IIa) wherein the subscript m3 is an integer from 2 to 5. In some embodiments, the intermediate, or the pharmaceutically acceptable salt thereof, is the intermediate of Formula (IIa) wherein the subscript m3 is an integer from 2 to 4. In some embodiments, the intermediate, or the pharmaceutically acceptable salt thereof, is the intermediate of Formula (IIa) wherein the subscript m3 is an integer from 2 to 3. In some embodiments, the intermediate, or the pharmaceutically acceptable salt thereof, is the intermediate of Formula (IIa) wherein the subscript m3 is an integer from 3 to 4. In some embodiments, the intermediate, or the pharmaceutically acceptable salt thereof, is the intermediate of Formula (IIa) wherein the subscript m3 is an integer from 3 to 4. In some embodiments, the intermediate, or the pharmaceutically acceptable salt thereof, is the intermediate of Formula (IIa) wherein the subscript m3 is 3.

In some embodiments, the intermediate, or the pharmaceutically acceptable salt thereof, is the intermediate of Formula (II) or (IIa) wherein each R^{3b} is independently C₁₋₄ alkyl, C₂₋₄ alkenyl, C₂₋₄ alkynyl, halo, C₁₋₄ haloalkyl, or cyano. In some embodiments, the intermediate, or the pharmaceutically acceptable salt thereof, is the intermediate of Formula (II) or (IIa) wherein each R^{3b} is independently C₁₋₄ alkyl, halo, or C₁₋₄ haloalkyl. In some embodiments, the intermediate, or the pharmaceutically acceptable salt thereof, is the intermediate of Formula (II) or (IIa) wherein each R^{3b} is independently halo or C₁₋₄ haloalkyl. In some embodiments, the intermediate, or the pharmaceutically acceptable salt thereof, is the intermediate of Formula (II) or (IIa) wherein each R^{3b} is independently fluoro, or trifluoromethyl.

In some embodiments, the intermediate, or the pharmaceutically acceptable salt thereof, is the intermediate of Formula (II) or (IIa) wherein the subscript m4 is an integer from 1 to 2. In some embodiments, the intermediate, or the pharmaceutically acceptable salt thereof, is the intermediate of Formula (II) or (IIa) wherein the subscript m4 is 0. In some embodiments, the intermediate, or the pharmaceutically acceptable salt thereof, is the intermediate of Formula (II) or (IIa) wherein the subscript m4 is 1. In some embodiments, the intermediate, or the pharmaceutically acceptable salt thereof, is the intermediate of Formula (II) or (IIa) wherein the subscript m4 is 2.

In some embodiments, the intermediate, or the pharmaceutically acceptable salt thereof, is the intermediate of Formula (II) or (IIa) wherein each R^{4a1} is independently C₁₋₄ alkyl, —OH, C₁₋₄ alkyl-OH, C₁₋₄ alkoxy, or halo. In some embodiments, the intermediate, or the pharmaceutically acceptable salt thereof, is the intermediate of Formula (II) or (IIa) wherein each R^{4a1} is independently C₁₋₄ alkyl or halo. In some embodiments, the intermediate, or the pharmaceutically acceptable salt thereof, is the intermediate of Formula (II) or (IIa) wherein each R^{4a1} is independently halo. In some embodiments, the intermediate, or the pharmaceutically acceptable salt thereof, is the intermediate of Formula (II) or (IIa) wherein each R^{4a1} is independently fluoro.

In some embodiments, the intermediate, or the pharmaceutically acceptable salt thereof, is the intermediate of Formula (IIa) wherein
the subscript m3 is an integer from 1 to 5;
each R^{3b} is independently C₁₋₄ alkyl, halo, or C₁₋₄ haloalkyl;
the subscript m4 is an integer from 0 to 2; and
each R^{4a1} is independently C₁₋₄ alkyl or halo.

In some embodiments, the intermediate, or the pharmaceutically acceptable salt thereof, is the intermediate of Formula (IIa) wherein
the subscript m3 is an integer from 1 to 5;
each R^{3b} is independently halo or C₁₋₄ haloalkyl;
the subscript m4 is an integer from 0 to 2; and
each R^{4a1} is independently halo.

In some embodiments, the intermediate, or the pharmaceutically acceptable salt thereof, is the intermediate of Formula (IIa) wherein
the subscript m3 is an integer from 2 to 3;
each R^{3b} is independently halo or C₁₋₄ haloalkyl;
the subscript m4 is an integer from 0 to 2; and
each R^{4a1} is independently halo.

Any of the embodiments described herein for the intermediate of Formula (II) or (IIa) can be combined with any of the embodiments described in this section. For example, any of the embodiments of R³, m3, R^{3b}, m4, R^{4a1} as described herein, can be combined.

In some embodiments, the intermediate is a Building Block described herein. In some embodiments, the intermediate is one of Building Blocks 1-69. In some embodiments, the intermediate is Building Block 4. In some embodiments, the intermediate is Building Block 7. In some embodiments, the intermediate is Building Block 43. In some embodiments, the intermediate is Building Block 47. In some embodiments, the intermediate is Building Block 69.

In some embodiments, the intermediate is a combination of one or more covalently linked Building Blocks.

### VIII. Kits

The present disclosure contemplates kits comprising a compound of Formula (I), (Ia), (Ia1), (Ib), (Ib1), (Ic), or (Ic1) described herein described herein, and pharmaceutical compositions thereof. The kits are generally in the form of a physical structure housing various components, as described below, and can be utilized, for example, in practicing the methods described above.

A kit can include one or more of the compounds disclosed herein (provided in, e.g., a sterile container), which may be in the form of a pharmaceutical composition suitable for administration to a subject. The compounds described herein can be provided in a form that is ready for use (e.g., a tablet, capsule, syringe) or in a form requiring, for example, reconstitution or dilution (e.g., a powder) prior to administration. When the compounds described herein are in a form that needs to be reconstituted or diluted by a user, the kit may also include diluents (e.g., sterile water), buffers, pharmaceutically acceptable excipients, and the like, packaged with or separately from the compounds described herein. Each component of the kit can be enclosed within an individual container, and all of the various containers can be within a single package. A kit of the present disclosure can be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### IX. Examples

The following examples illustrate how various building blocks and exemplary compounds of Formula I are prepared. The following examples are offered to illustrate, but not to limit the claimed disclosure.

### A. Building Blocks

The compounds of Formula I described herein are prepared by covalently linking the building blocks described in this section. The building blocks of the present disclosure are identified in Table 1, below, by Short Hand Name, reagent name, and CAS number, if known. For those without a CAS number, an experimental write-up is provided herein. Uppercase and lowercase lettering in the short hand name is relevant as it can indicate stereochemistry (i.e. 25ClF refers to Fmoc-L-2,5-dichlorophenylalanine while 25Clf refers to Fmoc-D-2,5-dichlorophenylalanine). The order and details related to covalently linking these building blocks are described in another section.

TABLE 1
Building Blocks of the Present Disclosure

| Short Hand | | CAS #/Building |
|---|---|---|
| Name | Reagent Name | Block # |
| 23Pyr5ClF | (S)-2-((((9H-fluoren-9- | Building Block 61 |
| | yl)methoxy)carbonyl)amino)-3-(5-chloro-2- | |
| | (pyridin-3-yl)phenyl)propanoic acid | |
| 25ClF | Fmoc-L-Phe(2,5-DiCl)—OH | 1260614-80-9 |
| 25Clf | Fmoc-D-Phe(2,5-DiCl)—OH | 1260596-66-4 |
| 25FF | (S)-2-((((9H-fluoren-9- | Building Block 18 |
| | yl)methoxy)carbonyl)amino)-3-(2,5- | |
| | difluorophenyl)propanoic acid | |
| 2Aze | Fmoc-L-azetidine-2-carboxylic acid | 136552-06-2 |
| 2aze | Fmoc-D-Azetidine-2-carboxylic acid | 374791-02-3 |
| 2Br5ClF | (R)-2-((((9H-fluoren-9- | Building Block 24 |
| | yl)methoxy)carbonyl)amino)-3-(2-bromo-5- | |
| | chlorophenyl)propanoic acid | |
| 2C15FF | (S)-2-((((9H-fluoren-9- | Building Block 19 |
| | yl)methoxy)carbonyl)amino)-3-(2-chloro-5 | |
| | fluorophenyl)propanoic acid | |
| 2F5ClF | (S)-2-((((9H-fluoren-9- | Building Block 16 |
| | yl)methoxy)carbonyl)amino)-3-(5-chloro-2- | |
| | fluorophenyl)propanoic acid | |
| 2H105ClF | (S)-2-((((9H-fluoren-9- | Building Block 50 |
| | yl)methoxy)carbonyl)amino)-3-(5-chloro-2-(1- | |
| | methyl-1H-pyrazol-4-yl)phenyl)propanoic acid | |
| 2H115ClF | (S)-2-((((9H-fluoren-9- | Building Block 52 |
| | yl)methoxy)carbonyl)amino)-3-(5-chloro-2- | |
| | (thiazol-4-yl)phenyl)propanoic | |
| 2H125ClF | (S)-2-((((9H-fluoren-9- | Building Block 53 |
| | yl)methoxy)carbonyl)amino)-3-(5-chloro-2-(4- | |
| | methylthiazol-5-yl)phenyl)propanoic acid | |
| 2H135ClF | (S)-2-((((9H-fluoren-9- | Building Block 54 |
| | yl)methoxy)carbonyl)amino)-3-(5-chloro-2-(2,4- | |
| | dimethylthiazol-5-yl)phenyl)propanoic acid | |
| 2H145ClF | (S)-2-((((9H-fluoren-9- | Building Block 55 |
| | yl)methoxy)carbonyl)amino)-3-(5-chloro-2- | |
| | (1,3,4-thiadiazol-2-yl)phenyl)propanoic acid | |
| 2H155ClF | (S)-2-((((9H-fluoren-9- | Building Block 56 |
| | yl)methoxy)carbonyl)amino)-3-(5-chloro-2-(2- | |
| | methyl-2H-1,2,3-triazol-4-yl)phenyl)propanoic | |
| | acid | |
| 2H165ClF | (S)-2-((((9H-fluoren-9- | Building Block 57 |
| | yl)methoxy)carbonyl)amino)-3-(5-chloro-2-(2- | |
| | methylthiazol-5-y1)phenyl)propanoic acid | |
| 2H45ClF | (S)-2-((((9H-fluoren-9- | Building Block 48 |
| | yl)methoxy)carbonyl)amino)-3-(5-chloro-2- | |
| | (thiazol-2-yl)phenyl)propanoic acid | |
| 2H55ClF | (S)-2-((((9H-fluoren-9- | Building Block 47 |
| | yl)methoxy)carbonyl)amino)-3-(5-chloro-2- | |
| | (thiazol-5-y1)phenyl)propanoic acid | |
| 2H55FF | (S)-2-((((9H-fluoren-9- | Building Block 58 |
| | yl)methoxy)carbonyl)amino)-3-(5-fluoro-2- | |
| | (thiazol-5-yl)phenyl)propanoic acid | |
| 2H75ClF | (S)-2-((((9H-fluoren-9- | Building Block 49 |
| | yl)methoxy)carbonyl)amino)-3-(5-chloro-2-(1- | |
| | methyl-1H-pyrazol-3-yl)phenyl)propanoic acid | |
| 2H85ClF | (S)-2-((((9H-fluoren-9- | Building Block 63 |
| | yl)methoxy)carbonyl)amino)-3-(5-chloro-2-(1H- | |
| | 1,2,3-triazol-1-yl)phenyl)propanoic acid | |
| 2H95ClF | (S)-2-((((9H-fluoren-9- | Building Block 60 |
| | yl)methoxy)carbonyl)amino)-3-(5-chloro-2-(1- | |
| | methyl-1H-pyrazol-5-y1)phenyl)propanoic acid | |
| 2Me5ClF | (S)-2-((((9H-fluoren-9- | Building Block 20 |
| | yl)methoxy)carbonyl)amino)-3-(5-chloro-2- | |
| | methylphenyl)propanoic acid | |
| 20CF35ClF | (S)-2-((((9H-fluoren-9- | Building Block 21 |
| | yl)methoxy)carbonyl)amino)-3-(5-chloro-2- | |
| | (trifluoromethoxy)phenyl)propanoic acid | |
| 20EtCF35ClF | (S)-2-((((9H-fluoren-9- | Building Block 33 |
| | yl)methoxy)carbonyl)amino)-3-(5-chloro-2- | |
| | (2,2,2-trifluoroethoxy)phenyl)propanoic acid | |
| 20Me5ClF | Fmoc-L-Phe(20Me,5Cl)-OH | Building Block 22 |
| 2Ph5ClF | (S)-2-((((9H-fluoren-9- | Building Block 62 |
| | yl)methoxy)carbonyl)amino)-3-(4-chloro-[1,1′- | |
| | biphenyl]-2-yl)propanoic acid | |
| 3CIF | Fmoc-L-3Cl-Phenylalanine | 198560-44-0 |
| 3CNF | Fmoc-L-Phe(3-CN)—OH | 205526-36-9 |
| 3FF | Fmoc-L-Phe(3-F)—OH | 198560-68-8 |
| 44FP | Fmoc-L-44-difluoroproline | 203866-21-1 |
| 5Br2ClF | Fmoc-L-Phe(5Br,2Cl)—OH | Building Block 23 |
| 5Cl2IF | Fmoc-L-Phe(5Cl,2I)—OH | Building Block 25 |
| 5Cl2OcHexF | Fmoc-L-Phe(5Cl,2OcHex)-OH | Building Block 32 |
| 5Cl2OcPenF | Fmoc-L-Phe(5Cl,2OcPen)-OH | Building Block 31 |
| 5Cl2OcPrF | Fmoc-L-Phe(5Cl,2OcPr)—OH | Building Block 28 |
| 5Cl2OMePenF | (S)-2-((((9H-fluoren-9- | Building Block 30 |
| | yl)methoxy)carbonyl)amino)-3-(5-chloro-2- | |
| | (cyclopentylmethoxy)phenyl)propanoic acid | |
| 5Cl2OPhF | (S)-2-((((9H-fluoren-9- | Building Block 29 |
| | yl)methoxy)carbonyl)amino)-3-(5-chloro-2- | |
| | phenoxyphenyl)propanoic acid | |
| a | Fmoc-D-Alanine | 79990-15-1 |
| A | Fmoc-L-Alanine | 35661-39-3 |
| AA0011 | Fmoc-trans-4-fluoro-Pro-OH | 203866-20-0 |
| abu | Fmoc-D-2-aminobutanoic acid | 170642-27-0 |
| Abu | Fmoc-L-2-aminobutanoic acid | 135112-27-5 |
| Acah | Acetic Anhydride | 108-24-7 |
| Acd0317 | 1-(Trifluoromethyl)cyclopropane-1-carboxylic | 277756-46-4 |
| | acid | |
| Acd0347 | oxazole-4-carboxylic acid | 23012-13-7 |
| Acd0401 | 1-Ethynylcyclopropanecarboxylic acid | 933755-97-6 |
| Acd0423 | 2,2-Dimethylbutyric acid | 595-37-9 |
| Acd0436 | 3,3,3-Trifluoro-2,2-dimethylpropionic acid | 889940-13-0 |
| Acd0438 | 2,2-dimethylbut-3-ynoic acid | 56663-76-4 |
| Acd0445 | 1-methyl-1H-imidazole-4-carboxylic acid | 41716-18-1 |
| Acd0486 | (R)-3,3,3-trifluoro-2-hydroxy-2-methylpropionic | 44864-47-3 |
| | acid | |
| Acd0487 | (S)-3,3,3-trifluoro-2-hydroxy-2-methylpropionic | 24435-45-8 |
| | acid | |
| Acd0498 | 2-ethyl-2-methylbutanoic acid | 19889-37-3 |
| Acd0503 | trifluoromethyl cyclopentane carboxylic acid | 277756-44-2 |
| Acd0504 | trifluoromethyl cyclohexane carboxylic acid | 180918-40-5 |
| Acd0505 | trifluoromethyl cyclobutane carboxylic acid | 277756-45-3 |
| Acd0520 | 1-ethylcyclopentane-1-carboxylic acid, 17206-19-8 | 17206-19-8 |
| Acd0525 | 2-(trifluoromethyl)bicyclo[2.2.1]heptane-2- | 1896752-94-5 |
| | carboxylic acid | |
| Acd0532 | 2-(trifluoromethyl)oxane-2-carboxylic acid | 2229402-19-9 |
| Acd0533 | 4-(trifluoromethyl)oxane-4-carboxylic acid | 1524761-14-5 |
| Acd0536 | (1-(trifluoromethyl)cyclohexane-1-carbonyl)-L- | Building Block 2 |
| | proline | |
| Acd0540 | 2-(trifluoromethyl)spiro[3.3]heptane-2-carboxylic | 2166650-85-5 |
| | acid | |
| Acd0542 | 4-(1,1-difluoroethyl)tetrahydro-2H-pyran-4- | 1781078-83-8 |
| | carboxylic acid | |
| Acd0544 | 2-cyclopropyl-2-hydroxypropanoic acid | 99848-37-0 |
| Acd0559 | 3,3,3-trifluoro-2-methoxy-2-methylpropanoic acid | 56135-02-5 |
| | (racemic) | |
| Acd0573 | 1-ethynylcyclopentane-1-carboxylic acid | 887590-70-7 |
| Acd0574 | (S)-4,4-difluoro-1-(1- | Building Block 1 |
| | (trifluoromethyl)cyclohexane-1- | |
| | carbonyl)pyrrolidine-2-carboxylic acid | |
| Acd0575 | (2S,4R)-4-fluoro-1-(1- | Building Block 4 |
| | (trifluoromethyl)cyclohexane-1- | |
| | carbonyl)pyrrolidine-2-carboxylic acid | |
| Acd0577 | 2-cyclopropyl-2-methoxypropanoic acid | 1247669-87-9 |
| | (racemic) | |
| Acd0578 | 2-hydroxy-3-methyl-2-(trifluoromethyl)butanoic | 1823842-47-2 |
| | acid (racemic) | |
| Acd0588 | 3,3-difluoro-1-(trifluoromethyl)cyclobutane-1- | 2167095-52-3 |
| | carboxylic acid | |
| Acd0592 | 4-methyltetrahydro-2H-thiopyran-4-carboxylic | 1713163-23-5 |
| | acid 1,1-dioxide | |
| Acd0594 | 3-(trifluoromethyl)bicyclo[3.1.0]hexane-3- | 1558160-95-4 |
| | carboxylic acid | |
| Acd0596 | (R)-2-(difluoromethoxy)-3,3,3-trifluoro-2- | Building Block 14 |
| | methylpropanoic acid | |
| Acd0625 | (R)-2-((tert-butoxycarbonyl)amino)-3,3,3- | Building Block 12 |
| | trifluoropropanoic acid | |
| Acd0626 | (S)-2-((tert-butoxycarbonyl)amino)-3,3,3- | Building Block 13 |
| | trifluoropropanoic acid | |
| Acd0687 | (R)-2-hydroxy-2-phenylpropanoic acid | 3966-30-1 |
| Acd0688 | 3,3,3-trifluoro-2-hydroxy-2-phenylpropanoic acid | 55519-22-7 |
| Acd0703 | (2S,4R)-1-(4,4-difluoro-1- | Building Block 10 |
| | (trifluoromethyl)cyclohexane-1-carbonyl)-4- | |
| | fluoropyrrolidine-2-carboxylic acid | |
| Acd0733 | (2S,4R)-1-(6,6-difluoro-2- | Building Block 8 |
| | (trifluoromethyl)spiro[3.3]heptane-2-carbonyl)-4- | |
| | fluoropyrrolidine-2-carboxylic acid | |
| Acd0734 | (2S,4R)-1-(3,3-difluoro-1- | Building Block 9 |
| | (trifluoromethyl)cyclopentane-1-carbonyl)-4- | |
| | fluoropyrrolidine-2-carboxylic acid | |
| Acd0737 | 2-cyclopropyl-3,3,3-trifluoro-2-hydroxypropanoic | 1893991-91-7 |
| | acid | |
| Acd0741 | (2S,4R)-1-(1-(difluoromethyl)-3,3- | Building Block 11 |
| | difluorocyclobutane-1-carbonyl)-4- | |
| | fluoropyrrolidine-2-carboxylic acid | |
| Acd0747 | 3-fluoro-1-(trifluoromethyl)cyclobutane-1- | 2168389-13-5 |
| | carboxylic acid (diasteromeric mixture) | |
| Acd0780 | (R)-3,3,3-trifluoro-2-methoxy-2-methylpropanoic | 166584-04-9 |
| | acid | |
| Acd0794 | (2S,4R)-4-fluoro-1-(2- | Building Block 6 |
| | (trifluoromethyl)tetrahydro-2H-pyran-2- | |
| | carbonyl)pyrrolidine-2-carboxylic acid | |
| Acd0799 | (2S,4R)-4-fluoro-1-(4- | Building Block 70 |
| | (trifluoromethyl)tetrahydro-2H-pyran-4- | |
| | carbonyl)pyrrolidine-2-carboxylic acid | |
| Acd0801 | (2S,4R)-4-fluoro-1-(2- | Building Block 3 |
| | (trifluoromethyl)bicyclo[2.2.1]heptane-2- | |
| | carbonyl)pyrrolidine-2-carboxylic acid | |
| Acd0805 | (1s,3s)-3-hydroxy-1- | 2416234-23-4 |
| | (trifluoromethyl)cyclobutane-1-carboxylic acid | |
| Acd0807 | (1R,3R)-3-methoxy-1- | 2624108-66-1 |
| | (trifluoromethyl)cyclopentane-1-carboxylic acid | |
| | (racemic) | |
| Acd0809 | 1-ethynylcyclobutane-1-carboxylic acid | 887590-67-2 |
| Acd0810 | 1-cyanocyclobutane-1-carboxylic acid | 30491-91-9 |
| Acd0811 | 2-cyanospiro[3.3]heptane-2-carboxylic acid | 1487965-23-0 |
| Acd0813 | 3-(1,1-difluoroethyl)oxetane-3-carboxylic acid | 178090955-8 |
| ACF3 | (S)-2-((((9H-fluoren-9- | 181128-48-3 |
| | yl)methoxy)carbonyl)amino)-4,4,4- | |
| | trifluorobutanoic acid | |
| Acpc | Fmoc-1-aminocyclopropane-1-carboxylic acid | 126705-22-4 |
| Aib | Fmoc-a-aminoisobutyric acid | 94744-50-0 |
| Alc0004 | 2-Cyclohexylethanol | 4442-79-9 |
| Alc0045 | Butyl Alcohol | 71-36-3 |
| Alc0046 | 1-Propanol | 71-23-8 |
| Alc0050 | Isobutanol | 78-83-1 |
| Alc0070 | cyclopropanemethanol | 2516-33-8 |
| Ald0003 | Cyclohexaneacetaldehyde | 5664-21-1 |
| aMeabu | Fmoc-D-isovaline | 1231709-22-0 |
| Aze | Fmoc-L-azetidine-3-carboxylic acid | 193693-64-0 |
| Cba | Fmoc-D-cyclobuylalanine | 478183-63-0 |
| CBF | (2S)-3-(3,3-difluorocyclobutyl)-2-{[(9H-fluoren- | Building Block 27 |
| | 9-ylmethoxy)carbonyl]amino}propanoic acid | |
| CBG | (2S)-2-cyclobutyl-2-({[(9H-fluoren-9- | 1391630-31-1 |
| | yl)methoxy]carbonylamino)acetic acid | |
| CD₃OD | Deuterated Methanol | 811-98-3 |
| CFFB | Fmoc-3,3-difluoro-cyclobutanecarboxylic acid | 1936532-04-5 |
| cFp | (2R,4R)-1-{[(9H-fluoren-9- | 1932387-77-3 |
| | yl)methoxy]carbonyl}-4-fluoropyrrolidine-2- | |
| | carboxylic acid | |
| CPA | Fmoc-β-Cyclopropyl-L-Alanine | 214750-76-2 |
| cPrg | Fmoc-D-cyclopropyl glycine | 923012-40-2 |
| cPrG | Fmoc-L-cyclopropyl glycine | 1212257-18-5 |
| CVa | Fmoc-L-cyclovaline | 885951-77-9 |
| CycBuA | Fmoc-L-Ala(β-cyclobutyl)-OH | 478183-62-9 |
| DabDde | Fmoc-L-Dab(Dde)-OH | 235788-61-1 |
| EtOH | Ethanol | 64-17-5 |
| F | Fmoc-L-Phenylalanine | 35661-40-6 |
| G | Fmoc-Glycine | 29022-11-5 |
| Gaba | Fmoc-4-aminobutyric acid | 116821-47-7 |
| hKBoc | Fmoc-L-hLys(Boc)-OH | 194718-17-7 |
| hL | Fmoc-L-homoleucine | 180414-94-2 |
| hSerTrt | Fmoc-L-homoSer(Trt)-OH | 111061-55-3 |
| hSerMe | Fmoc-L-hSer(OMe)-OH | 173212-86-7 |
| IsoBu | Isobutyryl chloride | 79-30-1 |
| KBoc | Fmoc-L-Lys(Boc)-OH | 71989-26-9 |
| kBoc | Fmoc-D-Lys(Boc)-OH | 92122-45-7 |
| Kac | Fmoc-L-Lys(Ac)-OH | 159766-56-0 |
| KiPr | Fmoc-L-Lysine(iPr,Boc)-OH | 201003-48-7 |
| KMe | Fmoc-L-Lys(Boc)(Me)-OH | 951695-85-5 |
| KMe2 | Fmoc-L-Lys(Me)2-OH•HCl | 252049-10-8 |
| KMor | (S)-2-((((9H-fluoren-9- | 2349553-17-7 |
| | yl)methoxy)carbonyl)amino)-6- | |
| | morpholinohexanoic acid | |
| KMtt | Fmoc-L-Lys(Mtt)-OH | 167393-62-6 |
| KTfa | Fmoc-L-Lys(Tfa)-OH | 76265-69-5 |
| KTFE | Fmoc-L-Lys(trifluoroethane,Boc)-OH | Building Block 15 |
| L | Fmoc-L-Leucine | 35661-60-0 |
| L | Fmoc-D-Leucine | 114360-54-2 |
| LysO | (S)-Fmoc-2-amino-6-tert-butoxy-hexanoic acid | 1354752-71-8 |
| MeOH | Methanol | 67-56-1 |
| B2BE | Bis(2-bromoethyl) ether | 5414-19-7 |
| Mor0003 | (S)-2-((((9H-fluoren-9- | Building Block 67 |
| | yl)methoxy)carbonyl)amino)-6-(4,4- | |
| | difluoropiperidin-1-yl)hexanoic acid | |
| Nle | Fmoc-L-Norleucine | 77284-32-3 |
| NMeK | Fmoc-N—Me-L-Lys(Boc)-OH | 197632-76-1 |
| NMeKMe | Fmoc-NMe-L-Lys(Boc)(Me)—OH | Building Block 68 |
| Nva | Fmoc-D-Norvaline | 144701-24-6 |
| Nva | Fmoc-L-2-aminovaleric acid | 135112-28-6 |
| Omor | Fmoc-L-Orn(Morpholine)-OH | 2350138-22-4 |
| P | Fmoc-D-Proline | 101555-62-8 |
| P | Fmoc-L-Proline | 71989-31-6 |
| Phe0008 | (S)-2-((((9H-fluoren-9- | Building Block 17 |
| | yl)methoxy)carbonyl)amino)-3-(3,6-dichloro-2- | |
| | fluorophenyl)propanoic acid | |
| Phe0013 | (S)-2-((((9H-fluoren-9- | Building Block 26 |
| | yl)methoxy)carbonyl)amino)-3-(5-chloro-2- | |
| | (difluoromethoxy)phenyl)propanoic acid | |
| Phe0023 | (S)-2-((((9H-fluoren-9- | Building Block 34 |
| | yl)methoxy)carbonyl)amino)-3-(5-chloro-2- | |
| | (cyclobutylmethoxy)phenyl)propanoic acid | |
| Phe0024 | (S)-2-((((9H-fluoren-9- | Building Block 35 |
| | yl)methoxy)carbonyl)amino)-3-(5-chloro-2- | |
| | cyclobutoxyphenyl)propanoic acid | |
| Phe0034 | (S)-2-((((9H-fluoren-9- | Building Block 36 |
| | yl)methoxy)carbonyl)amino)-3-(5-chloro-2- | |
| | cyclopropoxyphenyl)propanoic acid | |
| Phe0042 | (S)-2-((((9H-fluoren-9- | Building Block 37 |
| | yl)methoxy)carbonyl)amino)-3-(5-chloro-2- | |
| | (cyclopropylmethoxy)pyridin-3-yl)propanoic acid | |
| Phe0046 | (S)-2-((((9H-fluoren-9- | Building Block 39 |
| | yl)methoxy)carbonyl)amino)-3-(5-chloro-2-(1,3- | |
| | dimethyl-1H-pyrazol-4-yl)phenyl)propanoic acid | |
| Phe0047 | (S)-2-((((9H-fluoren-9- | Building Block 40 |
| | yl)methoxy)carbonyl)amino)-3-(5-chloro-2-(5- | |
| | fluoropyridin-3-y1)phenyl)propanoic acid | |
| Phe0048 | (S)-2-((((9H-fluoren-9- | Building Block 41 |
| | yl)methoxy)carbonyl)amino)-3-(5-chloro-2-(1- | |
| | (difluoromethyl)-1H-pyrazol-4- | |
| | yl)phenyl)propanoic acid | |
| Phe0049 | (S)-2-((((9H-fluoren-9- | Building Block 42 |
| | yl)methoxy)carbonyl)amino)-3-(5-chloro-2-(1- | |
| | (trifluoromethyl)-1H-pyrazol-4- | |
| | yl)phenyl)propanoic acid | |
| Phe0050 | (S)-2-((((9H-fluoren-9- | Building Block 38 |
| | yl)methoxy)carbonyl)amino)-3-(5-chloro-2- | |
| | (pyridin-2-yl)phenyl)propanoic acid | |
| Phe0051 | (S)-2-((((9H-fluoren-9- | Building Block 64 |
| | yl)methoxy)carbonyl)amino)-3-(5-chloro-2- | |
| | (dimethylamino)phenyl)propanoic acid | |
| Phe0053 | (S)-2-((((9H-fluoren-9- | Building Block 65 |
| | yl)methoxy)carbonyl)amino)-3-(5-chloro-2- | |
| | (methoxymethyl)phenyl)propanoic acid | |
| Phe0055 | (S)-2-((((9H-fluoren-9- | Building Block 43 |
| | yl)methoxy)carbonyl)amino)-3-(5-chloro-2-(1,5- | |
| | dimethyl-1H-pyrazol-4-yl)phenyl)propanoic acid | |
| Phe0056 | (S)-2-((((9H-fluoren-9- | Building Block 44 |
| | yl)methoxy)carbonyl)amino)-3-(5-chloro-2-(1,3- | |
| | dimethyl-1H-pyrazol-5-yl)phenyl)propanoic acid | |
| Phe0057 | (S)-2-((((9H-fluoren-9- | Building Block 45 |
| | yl)methoxy)carbonyl)amino)-3-(5-chloro-2- | |
| | (pyrimidin-5-yl)phenyl)propanoic acid | |
| Phe0058 | (S)-2-((((9H-fluoren-9- | Building Block 46 |
| | yl)methoxy)carbonyl)amino)-3-(5-chloro-2- | |
| | (morpholinomethyl)phenyl)propanoic acid | |
| Phe0060 | (S)-2-((((9H-fluoren-9- | Building Block 59 |
| | yl)methoxy)carbonyl)amino)-3-(5-chloro-2-(5- | |
| | methyl-1,3,4-thiadiazol-2-yl)phenyl)propanoic | |
| Phe0062 | (S)-2-((((9H-fluoren-9- | Building Block 51 |
| | yl)methoxy)carbonyl)amino)-3-(5-fluoro-2-(1- | |
| | methyl-1H-pyrazol-4-yl)phenyl)propanoic acid | |
| Pip | N-Fmoc-D-pipecolic acid | 101555-63-9 |
| Pip0002 | (S)-2-((((9H-fluoren-9- | Building Block 66 |
| | yl)methoxy)carbonyl)amino)-6-oxo-6-(piperidin- | |
| | 1-yl)hexanoic acid | |
| RA211 | 2-Thiazolecarboxylic acid | 14190-59-1 |
| RA230 | 6-(methylamino)picolinic acid | 1250806-91-7 |
| RA245 | 4-Carboxythiazole | 3973-08-8 |
| Sar | Fmoc-Sarcosine | 77128-70-2 |
| SHOp | Fmoc-(2R,4S)-4-hydroxypyrrolidine-2-carboxylic | 268729-12-0 |
| | acid (tBu) | |
| SHOP | Fmoc-(2S,4S)-4-hydroxypyrrolidine-2-carboxylic | 189249-10-3 |
| | acid | |
| sMe | Fmoc-D-Ser(Me)-OH | 1569103-64-5 |
| T | Fmoc-L-Threonine | 73731-37-0 |
| TBA | Fmoc-L-t-butyl-Alanine | 139551-74-9 |
| tFp | (2R,4S)-1-{[(9H-fluoren-9-yl)methoxy]carbonyl}-4- | 913820-87-8 |
| | fluoropyrrolidine-2-carboxylic acid | |
| Tic | Fmoc-(3S-)-1,2,3,4-tetrahydroisoquinoline-3- | 136030-33-6 |
| | carboxylic acid | |
| Tic0004 | 2-(((9H-fluoren-9-yl)methoxy)carbonyl)-6- | 1344158-46-8 |
| | hydroxy-1,2,3,4-tetrahydroisoquinoline-1- | |
| | carboxylic acid | |
| Tic0005 | 2-(((9H-fluoren-9- | 204320-59-2 |
| | yl)methoxy)carbonyl)isoindoline-1-carboxylic | |
| | acid | |
| TicOH | (S)-2-(((9H-fluoren-9-yl)methoxy)carbonyl)-7- | 178432-49-0 |
| | hydroxy-1,2,3,4-tetrahydroisoquinoline-3- | |
| | carboxylic acid | |
| Tle | Fmoc-L-Tle-OH | 132684-60-7 |
| V | Fmoc-L-Valine | 68858-20-8 |
| B0001 | 1-(4-(4,4,5,5-tetramethyl-1,3,2-dioxaborolan-2- | 1227068-67-8 |
| | y1)-3,6-dihydropyridin-1(2H)-yl)ethan-1-one | |
| B0002 | 2-(2,5-dimethylthiophen-3-yl)-4,4,5,5- | 942070-20-4 |
| | tetramethyl-1,3,2-dioxaborolane | |
| B0003 | 4-(4,4,5,5-tetramethyl-1,3,2-dioxaborolan-2- | 1310384-24-7 |
| | yl)cyclohex-3-en-1-ol | |
| B0004 | 2-(3,6-dihydro-2H-pyran-4-yl)-4,4,5,5- | 287944-16-5 |
| | tetramethyl-1,3,2-dioxaborolane | |
| B0005 | 1-(oxetan-3-yl)-4-(4,4,5,5-tetramethyl-1,3,2- | 1339890-99-1 |
| | dioxaborolan-2-y1)-1H-pyrazole | |
| B0006 | 2-Methoxy-5-(4,4,5,5-tetramethyl-1,3,2- | 445264-61-9 |
| | dioxaborolan-2-yl)pyridine | |
| B0007 | 1,3-dimethyl-4-(4,4,5,5-tetramethyl-1,3,2- | 1046832-21-6 |
| | dioxaborolan-2-yl)-1H-pyrazole | |
| Dip0018 | (2S,4R)-1-(3,3-difluoro-1- | Building Block 69 |
| | (trifluoromethyl)cyclobutane-1-carbonyl)-4- | |
| | fluoropyrrolidine-2-carboxylic acid | |
| Dip0019 | (2S,4R)-4-fluoro-1-((R)-3,3,3-trifluoro-2-(2-(2- | Building Block 7 |
| | methoxyethoxy)ethoxy)-2- | |
| | methylpropanoyl)pyrrolidine-2-carboxylic acid | |
| Dip0023 | (2S,4R)-4-fluoro-1-(1- | Building Block 5 |
| | (trifluoromethyl)cyclopropane-1- | |
| | carbonyl)pyrrolidine-2-carboxylic acid | |
| AcCl | Acetyl chloride | 75-36-5 |

### Building Block 1: Preparation of (S)-4,4-difluoro-1-(1-(trifluoromethyl)cyclohexane-1-carbonyl)pyrrolidine-2-carboxylic Acid

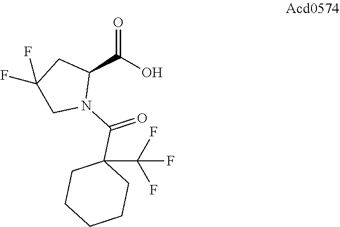 [SMILES: O=C(O)[C@@H]1CC(F)(F)CN1C(=O)C1(C(F)(F)F)CCCCC1]

1-(trifluoromethyl)cyclohexane-1-carboxylic acid (500 mg, 2.55 mmol) was dissolved in thionyl chloride (3.6 ml, 51 mmol) and was heated at reflux for 3 h. The mixture was allowed to cool and thionyl chloride was removed via azeotrope with toluene. The crude was taken onto the next step without further purification.

Methyl (S)-4,4-difluoropyrrolidine-2-carboxylate was dissolved in 5 ml of DCM. Pyridine (615 ul, 7.65 mmol) was added and the mixture was cooled to 0° C. Dropwise, a dissolved solution of 1-(trifluoromethyl)cyclohexane-1-carbonyl chloride was added to the reaction. The reaction was warmed to room temperature and allowed to run for 12 h. The reaction was quenched with NaHCO3 and the mixture was extracted 3× with DCM. The combined extracts was dried over MgSO4, filtered, and concentrated to provide methyl (S)-4,4-difluoro-1-(1-(trifluoromethyl)cyclohexane-1-carbonyl)pyrrolidine-2-carboxylate (743 mg, 85%), ESI MS m/z 343.1

Methyl (S)-4,4-difluoro-1-(1-(trifluoromethyl)cyclohexane-1-carbonyl)pyrrolidine-2-carboxylate (500 mg, 1.36 mmol) was dissolved in 10 ml of dioxanes. A dissolved solution of LiOH (112 mg, 2.73 mmol) in 5 ml of water was added to this reaction and allowed to run for 2 h. The reaction was quenched with 1N HCl, and extracted 3× with EtOAc. Combined extracts was dried over MgSO4, filtered and concentrated. The crude product was purified by column chromatography (80% EtOAC/hexanes) to afford (S)-4,4-difluoro-1-(1-(trifluoromethyl)cyclohexane-1-carbonyl)pyrrolidine-2-carboxylic acid (450 mg. 93%) as a white powder, ESI MS m/z 329.1

### Building Block 2: Preparation of (1-(trifluoromethyl)cyclohexane-1-carbonyl)-L-proline

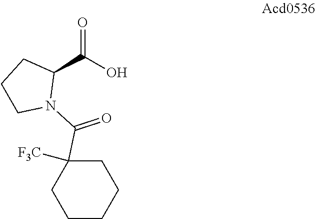 [SMILES: O=C(O)[C@@H]1CCCN1C(=O)C1(C(F)(F)F)CCCCC1]

This compound was prepared following the general synthetic sequence described for the preparation of Building Block 1 using methyl L-proline instead of methyl (S)-4,4-difluoropyrrolidine-2-carboxylate. ESI MS m/z 293.12

### Building Block 3: Preparation of (2S,4R)-4-fluoro-1-(2-(trifluoromethyl)bicyclo[2.2.1]heptane-2-carbonyl)pyrrolidine-2-carboxylic Acid

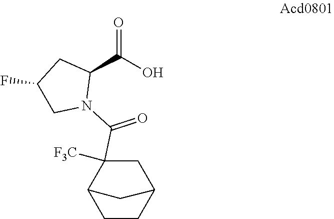 [SMILES: O=C(O)[C@@H]1C[C@@H](F)CN1C(=O)C1(C(F)(F)F)CC2CCC1C2]

This compound was prepared following the general synthetic sequence described for the preparation of Building Block 1 using 2-(trifluoromethyl)bicyclo[2.2.1]heptane-2-carboxylic acid and methyl (2S,4R)-4-fluoropyrrolidine-2-carboxylate. ESI MS m/z 323.12

### Building Block 4: Preparation of (2S,4R)-4-fluoro-1-(1-(trifluoromethyl)cyclohexane-1-carbonyl)pyrrolidine-2-carboxylic Acid

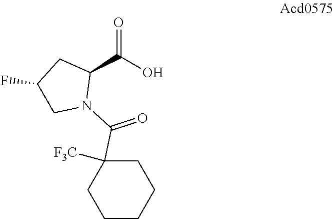 [SMILES: O=C(O)[C@@H]1C[C@@H](F)CN1C(=O)C1(C(F)(F)F)CCCCC1]

This compound was prepared following the general synthetic sequence described for the preparation of Building Block 1 using 1-(trifluoromethyl)cyclohexane-1-carboxylic acid and methyl (2S,4R)-4-fluoropyrrolidine-2-carboxylate. ESI MS m/z 311.10

### Building Block 5: Preparation of (2S,4R)-4-fluoro-1-(1-(trifluoromethyl)cyclopropane-1-carbonyl)pyrrolidine-2-carboxylic Acid

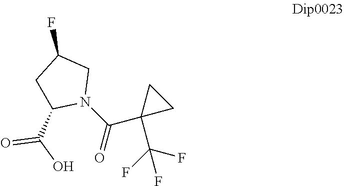 [SMILES: O=C(O)[C@@H]1C[C@@H](F)CN1C(=O)C1(C(F)(F)F)CC1]

This compound was prepared following the general synthetic sequence described for the preparation of Building Block 1 using 1-(trifluoromethyl)cyclopropane-1-carboxylic acid and methyl (2S,4R)-4-fluoropyrrolidine-2-carboxylate ESI MS m/z 269.07

### Building Block 6: Preparation of (2S,4R)-4-fluoro-1-[2-(trifluoromethyl)oxane-2-carbonyl]pyrrolidine-2-carboxylic Acid

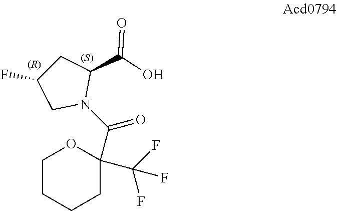

A mixture of methyl (2S,4R)-4-fluoropyrrolidine-2-carboxylate (3.6 g, 19.572 mmol, 1 equiv, 80%), 2-(trifluoromethyl)oxane-2-carboxylic acid (3.88 g, 19.572 mmol, 1.00 equiv) TCFH (8.22 g, 29.35 mmol, 1.5 equiv) and NMI (8.03 g, 97.860 mmol, 5 equiv) in ACN (50 mL) was stirred for 16 h at 25° C. under nitrogen atmosphere. The mixture together with EB2128270-100 was purified directly by reverse flash chromatography. This resulted in methyl (2S,4R)-4-fluoro-1-[2-(trifluoromethyl)oxane-2-carbonyl]pyrrolidine-2-carboxylate (3.5 g, 54.64%) as a white solid. LCMS: (ESI, m/z): [M+H]⁺=328.

The mixture of methyl (2S,4R)-4-fluoro-1-[2-(trifluoromethyl)oxane-2-carbonyl]pyrrolidine-2-carboxylate (4.5 g, 13.750 mmol, 1 equiv) and NaOH (2.75 g, 68.750 mmol, 5 equiv) in MeOH (50 mL)/water (50 mL) was stirred for 16 h at 20° C. The methanol was evaporated in vacuo. The water phase was acidified by the addition of HCl (1N) and extracted with ethyl acetate (200 mL×2). The organic layer was dried over anhydrous Na₂SO₄ and concentrated in vacuo. This resulted in (2S,4R)-4-fluoro-1-[2-(trifluoromethyl)oxane-2-carbonyl]pyrrolidine-2-carboxylic acid (4.0020 g, 92.03%) as a light yellow solid. LCMS: (ESI, m/z): [M+H]⁺=314.0.

### Building Block 7: Preparation of (2S,4R)-4-fluoro-1-((R)-3,3,3-trifluoro-2-(2-(2-methoxyethoxy)ethoxy)-2-methylpropanoyl)pyrrolidine-2-carboxylic Acid

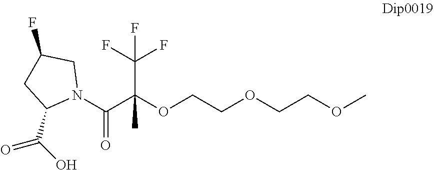 [SMILES: COCCOCCO[C@](C)(C(=O)N1C[C@H](F)C[C@H]1C(=O)O)C(F)(F)F]

To a solution of (R)-3,3,3-trifluoro-2-hydroxy-2-methylpropanoic acid (6.7 g, 42.38 mmol, 1 eq) in DMF (200 mL) was added K₂CO₃ (11.72 g, 84.77 mmol, 2 eq) and bromomethylbenzene (8.70 g, 50.86 mmol, 6.04 mL, 1.2 eq). The mixture was stirred at 20° C. for 1 hr. TLC (Petroleum ether:Ethyl acetate=5:1) indicated (R)-3,3,3-trifluoro-2-hydroxy-2-methylpropanoic acid was consumed completely and one new spot formed. The reaction mixture was poured into 100 mL ammonia chloride, and then extracted with ethyl acetate 200 mL (100 mL×2). The combined organic layers were dried over anhydrous sodium sulfate, filtered and the filtrate was concentrated under reduced pressure to give a residue. The residue was purified by column chromatography (SiO₂, Petroleum ether:Ethyl acetate=200:1 to 5:1) to give benzyl (R)-3,3,3-trifluoro-2-hydroxy-2-methylpropanoate (7.2 g, crude) as a white oil.

To a solution of benzyl (R)-3,3,3-trifluoro-2-hydroxy-2-methylpropanoate (3.6 g, 14.50 mmol, 1 eq) and 1-(2-bromoethoxy)-2-methoxy-ethane (5.31 g, 29.01 mmol, 2 eq) in DMF (150 mL) was added NaH (638.14 mg, 15.96 mmol, 60% purity, 1.1 eq) at 0° C. The mixture was stirred at 20° C. for 12 hr. TLC (Petroleum ether:Ethyl acetate=5:1) indicated reaction completion. The reaction mixture was poured into 100 mL ammonia chloride solution, and then extracted with ethyl acetate 180 mL (90 mL×2). The combined organic layers were dried over anhydrous sodium sulfate, filtered and the filtrate was concentrated under reduced pressure to give a residue. The residue was purified by column chromatography (SiO₂, Petroleum ether:Ethyl acetate=200:1 to 5:1) to give benzyl (R)-3,3,3-trifluoro-2-(2-(2-methoxyethoxy)ethoxy)-2-methylpropanoate (6.7 g, crude) as a yellow oil.

A mixture of benzyl (R)-3,3,3-trifluoro-2-(2-(2-methoxyethoxy)ethoxy)-2-methylpropanoate (4.3 g, 12.27 mmol, 1 eq) in MeOH (150 mL) was added Pd/C (3 g, 10% purity) at 20° C. and then the mixture was degassed and purged with H₂ 3 times, and then the mixture was stirred at 50° C. for 2 h under H₂ atmosphere (15 psi). TLC (petroleum ether:ethyl acetate=1:1) indicated starting material was consumed completely. The reaction was filtered, the filtrate was concentrated under reduced pressure to give (R)-3,3,3-trifluoro-2-(2-(2-methoxyethoxy)ethoxy)-2-methylpropanoic acid (6.2 g, crude) as a yellow oil.

To a solution of (R)-3,3,3-trifluoro-2-(2-(2-methoxyethoxy)ethoxy)-2-methylpropanoic acid (5.6 g, 21.52 mmol, 1 eq) in DCM (90 mL) was added oxalyl dichloride (8.19 g, 64.56 mmol, 5.65 mL, 3 eq) and DMF (157.31 mg, 2.15 mmol, 165.59 μL, 0.1 eq) at 0° C. The mixture was stirred at 0° C. for 1 h. The reaction mixture was concentrated under reduced pressure to give (R)-3,3,3-trifluoro-2-(2-(2-methoxyethoxy)ethoxy)-2-methylpropanoyl chloride (6 g, crude) as a colorless oil.

To a solution of methyl (2S,4R)-4-fluoropyrrolidine-2-carboxylate (3.76 g, 20.48 mmol, 1 eq, HCl) in DCM (50 mL) was added TEA (6.22 g, 61.44 mmol, 8.55 mL, 3 eq) at 0° C. Then a solution of (R)-3,3,3-trifluoro-2-(2-(2-methoxyethoxy)ethoxy)-2-methylpropanoyl chloride (5.99 g, 21.50 mmol, 1.05 eq) in DCM (50 mL) was added into the above mixture at 0° C. The mixture was stirred at 20° C. for 12 h. LCMS showed the reaction was completed and desired mass was detected. The reaction mixture was concentrated under reduced pressure to give a residue. The residue was purified by column chromatography (SiO₂, Petroleum ether:Ethyl acetate=10:1 to 0:1) to give methyl (2S,4R)-4-fluoro-1-((R)-3,3,3-trifluoro-2-(2-(2-methoxyethoxy)ethoxy)-2-methylpropanoyl)pyrrolidine-2-carboxylate (7.3 g, 18.75 mmol, 91.56% yield) as a yellow oil.

To a solution of methyl (2S,4R)-4-fluoro-1-((R)-3,3,3-trifluoro-2-(2-(2-methoxyethoxy)ethoxy)-2-methylpropanoyl)pyrrolidine-2-carboxylate (7.74 g, 19.88 mmol, 1 eq) in THF (60 mL) and MeOH (60 mL) was added LiOH·H₂O (1.67 g, 39.76 mmol, 2 eq) at 0° C. The mixture was stirred at 20° C. for 12 h. LCMS showed Compound 6 was consumed completely and desired mass was detected. The reaction mixture was adjust pH˜5 by saturated solution of citric acid, some solid separated, then filtered, the filtrate was concentrated under reduced pressure to give a residue. The residue was purified by column chromatography (SiO₂, DCM:Methanol=100:1 to 5:1) to give (2S,4R)-4-fluoro-1-((R)-3,3,3-trifluoro-2-(2-(2-methoxyethoxy)ethoxy)-2-methylpropanoyl)pyrrolidine-2-carboxylic acid (3.12 g, 7.95 mmol, 40.01% yield, 95.668% purity) as a white solid. LCMS (ESI+): m/z 376.0 (M+H)

### Building Block 8: Preparation of (2S,4R)-1-[6,6-difluoro-2-(trifluoromethyl)spiro[3.3]heptane-2-carbonyl]-4-fluoropyrrolidine-2-carboxylic Acid

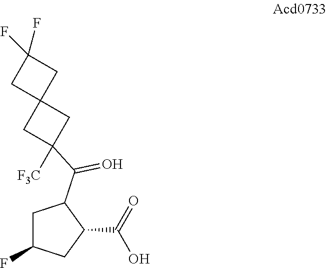 [SMILES: O=C(O)[C@@H]1C[C@@H](F)CC1C(=O)C1(C(F)(F)F)CC2(CC(F)(F)C2)C1]

To a stirred solution of methyl 6,6-difluorospiro[3.3]heptane-2-carboxylate (10 g, 52.579 mmol, 1 equiv) in THF (150 mL) was added LDA (52.58 mL, 105.158 mmol, 2.00 equiv) dropwise at −78° C. under argon atmosphere. The resulting mixture was stirred for 45 min at −78° C. under argon atmosphere and 1-(trifluoromethyl)-1lambda3,2-benziodoxol-3-one (33.23 g, 105.158 mmol, 2 equiv) was added at −78° C. The resulting mixture was stirred for 4 h from −78° C. to room temperature under argon atmosphere. Desired product could be detected by GCMS. Then LiOH (6.30 g, 262.895 mmol, 5 equiv) and H₂O (200 mL) were added dropwise at 0° C. The resulting mixture was stirred for overnight at room temperature. Desired product could be detected by LCMS. The resulting mixture was concentrated under reduced pressure. The aqueous layer was extracted with EtOAc (200 mL). The organic phase was washed with 4×100 mL of 1N NaOH. The mixture was acidified to pH 5 with conc. HCl at 0° C. The aqueous layer was extracted with EtOAc (2×500 mL). The resulting mixture was concentrated under reduced pressure. The resulting mixture was filtered and the filter cake was washed with MeCN (2×200 mL). The filtrate was concentrated under reduced pressure. The crude product (20 g) was purified by Ms guide Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 41% B to 54% B in 7 min, 54% B; Wave Length: 254; 220 nm; RT1(min): 6.140; Number Of Runs: 0) to afford 6,6-difluoro-2-(trifluoromethyl)spiro[3.3]heptane-2-carboxylic acid (900 mg, 6.31%) as a light yellow solid. LCMS: (ESI, m/z): [M+H]⁻=243.

To a stirred solution of 6,6-difluoro-2-(trifluoromethyl)spiro[3.3]heptane-2-carboxylic acid (500 mg, 2.048 mmol, 1.00 equiv) and methyl (2S,4R)-4-fluoropyrrolidine-2-carboxylate (10.85 mg, 0.074 mmol, 1.8 equiv) in MeCN (5 mL) were added TCFH (861.87 mg, 3.072 mmol, 1.5 equiv) and NMI (1261.01 mg, 15.360 mmol, 7.5 equiv) dropwise at 0° C. under argon atmosphere. The resulting mixture was stirred overnight at 50° C. The resulting mixture was concentrated under reduced pressure. The residue was purified by reverse flash chromatography with the following conditions: column, silica gel; mobile phase, MeCN in water (0.1% FA), 40% to 100% gradient in 15 min; detector, UV 210 nm. This resulted in methyl (2S,4R)-1-[6,6-difluoro-2-(trifluoromethyl)spiro[3.3]heptane-2-carbonyl]-4-fluoropyrrolidine-2-carboxylate (350 mg, 42.12%) as a light yellow solid. LCMS: (ESI, m/z): [M+H]⁺=374.

To a stirred solution of methyl (2S,4R)-1-[6,6-difluoro-2-(trifluoromethyl)spiro[3.3]heptane-2-carbonyl]-4-fluoropyrrolidine-2-carboxylate (380 mg, 1.018 mmol, 1.00 equiv) in THF (3 mL)/H₂O (3 mL) was added LiOH (44.91 mg, 1.876 mmol, 2 equiv) at room temperature. The resulting mixture was stirred for overnight at room temperature. Desired product could be detected by LCMS. The resulting mixture was diluted with water (10 mL). The aqueous layer was extracted with EtOAc (10 mL). The mixture/residue was acidified to pH 4 with HCl (aq.). The aqueous layer was extracted with EtOAc (10 mL). The resulting mixture was concentrated under reduced pressure. This resulted in (2S,4R)-1-[6,6-difluoro-2-(trifluoromethyl)spiro[3.3]heptane-2-carbonyl]-4-fluoropyrrolidine-2-carboxylic acid (319.6 mg, 92.51%) as a white solid. LCMS: (ESI, m/z): [M+H]⁻=358.

### Building Block 9: Preparation of (2S,4R)-1-[3,3-difluoro-1-(trifluoromethyl)cyclopentanecarbonyl]-4-fluoropyrrolidine-2-carboxylic Acid

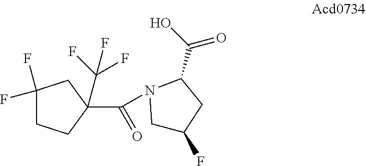 [SMILES: O=C(O)[C@@H]1C[C@@H](F)CN1C(=O)C1(C(F)(F)F)CCC(F)(F)C1]

This compound was prepared following the general synthetic sequence described for the preparation of Building Block 8 using of methyl 3,3-difluorocyclopentane-1-carboxylate instead of methyl 6,6-difluorospiro[3.3]heptane-2-carboxylate. ESI MS m/z 332.

### Building Block 10: Preparation of (2S,4R)-1-[4,4-difluoro-1-(trifluoromethyl)cyclohexanecarbonyl]-4-fluoropyrrolidine-2-carboxylic Acid

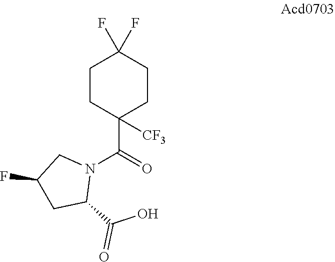 [SMILES: O=C(O)[C@@H]1C[C@@H](F)CN1C(=O)C1(C(F)(F)F)CCC(F)(F)CC1]

This compound was prepared following the general synthetic sequence described for the preparation of Building Block 8 using ethyl 4,4-difluorocyclohexane-1-carboxylate instead of methyl 6,6-difluorospiro[3.3]heptane-2-carboxylate. ESI MS m/z 348.

### Building Block 11: Preparation of (2S,4R)-1-(1-(difluoromethyl)-3,3-difluorocyclobutane-1-carbonyl)-4-fluoropyrrolidine-2-carboxylic Acid

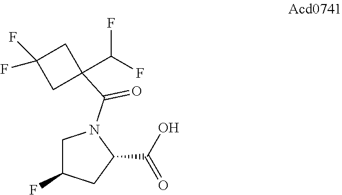 [SMILES: O=C(O)[C@@H]1C[C@@H](F)CN1C(=O)C1(C(F)F)CC(F)(F)C1]

To a stirred solution of 1,1-diisopropyl 3,3-dimethoxycyclobutane-1,1-dicarboxylate (10 g, 34.681 mmol, 1 equiv) in DCM (100 mL) was added DIBAl-H (69.36 mL, 69.362 mmol, 2 equiv, 1M in DCM) dropwise at −78° C. under argon atmosphere. The resulting mixture was stirred for 4 h at −78° C. under argon atmosphere. Desired product could be detected by GCMS. The reaction was quenched with 2 N HCl (aq.) at 0° C. The aqueous layer was extracted with CH₂Cl₂ (2×50 mL). The combined organics were dried over anhydrous Na₂SO₄ and concentrated under reduced pressure. The residue was purified by silica gel column chromatography, eluted with PE/EA (5:1) to afford isopropyl 1-formyl-3,3-dimethoxycyclobutane-1-carboxylate (2.1 g, 24.98%) as a colorless oil. LCMS: (ESI, m/z): [M+H]⁺=230.

The mixture of isopropyl 1-formyl-3,3-dimethoxycyclobutane-1-carboxylate (2.1 g, 9.120 mmol, 1 equiv) in 6N HCl (25 mL) was stirred overnight at room temperature. Desired product could be detected by GCMS. The aqueous layer was extracted with CH₂Cl₂ (50 mL). The organic layer was washed with brine, dried over anhydrous Na2SO4 and was concentrated under reduced pressure. This resulted in isopropyl 1-formyl-3-oxocyclobutane-1-carboxylate (1 g, 53.58%) as a colorless oil. LCMS: (ESI, m/z): [M+H]⁺=184.

To a stirred solution of isopropyl 1-formyl-3-oxocyclobutane-1-carboxylate (1 g, 5.429 mmol, 1 equiv) in DCM (20 mL) was added DAST (4.81 g, 29.860 mmol, 5.5 equiv) dropwise at 0° C. under argon atmosphere. The resulting mixture was stirred overnight at room temperature under argon atmosphere. Desired product could be detected by GCMS. The reaction was quenched by the addition of sat. NaHCO₃ (aq.) (100 mL) at 0° C. The residue was purified by silica gel column chromatography, eluted with CH₂Cl₂ to afford isopropyl 1-(difluoromethyl)-3,3-difluorocyclobutane-1-carboxylate (1 g, 72.65%) as a colorless oil. LCMS: (ESI, m/z): [M+H]⁺=228.

To a stirred solution of isopropyl 1-(difluoromethyl)-3,3-difluorocyclobutane-1-carboxylate (1.5 g, 6.574 mmol, 1 equiv) in THF (20 mL) was added dropwise NaOH (0.79 g, 19.722 mmol, 3 equiv) in H₂O (20 mL) at 0° C. The resulting mixture was stirred overnight at room temperature. Desired product could be detected by LCMS. The resulting mixture was diluted with water (20 mL) and acidified with HCl (aq.) to pH=5. The aqueous layer was extracted with EtOAc (2×30 mL). The combined organics were dried over anhydrous Na₂SO₄ and concentrated under reduced pressure. This resulted in 1-(difluoromethyl)-3,3-difluorocyclobutane-1-carboxylic acid (730 mg, 56.69%) as a colorless oil. LCMS: (ESI, m/z): [M+H]⁻=185.

Into a solution of 1-(difluoromethyl)-3,3-difluorocyclobutane-1-carboxylic acid (1 g, 5.373 mmol, 1 equiv), TCFH (2.26 g, 8.059 mmol, 1.5 equiv) and methyl (2S,4R)-4-fluoropyrrolidine-2-carboxylate (0.87 g, 5.910 mmol, 1.1 equiv) in ACN (20 mL) was added NMI (3.31 g, 40.297 mmol, 7.5 equiv) dropwise at 0° C. under nitrogen atmosphere. The mixture was stirred for 16 h at room temperature. The residue was purified by reverse flash chromatography with the following conditions: column, C18 silica gel; mobile phase, MeCN in Water (0.1% FA), 10% to 50% gradient in 40 min; detector, UV 220 nm. This resulted in methyl (2S,4R)-1-[1-(difluoromethyl)-3,3-difluorocyclobutanecarbonyl]-4-fluoropyrrolidine-2-carboxylate (300 mg, 15.94%) as a brown solid. LCMS: (ESI, m/z): [M+H]⁺=315.24.

Into a solution of methyl (2S,4R)-1-[1-(difluoromethyl)-3,3-difluorocyclobutanecarbonyl]-4-fluoropyrrolidine-2-carboxylate (600 mg, 1.903 mmol, 1 equiv) in THF (10 mL) was added LiOH (136.75 mg, 5.709 mmol, 3 equiv) in H2O (10 mL) at 0° C. under nitrogen atmosphere. The resulting solution was stirred for 16 h at room temperature. The reaction mixture was concentrated in vacuo to remove THF. The aqueous layer was acidified with 1 N HCl to pH=5. The aqueous layer was extracted with EtOAc (3×20 mL). The combined organic layers were washed with brine, dried over Na₂SO₄ and filtered. The filtrate was concentrated in vacuo. This resulted in (2S,4R)-1-[1-(difluoromethyl)-3,3-difluorocyclobutanecarbonyl]-4-fluoropyrrolidine-2-carboxylic acid (0.4957 g, 84.80%) as a white solid. LCMS: (ESI, m/z): [M+H]⁺=301.21

### Building Block 12: Preparation of (2R)-2-[(tert-butoxycarbonyl)amino]-3,3,3-trifluoropropanoic Acid

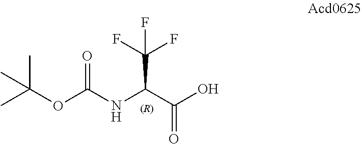

To a stirred solution of trifluoro-D-alanine (700 mg, 4.893 mmol, 1 equiv) and TEA (4.08 mL, 29.358 mmol, 6.0 equiv) in THF (14.00 mL) was added Boc₂O (1.57 mL, 7.339 mmol, 1.5 equiv) in portions at room temperature under nitrogen atmosphere. The resulting mixture was stirred overnight at room temperature under nitrogen atmosphere. The resulting mixture was diluted with EtOAc (15 mL). The organic layer washed with dilute HCl(aq.) (1×15 mL) and water (1×15 mL), dried and concentrated under reduced pressure. The crude product was purified by Prep-HPLC to afford (2R)-2-[(tert-butoxycarbonyl)amino]-3,3,3-trifluoropropanoic acid (0.4712 g, 37.62%) as a white solid. LCMS: (ESI, m/z): [M−H]⁻=242.2.

### Building Block 13: Preparation of (2S)-2-[(tert-butoxycarbonyl)amino]-3,3,3-trifluoropropanoic Acid

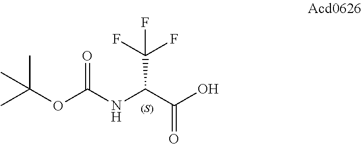

This compound was prepared following the general synthetic sequence described for the preparation of Building Block 12 using trifluoro-L-alanine. ESI MS m/z 242.2

### Building Block 14: Preparation of (R)-2-(difluoromethoxy)-3,3,3-trifluoro-2-methylpropanoic Acid

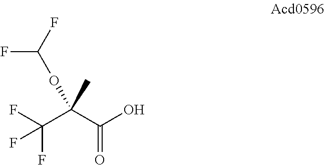

(R)-3,3,3-trifluoro-2-hydroxy-2-methylpropanoic acid (7.0 g, 44.3 mmol) was dissolved in DMF (70 ml), K₂CO₃ (6.7 g, 48.7 mmol) was added and stirred for 10 min. The benzyl bromide (8.34 g, 48.7 mmol) was added and the reaction mixture was stirred at RT for another 4 hours. The mixture was quenched with water (150 mL) and extracted with EtOAc (70 mL×3). The combined organic layers were washed with brine (70 mL×3), dried over anhydrous Na₂SO₄ and then concentrated in vacuo. The residue was purified by column chromatography on silica gel (PE:EtOAc=20:1) to give benzyl (R)-3,3,3-trifluoro-2-hydroxy-2-methylpropanoate (7.7 g, 73%) as a colorless liquid. %). ESI MS m/z: 248.07

Added an aqueous KOH solution (20 wt %, 41 mL, 176.4 mmol) to a mixture of benzyl (R)-3,3,3-trifluoro-2-hydroxy-2-methylpropanoate (7.3 g, 29.4 mmol) and DCM (150 mL) at 0° C. with vigorous stirring, Then a solution of TMSCF₂Br (9.0 g, 44.0 mmol) in DCM (30 mL) was added into the mixture at 0° C., Stirred the mixture at rt for 16 hours. Quenched the reaction mixture by adding water (100 mL), Extracted with CH₂Cl₂ (50 mL×3). Combined the organic layers and dried over anhydrous MgSO₄. Removed the solvents in vacuo, and Purified the residue by Pre-HPLC (Water (0.01 mol/L NH₄HCO₃): ACN=100% to 75%) to obtain product, benzyl (R)-2-(difluoromethoxy)-3,3,3-trifluoro-2-methylpropanoate, (2.2 g, 25%) as a colorless liquid. ESI MS m/z: 298.06

A mixture of benzyl (R)-2-(difluoromethoxy)-3,3,3-trifluoro-2-methylpropanoate (2.2 g, 7.38 mmol) and 10% Pd/C (600 mg) in MeOH (100 mL) was stirred at room temperature for 1 hour under H₂ atmosphere. Then the Pd/C was removed by filtration through a pad of Celite. The filtrate was concentrated in vacuo and the residue was purified by column chromatography on silica gel (DCM:MeOH=100:0 to 50:1) to give the desired product (850 mg, 55%) as a light-brown liquid. ESI MS m/z: 208.06.

### Building Block 15: Preparation of N2-(((9H-fluoren-9-yl)methoxy)carbonyl)-N6-(tert-butoxycarbonyl)-N6-(2,2,2-trifluoroethyl)-L-lysine

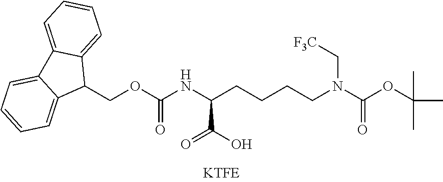 [SMILES: CC(C)(C)OC(=O)N(CCCC[C@H](NC(=O)OCC1c2ccccc2-c2ccccc21)C(=O)O)CC(F)(F)F]

To a dissolved solution of methyl ((benzyloxy)carbonyl)-L-lysinate (5 gr, 16.99 mmol) in THF (84 ml) was added cesium carbonate (16.57 gr, 50.99 mmol) followed by 2,2,2-Trifluoroethyl trifluoromethanesulfonate (2.58 ml, 17.84 mmol). This was allowed to react at 60° C. for 4 hr. Upon completion, the reaction was cooled, quenched with water and extracted 3× with EtOAc. The combined organics was dried over MgSO4, filtered and reduced. The crude was taken onto the next reaction without further purification. ESI MS m/z 464.1

Methyl N2-((benzyloxy)carbonyl)-N6-(2,2,2-trifluoroethyl)-L-lysinate (6.0 g, 12.93 mmol) was dissolved in dioxanes (64 ml) and to this was added a dissolved solution of NaHCO3 (3.25 gr, 38.79 mmol) in water (20 ml). Boc2O (5.5 gr, 25.39 mmol) was added and the reaction was allowed to run at room temperature for 12 h. Upon completion of the reaction, water was added and the organics was extracted with EtOAc 3×. Combined organics was dried over MgSO4, filtered, and reduced. The crude was purified by column chromatography (60% EtOAc/Hex) to afford the desired product (5.7 g, 93%) ESI MS m/z 476.3

Methyl N2-((benzyloxy)carbonyl)-N6-(tert-butoxycarbonyl)-N6-(2,2,2-trifluoroethyl)-L-lysinate (5.2 gr, 10.92 mmol) was dissolved in dioxanes (120 ml). To this, a dissolved solution of lithium hydroxide (895 mg, 21.82 mmol) was added and the reaction was allowed to run at room temperature for 2 h. Afterwards, the reaction was quenched with a saturated solution of citric acid and extracted with EtOAc. The combined organics was dried over MgSO4, filtered, and reduced to afford the crude product which was taken onto the next reaction without further purification. ESI MS m/z 462.20.

N2-((benzyloxy)carbonyl)-N6-(tert-butoxycarbonyl)-N6-(2,2,2-trifluoroethyl)-L-lysine was suspended in MeOH (150 ml). Palladium (10% on carbon, 1.09 mmol, 116 mg) was added and the mixture was stirred under 1 atm of hydrogen for 30 h. The resulting suspension was filtered and reduced. The crude product was redissolved in dioxane (110 ml), and to this a dissolved solution of NaHCO₃ (4.5 g, 53.5 mmol) in water (80 ml) and FMOCOSu (3.8 g, 11.27 mmol) was added. This mixture was allowed to stir for 12 h. Upon completion, a saturated solution of citric acid was added and the organics was extracted 3× with EtOAc. The combined organics was dried over MgSO4, filtered, and reduced. The crude was purified through column chromatography (80% EtOAc) to afford N2-(((9H-fluoren-9-yl)methoxy)carbonyl)-N6-(tert-butoxycarbonyl)-N6-(2,2,2-trifluoroethyl)-L-lysine as a white powder (5.7 g, 95%) after lyophilization, ESI MS m/z 550.18.

### Building Block 16: Preparation of (S)-2-((((9H-fluoren-9-yl)methoxy)carbonyl)amino)-3-(5-chloro-2-fluorophenyl)propanoic Acid

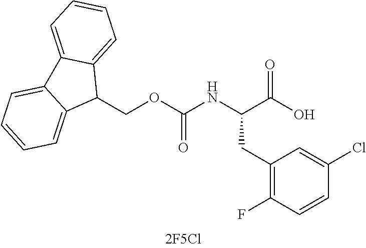 [SMILES: O=C(N[C@@H](Cc1cc(Cl)ccc1F)C(=O)O)OCC1c2ccccc2-c2ccccc21]

### Step 1: Synthesis of (2-chloro-5-fluorophenyl)methanol

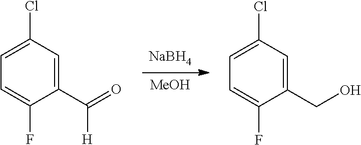 [SMILES: "MeOH" ; O=Cc1cc(Cl)ccc1F ; "NaBH4" ; OCc1cc(Cl)ccc1F]

2-chloro-5-fluorobenzaldehyde (1 g, 6.32 mmol) was dissolved in MeOH (30 ml) and cooled to 0° C. NaBH4 (257 mg, 6.96 mmol) was added in two batches, then the mixture was warmed to room temperature and let run for 1 h. Afterwards the reaction was quenched with 1N HCl and extracted with EtOAc 3×. The combined organics was dried over MgSO4, filtered, and solvent reduced. The crude product was taken onto the next reaction without further purification.

### Step 2: Synthesis of 2-(bromomethyl)-1-chloro-4-fluorobenzene

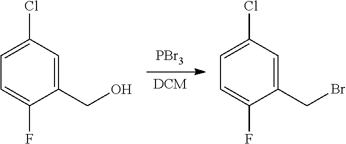

Dissolved (2-chloro-5-fluorophenyl)methanol in DCM (80 ml) and cooled 0° C. To this, phosphorus tribromide (610 ul, 6.32 mmol) was added dropwise. After addition, the reaction was allowed to run at room temperature for 4 h. After completion, the reaction was cooled in an ice bath. Saturated sodium bicarbonate was slowly until the mixture reached a pH of 7. The organics was then extracted with DCM 3×. The combined organics was dried over MgSO4, filtered and the solvent reduced. The crude product was taken onto the next reaction without further purification.

### Step 3: Synthesis of 2-(2-chloro-5-fluorobenzyl)-5-isopropyl-3,6-dimethoxy-2,5-dihydropyrazine

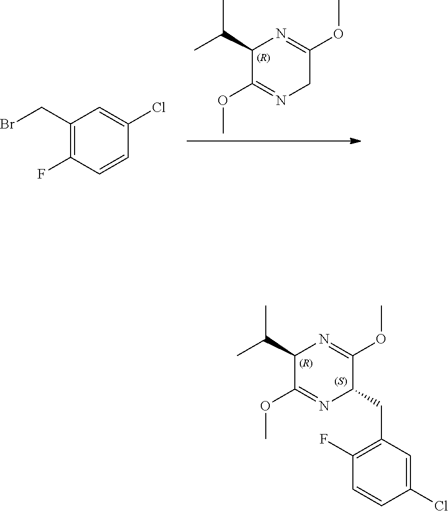

To a three-necked round bottom flask fitted with a thermometer, septum and argon inlet was added (2R)-3,6-dimethoxy-2-(propan-2-yl)-2,5-dihydropyrazine (693 mg, 3.76 mmol). Dry THF (37 ml) was added and the reaction was cooled to −78° C. To this, 2.5M of nBuLi (1.8 ml) was added dropwise. This was allowed to react at −78° C. for 30 min. Then, a dissolved solution of 2-(bromomethyl)-1-chloro-4-fluorobenzene (1.0 g, 4.52 mmol) in THF (20 ml). This reaction was allowed to run for 2 h at −78° C. After, the reaction was quenched with saturated ammonium chloride and extracted with EtOAc 3×. The combined organics was dried over MgSO4, filtered, and solvent reduced. The crude was purified over column chromatography (15% EtOAc/Hex) to afford the desired product, (2S,5R)-2-(5-chloro-2-fluorobenzyl)-5-isopropyl-3,6-dimethoxy-2,5-dihydropyrazine, as a clear oil (1.5 g, 73%), ESI MS m/z 231.05

### Step 4: Synthesis of methyl 2-amino-3-(2-chloro-5-fluorophenyl)propanoate

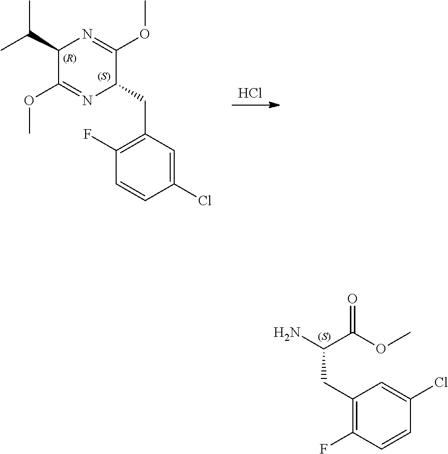

2-(2-chloro-5-fluorobenzyl)-5-isopropyl-3,6-dimethoxy-2,5-dihydropyrazine (1.5 g, 4.60 mmol) was dissolved in THF (50 ml) and cooled to 0° C. in an ice bath. Dropwise, 2N HCL (65 ml) was added. Then the reaction was warmed to room temperature and allowed to react for 2 h. Upon completion the reaction was cooled down in an ice bath, and NH4OH was added until the pH reached 8-9. The reaction was then extracted with EtOAc 3× and combined organics dried over MgSO4, filtered and solvent reduced. The crude product was purified on column chromatography (60% EtOAc/Hex) to provide the desired product, methyl 2-amino-3-(2-chloro-5-fluorophenyl)propanoate as a clear oil. (800 mg, 75%)

### Step 5: Synthesis of (S)-2-((((9H-fluoren-9-yl)methoxy)carbonyl)amino)-3-(5-chloro-2-fluorophenyl)propanoic Acid

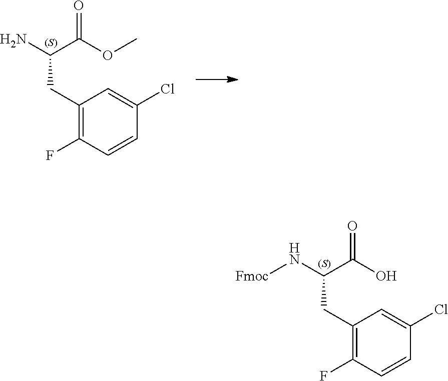

Methyl 2-amino-3-(2-chloro-5-fluorophenyl)propanoate (800 mg, 3.46 mmol) was dissolved in dioxanes (12 ml) and to this was added a dissolved solution of LiOH (290 mg, 6.92 mmol) in water (23 ml). This reaction was allowed to run for 1 h. After, the mixture was cooled in an ice bath and 2N HCl was added until the pH reached 4-5. To this, a dissolved solution of NaHCO3 (1.4 gr, 16.6 mmol) in water (20 ml) was added, followed by a dissolved solution of FmocOSu (1.2 gr, 3.56 mmol) in dioxane (30 ml). This was allowed to react at room temperature for 12 h. The reaction was quenched with 1N HCl, then extracted with EtOAc 3×. The combined organics was dried over MgSO4, filtered, and solvent reduced. The crude product was purified by column chromatography (50% EtOAc) to afford (S)-2-((((9H-fluoren-9-yl)methoxy)carbonyl)amino)-3-(5-chloro-2-fluorophenyl)propanoic acid as a white solid. (1.3 gr, 85%) ESI MS m/z 439.10.

### Building Block 17: Preparation of (S)-2-((((9H-fluoren-9-yl)methoxy)carbonyl)amino)-3-(3,6-dichloro-2-fluorophenyl)propanoic Acid

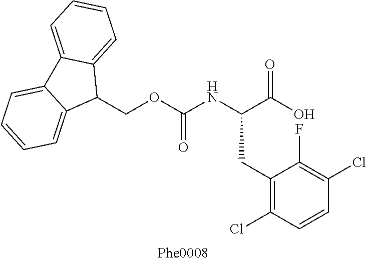 [SMILES: O=C(N[C@@H](Cc1c(Cl)ccc(Cl)c1F)C(=O)O)OCC1c2ccccc2-c2ccccc21]

This compound was prepared following the general synthetic sequence described for the preparation of Building Block 16 using 3,6-dichloro-2-fluorobenzaldehyde as the starting material. ESI MS m/z 473.0.

### Building Block 18: Preparation of (S)-2-((((9H-fluoren-9-yl)methoxy)carbonyl)amino)-3-(2,5-difluorophenyl)propanoic Acid

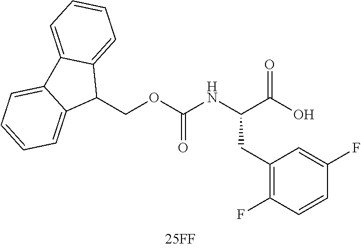 [SMILES: O=C(N[C@@H](Cc1cc(F)ccc1F)C(=O)O)OCC1c2ccccc2-c2ccccc21]

This compound was prepared following the general synthetic sequence described for the preparation of Building Block 16 using 2,5-difluorobenzaldehyde as the starting material. ESI MS m/z 423.13.

### Building Block 19: Preparation of (S)-2-((((9H-fluoren-9-yl)methoxy)carbonyl)amino)-3-(2-chloro-5 fluorophenyl)propanoic Acid

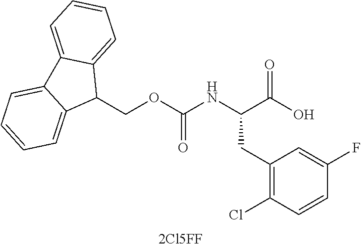 [SMILES: O=C(N[C@@H](Cc1cc(F)ccc1Cl)C(=O)O)OCC1c2ccccc2-c2ccccc21]

This compound was prepared following the general synthetic sequence described for the preparation of Building Block 16 using 2-chloro-5-fluorobenzaldehyde as the starting material. ESI MS m/z 439.10

### Building Block 20: Preparation of (S)-2-((((9H-fluoren-9-yl)methoxy)carbonyl)amino)-3-(5-chloro-2-methylphenyl)propanoic Acid

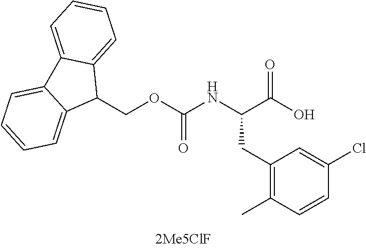 [SMILES: Cc1ccc(Cl)cc1C[C@H](NC(=O)OCC1c2ccccc2-c2ccccc21)C(=O)O]

This compound was prepared following the general synthetic sequence described for the preparation of Building Block 16 using 5-chloro-2-methylbenzaldehyde as the starting material ESI MS m/z 435.12

### Building Block 21: Preparation of (S)-2-((((9H-fluoren-9-yl)methoxy)carbonyl)amino)-3-(5-chloro-2-(trifluoromethoxy)phenyl)propanoic Acid

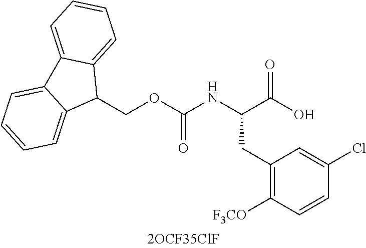 [SMILES: O=C(N[C@@H](Cc1cc(Cl)ccc1OC(F)(F)F)C(=O)O)OCC1c2ccccc2-c2ccccc21]

This compound was prepared following the general synthetic sequence described for the preparation of Building Block 16 using 5-chloro-2-(trifluoromethoxy)benzaldehyde as the starting material. ESI MS m/z 505.09

### Building Block 22: Preparation of (S)-2-((((9H-fluoren-9-yl)methoxy)carbonyl)amino)-3-(5-chloro-2-methoxyphenyl)propanoic Acid

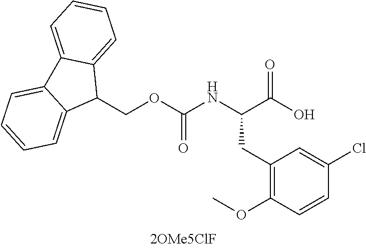 [SMILES: COc1ccc(Cl)cc1C[C@H](NC(=O)OCC1c2ccccc2-c2ccccc21)C(=O)O]

This compound was prepared following the general synthetic sequence described for the preparation of Building Block 16 using 5-chloro-2-methoxybenzaldehyde as the starting material. ESI MS m/z 451.12

### Building Block 23: Preparation of (S)-2-((((9H-fluoren-9-yl)methoxy)carbonyl)amino)-3-(5-bromo-2-chlorophenyl)propanoic Acid

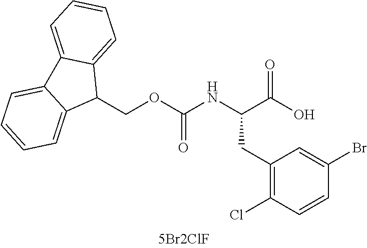 [SMILES: O=C(N[C@@H](Cc1cc(Br)ccc1Cl)C(=O)O)OCC1c2ccccc2-c2ccccc21]

This compound was prepared following the general synthetic sequence described for the preparation of Building Block 16 using 5-bromo-2-chlorobenzaldehyde as the starting material. ESI MS m/z 499.02

### Building Block 24: Preparation of (S)-2-((((9H-fluoren-9-yl)methoxy)carbonyl)amino)-3-(2-bromo-5-chlorophenyl)propanoic Acid

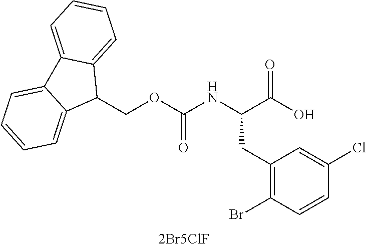 [SMILES: O=C(N[C@@H](Cc1cc(Cl)ccc1Br)C(=O)O)OCC1c2ccccc2-c2ccccc21]

This compound was prepared following the general synthetic sequence described for the preparation of Building Block 16 using 2-bromo-5-chlorobenzaldehyde as the starting material. ESI MS m/z 499.02

### Building Block 25: Preparation of (S)-2-((((9H-fluoren-9-yl)methoxy)carbonyl)amino)-3-(5-chloro-2-iodophenyl)propanoic Acid

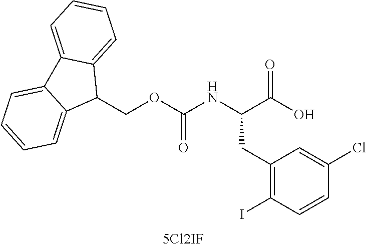 [SMILES: O=C(N[C@@H](Cc1cc(Cl)ccc1I)C(=O)O)OCC1c2ccccc2-c2ccccc21]

This compound was prepared following the general synthetic sequence described for the preparation of Building Block 16 using 5-chloro-2-iodobenzaldehyde as the starting material. ESI MS m/z 547.00

### Building Block 26: Preparation of (S)-2-((((9H-fluoren-9-yl)methoxy)carbonyl)amino)-3-(5-chloro-2-(difluoromethoxy)phenyl)propanoic Acid

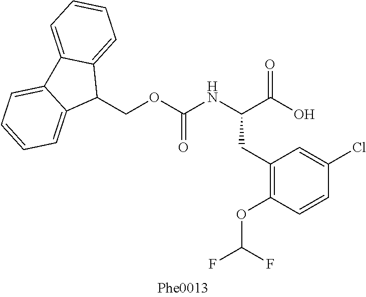 [SMILES: O=C(N[C@@H](Cc1cc(Cl)ccc1OC(F)F)C(=O)O)OCC1c2ccccc2-c2ccccc21]

This compound was prepared following the general synthetic sequence described for the preparation of Building Block 16 using 5-chloro-2-(difluoromethoxy)benzaldehyde as the starting material. ESI MS m/z 487.10

### Building Block 27: Preparation of (2S)-3-(3,3-difluorocyclobutyl)-2-{[(9H-fluoren-9-ylmethoxy)carbonyl]amino} propanoic Acid

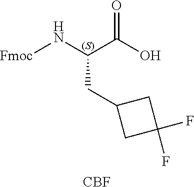

This compound was prepared following the general synthetic sequence described for the preparation of Building Block 16, steps 3 to 5, where 3-(bromomethyl) 1,1-difluorocyclobutane was used instead of 2-(bromomethyl)-1-chloro-4-fluorobenzene. ESI MS m/z 402.3

### Building Block 28: Preparation of (S)-2-((((9H-fluoren-9-yl)methoxy)carbonyl)amino)-3-(5-chloro-2-(cyclopropylmethoxy)phenyl)propanoic Acid

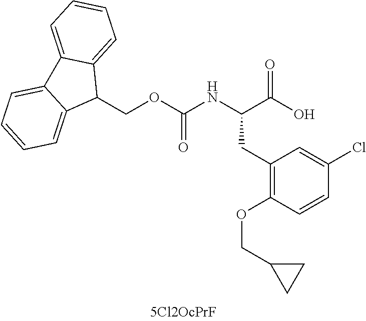 [SMILES: O=C(N[C@@H](Cc1cc(Cl)ccc1OCC1CC1)C(=O)O)OCC1c2ccccc2-c2ccccc21]

### Step 1: Synthesis of 5-chloro-2-(cyclopropylmethoxy)benzaldehyde

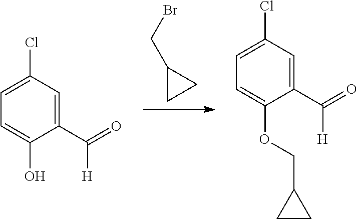

To a dissolved solution of 5-chloro-2-hydroxybenzaldehyde (1.5 gr, 9.61 mmol) in DMF (20 ml) was added K2CO3 (2.0 gr, 14.4 mmol). This was allowed to react for 10 minutes and then bromomethylcyclopropane (2.5 gr, 15.3 mmol) was added. This was allowed to react at room temperature overnight. Upon completion the mixture was quenched with water and the organics was extracted with DCM 3×. The combined organics was dried over MgSO4, filtered and solvent reduced. The crude product was purified by column chromatography (15% EtOAc/Hexanes) to afford 5-chloro-2-(cyclopropylmethoxy)benzaldehyde as a clear oil (1.8 gr, 90%). ESI MS m/z 210.04.

### Step 2: Synthesis of 2-(bromomethyl)-4-chloro-1-(cyclopropylmethoxy)benzene

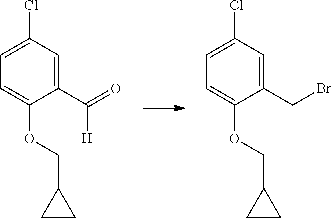 [SMILES: O=Cc1cc(Cl)ccc1OCC1CC1 ; Clc1ccc(OCC2CC2)c(CBr)c1]

5-chloro-2-(cyclopropylmethoxy)benzaldehyde (2.1 gr, 10.0 mmol) was dissolved in EtOH (0.5M) and the mixture was cooled in an ice bath to 0° C. Sodium borohydride (407 mg, 11 mmol) was added in three portions. The mixture was then warmed to room temperature and this was allowed to react for 1 h. Upon completion the solvent was reduced and redissolved in DCM. 1M HCl was added and the organics was extracted with DCM 3×. Combined organics was dried over MgSO4, filtered, and solvent was reduced to afford crude (5-chloro-2-(cyclopropylmethoxy)phenyl)methanol which was taken on to the next step without further purification.

(5-chloro-2-(cyclopropylmethoxy)phenyl)methanol (2.1 gr, 9.9 mmol) was dissolved in DCM (40 ml) and cooled in an ice bath to 0° C. Phosphorus tribromide (2.7 gr, 9.9 mmol) was added dropwise and the mixture was warmed to room temperature. This reaction was allowed to run for 4 h. Upon completion the reaction was cooled in an ice bath and a cold solution of saturated NaHCO3 was added until pH was 7. The mixture was extracted with DCM 3× and combined organics was dried over MgSO4, dried, and the solvent reduced. The crude product, 2-(bromomethyl)-4-chloro-1-(cyclopropylmethoxy)benzene was taken onto the next step without further purification.

### Step 3: Synthesis of tert-butyl (S)-2-((((9H-fluoren-9-yl)methoxy)carbonyl)amino)-3-(5-chloro-2-(cyclopropylmethoxy)phenyl)propanoate

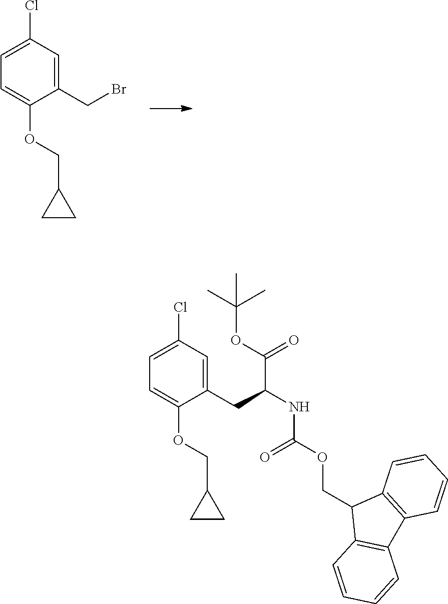 [SMILES: Clc1ccc(OCC2CC2)c(CBr)c1 ; CC(C)(C)OC(=O)[C@H](Cc1cc(Cl)ccc1OCC1CC1)NC(=O)OCC1c2ccccc2-c2ccccc21]

To a 100 ml round bottom flask was added O-Allyl-N-(9-anthracenylmethyl)cinchonidinium bromide (487 mg, 0.805 mmol) and N-(Diphenylmethylene)glycine tert-butyl ester (2.2 g, 8.05 mmol). This was dissolved in DCM and the mixture was cooled to −20° C. To this was added 2-(bromomethyl)-4-chloro-1-(cyclopropylmethoxy)benzene (2.5 g, 9.15 mmol), followed by 45% aqueous KOH (4.35 ml). The reaction was allowed to run for 16 hours at −20° C. Afterwards, water was added and the organics was extracted with DCM 3×. The combined organics was dried over MgSO4, filtered and reduced. The crude was then redissolved in dioxanes. 2N HCl (20 ml) was added dropwise and the reaction was allowed to stir at room temperature for 1 h. Upon completion the mixture was cooled in an ice bath, and saturated NaHCO₃ was added until pH was 7-9. Then, a dissolved solution of FmocOSu (2.8 g, 8.30 mmol) was added and the mixture and allowed to react for 12 h. The solution was quenched with water and organics extracted with EtOAc 3×. Combined organics was dried over MgSO4, filtered, and solvent reduced. The crude was purified over column chromatography (25-50% EtOAc/hexanes) to afford the desired product, tert-butyl (S)-2-((((9H-fluoren-9-yl)methoxy)carbonyl)amino)-3-(5-chloro-2-(cyclopropylmethoxy)phenyl)propanoate (3.5 gr, 85%) as a clear oil. ESI MS m/z 547.2.

### Step 4: Synthesis of (S)-2-((((9H-fluoren-9-yl)methoxy)carbonyl)amino)-3-(5-chloro-2-(cyclopropylmethoxy)phenyl)propanoic Acid

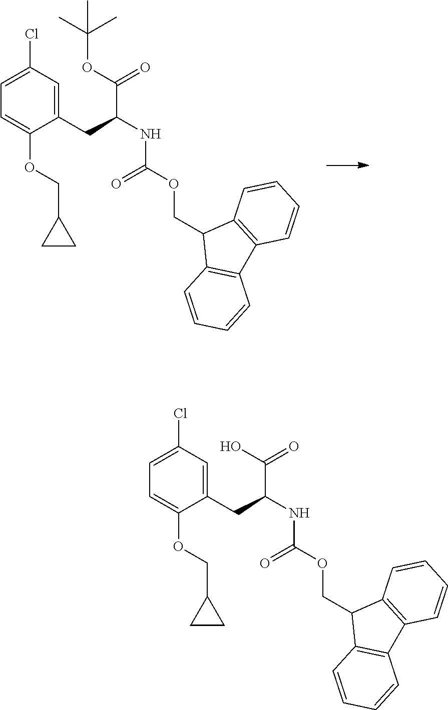 [SMILES: CC(C)(C)OC(=O)[C@H](Cc1cc(Cl)ccc1OCC1CC1)NC(=O)OCC1c2ccccc2-c2ccccc21 ; O=C(N[C@@H](Cc1cc(Cl)ccc1OCC1CC1)C(=O)O)OCC1c2ccccc2-c2ccccc21]

The starting material, tert-butyl (S)-2-((((9H-fluoren-9-yl)methoxy)carbonyl)amino)-3-(5-chloro-2-(cyclopropylmethoxy)phenyl)propanoate (3.5 gr, 6.39 mmol) was dissolved in DCM (20 ml) and to this, a 50% TFA in DCM (30 ml) was added and the reaction was allowed to run at room temperature until complete. Afterwards, the solvent was reduced and the crude was purified over column chromatography (80% EtOAc/Hexanes) to afford the desired product (S)-2-((((9H-fluoren-9-yl)methoxy)carbonyl)amino)-3-(5-chloro-2-(cyclopropylmethoxy)phenyl)propanoic acid (3.1 gr, 100%) as a white solid. ESI MS m/z 491.1.

### Building Block 29: Preparation of (S)-2-((((9H-fluoren-9-yl)methoxy)carbonyl)amino)-3-(5-chloro-2-phenoxyphenyl)propanoic Acid

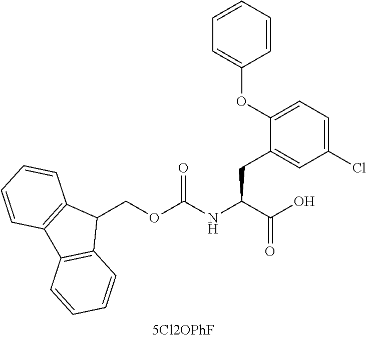 [SMILES: O=C(N[C@@H](Cc1cc(Cl)ccc1Oc1ccccc1)C(=O)O)OCC1c2ccccc2-c2ccccc21]

To a dissolved solution of 5-chloro-2-fluorobenzaldehyde (1.0 gr, 6.32 mmol) in DMF (20 ml) was added K2CO3 (3.93 gr, 28.4 mmol). This was allowed to react for 10 minutes and then phenol (0.89 gr, 9.48 mmol) was added. This was allowed to react at 110° overnight. Upon completion the mixture was quenched with water and the organics was extracted with DCM 3×. The combined organics was dried over MgSO4, filtered and solvent reduced. The crude product was purified by column chromatography (15% EtOAc/Hexanes) to afford 5-chloro-2-phenoxybenzaldehyde as a clear oil (1.0 gr, 68%). ESI MS m/z 232.03.

Building Block 29 was prepared from 5-chloro-2-phenoxybenzaldehyde following the general synthetic sequence described for the preparation of Building Block 28, steps 2 to 4. ESI MS m/z 513.13

### Building Block 30: Preparation of (S)-2-((((9H-fluoren-9-yl)methoxy)carbonyl)amino)-3-(5-chloro-2-(cyclopentylmethoxy)phenyl)propanoic Acid

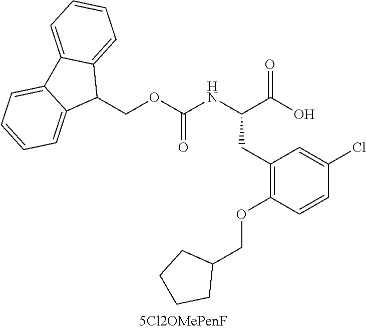 [SMILES: O=C(N[C@@H](Cc1cc(Cl)ccc1OCC1CCCC1)C(=O)O)OCC1c2ccccc2-c2ccccc21]

This compound was prepared following the general synthetic sequence described for the preparation of Building Block 28 using (bromomethyl)cyclopentane instead of bromomethylcyclopropane. ESI MS m/z 519.18

### Building Block 31: Preparation of (S)-2-((((9H-fluoren-9-yl)methoxy)carbonyl)amino)-3-(5-chloro-2-(cyclopentyloxy)phenyl)propanoic Acid

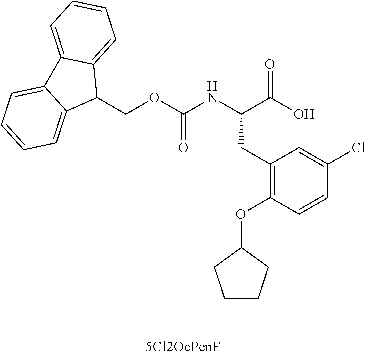 [SMILES: O=C(N[C@@H](Cc1cc(Cl)ccc1OC1CCCC1)C(=O)O)OCC1c2ccccc2-c2ccccc21]

This compound was prepared following the general synthetic sequence described for the preparation of Building Block 28 using bromocyclopentane instead of bromomethylcyclopropane. ESI MS m/z 505.17

### Building Block 32: Preparation of (S)-2-((((9H-fluoren-9-yl)methoxy)carbonyl)amino)-3-(5-chloro-2-(cyclohexyloxy)phenyl)propanoic Acid

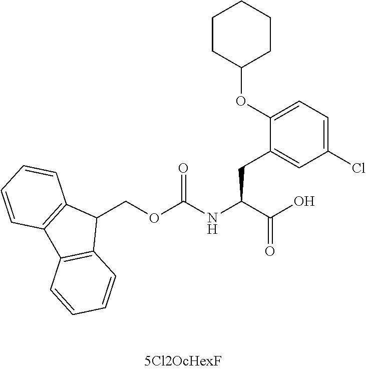 [SMILES: O=C(N[C@@H](Cc1cc(Cl)ccc1OC1CCCCC1)C(=O)O)OCC1c2ccccc2-c2ccccc21]

This compound was prepared following the general synthetic sequence described for the preparation of Building Block 28 using bromocyclohexane instead of bromomethylcyclopropane. ESI MS m/z 519.17

### Building Block 33: Preparation of (S)-2-((((9H-fluoren-9-yl)methoxy)carbonyl)amino)-3-(5-chloro-2-(2,2,2-trifluoroethoxy)phenyl)propanoic Acid

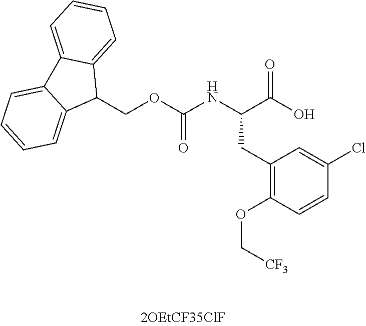 [SMILES: O=C(N[C@@H](Cc1cc(Cl)ccc1OCC(F)(F)F)C(=O)O)OCC1c2ccccc2-c2ccccc21]

This compound was prepared following the general synthetic sequence described for the preparation of Building Block 28 but with 2,2,2-Trifluoroethyl trifluoromethanesulfonate instead of bromomethylcyclopropane. ESI MS m/z 519.11

### Building Block 34: Preparation of (S)-2-((((9H-fluoren-9-yl)methoxy)carbonyl)amino)-3-(5-chloro-2-(cyclobutylmethoxy)phenyl)propanoic Acid

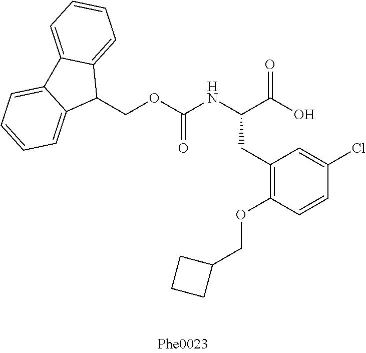 [SMILES: O=C(N[C@@H](Cc1cc(Cl)ccc1OCC1CCC1)C(=O)O)OCC1c2ccccc2-c2ccccc21]

A mixture of 4-chloro-2-iodophenol (6 g, 23.580 mmol, 1 equiv) and K₂CO₃ (9.85 g, 70.740 mmol, 3 equiv) in DMF (50 mL) was treated with (bromomethyl)cyclobutane (4.22 g, 28.296 mmol, 1.2 equiv) and stirred for 2 h at 100° C. under nitrogen atmosphere. The reaction was diluted with water and extracted with EtOAc (100 mL×3). The combined organic layers were washed with brine (50 mL×3) and dried over anhydrous Na₂SO₄. After filtration, the filtrate was concentrated under reduced pressure. The residue was purified by silica gel column chromatography, eluted with PE/EA (20/1-5/1) to afford 4-chloro-1-(cyclobutylmethoxy)-2-iodobenzene (7.3 g, 95.97%) as a white solid. No MS signal was found on LCMS.

To a stirred mixture of 4-chloro-1-(cyclobutylmethoxy)-2-iodobenzene (4 g, 12.400 mmol, 1 equiv), CuI (0.05 g, 0.248 mmol, 0.02 equiv) and Pd(dppf)Cl2CH2Cl2 (0.10 g, 0.124 mmol, 0.01 equiv) in DMA (30 mL) was added methyl (2R)-2-[(tert-butoxycarbonyl)amino]-3-(iodozincio)propanoate (24.80 mL, 24.800 mmol, 2.0 equiv) dropwise at 20° C. under nitrogen atmosphere. The resulting mixture was stirred for 2 h at 80° C. under nitrogen atmosphere. The reaction was purified directly by reverse flash chromatography with the following conditions: column, C18 silica gel; mobile phase, MeCN in Water (0.1% FA), 10% to 60% gradient in 10 min; detector, UV 210 nm. This resulted in methyl (2S)-2-[(tert-butoxycarbonyl)amino]-3-[5-chloro-2-(cyclobutylmethoxy)phenyl]propanoate (3.1 g, 62.83%) as a dark brown oil. LCMS: (ESI, m/z): [M+Na]⁺=420.

To a stirred mixture of methyl (2S)-2-[(tert-butoxycarbonyl)amino]-3-(5-chloro-2-cyclobutoxyphenyl)propanoate (2.9 g, 7.555 mmol, 1 equiv) in THF (30 mL) was added aqueous solution of sodium hydroxide (1.51 g, 37.775 mmol, 5 equiv) dropwise at 0° C. The resulting mixture was stirred for additional 12 h at room temperature. The reaction was acidified with HCl (1N) to pH=5. The resulting mixture was extracted with EtOAc (2*50 mL). The combined organic layers were washed with brine (1*30 mL) and dried over anhydrous Na₂SO₄. After filtration, the filtrate was concentrated under reduced pressure. This resulted in (2S)-2-[(tert-butoxycarbonyl)amino]-3-(5-chloro-2-cyclobutoxyphenyl)propanoic acid (2.3129 g, 82.78%) as a white solid. LCMS: (ESI, m/z): [M+Na]⁺=406.20.

A mixture of (2S)-2-[(tert-butoxycarbonyl)amino]-3-[5-chloro-2-(cyclobutylmethoxy)phenyl]propanoic acid (2.2 g, 5.731 mmol, 1 equiv) in HCl (4M in EtOAc) was stirred for 12 h at room temperature under nitrogen atmosphere. The resulting mixture was concentrated under vacuum. This resulted in (2S)-2-amino-3-[5-chloro-2-(cyclobutylmethoxy)phenyl]propanoic acid (2.0688 g, 127.22%) as a white solid. LCMS: (ESI, m/z): [M+H]⁺=283.90.

To a stirred mixture of (2S)-2-amino-3-[5-chloro-2-(cyclobutylmethoxy)phenyl]propanoic acid (1.6 g, 5.639 mmol, 1 equiv) and NaHCO₃ (2.37 g, 28.195 mmol, 5 equiv) in 1,4-dioxane:H2O (3:1, 50 mL) was added 2,5-dioxopyrrolidin-1-yl 9H-fluoren-9-ylmethyl carbonate (2.28 g, 6.767 mmol, 1.2 equiv) in portions at 0° C. The resulting mixture was stirred for additional 12 h at room temperature. The reaction was acidified with HCl (aq. 1N) to pH=5. The resulting mixture was extracted with EtOAc (3×50 mL). The combined organic layers were dried over anhydrous Na₂SO₄. After filtration, the filtrate was concentrated under reduced pressure. The residue was purified by reverse flash chromatography with the following conditions: column, silica gel; mobile phase, MeCN in water, 0% to 100% gradient in 40 min; detector, UV 254 nm. This resulted in (2S)-3-[5-chloro-2-(cyclobutylmethoxy)phenyl]-2-{[(9H-fluoren-9-ylmethoxy)carbonyl]amino}propanoic acid (1.3205 g, 46.28%) as a white solid. LCMS: (ESI, m/z): [M+H]⁺=506.15.

### Building Block 35: Preparation of (S)-2-((((9H-fluoren-9-yl)methoxy)carbonyl)amino)-3-(5-chloro-2-cyclobutoxyphenyl)propanoic Acid

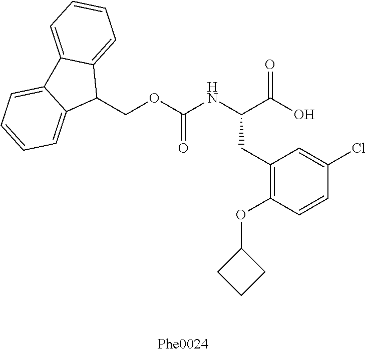 [SMILES: O=C(N[C@@H](Cc1cc(Cl)ccc1OC1CCC1)C(=O)O)OCC1c2ccccc2-c2ccccc21]

This compound was prepared following the general synthetic sequence described for the preparation of Building Block 34 using bromocyclobutane instead of (bromomethyl)cyclobutane. ESI MS m/z 492.1

### Building Block 36: Preparation of (S)-2-((((9H-fluoren-9-yl)methoxy)carbonyl)amino)-3-(5-chloro-2-cyclopropoxyphenyl)propanoic Acid

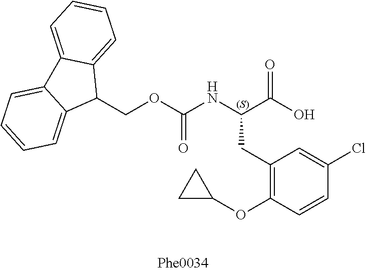

To a stirred mixture of methyl 5-chloro-2-hydroxybenzoate (10 g, 53.593 mmol, 1 equiv) and K₂CO₃ (14.81 g, 107.186 mmol, 2 equiv) in DMF was added 2-chloroethyl p-tosylate (13.84 g, 58.952 mmol, 1.1 equiv) dropwise at room temperature under nitrogen atmosphere. The resulting mixture was stirred for 16 h at 50° C. under nitrogen atmosphere. The mixture was allowed to cool down to room temperature. The resulting mixture was diluted with water (300 mL) and extracted with EtOAc (3×100 mL). The combined organic layers were washed with NH₄Cl (3×150 mL), NH₄HCO₃ (1×150 Ml) and brine (1×150 mL) in sequence and dried over anhydrous Na₂SO₄. After filtration, the filtrate was concentrated in vacuo. The residue was purified by silica gel column chromatography, eluted with PE/EtOAc (10:1) to afford methyl 5-chloro-2-(2-chloroethoxy)benzoate (13 g, 97.38%) as an off-white solid. LCMS: (ESI, m/z): [M+H]⁺=249.00.

To a stirred solution of methyl 5-chloro-2-(2-chloroethoxy)benzoate (13 g, 52.190 mmol, 1 equiv) in THF was added t-BuOK (65.24 mL, 65.240 mmol, 1.25 equiv) dropwise at 0° C. The resulting mixture was stirred for 16 h at room temperature. The reaction was diluted with water (200 mL) and extracted with EtOAc (2×150 mL). The combined organic layers were washed with brine (1×200 mL) and dried over anhydrous Na₂SO₄. After filtration, the filtrate was concentrated under vacuum. The residue was purified by silica gel column chromatography, eluted with PE/EtOAc (10:1) to afford methyl 5-chloro-2-(ethenyloxy) benzoate (6.9 g, 51.61%) as a colorless oil.

The aqueous layer was acidified to pH 3 with HCl and extracted with EtOAc (3×100 mL). The combined organic layers were washed with brine (1×200 mL), dried over anhydrous Na₂SO₄. After filtration, the filtrate was concentrated in vacuo and the residue was reclaimed to react with CH₃I to get another batch of product.

A solution of methyl 5-chloro-2-(ethenyloxy)benzoate (6.9 g, 32.451 mmol, 1 equiv) in CH₂Cl₂ was treated with chloro(iodo)methane (17.17 g, 97.353 mmol, 3 equiv) for 20 min at 0° C. under nitrogen atmosphere followed by the addition of diethylzinc (48.68 mL, 48.677 mmol, 1.5 equiv) dropwise at 0° C. The resulting mixture was stirred for 3 h at room temperature under nitrogen atmosphere. The reaction was quenched with NH₄Cl (100 mL) and NH₃. H₂O (10 mL) at 0° C. The resulting mixture was diluted with water (100 mL) and extracted with CH₂Cl₂ (2×100 mL). The combined organic layers were washed with brine (1×200 mL) and dried over anhydrous Na₂SO₄. After filtration, the filtrate was concentrated in vacuo. The residue was purified by silica gel column chromatography, eluted with PE/EtOAc (7:1) to afford methyl 5-chloro-2-cyclopropoxybenzoate (6.25 g, 84.97%) as a light green oil.

To a stirred solution of methyl 5-chloro-2-cyclopropoxybenzoate (6.25 g, 27.574 mmol, 1 equiv) in toluene (130 mL) was added DIBAl-H (46.08 mL, 227.124 mmol) dropwise at −78° C. under nitrogen atmosphere. The resulting mixture was stirred for 2 h at room temperature and then quenched with NH₄Cl at 0° C. and diluted with water (200 mL). Then the mixture was acidified with diluted HCl (1N) to pH 5 and extracted with EtOAc (3×100 mL). The combined organic layers were washed with brine (1×100 mL) and dried over anhydrous Na₂SO₄. After filtration, the filtrate was concentrated under reduced pressure. The residue was purified by silica gel column chromatography, eluted with PE/EtOAc (5:1) to afford (5-chloro-2-cyclopropoxyphenyl)methanol (4.9 g, 89.45%) as a light brown solid.

To a stirred solution of (5-chloro-2-cyclopropoxyphenyl) methanol (3.73 g, 18.777 mmol, 1 equiv) in DCM (37 mL) was added PBr₃ (7.62 g, 28.166 mmol, 1.5 equiv) dropwise at 0° C. under N₂ atmosphere. The mixture was stirred for 2 h at 0° C. and then neutralized with NaHCO₃ to pH=7. The resulting mixture was extracted with EtOAc (4×200 mL). The combined organic layers were dried over anhydrous Na₂SO₄. After filtration, the filtrate was concentrated under reduced pressure. The residue was purified by silica gel column chromatography, eluted with PE:EtOAc (80:1) to afford 2-(bromomethyl)-4-chloro-1-cyclopropoxybenzene (3.35 g, 68.22%) as a white oil.

A solution of (3R)-3-isopropyl-2,5-dimethoxy-3,6-dihydropyrazine (2.60 g, 14.090 mmol, 1.1 equiv) in THF (33 mL) was treated with n-BuLi (7.8 mL, 82.797 mmol, 6.46 equiv) for 0.5 h at −78° C. under nitrogen atmosphere and the resulting solution was stirred for 1 h at −78° C. To the above solution was added 2-(bromomethyl)-4-chloro-1-cyclopropoxybenzene (3.35 g, 12.809 mmol, 1 equiv) dropwise at −78° C. The mixture was stirred for 2 h at −78° C. and then quenched with NH₄Cl at −78° C. The resulting mixture was extracted with EtOAc (3×100 mL). The combined organic layers were dried over anhydrous Na₂SO₄. After filtration, the filtrate was concentrated under reduced pressure. The residue was purified by silica gel column chromatography, eluted with PE:EtOAc (80:1) to afford (2S,5R)-2-[(5-chloro-2-cyclopropoxyphenyl)methyl]-5-isopropyl-3,6-dimethoxy-2,5-dihydropyrazine (3.06 g, 65.48%) as a white oil. LCMS: (ESI, m/z): [M+H]⁺=365.40.

To a stirred solution of (2S,5R)-2-[(5-chloro-2-cyclopropoxyphenyl)methyl]-5-isopropyl-3,6-dimethoxy-2,5-dihydropyrazine (3.1 g, 8.496 mmol, 1 equiv) in THF (30 mL, 370.283 mmol, 43.58 equiv) was added HCl (2M) (8.5 mL) at rt. The mixture was stirred for 2 h at rt and then neutralized with saturated NaHCO₃ to pH=7. The resulting mixture was extracted with EtOAc (3×50 mL). The combined organic layers were dried over anhydrous Na₂SO₄. After filtration, the filtrate was concentrated under reduced pressure. The resulting mixture was concentrated under vacuum. The residue was purified by reverse flash chromatography with the following conditions: column, C18 silica gel; mobile phase, water in ACN, 0% to 100% gradient in 30 min; detector, UV 254 nm. This resulted in methyl (2S)-2-amino-3-(5-chloro-2-cyclopropoxyphenyl)propanoate (1.9 g, 82.91%) as a white oil. LCMS: (ESI, m/z): [M+H]⁺=270.10.

To a stirred solution of methyl (2S)-2-amino-3-(5-chloro-2-cyclopropoxyphenyl)propanoate (920 mg, 3.411 mmol, 1 equiv) in MeOH (5 mL) was added NaOH (682.11 mg, 17.055 mmol, 5 equiv) in H₂O (5 mL) dropwise at rt. The mixture was stirred for 1 h at rt and then acidified with diluted HCl (1N) to pH=2. The resulting mixture was concentrated under reduced pressure to afford crude product which was used in the next step directly without further purification. LCMS: (ESI, m/z): [M+H]⁺=256.20.

To a stirred solution of (2S)-2-amino-3-(5-chloro-2-cyclopropoxyphenyl) propanoic acid (850 mg, 3.324 mmol, 1 equiv) in 1,4-dioxane (30 mL)/water (10 mL) was added 2,5-dioxopyrrolidin-1-yl 9H-fluoren-9-ylmethyl carbonate (1143.77 mg, 3.390 mmol, 1.02 equiv) and NaHCO₃ (1396.27 mg, 16.620 mmol, 5 equiv) in portions at rt. The mixture was stirred for 2 h at rt and then acidified with diluted HCl (1N) to pH=2. The resulting mixture was extracted with EtOAc (5×50 mL). The combined organic layers were dried over anhydrous Na₂SO₄. After filtration, the filtrate was concentrated under reduced pressure. The residue was purified by reverse flash chromatography with the following conditions: column, C18 silica gel; mobile phase, H₂O in ACN, 0% to 100% gradient in 20 min; detector, UV 254 nm. The resulting mixture was concentrated under reduced pressure. This resulted in (2R)-3-(5-chloro-2-cyclopropoxyphenyl)-2-{[(9H-fluoren-9-ylmethoxy)carbonyl]amino}propanoic acid (1.1361 g, 71.51%) as a white solid. LCMS: (ESI, m/z): [M+Na]⁺=500.10.

### Building Block 37: Preparation of (S)-2-((((9H-fluoren-9-yl)methoxy)carbonyl)amino)-3-(5-chloro-2-(cyclopropylmethoxy)pyridin-3-yl)propanoic Acid

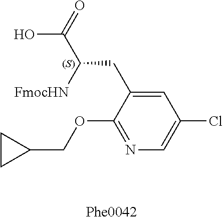

To a stirred mixture of 5-chloro-3-iodopyridin-2-ol (3.2 g, 12.527 mmol, 1 equiv) and Ag₂CO₃ (4.15 g, 15.032 mmol, 1.2 equiv) in toluene was added (bromomethyl)cyclopropane (3.38 g, 25.054 mmol, 2 equiv) dropwise at room temperature under nitrogen atmosphere. The resulting mixture was stirred for additional 3-4 h at 100° C. The reaction was cooled to room temperature and quenched with water at 0° C. The resulting mixture was extracted with EtOAc (3×mL). The organic layer was washed with brine, dried over anhydrous Na₂SO₄ and concentrated under reduced pressure. The residue was purified by silica gel column chromatography, eluted with PE/EA (5:1) to afford 5-chloro-2-(cyclopropylmethoxy)-3-iodopyridine (3.6 g, 92.84%) as a colorless oil. LCMS: (ESI, m/z): [M+H]⁺=310

A solution of 5-chloro-2-(cyclopropylmethoxy)-3-iodopyridine (5 g, 16.154 mmol, 1 equiv) in DMA was treated with copper(I) iodide (0.62 g, 3.231 mmol, 0.2 equiv) and Pd(dppf)Cl₂ (2.36 g, 3.231 mmol, 0.2 equiv) for 2 min at room temperature under nitrogen atmosphere followed by the addition of methyl 2-[(tert-butoxycarbonyl)amino]-3-zinciopropanoate (3 mL, 9.692 mmol, 1.5 equiv, prepared from iodide and Zn powder) dropwise at room temperature. The resulting mixture was stirred for additional 2-3 h at 80° C. The reaction was quenched with water at 0° C. The resulting mixture was extracted with EtOAc (5×mL). The organic layer was washed with brine, dried over anhydrous Na₂SO₄. After filtration, the filtrate was concentrated under reduced pressure. The residue was purified by silica gel column chromatography, eluted with PE/EA (5:1) to afford methyl (2S)-2-[(tert-butoxycarbonyl)amino]-3-[5-chloro-2-(cyclopropylmethoxy)pyridin-3-yl]propanoate (6.4 g, 102.95%) as a crude white solid. LCMS: (ESI, m/z): [M+Na]⁺=385.

To a stirred solution/mixture of methyl (2S)-2-[(tert-butoxycarbonyl)amino]-3-[5-chloro-2-(cyclopropylmethoxy)pyridin-3-yl]propanoate (6.4 g, 16.629 mmol, 1 equiv) in 20 mL of THF was added aqueous solution of sodium hydroxide (NaOH (3.33 g, 83.145 mmol, 5 equiv) in 20 mL of water) at 0° C. under nitrogen atmosphere. The resulting mixture was stirred for additional 1-2 h at room temperature. The reaction was acidified to pH=4 with dilute HCl. The resulting mixture was extracted with EtOAc (50×3 mL). The combined organic layers were washed with brine, dried over anhydrous Na₂SO₄ and concentrated under reduced pressure. The residue was purified by reverse flash chromatography with the following conditions: column, C18 silica gel; mobile phase, H2O in ACN, 10% to 50% gradient in 10 min; detector, UV 254 nm. This resulted in (2S)-2-[(tert-butoxycarbonyl)amino]-3-[5-chloro-2-(cyclopropylmethoxy)pyridin-3-yl]propanoic acid (4.8 g, 77.84%) as a yellow solid. LCMS: (ESI, m/z): [M+H]⁺=371.

Into a solution of (2S)-2-[(tert-butoxycarbonyl)amino]-3-[5-chloro-2-(cyclopropylmethoxy)pyridin-3-yl]propanoic acid (3.3 g, 8.899 mmol, 1 equiv) and 2,6-lutidine (1.8 g, 18 mmol, 2 eq) in 30 mL of DCM was added with trimethylsilyl triflate (2.78 g, 13.5 mmol, 1.5 eq) dropwise at 0° C. over 5 min. The resulting mixture was stirred overnight at room temperature. The reaction mixture concentrated in vacuo and the residue was purified by reverse flash chromatography with the following conditions: column, C18 silica gel; mobile phase, Water in ACN, 0% to 100% gradient in 40 min; detector, UV 254 nm. This resulted in (2S)-2-amino-3-[5-chloro-2-(cyclopropylmethoxy)pyridin-3-yl]propanoic acid (1.6 g, 66.42%) as a white solid. LCMS: (ESI, m/z): [M+H]⁺=271

To a stirred solution of 1,4-dioxane:H₂O (40 mL, v/v=3/1) were added (2S)-2-amino-3-[5-chloro-2-(cyclopropylmethoxy)pyridin-3-yl]propanoic acid (1.5 g, 5.541 mmol, 1 equiv) and 2,5-dioxopyrrolidin-1-yl 9H-fluoren-9-ylmethyl carbonate (1.87 g, 5.541 mmol, 1 equiv) and Na₂CO₃ (2.33 g, 27.705 mmol, 5 equiv) at 0° C. under nitrogen atmosphere. The resulting mixture was stirred for additional 1-2 h at room temperature. The residue was acidified to pH=5 and then extracted with EtOAc (50 mL×3) and the organic layer was washed with brine, dried and concentrated in vacuo. The residue was purified by reverse flash chromatography with the following conditions: column, C18 silica gel; mobile phase, H2O in ACN, 0% to 100% gradient in 60 min; detector, UV 254 nm. to afford (2S)-3-[5-chloro-2-(cyclopropylmethoxy)pyridin-3-yl]-2-{[(9H-fluoren-9-ylmethoxy)carbonyl]amino}propanoic acid (691.5 mg, 25.32%) as a white solid. LCMS: (ESI, m/z): [M-tert-butyl]⁺=493

### Building Block 38: Preparation of (S)-2-((((9H-fluoren-9-yl)methoxy)carbonyl)amino)-3-(5-chloro-2-(pyridin-2-yl)phenyl)propanoic Acid

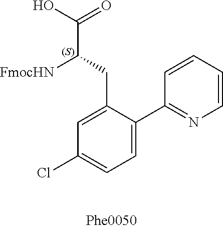

### Step 1: Synthesis of (2-bromo-5-chlorophenyl) methanol

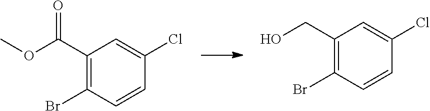

Into a solution of methyl 2-bromo-5-chlorobenzoate (10 g, 40.082 mmol, 1 equiv) in THF was added lithium aluminum hydride (1.0M in THF) (4.56 g, 120.246 mmol, 3 equiv) dropwise at 0° C. under nitrogen atmosphere. The resulting mixture was stirred for additional 2 h at room temperature. TLC showed ok (PE:EA=1:1). The reaction was quenched with sat. NH₄Cl (aq.) at 0° C. The resulting mixture was extracted with EtOAc (3×100 mL). The combined organic layers were dried over anhydrous Na2SO4. After filtration, the filtrate was concentrated under reduced pressure. The residue was purified by silica gel column chromatography, eluted with PE/EA (1:1) to afford (2-bromo-5-chlorophenyl) methanol (8 g, 90.12%) as a light brown oil. TLC: Rf=0.5 (PE/EA=1:1).

### Step 2: Synthesis of 1-bromo-2-(bromomethyl)-4-chlorobenzene

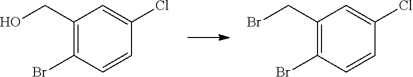 [SMILES: OCc1cc(Cl)ccc1Br ; Clc1ccc(Br)c(CBr)c1]

Into a solution of (2-bromo-5-chlorophenyl) methanol (8.0 g, 36.121 mmol, 1 equiv) in CH₂Cl₂ was added PBr₃ (19.55 g, 72.242 mmol, 2 equiv) dropwise at 0° C. under nitrogen atmosphere. The resulting mixture was stirred for additional 2 h at room temperature TLC was (PE/EA=1:1) shown the completion of the reaction. The reaction was quenched by the addition of sat. NH4Cl (aq.) (50 mL) at 0° C. The resulting mixture was extracted with EtOAc (3×70 mL). The combined organic layers were washed with brine (3×50 mL) and dried over anhydrous Na2SO4. After filtration, the filtrate was concentrated under reduced pressure. The residue was purified by silica gel column chromatography, eluted with PE/EA (1:1) to afford 1-bromo-2-(bromomethyl)-4-chlorobenzene (5.89 g, 57.34%) as a brown oil. TLC: Rf=0.5 (PE/EA=3:1)

### Step 3: Synthesis of tert-butyl (2S)-3-(2-bromo-5-chlorophenyl)-2-[(diphenylmethylidene)amino] propanoate

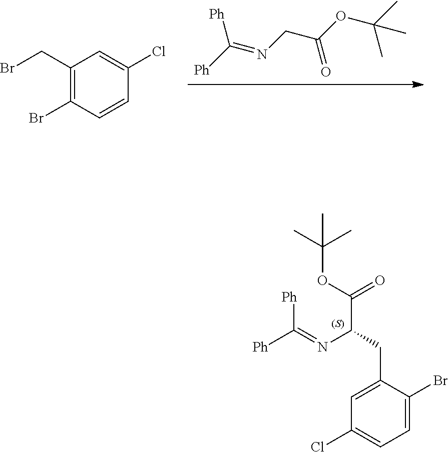

A solution of 1-bromo-2-(bromomethyl)-4-chlorobenzene (5.89 g, 20.712 mmol, 1 equiv) in CH₂Cl₂ (100 mL) was treated with tert-butyl 2-[(diphenylmethylidene)amino] acetate (6.12 g, 20.712 mmol, 1 equiv) and (2R,4R,5S)-1-(anthracen-9-ylmethyl)-5-ethenyl-2-[(S)-(prop-2-en-1-yloxy) (quinolin-4-yl) methyl]-1-azabicyclo [2.2.2] octan-1-ium bromide (0.63 g, 1.036 mmol, 0.05 equiv) for 30 min at 0° C. under nitrogen atmosphere followed by the addition of KOH (11.62 g, 207.120 mmol, 10 equiv) in water (100 mL) dropwise at 0° C. The resulting mixture was stirred for additional 2 h at 0° C. TLC detected product (PE/EA=4:1). The reaction was quenched by the addition of water (30 mL) at room temperature and extracted with ethyl acetate (3×100 mL). The combined organic layers were washed with brine (3×30 mL) and dried over anhydrous Na₂SO₄. After filtration, the filtrate was concentrated under reduced pressure. The residue was purified by silica gel column chromatography, eluted with PE/EA (4:1) to afford tert-butyl (2S)-3-(2-bromo-5-chlorophenyl)-2-[(diphenylmethylidene)amino] propanoate (1.5 g, 14.52%) as a light brown oil. LCMS: (ESI, m/z): [M+H]⁺=497.60

### Step 4: Synthesis of tert-butyl (2S)-3-[5-chloro-2-(pyridin-2-yl) phenyl]-2-[(diphenylmethylidene)amino] propanoate

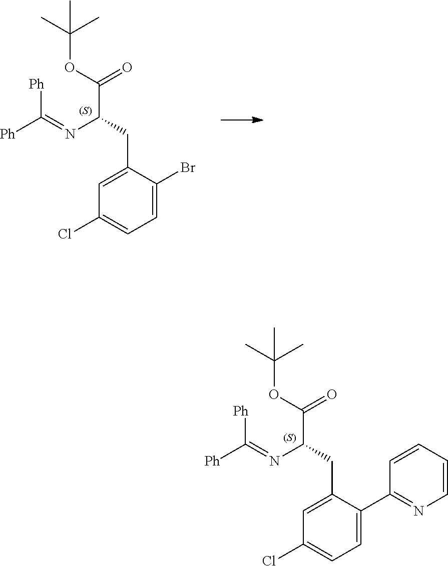

To a stirred mixture of tert-butyl (2S)-3-(2-bromo-5-chlorophenyl)-2-[(diphenylmethylidene)amino] propanoate (2.1 g, 4.210 mmol, 1 equiv) and 2-(tributylstannyl) pyridine (6.20 g, 16.840 mmol, 4 equiv) in 1,4-dioxane were added Pd(PPh₃)₄ (1.46 g, 1.263 mmol, 0.3 equiv) and CuI (0.80 g, 4.210 mmol, 1 equiv) in portions at room temperature under nitrogen atmosphere. The mixture was stirred for overnight at 80° C. Desired products could be detected by LCMS. The resulting mixture was added water (50 mL) and extracted with EtOAc (3×100 mL). The combined organic layers were washed with brine (2×50 mL) and dried over anhydrous Na₂SO₄. After filtration, the filtrate was concentrated under reduced pressure. The residue was purified by reverse flash chromatography with the following conditions: column, C18 silica gel; mobile phase, MeCN in water (0.1% FA), 0% to 100% gradient in 30 min; detector, UV 254 nm. This resulted in tert-butyl (2S)-3-[5-chloro-2-(pyridin-2-yl) phenyl]-2-[(diphenylmethylidene)amino] propanoate (1.5 g, 71.69%) as a light yellow oil. LCMS: (ESI, m/z): [M+H]⁺=497.20

### Step 5: Synthesis of (2S)-3-[5-chloro-2-(pyridin-2-yl) phenyl]-2-{[(9H-fluoren-9-ylmethoxy) carbonyl] amino} propanoic Acid

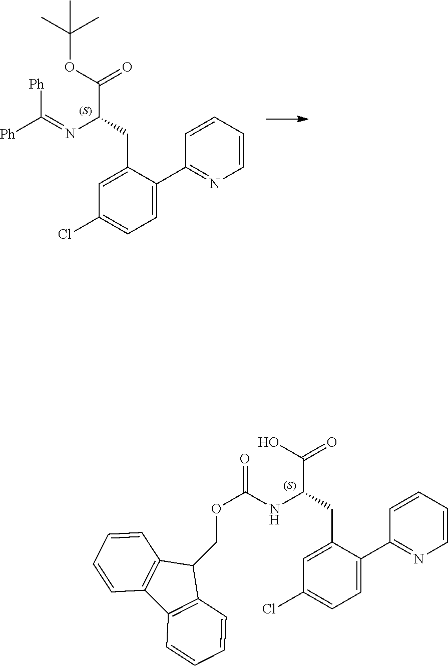

A solution of tert-butyl (2S)-3-[5-chloro-2-(pyridin-2-yl) phenyl]-2-[(diphenylmethylidene)amino] propanoate (1.5 g, 3.018 mmol, 1 equiv) in 1,4-dioxane (30 mL) was treated with HCl (6M) (20 mL, 658.256 mmol, 218.12 equiv) for 2 h at 50° C. under nitrogen atmosphere. Desired product could be detected by LCMS. The mixture was basified to pH 6 with NaOH (1M). The resulting mixture was used in the next step directly without further purification. LCMS: (ESI, m/z): [M+H]⁺=277.15.

Into a solution of (2S)-2-amino-3-[5-chloro-2-(pyridin-2-yl) phenyl] propanoic acid (1.2 g, 4.337 mmol, 1 equiv) and NaHCO₃ (0.52 g, 21.685 mmol, 5 equiv) in 1,4-dioxane (50 mL)/water (15 mL) was added 2,5-dioxopyrrolidin-1-yl 9H-fluoren-9-ylmethyl carbonate (1.61 g, 4.771 mmol, 1.1 equiv) in portions at room temperature under nitrogen atmosphere. Desired product could be detected by LCMS. The mixture was neutralized to pH=6 with CH₃COOH. The mixture was extracted with ethyl acetate (50 mL×2). The combined organic layers were concentrated in vacuo. The residue was purified by reverse flash chromatography with the following conditions: column, silica gel; mobile phase, MeCN in water, 10% to 100% gradient in 30 min; detector, UV 254 nm. This resulted in (2S)-3-[5-chloro-2-(pyridin-2-yl) phenyl]-2-{[(9H-fluoren-9-ylmethoxy) carbonyl] amino} propanoic acid (0.2511 g, 11.60%) as an off-white solid. LCMS: (ESI, m/z): [M+H]⁺=499.1

### Building Block 39: Preparation of (S)-2-((((9H-fluoren-9-yl)methoxy)carbonyl)amino)-3-(5-chloro-2-(1,3-dimethyl-1H-pyrazol-4-yl)phenyl)propanoic Acid

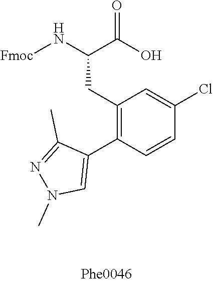

This compound was prepared following the general synthetic sequence described for the preparation of Building Block 38 using 1,3-dimethyl-4-(4,4,5,5-tetramethyl-1,3,2-dioxaborolan-2-yl)-1H-pyrazole in steps 4 to 5. LCMS: (ESI, m/z): [M+H]⁺=516.1.

### Building Block 40: Preparation of (S)-2-((((9Hfluoren-9-yl)methoxy)carbonyl)amino)-3-(5-chloro-2-(5-fluoropyridin-3-yl)phenyl)propanoic Acid

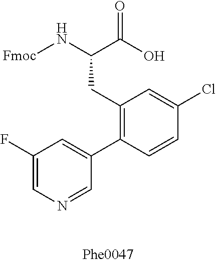

This compound was prepared following the general synthetic sequence described for the preparation of Building Block 38 using (5-fluoropyridin-3-yl)boronic acid in steps 4 to 5. LCMS: (ESI, m/z): [M+H]⁺=517.05.

### Building Block 41: Preparation of (S)-2-((((9H-fluoren-9-yl)methoxy)carbonyl)amino)-3-(5-chloro-2-(1-(difluoromethyl)-1H-pyrazol-4-yl)phenyl)propanoic Acid

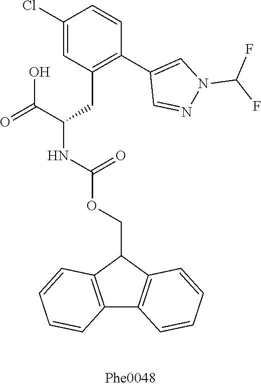 [SMILES: O=C(N[C@@H](Cc1cc(Cl)ccc1-c1cnn(C(F)F)c1)C(=O)O)OCC1c2ccccc2-c2ccccc21]

This compound was prepared following the general synthetic sequence described for the preparation of Building Block 38 using 1-(difluoromethyl)-4-(4,4,5,5-tetramethyl-1,3,2-dioxaborolan-2-yl)-1H-pyrazole in steps 4 to 5. LCMS: (ESI, m/z): [M+H]⁺=538.1.

### Building Block 42: Preparation of (S)-2-((((9H-fluoren-9-yl)methoxy)carbonyl)amino)-3-(5-chloro-2-(1-(trifluoromethyl)-1H-pyrazol-4-yl)phenyl)propanoic Acid

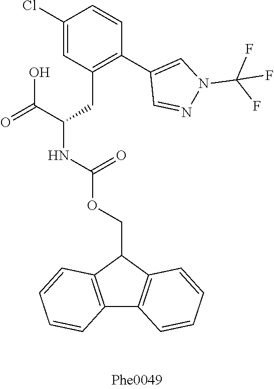 [SMILES: O=C(N[C@@H](Cc1cc(Cl)ccc1-c1cnn(C(F)(F)F)c1)C(=O)O)OCC1c2ccccc2-c2ccccc21]

This compound was prepared following the general synthetic sequence described for the preparation of Building Block 38 using 4-(4,4,5,5-tetramethyl-1,3,2-dioxaborolan-2-yl)-1-(trifluoromethyl)-1H-pyrazole in steps 4 to 5. LCMS: (ESI, m/z): [M+H]⁺=578.0.

### Building Block 43: Preparation of (S)-2-((((9H-fluoren-9-yl)methoxy)carbonyl)amino)-3-(5-chloro-2-(1,5-dimethyl-1H-pyrazol-4-yl)phenyl)propanoic Acid

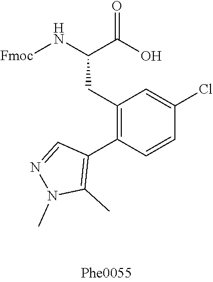

This compound was prepared following the general synthetic sequence described for the preparation of Building Block 38 using 1,5-dimethyl-4-(4,4,5,5-tetramethyl-1,3,2-dioxaborolan-2-yl)-1H-pyrazole in steps 4 to 5. LCMS: (ESI, m/z): [M+H]⁺=516.1.

### Building Block 44: Preparation of (S)-2-((((9H-fluoren-9-yl)methoxy)carbonyl)amino)-3-(5-chloro-2-(1,3-dimethyl-1H-pyrazol-5-yl)phenyl)propanoic Acid

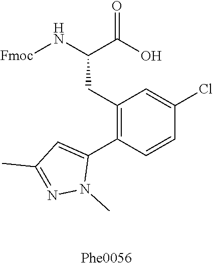

This compound was prepared following the general synthetic sequence described for the preparation of Building Block 38 using 1,3-dimethyl-5-(4,4,5,5-tetramethyl-1,3,2-dioxaborolan-2-yl)-1H-pyrazole in steps 4 to 5. LCMS: (ESI, m/z): [M+H]⁺=516.1.

### Building Block 45: Preparation of (S)-2-((((9H-fluoren-9-yl)methoxy)carbonyl)amino)-3-(5-chloro-2-(pyrimidin-5-yl)phenyl)propanoic Acid

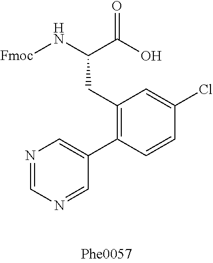

This compound was prepared following the general synthetic sequence described for the preparation of Building Block 38 using pyrimidin-5-ylboronic acid in steps 4 to 5. LCMS: (ESI, m/z): [M+H]⁺=500.1.

### Building Block 46: Preparation of (S)-2-((((9H-fluoren-9-yl)methoxy)carbonyl)amino)-3-(5-chloro-2-(morpholinomethyl)phenyl)propanoic Acid

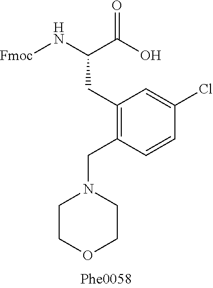

This compound was prepared following the general synthetic sequence described for the preparation of Building Block 38 using potassium trifluoro(morpholinomethyl)borate in steps 4 to 5. LCMS: (ESI, m/z): [M+H]⁺=521.09.

### Building Block 47: Preparation of (S)-2-((((9H-fluoren-9-yl)methoxy)carbonyl)amino)-3-(5-chloro-2-(thiazol-5-yl)phenyl)propanoic Acid

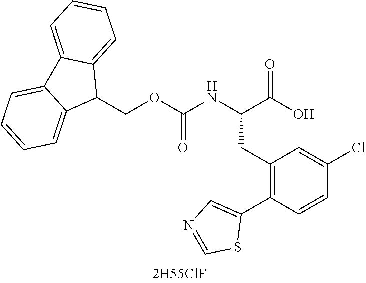 [SMILES: O=C(N[C@@H](Cc1cc(Cl)ccc1-c1cncs1)C(=O)O)OCC1c2ccccc2-c2ccccc21]

### Step 1: Synthesis of 5-chloro-2-(thiazol-5-yl)benzaldehyde

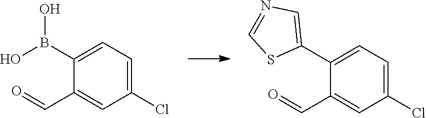 [SMILES: O=Cc1cc(Cl)ccc1B(O)O ; O=Cc1cc(Cl)ccc1-c1cncs1]

To a 100 ml round bottom flask was added 4-Chloro-2-formylphenylboronic acid (4.0 g, 21.73 mmol), 5-bromothiazole (3.0, 18.51 mmol) and [1,1′-Bis(diphenylphosphino)ferrocene]dichloropalladium(II), complex with dichloromethane (1.5 gr, 1.83 mmol). This was dissolved in dioxanes (90 ml) and 2M K2CO3 (22 ml). Nitrogen was bubbled in the mixture and the reaction was heated to 60° C. for 3 h. After completion the reaction was cooled and quenched with water. The crude was extracted with EtOAc 3× and the combined organics was dried over MgSO4, filtered, and solvent reduced. The crude was purified using column chromatography (15%) to afford 5-chloro-2-(thiazol-5-yl)benzaldehyde as a white solid (4.0 g, 98%) ESI MS m/z 222.1.

### Step 2: Synthesis of (5-chloro-2-(thiazol-5-yl)phenyl)methanol

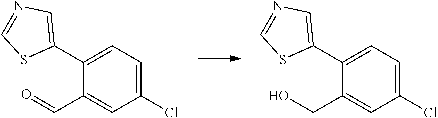 [SMILES: O=Cc1cc(Cl)ccc1-c1cncs1 ; OCc1cc(Cl)ccc1-c1cncs1]

Ethanol was added to 5-chloro-2-(thiazol-5-yl)benzaldehyde (4.04 g, 18.01 mmol) and the solution was cooled to 0° C. in an ice bath. Sodium borohydride (740 mg, 20 mmol) was added in 3 portions and the mixture was warmed to room temperature and allowed to react for 1 h. The solvent was reduced and 1N HCl was added The crude was then extracted with DCM 3×. The combined organics was dried over MgSO4, filtered, and solvent reduced. The crude was purified with column chromatography to afford the desired product, (5-chloro-2-(thiazol-5-yl)phenyl)methanol (4.0 gr, 98%), as a clear oil. ESI MS m/z 225.0

### Step 3: Synthesis of tert-butyl (S)-2-((((9H-fluoren-9-yl)methoxy)carbonyl)amino)-3-(5-chloro-2-(thiazol-5-yl)phenyl)propanoate

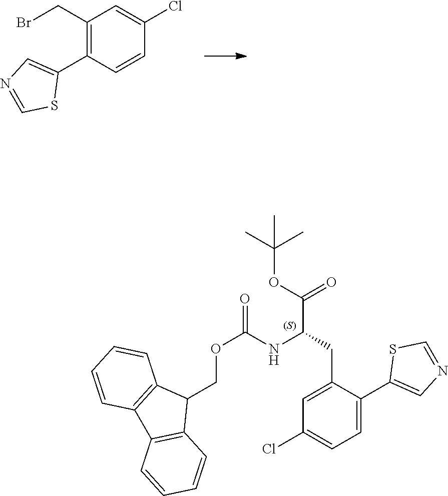

The starting material, (5-chloro-2-(thiazol-5-yl)phenyl)methanol (4.0 gr, 17.77 mmol), was dissolved in DCM (30 ml) and cooled to 0° C. in an ice bath. Dropwise, PBr3 (1.7 ml, 17.77 mmol) was added and the mixture was warmed to room temperature. This was allowed to react for 5 h. After completion the mixture was poured into a cold saturated solution of NaHCO₃. The crude product was extracted with DCM 3× and the combined organics was dried over MgSO4, filtered and solvent reduced. The crude product was taken onto the next step without further purification.

To a 100 ml round bottom flask was added O-Allyl-N-(9-anthracenylmethyl)cinchonidinium bromide (574 g, 0.94 mmol) and N-(Diphenylmethylene)glycine tert-butyl ester (2.83 g, 9.4 mmol). This was dissolved in DCM (60 ml) and the mixture was cooled to −20° C. To this was added 5-(2-(bromomethyl)-4-chlorophenyl)thiazole (3.3 g, 11.53 mmol) followed by 45% aqueous KOH (5.3 ml). The reaction was allowed to run for 16 hours at −20° C. Afterwards, water was added and the organics was extracted with DCM 3×. The combined organics was dried over MgSO4, filtered and reduced. The crude was then redissolved in dioxanes (100 ml). 2N HCl (20 ml) was added dropwise and the reaction was allowed to stir at room temperature for 1 h. Upon completion the mixture was cooled in an ice bath, and saturated NaHCO₃ was added until pH was 7-9. Then, a dissolved solution of FmocOSu (3.4 gr, 10.08 mmol) was added and the mixture was allowed to react for 12 h. The solution was quenched with water and organics extracted with EtOAc 3×. Combined organics was dried over MgSO4, filtered, and solvent reduced. The crude was purified over column chromatography (20% EtOAc/Hexanes) to afford the desired product, tert-butyl (S)-2-((((9H-fluoren-9-yl)methoxy)carbonyl)amino)-3-(5-chloro-2-(thiazol-5-yl)phenyl)propanoate as a clear oil (6.1 gr, 94%). ESI MS m/z 560.1.

### Step 4: Synthesis of tert-butyl (S)-2-((((9H-fluoren-9-yl)methoxy)carbonyl)amino)-3-(5-chloro-2-(thiazol-5-yl)phenyl)propanoate

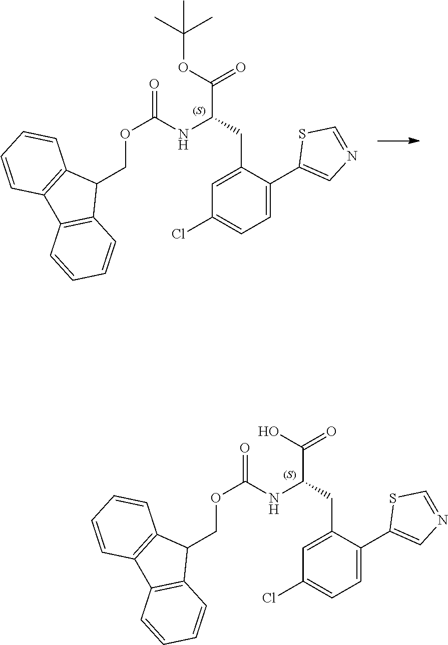

The starting material was dissolved in DCM (30 ml) and to this, a 50% TFA in DCM (30 ml) was added and the reaction was allowed to run at room temperature until complete. Afterwards, the solvent was reduced and the crude was purified over column chromatography (80% EtOAc/Hexanes) to afford the desired product tert-butyl (S)-2-((((9H-fluoren-9-yl)methoxy)carbonyl)amino)-3-(5-chloro-2-(thiazol-5-yl)phenyl)propanoate as a white solid. (5.0 gr, 91%) ESI MS m/z 504.9.

### Building Block 48: Preparation of (S)-2-((((9H-fluoren-9-yl)methoxy)carbonyl)amino)-3-(5-chloro-2-(thiazol-2-yl)phenyl)propanoic Acid

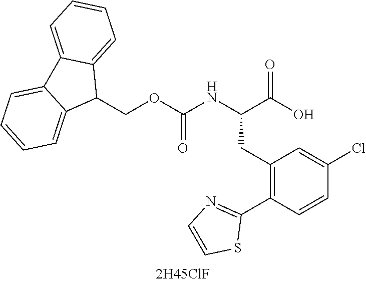 [SMILES: O=C(N[C@@H](Cc1cc(Cl)ccc1-c1nccs1)C(=O)O)OCC1c2ccccc2-c2ccccc21]

This compound was prepared following the general synthetic sequence described for the preparation of Building Block 47 using 2-bromothiazole in steps 1 to 4 instead of 5-bromothiazole. ESI MS m/z 504.09

### Building Block 49: Preparation of (S)-2-((((9H-fluoren-9-yl)methoxy)carbonyl)amino)-3-(5-chloro-2-(1-methyl-1H-pyrazol-3-yl)phenyl)propanoic Acid

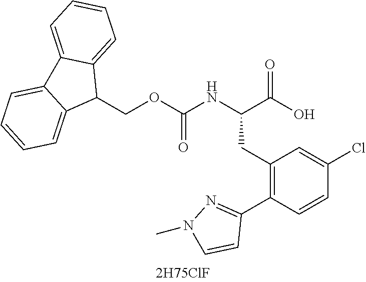 [SMILES: Cn1ccc(-c2ccc(Cl)cc2C[C@H](NC(=O)OCC2c3ccccc3-c3ccccc32)C(=O)O)n1]

This compound was prepared following the general synthetic sequence described for the preparation of Building Block 47 using 3-bromopyrazole in steps 1 to 4 instead of 5-bromothiazole. ESI MS m/z 501.15.

### Building Block 50: Preparation of (S)-2-((((9H-fluoren-9-yl)methoxy)carbonyl)amino)-3-(5-chloro-2-(1-methyl-1H-pyrazol-4-yl)phenyl)propanoic Acid

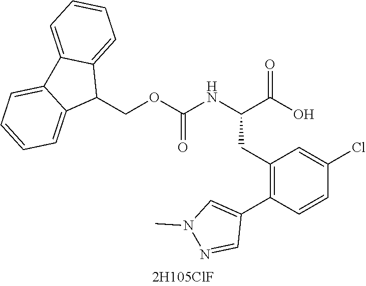 [SMILES: Cn1cc(-c2ccc(Cl)cc2C[C@H](NC(=O)OCC2c3ccccc3-c3ccccc32)C(=O)O)cn1]

This compound was prepared following the general synthetic sequence described for the preparation of Building Block 47 using 4-bromopyrazole in steps 1 to 4 instead of 5-bromothiazole. ESI MS m/z 501.15.

### Building Block 51: Preparation of (S)-2-((((9H-fluoren-9-yl)methoxy)carbonyl)amino)-3-(5-fluoro-2-(1-methyl-1H-pyrazol-4-yl)phenyl)propanoic Acid

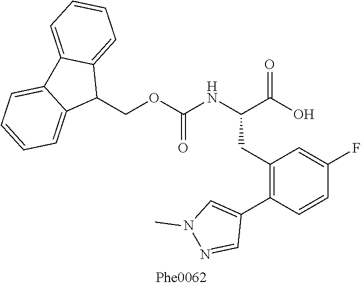 [SMILES: Cn1cc(-c2ccc(F)cc2C[C@H](NC(=O)OCC2c3ccccc3-c3ccccc32)C(=O)O)cn1]

This compound was prepared following the general synthetic sequence described for the preparation of Building Block 47 using 4-fluoro-2-formylphenylboronic acid and 3-bromopyrazole in steps 1 to 4. ESI MS m/z 485.18.

### Building Block 52: Preparation of (S)-2-((((9H-fluoren-9-yl)methoxy)carbonyl)amino)-3-(5-chloro-2-(thiazol-4-yl)phenyl)propanoic Acid

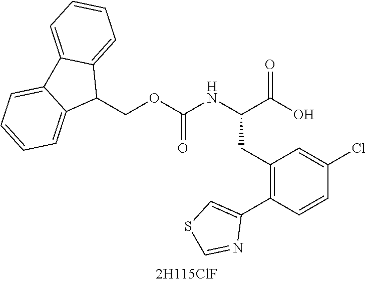 [SMILES: O=C(N[C@@H](Cc1cc(Cl)ccc1-c1cscn1)C(=O)O)OCC1c2ccccc2-c2ccccc21]

This compound was prepared following the general synthetic sequence described for the preparation of Building Block 47 using 4-bromothiazole in steps 1 to 4 instead of 5-bromothiazole. ESI MS m/z 504.09.

### Building Block 53: Preparation of (S)-2-((((9H-fluoren-9-yl)methoxy)carbonyl)amino)-3-(5-chloro-2-(4-methylthiazol-5-yl)phenyl)propanoic Acid

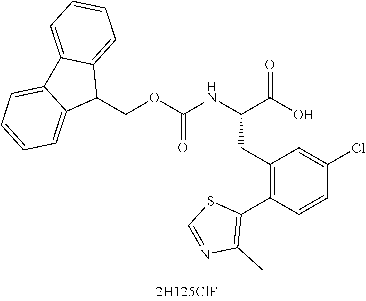 [SMILES: Cc1ncsc1-c1ccc(Cl)cc1C[C@H](NC(=O)OCC1c2ccccc2-c2ccccc21)C(=O)O]

This compound was prepared following the general synthetic sequence described for the preparation of Building Block 47 using 5-bromo-4-methylthiazole in steps 1 to 4 instead of 5-bromothiazole. ESI MS m/z 518.11.

### Building Block 54: Preparation of (S)-2-((((9H-fluoren-9-yl)methoxy)carbonyl)amino)-3-(5-chloro-2-(2,4-dimethylthiazol-5-yl)phenyl)propanoic Acid

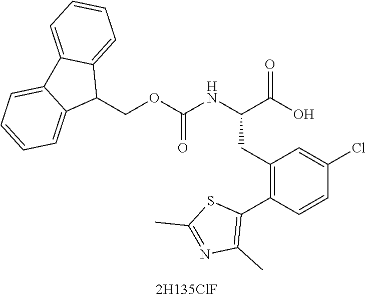 [SMILES: Cc1nc(C)c(-c2ccc(Cl)cc2C[C@H](NC(=O)OCC2c3ccccc3-c3ccccc32)C(=O)O)s1]

This compound was prepared following the general synthetic sequence described for the preparation of Building Block 47 using 5-bromo-2-methylthiazole in steps 1 to 4 instead of 5-bromothiazole ESI MS m/z 532.12.

### Building Block 55: Preparation of (S)-2-((((9H-fluoren-9-yl)methoxy)carbonyl)amino)-3-(5-chloro-2-(1,3,4-thiadiazol-2-yl)phenyl)propanoic Acid

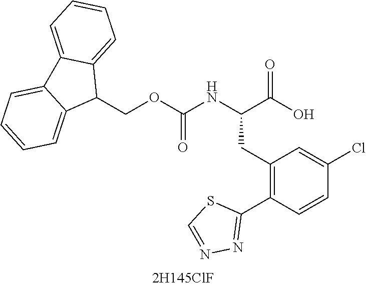 [SMILES: O=C(N[C@@H](Cc1cc(Cl)ccc1-c1nncs1)C(=O)O)OCC1c2ccccc2-c2ccccc21]

This compound was prepared following the general synthetic sequence described for the preparation of Building Block 47 using 2-bromo-1,3,4-thiadiazole in steps 1 to 4 instead of 5-bromothiazole. ESI MS m/z 505.09.

### Building Block 56: Preparation of (S)-2-((((9H-fluoren-9-yl)methoxy)carbonyl)amino)-3-(5-chloro-2-(2-methyl-2H-1,2,3-triazol-4-yl)phenyl)propanoic Acid

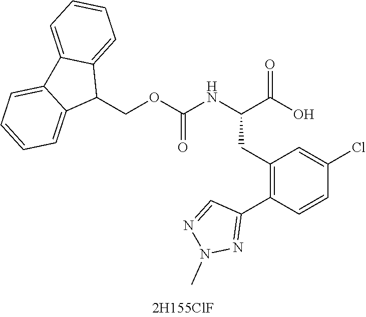 [SMILES: Cn1ncc(-c2ccc(Cl)cc2C[C@H](NC(=O)OCC2c3ccccc3-c3ccccc32)C(=O)O)n1]

This compound was prepared following the general synthetic sequence described for the preparation of Building Block 47 using 4-bromo-2-methyl-2H-1,2,3-triazole in steps 1 to 4 instead of 5-bromothiazole. ESI MS m/z 502.14.

### Building Block 57: Preparation of (S)-2-((((9H-fluoren-9-yl)methoxy)carbonyl)amino)-3-(5-chloro-2-(2-methylthiazol-5-yl)phenyl)propanoic Acid

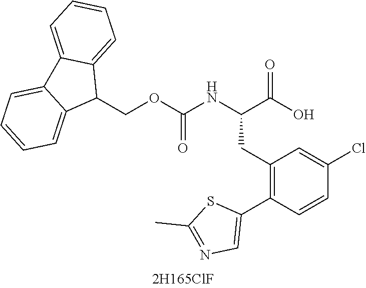 [SMILES: Cc1ncc(-c2ccc(Cl)cc2C[C@H](NC(=O)OCC2c3ccccc3-c3ccccc32)C(=O)O)s1]

This compound was prepared following the general synthetic sequence described for the preparation of Building Block 47. ESI MS m/z 518.11.

### Building Block 58: Preparation of (S)-2((((9H-fluoren-9-yl)methoxy)carbonyl)amino)-3-(5-fluoro-2-(thiazole-5-yl)phenyl propanoic Acid

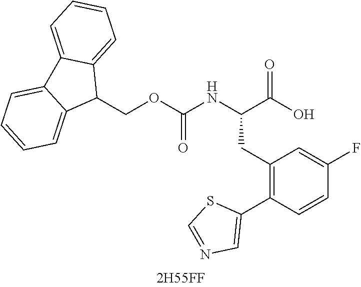 [SMILES: O=C(N[C@@H](Cc1cc(F)ccc1-c1cncs1)C(=O)O)OCC1c2ccccc2-c2ccccc21]

This compound was prepared following the general synthetic sequence described for the preparation of Building Block 47 using 5-bromothiazole and (4-fluoro-2-formylphenyl)boronic acid in steps 1 to 4 instead of 5-bromothiazole and (4-chloro-2-formylphenyl)boronic acid. ESI MS m/z 488.12.

### Building Block 59: Preparation of (S)-2-((((9H-fluoren-9-yl)methoxy)carbonyl)amino)-3-(5-chloro-2-(5-methyl-1,3,4-thiadiazol-2-yl)phenyl)propanoic Acid

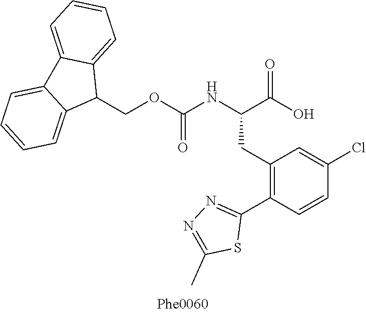 [SMILES: Cc1nnc(-c2ccc(Cl)cc2C[C@H](NC(=O)OCC2c3ccccc3-c3ccccc32)C(=O)O)s1]

This compound was prepared following the general synthetic sequence described for the preparation of Building Block 47 using 2-bromo-5-methyl-1,3,4-thiadiazole in steps 1 to 4 instead of 5-bromothiazole. ESI MS m/z 519.10.

### Building Block 60: Preparation of (S)-2-((((9H-fluoren-9-yl)methoxy)carbonyl)amino)-3-(5-chloro-2-(1-methyl-1H-pyrazol-5-yl)phenyl)propanoic Acid

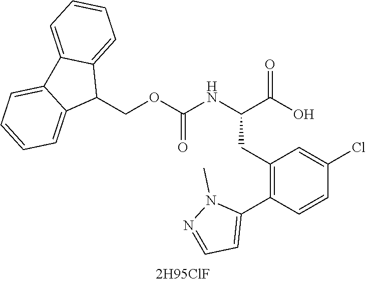 [SMILES: Cn1nccc1-c1ccc(Cl)cc1C[C@H](NC(=O)OCC1c2ccccc2-c2ccccc21)C(=O)O]

This compound was prepared following the general synthetic sequence described for the preparation of Building Block 47 using 5-bromo-1-methyl-1H-pyrazole in steps 1 to 4 instead of 5-bromothiazole. ESI MS m/z 501.15.

### Building Block 61: Preparation of (S)-2-((((9H-fluoren-9-yl)methoxy)carbonyl)amino)-3-(5-chloro-2-(pyridin-3-yl)phenyl)propanoic Acid

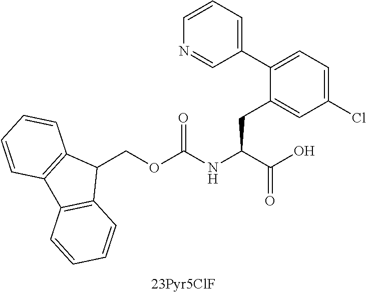 [SMILES: O=C(N[C@@H](Cc1cc(Cl)ccc1-c1cccnc1)C(=O)O)OCC1c2ccccc2-c2ccccc21]

This compound was prepared following the general synthetic sequence described for the preparation of Building Block 47 using 3-bromopyridine in steps 1 to 4 instead of 5-bromothiazole. ESI MS m/z 498.1.

### Building Block 62: Preparation of (S)-2-((((9H-fluoren-9-yl)methoxy)carbonyl)amino)-3-(4-chloro-[1,1′-biphenyl]-2-yl)propanoic Acid

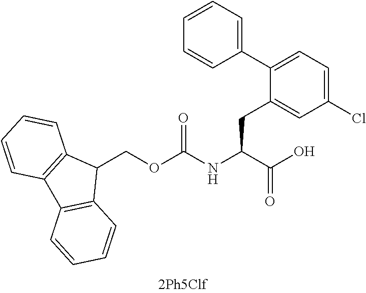 [SMILES: O=C(N[C@@H](Cc1cc(Cl)ccc1-c1ccccc1)C(=O)O)OCC1c2ccccc2-c2ccccc21]

This compound was prepared following the general synthetic sequence described for the preparation of Building Block 38 using bromobenzene in steps 1 to 4 instead of 5-bromothiazole. LCMS: (ESI, m/z): [M+H]⁺=497.14.

### Building Block 63: Preparation of (S)-2-((((9H-fluoren-9-yl)methoxy)carbonyl)amino)-3-(5-chloro-2-(1H-1,2,3-triazol-1-yl)phenyl)propanoic Acid

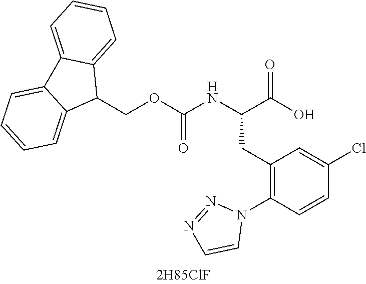 [SMILES: O=C(N[C@@H](Cc1cc(Cl)ccc1-n1ccnn1)C(=O)O)OCC1c2ccccc2-c2ccccc21]

5-chloro-2-fluorobenzaldehyde (2.0 g, 12.7 mmol) and sodium azide (852 mg, 13.10 mmol) was dissolved in DMF (6 ml). This mixture was heated to 60° C. and allowed to react for 8 h then cooled to room temperature. The reaction mixture was diluted with water and DCM which was then acidified with 1N HCl until the pH read 4. The organics was extracted with DCM 3×, dried over MgSO4, filtered, and solvent reduced. The crude mixture was purified over column chromatography (15% EtOAc/Hex) to afford the desired product (1.0 g, 86%). ESI MS m/z: 181.0.

To a round bottom flask was combined 2-azido-5-chlorobenzaldehyde (1 g, 5.52 mmol), trimethylsilylacetylene (852 ul, 5.79 mmol), CuSO4 (137 mg, 0.55 mmol) and sodium ascorbate (220 mg, 1.11 mmol). This was dissolved in a 4:1 mixture of t-butanol (20 ml) and water (5 ml). This reaction was allowed to react at 50° C. for 12 h then cooled to room temperature. The mixture was washed with water and organics was extracted with DCM 3×. The combined organics was dried over MgSO4, filtered, and reduced. The crude product was purified over column chromatography (25% EtOAc/hexanes) to afford the desired product (600 mg, 54%). ESI MS m/z: 207.02

Dissolved 5-chloro-2-(1H-1,2,3-triazol-1-yl)benzaldehyde (600 mg, 2.89 mmol) in methanol and cooled to 0° C. in an ice bath. Sodium borohydride (130 mg, 3.51 mmol) was added in two portions. The compound was warmed to room temperature and allowed to react for 1 h. The solvent was reduced and 1N HCl was added The crude was extracted with DCM 3×. The combined organics was dried over MgSO4, filtered, and solvent reduced. The crude was purified with column chromatography to afford the desired product, (5-chloro-2-(1H-1,2,3-triazol-1-yl)phenyl)methanol as a clear oil (600 mg, 99%). ESI MS m/z: 225.0.

Dissolved (5-chloro-2-(1H-1,2,3-triazol-1-yl)phenyl)methanol (600 mg, 2.89 mmol) in DCM (20 ml) and cooled to 0° C. in an ice bath. Dropwise, added in phosphorus tribromide (390 ul, 2.89 mmol) and the mixture was warmed to room temperature and allowed to react for 12 h. The reaction was then transferred into an ice cold solution of saturated NaHCO3 until basic. Then the organics was extracted with DCM 3×. The combined organics was dried over MgSO4, filtered, and reduced. The crude material was taken onto the next reaction without further purification.

To a 100 ml round bottom flask was added O-Allyl-N-(9-anthracenylmethyl)cinchonidinium bromide (40 mg, 0.06 mmol) and N-(Diphenylmethylene)glycine tert-butyl ester (180 mg, 0.61 mmol). This was dissolved in DCM (15 ml) and the mixture was cooled to −20° C. To this was added 1-(2-(bromomethyl)-4-chlorophenyl)-1H-1,2,3-triazole (200 mg, 0.074 mmol) followed by 45% aqueous KOH (340 ul). The reaction was allowed to run for 16 hours at −20° C. Afterwards, water was added and the organics was extracted with DCM 3×. The combined organics was dried over MgSO4, filtered and reduced. The crude was then redissolved in dioxanes. 2N HCl (3 ml) was added dropwise and the reaction was allowed to stir at room temperature for 1 h. Upon completion the mixture was cooled in an ice bath, and saturated NaHCO3 was added until pH was 7-9. Then, a dissolved solution of FmocOSu (215 mg, 6.37 mmol) was added and the mixture and allowed to react for 12 h. The solution was quenched with water and organics extracted with EtOAc 3×. Combined organics was dried over MgSO4, filtered, and solvent reduced. The crude was purified over column chromatography to afford the desired product, tert-butyl (S)-2-((((9H-fluoren-9-yl)methoxy)carbonyl)amino)-3-(5-chloro-2-(1H-1,2,3-triazol-1-yl)phenyl)propanoate as a clear oil (350 mg, 86%). ESI MS m/z 544.19.

The starting material was dissolved in DCM (10 ml) and to this, a 50% TFA in DCM (10 ml) was added and the reaction was allowed to run at room temperature until complete. Afterwards, the solvent was reduced and the crude was purified over column chromatography (60% EtOAc/Hexanes) to (S)-2-((((9H-fluoren-9-yl)methoxy)carbonyl)amino)-3-(5-chloro-2-(1H-1,2,3-triazol-1-yl)phenyl)propanoic acid as a white solid (300 mg, 95%) ESI MS m/z 488.13.

### Building Block 64: Preparation of (S)-2-((((9H-fluoren-9-yl)methoxy)carbonyl)amino)-3-(5-chloro-2-(dimethylamino)phenyl)propanoic Acid

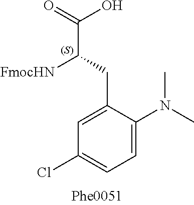

Into a 250 ml round bottom was placed 4-chloro-2-iodoaniline (5 g, 19.726 mmol, 1 equiv) in DMF (20 ml), NaH (2.37 g, 98.630 mmol, 5.00 equiv) was added at 0° C. under nitrogen atmosphere. The mixture was stirred at 0° C. for 30 min. CH₃I (14.00 g, 98.630 mmol, 5 equiv) was added dropwise over 10 min at 0° C. The resulting mixture was stirred at room temperature for 16 h. The reaction was quenched with ice-water (500 mL) and extracted with EtOAc (3×200 mL). The organic layer combined and washed with brine (100 mL), dried over anhydrous Na₂SO₄. After filtration, the filtrate was concentrated under reduced pressure. The residue was purified by silica gel column chromatography, eluted with PE/EA (0-50%). This resulted in 4-chloro-2-iodo-N,N-dimethylaniline (4 g, 72.03%) as a yellow oil. LCMS: (ESI, m/z): [M+H]⁺=281.85.

To a mixture of Zn (1.6 g, 24.14 mmol, 1.7 equiv) in DMA (10 mL) was added ethylene dibromide (374 mg, 2 mmol, 0.14 equiv) in one portion under N₂. Then TMSCl (153.4 mg, 1.42 mmol, 0.1 equiv) was added slowly and the mixture was stirred for 30 min at 25° C. A solution of (R)-methyl 2-(tert-butoxycarbonylamino)-3-iodopropanoate (7 g, 21.3 mmol, 1.5 equiv) in DMA (10 mL) was added dropwise slowly (30 min) to maintain temperature below 50° C., the resulting mixture was stirred at rt for 2 h and then added via a cannula to a solution of 4-chloro-2-iodo-N,N-dimethylaniline (4 g, 14.2 mmol, 1 equiv), Pd(dppf)Cl₂·CH₂Cl₂ (2.31 g, 2.842 mmol, 0.2 equiv) and CuI (0.54 g, 2.842 mmol, 0.2 equiv) in DMA (20 ml) under N₂, the color of the mixture turned brown, then the mixture was heated and stirred at 80° C. for 2 h under N₂. The mixture was quenched with ice-water (200 ml) and extracted with EtOAc (3×50 ml). The organic layer was combined and washed with brine (100 ml), dried with anhydrous Na₂SO₄, filtered and concentrated in vacuum to give the crude product. The crude product was purified

Into a 100 ml round bottom was placed methyl (2S)-2-[(tert-butoxycarbonyl)amino]-3-[5-chloro-2-(dimethylamino)phenyl]propanoate (1.88 g, 5.268 mmol, 1 equiv) in THF (20 mL). NaOH (1.05 g, 26.340 mmol, 5 equiv) in H₂O (4 mL) was added at 0° C. under air atmosphere. The resulting mixture was stirred for 2 h at room temperature. The mixture was acidified to PH=6 with 2N HCl (aq.). The resulting mixture was extracted with EtOAc (2×200 mL). The organic layer combined and washed with brine, dried over with anhydrous Na₂SO₄. After filtration, the filtrate was concentrated under reduced pressure. The residue was purified by silica gel column chromatography, eluted with PE/EA (0-50%) to afford (2S)-2-[(tert-butoxycarbonyl)amino]-3-[5-chloro-2-(dimethylamino)phenyl]propanoic acid (1.8 g, 99.66%) as a white solid. LCMS: (ESI, m/z): [M+H]+=343.05.

Into a 100 ml round bottom was placed (2S)-2-[(tert-butoxycarbonyl)amino]-3-[5-chloro-2-(dimethylamino)phenyl]propanoic acid (1.8 g, 5.251 mmol, 1 equiv) in DCM, TFA (20 mL, 269.261 mmol, 51.28 equiv) was added at room

temperature under nitrogen atmosphere. The resulting mixture was stirred for 2 h at room temperature. The solvent was removed by under reduced pressure. The crude product was used in the next step directly without further purification. LCMS: (ESI, m/z): [M+H]+=243.05.

Into a 100 ml round bottom was placed (2S)-2-amino-3-[5-chloro-2-(dimethylamino)phenyl]propanoic acid (1.8 g, 7.417 mmol, 1 equiv) in 1,4-dioxane (30 mL) and H₂O (10 mL), NaHCO₃ (3.13 g, 37.1910 mmol, 5 equiv) and 2,5-dioxopyrrolidin-1-yl 9H-fluoren-9-ylmethyl carbonate (2.01 g, 5.9526 mmol, 0.8 equiv) was added at room temperature under air atmosphere. The resulting mixture was stirred at room temperature for 16 h. The mixture was acidified to PH=6 with 2N HCl (aq) and extracted with EtOAc (3×100 mL). The organic layer was combined and dried over anhydrous Na₂SO₄. After filtration, the filtrate was concentrated under reduced pressure. The residue was purified by reverse flash chromatography with the following conditions: column, silica gel; mobile phase, MeCN in water, 10% to 50% gradient in 10 min; detector, UV 254 nm. This resulted in (2S)-3-[5-chloro-2-(dimethylamino)phenyl]-2-{[(9H-fluoren-9-ylmethoxy)carbonyl]amino}propanoic acid (708.6 mg, 20.55%) as a white solid. LCMS: (ESI, m/z): [M+H]⁺=464.15.

### Building Block 65: Preparation of (S)-2-((((9H-fluoren-9-yl)methoxy)carbonyl)amino)-3-(5-chloro-2-(methoxymethyl)phenyl)propanoic Acid

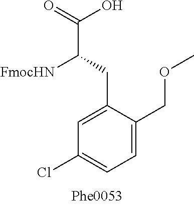

To a stirred solution of (4-chloro-2-iodophenyl) methanol (3 g, 11.174 mmol, 1 equiv) in DMF (60 mL) was added NaH (0.80 g, 33.522 mmol, 3 equiv) in portions at 0° C. under air atmosphere. The resulting mixture was stirred for 30 min at room temperature under air atmosphere. CH₃I (7.93 g, 55.870 mmol, 5 equiv) was added to the solution and stirred for 16 h at room temperature. The reaction was quenched with sat. NH₄Cl (aq.) at 0° C. The resulting mixture was extracted with EtOAc (3×100 mL). The combined organic layers were washed with brine (1×50 mL), dried over anhydrous Na₂SO₄ and filtered. The filtrate was concentrated under vacuum. The residue was purified by reverse flash chromatography with the following conditions: column, C18 silica gel; mobile phase, Acetonitrile in Water, 0% to 100% gradient in 40 min; detector, UV 254 nm. Pure fractions were evaporated to dryness to afford 4-chloro-2-iodo-1-(methoxymethyl) benzene (3.3 g, 104.54%) as a light yellow oil.

A solution of Zn (2.11 g, 32.282 mmol, 2.4 equiv) in DMA (20 mL) was added 1,2-Dibromoethane (0.26 g, 1.345 mmol, 0.1 equiv) in one portion under nitrogen. Then TMSCl (97.91 mg, 0.901 mmol, 0.067 equiv) was added dropwise at 20° C. and stirred for 30 min at room temperature. Methyl (2R)-2-[(tert-butoxycarbonyl) amino]-3-iodopropanoate (8.85 g, 26.902 mmol, 2 equiv) in DMA (20 mL) was added to the mixture, the temperature risen up to 50° C. and stirred for 1.5 h at room temperature under nitrogen atmosphere. The above mixture was added to a solution of 4-chloro-2-iodo-1-(methoxymethyl) benzene (3.8 g, 13.451 mmol, 1 equiv), CuI (0.51 g, 2.690 mmol, 0.2 equiv), Pd(dppf)Cl₂ (0.98 g, 1.345 mmol, 0.1 equiv) in DMA (30 mL). The resulting mixture was stirred for 2 h at 80° C. under nitrogen atmosphere. Desired product could be detected by LCMS. The reaction was quenched with sat. NH₄Cl (aq.) at 0° C. and extracted with EtOAc (3×200 mL). The combined organic layers were washed with brine (3×100 mL) and dried over anhydrous Na₂SO₄. After filtration, the filtrate was concentrated under vacuum. The residue was purified by reverse flash chromatography with the following conditions: column, C18 silica gel; mobile phase, Acetonitrile in Water (0.1% FA), 0% to 100% gradient in 40 min; detector, UV 254 nm. Pure fractions were evaporated to dryness to afford methyl (2S)-2-[(tert-butoxycarbonyl) amino]-3-[5-chloro-2-(methoxymethyl) phenyl]propanoate (3.8 g, 78.95%) as a light brown solid. LCMS: (ESI, m/z): [M+H]⁺=380.15.

A solution of methyl (2S)-2-[(tert-butoxycarbonyl) amino]-3-[5-chloro-2-(methoxymethyl) phenyl] propanoate (200 mg, 0.559 mmol, 1 equiv) and LiOH (0.67 g, 27.945 mmol, 5 equiv) in THF (30 mL)/H₂O (10 mL) was stirred for 2 h at room temperature. Desired product could be detected by LCMS. The mixture was acidified to pH 5 with HCl (1N) and extracted with EtOAc (3×20 mL). The combined organic layers were washed with brine (1×10 mL) and dried over anhydrous Na₂SO₄. After filtration, the filtrate was concentrated under vacuum to afford (2S)-2-[(tert-butoxycarbonyl) amino]-3-[5-chloro-2-(methoxymethyl) phenyl] propanoic acid (1.9 g, 98.88%) as a yellow oil. LCMS: (ESI, m/z): [M+H]⁺=366.10.

A solution of (2S)-2-[(tert-butoxycarbonyl) amino]-3-[5-chloro-2-(methoxymethyl)phenyl]propanoic acid (1.8 g, 5.236 mmol, 1 equiv) in HCl(gas) in 1,4-dioxane (40 mL) was stirred for 2 h at room temperature. Desired product could be detected by LCMS. The resulting mixture was concentrated under vacuum to afford crude product (2S)-2-amino-3-[5-chloro-2-(methoxymethyl) phenyl] propanoic acid (1.2 g, 94.05%) as a light brown oil which was used for next step without further purification. LCMS: (ESI, m/z): [M+H]⁺=244.10

Into a solution of (2S)-2-amino-3-[5-chloro-2-(methoxymethyl) phenyl] propanoic acid (1.5 g, 6.155 mmol, 1 equiv) in THF (30 mL, 370.283 mmol)/H₂O (10 mL, 555.093 mmol) was NaHCO₃ (3.88 g, 46.163 mmol, 7.5 equiv) and 2,5-dioxopyrrolidin-1-yl 9H-fluoren-9-ylmethyl carbonate (2.28 g, 6.771 mmol, 1.1 equiv). The resulting solution was stirred for 16 h at room temperature. Desired product could be detected by LCMS. The mixture was acidified to pH 5 with HCl (1N) and extracted with EtOAc (3×150 mL). The combined organic layers were washed with brine (1×100 mL) and dried over anhydrous Na₂SO₄. After filtration, the filtrate was concentrated under vacuum. The crude product (1.8 g) was purified by Prep-HPLC with the following conditions: Column: XBridge BEH C18 OBD Prep Column, 19*250 mm, 5 μm; Mobile Phase A: Water (0.05% FA), Mobile Phase B: ACN; Flow rate: 80 mL/min; Gradient: 59% B to 59% B in 22 min; Wave Length: 220 nm; RT1(min): 16.5; Number of Runs: 0). This resulted in (2S)-3-[5-chloro-2-(methoxymethyl) phenyl]-2-{[(9H-fluoren-9-ylmethoxy) carbonyl] amino} propanoic acid (1.8025 g, 62.76%) as a white solid. LCMS: (ESI, m/z): [M+H]⁺=488.1.

### Building Block 66: Preparation of (S)-2-((((9H-fluoren-9-yl)methoxy)carbonyl)amino)-6-oxo-6-(piperidin-1-yl)hexanoic Acid

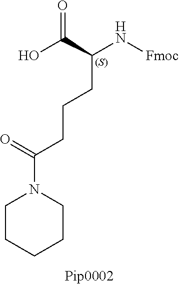

To a stirred solution of aminoadipate (20 g, 124.103 mmol, 1.00 equiv) in dioxane (1 L) was added sodium dicarbonate (52.13 g, 620.515 mmol, 5 equiv) in H₂O (300 mL). To the above mixture was added 2,5-dioxopyrrolidin-1-yl 9H-fluoren-9-ylmethyl carbonate (50.24 g, 148.924 mmol, 1.2 equiv) at 0° C. The resulting mixture was stirred for additional over night at room temperature. The reaction was monitored by LCMS. The mixture was allowed to cool down to −5 degrees C. and acidified to pH 1˜2 with dilute HCl. The aqueous layer was extracted with ethyl acetate (3×200 mL). The organics was dried over Na₂SO₄ and concentrated under vacuum. The residue was purified by silica gel column chromatography, eluted with EA:PE (1:1) to afford (2S)-2-{[(9H-fluoren-9-ylmethoxy)carbonyl]amino}hexanedioic acid (35 g, 73.56%) as a white solid. LCMS: (ESI, m/z): [M+Na]⁺=406.

A solution/mixture of (2S)-2-{[(9H-fluoren-9-ylmethoxy)carbonyl]amino}hexanedioic acid (5 g, 13.041 mmol, 1.00 equiv), polyoxymethylene (7.5 g, 6.5 equiv) and para-toluene sulfonate (0.22 g, 1.304 mmol, 0.1 equiv) in toluene (300 mL) was stirred for 16 h at 120° C. under nitrogen atmosphere. The mixture was allowed to cool down to the room temperature. The resulting mixture was filtered. The filter cake was washed with ethyl acetate (100 mL). The combined filtrates were concentrated under reduced pressure. The residue was purified by reverse flash chromatography to afford to 4-[(4S)-3-[(9H-fluoren-9-ylmethoxy)carbonyl]-5-oxo-1,3-oxazolidin-4-yl]butanoic acid (5.1 g) as a white solid. LCMS: (ESI, m/z): [M+H]⁺=396.41.

A solution of 4-[(4S)-3-[(9H-fluoren-9-ylmethoxy)carbonyl]-5-oxo-1,3-oxazolidin-4-yl]butanoic acid (6.007 g, 12.153 mmol, 1.00 equiv), piperidine (1.03 g, 12.153 mmol, 1.0 equiv), [chloro(dimethylamino)methylidene]dimethylazanium; hexafluoro-l\hat{ }[5]-phosphanuide (5.11 g, 18.230 mmol, 1.5 equiv) and 1-methyl-1H-imidazole (2.99 g, 36.459 mmol, 3.0 equiv) in CH₃CN (300 mL, 49.94 equiv) was stirred for overnight at 50° C. under nitrogen atmosphere. The resulting mixture was extracted with ethyl acetate (3×100 mL). The combined organic layers were washed with saturated NaCl (3×100 mL), dried over anhydrous Na₂SO₄ and concentrated under reduced pressure. The residue was purified by reverse phase flash with the following conditions (EA:PE, 1:3) to afford 9H-fluoren-9-ylmethyl (4S)-5-oxo-4-[4-oxo-4-(piperidin-1-yl)butyl]-1,3-oxazolidine-3-carboxylate (4.7 g, 83.61%) as a colorless semi-solid. LCMS: (ESI, m/z): [M+H]⁺=463.

To a stirred solution of 9H-fluoren-9-ylmethyl (4S)-5-oxo-4-[4-oxo-4-(piperidin-1-yl)butyl]-1,3-oxazolidine-3-carboxylate (5.46 g, 11.804 mmol, 1.00 equiv) in THF (100 mL) were added NaOH (1.89 g, 47.216 mmol, 4.0 equiv) and H₂O (47 mL) at 0° C. under nitrogen atmosphere. The mixture was allowed to warm to room temperature and stirred for overnight. Desired product could be detected by LCMS. The reaction mixture was used in the next step directly without further purification. LCMS: (ESI, m/z): [M+H]⁺=229

To a stirred solution of (2R)-2-amino-6-oxo-6-(piperidin-1-yl)hexanoic acid (3.63 g, 15.901 mmol, 1.00 equiv) in dioxane (150 mL) and NaHCO₃ (4.01 g, 47.703 mmol, 3 equiv) in H₂O (50 mL) was added 2,5-dioxopyrrolidin-1-yl 9H-fluoren-9-ylmethyl carbonate (6.44 g, 19.081 mmol, 1.2 equiv) at room temperature under nitrogen atmosphere. After be stirred overnight, the mixture was acidified to pH 1˜2 with concentrated hydrochloric acid. The resulting mixture was extracted with ethyl acetate (3×100 mL). The combined organic layers were dried over anhydrous Na₂SO₄ and concentrated under reduced pressure. The residue was purified by silica gel column chromatography, eluted with EA:PE (1:1) to afford (2R)-2-{[(9H-fluoren-9-ylmethoxy)carbonyl]amino}-6-oxo-6-(piperidin-1-yl)hexanoic acid (1.38 g, 19.01%) as a white solid. LCMS: (ESI, m/z): [M+H]⁺=451

### Building Block 67: Preparation of (S)-2-((((9H-fluoren-9-yl)methoxy)carbonyl)amino)-6-(4,4-difluoropiperidin-1-yl)hexanoic Acid

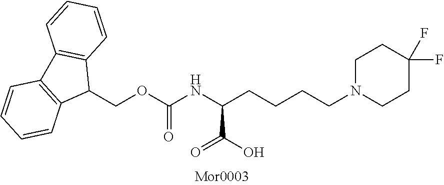 [SMILES: O=C(N[C@@H](CCCCN1CCC(F)(F)CC1)C(=O)O)OCC1c2ccccc2-c2ccccc21]

To a stirred mixture of methyl (2S)-2-[(tert-butoxycarbonyl) amino]-6-(methanesulfonyloxy)hexanoate (5 g, 14.732 mmol, 1.00 equiv) and 4,4-difluoropiperidine (1.96 g, 16.205 mmol, 1.1 equiv) in DMF (100 mL) was added KI (0.12 g, 0.737 mmol, 0.05 equiv) and DIPEA (7.62 g, 58.928 mmol, 4 equiv) dropwise at 15˜25° C. under nitrogen atmosphere. The resulting mixture was stirred for 24 h at 55˜60° C. under nitrogen atmosphere. After reaction completed, the mixture was concentrated under reduced pressure and filtered. The crude product was purified by Prep-HPLC to afford methyl (2S)-2-[(tert-butoxycarbonyl) amino]-6-(4,4-difluoropiperidin-1-yl)hexanoate (1.8 g, 33.53%) as a yellow oil. LCMS: (ESI, m/z): [M+H]⁺=365.22

Into a 250 mL round-bottom flask were added methyl (2S)-2-[(tert-butoxycarbonyl) amino]-6-(4,4-difluoropiperidin-1-yl) hexanoate (1.8 g, 4.939 mmol, 1.00 equiv) and conc. HCl (36 mL) at room temperature. The resulting mixture was stirred for 1 h at room temperature under nitrogen atmosphere. The resulting mixture was concentrated under reduced pressure. The crude product was used in the next step directly without further purification. LCMS: (ESI, m/z): [M+H]+=265.16

To a stirred solution of methyl (2S)-2-amino-6-(4,4-difluoropiperidin-1-yl) hexanoate (1.3 g, 4.918 mmol, 1.00 equiv) in THF (20 mL) and H₂O (20 mL) was added LiOH (0.35 g, 14.754 mmol, 3 equiv) in portions at room temperature under air atmosphere. The resulting mixture was stirred for 1 h at room temperature under nitrogen atmosphere. The resulting mixture was concentrated under reduced pressure to remove THF. The aqueous layer was acidified to pH 5˜6 with HCl (aq.) and then basified to pH 8 with NaHCO₃ solid. The final mixture was used in the next step directly without further purification. LCMS: (ESI, m/z): [M+H]⁺=251.15.

Into a dioxane (5.00 mL) were added 2,5-dioxopyrrolidin-1-yl 9H-fluoren-9-ylmethyl carbonate (2.72 g, 8.064 mmol, 1.1 equiv) at room temperature. The above solution was added into the mixture of the previous batch dropwise over 5 min at room temperature. The resulting mixture was stirred for additional 14 h at room temperature. The reaction mixture was acidified with dilute HCl and extracted with EtOAc. The organic layer was washed with brine, dried and concentrated in vacuo. The residue was purified by reverse flash chromatography with the following conditions: column, C18 silica gel; mobile phase, MeCN in water, 45% to 50% gradient in 10 min; detector, UV 220 nm. This resulted in (2S)-6-(4,4-difluoropiperidin-1-yl)-2-{[(9H-fluoren-9-ylmethoxy)carbonyl]amino}hexanoic acid (1.4938 g) as a white solid. LCMS: (ESI, m/z): [M+H]⁺=473.22.

### Building Block 68: Preparation of N2-(((9H-fluoren-9-yl)methoxy)carbonyl)-N6-(tert-butoxycarbonyl)-N2,N6-dimethyl-L-lysine

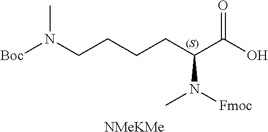

To a mixture of N6-(tert-butoxycarbonyl)-L-lysine, (1.50 kg, 6.09 mol, 1.00 eq) and benzaldehyde (646 g, 6.09 mol, 615 mL, 1 eq) in MeOH (15 L) was added TFA (34.7 g, 304 mmol, 22.5 mL, 0.05 eq) at 20-25° C. The mixture was stirred at 20-25° C. for 2 hours. MeOH (7.5 L) was added into the mixture. Then NaBH(OAc)₃ (2.84 kg, 13.4 mol, 2.20 eq) was added in ten portions at 25˜30° C. over 2 hrs. The mixture was stirred at 20˜25° C. for another 10 hrs. LCMS showed desired mass was detected. To the reaction mixture was added dropwise a solution of sat. aq. NH₄Cl (7.5 L) at 25˜30° C. for 75 mins. The residue was triturated with H₂O (15 L) and MTBE (30 L) at 20° C. for 30 min. The mixture was filtered and the filter cake was dried in the oven to give the product. N2-benzyl-N6-(tert-butoxycarbonyl)-L-lysine (1.75 kg, 5.16 mol, 84.7% yield, 99.0% purity) was obtained as a white solid, which confirmed by LCMS. LCMS: (ESI, m/z): [M+H]⁺=336.22.

To a mixture of N2-benzyl-N6-(tert-butoxycarbonyl)-L-lysine (1.70 kg, 5.00 mol, 99.0% purity, 1.00 eq) and formaldehyde (812 g, 10.0 mol, 745 mL, 37% purity, 2.00 eq) in MeOH (17 L) was added TFA (28.5 g, 250.13 mmol, 18.52 mL, 0.05 eq) at 25° C. The mixture was stirred at 25° C. for 0.5 hour. Then NaBH(OAc)₃ (2.33 kg, 11.01 mol, 2.2 eq) was added in ten portions at 25˜30° C. for 1 hrs. The mixture was stirred at 25° C. for 1 hrs. LCMS showed starting material was consumed completely and one main peak with desired mass was detected. The solution of sat. aq. NH₄Cl (3.4 L) was added drop-wise into the mixture at 25˜30° C. over 40 mins. Then the mixture was concentrated under reduced pressure to 7 L. The residue was extracted with EtOAc (4 L×3). The combined organic layers were washed with sat. aq. NaCl (3 L), dried over Na₂SO₄ (2.00 kg), filtered and concentrated under reduced pressure to give a residue. The residue was triturated with MTBE (11 L) at 25° C. for 30 mins, filtered and dried in oven to give N2-benzyl-N6-(tert-butoxycarbonyl)-N2-methyl-L-lysine (1.75 kg, crude) as a white solid, which was confirmed by LCMS (EC4247-24-P1A3). LCMS: (ESI, m/z): [M+H]⁺=351, RT=0.517 mins

To a solution of N2-benzyl-N6-(tert-butoxycarbonyl)-N2-methyl-L-lysine (600 g, 1.71 mol, 1.00 eq) in MeOH (5.00 L) was added Pd/C (30.0 g, 10% purity) and Pd(OH)₂ (30.0 g, 20% purity) under Ar atmosphere. The suspension was degassed and purged with Ar for 3 times. The mixture was stirred under H₂ (3 MPa) at 60° C. for 12 hrs. LCMS (EC4402-59-P1A2) indicated starting material was consumed completely. The reaction was filtered and concentrated in vacuum and combined with the cake. A suspension of the crude product N6-(tert-butoxycarbonyl)-N2-methyl-L-lysine (˜297 g) in H₂O (3.00 L) was used into next step.

To a solution of N6-(tert-butoxycarbonyl)-N2-methyl-L-lysine (297 g, 1.14 mol, 1.00 eq) in THF (1.50 L) and H2O (1.50 L) was added NaHCO₃ (287 g, 3.42 mol, 133 mL, 3.00 eq) and FMOC-OSU (462 g, 1.37 mol, 1.20 eq) at 0° C. and stirred at 15° C. for 16 hrs. TLC (PE:EA=1:1, R_{f}=0.23) indicated starting material was consumed completely. The reaction was acified with 1 M HCl to pH=5-6, extracted with EtOAc (2 L*2). The combined organic phase were dried over Na₂SO₄, filtered and concentrated in vacuum. The combined organic phase were washed with brine (1 L), dried over Na₂SO₄, filtered and concentrated in vacuum. The crude product was purified by column chromatography (SiO₂, PE:EA=10/1 to 0/1). N2-(((9H-fluoren-9-yl)methoxy)carbonyl)-N6-(tert-butoxycarbonyl)-N2-methyl-L-lysine (467 g, 0.93 mol, 81.37% yield, 96% purity) was obtained as a yellow gum. LCMS: RT=0.627 mins, MS+23=505

A solution of N2-(((9H-fluoren-9-yl)methoxy)carbonyl)-N6-(tert-butoxycarbonyl)-N2-methyl-L-lysine (350 g, 696 mmol, 96.0% purity, 1.00 eq) in dioxane (2 L) was added dropwise HCl/dioxane (4 M, 1.04 L, 6.00 eq) at 0° C. and stirred at 0° C. for 16 hrs. LCMS showed starting material was consumed completely and desired mass was detected. The reaction was filtered, the filtered cake was washed with MTBE (500 mL×2) and concentrated to give N2-(((9H-fluoren-9-yl)methoxy)carbonyl)-N2-methyl-L-lysine (227 g, 541 mmol, 88.3% yield) as a white solid. LCMS: RT=0.447 mins, MS+1=383

To a solution of N2-(((9H-fluoren-9-yl)methoxy)carbonyl)-N2-methyl-L-lysine (278 g, 663 mmol, 1.00 eq) in DCM (2250 mL) was added Me₃SiCl (216 g, 1.99 mol, 252 mL, 3 eq) and DIEA (343 g, 2.65 mol, 462 mL, 4.00 eq) at 25° C. and stirred at 50° C. for 2 hrs. Then the mixture was cold to 0-10° C. and DIEA (257 g, 1.99 mol, 346 mL, 3.00 eq) and TrtCl (222 g, 796 mmol, 1.20 eq) was added. The final reaction was stirred at 40° C. for 28 hrs. LCMS indicated starting material was consumed completely. The reaction mixture was concentrated in vacuum to remove DCM, diluted with 2.5 L of EtOAc, washed with sat. NaH₂PO₄ (1 L) and brine (1 L), dried over Na₂SO₄, filtered and concentrated in vacuum. N2-(((9H-fluoren-9-yl)methoxy)carbonyl)-N2-methyl-N6-trityl-L-lysine (423 g, crude) was obtained as a yellow gum and used into next step without purification. LCMS: RT=0.635 mins, MS+1=625

To a mixture of N2-(((9H-fluoren-9-yl)methoxy)carbonyl)-N2-methyl-N6-trityl-L-lysine (363 g, 581 mmol, 1.00 eq), NaH₂PO₄ (139 g, 1.16 mol, 2.00 eq) and HCHO (165 g, 2.03 mol, 151 mL, 37% purity, 3.50 eq) in DCM (3000 mL) was added NaBH(OAc)₃ (246.28 g, 1.16 mol, 2 eq) at 0° C. and stirred at 20° C. for 2 hrs. LCMS indicated starting material was consumed completely. The reaction mixture was washed with 3 L of water and 3 L of brine, dried over Na2SO4, filtered and concentrated in vacuum. N2-(((9H-fluoren-9-yl)methoxy)carbonyl)-N2,N6-dimethyl-N6-trityl-L-lysine (395 g, crude) was obtained as a yellow gum, used into next step without purification.

To a solution of N2-(((9H-fluoren-9-yl)methoxy)carbonyl)-N2,N6-dimethyl-N6-trityl-L-lysine (395 g, 618 mmol, 1.00 eq) in dioxane (2.50 L) was added HCl/dioxane (4 M, 618 mL, 4.00 eq) at 0° C. and stirred at 15° C. for 16 hrs. LCMS indicated starting material was consumed completely. The reaction mixture was concentrated in vacuum, poured into 3 L of MTBE and filtered. The cake was dried under reduced pressure to give the product. N2-(((9H-fluoren-9-yl)methoxy)carbonyl)-N2,N6-dimethyl-L-lysine hydrochloride (318 g, 691.18 mmol, 55.9% yield, 94.1% purity) was obtained as a yellow gum, which confirmed by LCMS. LCMS: RT=0.447 mins, MS+1=397

To a solution of N2-(((9H-fluoren-9-yl)methoxy)carbonyl)-N2,N6-dimethyl-L-lysine hydrochloride (297 g, 686 mmol, 1.00 eq) in THF (1000 mL) and H₂O (2000 mL) was added NaHCO₃ (172.89 g, 2.06 mol, 80.04 mL, 3 eq) and (Boc)₂O (179 g, 823 mmol, 189 mL, 1.20 eq) at 0° C. and the mixture was stirred at 15° C. for 12 hrs. LCMS indicated starting material was consumed completely. The reaction was acified by 1 M HCl to pH=5-6, extracted with EtOAc (1.5 L×2), washed with brine (2 L), dried over Na₂SO₄, filtered and concentrated in vacuum. The crude product was purified by column chromatography (SiO2, PE:EA=100/1 to 1/1, Plate 1, PE:EA=1:1, R_{f}=0.26). N2-(((9H-fluoren-9-yl)methoxy)carbonyl)-N6-(tert-butoxycarbonyl)-N2,N6-dimethyl-L-lysine (147 g, 582 mmol, 42.4% yield, 98.7% purity) was obtained as a light yellow solid, which confirmed by LCMS. LCMS: RT=0.661 mins, MS+23=519.

### Building Block 69: Preparation of (2S,4R)-1-(3,3-difluoro-1-(trifluoromethyl)cyclobutane-1-carbonyl)-4-fluoropyrrolidine-2-carboxylic Acid

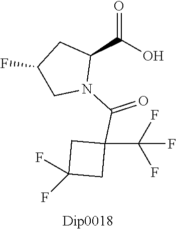 [SMILES: O=C(O)[C@@H]1C[C@@H](F)CN1C(=O)C1(C(F)(F)F)CC(F)(F)C1]

This compound was prepared following the general synthetic sequence described for the preparation of Building Block 6 using 3,3-difluoro-1-(trifluoromethyl)cyclobutane-1-carboxylic acid. ESI MS m/z 319.06.

### Building Block 70: Preparation of (2S,4R)-4-fluoro-1-(4-(trifluoromethyl)tetrahydro-2H-pyran-4-carbonyl)pyrrolidine-2-carboxylic Acid

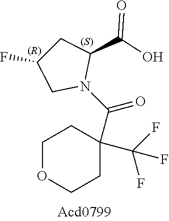

A mixture of methyl (2S,4R)-4-fluoropyrrolidine-2-carboxylate (1.6 g, 9.786 mmol, 1 equiv, 90%), 4-(trifluoromethyl)oxane-4-carboxylic acid (1.94 g, 9.786 mmol, 1.00 equiv), TCFH (4.12 g, 14.679 mmol, 1.50 equiv) and NMI (4.02 g, 48.930 mmol, 5 equiv) in ACN (30 mL) was stirred for 16 h at 25° C. under nitrogen atmosphere. The reaction mixture was directly purified by reverse flash chromatography with the following conditions: column, C18; mobile phase, Acetonitrile in water, 5% to 60% gradient in 25 min; detector, UV 220 nm. This resulted in methyl (2S,4R)-4-fluoro-1-(4-(trifluoromethyl)tetrahydro-2H-pyran-4-carbonyl)pyrrolidine-2-carboxylate (2.2 g, 68.69%) as a white solid. LCMS: (ESI, m/z): [M+H]⁺=328.

A mixture of methyl (2S,4R)-4-fluoro-1-(4-(trifluoromethyl)tetrahydro-2H-pyran-4-carbonyl)pyrrolidine-2-carboxylate (3.4 g, 10.389 mmol, 1 equiv) and NaOH (2.08 g, 51.945 mmol, 5.0 equiv) in MeOH (80 mL)/H₂O (30 mL) was stirred for 16 h at 20° C. The organic solvents were evaporated in vacuo and the water was acidified by 1N HCl. The resulting precipitation was collected by filtration and dried in air. This resulted in (2S,4R)-4-fluoro-1-(4-(trifluoromethyl)tetrahydro-2H-pyran-4-carbonyl)pyrrolidine-2-carboxylic acid (3.2509 g, 97.52%) as a white solid. LCMS: (ESI, m/z): [M+H]⁺=314.0.

### B. Solid Phase Synthesis, Cleavage, and Cyclization to Prepare Compounds of Formula I

The compounds of Formula I described herein can be prepared as described herein. Generally, monomeric Building Blocks, described above, are covalently linked via solid phase synthesis to form an on resin linear peptide, followed by cleavage and in solution cyclization. Additional transformations to prepare compounds of Formula I often include, but are not limited to alkylation, deprotection, cleavage from solid phase resin, and cyclization.

The following paragraphs and subheadings provide general comments and procedures on how the compounds of Formula I were prepared.

Table 2A and B, provided below, list the Building Blocks and procedures used to prepare the listed exemplified compounds of Formula I. The Building Blocks in Table 2A and B are listed using a Short Hand Name that is identified in Table 1. The procedures in Table 2A and B are listed using the abbreviations identified in the subheadings below.

The solid phase linear synthesis of peptides containing N-alkylated amino acid monomers was successfully completed either by using pre-N-alkylated amino acid building blocks or by a method of sequential on-resin Mitsunobu alkylation (Chatterjee et al., Synthesis of N-methylated cyclic peptides. Nature Protocols, Vol 7, 432-444, 2012).

Certain compounds of Formula I described herein contain building blocks with sidechains that were altered on resin after incorporation into the linear peptide. Exemplary methods are described in the following paragraphs. See, for example, Example 3 wherein the sidechain of Res5 (KDde) is deprotected and functionalized with a morpholine moiety (Building Block: B2BE); Example 10 wherein the sidechain Res4 (KDde) is deprotected and functionalized with a morpholine moiety (Building Block: B2BE); Example 216 wherein Res6 (KDde) is deprotected and functionalized with deuterated methyl group (MeOD); and Example 308 wherein Res5 (ODde) is deprotected and functionalized with an acyl moiety (RA245).

The proper choice of functionalized solid support allows for sufficient resin loading and a C-terminal carboxylic acid functionality. Generally, the solid support used herein is derived from polystyrene crosslinked with divinylbenzene and functionalized by means of the 2-chlorotrityl linker.

The solid phase peptide synthesis methods described in this document can be carried out manually or automated using specialized liquid handlers.

When carried out as a parallel array synthesis on a Biotage Syro II automated peptide synthesizer or manually, the processes of the disclosure can be advantageously carried out as described herein, but it will be immediately apparent to those skilled in the art how these procedures can be modified to synthesize a single compound of the disclosure on multi-gram scale.

A number of reaction vessels equal to the total number of compounds to be synthesized by the parallel method are loaded with 50-150 mg of the appropriate functionalized solid support, preferably polystyrene 2-chlorotrityl chloride resin.

The solvent to be used must be capable of swelling the resin and includes, but is not limited to, dichloromethane (DCM), dimethylformamide (DMF), N-methylpyrrolidone (NMP), dioxane, toluene, tetrahydrofuran (THF), ethanol (EtOH).

Linear peptides can be cleaved from the 2-chlorotrityl chloride resin under mild acidic conditions (24% HFIP in DCM) without removing acid-labile sidechain protecting groups (Pbf, Boc). Alternatively, more harsh cleavage conditions can be applied (20% TFA/DCM, or 95% TFA/2.5% H20/2.5% TIS) to remove Boc, Mtt, and Trt, or Pbf and tBu respectively, during resin cleavage.

The 9-fluorenylmethoxycarbonyl (Fmoc)-protected amino acid derivatives are preferably used as the building blocks for the construction of the compounds of Formula I in this disclosure. For the deprotection, i.e. Fmoc removal, 20% piperidine in DMF or 2% DBU/2% piperidine in DMF can be used. It is understood that alternative protecting groups may be used.

The quantity of the reactant, i.e. of the amino acid derivative, is usually 1 to 20 equivalents based on the milliequivalents per gram (meq/g) loading of the functionalized solid support (typically 0.3 to 1.4 m eqv/g for 2-chlorotrityl chloride polystyrene resin). Originally weighed into the reaction vessel. Additional equivalents of reactants can be used, if required, to drive the reaction to completion in a reasonable time. The preferred workstation (without, however, being limited thereto) is Biotage's Syro II synthesizer equipped with a transfer unit and a reservoir box used during the resin cleavage step. The synthesizer is able to provide a controlled environment, for example, reactions can be accomplished at elevated temperatures and under inert gas if desired.

Amide bond formation is facilitated by the activation of the alpha-carboxyl group for the acylation step. Excess coupling reagent and base, on the order of 2 to 24 molar equivalents may be used to push the coupling reaction to completion. Amino acid couplings onto non-alkylated or N-Methylated amino termini are most commonly achieved via HATU coupling. Amino acid couplings onto highly sterically-hindered N-alkylated amino termini are achieved via DIC-mediated coupling. Since near-quantitative coupling reactions are highly preferred, it is desirable to have experimental evidence for completion of the reactions. The ninhydrin test or regular reaction checking by LCMS are critical to confirm the absence of uncoupled starting material on resin. In order to couple highly acidic or difficult to activate carboxylic acids onto the N-terminus of a growing peptide chain, alternative methods have been developed, which utilize K-Oxyma (CAS #158014-03-0) as an activating agent/and or the maintenance of a narrow pH during the reaction.

The on-resin alkylation of alpha amino groups on the solid phase is known in the art. The procedure for introducing a methyl group (described in Chatterjee et al., Synthesis of N-methylated cyclic peptides. Nature Protocols, 2012, Vol 7, 432-444) can be accomplished, for example, by 1) protecting the N-terminal amine with a 2-nosyl group, 2) Mistunobu alkylation with Methanol, Triphenylphosphine, and DIAD or related reagent, and 3) deprotection of the 2-nosyl group with DBU and a thiol such as mercaptoethanol. Some cyclic peptides in this disclosure were accessed using a variation of the published on-resin Mitsunobu method to append larger primary alcohols to activated amino groups (on the backbone or sidechain) on the solid phase as an alternative to the more widely used reductive amination approach (Pels et al., Solid-Phase Synthesis of Diverse Peptide Tertiary Amides by Reductive Amination. ACS Combinatorial Science, 2015, 17, 3, 152-155).

Following each reaction, the resin-bound intermediate within each reaction vessel is washed free of excess or retained reagents, of solvents, and of by-products by repetitive exposure to pure solvents (DCM, DMF, or MeOH depending on the reaction). The reaction vessels are filled with solvent (preferably 5 mL), agitated for 1 minute, and drained to expel the solvent, and the process is repeated twice more.

The above described procedure of reacting the resin bound compound with reagents within the reaction tubes followed by removal of excess reagents, by-products, and solvents is repeated with each successive transformation until the desired resin-bound fully protected linear peptide has been obtained.

For the modification of sidechains along the linear peptide, including but not limited to sidechain acylation and alkylation, residues with sidechains decorated with base-stable protecting groups such as Dde or 2-Nosyl, are used. Upon the completion of the linear synthesis the orthogonally protected sidechains are deprotected and modified with subsequent chemistries. Dde-protected sidechains can be removed on-resin with the use of 10% hydrazine in DMF. The resulting primary amine at the branch point serves as a substrate in subsequent on-resin acylation, reductive amination, or alkylation reactions. 2-Nosyl-protected sidechains can be N-alkylated via the Mitsunobu conditions described above, followed by removal of the 2-Nosyl group, to yield a secondary amine.

Detachment of the fully protected linear peptide from the solid support is achieved by exposing the loaded resin with a solution of the reagent used for cleavage (preferably 3 to 5 mL). Temperature control, agitation, and reaction monitoring are implemented as described above. Via a transfer unit, the reaction vessels are connected with a reservoir box containing reservoir tubes to efficiently collect the cleaved product solutions. The resins remaining in the reaction vessels are then washed 2 to 5 times as above with 3 to 5 mL of an appropriate solvent to extract as much of the detached products as possible. The product solutions thus obtained are combined, taking care to avoid cross-mixing. The individual solutions/extracts are then manipulated as needed to isolate the final compounds. Typical manipulations include, but are not limited to, evaporation, concentration, liquid/liquid extraction, acidification, basification, neutralization, or additional reactions in solution.

The solutions containing fully deprotected linear peptides are then evaporated, resuspended in DMSO, purified via RP-HPLC, and lyophilized.

Cyclization is conducted on the lyophilized linear peptide. Cyclization can be achieved using a variety of cyclization reagents (e.g., PyBop, PyAop, HATU, HBTU, T3P) in a variety of pure or mixed solvents (e.g., ACN/THF, NMP DCM, DMF, EtOAc, etc) at a variety of concentrations. To facilitate rapid cyclization, low dimer formation, and facile purification of the macrocycles described herein, 3 eq T3P, 8 eqv DIEA, in 1.5 mL DCM:NMP is preferred. At small scale (50 umol), the reaction is typically complete within 10 minutes. Larger scale reactions are diluted in volumes up to 250 mL and are allowed to react for up to 12 hours. The progress of the reaction is followed using LCMS to monitor disappearance of starting materials. Upon completion of the reaction, excess solvent is removed by evaporation and the compounds are purified by RP-HPLC and lyophilized.

### 1. Solid Phase Synthesis—General Methods

The general methods i-xiv were generally performed on a 50 μmol scale reactions on 50-100 mg of 2-chlorotritylchloride polystyrene resin.

i. CTC—Resin Loading

Fmoc-AA-OH (4 equiv.) was dissolved in 1.0 mL of anhydrous NMP. Neat DIEA (8 equiv.) was added to the Fmoc-AA-OH solution. The solution was dispensed in a peptide reactor vessel containing 100 mg of 2-chlorotrityl chloride (CTC) resin and was agitated for 2 hours at room temperature. The Fmoc-AA-OH solution was drained then the resin was washed with 1.0 mL DMF three times. Unreacted CTC resin was capped with 1.0 mL solution of methanol:DMF (50:50), and DIEA (8 equiv.) for 10 minutes at room temperature. The methanol solution was drained then the resin was washed with 1.0 mL DMF three times.

Following complete coupling, the Fmoc protecting group was displaced using method ii.

ii. Fmoc Deprotection

A mixture of piperidine:DMF (20:80, 1 mL) was added to the resin and agitated for 10 to 15 minutes at room temperature. The piperidine solution was drained then the resin was washed with 1.0 mL DMF three times.

iii. HATU—Peptide Coupling, Followed by Fmoc Deprotection.

A solution of Fmoc-AA-OH (4 equiv.), HATU (4 equiv.), and DIEA (8 equiv) in 1.0 mL of anhydrous NMP was prepared. The mixture was allowed to react at room temperature for 5 minutes then was added to the resin and was agitated at 35 to 45° C. for 10 to 90 minutes. The mixture was drained then the resin was washed with 1.0 mL of DMF three times.

If the reaction was incomplete (less than 95% coupled, as determined by LCMS), or if the coupling was performed on an N-methylated amine substrate, the coupling was repeated a second time.

Following complete coupling (as determined by LCMS), the Fmoc protecting group was displaced using method ii.

iv. HATUnf—Peptide Coupling, No Fmoc Deprotection

A solution of Carboxylic acid or Fmoc-AA-OH (4 equiv.), HATU (4 equiv.), and DIEA (8 equiv) in 1.0 mL of anhydrous NMP was prepared. The mixture was allowed to react at room temperature for 5 minutes then was added to the resin and was agitated at 35 to 45° C. for 10 to 90 minutes. The mixture was drained then the resin was washed with 1.0 mL of DMF three times.

If the reaction was incomplete (as determined by LCMS), or if the coupling was performed on an N-methylated amine substrate the coupling was repeated a second time.

v. KO—Sterically-Hindered Peptide Coupling, Followed by Fmoc Deprotection

Fmoc-AA-OH or Carboxylic acid (4 equiv.), K-Oxyma (3.8 equiv.), and DIC (3.8 equiv.) was dissolved in 1.0 mL anhydrous NMP. The mixture was allowed to react at room temperature for 5 minutes then was added to the resin and was agitated at 35 to 45° C. for 10 to 90 minutes. The mixture was drained then the resin was washed with 1.0 mL of DMF three times. The method was repeated twice.

Following complete coupling (as determined by LCMS), the Fmoc protecting group was displaced using method ii.

vi. EEDQ—Sterically-Hindered Peptide Coupling, Followed by Fmoc Deprotection

Coupling on N-alkylated amines when N-alkyl group is larger than N-methyl. Fmoc-AA-OH (6 equiv) and EEDQ (5 equiv.) were dissolved in 1.0 mL of anhydrous NMP. The mixture was reacted for 15 minutes. Then, the mixture was added to the resin and was agitated for 3 hours at 45° C. The mixture was drained then the resin was washed with 1.0 mL of DMF three times. The method was repeated twice.

Fmoc protecting group was displaced using method ii.

vii. DIC—Sterically-Hindered Peptide Coupling, Followed by Fmoc Deprotection

Coupling on N-alkylated amines when N-alkyl group is larger than N-methyl. Fmoc-AA-OH (24 equiv.) was dissolved in 1.5 mL of anhydrous NMP:DCE (50:50). NMP may be added dropwise to dissociate Fmoc-AA-OH completely. DIC (23 equiv.) was added to the Fmoc-AA-OH solution. The mixture was added to the resin and was agitated for 12 to 24 hours at room temperature. The slurry was drained then the resin wash washed with 1.0 mL of methanol four times and 1.0 mL of DMF three times.

If the reaction was incomplete (less than 95% coupling as determined by LCMS), the coupling was performed a second time.

Following complete coupling, the Fmoc protecting group was displaced using method ii.

viii. DIC_KMe2—Neutral Peptide Coupling Used for KMe2 Incorporation.

Fmoc-KMe2—OH (4 equiv.) was dissolved in 1 mL of anhydrous NMP. DIC (4 equiv.) was added to the Fmoc-KMe2-OH solution. The mixture was added to the resin and was agitated for 2 hours at room temperature. The slurry was drained then the resin was washed with 1.0 mL of methanol three times and 1.0 mL of DMF three times.

Fmoc protecting group was displaced using method ii.

ix. Onto_KMe2—Peptide Coupling Used to Couple Amino Acid onto KMe2 Residue.

A solution of Fmoc-AA-OH (4 equiv.), HATU (4 equiv.), and DIEA (8 equiv) in 1.0 mL of anhydrous NMP was prepared. The mixture was allowed to react at room temperature for 5 minutes then was added to the resin and was agitated at 25° C. for 10 to 90 minutes. The mixture was drained then the resin was washed with 1.0 mL of DMF three times.

Following complete coupling, the Fmoc protecting group was displaced using method ii.

x. DdeR—Dde Removal Via Hydrazine

10% hydrazine monohydrate in in 1.0 mL NMP was added to the resin and was agitated for 20 minutes at room temperature. The mixture was drained then the resin was washed with 1.0 mL DMF three times.

xi. RA—Reductive Amination.

Aldehyde (20 equiv.) was dissolved in 1.0 mL of anhydrous NMP. The mixture was added to the resin and was agitated for 3o minutes at room temperature. Then, the mixture was drained and the resin was washed with 1.0 mL of DMF three times.

1.0 mL of DCM:MeOH (3:1) was added to the resin. Then, sodium borohydride (NaBH₄, 20 equiv.) was added to the resin. The slurry was agitated for 1 hour at room temperature. The slurry was drained and the resin was washed with 1.0 mL of methanol six times then 1.0 mL of DMF three times.

xii. MITS—Nosylation, Mitsunobu, Nosyl Deprotection

Nosyl protection. 2,6-lutidine (6 equiv.) dissolved in 0.5 mL of anhydrous DCE was added to the resin. 2-nitrobenzenesulfonyl chloride (5 equiv.) dissolved in 0.5 mL anhydrous toluene was added to the resin then was agitated at 40 to 45° C. for 10 to 15 minutes. The mixture was drained then the resin was washed with 1.0 mL of anhydrous toluene three times. The method was repeated twice.

Alkylation via mitsunobu conditions. Triphenylphosphine (10 equiv.) dissolved in 0.7 mL anhydrous toluene was added to the resin. The appropriate primary alcohol (20 equiv. Of methanol, ethanol, propanol, butanol, or other) was added to the resin suspension. Azodicarboxylate (10 equiv) was added to the resin and the suspension was agitated at 35 to 45° C. for 15 to 30 minutes. The mixture was drained then the resin was washed with 1.0 mL of anhydrous DMF three times. The method was repeated twice.

Nosyl deprotection. 2-mercaptoethanol (5 equiv.) and 1,8-Diazabicyclo[5.4.0]undec-7-ene (5 equiv.) in 1.0 mL NMP was added to the resin and was agitated at 35 to 45° C. for 15 to 30 minutes. The mixture was drained then the resin was washed with 1.0 mL of anhydrous DMF three times. The method was repeated twice.

xiii. Morph—Conversion of a Primary Amine to a Morpholine Moiety

Bis(2-bromoethyl) ether (10 equiv.) dissolved in 1 mL of anhydrous NMP was added to the resin and was agitated at room temperature for 12 to 24 hours. The mixture was drained then the resin was washed with 1.0 mL of anhydrous DMF three times.

xiv. Ac—Acetylation of Amines

A solution of Acetic anhydride:DIEA:DMF (10:20:70, 1 mL) was added to the resin and was allowed to react at room temperature for 1 hour. The mixture was drained then the resin was washed with 1.0 mL of DMF three times.

### 2. Resin Cleavage, QC, and Linear Peptide Purification—General Methods

### xv. 20% TFA—Resin Cleavage

A solution of 20% TFA and 5% TIPS in DCM (2 mL) was added to the 50-100 mg of polystyrene resin in a solid phase reaction vessel. The contents of the vessel were shaken for one hour. The liquid phase of the reaction was filtered into a 50 mL conical vial. The cleaved resin was washed with an additional DCM (2 mL) and the wash was collected in the conical vial. Toluene (2 mL) was added to the cleaved peptide solution and the solution was either neutralized with triethylamine and concentrated under reduced atmosphere in a Genevac, or it was concentrated without neutralization on a rotary evaporator.

### xvi. 30% TFA—Resin Cleavage

A solution of 30% TFA and 5% TIPS in DCM (2 mL) was added to the 50-100 mg of polystyrene resin in a solid phase reaction vessel. The contents of the vessel were shaken for one hour. The liquid phase of the reaction was filtered into a 50 mL conical vial. The cleaved resin was washed with an additional DCM (2 mL) and the wash was collected in the conical vial. Toluene (2 mL) was added to the cleaved peptide solution and the solution was either neutralized with triethylamine and concentrated under reduced atmosphere in a Genevac, or it was concentrated without neutralization on a rotary evaporator.

xvii. 90% TFA—Resin Cleavage

A solution of 90% TFA and 5% TIPS in DCM (2 mL) was added to the 50-100 mg of polystyrene resin in a solid phase reaction vessel. The contents of the vessel were shaken for one hour. The liquid phase of the reaction was filtered into a 50 mL conical vial. The cleaved resin was washed with an additional DCM (2 mL) and the wash was collected in the conical vial. Toluene (2 mL) was added to the cleaved peptide solution and the solution was either neutralized with triethylamine and concentrated under reduced atmosphere in a Genevac, or it was concentrated without neutralization on a rotary evaporator.

xviii. 24% HFIP—Resin Cleavage

A solution of 24% HFIP and 2% TIPS in DCM (2 mL) was added to the 50-100 mg of polystyrene resin in a solid phase reaction vessel. The contents of the vessel were shaken for one hour. The liquid phase of the reaction was filtered into a 50 mL conical vial. The cleaved resin was washed with an additional DCM (2 mL) and the wash was collected in the conical vial and concentrated.

xix. Linear Peptide Mass Spec QC Method:

The quality control of linear peptides is performed on an Acquity UPLC with a single quad QDa mass detector system The method used is a 10-100 gradient with a flow rate of 0.8 milliliters per minute with a run time of 1.5 minutes. The solvents used are 0.1% formic acid in acetonitrile and 0.1% formic acid in water. The method starts at 10% of the acetonitrile solution until 0.2 minutes then the run ramps to 100% of the acetonitrile solution over the course of 0.5 minutes. The run then holds the 100% acetonitrile solution for 0.6 minutes then ramps down to 10% of the acetonitrile solution in 0.1 minutes. The 10% solution is held for an additional 0.1 minutes then the method is complete. The data for the vials are spot checked for the desired product and moved forward with purification.

xx. Linear Peptide Purification

The linear compounds are purified on a Xbridge C18 column with 10 mm by 150 mm dimensions using a prep Waters HPLC system in a dual column set up. Components of the Waters HPLC system include Waters 2767 Sample Manager, Waters 1525 Binary HPLC Pump, Waters 2545 Binary Gradient Module, Waters SFO System Fluidics Organizer, 515 HPLC Pump, Waters QDA and Waters 2998 Photodiode Array Detector. The wash solvent used to draw and rinse the syringe and needle is 30:70 acetonitrile:water. The 515 HPLC Pump uses optima fine methanol with 0.1% TFA. The solvent systems used for the gradient are solvent A: water with 0.1% TFA and solvent B: acetonitrile with 0.1% TFA. The method is ran based off a 30-95% gradient of solvent B for a 10-minute run at 7 milliliters per minute. The loading of the compound begins at 10% of solvent B for 2 minutes then ramps to 30% solvent B to commence the run and the method progressively ramps to 95% solvent B over the course of 8 minutes. The linear compounds are monitored using the Waters QDA and Waters 2998 Photodiode Array Detector. During the run a second column is washed using a regen pump on a 10-minute run at 4 milliliters per minute. The wash method is 6 minutes solvent B at 100% then ramped to 5% solvent B for 1 minute then for 3 minutes solvent B is held at 5%. Fractions containing the desired product are combined and frozen then placed onto lyophilizer until dry. Once linear purified compounds have dried, they can progress forward in the process to cyclization.

### 3. Cyclization & Post Cyclization Modifications—General Methods

xxi. T3P—Cyclization in the Absence of Hydroxyl Groups

T3P Method A, Small volume cyclization—the deprotected and purified linear product from a ˜50 umol reaction was dissolved in NMP (500 uL), DIEA (250 uL), and DCM (0.75 mL). T3P (31 uL, 3 eqv) is added, the solution is shaken and allowed to react for 1-10 minutes at room temperature. Reaction completion is confirmed via m/z on the Acquity UPLC instrument.

T3P Method B, Medium volume cyclization—the deprotected and purified linear product from a ˜50-200 umol synthesis is transferred to a 50 mL conical vial and dissolved in 1 mL NMP followed by the addition of DIEA (0.5 mL) and DCM (35 mL). T3P (3 eqv) is added to the solution and the reaction pH is adjusted to pH 9 or greater via dropwise addition of DIEA. The closed conical vial is then shaken at room temperature for 2 hours at 150 rotations per minute. The conical vials are then uncapped and the solutions are concentrated at 45 degrees Celsius under reduced pressure in a Genevac system. The evaporated crude material is then redissolved in acetonitrile for purification.

Optional T3P method for ˜200 μmol+scale synthesis, Large volume cyclization—the deprotected and purified linear product from a ˜200-400 umol synthesis is transferred to a 500 mL round bottom flask with a stir bar, and dissolved in DCM (250 mL). DIEA (3 eqv) is added to the flask, followed by T3P (3 eqv). The pH is adjusted to 9 with DIEA. The reaction is stirred at room temperature for 2-12 hours and monitored for reaction completion.

xxii. PyBop—Cyclization in the Presence of Hydroxyl Groups

PyBop Method A, Medium volume cyclization—the deprotected and purified linear product from a ˜50 umol synthesis is transferred to a 50 mL conical vial and dissolved in 1 mL NMP followed by the addition of DIEA (0.5 mL) and DCM (35 mL). PyBop (3 eqv) is added to the solution and the reaction pH is adjusted to pH 9 or greater via dropwise addition of DIEA. The closed conical vial is then shaken at room temperature for 2 hours at 150 rotations per minute. The conical vials are then uncapped and the solutions are concentrated at 45 degrees Celsius under reduced pressure in a Genevac system. The evaporated crude material is then redissolved in acetonitrile for purification.

PyBop Method B, Large volume cyclization—the deprotected and purified linear product from a ˜100-400 umol synthesis is transferred to a 500 mL round bottom flask with a stir bar, and dissolved in DCM (250 mL). DIEA (3 eqv) is added to the flask, followed by PyBop (3 eqv). The pH is adjusted to 9 with DIEA. The reaction is stirred at room temperature for 2-12 hours and monitored for reaction completion.

xxiii. Solution Deprotection

Boc—Boc-protected macrocycle (usually ˜5-50 mg) was dissolved in 25% TFA in DCM (5 mL). The reaction was monitored by LCMS for the disappearance of the starting material (usually ˜30 min). Upon completion, the reaction was concentrated. The crude oil was co-evaporated with DCE (5 mL×2). Crude product was then purified via RP-HPLC to yield the pure material for assay.

tBu—Tert-butyl-protected macrocycle (usually ˜5-50 mg) was dissolved in 60% TFA. 5% TIPS, in DCM (5 mL). The reaction was monitored by LCMS for the disappearance of the starting material (usually 30 min). Upon completion, the reaction was concentrated. The crude oil was co-evaporated with DCE (5 mL×2). Crude product was then purified via RP-HPLC to yield the pure material for assay.

### 4. Purification—General Methods

Cyclic compounds are purified using the mass-triggered Waters HPLC system described in linear purification section running a 10 minute reverse-phase gradient, Mobile phase A: Water, Mobile phase B: Acetonitrile, with 0.05% formic acid. An exemplary purification gradient is shown below:

| Time | Flow | | |
|---|---|---|---|
| (min) | (mL/min) | % A | % B |
| Initial | 15.00 | 80.0 | 20.0 |
| 1.60 | 15.00 | 80.0 | 20.0 |
| 2.00 | 20.00 | 80.0 | 20.0 |
| 7.50 | 20.00 | 45.0 | 55.0 |
| 8.00 | 20.00 | 0.0 | 100.0 |
| 9.50 | 20.00 | 0.0 | 100.0 |
| 9.75 | 20.00 | 70.0 | 30.0 |
| 10.00 | 20.00 | 70.0 | 30.0 |

### 5. High-Level Overview of Compound Synthesis

The scheme below provides a high-level summary of the methods used to prepare the compounds of Formula I described herein. Transformations 1-15 prepare linear intermediate compounds bound to a solid phase resin. Transformations 16-18 cleave the linear intermediate compound from the solid phase resin, cyclize the intermediate compound, and deprotect certain functional groups, if needed. Further details regarding transformations 1-18 are described in the following sections.

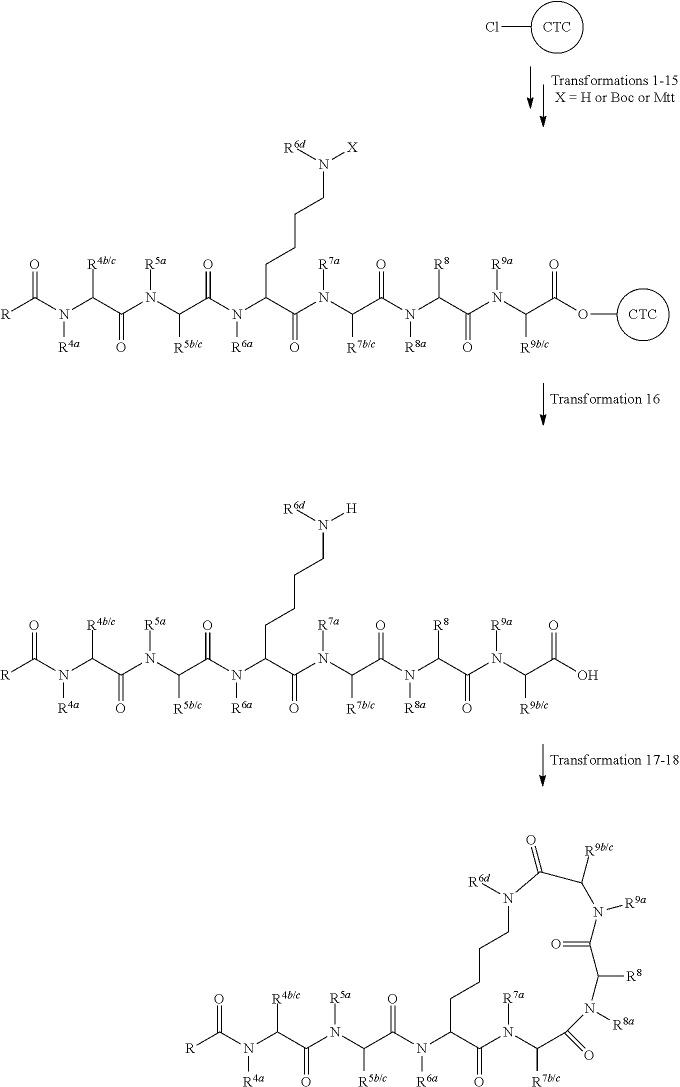

### 6. Synthesis of Example 456

To further illustrate the above sections and the synthesis of the compounds of Formula I described herein, the scheme and paragraphs below provide a start to finish synthetic route for an exemplary compound in this disclosure. Reference is made to “Transformation 1,” “Transformation 3,” etc. These are further detailed in the section below.

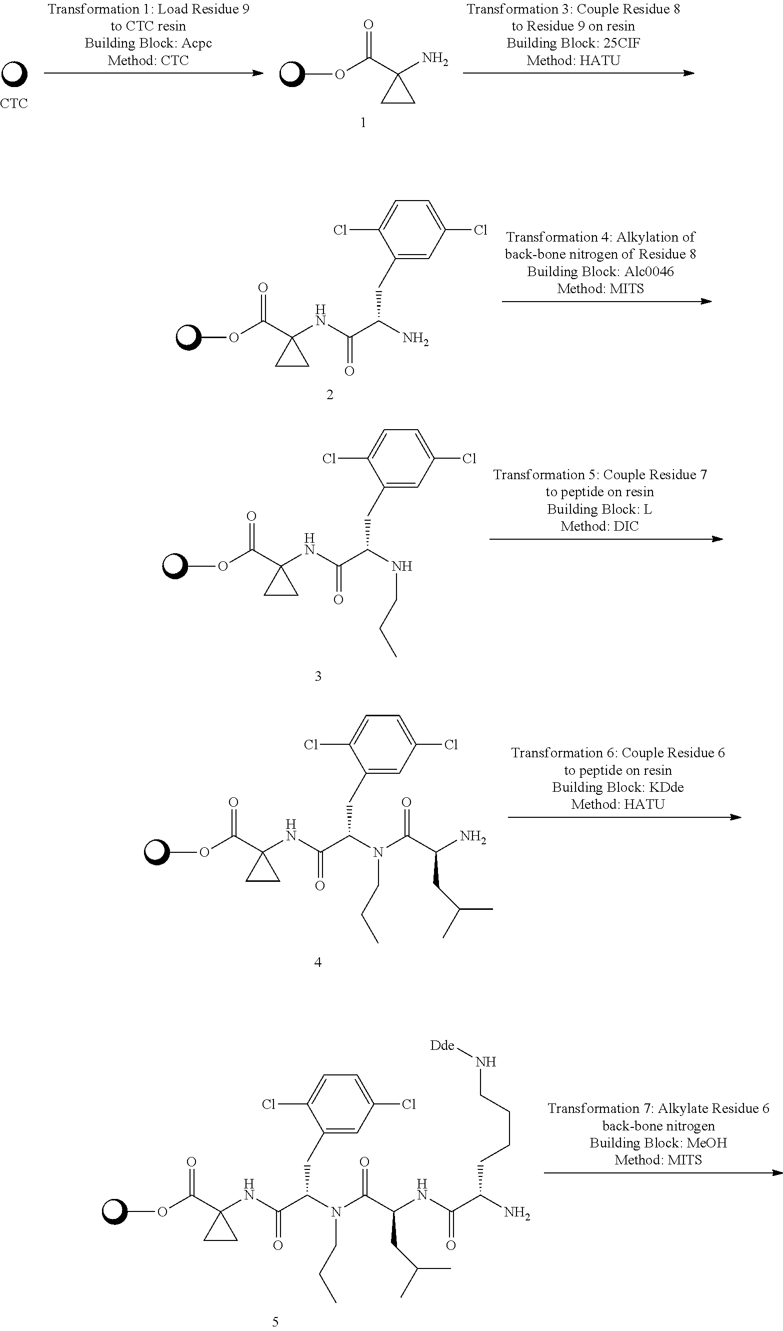

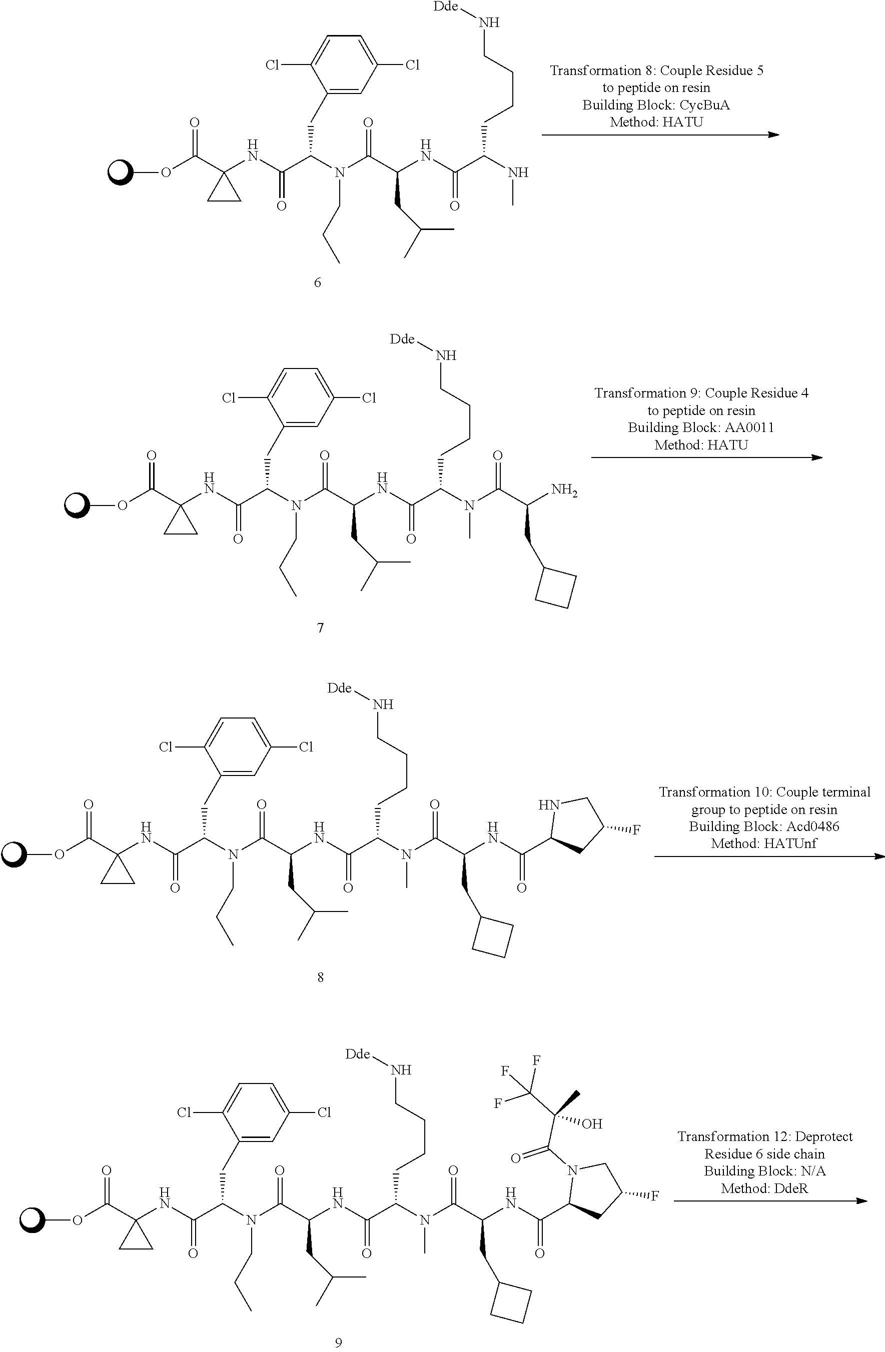

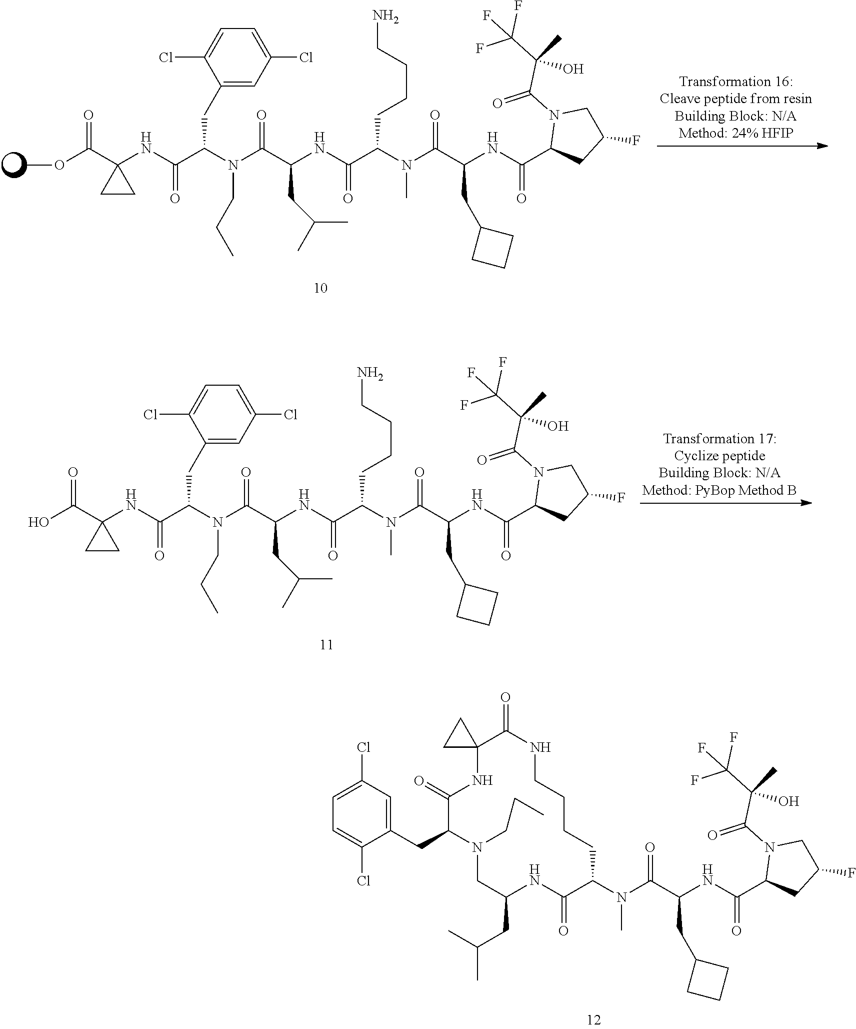 [SMILES: CCCN(C(=O)[C@H](CC(C)C)NC(=O)[C@H](CCCCN)N(C)C(=O)[C@H](CC1CCC1)NC(=O)[C@@H]1C[C@@H](F)CN1C(=O)[C@@](C)(O)C(F)(F)F)[C@@H](Cc1cc(Cl)ccc1Cl)C(=O)NC1(C(=O)OC)CC1 ; CCCN(C(=O)[C@H](CC(C)C)NC(=O)[C@H](CCCCN)N(C)C(=O)[C@H](CC1CCC1)NC(=O)[C@@H]1C[C@@H](F)CN1C(=O)[C@@](C)(O)C(F)(F)F)[C@@H](Cc1cc(Cl)ccc1Cl)C(=O)NC1(C(=O)O)CC1 ; CCCN1C[C@H](CC(C)C)NC(=O)[C@@H](N(C)C(=O)[C@H](CC2CCC2)NC(=O)[C@@H]2C[C@@H](F)CN2C(=O)[C@@](C)(O)C(F)(F)F)CCCCNC(=O)C2(CC2)NC(=O)[C@@H]1Cc1cc(Cl)ccc1Cl]

Synthesis of 1. (Method: CTC) Fmoc-1-aminocyclopropane-1-carboxylic acid (Acpc), CAS #126705-22-4, (4 equiv.) was dissolved in 1.0 mL of anhydrous NMP. Neat DIEA (8 equiv.) was added to the Fmoc-amino acid solution. The solution was dispensed in a peptide reactor vessel containing 100 mg of 2-chlorotrityl chloride (CTC) resin and was agitated for 2 hours at room temperature. The amino acid solution was drained then the resin was washed with 1.0 mL DMF three times. Unreacted CTC resin was capped with 1.0 mL solution of methanol:DMF (50:50), and DIEA (8 equiv.) for 10 minutes at room temperature. The methanol solution was drained then the resin was washed with 1.0 mL DMF three times. To remove Fmoc, A mixture of piperidine:DMF (20:80, 1 mL) was added to the resin and agitated for 10 to 15 minutes at room temperature. The piperidine solution was drained and then the resin was washed with 1.0 mL DMF three times.

Synthesis of 2. (Method: HATU) A solution of Fmoc-L-2,5-dichlorophenylalanine-OH (25ClF), CAS #1260614-80-9, (4 equiv.), HATU (4 equiv.), and DIEA (8 equiv) in 1.0 mL of anhydrous NMP was prepared. The mixture was allowed to pre-activate at room temperature for 5 minutes, and then was added to the resin and agitated at 35° C. for 30 minutes. The mixture was drained then the resin was washed with 1.0 mL of DMF three times. To remove Fmoc, A mixture of piperidine:DMF (20:80, 1 mL) was added to the resin and agitated for 10 to 15 minutes at room temperature. The piperidine solution was drained and then the resin was washed with 1.0 mL DMF three times.

Synthesis of 3. (Method: MITS) Three steps are required to mono-ethylate the terminal amine. 1) Nosyl protection. A solution of 2,6-lutidine (6 equiv.) dissolved in 0.5 mL of anhydrous DCE was added to the resin. 2-nitrobenzenesulfonyl chloride (5 equiv.) dissolved in 0.5 mL anhydrous toluene was added to the resin then was agitated at 40 to 45° C. for 10 to 15 minutes. The mixture was drained and then the resin was washed with 1.0 mL of anhydrous toluene three times. The method was repeated twice. 2) Alkylation via mitsunobu conditions. Triphenylphosphine (10 equiv.) dissolved in 0.7 mL anhydrous toluene was added to the resin. Dry propanol (Alc0046), (20 equiv.) was added to the resin. Azodicarboxylate (10 equiv) was added to the resin and was agitated at 45° C. for 30 minutes. The mixture was drained and then the resin was washed with 1.0 mL of anhydrous DMF three times. Alkylation was repeated twice. 3) Nosyl deprotection. 2-mercaptoethanol (5 equiv.) and 1,8-Diazabicyclo[5.4.0]undec-7-ene (5 equiv.) in 1.0 mL NMP was added to the resin and was agitated at 45° C. for 10 minutes. The mixture was drained and then the resin was washed with 1.0 mL of anhydrous DMF three times. Deprotection of the nosyl group was repeated twice.

Synthesis of 4. (Method: DIC), Fmoc-L-Leucine-OH (L), CAS #35661-60-0 (12 equiv.) was dissolved in 1.5 mL of anhydrous NMP:DCE (50:50). NMP may be added dropwise to dissociate Fmoc-AA-OH completely. DIC (12 equiv.) was added to the Fmoc-Leucine-OH solution. The mixture was added to the resin and was agitated for 12 hours at room temperature. The slurry was drained and then the resin wash washed with 1.0 mL of methanol four times and 1.0 mL of DMF three times. The coupling was repeated a second time. Following complete coupling, A mixture of piperidine:DMF (20:80, 1 mL) was added to the resin and agitated for 10 to 15 minutes at room temperature. The piperidine solution was drained and then the resin was washed with 1.0 mL DMF three times.

Synthesis of 5. (Method: HATU) Fmoc-L-Lysine(Dde)-OH (KDde), CAS #150629-67-7, (4 equiv.), HATU (4 equiv.), and DIEA (8 equiv) in 1.0 mL of anhydrous NMP was prepared. The mixture was allowed to pre-activate at room temperature for 5 minutes, and then was added to the resin and agitated at 35° C. for 30 minutes. The mixture was drained and then the resin was washed with 1.0 mL of DMF three times. To remove Fmoc, A mixture of piperidine:DMF (20:80, 1 mL) was added to the resin and agitated for 15 minutes at room temperature. The piperidine solution was drained and then the resin was washed with 1.0 mL DMF three times.

Synthesis of 6. (Method: MITS) Three steps are required to mono-methylate the terminal amine. 1) Nosyl protection. 2,6-lutidine (6 equiv.) dissolved in 0.5 mL of anhydrous DCE was added to the resin. 2-nitrobenzenesulfonyl chloride (5 equiv.) dissolved in 0.5 mL anhydrous toluene was added to the resin then was agitated at 35° C. for 10 minutes. The mixture was drained and then the resin was washed with 1.0 mL of anhydrous toluene three times. The method was repeated twice. 2) Alkylation via mitsunobu conditions. Triphenylphosphine (10 equiv.) dissolved in 0.7 mL anhydrous toluene was added to the resin. Methanol (MeOH) (20 equiv.) was added to the resin. Azodicarboxylate (10 equiv) was added to the resin and was agitated at 45° C. for 30 minutes. The mixture was drained and then the resin was washed with 1.0 mL of anhydrous DMF three times. Alkylation was repeated twice. 3) Nosyl deprotection. 2-mercaptoethanol (5 equiv.) and 1,8-Diazabicyclo[5.4.0]undec-7-ene (5 equiv.) in 1.0 mL NMP was added to the resin and was agitated at 35° C. for 10 minutes. The mixture was drained and then the resin was washed with 1.0 mL of anhydrous DMF three times. Deprotection of the nosyl group was repeated twice.

Synthesis of 7. (Method: HATU) Fmoc-L-Cyclobutylalanine-OH (CycBuA), CAS #478183-62-9, (4 equiv.), HATU (4 equiv.), and DIEA (8 equiv) in 1.0 mL of anhydrous NMP was prepared. The mixture was allowed to pre-activate at room temperature for 5 minutes, and then was added to the resin and agitated at 35° C. to 45° C. for 10 to 90 minutes. The mixture was drained and then the resin was washed with 1.0 mL of DMF three times. The coupling step was repeated. To remove Fmoc, A mixture of piperidine:DMF (20:80, 1 mL) was added to the resin and agitated for 10 to 15 minutes at room temperature. The piperidine solution was drained and then the resin was washed with 1.0 mL DMF three times.

Synthesis of 8. (Method: HATU) Fmoc-(2S,4R)-4-fluoro-1,2-pyrrolidinecarboxylate (AA0011), CAS #203866-20-0, (4 equiv.), HATU (4 equiv.), and DIEA (8 equiv) in 1.0 mL of anhydrous NMP was prepared. The mixture was allowed to pre-activate at room temperature for 5 minutes, and then was added to the resin and agitated at 35° C. for 30 minutes. The mixture was drained and then the resin was washed with 1.0 mL of DMF three times. To remove Fmoc, A mixture of piperidine:DMF (20:80, 1 mL) was added to the resin and agitated for 10 to 15 minutes at room temperature. The piperidine solution was drained and then the resin was washed with 1.0 mL DMF three times.

Synthesis of 9. (Method: HATUnf) (2R)-3,3,3-trifluoro-2-hydroxy-2-methylpropanoic acid (Acd0486), CAS #44864-47-3, (4 equiv.), HATU (4 equiv.), and DIEA (8 equiv) in 1.0 mL of anhydrous NMP was prepared and adjusted to pH 9. The mixture added to the resin and agitated at 35° C. for 30 minutes. The mixture was drained and then the resin was washed with 1.0 mL of DMF three times. The coupling was repeated a second time.

Synthesis of 10. (Method: DdeR) To remove the Dde protecting group, 10% hydrazine monohydrate in in 1.0 mL NMP was added to the resin and was agitated for 20 minutes at room temperature. The mixture was drained and then the resin was washed with 1.0 mL DMF three times.

Synthesis of 11. (Method: 24% HFIP) To cleave peptide from CTC resin, approximately 2 mL of a solution of 24% HFIP, 2% TIPS, in DCM was added to the 100 mg of polystyrene resin in a solid phase reaction vessel. The contents were shaken for 1 hour. The cleavage solution was filtered into a 50 mL conical vial. The cleaved resin was washed with an additional 2 mL of DCM and the wash was collected in the conical vial. The solution was evaporated in a Genevac. The linear peptide was purified via reverse-phase HPLC using an Acetonitrile/Water gradient with 0.05% formic acid. The purified fractions were pooled and lyophilized to yield white powder (LCMS m/z observed=994.44 [M+H]⁺).

Synthesis of 12. (Method: PyBop Method B) The linear peptide (50 mg) was cyclized using a large volume, high dilution method. The linear peptide was transferred to a 500 mL round bottom flask with a stir bar, and dissolved in DCM (250 mL). DIEA (3 eqv) was added to the flask, followed by PyBop (3 eqv). The pH was adjusted to 9 with DIEA. The reaction was stirred at room temperature for 12 hours and monitored for reaction completion via LCMS (m/z observed=976.43 [M+Z]⁺).

### 7. Exemplary Compounds—Summary Tables

Table 2A and B, below, lists the Building Blocks and procedures used to prepare the listed exemplified compounds of Formula I, with stepwise transformations (T1-T18) listed left to right. The Building Blocks in Table 2A and B are listed using a Short Hand Name identified in Table 1. The procedures in Table 2A and B are listed using the abbreviations identified in the preceding general methods subheadings. For example, in Example 1, Transformation 1 (T1), building block ‘nva’ (Fmoc-D-norvaline) was coupled to 2-chlorotritylchloride resin via the “CTC” procedure.

If the column is empty, it means that this synthetic Transformation was not performed for this particular compound.

The following generally describes the function of each listed transformation:
Transformation 1 (T1): Attachment of Residue 9 to CTC Resin. See, for example, preparation of Example 456 in Section IX.B.6. of the current application; Table 2A/2B, Example 456; Section IX.B.1.i. “CTC-resin loading.”
Transformation 2 (T2): Alkylation of backbone nitrogen of Residue 9 (R^{9a}). See, for example, Table 2A/2B, Example 298; Section IX.B.1.xii. “MITS—Nosylation, mitsunobu, nosyl deprotection.”
Transformation 3 (T3): Peptide bond formation between Residue 8 and Residue 9. See, for example, preparation of Example 456 in Section IX.B.6. of the current application; Table 2A/2B, Example 456; Section IX.B.1.iii. “HATU—peptide coupling, followed by Fmoc deprotection.”
Transformation 4 (T4): Alkylation of backbone nitrogen of Residue 8 (R^{8a}). See, for example, preparation of Example 456 in Section IX.B.6. of the current application; Table 2A/2B, Example 456; Section IX.B.1.xii. “MITS—Nosylation, mitsunobu, nosyl deprotection.”
Transformation 5 (T5): Peptide bond formation between Residue 7 and Residue 8. See, for example, preparation of Example 456 in Section IX.B.6. of the current application; Table 2A/2B, Example 456; Section IX.B.1.vii. “DIC—sterically-hindered peptide coupling, followed by Fmoc deprotection.”
Transformation 6 (T6): Peptide bond formation between Residue 6 and Residue 7. See, for example, preparation of Example 456 in Section IX.B.6. of the current application; Table 2A/2B, Example 456; Section IX.B.1.iii. “HATU—peptide coupling, followed by Fmoc deprotection.”
Transformation 7 (T7): Alkylation of backbone nitrogen of Residue 6 (R^{6a}). See, for example, preparation of Example 456 in Section IX.B.6. of the current application; Table 2A/2B, Example 456; Section IX.B.1.xii. “MITS—Nosylation, mitsunobu, nosyl deprotection.”
Transformation 8 (T8): Peptide bond formation between Residue 5 and Residue 6. See, for example, preparation of Example 456 in Section IX.B.6. of the current application; Table 2A/2B, Example 456; Section IX.B.1.iii. “HATU—peptide coupling, followed by Fmoc deprotection.”
Transformation 9 (T9): Peptide bond formation between Residue 4 and Residue 5. See, for example, preparation of Example 456 in Section IX.B.6. of the current application; Table 2A/2B, Example 456; Section IX.B.1.iii. “HATU—peptide coupling, followed by Fmoc deprotection.”
Transformation 10 (T10): Peptide bond formation between Residue 3 and Residue 4. See, for example, preparation of Example 456 in Section IX.B.6. of the current application; Table 2A/2B, Example 456; Section IX.B.1.iv. “HATUnf—peptide coupling, no Fmoc deprotection.”
Transformation 11 (T11): Acylation of Residue 3. See, for example, Table 2A/2B, Example 461; Section IX.B.1.xiv. “Ac—acetylation of amines.”
Transformation 12 (T12): Deprotection of Dde group from the sidechain of Residue 4, 5, or 6. See, for example, preparation of Example 456 in Section IX.B.6. of the current application; Table 2A/2B, Example 456; Section IX.B.1.x. “DdeR—Dde removal via hydrazine”
Transformation 13 (T13): Introduction of alkyl or acyl group onto Sidechain of Residue 6 (R^{6d}). See, for example, Table 2A/2B, Example 496; Section IX.B.1.xii. “MITS—Nosylation, mitsunobu, nosyl deprotection.”
Transformation 14 (T14): Introduction of alkyl or acyl group onto Sidechain of Residue 5 (R5^{b/c}). See, for example, Table 2A/2B, Example 3; Section IX.B.1.xiii. “Morph—conversion of a primary amine to a morpholine moiety.”
Transformation 15 (T15): Introduction of alkyl or acyl group onto Sidechain of Residue 4 (R4^{b/c}). See, for example, Table 2A/2B, Example 9; Section IX.B.1.xiii. “Morph—conversion of a primary amine to a morpholine moiety.”
Transformation 16 (T16): Cleavage of linear peptide from solid phase resin. See, for example, preparation of Example 456 in Section IX.B.6. of the current application; Table 2A/2B, Example 456; Section IX.B.2.xviii “24% HFIP—resin cleavage.”
Transformation 17 (T17): Cyclization of sidechain amine to C-terminal carboxylic acid in solution. See, for example, preparation of Example 456 in Section IX.B.6. of the current application; Table 2A/2B, Example 456; Section IX.B.3.xxii “PyBop—cyclization in the presence of hydroxyl groups”
Transformation 18 (T18): Deprotection of remaining protecting groups in solution. See, for example, Table 2A/2B, Example 405; Section IX.B.3.xxiii “Solution Deprotection,” Boc.

TABLE 2A
Building Blocks used and Procedures for Compound Preparation (Part 1)

| Ex. | Type | T1 | T2 | T3 | T4 | T5 | T6 | T7 | T8 | T9 |
|---|---|---|---|---|---|---|---|---|---|---|
| 1 | Method | CTC | | HATU | RA | DIC | HATU | | DIC_KMe2 | DIC_KMe2 |
| | Building | nva | | 3CNF | Ald0003 | L | KMtt | | KMe2 | KMe2 |
| | Block | | | | | | | | | |
| 2 | Method | CTC | | HATU | MITS | HATU | HATU | MITS | HATU | HATU |
| | Building | nva | | 25ClF | MeOH | L | KBoc | MeOH | KAc | P |
| | Block | | | | | | | | | |
| 3 | Method | CTC | | HATU | MITS | HATU | HATU | MITS | HATU | HATU |
| | Building | nva | | 25ClF | MeOH | L | KMe | MeOH | KDde | Abu |
| | Block | | | | | | | | | |
| 4 | Method | CTC | | HATU | MITS | HATU | HATU | MITS | HATU | HATU |
| | Building | Abu | | 25ClF | MeOH | L | KBoc | MeOH | Abu | Abu |
| | Block | | | | | | | | | |
| 5 | Method | CTC | | HATU | MITS | DIC | HATU | MITS | HATU | HATU |
| | Building | nva | | 3ClF | Alc0046 | L | KMe | MeOH | KDde | Abu |
| | Block | | | | | | | | | |
| 6 | Method | CTC | | HATU | MITS | DIC | HATU | MITS | DIC_KMe2 | Onto_KMe2 |
| | Building | abu | | 3ClF | EtOH | L | KMe | MeOH | KMe2 | Abu |
| | Block | | | | | | | | | |
| 7 | Method | CTC | | HATU | MITS | DIC | HATU | MITS | HATU | HATU |
| | Building | a | | 3ClF | EtOH | L | KMe | MeOH | KDde | Abu |
| | Block | | | | | | | | | |
| 8 | Method | CTC | | HATU | MITS | HATU | HATU | MITS | HATU | HATU |
| | Building | nva | | 25ClF | MeOH | L | KBoc | MeOH | KAc | Abu |
| | Block | | | | | | | | | |
| 9 | Method | CTC | | HATU | MITS | HATU | HATU | MITS | HATU | HATU |
| | Building | nva | | 25ClF | MeOH | L | KMe | MeOH | KAc | KDde |
| | Block | | | | | | | | | |
| 10 | Method | CTC | | HATU | MITS | HATU | HATU | MITS | HATU | HATU |
| | Building | nva | | 25ClF | MeOH | L | KMe | MeOH | ODde | Abu |
| | Block | | | | | | | | | |
| 11 | Method | CTC | | HATU | MITS | HATU | HATU | MITS | HATU | HATU |
| | Building | ala | | 25ClF | MeOH | L | KMe | MeOH | KDde | Abu |
| | Block | | | | | | | | | |
| 12 | Method | CTC | | HATU | MITS | HATU | HATU | MITS | HATU | HATU |
| | Building | abu | | 25ClF | MeOH | L | KMe | MeOH | KDde | Abu |
| | Block | | | | | | | | | |
| 13 | Method | CTC | | HATU | MITS | DIC | HATU | MITS | HATU | HATU |
| | Building | nva | | 25ClF | EtOH | L | KMe | MeOH | KDde | Abu |
| | Block | | | | | | | | | |
| 14 | Method | CTC | | HATU | MITS | DIC | HATU | MITS | HATU | HATU |
| | Building | nva | | 25ClF | Alc0046 | L | KMe | MeOH | KDde | Abu |
| | Block | | | | | | | | | |
| 15 | Method | CTC | | HATU | MITS | DIC | HATU | MITS | HATU | HATU |
| | Building | nva | | 25ClF | Alc0046 | L | KBoc | MeOH | KDde | Abu |
| | Block | | | | | | | | | |
| 16 | Method | CTC | | HATU | MITS | HATU | HATU | MITS | HATU | HATU |
| | Building | nva | | 25ClF | MeOH | L | KMe | MeOH | KDde | P |
| | Block | | | | | | | | | |
| 17 | Method | CTC | | HATU | MITS | HATU | HATU | MITS | HATU | HATU |
| | Building | p | | 25ClF | MeOH | L | KMe | MeOH | KDde | Abu |
| | Block | | | | | | | | | |
| 18 | Method | CTC | | HATU | MITS | HATU | HATU | MITS | HATU | HATU |
| | Building | nva | | 25ClF | MeOH | L | KMe | MeOH | Abu | KDde |
| | Block | | | | | | | | | |
| 19 | Method | CTC | | HATU | MITS | DIC | HATU | MITS | HATU | HATU |
| | Building | nva | | 3ClF | Alc0046 | L | KMe | MeOH | A | KDde |
| | Block | | | | | | | | | |
| 20 | Method | CTC | | HATU | MITS | DIC | HATU | MITS | HATU | HATU |
| | Building | nva | | 3ClF | Alc0046 | L | KMe | MeOH | KDde | KDde |
| | Block | | | | | | | | | |
| 21 | Method | CTC | | HATU | MITS | HATU | HATU | MITS | DIC | HATU |
| | Building | nva | | 25ClF | MeOH | L | KMe | EtOH | KDde | Abu |
| | Block | | | | | | | | | |
| 22 | Method | CTC | | HATU | MITS | HATU | HATU | MITS | HATU | HATU |
| | Building | nva | | 25ClF | MeOH | TBA | KMe | MeOH | KDde | Abu |
| | Block | | | | | | | | | |
| 23 | Method | CTC | | HATU | MITS | HATU | HATU | MITS | HATU | HATU |
| | Building | nva | | 25ClF | MeOH | L | KMe | MeOH | KDde | Abu |
| | Block | | | | | | | | | |
| 24 | Method | CTC | | HATU | MITS | HATU | HATU | MITS | HATU | HATU |
| | Building | nva | | 25ClF | MeOH | L | KMe | MeOH | KDde | Abu |
| | Block | | | | | | | | | |
| 25 | Method | CTC | | HATU | MITS | HATU | HATU | MITS | DIC | HATU |
| | Building | nva | | 25ClF | MeOH | L | KBoc | EtOH | KDde | Abu |
| | Block | | | | | | | | | |
| 26 | Method | CTC | | HATU | MITS | HATU | HATU | MITS | DIC | HATU |
| | Building | nva | | 25ClF | MeOH | L | KBoc | Alc0050 | KDde | Abu |
| | Block | | | | | | | | | |
| 27 | Method | CTC | | HATU | MITS | HATU | HATU | MITS | HATU | HATU |
| | Building | nva | | 5Cl2OcPrF | MeOH | L | KBoc | MeOH | A | Abu |
| | Block | | | | | | | | | |
| 28 | Method | CTC | | HATU | MITS | DIC | HATU | MITS | HATU | HATU |
| | Building | a | | 5Cl2OcPrF | Alc0046 | L | KMe | MeOH | KAc | Abu |
| | Block | | | | | | | | | |
| 29 | Method | CTC | | HATU | MITS | HATU | HATU | MITS | HATU | HATU |
| | Building | nva | | 25ClF | MeOH | L | KBoc | MeOH | LysO | P |
| | Block | | | | | | | | | |
| 30 | Method | CTC | | HATU | MITS | HATU | HATU | MITS | HATU | HATU |
| | Building | a | | 25ClF | MeOH | L | KBoc | MeOH | A | P |
| | Block | | | | | | | | | |
| 31 | Method | CTC | | HATU | MITS | HATU | HATU | MITS | HATU | HATU |
| | Building | G | | 25ClF | MeOH | L | KBoc | MeOH | A | P |
| | Block | | | | | | | | | |
| 32 | Method | CTC | | HATU | MITS | DIC | HATU | MITS | HATU | HATU |
| | Building | nva | | 25ClF | Alc0046 | L | KBoc | MeOH | KAc | Abu |
| | Block | | | | | | | | | |
| 33 | Method | CTC | | HATU | MITS | DIC | HATU | MITS | HATU | HATU |
| | Building | sMe | | 25ClF | Alc0046 | L | KBoc | MeOH | KAc | Abu |
| | Block | | | | | | | | | |
| 34 | Method | CTC | | HATU | MITS | DIC | HATU | MITS | HATU | HATU |
| | Building | a | | 3ClF | Alc0045 | L | KMe | MeOH | KAc | Abu |
| | Block | | | | | | | | | |
| 35 | Method | CTC | | HATU | MITS | DIC | HATU | MITS | HATU | HATU |
| | Building | a | | 25ClF | Alc0046 | L | KMe | MeOH | KAc | Abu |
| | Block | | | | | | | | | |
| 36 | Method | CTC | | HATU | MITS | HATU | HATU | MITS | HATU | HATU |
| | Building | nva | | 25ClF | MeOH | L | KBoc | MeOH | KAc | P |
| | Block | | | | | | | | | |
| 37 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 38 | Method | CTC | | HATU | MITS | HATU | HATU | MITS | HATU | HATU |
| | Building | abu | | 25ClF | MeOH | L | KMe | MeOH | A | Abu |
| | Block | | | | | | | | | |
| 39 | Method | CTC | | HATU | MITS | HATU | HATU | MITS | HATU | HATU |
| | Building | abu | | 25ClF | MeOH | L | KMe | MeOH | A | Abu |
| | Block | | | | | | | | | |
| 40 | Method | CTC | | HATU | MITS | HATU | HATU | MITS | HATU | HATU |
| | Building | nva | | 25ClF | MeOH | L | KBoc | MeOH | KTFA | Abu |
| | Block | | | | | | | | | |
| 41 | Method | CTC | | HATU | MITS | HATU | HATU | MITS | HATU | HATU |
| | Building | nva | | 25ClF | MeOH | L | KBoc | MeOH | A | SHOP |
| | Block | | | | | | | | | |
| 42 | Method | CTC | | HATU | MITS | HATU | HATU | MITS | HATU | HATU |
| | Building | sMe | | 25ClF | MeOH | L | KBoc | MeOH | hSerTrt | P |
| | Block | | | | | | | | | |
| 43 | Method | CTC | | HATU | MITS | HATU | HATU | MITS | HATU | HATU |
| | Building | sMe | | 25ClF | MeOH | L | KBoc | MeOH | hSerTrt | P |
| | Block | | | | | | | | | |
| 44 | Method | CTC | | HATU | MITS | HATU | HATU | MITS | HATU | HATU |
| | Building | sMe | | 25ClF | MeOH | L | KMe | MeOH | hSerTrt | P |
| | Block | | | | | | | | | |
| 45 | Method | CTC | | HATU | MITS | HATU | HATU | MITS | HATU | HATU |
| | Building | sMe | | 25ClF | MeOH | L | KMe | MeOH | hSerTrt | P |
| | Block | | | | | | | | | |
| 46 | Method | CTC | | HATU | MITS | HATU | HATU | MITS | HATU | HATU |
| | Building | sMe | | 25ClF | MeOH | L | KBoc | MeOH | Abu | P |
| | Block | | | | | | | | | |
| 47 | Method | CTC | | HATU | MITS | HATU | HATU | MITS | HATU | HATU |
| | Building | a | | 25ClF | MeOH | L | KBoc | MeOH | Abu | P |
| | Block | | | | | | | | | |
| 48 | Method | CTC | | HATU | MITS | HATU | HATU | MITS | HATU | HATU |
| | Building | sMe | | 25ClF | MeOH | L | KMe | MeOH | Abu | P |
| | Block | | | | | | | | | |
| 49 | Method | CTC | | HATU | MITS | DIC | HATU | MITS | HATU | HATU |
| | Building | nva | | 3ClF | EtOH | L | KMe | MeOH | KAc | P |
| | Block | | | | | | | | | |
| 50 | Method | CTC | | HATU | MITS | DIC | HATU | MITS | HATU | HATU |
| | Building | a | | 5Cl2OcPrF | EtOH | L | KMe | MeOH | KAc | Abu |
| | Block | | | | | | | | | |
| 51 | Method | CTC | | HATU | MITS | HATU | HATU | MITS | HATU | HATU |
| | Building | Sar | | 25ClF | MeOH | L | KMe | MeOH | A | P |
| | Block | | | | | | | | | |
| 52 | Method | CTC | | HATU | MITS | DIC | HATU | MITS | HATU | HATU |
| | Building | sMe | | 25ClF | Alc0046 | L | KBoc | MeOH | A | P |
| | Block | | | | | | | | | |
| 53 | Method | CTC | | HATU | MITS | HATU | HATU | MITS | HATU | HATU |
| | Building | nva | | 5Cl2OcHexF | MeOH | L | KBoc | MeOH | A | Abu |
| | Block | | | | | | | | | |
| 54 | Method | CTC | | HATU | MITS | HATU | HATU | MITS | HATU | HATU |
| | Building | nva | | 5Cl2IF | MeOH | L | KBoc | MeOH | A | Abu |
| | Block | | | | | | | | | |
| 55 | Method | CTC | | HATU | MITS | HATU | HATU | MITS | HATU | HATU |
| | Building | nva | | 25ClF | MeOH | L | KBoc | MeOH | T | AA0011 |
| | Block | | | | | | | | | |
| 56 | Method | CTC | | HATU | MITS | HATU | HATU | MITS | HATU | HATU |
| | Building | abu | | 25ClF | MeOH | L | KMe | MeOH | A | P |
| | Block | | | | | | | | | |
| 57 | Method | CTC | | HATU | MITS | HATU | HATU | MITS | HATU | HATUnf |
| | Building | abu | | 25ClF | MeOH | L | KMe | MeOH | A | Acd0536 |
| | Block | | | | | | | | | |
| 58 | Method | CTC | | HATU | MITS | HATU | HATU | MITS | HATU | HATU |
| | Building | nva | | 5Cl2OcPrF | MeOH | L | KBoc | MeOH | A | Abu |
| | Block | | | | | | | | | |
| 59 | Method | CTC | | HATU | MITS | HATU | HATU | MITS | DIC | HATU |
| | Building | nva | | 25ClF | MeOH | L | KBoc | EtOH | KDde | Abu |
| | Block | | | | | | | | | |
| 60 | Method | CTC | | HATU | MITS | HATU | HATU | MITS | HATU | HATU |
| | Building | abu | | 25ClF | MeOH | L | KMe | MeOH | KDde | Abu |
| | Block | | | | | | | | | |
| 61 | Method | CTC | | HATU | MITS | HATU | HATU | MITS | HATU | HATU |
| | Building | abu | | 25ClF | MeOH | L | KMe | MeOH | KTFE | Abu |
| | Block | | | | | | | | | |
| 62 | Method | CTC | | HATU | MITS | HATU | HATU | MITS | HATU | HATU |
| | Building | abu | | 25ClF | MeOH | L | KBoc | MeOH | A | Abu |
| | Block | | | | | | | | | |
| 63 | Method | CTC | | HATU | MITS | HATU | HATU | MITS | HATU | HATU |
| | Building | aib | | 25ClF | MeOH | L | KBoc | MeOH | A | Abu |
| | Block | | | | | | | | | |
| 64 | Method | CTC | | HATU | MITS | HATU | HATU | MITS | HATU | HATU |
| | Building | sMe | | 25ClF | MeOH | L | KMe | MeOH | Abu | P |
| | Block | | | | | | | | | |
| 65 | Method | CTC | | HATU | MITS | HATU | HATU | MITS | HATU | HATU |
| | Building | sMe | | 25ClF | MeOH | L | KMe | MeOH | hSerMe | P |
| | Block | | | | | | | | | |
| 66 | Method | CTC | | HATU | MITS | HATU | HATU | MITS | HATU | HATU |
| | Building | sMe | | 25ClF | MeOH | L | KMe | MeOH | hSerTrt | P |
| | Block | | | | | | | | | |
| 67 | Method | CTC | | HATU | MITS | HATU | HATU | MITS | HATU | HATU |
| | Building | a | | 25ClF | MeOH | L | KMe | MeOH | A | Abu |
| | Block | | | | | | | | | |
| 68 | Method | CTC | | HATU | MITS | HATU | HATU | MITS | HATU | HATU |
| | Building | abu | | 25ClF | MeOH | L | KMe | MeOH | A | A |
| | Block | | | | | | | | | |
| 69 | Method | CTC | | HATU | MITS | HATU | HATU | MITS | HATU | HATU |
| | Building | cPrg | | 25ClF | MeOH | L | KMe | MeOH | A | Abu |
| | Block | | | | | | | | | |
| 70 | Method | CTC | | HATU | MITS | HATU | HATU | MITS | HATU | HATU |
| | Building | abu | | 25ClF | MeOH | L | KMe | MeOH | cPrG | Abu |
| | Block | | | | | | | | | |
| 71 | Method | CTC | | HATU | MITS | HATU | HATU | MITS | HATU | HATU |
| | Building | G | | 25ClF | MeOH | L | KBoc | MeOH | A | Abu |
| | Block | | | | | | | | | |
| 72 | Method | CTC | | HATU | MITS | HATU | HATU | MITS | HATU | HATUnf |
| | Building | G | | 25ClF | MeOH | L | KMe | MeOH | A | Acd0536 |
| | Block | | | | | | | | | |
| 73 | Method | CTC | | HATU | MITS | HATU | HATU | MITS | HATU | HATU |
| | Building | a | | 25ClF | MeOH | L | KBoc | MeOH | A | Pro |
| | Block | | | | | | | | | |
| 74 | Method | CTC | | HATU | MITS | HATU | HATU | MITS | HATU | HATU |
| | Building | G | | 25ClF | MeOH | L | KBoc | MeOH | A | P |
| | Block | | | | | | | | | |
| 75 | Method | CTC | | HATU | MITS | HATU | HATU | MITS | HATU | HATU |
| | Building | abu | | 25ClF | MeOH | L | KMe | MeOH | A | P |
| | Block | | | | | | | | | |
| 76 | Method | CTC | | HATU | MITS | HATU | HATU | MITS | HATU | HATU |
| | Building | Aib | | 25ClF | MeOH | L | KBoc | MeOH | A | P |
| | Block | | | | | | | | | |
| 77 | Method | CTC | | HATU | MITS | HATU | HATU | MITS | HATU | HATU |
| | Building | Aib | | 25ClF | MeOH | L | KBoc | MeOH | Abu | P |
| | Block | | | | | | | | | |
| 78 | Method | CTC | | HATU | MITS | HATU | HATU | MITS | HATU | HATU |
| | Building | abu | | 25ClF | MeOH | L | KMe | MeOH | G | P |
| | Block | | | | | | | | | |
| 79 | Method | CTC | | HATU | MITS | DIC | HATU | MITS | HATU | HATU |
| | Building | a | | 3ClF | EtOH | L | KMe | MeOH | Abu | P |
| | Block | | | | | | | | | |
| 80 | Method | CTC | | HATU | MITS | HATU | HATU | MITS | HATU | HATU |
| | Building | sMe | | 25ClF | MeOH | L | KMe | MeOH | A | P |
| | Block | | | | | | | | | |
| 81 | Method | CTC | | HATU | MITS | HATU | HATU | MITS | HATU | HATUnf |
| | Building | abu | | 5Cl2OMeF | MeOH | L | KMe | MeOH | A | Acd0536 |
| | Block | | | | | | | | | |
| 82 | Method | CTC | | HATU | MITS | HATU | HATU | MITS | DIC | HATU |
| | Building | a | | 25ClF | MeOH | L | KBoc | Alc0046 | A | P |
| | Block | | | | | | | | | |
| 83 | Method | CTC | | HATU | MITS | HATU | HATU | MITS | HATU | HATU |
| | Building | abu | | 25ClF | MeOH | L | KMe | MeOH | T | AA0011 |
| | Block | | | | | | | | | |
| 84 | Method | CTC | | HATU | MITS | HATU | HATU | MITS | HATU | HATU |
| | Building | abu | | 25ClF | MeOH | L | KMe | MeOH | A | P |
| | Block | | | | | | | | | |
| 85 | Method | CTC | | HATU | MITS | HATU | HATU | MITS | HATU | HATU |
| | Building | abu | | 25ClF | MeOH | L | KMe | MeOH | A | P |
| | Block | | | | | | | | | |
| 86 | Method | CTC | | HATU | MITS | HATU | HATU | MITS | HATU | HATU |
| | Building | abu | | 25ClF | MeOH | L | KMe | MeOH | A | 2Aze |
| | Block | | | | | | | | | |
| 87 | Method | CTC | | HATU | MITS | HATU | HATU | MITS | HATU | HATU |
| | Building | abu | | 25ClF | MeOH | CycBuA | KMe | MeOH | A | P |
| | Block | | | | | | | | | |
| 88 | Method | CTC | | HATU | MITS | HATU | HATU | MITS | HATU | HATU |
| | Building | abu | | 25ClF | MeOH | L | KMe | MeOH | A | P |
| | Block | | | | | | | | | |
| 89 | Method | CTC | | HATU | MITS | HATU | HATU | MITS | HATU | HATU |
| | Building | abu | | 25ClF | MeOH | L | KMe | MeOH | A | P |
| | Block | | | | | | | | | |
| 90 | Method | CTC | | HATU | MITS | HATU | HATU | MITS | HATU | HATU |
| | Building | Aib | | 25ClF | MeOH | L | KBoc | MeOH | A | AA0011 |
| | Block | | | | | | | | | |
| 91 | Method | CTC | | HATU | MITS | HATU | HATU | MITS | HATU | HATU |
| | Building | G | | 25ClF | MeOH | L | KBoc | MeOH | cPrG | Abu |
| | Block | | | | | | | | | |
| 92 | Method | CTC | | HATU | MITS | HATU | HATU | MITS | HATU | HATU |
| | Building | G | | 25ClF | MeOH | L | KBoc | MeOH | IcPrG | P |
| | Block | | | | | | | | | |
| 93 | Method | CTC | | HATU | MITS | HATU | HATU | MITS | HATU | HATU |
| | Building | G | | 25ClF | MeOH | L | KMe | MeOH | cPrG | Abu |
| | Block | | | | | | | | | |
| 94 | Method | CTC | | HATU | MITS | HATU | HATU | MITS | HATU | HATU |
| | Building | G | | 25ClF | MeOH | L | KMe | MeOH | cPrG | P |
| | Block | | | | | | | | | |
| 95 | Method | CTC | | HATU | MITS | HATU | HATU | MITS | HATU | HATU |
| | Building | abu | | 25ClF | MeOH | L | KMe | MeOH | CPA | P |
| | Block | | | | | | | | | |
| 96 | Method | CTC | | HATU | MITS | HATU | HATU | MITS | HATU | HATU |
| | Building | abu | | 25ClF | MeOH | L | KMe | MeOH | cPrG | P |
| | Block | | | | | | | | | |
| 97 | Method | CTC | | HATU | MITS | HATU | HATU | MITS | HATU | HATU |
| | Building | Aib | | 25ClF | MeOH | L | KBoc | MeOH | cPrG | Abu |
| | Block | | | | | | | | | |
| 98 | Method | CTC | | HATU | MITS | HATU | HATU | MITS | HATU | HATU |
| | Building | Aib | | 25ClF | MeOH | L | KBoc | MeOH | A | AA0011 |
| | Block | | | | | | | | | |
| 99 | Method | CTC | | HATU | MITS | HATU | HATU | MITS | HATU | HATUnf |
| | Building | abu | | 25ClF | MeOH | TBA | KMe | MeOH | A | Acd0536 |
| | Block | | | | | | | | | |
| 100 | Method | CTC | | HATU | MITS | HATU | HATU | MITS | HATU | HATU |
| | Building | abu | | 25ClF | MeOH | L | KMe | MeOH | cPrG | A |
| | Block | | | | | | | | | |
| 101 | Method | CTC | | HATU | MITS | HATU | HATU | MITS | DIC | HATU |
| | Building | abu | | 25ClF | MeOH | L | KBoc | Alc0050 | KDde | Abu |
| | Block | | | | | | | | | |
| 102 | Method | CTC | | HATU | MITS | HATU | HATU | MITS | HATU | HATU |
| | Building | abu | | 25ClF | MeOH | L | KBoc | MeOH | KDde | Abu |
| | Block | | | | | | | | | |
| 103 | Method | CTC | | HATU | MITS | HATU | HATU | MITS | DIC | HATU |
| | Building | sMe | | 25ClF | MeOH | L | KMe | EtOH | Abu | KDde |
| | Block | | | | | | | | | |
| 104 | Method | CTC | | HATU | MITS | HATU | HATU | MITS | DIC | HATUnf |
| | Building | sMe | | 25ClF | MeOH | L | KMe | EtOH | A | Acd0536 |
| | Block | | | | | | | | | |
| 105 | Method | CTC | | HATU | MITS | HATU | HATU | MITS | DIC | HATU |
| | Building | sMe | | 25ClF | MeOH | L | KMe | EtOH | cPrG | Abu |
| | Block | | | | | | | | | |
| 106 | Method | CTC | | HATU | MITS | HATU | HATU | MITS | DIC | HATUnf |
| | Building | nva | | 3ClF | MeOH | L | KBoc | EtOH | T | Acd0536 |
| | Block | | | | | | | | | |
| 107 | Method | CTC | | HATU | MITS | HATU | HATU | MITS | DIC | HATU |
| | Building | abu | | 25ClF | MeOH | L | KMe | EtOH | A | Abu |
| | Block | | | | | | | | | |
| 108 | Method | CTC | | HATU | MITS | HATU | HATU | MITS | DIC | HATUnf |
| | Building | abu | | 25ClF | MeOH | L | KMe | EtOH | A | Acd0536 |
| | Block | | | | | | | | | |
| 109 | Method | CTC | | HATU | MITS | HATU | HATU | MITS | DIC | HATU |
| | Building | abu | | 25ClF | MeOH | L | KMe | EtOH | cPrG | Abu |
| | Block | | | | | | | | | |
| 110 | Method | CTC | | HATU | MITS | HATU | HATU | MITS | DIC | HATU |
| | Building | Aib | | 25ClF | MeOH | L | KBoc | EtOH | A | Abu |
| | Block | | | | | | | | | |
| 111 | Method | CTC | | HATU | MITS | HATU | HATU | MITS | DIC | HATU |
| | Building | abu | | 3CNF | MeOH | L | KMe | EtOH | A | P |
| | Block | | | | | | | | | |
| 112 | Method | CTC | | HATU | MITS | HATU | HATU | MITS | HATU | HATU |
| | Building | Aib | | 25ClF | MeOH | L | KBoc | MeOH | Abu | 44FP |
| | Block | | | | | | | | | |
| 113 | Method | CTC | | HATU | MITS | HATU | HATU | MITS | HATU | HATU |
| | Building | a | | 25ClF | MeOH | L | KBoc | MeOH | Abu | 44FP |
| | Block | | | | | | | | | |
| 114 | Method | CTC | | HATU | MITS | HATU | HATU | MITS | HATU | HATUnf |
| | Building | a | | 25ClF | MeOH | L | KBoc | MeOH | Abu | Acd0574 |
| | Block | | | | | | | | | |
| 115 | Method | CTC | | HATU | MITS | HATU | HATU | MITS | HATU | HATU |
| | Building | Aib | | 25ClF | MeOH | L | KBoc | MeOH | Abu | Abu |
| | Block | | | | | | | | | |
| 116 | Method | CTC | | HATU | MITS | HATU | HATU | MITS | HATU | HATUnf |
| | Building | Aib | | 25ClF | MeOH | L | KBoc | MeOH | Abu | Acd0574 |
| | Block | | | | | | | | | |
| 117 | Method | CTC | | HATU | MITS | HATU | HATU | MITS | HATU | HATU |
| | Building | Aib | | 25ClF | MeOH | L | KBoc | MeOH | Abu | 44FP |
| | Block | | | | | | | | | |
| 118 | Method | CTC | | HATU | MITS | HATU | HATU | MITS | HATU | HATUnf |
| | Building | sMe | | 25ClF | MeOH | L | KMe | MeOH | hSerMe | Acd0574 |
| | Block | | | | | | | | | |
| 119 | Method | CTC | | HATU | MITS | HATU | HATU | MITS | HATU | HATUnf |
| | Building | abu | | 25ClF | MeOH | L | KMe | MeOH | A | Acd0574 |
| | Block | | | | | | | | | |
| 120 | Method | CTC | | HATU | MITS | HATU | HATU | MITS | HATU | HATU |
| | Building | abu | | 25ClF | MeOH | L | KMe | MeOH | A | 44FP |
| | Block | | | | | | | | | |
| 121 | Method | CTC | | HATU | MITS | HATU | HATU | | HATU | HATU |
| | Building | abu | | 25ClF | MeOH | L | KMe | | A | Abu |
| | Block | | | | | | | | | |
| 122 | Method | CTC | | HATU | MITS | HATU | HATU | MITs | HATU | HATU |
| | Building | abu | | 25ClF | MeOH | L | KDde | MeOH | KTFE | Abu |
| | Block | | | | | | | | | |
| 123 | Method | CTC | | HATU | MITS | HATU | HATU | MITS | HATU | HATU |
| | Building | Aib | | 25ClF | MeOH | L | KBoc | MeOH | Abu | Abu |
| | Block | | | | | | | | | |
| 124 | Method | CTC | | HATU | MITS | HATU | HATU | MITS | DIC | HATU |
| | Building | Aib | | 25ClF | MeOH | L | KBoc | EtOH | Abu | Abu |
| | Block | | | | | | | | | |
| 125 | Method | CTC | | HATU | MITS | HATU | HATU | | HATU | HATU |
| | Building | abu | | 25ClF | MeOH | L | KBoc | | A | Abu |
| | Block | | | | | | | | | |
| 126 | Method | CTC | | HATU | MITS | HATU | HATU | MITS | HATU | HATU |
| | Building | pro | | 25ClF | MeOH | L | KBoc | MeOH | A | Abu |
| | Block | | | | | | | | | |
| 127 | Method | CTC | | HATU | MITS | HATU | HATU | | HATU | HATU |
| | Building | abu | | 25ClF | MeOH | L | KMe | | KDde | Abu |
| | Block | | | | | | | | | |
| 128 | Method | CTC | | HATU | MITS | HATU | HATU | MITS | HATU | HATUnf |
| | Building | abu | | 25ClF | MeOH | L | KMe | MeOH | cPrG | Acd0574 |
| | Block | | | | | | | | | |
| 129 | Method | CTC | | HATU | MITS | HATU | HATU | MITS | HATU | HATUnf |
| | Building | Aib | | 25ClF | MeOH | L | KBoc | MeOH | A | Acd0574 |
| | Block | | | | | | | | | |
| 130 | Method | CTC | | HATU | MITS | HATU | HATU | MITS | HATU | HATU |
| | Building | a | | 3ClF | MeOH | L | KBoc | MeOH | Abu | 44FP |
| | Block | | | | | | | | | |
| 131 | Method | CTC | | HATU | MITS | HATU | HATU | MITS | HATU | HATU |
| | Building | Aib | | 3ClF | MeOH | L | KBoc | MeOH | Abu | 44FP |
| | Block | | | | | | | | | |
| 132 | Method | CTC | | HATU | MITS | HATU | HATU | MITS | HATU | HATU |
| | Building | Aib | | 25ClF | MeOH | L | KBoc | MeOH | hL | Abu |
| | Block | | | | | | | | | |
| 133 | Method | CTC | | HATU | MITS | HATU | HATU | MITS | HATU | HATU |
| | Building | abu | | 25ClF | MeOH | L | KMe | MeOH | DabDde | P |
| | Block | | | | | | | | | |
| 134 | Method | CTC | | HATU | MITS | HATU | HATU | MITS | HATU | HATUnf |
| | Building | abu | | 25ClF | MeOH | L | KMe | MeOH | DabDde | Acd0574 |
| | Block | | | | | | | | | |
| 135 | Method | CTC | | HATU | MITS | HATU | HATU | MITS | HATU | HATU |
| | Building | abu | | 25ClF | MeOH | L | KMe | MeOH | DabDde | Abu |
| | Block | | | | | | | | | |
| 136 | Method | CTC | | HATU | MITS | HATU | HATU | MITS | HATU | HATU |
| | Building | Aib | | 25ClF | MeOH | L | KBoc | MeOH | DabDde | Abu |
| | Block | | | | | | | | | |
| 137 | Method | CTC | | HATU | MITS | HATU | HATU | MITS | HATU | HATU |
| | Building | abu | | 25ClF | MeOH | L | KBoc | MeOH | A | P |
| | Block | | | | | | | | | |
| 138 | Method | CTC | | HATU | MITS | HATU | HATU | MITS | HATU | HATU |
| | Building | abu | | 25ClF | MeOH | L | KBoc | MeOH | cPrG | Abu |
| | Block | | | | | | | | | |
| 139 | Method | CTC | | HATU | MITS | HATU | HATU | MITS | HATU | HATU |
| | Building | abu | | 25ClF | MeOH | L | KBoc | MeOH | JA | P |
| | Block | | | | | | | | | |
| 140 | Method | CTC | | HATU | MITS | HATU | HATU | MITS | HATU | HATU |
| | Building | sMe | | 25ClF | MeOH | L | KBoc | MeOH | hSerMe | P |
| | Block | | | | | | | | | |
| 141 | Method | CTC | | HATU | MITS | HATU | HATU | MITS | HATU | HATU |
| | Building | pro | | 25ClF | MeOH | L | KBoc | MeOH | hSerMe | P |
| | Block | | | | | | | | | |
| 142 | Method | CTC | | HATU | MITS | HATU | HATU | MITS | HATU | HATU |
| | Building | sMe | | 25ClF | MeOH | L | KMe | MeOH | hSerMe | AA0011 |
| | Block | | | | | | | | | |
| 143 | Method | CTC | | HATU | MITS | HATU | HATU | MITS | HATU | HATU |
| | Building | a | | 25ClF | MeOH | L | KMe | MeOH | hSerMe | AA0011 |
| | Block | | | | | | | | | |
| 144 | Method | CTC | | HATU | MITS | HATU | HATU | MITS | HATU | HATU |
| | Building | nva | | 25ClF | MeOH | L | KMe | MeOH | hSerMe | AA0011 |
| | Block | | | | | | | | | |
| 145 | Method | CTC | | HATU | MITS | HATU | HATU | MITS | HATU | HATU |
| | Building | nva | | 25ClF | MeOH | L | KMe | MeOH | hSerMe | P |
| | Block | | | | | | | | | |
| 146 | Method | CTC | | HATU | MITS | HATU | HATU | MITS | HATU | HATU |
| | Building | sMe | | 25ClF | MeOH | L | KMe | MeOH | hSerMe | P |
| | Block | | | | | | | | | |
| 147 | Method | CTC | | HATU | MITS | HATU | HATU | MITS | HATU | HATU |
| | Building | sMe | | 25ClF | MeOH | L | KMe | MeOH | hSerMe | P |
| | Block | | | | | | | | | |
| 148 | Method | CTC | | HATU | MITS | HATU | HATU | MITS | DIC | HATU |
| | Building | a | | 25ClF | MeOH | L | KDde | Alc0046 | A | P |
| | Block | | | | | | | | | |
| 149 | Method | CTC | | HATU | MITS | HATU | HATU | MITS | DIC | HATU |
| | Building | a | | 25ClF | MeOH | L | KBoc | Alc0046 | A | P |
| | Block | | | | | | | | | |
| 150 | Method | CTC | | HATU | MITS | HATU | HATU | MITS | DIC | HATUnf |
| | Building | a | | 25ClF | MeOH | L | KBoc | Alc0046 | A | Acd0575 |
| | Block | | | | | | | | | |
| 151 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 152 | Method | CTC | | HATU | MITS | HATU | HATU | MITS | HATU | HATU |
| | Building | a | | 25ClF | MeOH | L | KBoc | MeOH | A | AA0011 |
| | Block | | | | | | | | | |
| 153 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 154 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 155 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 156 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 157 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 158 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 159 | Method | CTC | | HATU | MITS | HATU | HATU | MITS | HATU | HATU |
| | Building | a | | 25ClF | MeOH | L | KMe | MeOH | Abu | AA0011 |
| | Block | | | | | | | | | |
| 160 | Method | CTC | | HATU | MITS | HATU | HATU | MITS | HATU | HATU |
| | Building | G | | 25ClF | MeOH | L | KBoc | MeOH | hSerMe | Abu |
| | Block | | | | | | | | | |
| 161 | Method | CTC | | HATU | MITS | HATU | HATU | MITS | HATU | HATU |
| | Building | a | | 25ClF | MeOH | L | KBoc | MeOH | hSerMe | Abu |
| | Block | | | | | | | | | |
| 162 | Method | CTC | | HATU | MITS | HATU | HATU | MITS | HATU | HATU |
| | Building | nva | | 25ClF | MeOH | L | KBoc | MeOH | hSerMe | Abu |
| | Block | | | | | | | | | |
| 163 | Method | CTC | | HATU | MITS | HATU | HATU | MITS | HATU | HATU |
| | Building | abu | | 25ClF | MeOH | L | KMe | MeOH | A | AA0011 |
| | Block | | | | | | | | | |
| 164 | Method | CTC | | HATU | MITS | HATU | HATU | MITS | HATU | HATU |
| | Building | abu | | 25ClF | MeOH | L | KMe | MeOH | Abu | AA0011 |
| | Block | | | | | | | | | |
| 165 | Method | CTC | | HATU | MITS | HATU | HATU | MITS | DIC | HATU |
| | Building | a | | 25ClF | MeOH | L | KBoc | Alc0046 | hSerMe | P |
| | Block | | | | | | | | | |
| 166 | Method | CTC | | HATU | MITS | HATU | HATU | MITS | DIC | HATUnf |
| | Building | a | | 25ClF | MeOH | L | KBoc | Alc0046 | hSerMe | Acd0575 |
| | Block | | | | | | | | | |
| 167 | Method | CTC | | HATU | MITS | HATU | HATU | MITS | HATU | HATU |
| | Building | nva | | 25ClF | MeOH | L | KBoc | MeOH | hSerMe | Abu |
| | Block | | | | | | | | | |
| 168 | Method | CTC | | HATU | MITS | HATU | HATU | MITS | HATU | HATU |
| | Building | a | | 25ClF | MeOH | L | KBoc | MeOH | A | AA0011 |
| | Block | | | | | | | | | |
| 169 | Method | CTC | | HATU | MITS | HATU | HATU | MITS | HATU | HATU |
| | Building | abu | | 25ClF | MeOH | L | KBoc | MeOH | cPrG | A |
| | Block | | | | | | | | | |
| 170 | Method | CTC | | HATU | MITS | HATU | HATU | | HATU | HATU |
| | Building | abu | | 25ClF | MeOH | L | KMe | | cPrG | A |
| | Block | | | | | | | | | |
| 171 | Method | CTC | | HATU | MITS | HATU | HATU | MITS | HATU | HATU |
| | Building | abu | | 25ClF | MeOH | L | KBoc | MeOH | cPrG | A |
| | Block | | | | | | | | | |
| 172 | Method | CTC | | HATU | MITS | HATU | HATU | MITS | HATU | HATU |
| | Building | Aib | | 25ClF | MeOH | L | KBoc | MeOH | cPrG | A |
| | Block | | | | | | | | | |
| 173 | Method | CTC | | HATU | MITS | HATU | HATU | MITS | HATU | HATU |
| | Building | Aib | | 25ClF | MeOH | L | KBoc | MeOH | cPrG | A |
| | Block | | | | | | | | | |
| 174 | Method | CTC | | HATU | MITS | HATU | HATU | MITS | HATU | HATU |
| | Building | a | | 25ClF | MeOH | L | KBoc | MeOH | A | AA0011 |
| | Block | | | | | | | | | |
| 175 | Method | CTC | | HATU | MITS | HATU | HATU | MITS | HATU | HATU |
| | Building | abu | | 25ClF | MeOH | L | KMe | MeOH | A | P |
| | Block | | | | | | | | | |
| 176 | Method | CTC | | HATU | MITS | HATU | HATU | MITS | HATU | HATU |
| | Building | Aib | | 25ClF | MeOH | L | KBoc | MeOH | A | Abu |
| | Block | | | | | | | | | |
| 177 | Method | CTC | | HATU | MITS | HATU | HATU | MITS | HATU | HATU |
| | Building | Aib | | 25ClF | MeOH | L | KBoc | MeOH | Nva | Abu |
| | Block | | | | | | | | | |
| 178 | Method | CTC | | HATU | MITS | HATU | HATU | MITS | HATU | HATU |
| | Building | Aib | | 25ClF | MeOH | L | KBoc | MeOH | NL | Abu |
| | Block | | | | | | | | | |
| 179 | Method | CTC | | HATU | MITS | HATU | HATU | MITS | HATU | HATU |
| | Building | Aib | | 25ClF | MeOH | L | KBoc | MeOH | L | Abu |
| | Block | | | | | | | | | |
| 180 | Method | CTC | | HATU | MITS | HATU | HATU | MITS | HATU | HATU |
| | Building | Aib | | 25ClF | MeOH | L | KBoc | MeOH | CPA | Abu |
| | Block | | | | | | | | | |
| 181 | Method | CTC | | HATU | MITS | HATU | HATU | MITS | HATU | HATU |
| | Building | Aib | | 25ClF | MeOH | L | KBoc | MeOH | CycBuA | Abu |
| | Block | | | | | | | | | |
| 182 | Method | CTC | | HATU | MITS | HATU | HATU | MITS | DIC_KMe2 | Onto_KMe2 |
| | Building | Aib | | 25ClF | MeOH | L | KBoc | MeOH | KMe2 | Abu |
| | Block | | | | | | | | | |
| 183 | Method | CTC | | HATU | MITS | HATU | HATU | MITS | HATU | HATU |
| | Building | Aib | | 25ClF | MeOH | L | KBoc | MeOH | KDde | Abu |
| | Block | | | | | | | | | |
| 184 | Method | CTC | | HATU | MITS | HATU | HATU | MITS | HATU | HATU |
| | Building | Aib | | 25ClF | MeOH | L | KBoc | MeOH | T | Abu |
| | Block | | | | | | | | | |
| 185 | Method | CTC | | HATU | MITS | EEDQ | HATU | MITS | HATU | HATU |
| | Building | Aib | | 25ClF | Alc0046 | L | KBoc | MeOH | cPrG | A |
| | Block | | | | | | | | | |
| 186 | Method | CTC | | HATU | MITS | DIC | HATU | MITS | HATU | HATU |
| | Building | Aib | | 3ClF | EtOH | L | KBoc | MeOH | A | AA0011 |
| | Block | | | | | | | | | |
| 187 | Method | CTC | | HATU | MITS | DIC | HATU | MITS | HATU | HATU |
| | Building | Aib | | 25ClF | EtOH | L | KBoc | MeOH | Abu | AA0011 |
| | Block | | | | | | | | | |
| 188 | Method | CTC | | HATU | MITS | DIC | HATU | MITS | HATU | HATU |
| | Building | Aib | | 25ClF | Alc0046 | L | KBoc | MeOH | A | AA0011 |
| | Block | | | | | | | | | |
| 189 | Method | CTC | | HATU | MITS | DIC | HATU | MITS | HATU | HATU |
| | Building | a | | 5Cl2IF | EtOH | L | KMe | MeOH | Abu | Abu |
| | Block | | | | | | | | | |
| 190 | Method | CTC | | HATU | MITS | HATU | HATU | MITS | HATU | HATU |
| | Building | abu | | 3CNF | MeOH | L | KMe | MeOH | A | P |
| | Block | | | | | | | | | |
| 191 | Method | CTC | | HATU | MITS | HATU | HATU | MITS | HATU | HATU |
| | Building | Aib | | 25ClF | MeOH | L | KBoc | MeOH | Abu | AA0011 |
| | Block | | | | | | | | | |
| 192 | Method | CTC | | HATU | MITS | HATU | HATU | MITS | HATU | HATU |
| | Building | a | | 25ClF | MeOH | L | KBoc | MeOH | A | AA0011 |
| | Block | | | | | | | | | |
| 193 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 194 | Method | CTC | | HATU | MITS | HATU | HATU | MITS | HATU | HATU |
| | Building | Aib | | 25ClF | MeOH | L | KBoc | MeOH | Abu | AA0011 |
| | Block | | | | | | | | | |
| 195 | Method | CTC | | HATU | MITS | HATU | HATU | MITS | HATU | HATU |
| | Building | sMe | | 25ClF | MeOH | L | KMe | MeOH | Abu | AA0011 |
| | Block | | | | | | | | | |
| 196 | Method | CTC | | HATU | MITS | HATU | HATU | MITS | HATU | HATU |
| | Building | a | | 25ClF | MeOH | L | KMe | MeOH | Abu | AA0011 |
| | Block | | | | | | | | | |
| 197 | Method | CTC | | HATU | MITS | HATU | HATU | MITS | HATU | HATU |
| | Building | a | | 25ClF | MeOH | L | KMe | MeOH | Abu | AA0011 |
| | Block | | | | | | | | | |
| 198 | Method | CTC | | HATU | MITS | HATU | HATU | | HATU | HATUnf |
| | Building | Aib | | F | MeOH | L | KBoc | | Abu | Acd0575 |
| | Block | | | | | | | | | |
| 199 | Method | CTC | | HATU | MITS | HATU | HATU | MITS | HATU | HATUnf |
| | Building | Aib | | 25ClF | MeOH | L | KBoc | MeOH | A | Acd0575 |
| | Block | | | | | | | | | |
| 200 | Method | CTC | | HATU | MITS | HATU | HATU | MITS | HATU | HATU |
| | Building | a | | 3ClF | MeOH | L | KBoc | MeOH | Abu | AA0011 |
| | Block | | | | | | | | | |
| 201 | Method | CTC | | HATU | MITS | HATU | HATU | MITS | HATU | HATU |
| | Building | Aib | | 3ClF | MeOH | L | KBoc | MeOH | Abu | AA0011 |
| | Block | | | | | | | | | |
| 202 | Method | CTC | | HATU | MITS | DIC | HATU | MITS | HATU | HATU |
| | Building | a | | 3ClF | EtOH | L | KMe | MeOH | DabDde | AA0011 |
| | Block | | | | | | | | | |
| 203 | Method | CTC | | HATU | MITS | HATU | HATU | MITS | HATU | HATU |
| | Building | a | | 25ClF | MeOH | L | KBoc | MeOH | Abu | 44FP |
| | Block | | | | | | | | | |
| 204 | Method | CTC | | HATU | MITS | HATU | HATU | MITS | HATU | HATU |
| | Building | abu | | 25ClF | MeOH | L | KBoc | MeOH | T | AA0011 |
| | Block | | | | | | | | | |
| 205 | Method | CTC | | HATU | MITS | HATU | HATU | MITS | HATU | HATU |
| | Building | nva | | 25ClF | MeOH | L | KBoc | MeOH | T | AA0011 |
| | Block | | | | | | | | | |
| 206 | Method | CTC | | HATU | MITS | HATU | HATU | MITS | HATU | HATU |
| | Building | p | | 25ClF | MeOH | L | KBoc | MeOH | cPrG | AA0011 |
| | Block | | | | | | | | | |
| 207 | Method | CTC | | HATU | MITS | HATU | HATU | MITS | HATU | HATU |
| | Building | p | | 25ClF | MeOH | L | KBoc | MeOH | A | AA0011 |
| | Block | | | | | | | | | |
| 208 | Method | CTC | | HATU | MITS | HATU | HATU | MITS | DIC | HATU |
| | Building | sMe | | 25ClF | MeOH | L | KMe | EtOH | hSerMe | P |
| | Block | | | | | | | | | |
| 209 | Method | CTC | | HATU | MITS | HATU | HATU | MITS | DIC | HATU |
| | Building | p | | 25ClF | MeOH | L | KBoc | EtOH | A | Abu |
| | Block | | | | | | | | | |
| 210 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 211 | Method | CTC | | HATU | MITS | HATU | HATU | MITS | EEDQ | HATU |
| | Building | Aib | | 25ClF | MeOH | L | KBoc | EtOH | A | AA0011 |
| | Block | | | | | | | | | |
| 212 | Method | CTC | | HATU | MITS | HATU | HATU | MITS | EEDQ | HATU |
| | Building | Aib | | 25ClF | MeOH | L | KBoc | EtOH | Abu | AA0011 |
| | Block | | | | | | | | | |
| 213 | Method | CTC | | HATU | MITS | HATU | HATU | MITS | HATU | HATU |
| | Building | Aib | | 25ClF | MeOH | L | KBoc | MeOH | Abu | Abu |
| | Block | | | | | | | | | |
| 214 | Method | CTC | | HATU | MITS | HATU | HATU | MITS | HATU | HATU |
| | Building | Aib | | 25ClF | MeOH | L | KBoc | MeOH | Abu | Abu |
| | Block | | | | | | | | | |
| 215 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 216 | Method | CTC | | HATU | MITS | HATU | HATU | MITS | HATU | HATU |
| | Building | abu | | 25ClF | MeOH | L | KDde | MeOH | A | Abu |
| | Block | | | | | | | | | |
| 217 | Method | CTC | | HATU | MITS | HATU | HATU | MITS | HATU | HATU |
| | Building | abu | | 25ClF | MeOH | L | KMe | CD3OD | A | Abu |
| | Block | | | | | | | | | |
| 218 | Method | CTC | | HATU | MITS | HATU | HATU | MITS | HATU | HATU |
| | Building | abu | | 25ClF | CD3OD | L | KDde | CD3OD | A | Abu |
| | Block | | | | | | | | | |
| 219 | Method | CTC | | HATU | MITS | HATU | HATU | MITS | HATU | HATU |
| | Building | Aib | | 25ClF | MeOH | L | KBoc | MeOH | A | AA0011 |
| | Block | | | | | | | | | |
| 220 | Method | CTC | | HATU | MITS | HATU | HATU | MITS | HATU | HATU |
| | Building | Aib | | 25ClF | MeOH | L | KBoc | MeOH | A | AA0011 |
| | Block | | | | | | | | | |
| 221 | Method | CTC | | HATU | MITS | HATU | HATU | MITS | HATU | HATU |
| | Building | Aib | | 25ClF | MeOH | L | KBoc | MeOH | cPrG | A |
| | Block | | | | | | | | | |
| 222 | Method | CTC | | HATU | MITS | HATU | HATU | MITS | HATU | HATU |
| | Building | Aib | | 25ClF | MeOH | L | KBoc | MeOH | cPrG | A |
| | Block | | | | | | | | | |
| 223 | Method | CTC | | HATU | MITS | HATU | HATU | MITS | HATU | HATU |
| | Building | Aib | | 25ClF | MeOH | L | KBoc | MeOH | cPrG | A |
| | Block | | | | | | | | | |
| 224 | Method | CTC | | HATU | MITS | HATU | HATU | MITS | HATU | HATU |
| | Building | p | | 25ClF | MeOH | L | KBoc | MeOH | A | AA0011 |
| | Block | | | | | | | | | |
| 225 | Method | CTC | | HATU | MITS | HATU | HATU | MITS | HATU | HATU |
| | Building | p | | 25ClF | MeOH | L | KBoc | MeOH | A | AA0011 |
| | Block | | | | | | | | | |
| 226 | Method | CTC | | HATU | MITS | HATU | HATU | MITS | HATU | HATU |
| | Building | p | | 25ClF | MeOH | L | KBoc | MeOH | A | AA0011 |
| | Block | | | | | | | | | |
| 227 | Method | CTC | | HATU | MITS | HATU | HATU | MITS | HATU | HATU |
| | Building | p | | 25ClF | MeOH | L | KBoc | MeOH | A | AA0011 |
| | Block | | | | | | | | | |
| 228 | Method | CTC | | HATU | MITS | HATU | HATU | MITS | HATU | HATU |
| | Building | p | | 25ClF | MeOH | L | KBoc | MeOH | A | AA0011 |
| | Block | | | | | | | | | |
| 229 | Method | CTC | | HATU | MITS | HATU | HATU | MITS | HATU | HATU |
| | Building | p | | 25ClF | MeOH | L | KBoc | MeOH | A | AA0011 |
| | Block | | | | | | | | | |
| 230 | Method | CTC | | HATU | MITS | HATU | HATU | MITS | HATU | HATU |
| | Building | sMe | | 25ClF | MeOH | L | KMe | MeOH | hSerMe | P |
| | Block | | | | | | | | | |
| 231 | Method | CTC | | HATU | MITS | HATU | HATU | MITS | HATU | HATU |
| | Building | sMe | | 25ClF | MeOH | L | KMe | MeOH | hSerMe | P |
| | Block | | | | | | | | | |
| 232 | Method | CTC | | HATU | MITS | HATU | HATU | MITS | HATU | HATU |
| | Building | sMe | | 25ClF | MeOH | L | KMe | MeOH | hSerMe | P |
| | Block | | | | | | | | | |
| 233 | Method | CTC | | HATU | MITS | HATU | HATU | MITS | EEDQ | HATU |
| | Building | Aib | | 25ClF | MeOH | L | KBoc | EtOH | Abu | Abu |
| | Block | | | | | | | | | |
| 234 | Method | CTC | | HATU | MITS | HATU | HATU | MITS | EEDQ | HATU |
| | Building | Aib | | 25ClF | MeOH | L | KBoc | EtOH | Abu | Abu |
| | Block | | | | | | | | | |
| 235 | Method | CTC | | HATU | MITS | HATU | HATU | MITS | EEDQ | HATU |
| | Building | Aib | | 25ClF | MeOH | L | KBoc | EtOH | Abu | Abu |
| | Block | | | | | | | | | |
| 236 | Method | CTC | | HATU | MITS | HATU | HATU | MITS | EEDQ | HATU |
| | Building | Aib | | 25ClF | MeOH | L | KBoc | EtOH | Abu | Abu |
| | Block | | | | | | | | | |
| 237 | Method | CTC | | HATU | MITS | HATU | HATU | MITS | DIC | HATU |
| | Building | Aib | | 25ClF | MeOH | L | KBoc | EtOH | Abu | AA0011 |
| | Block | | | | | | | | | |
| 238 | Method | CTC | | HATU | MITS | HATU | HATU | MITS | EEDQ | HATU |
| | Building | Aib | | 25ClF | MeOH | L | KBoc | EtOH | Abu | AA0011 |
| | Block | | | | | | | | | |
| 239 | Method | CTC | | HATU | MITS | HATU | HATU | MITS | DIC | HATU |
| | Building | Aib | | 25ClF | MeOH | L | KBoc | EtOH | Abu | AA0011 |
| | Block | | | | | | | | | |
| 240 | Method | CTC | | HATU | MITS | HATU | HATU | MITS | HATU | HATU |
| | Building | sMe | | 25ClF | MeOH | L | KBoc | MeOH | hSerMe | AA0011 |
| | Block | | | | | | | | | |
| 241 | Method | CTC | | HATU | MITS | HATU | HATU | MITS | DIC | HATU |
| | Building | sMe | | 25ClF | MeOH | L | KBoc | EtOH | hSerMe | AA0011 |
| | Block | | | | | | | | | |
| 242 | Method | CTC | | HATU | MITS | EEDQ | HATU | MITS | HATU | HATU |
| | Building | sMe | | 25ClF | Alc0046 | L | KMe | MeOH | hSerMe | AA0011 |
| | Block | | | | | | | | | |
| 243 | Method | CTC | | HATU | MITS | HATU | HATU | MITS | HATU | HATU |
| | Building | sMe | | 3ClF | MeOH | L | KMe | MeOH | hSerMe | AA0011 |
| | Block | | | | | | | | | |
| 244 | Method | CTC | | HATU | MITS | EEDQ | HATU | MITS | HATU | HATU |
| | Building | sMe | | 3ClF | Alc0046 | L | KMe | MeOH | hSerMe | AA0011 |
| | Block | | | | | | | | | |
| 245 | Method | CTC | | HATU | MITS | HATU | HATU | MITS | HATU | HATU |
| | Building | sMe | | 25ClF | MeOH | L | KMe | MeOH | hSerMe | AA0011 |
| | Block | | | | | | | | | |
| 246 | Method | CTC | | HATU | MITS | HATU | HATU | MITS | HATU | HATU |
| | Building | sMe | | 25ClF | MeOH | L | KBoc | MeOH | hSerMe | AA0011 |
| | Block | | | | | | | | | |
| 247 | Method | CTC | | HATU | MITS | DIC | HATU | MITS | HATU | HATU |
| | Building | sMe | | 25ClF | Alc0046 | L | KBoc | MeOH | hSerMe | AA0011 |
| | Block | | | | | | | | | |
| 248 | Method | CTC | | HATU | MITS | HATU | HATU | MITS | HATU | HATU |
| | Building | a | | 25ClF | MeOH | L | KBoc | MeOH | hSerMe | AA0011 |
| | Block | | | | | | | | | |
| 249 | Method | CTC | | HATU | MITS | HATU | HATU | MITS | HATU | HATU |
| | Building | a | | 25ClF | MeOH | L | KMe | MeOH | hSerMe | P |
| | Block | | | | | | | | | |
| 250 | Method | CTC | | HATU | MITS | HATU | HATU | MITS | HATU | HATU |
| | Building | Aib | | 25ClF | MeOH | L | KBoc | MeOH | hSerMe | AA0011 |
| | Block | | | | | | | | | |
| 251 | Method | CTC | | HATU | MITS | HATU | HATU | MITS | HATU | HATU |
| | Building | Aib | | 25ClF | MeOH | L | KBoc | MeOH | Nva | AA0011 |
| | Block | | | | | | | | | |
| 252 | Method | CTC | | HATU | MITS | HATU | HATU | MITS | DIC | HATU |
| | Building | Aib | | 25ClF | MeOH | L | KBoc | EtOH | A | AA0011 |
| | Block | | | | | | | | | |
| 253 | Method | CTC | | HATU | MITS | HATU | HATU | MITS | EEDQ | HATUnf |
| | Building | Aib | | 3CNF | MeOH | L | KBoc | EtOH | A | Acd0574 |
| | Block | | | | | | | | | |
| 254 | Method | CTC | | HATU | MITS | HATU | HATU | MITS | DIC | HATUnf |
| | Building | p | | 3CNF | MeOH | L | KBoc | EtOH | A | Acd0574 |
| | Block | | | | | | | | | |
| 255 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 256 | Method | CTC | | HATU | MITS | HATU | HATU | MITS | HATU | HATU |
| | Building | a | | 25ClF | MeOH | L | KBoc | MeOH | Abu | AA0011 |
| | Block | | | | | | | | | |
| 257 | Method | CTC | | HATU | MITS | HATU | HATU | MITS | DIC | HATUnf |
| | Building | Aib | | 25ClF | MeOH | L | KBoc | EtOH | A | Acd0575 |
| | Block | | | | | | | | | |
| 258 | Method | CTC | | HATU | MITS | HATU | HATU | MITS | DIC | HATU |
| | Building | Aib | | 25ClF | MeOH | L | KBoc | EtOH | A | AA0011 |
| | Block | | | | | | | | | |
| 259 | Method | CTC | | HATU | MITS | HATU | HATU | MITS | EEDQ | HATU |
| | Building | p | | 25ClF | MeOH | L | KBoc | EtOH | A | AA0011 |
| | Block | | | | | | | | | |
| 260 | Method | CTC | | HATU | MITS | HATU | HATU | MITS | HATU | HATU |
| | Building | a | | 25ClF | MeOH | L | KBoc | MeOH | Nva | AA0011 |
| | Block | | | | | | | | | |
| 261 | Method | CTC | | HATU | MITS | HATU | HATU | MITS | HATU | HATU |
| | Building | nva | | 25ClF | MeOH | L | KBoc | MeOH | Abu | AA0011 |
| | Block | | | | | | | | | |
| 262 | Method | CTC | | HATU | MITS | DIC | HATU | MITS | HATU | HATU |
| | Building | aMeabu | | 25ClF | MeOH | L | KBoc | MeOH | A | AA0011 |
| | Block | | | | | | | | | |
| 263 | Method | CTC | | HATU | MITS | HATU | HATU | MITS | HATU | HATU |
| | Building | CVa | | 25ClF | MeOH | L | KBoc | MeOH | A | AA0011 |
| | Block | | | | | | | | | |
| 264 | Method | CTC | | HATU | MITS | HATU | HATU | MITS | HATU | HATU |
| | Building | a | | 5Cl2IF | MeOH | L | KBoc | MeOH | Abu | AA0011 |
| | Block | | | | | | | | | |
| 265 | Method | CTC | | HATU | MITS | HATU | HATU | MITS | HATU | HATU |
| | Building | p | | 5Cl2IF | MeOH | L | KBoc | MeOH | A | Abu |
| | Block | | | | | | | | | |
| 266 | Method | CTC | | HATU | MITS | HATU | HATU | MITS | HATU | HATU |
| | Building | Aib | | 5Cl2IF | MeOH | L | KBoc | MeOH | A | AA0011 |
| | Block | | | | | | | | | |
| 267 | Method | CTC | | HATU | MITS | HATU | HATU | MITS | HATU | HATU |
| | Building | nva | | 5Cl2OMePen | MeOH | L | KBoc | MeOH | A | Abu |
| | Block | | | | | | | | | |
| 268 | Method | CTC | | HATU | MITS | HATU | HATU | MITS | HATU | HATU |
| | Building | nva | | 25ClF | MeOH | L | KBoc | MeOH | A | AA0011 |
| | Block | | | | | | | | | |
| 269 | Method | CTC | | HATU | MITS | HATU | HATU | MITS | HATU | HATU |
| | Building | a | | 25ClF | MeOH | L | KMe | MeOH | A | AA0011 |
| | Block | | | | | | | | | |
| 270 | Method | CTC | | HATU | MITS | HATU | HATU | MITS | HATU | HATU |
| | Building | abu | | 25ClF | MeOH | L | KBoc | MeOH | A | AA0011 |
| | Block | | | | | | | | | |
| 271 | Method | CTC | | HATU | MITS | HATU | HATU | MITS | HATU | HATU |
| | Building | p | | 25ClF | MeOH | L | KMe | MeOH | hSerTrt | AA0011 |
| | Block | | | | | | | | | |
| 272 | Method | CTC | | HATU | MITS | HATU | HATU | MITS | HATU | HATU |
| | Building | p | | 25ClF | MeOH | L | KMe | MeOH | hSerTrt | AA0011 |
| | Block | | | | | | | | | |
| 273 | Method | CTC | | HATU | MITS | HATU | HATU | MITS | HATU | HATU |
| | Building | p | | 25ClF | MeOH | L | KBoc | MeOH | hSerMe | AA0011 |
| | Block | | | | | | | | | |
| 274 | Method | CTC | | HATU | MITS | HATU | HATU | MITS | HATU | HATU |
| | Building | sMe | | 25ClF | MeOH | L | KBoc | MeOH | hSerMe | AA0011 |
| | Block | | | | | | | | | |
| 275 | Method | CTC | | HATU | MITS | HATU | HATU | MITS | HATU | HATU |
| | Building | a | | 25ClF | MeOH | L | KBoc | MeOH | hSerMe | AA0011 |
| | Block | | | | | | | | | |
| 276 | Method | CTC | | HATU | MITS | HATU | HATU | MITS | HATU | HATU |
| | Building | p | | 25ClF | MeOH | L | KBoc | MeOH | hSerMe | AA0011 |
| | Block | | | | | | | | | |
| 277 | Method | CTC | | HATU | MITS | HATU | HATU | MITS | HATU | HATU |
| | Building | a | | 25ClF | CD3OD | L | KBoc | CD3OD | ACF3 | AA0011 |
| | Block | | | | | | | | | |
| 278 | Method | CTC | | HATU | MITS | HATU | HATU | MITS | HATU | HATU |
| | Building | p | | 25ClF | MeOH | L | KBoc | MeOH | ACF3 | AA0011 |
| | Block | | | | | | | | | |
| 279 | Method | CTC | | HATU | MITS | HATU | HATU | MITS | DIC | HATU |
| | Building | a | | 3ClF | MeOH | L | KBoc | EtOH | Abu | AA0011 |
| | Block | | | | | | | | | |
| 280 | Method | CTC | | HATU | MITS | HATU | HATU | MITS | DIC | HATU |
| | Building | abu | | 3ClF | MeOH | L | KBoc | EtOH | Abu | AA0011 |
| | Block | | | | | | | | | |
| 281 | Method | CTC | | HATU | MITS | HATU | HATU | MITS | HATU | HATU |
| | Building | p | | 3ClF | MeOH | L | KBoc | MeOH | Abu | AA0011 |
| | Block | | | | | | | | | |
| 282 | Method | CTC | | HATU | MITS | HATU | HATU | MITS | HATU | HATU |
| | Building | a | | 3ClF | MeOH | L | KMe | MeOH | Abu | AA0011 |
| | Block | | | | | | | | | |
| 283 | Method | CTC | | HATU | MITS | HATU | HATU | MITS | HATU | HATU |
| | Building | sMe | | 25ClF | MeOH | L | KMe | MeOH | cPrG | P |
| | Block | | | | | | | | | |
| 284 | Method | CTC | | HATU | MITS | HATU | HATU | MITS | HATU | HATU |
| | Building | sMe | | 25ClF | MeOH | L | KMe | MeOH | CPA | P |
| | Block | | | | | | | | | |
| 285 | Method | CTC | | HATU | MITS | HATU | HATU | MITS | HATU | HATU |
| | Building | sMe | | 25ClF | MeOH | L | KMe | MeOH | KDde | P |
| | Block | | | | | | | | | |
| 286 | Method | CTC | | HATU | MITS | HATU | HATU | MITS | HATU | HATU |
| | Building | pip | | 25ClF | MeOH | L | KBoc | MeOH | hSerTrt | P |
| | Block | | | | | | | | | |
| 287 | Method | CTC | | HATU | MITS | HATU | HATU | MITS | HATU | HATU |
| | Building | Acpc | | 25ClF | MeOH | L | KBoc | MeOH | Abu | P |
| | Block | | | | | | | | | |
| 288 | Method | CTC | | HATU | MITS | HATU | HATU | MITS | HATU | HATU |
| | Building | Acpc | | 25ClF | MeOH | L | KBoc | MeOH | Abu | AA0011 |
| | Block | | | | | | | | | |
| 289 | Method | CTC | | HATU | MITS | HATU | HATU | MITS | HATU | HATU |
| | Building | cFp | | 25ClF | MeOH | L | KBoc | MeOH | Abu | AA0011 |
| | Block | | | | | | | | | |
| 290 | Method | CTC | | HATU | MITS | HATU | HATU | MITS | HATU | HATU |
| | Building | cFp | | 25ClF | MeOH | L | KBoc | MeOH | A | AA0011 |
| | Block | | | | | | | | | |
| 291 | Method | CTC | | HATU | MITS | HATU | HATU | MITS | HATU | HATU |
| | Building | p | | 25ClF | MeOH | L | KBoc | MeOH | Abu | AA0011 |
| | Block | | | | | | | | | |
| 292 | Method | CTC | | HATU | MITS | HATU | HATU | MITS | HATU | HATU |
| | Building | p | | 25ClF | MeOH | L | KBoc | MeOH | Abu | AA0011 |
| | Block | | | | | | | | | |
| 293 | Method | CTC | | HATU | MITS | HATU | HATU | MITS | HATU | HATU |
| | Building | p | | 25ClF | MeOH | L | KBoc | MeOH | Abu | AA0011 |
| | Block | | | | | | | | | |
| 294 | Method | CTC | | HATU | MITS | HATU | HATU | MITS | DIC | HATU |
| | Building | p | | 25ClF | MeOH | L | KBoc | EtOH | Abu | AA0011 |
| | Block | | | | | | | | | |
| 295 | Method | CTC | | HATU | MITS | HATU | HATU | MITS | DIC | HATU |
| | Building | p | | 25ClF | MeOH | L | KBoc | EtOH | hSerMe | AA0011 |
| | Block | | | | | | | | | |
| 296 | Method | CTC | | HATU | MITS | HATU | HATU | | HATU | HATU |
| | Building | a | | 25ClF | MeOH | L | KBoc | | Abu | AA0011 |
| | Block | | | | | | | | | |
| 297 | Method | CTC | | HATU | MITS | HATU | HATU | | HATU | HATU |
| | Building | a | | 25ClF | MeOH | L | KMe | | Abu | AA0011 |
| | Block | | | | | | | | | |
| 298 | Method | CTC | | HATU | MITS | HATU | HATU | | HATU | HATU |
| | Building | a | MeOH | 25ClF | MeOH | L | KBoc | | Abu | AA0011 |
| | Block | | | | | | | | | |
| 299 | Method | CTC | | HATU | MITS | DIC | HATU | | HATU | HATU |
| | Building | a | | 25ClF | Alc0046 | L | KBoc | | Abu | AA0011 |
| | Block | | | | | | | | | |
| 300 | Method | CTC | | HATU | MITS | HATU | HATU | | HATU | HATU |
| | Building | nva | | 25ClF | MeOH | L | KBoc | | Abu | AA0011 |
| | Block | | | | | | | | | |
| 301 | Method | CTC | | HATU | MITS | HATU | HATU | | HATU | HATU |
| | Building | a | | 25ClF | MeOH | L | KBoc | | hL | AA0011 |
| | Block | | | | | | | | | |
| 302 | Method | CTC | | HATU | MITS | HATU | HATU | | HATU | HATU |
| | Building | a | | 25ClF | MeOH | L | KBoc | | Tle | AA0011 |
| | Block | | | | | | | | | |
| 303 | Method | CTC | | HATU | MITS | HATU | HATU | | HATU | HATUnf |
| | Building | a | | 25ClF | MeOH | L | KBoc | | Abu | Acd0575 |
| | Block | | | | | | | | | |
| 304 | Method | CTC | | HATU | MITS | HATU | HATU | | HATU | HATU |
| | Building | a | | 25ClF | MeOH | L | KBoc | | Abu | AA0011 |
| | Block | | | | | | | | | |
| 305 | Method | CTC | | HATU | MITS | HATU | HATU | MITS | HATU | HATU |
| | Building | a | | 2H45ClF | MeOH | L | KBoc | MeOH | Abu | AA0011 |
| | Block | | | | | | | | | |
| 306 | Method | CTC | | HATU | MITS | HATU | HATU | | HATU | HATU |
| | Building | a | | 2H55ClF | MeOH | L | NMeK | | Abu | AA0011 |
| | Block | | | | | | | | | |
| 307 | Method | CTC | | HATU | MITS | HATU | HATU | MITS | HATU | HATU |
| | Building | a | | 2H75ClF | MeOH | L | KBoc | MeOH | Abu | AA0011 |
| | Block | | | | | | | | | |
| 308 | Method | CTC | | HATU | MITS | HATU | HATU | MITS | HATU | HATU |
| | Building | a | | 25ClF | MeOH | L | KMe | MeOH | ODde | Abu |
| | Block | | | | | | | | | |
| 309 | Method | CTC | | HATU | MITS | DIC | HATU | MITS | HATU | HATU |
| | Building | a | | 3ClF | EtOH | L | KMe | MeOH | ODde | AA0011 |
| | Block | | | | | | | | | |
| 310 | Method | CTC | | HATU | MITS | HATU | HATU | MITS | HATU | HATU |
| | Building | a | | 25ClF | MeOH | L | KBoc | MeOH | ODde | AA0011 |
| | Block | | | | | | | | | |
| 311 | Method | CTC | | HATU | MITS | DIC | HATU | MITS | HATU | HATU |
| | Building | nva | | 3ClF | EtOH | L | KMe | MeOH | ODde | AA0011 |
| | Block | | | | | | | | | |
| 312 | Method | CTC | | HATU | MITS | HATU | HATU | MITS | HATU | HATU |
| | Building | nva | | 25ClF | MeOH | L | KBoc | MeOH | ODde | AA0011 |
| | Block | | | | | | | | | |
| 313 | Method | CTC | | HATU | MITS | HATU | HATU | MITS | HATU | HATU |
| | Building | Aib | | 3ClF | MeOH | L | KBoc | MeOH | hSerMe | AA0011 |
| | Block | | | | | | | | | |
| 314 | Method | CTC | | HATU | MITS | HATU | HATU | MITS | HATU | HATU |
| | Building | Aib | | 25ClF | MeOH | L | KBoc | MeOH | hSerMe | AA0011 |
| | Block | | | | | | | | | |
| 315 | Method | CTC | | HATU | MITS | HATU | HATU | MITS | HATU | HATU |
| | Building | p | | 25ClF | MeOH | L | KBoc | MeOH | hSerMe | AA0011 |
| | Block | | | | | | | | | |
| 316 | Method | CTC | | HATU | MITS | HATU | HATU | MITS | HATU | HATU |
| | Building | cFp | | 25ClF | MeOH | L | KBoc | MeOH | A | AA0011 |
| | Block | | | | | | | | | |
| 317 | Method | CTC | | HATU | MITS | HATU | HATU | MITS | HATU | HATU |
| | Building | p | | 25ClF | MeOH | L | KBoc | MeOH | A | AA0011 |
| | Block | | | | | | | | | |
| 318 | Method | CTC | | HATU | MITS | HATU | HATU | MITS | HATU | HATU |
| | Building | p | | 25ClF | MeOH | L | KBoc | MeOH | A | AA0011 |
| | Block | | | | | | | | | |
| 319 | Method | CTC | | HATU | MITS | DIC | HATU | | HATU | HATU |
| | Building | nva | | 25ClF | Alc0046 | L | KBoc | | Abu | AA0011 |
| | Block | | | | | | | | | |
| 320 | Method | CTC | | HATU | MITS | HATU | HATU | | HATU | HATU |
| | Building | a | | 25ClF | MeOH | L | KBoc | | Abu | AA0011 |
| | Block | | | | | | | | | |
| 321 | Method | CTC | | HATU | MITS | HATU | HATU | | HATU | HATU |
| | Building | p | | 25ClF | MeOH | L | KBoc | | Abu | AA0011 |
| | Block | | | | | | | | | |
| 322 | Method | CTC | | HATU | MITS | DIC | HATU | MITS | HATU | HATU |
| | Building | Aib | | 3ClF | Alc0046 | L | KBoc | MeOH | Abu | AA0011 |
| | Block | | | | | | | | | |
| 323 | Method | CTC | | HATU | MITS | DIC | HATU | MITS | HATU | HATU |
| | Building | CFFB | | 25ClF | MeOH | L | KBoc | MeOH | A | AA0011 |
| | Block | | | | | | | | | |
| 324 | Method | CTC | | HATU | MITS | HATU | HATU | MITS | HATU | HATU |
| | Building | Acpc | | 25ClF | MeOH | L | KBoc | MeOH | A | AA0011 |
| | Block | | | | | | | | | |
| 325 | Method | CTC | | HATU | MITS | HATU | HATU | | HATU | HATU |
| | Building | a | | 25ClF | MeOH | L | KiPr | | Abu | AA0011 |
| | Block | | | | | | | | | |
| 326 | Method | CTC | | HATU | MITS | HATU | HATU | MITS | HATU | HATU |
| | Building | cFp | | 25ClF | MeOH | L | KBoc | MeOH | Abu | AA0011 |
| | Block | | | | | | | | | |
| 327 | Method | CTC | | HATU | MITS | HATU | HATU | MITS | HATU | HATU |
| | Building | abu | | 25ClF | MeOH | L | KBoc | MeOH | KTFE | Abu |
| | Block | | | | | | | | | |
| 328 | Method | CTC | | HATU | MITS | HATU | HATU | MITS | HATU | HATU |
| | Building | p | | 25ClF | MeOH | L | KBoc | MeOH | KTFE | Abu |
| | Block | | | | | | | | | |
| 329 | Method | CTC | | HATU | MITS | HATU | HATU | MITS | HATU | HATU |
| | Building | Aib | | 25ClF | MeOH | L | KBoc | MeOH | cPrG | A |
| | Block | | | | | | | | | |
| 330 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 331 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 332 | Method | CTC | | HATU | MITS | HATU | HATU | MITS | HATU | HATU |
| | Building | Aib | | 25ClF | MeOH | L | KBoc | MeOH | CPA | AA0011 |
| | Block | | | | | | | | | |
| 333 | Method | CTC | | HATU | MITS | DIC | HATU | MITS | HATU | HATU |
| | Building | Aib | | 25ClF | MeOH | L | KBoc | MeOH | CBG | AA0011 |
| | Block | | | | | | | | | |
| 334 | Method | CTC | | HATU | MITS | HATU | HATU | MITS | HATU | HATU |
| | Building | Aib | | 25ClF | MeOH | L | KBoc | MeOH | CycBuA | AA0011 |
| | Block | | | | | | | | | |
| 335 | Method | CTC | | HATU | MITS | HATU | HATU | MITS | HATU | HATU |
| | Building | Acpc | | 25ClF | MeOH | L | KBoc | MeOH | cPrG | AA0011 |
| | Block | | | | | | | | | |
| 336 | Method | CTC | | HATU | MITS | HATU | HATU | MITS | HATU | HATU |
| | Building | Acpc | | 25ClF | MeOH | L | KBoc | MeOH | CPA | AA0011 |
| | Block | | | | | | | | | |
| 337 | Method | CTC | | HATU | MITS | DIC | HATU | MITS | HATU | HATU |
| | Building | Acpc | | 25ClF | MeOH | L | KBoc | MeOH | CBG | AA0011 |
| | Block | | | | | | | | | |
| 338 | Method | CTC | | HATU | MITS | HATU | HATU | MITS | HATU | HATU |
| | Building | Acpc | | 25ClF | MeOH | L | KBoc | MeOH | CycBuA | AA0011 |
| | Block | | | | | | | | | |
| 339 | Method | CTC | | HATU | MITS | HATU | HATU | MITS | HATU | HATU |
| | Building | cFp | | 25ClF | MeOH | L | KBoc | MeOH | cPrG | AA0011 |
| | Block | | | | | | | | | |
| 340 | Method | CTC | | HATU | MITS | HATU | HATU | MITS | HATU | HATU |
| | Building | cFp | | 25ClF | MeOH | L | KBoc | MeOH | CPA | AA0011 |
| | Block | | | | | | | | | |
| 341 | Method | CTC | | HATU | MITS | DIC | HATU | MITS | HATU | HATU |
| | Building | cFp | | 25ClF | MeOH | L | KBoc | MeOH | CBG | AA0011 |
| | Block | | | | | | | | | |
| 342 | Method | CTC | | HATU | MITS | HATU | HATU | MITS | HATU | HATU |
| | Building | cFp | | 25ClF | MeOH | L | KBoc | MeOH | A | AA0011 |
| | Block | | | | | | | | | |
| 343 | Method | CTC | | HATU | MITS | HATU | HATU | MITS | HATU | HATU |
| | Building | cFp | | 25ClF | MeOH | L | KBoc | MeOH | cPrG | AA0011 |
| | Block | | | | | | | | | |
| 344 | Method | CTC | | HATU | MITS | HATU | HATU | MITS | HATU | HATU |
| | Building | cFp | | 25ClF | MeOH | L | KBoc | MeOH | cPrG | AA0011 |
| | Block | | | | | | | | | |
| 345 | Method | CTC | | HATU | MITS | HATU | HATU | MITS | HATU | HATU |
| | Building | Acpc | | 25ClF | MeOH | L | KBoc | MeOH | Abu | AA0011 |
| | Block | | | | | | | | | |
| 346 | Method | CTC | | HATU | MITS | HATU | HATU | MITS | HATU | HATU |
| | Building | Acpc | | 25ClF | MeOH | L | KBoc | MeOH | cPrG | AA0011 |
| | Block | | | | | | | | | |
| 347 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 348 | Method | CTC | | HATU | MITS | HATU | HATU | MITS | HATU | HATU |
| | Building | cFp | | 25ClF | MeOH | L | KBoc | MeOH | cPrG | AA0011 |
| | Block | | | | | | | | | |
| 349 | Method | CTC | | HATU | MITS | HATU | HATU | MITS | HATU | HATU |
| | Building | p | | 25ClF | MeOH | L | KBoc | MeOH | cPrG | Abu |
| | Block | | | | | | | | | |
| 350 | Method | CTC | | HATU | MITS | HATU | HATU | MITS | HATU | HATU |
| | Building | cFp | | 25ClF | MeOH | L | KBoc | MeOH | cPrG | AA0011 |
| | Block | | | | | | | | | |
| 351 | Method | CTC | | HATU | MITS | HATU | HATU | MITS | HATU | HATU |
| | Building | p | | 25ClF | MeOH | L | KBoc | MeOH | cPrG | Abu |
| | Block | | | | | | | | | |
| 352 | Method | CTC | | HATU | MITS | HATU | HATU | MITS | HATU | HATU |
| | Building | p | | 25ClF | MeOH | L | KBoc | MeOH | cPrG | Abu |
| | Block | | | | | | | | | |
| 353 | Method | CTC | | HATU | | HATU | HATU | MITS | HATU | HATU |
| | Building | Aib | | NMe25ClF | | L | KBoc | MeOH | cPrG | AA0011 |
| | Block | | | | | | | | | |
| 354 | Method | CTC | | HATU | MITS | HATU | HATU | MITS | HATU | HATU |
| | Building | Gaba | | 25ClF | MeOH | L | DabDde | MeOH | cPrG | AA0011 |
| | Block | | | | | | | | | |
| 355 | Method | CTC | | HATU | MITS | HATU | HATU | MITS | HATU | HATU |
| | Building | Aib | | 2Me5ClF | MeOH | L | KDde | MeOH | A | AA0011 |
| | Block | | | | | | | | | |
| 356 | Method | CTC | | HATU | MITS | HATU | HATU | MITS | HATU | HATU |
| | Building | p | | 5Br2ClF | MeOH | L | KBoc | MeOH | Abu | AA0011 |
| | Block | | | | | | | | | |
| 357 | Method | CTC | | HATU | MITS | HATU | HATU | MITS | HATU | HATU |
| | Building | cFp | | 5Br2ClF | MeOH | L | KBoc | MeOH | Abu | AA0011 |
| | Block | | | | | | | | | |
| 358 | Method | CTC | | HATU | MITS | HATU | HATU | MITS | HATU | HATU |
| | Building | p | | 2F5ClF | MeOH | L | KBoc | MeOH | Abu | AA0011 |
| | Block | | | | | | | | | |
| 359 | Method | CTC | | HATU | MITS | HATU | HATU | MITS | HATU | HATU |
| | Building | p | | 5Br2ClF | MeOH | L | KBoc | MeOH | Abu | AA0011 |
| | Block | | | | | | | | | |
| 360 | Method | CTC | | HATU | MITS | HATU | HATU | MITS | HATU | HATU |
| | Building | cFp | | 5Br2ClF | MeOH | L | KBoc | MeOH | Abu | AA0011 |
| | Block | | | | | | | | | |
| 361 | Method | CTC | | HATU | MITS | HATU | HATU | MITS | HATU | HATU |
| | Building | Aib | | 2F5ClF | MeOH | L | KBoc | MeOH | cPrG | AA0011 |
| | Block | | | | | | | | | |
| 362 | Method | CTC | | HATU | MITS | HATU | HATU | MITS | HATU | HATU |
| | Building | Aib | | 2Cl5FF | MeOH | L | KBoc | MeOH | cPrG | AA0011 |
| | Block | | | | | | | | | |
| 363 | Method | CTC | | HATU | MITS | HATU | HATU | MITS | HATU | HATU |
| | Building | a | | 2Cl5FF | MeOH | L | KBoc | MeOH | A | AA0011 |
| | Block | | | | | | | | | |
| 364 | Method | CTC | | HATU | MITS | HATU | HATU | MITS | HATU | HATU |
| | Building | Acpc | | 2F5ClF | MeOH | L | KBoc | MeOH | Abu | AA0011 |
| | Block | | | | | | | | | |
| 365 | Method | CTC | | HATU | MITS | HATU | HATU | MITS | HATU | HATU |
| | Building | Acpc | | 2Cl5FF | MeOH | L | KBoc | MeOH | Abu | AA0011 |
| | Block | | | | | | | | | |
| 366 | Method | CTC | | HATU | MITS | HATU | HATU | MITS | HATU | HATU |
| | Building | Aib | | 2Cl5FF | MeOH | L | KBoc | MeOH | A | AA0011 |
| | Block | | | | | | | | | |
| 367 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 368 | Method | CTC | | HATU | MITS | HATU | HATU | MITS | HATU | HATU |
| | Building | Aib | | 25ClF | MeOH | L | KBoc | MeOH | A | AA0011 |
| | Block | | | | | | | | | |
| 369 | Method | CTC | | HATU | MITS | HATU | HATU | MITS | HATU | HATU |
| | Building | Acpc | | 25ClF | MeOH | L | KBoc | MeOH | cPrG | AA0011 |
| | Block | | | | | | | | | |
| 370 | Method | CTC | | HATU | MITS | HATU | HATU | MITS | HATU | HATU |
| | Building | Acpc | | 25ClF | MeOH | L | KBoc | MeOH | A | AA0011 |
| | Block | | | | | | | | | |
| 371 | Method | CTC | | HATU | MITS | HATU | HATU | MITS | HATU | HATU |
| | Building | a | | 3ClF | MeOH | L | KBoc | MeOH | Abu | AA0011 |
| | Block | | | | | | | | | |
| 372 | Method | CTC | | HATU | MITS | HATU | HATU | MITS | HATU | HATU |
| | Building | cFp | | 25ClF | MeOH | L | KBoc | MeOH | V | AA0011 |
| | Block | | | | | | | | | |
| 373 | Method | CTC | | HATU | MITS | HATU | HATU | MITS | HATU | HATU |
| | Building | cFp | | 25ClF | MeOH | L | KBoc | MeOH | CBG | Abu |
| | Block | | | | | | | | | |
| 374 | Method | CTC | | HATU | MITS | HATU | HATU | MITS | HATU | HATU |
| | Building | a | | 25ClF | MeOH | L | KBoc | MeOH | DabDde | AA0011 |
| | Block | | | | | | | | | |
| 375 | Method | CTC | | HATU | MITS | HATU | HATU | MITS | HATU | HATU |
| | Building | a | | 25ClF | MeOH | L | KBoc | MeOH | KDde | AA0011 |
| | Block | | | | | | | | | |
| 376 | Method | CTC | | HATU | MITS | HATU | HATU | MITS | HATU | HATU |
| | Building | a | | 25ClF | MeOH | L | KBoc | MeOH | ODde | AA0011 |
| | Block | | | | | | | | | |
| 377 | Method | CTC | | HATU | MITS | HATU | HATU | MITS | HATU | HATU |
| | Building | a | | 25ClF | MeOH | L | KBoc | MeOH | ODde | AA0011 |
| | Block | | | | | | | | | |
| 378 | Method | CTC | | HATU | MITS | HATU | HATU | MITS | HATU | HATU |
| | Building | nva | | 25ClF | MeOH | L | KBoc | MeOH | ODde | AA0011 |
| | Block | | | | | | | | | |
| 379 | Method | CTC | | HATU | MITS | HATU | HATU | MITS | HATU | HATU |
| | Building | cFp | | 25ClF | MeOH | L | KBoc | MeOH | cPrG | AA0011 |
| | Block | | | | | | | | | |
| 380 | Method | CTC | | HATU | MITS | HATU | HATU | MITS | HATU | HATU |
| | Building | cFp | | 2F5ClF | MeOH | L | KBoc | MeOH | cPrG | AA0011 |
| | Block | | | | | | | | | |
| 381 | Method | CTC | | HATU | MITS | HATU | HATU | MITS | HATU | HATU |
| | Building | cFp | | 2F5ClF | MeOH | L | KBoc | MeOH | cPrG | AA0011 |
| | Block | | | | | | | | | |
| 382 | Method | CTC | | HATU | MITS | DIC | HATU | MITS | HATU | HATU |
| | Building | cFp | | 25ClF | EtOH | L | KBoc | MeOH | cPrG | AA0011 |
| | Block | | | | | | | | | |
| 383 | Method | CTC | | HATU | MITS | DIC | HATU | | HATU | HATU |
| | Building | nva | | 3ClF | EtOH | L | KMe | | ODde | AA0011 |
| | Block | | | | | | | | | |
| 384 | Method | CTC | | HATU | MITS | HATU | HATU | | HATU | HATU |
| | Building | a | | 25ClF | MeOH | L | KMe | | ODde | AA0011 |
| | Block | | | | | | | | | |
| 385 | Method | CTC | | HATU | MITS | HATU | HATU | | HATU | HATU |
| | Building | a | | 25ClF | MeOH | L | KMe | | ODde | AA0011 |
| | Block | | | | | | | | | |
| 386 | Method | CTC | | HATU | MITS | DIC | HATU | MITS | HATU | HATU |
| | Building | nva | | 25ClF | EtOH | L | KBoc | MeOH | ODde | AA0011 |
| | Block | | | | | | | | | |
| 387 | Method | CTC | | HATU | MITS | HATU | HATU | MITS | HATU | HATU |
| | Building | nva | | 3ClF | MeOH | L | KMe | MeOH | ODde | AA0011 |
| | Block | | | | | | | | | |
| 388 | Method | CTC | | HATU | MITS | DIC | HATU | MITS | HATU | HATU |
| | Building | nva | | 25ClF | EtOH | L | KMe | MeOH | ODde | AA0011 |
| | Block | | | | | | | | | |
| 389 | Method | CTC | | HATU | MITS | HATU | HATU | MITS | HATU | HATU |
| | Building | Aib | | 3ClF | MeOH | L | KBoc | MeOH | cPrG | AA0011 |
| | Block | | | | | | | | | |
| 390 | Method | CTC | | HATU | MITS | DIC | HATU | MITS | HATU | HATU |
| | Building | Aib | | 3ClF | EtOH | L | KBoc | MeOH | cPrG | AA0011 |
| | Block | | | | | | | | | |
| 391 | Method | CTC | | HATU | MITS | DIC | HATU | MITS | HATU | HATU |
| | Building | Aib | | 25ClF | EtOH | L | KBoc | MeOH | cPrG | AA0011 |
| | Block | | | | | | | | | |
| 392 | Method | CTC | | HATU | MITS | DIC | HATU | | HATU | HATU |
| | Building | nva | | 3ClF | EtOH | L | KMe | | Nva | AA0011 |
| | Block | | | | | | | | | |
| 393 | Method | CTC | | HATU | MITS | HATU | HATU | | HATU | HATU |
| | Building | nva | | 25ClF | MeOH | L | KMe | | Nva | AA0011 |
| | Block | | | | | | | | | |
| 394 | Method | CTC | | HATU | MITS | DIC | HATU | MITS | HATU | HATU |
| | Building | a | | 20Me5ClF | EtOH | L | KMe | MeOH | Abu | Abu |
| | Block | | | | | | | | | |
| 395 | Method | CTC | | HATU | MITS | DIC | HATU | MITS | HATU | HATU |
| | Building | a | | 5Cl2OcPrF | EtOH | L | KMe | MeOH | Abu | Abu |
| | Block | | | | | | | | | |
| 396 | Method | CTC | | HATU | MITS | HATU | HATU | MITS | HATU | HATU |
| | Building | a | | 25ClF | MeOH | L | KBoc | MeOH | Abu | V |
| | Block | | | | | | | | | |
| 397 | Method | CTC | | HATU | MITS | HATU | HATU | MITS | HATU | HATU |
| | Building | a | | 25ClF | MeOH | L | KBoc | MeOH | Abu | KDde |
| | Block | | | | | | | | | |
| 398 | Method | CTC | | HATU | MITS | HATU | HATU | MITS | HATU | HATU |
| | Building | a | | 25ClF | MeOH | L | KBoc | MeOH | ODde | AA0011 |
| | Block | | | | | | | | | |
| 399 | Method | CTC | | HATU | MITS | HATU | HATU | MITS | HATU | HATU |
| | Building | a | | 25ClF | MeOH | L | KMe | MeOH | ODde | Abu |
| | Block | | | | | | | | | |
| 400 | Method | CTC | | HATU | MITS | HATU | HATU | MITS | HATU | HATU |
| | Building | Aib | | 3FF | MeOH | L | KDde | MeOH | cPrG | AA0011 |
| | Block | | | | | | | | | |
| 401 | Method | CTC | | HATU | MITS | HATU | HATU | MITS | HATU | HATU |
| | Building | cFp | | 2Cl5FF | MeOH | L | KBoc | MeOH | cPrG | AA0011 |
| | Block | | | | | | | | | |
| 402 | Method | CTC | | HATU | MITS | HATU | HATU | MITS | HATU | HATU |
| | Building | cFp | | 2Cl5FF | MeOH | L | KDde | MeOH | cPrG | AA0011 |
| | Block | | | | | | | | | |
| 403 | Method | CTC | | HATU | MITS | HATU | HATU | MITS | HATU | HATU |
| | Building | cFp | | 3ClF | MeOH | L | KBoc | MeOH | cPrG | AA0011 |
| | Block | | | | | | | | | |
| 404 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 405 | Method | CTC | | HATU | MITS | HATU | HATU | MITS | HATU | HATU |
| | Building | Aib | | 25ClF | MeOH | L | KDde | MeOH | cPrG | AA0011 |
| | Block | | | | | | | | | |
| 406 | Method | CTC | | HATU | MITS | HATU | HATU | MITS | HATU | HATU |
| | Building | Aib | | 25ClF | MeOH | L | KDde | MeOH | cPrG | AA0011 |
| | Block | | | | | | | | | |
| 407 | Method | CTC | | HATU | MITS | HATU | HATU | | HATU | HATU |
| | Building | a | | 25ClF | MeOH | L | KMe | | Nva | AA0011 |
| | Block | | | | | | | | | |
| 408 | Method | CTC | | HATU | MITS | DIC | HATU | MITS | HATU | HATU |
| | Building | Gaba | | 25ClF | Alc0046 | L | DabDde | MeOH | cPrG | AA0011 |
| | Block | | | | | | | | | |
| 409 | Method | CTC | | HATU | MITS | HATU | HATU | MITS | HATU | HATU |
| | Building | G | | 25ClF | MeOH | L | KBoc | MeOH | cPrG | AA0011 |
| | Block | | | | | | | | | |
| 410 | Method | CTC | | HATU | MITS | HATU | HATU | MITS | HATU | HATU |
| | Building | G | | 25ClF | MeOH | L | KMe | MeOH | cPrG | AA0011 |
| | Block | | | | | | | | | |
| 411 | Method | CTC | | HATU | MITS | HATU | HATU | | HATU | HATU |
| | Building | Aib | | 25ClF | MeOH | L | KBoc | | cPrG | AA0011 |
| | Block | | | | | | | | | |
| 412 | Method | CTC | | HATU | MITS | HATU | HATU | MITS | HATU | HATU |
| | Building | Aib | | 2Cl5FF | MeOH | L | KDde | MeOH | Abu | AA0011 |
| | Block | | | | | | | | | |
| 413 | Method | CTC | | HATU | MITS | HATU | HATU | MITS | HATU | HATU |
| | Building | Acpc | | 2Cl5FF | MeOH | L | KDde | MeOH | cPrG | AA0011 |
| | Block | | | | | | | | | |
| 414 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 415 | Method | CTC | | HATU | MITS | HATU | HATU | MITS | HATU | HATU |
| | Building | Aib | | 25ClF | MeOH | L | KBoc | MeOH | cPrG | Abu |
| | Block | | | | | | | | | |
| 416 | Method | CTC | | HATU | MITS | HATU | HATU | MITS | HATU | HATU |
| | Building | CFFB | | 2Cl5FF | MeOH | L | KDde | MeOH | CycBuA | AA0011 |
| | Block | | | | | | | | | |
| 417 | Method | CTC | | HATU | MITS | HATU | HATU | MITS | HATU | HATU |
| | Building | CFFB | | 25ClF | MeOH | L | KDde | MeOH | CycBuA | AA0011 |
| | Block | | | | | | | | | |
| 418 | Method | CTC | | HATU | MITS | HATU | HATU | MITS | HATU | HATU |
| | Building | CFFB | | 25FF | MeOH | L | KDde | MeOH | CycBuA | AA0011 |
| | Block | | | | | | | | | |
| 419 | Method | CTC | | HATU | MITS | HATU | HATU | MITS | HATU | HATU |
| | Building | Acpc | | 2Cl5FF | MeOH | L | KBoc | MeOH | cPrG | AA0011 |
| | Block | | | | | | | | | |
| 420 | Method | CTC | | HATU | MITS | HATU | HATU | MITS | HATU | HATU |
| | Building | p | | 2Cl5FF | MeOH | L | KBoc | MeOH | cPrG | AA0011 |
| | Block | | | | | | | | | |
| 421 | Method | CTC | | HATU | MITS | HATU | HATU | MITS | HATU | HATU |
| | Building | cFp | | 2Cl5FF | MeOH | L | KDde | MeOH | cPrG | AA0011 |
| | Block | | | | | | | | | |
| 422 | Method | CTC | | HATU | MITS | HATU | HATU | | HATU | HATU |
| | Building | Acpc | | 25ClF | MeOH | L | KBoc | | CycBuA | AA0011 |
| | Block | | | | | | | | | |
| 423 | Method | CTC | | HATU | MITS | HATU | HATU | MITS | HATU | HATU |
| | Building | a | | 25ClF | MeOH | L | KDde | MeOH | Abu | AA0011 |
| | Block | | | | | | | | | |
| 424 | Method | CTC | | HATU | MITS | HATU | HATU | MITS | HATU | HATU |
| | Building | Aib | | 25ClF | MeOH | L | KBoc | MeOH | G | AA0011 |
| | Block | | | | | | | | | |
| 425 | Method | CTC | | HATU | MITS | HATU | HATU | MITS | HATU | HATU |
| | Building | Aib | | 25ClF | MeOH | L | KDde | MeOH | cPrG | Abu |
| | Block | | | | | | | | | |
| 426 | Method | CTC | | HATU | MITS | HATU | HATU | MITS | HATU | HATU |
| | Building | cFp | | 2Cl5FF | MeOH | L | KBoc | MeOH | CycBuA | AA0011 |
| | Block | | | | | | | | | |
| 427 | Method | CTC | | HATU | MITS | HATU | HATU | MITS | HATU | HATU |
| | Building | Aib | | 25ClF | MeOH | L | KBoc | MeOH | cPrG | Abu |
| | Block | | | | | | | | | |
| 428 | Method | CTC | | HATU | MITS | HATU | HATU | MITS | HATU | HATU |
| | Building | Aib | | 25ClF | MeOH | L | KBoc | MeOH | cPrG | Abu |
| | Block | | | | | | | | | |
| 429 | Method | CTC | | HATU | MITS | HATU | HATU | MITS | HATU | HATU |
| | Building | Aib | | 25ClF | MeOH | L | KBoc | MeOH | cPrG | Abu |
| | Block | | | | | | | | | |
| 430 | Method | CTC | | HATU | MITS | DIC | HATU | MITS | HATU | HATU |
| | Building | Aib | | 25ClF | MeOH | L | KBoc | Alc0050 | cPrG | AA0011 |
| | Block | | | | | | | | | |
| 431 | Method | CTC | | HATU | MITS | HATU | HATU | | HATU | HATUnf |
| | Building | cFp | | 25ClF | MeOH | L | KBoc | | A | Acd0703 |
| | Block | | | | | | | | | |
| 432 | Method | CTC | | HATU | | HATU | HATU | | HATU | HATUnf |
| | Building | cFp | | NMe25ClF | | L | NMeK | | KDde | Acd0575 |
| | Block | | | | | | | | | |
| 433 | Method | CTC | | HATU | | HATU | HATU | | HATU | HATUnf |
| | Building | cFp | | NMe25ClF | | L | NMeK | | ODde | Acd0575 |
| | Block | | | | | | | | | |
| 434 | Method | CTC | | HATU | | HATU | HATU | | HATU | HATUnf |
| | Building | cFp | | NMe25ClF | | L | NMeK | | KDde | Acd0536 |
| | Block | | | | | | | | | |
| 435 | Method | CTC | | HATU | | HATU | HATU | | HATU | HATUnf |
| | Building | cFp | | NMe25ClF | | L | NMeK | | ODde | Acd0536 |
| | Block | | | | | | | | | |
| 436 | Method | CTC | | HATU | MITS | HATU | HATU | MITS | HATU | HATUnf |
| | Building | cFp | | 25ClF | MeOH | L | KBoc | MeOH | A | Acd0703 |
| | Block | | | | | | | | | |
| 437 | Method | CTC | | HATU | MITS | HATU | HATU | MITS | HATU | HATU |
| | Building | cFp | | 25ClF | MeOH | L | KBoc | MeOH | A | P |
| | Block | | | | | | | | | |
| 438 | Method | CTC | | HATU | | HATU | HATU | MITS | HATU | HATU |
| | Building | a | | NMe25ClF | | L | KDde | MeOH | cPrG | AA0011 |
| | Block | | | | | | | | | |
| 439 | Method | CTC | | HATU | MITS | DIC | HATU | MITS | HATU | HATU |
| | Building | Aib | | 3ClF | Alc0046 | L | KDde | MeOH | cPrG | AA0011 |
| | Block | | | | | | | | | |
| 440 | Method | CTC | | HATU | MITS | DIC | HATU | MITS | HATU | HATU |
| | Building | Acpc | | 25ClF | EtOH | L | KDde | MeOH | CycBuA | AA0011 |
| | Block | | | | | | | | | |
| 441 | Method | CTC | | HATU | MITS | DIC | HATU | MITS | HATU | HATU |
| | Building | Acpc | | 25ClF | EtOH | L | KDde | MeOH | CPA | AA0011 |
| | Block | | | | | | | | | |
| 442 | Method | CTC | | HATU | MITS | HATU | HATU | MITS | HATU | HATUnf |
| | Building | Aib | | 25ClF | MeOH | L | KBoc | MeOH | cPrG | Acd0703 |
| | Block | | | | | | | | | |
| 443 | Method | CTC | | HATU | | HATU | HATU | MITS | HATU | HATU |
| | Building | Aib | | NMe25ClF | | L | KDde | MeOH | cPrG | Tic |
| | Block | | | | | | | | | |
| 444 | Method | CTC | | HATU | | HATU | HATU | MITS | HATU | HATU |
| | Building | Aib | | NMe25ClF | | L | KDde | MeOH | cPrG | TicOH |
| | Block | | | | | | | | | |
| 445 | Method | CTC | | HATU | MITS | HATU | HATU | MITS | HATU | HATUnf |
| | Building | cFp | | 25ClF | MeOH | L | KBoc | MeOH | cPrG | Acd0733 |
| | Block | | | | | | | | | |
| 446 | Method | CTC | | HATU | MITS | HATU | HATU | MITS | HATU | HATUnf |
| | Building | Aib | | 25ClF | MeOH | L | KBoc | MeOH | cPrG | Acd0733 |
| | Block | | | | | | | | | |
| 447 | Method | CTC | | HATU | MITS | HATU | HATU | | HATU | HATUnf |
| | Building | Aib | | 25ClF | MeOH | L | KBoc | | cPrG | Acd0733 |
| | Block | | | | | | | | | |
| 448 | Method | CTC | | HATU | MITS | DIC | HATU | MITS | HATU | HATU |
| | Building | cFp | | 25ClF | Alc0046 | L | KBoc | MeOH | cPrG | AA0011 |
| | Block | | | | | | | | | |
| 449 | Method | CTC | | HATU | MITS | DIC | HATU | MITS | HATU | HATU |
| | Building | nva | | 25ClF | Alc0046 | L | KBoc | MeOH | cPrG | AA0011 |
| | Block | | | | | | | | | |
| 450 | Method | CTC | | HATU | MITS | DIC | HATU | MITS | HATU | HATU |
| | Building | nva | | 25ClF | Alc0046 | L | KMe | MeOH | cPrG | AA0011 |
| | Block | | | | | | | | | |
| 451 | Method | CTC | | HATU | MITS | DIC | HATU | MITS | HATU | HATU |
| | Building | nva | | 3ClF | EtOH | L | KBoc | MeOH | cPrG | AA0011 |
| | Block | | | | | | | | | |
| 452 | Method | CTC | | HATU | MITS | DIC | HATU | MITS | HATU | HATU |
| | Building | nva | | 3ClF | EtOH | L | KDde | MeOH | cPrG | AA0011 |
| | Block | | | | | | | | | |
| 453 | Method | CTC | | HATU | MITS | HATU | HATU | MITS | HATU | HATU |
| | Building | nva | | 25ClF | MeOH | L | KBoc | MeOH | CPA | AA0011 |
| | Block | | | | | | | | | |
| 454 | Method | CTC | | HATU | MITS | HATU | HATU | MITS | HATU | HATU |
| | Building | nva | | 25ClF | MeOH | L | KBoc | MeOH | cPrG | AA0011 |
| | Block | | | | | | | | | |
| 455 | Method | CTC | | HATU | | HATU | HATU | MITS | DIC | HATU |
| | Building | Acpc | | NMe25ClF | | L | KDde | EtOH | CycBuA | AA0011 |
| | Block | | | | | | | | | |
| 456 | Method | CTC | | HATU | MITS | DIC | HATU | MITS | HATU | HATU |
| | Building | Acpc | | 25ClF | Alc0046 | L | KDde | MeOH | CycBuA | AA0011 |
| | Block | | | | | | | | | |
| 457 | Method | CTC | | HATU | MITS | HATU | HATU | MITS | HATU | HATU |
| | Building | Acpc | | 2Cl5FF | MeOH | L | KBoc | MeOH | CycBuA | AA0011 |
| | Block | | | | | | | | | |
| 458 | Method | CTC | | HATU | MITS | HATU | HATU | MITS | HATU | HATU |
| | Building | Acpc | | 25ClF | MeOH | L | KBoc | MeOH | CycBuA | AA0011 |
| | Block | | | | | | | | | |
| 459 | Method | CTC | | HATU | MITS | HATU | HATU | MITS | HATU | HATU |
| | Building | nva | | 25ClF | MeOH | L | KBoc | MeOH | cPrG | AA0011 |
| | Block | | | | | | | | | |
| 460 | Method | CTC | | HATU | | HATU | HATU | MITS | HATU | HATU |
| | Building | nva | | NMe25ClF | | L | KBoc | MeOH | Abu | AA0011 |
| | Block | | | | | | | | | |
| 461 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 462 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 463 | Method | CTC | | HATU | MITS | HATU | HATU | MITS | HATU | HATU |
| | Building | Acpc | | 2H55ClF | MeOH | L | KDde | MeOH | cPrG | AA0011 |
| | Block | | | | | | | | | |
| 464 | Method | CTC | | HATU | MITS | HATU | HATU | MITS | HATU | HATU |
| | Building | Acpc | | 2H55ClF | MeOH | L | KBoc | MeOH | cPrG | AA0011 |
| | Block | | | | | | | | | |
| 465 | Method | CTC | | HATU | MITS | HATU | HATU | MITS | HATU | HATU |
| | Building | Acpc | | 2H55ClF | MeOH | L | KBoc | MeOH | cPrG | AA0011 |
| | Block | | | | | | | | | |
| 466 | Method | CTC | | HATU | MITS | HATU | HATU | MITS | HATU | HATUnf |
| | Building | Acpc | | 2H55ClF | MeOH | L | KBoc | MeOH | cPrG | Acd0733 |
| | Block | | | | | | | | | |
| 467 | Method | CTC | | HATU | MITS | HATU | HATU | MITS | HATU | HATU |
| | Building | Aib | | 25ClF | MeOH | L | hKBoc | MeOH | cPrG | AA0011 |
| | Block | | | | | | | | | |
| 468 | Method | CTC | | HATU | MITS | DIC | HATU | MITS | HATU | HATU |
| | Building | a | | 3ClF | EtOH | L | KDde | MeOH | A | AA0011 |
| | Block | | | | | | | | | |
| 469 | Method | CTC | | HATU | MITS | HATU | HATU | | HATU | HATU |
| | Building | a | | 25ClF | MeOH | L | KMe | | A | AA0011 |
| | Block | | | | | | | | | |
| 470 | Method | CTC | | HATU | | HATU | HATU | | HATU | HATU |
| | Building | 2aze | | NMe25ClF | | L | NMeKDde | | cPrG | AA0011 |
| | Block | | | | | | | | | |
| 471 | Method | CTC | | HATU | MITS | HATU | HATU | MITS | HATU | HATU |
| | Building | 2Aze | | 25ClF | MeOH | L | KDde | MeOH | cPrG | AA0011 |
| | Block | | | | | | | | | |
| 472 | Method | CTC | | HATU | MITS | HATU | HATU | MITS | HATU | HATU |
| | Building | Aib | | Phe0008 | MeOH | L | KBoc | MeOH | cPrG | AA0011 |
| | Block | | | | | | | | | |
| 473 | Method | CTC | | HATU | MITS | HATU | HATU | MITS | HATU | HATU |
| | Building | Acpc | | 3ClF | MeOH | L | KBoc | MeOH | CBF | AA0011 |
| | Block | | | | | | | | | |
| 474 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 475 | Method | CTC | | HATU | MITS | HATU | HATU | MITS | HATU | HATU |
| | Building | Aib | | 25ClF | MeOH | L | KDde | MeOH | cPrG | AA0011 |
| | Block | | | | | | | | | |
| 476 | Method | CTC | | HATU | MITS | HATU | HATU | MITS | HATU | HATU |
| | Building | p | | 25ClF | MeOH | L | KDde | MeOH | cPrG | AA0011 |
| | Block | | | | | | | | | |
| 477 | Method | CTC | | HATU | MITS | DIC | HATU | MITS | HATU | HATU |
| | Building | Aib | | 3ClF | EtOH | L | KDde | MeOH | cPrG | AA0011 |
| | Block | | | | | | | | | |
| 478 | Method | CTC | | HATU | MITS | HATU | HATU | MITS | HATU | HATU |
| | Building | Aib | | 20CF35ClF | MeOH | L | KBoc | MeOH | cPrG | AA0011 |
| | Block | | | | | | | | | |
| 479 | Method | CTC | | HATU | | HATU | HATU | MITS | HATU | HATU |
| | Building | Acpc | | NMe25ClF | | L | KBoc | MeOH | cPrG | AA0011 |
| | Block | | | | | | | | | |
| 480 | Method | CTC | | HATU | | HATU | HATU | | HATU | HATUnf |
| | Building | Aib | | NMe25ClF | | L | NMeK | | cPrG | Acd0741 |
| | Block | | | | | | | | | |
| 481 | Method | CTC | | HATU | MITS | HATU | HATU | MITS | HATU | HATU |
| | Building | Aib | | 25ClF | MeOH | L | KBoc | MeOH | cPrG | AA0011 |
| | Block | | | | | | | | | |
| 482 | Method | CTC | | HATU | MITS | HATU | HATU | MITS | HATU | HATU |
| | Building | Aib | | Phe0013 | MeOH | L | KDde | MeOH | cPrG | AA0011 |
| | Block | | | | | | | | | |
| 483 | Method | CTC | | HATU | MITS | HATU | HATU | MITS | HATU | HATU |
| | Building | Aib? | | 25ClF | MeOH | L | KBoc | MeOH | Mor0003 | AA0011 |
| | Block | | | | | | | | | |
| 484 | Method | CTC | | HATU | MITS | DIC | HATU | MITS | HATU | HATU |
| | Building | Aib | | 3ClF | EtOH | L | KBoc | MeOH | cPrG | AA0011 |
| | Block | | | | | | | | | |
| 485 | Method | CTC | | HATU | | HATU | HATU | MITS | HATU | HATU |
| | Building | nva | | NMe25ClF | | L | KBoc | MeOH | cPrG | AA0011 |
| | Block | | | | | | | | | |
| 486 | Method | CTC | | HATU | | HATU | HATU | | HATU | HATU |
| | Building | Aib | | NMe25ClF | | L | NMeK | | A | AA0011 |
| | Block | | | | | | | | | |
| 487 | Method | CTC | | HATU | MITS | HATU | HATU | MITS | HATU | HATU |
| | Building | Aib | | 2F5ClF | MeOH | L | KDde | MeOH | cPrG | AA0011 |
| | Block | | | | | | | | | |
| 488 | Method | CTC | | HATU | MITS | HATU | HATU | MITS | HATU | HATU |
| | Building | sHOP | | 25ClF | MeOH | L | KDde | MeOH | A | AA0011 |
| | Block | | | | | | | | | |
| 489 | Method | CTC | | HATU | MITS | HATU | HATU | MITS | HATU | HATU |
| | Building | pip | | 25ClF | MeOH | L | KBoc | MeOH | A | AA0011 |
| | Block | | | | | | | | | |
| 490 | Method | CTC | | HATU | MITS | HATU | HATU | MITS | HATU | HATU |
| | Building | 2aze | | 25ClF | MeOH | L | KDde | MeOH | A | AA0011 |
| | Block | | | | | | | | | |
| 491 | Method | CTC | | HATU | MITS | HATU | HATU | MITS | HATU | HATU |
| | Building | Aze | | 25ClF | MeOH | L | KBoc | MeOH | cPrG | AA0011 |
| | Block | | | | | | | | | |
| 492 | Method | CTC | | HATU | MITS | HATU | HATU | MITS | HATU | HATU |
| | Building | Aze | | 25ClF | MeOH | L | KBoc | MeOH | Abu | AA0011 |
| | Block | | | | | | | | | |
| 493 | Method | CTC | | HATU | MITS | HATU | HATU | MITS | HATU | HATU |
| | Building | Aib | | 25ClF | MeOH | L | KBoc | MeOH | Pip0002 | AA0011 |
| | Block | | | | | | | | | |
| 494 | Method | CTC | | HATU | MITS | HATU | HATU | MITS | HATU | HATU |
| | Building | Acpc | | 3ClF | MeOH | L | KDde | MeOH | cPrG | AA0011 |
| | Block | | | | | | | | | |
| 495 | Method | CTC | | HATU | MITS | DIC | HATU | MITS | HATU | HATU |
| | Building | Acpc | | 3ClF | EtOH | L | KDde | MeOH | cPrG | AA0011 |
| | Block | | | | | | | | | |
| 496 | Method | CTC | | HATU | MITS | DIC | HATU | MITS | HATU | HATU |
| | Building | a | | 3ClF | EtOH | L | KDde | MeOH | cPrG | AA0011 |
| | Block | | | | | | | | | |
| 497 | Method | CTC | | HATU | MITS | HATU | HATU | MITS | HATU | HATU |
| | Building | a | | 25ClF | MeOH | L | KMe | MeOH | cPrG | AA0011 |
| | Block | | | | | | | | | |
| 498 | Method | CTC | | HATU | MITS | HATU | HATU | MITS | HATU | HATUnf |
| | Building | sMe | | 25ClF | MeOH | L | KMe | MeOH | hSerMe | Acd0733 |
| | Block | | | | | | | | | |
| 499 | Method | CTC | | HATU | MITS | HATU | HATU | MITS | HATU | HATUnf |
| | Building | sMe | | 25ClF | MeOH | L | KMe | MeOH | cPrG | Acd0733 |
| | Block | | | | | | | | | |
| 500 | Method | CTC | | HATU | | HATU | HATU | MITS | DIC | HATU |
| | Building | cFp | | NMe25ClF | | L | KBoc | Alc0046 | cPrG | AA0011 |
| | Block | | | | | | | | | |
| 501 | Method | CTC | | HATU | | HATU | HATU | MITS | DIC | HATU |
| | Building | Aib | | NMe25ClF | | L | KBoc | Alc0046 | cPrG | AA0011 |
| | Block | | | | | | | | | |
| 502 | Method | CTC | | HATU | | HATU | HATU | MITS | DIC | HATU |
| | Building | Aib | | NMe25ClF | | L | KBoc | Alc0070 | cPrG | AA0011 |
| | Block | | | | | | | | | |
| 503 | Method | CTC | | HATU | MITS | HATU | HATU | | HATU | HATU |
| | Building | a | | 2H105ClF | MeOH | L | NMeK | | Abu | AA0011 |
| | Block | | | | | | | | | |
| 504 | Method | CTC | | HATU | MITS | HATU | HATU | MITS | HATU | HATU |
| | Building | a | | 2H55FF | MeOH | L | KBoc | MeOH | Abu | AA0011 |
| | Block | | | | | | | | | |
| 505 | Method | CTC | | HATU | MITS | HATU | HATU | MITS | HATU | HATU |
| | Building | Acpc | | 2H55ClF | MeOH | L | KBoc | MeOH | CycBuA | AA0011 |
| | Block | | | | | | | | | |
| 506 | Method | CTC | | HATU | MITS | HATU | HATU | MITS | DIC | HATU |
| | Building | Acpc | | 2H55ClF | MeOH | L | KBoc | Alc0070 | CycBuA | AA0011 |
| | Block | | | | | | | | | |
| 507 | Method | CTC | | HATU | MITS | HATU | HATU | MITS | DIC | HATU |
| | Building | Acpc | | 2H55ClF | MeOH | L | KBoc | Alc0070 | A | AA0011 |
| | Block | | | | | | | | | |
| 508 | Method | CTC | | HATU | MITS | HATU | HATU | MITS | DIC | HATU |
| | Building | Acpc | | 2H55ClF | MeOH | L | KBoc | Alc0070 | A | AA0011 |
| | Block | | | | | | | | | |
| 509 | Method | CTC | | HATU | MITS | HATU | HATU | | HATU | HATU |
| | Building | a | | 23Pyr5ClF | MeOH | L | NMeK | | Abu | AA0011 |
| | Block | | | | | | | | | |
| 510 | Method | CTC | | HATU | MITS | HATU | HATU | MITS | HATU | HATU |
| | Building | a | | 2H115ClF | MeOH | L | KBoc | MeOH | Abu | AA0011 |
| | Block | | | | | | | | | |
| 511 | Method | CTC | | HATU | MITS | HATU | HATU | MITS | HATU | HATU |
| | Building | sMe | | 25ClF | MeOH | L | KMe | MeOH | Abu | P |
| | Block | | | | | | | | | |
| 512 | Method | CTC | | HATU | MITS | HATU | HATU | MITS | HATU | HATU |
| | Building | sMe | | 2Me5ClF | MeOH | L | KMe | MeOH | hSerMe | P |
| | Block | | | | | | | | | |
| 513 | Method | CTC | | HATU | MITS | HATU | HATU | MITS | HATU | HATU |
| | Building | sMe | | 2H55ClF | MeOH | L | KMe | MeOH | hSerMe | P |
| | Block | | | | | | | | | |
| 514 | Method | CTC | | HATU | MITS | HATU | HATU | MITS | HATU | HATU |
| | Building | a | | 25ClF | MeOH | L | KMe | MeOH | hSerMe | P |
| | Block | | | | | | | | | |
| 515 | Method | CTC | | HATU | MITS | HATU | HATU | MITS | HATU | HATU |
| | Building | Aib | | 2Me5ClF | MeOH | L | KBoc | MeOH | cPrG | AA0011 |
| | Block | | | | | | | | | |
| 516 | Method | CTC | | HATU | MITS | HATU | HATU | MITS | HATU | HATU |
| | Building | Acpc | | 2Me5ClF | MeOH | L | KBoc | MeOH | cPrG | AA0011 |
| | Block | | | | | | | | | |
| 517 | Method | CTC | | HATU | MITS | HATU | HATU | MITS | HATU | HATU |
| | Building | nva | | 3ClF | EtOH | L | KDde | MeOH | cPrG | AA0011 |
| | Block | | | | | | | | | |
| 518 | Method | CTC | | HATU | | HATU | HATU | MITS | HATU | HATU |
| | Building | a | | NMe25ClF | | L | KMe | MeOH | CycBuA | AA0011 |
| | Block | | | | | | | | | |
| 519 | Method | CTC | | HATU | | HATU | HATU | MITS | HATU | HATU |
| | Building | Aib | | NMe25ClF | | L | KBoc | MeOH | cPrG | TicOH |
| | Block | | | | | | | | | |
| 520 | Method | CTC | | HATU | | HATU | HATU | MITS | HATU | HATU |
| | Building | Aib | | NMe25ClF | | L | KBoc | MeOH | cPrG | TicOH |
| | Block | | | | | | | | | |
| 521 | Method | CTC | | HATU | | HATU | HATU | MITS | HATU | HATU |
| | Building | Aib | | NMe25ClF | | L | KBoc | MeOH | cPrG | TicOH |
| | Block | | | | | | | | | |
| 522 | Method | CTC | | HATU | | HATU | HATU | MITS | HATU | HATU |
| | Building | Aib | | NMe25ClF | | L | KBoc | MeOH | cPrG | Tic |
| | Block | | | | | | | | | |
| 523 | Method | CTC | | HATU | | HATU | HATU | MITS | HATU | HATUnf |
| | Building | abu | | NMe25ClF | | L | KMe | MeOH | A | Acd0703 |
| | Block | | | | | | | | | |
| 524 | Method | CTC | | HATU | MITS | HATU | HATU | MITS | DIC | HATU |
| | Building | Aib | | 2Me5ClF | MeOH | L | KBoc | EtOH | cPrG | AA0011 |
| | Block | | | | | | | | | |
| 525 | Method | CTC | | HATU | MITS | HATU | HATU | MITS | DIC | HATU |
| | Building | Aib | | 2Me5ClF | MeOH | L | KBoc | EtOH | cPrG | AA0011 |
| | Block | | | | | | | | | |
| 526 | Method | CTC | | HATU | MITS | HATU | HATU | MITS | DIC | HATU |
| | Building | 2aze | | 2Me5ClF | MeOH | L | KBoc | EtOH | cPrG | AA0011 |
| | Block | | | | | | | | | |
| 527 | Method | CTC | | HATU | MITS | HATU | HATU | MITS | DIC | HATU |
| | Building | 2aze | | 2Me5ClF | MeOH | L | KDde | EtOH | cPrG | AA0011 |
| | Block | | | | | | | | | |
| 528 | Method | CTC | | HATU | MITS | HATU | HATU | MITS | DIC | HATUnf |
| | Building | abu | | 2Me5ClF | MeOH | L | KMe | EtOH | A | Acd0536 |
| | Block | | | | | | | | | |
| 529 | Method | CTC | | HATU | MITS | HATU | HATU | MITS | DIC | HATUnf |
| | Building | abu | | 2H55ClF | MeOH | L | KMe | EtOH | A | Acd0536 |
| | Block | | | | | | | | | |
| 530 | Method | CTC | | HATU | | HATU | HATU | MITS | DIC | HATU |
| | Building | abu | | NMe25ClF | | L | KMe | EtOH | cPrG | P |
| | Block | | | | | | | | | |
| 531 | Method | CTC | | HATU | | HATU | HATU | MITS | DIC | HATU |
| | Building | a | | NMe25ClF | | L | KMe | Alc0046 | A | P |
| | Block | | | | | | | | | |
| 532 | Method | CTC | | HATU | MITS | HATU | HATU | MITS | DIC | HATU |
| | Building | a | | 2H55ClF | MeOH | L | KMe | EtOH | cPrG | AA0011 |
| | Block | | | | | | | | | |
| 533 | Method | CTC | | HATU | MITS | HATU | HATU | MITS | HATU | HATU |
| | Building | a | | 2H55ClF | MeOH | L | KMe | MeOH | cPrG | AA0011 |
| | Block | | | | | | | | | |
| 534 | Method | CTC | | HATU | MITS | HATU | HATU | MITS | HATU | HATU |
| | Building | a | | 20CF35ClF | MeOH | L | KMe | MeOH | cPrG | AA0011 |
| | Block | | | | | | | | | |
| 535 | Method | CTC | | HATU | MITS | HATU | HATU | MITS | DIC | HATU |
| | Building | a | | 2H55ClF | MeOH | L | KMe | EtOH | cPrG | AA0011 |
| | Block | | | | | | | | | |
| 536 | Method | CTC | | HATU | MITS | HATU | HATU | MITS | DIC | HATU |
| | Building | a | | 2H55ClF | MeOH | L | KMe | EtOH | CycBuA | AA0011 |
| | Block | | | | | | | | | |
| 537 | Method | CTC | | HATU | MITS | HATU | HATU | MITS | DIC | HATU |
| | Building | a | | 2H55ClF | MeOH | L | KMe | MeOH | cPrG | AA0011 |
| | Block | | | | | | | | | |
| 538 | Method | CTC | | HATU | MITS | HATU | HATU | MITS | DIC | HATU |
| | Building | a | | 2H55ClF | MeOH | L | KMe | MeOH | CycBuA | AA0011 |
| | Block | | | | | | | | | |
| 539 | Method | CTC | | HATU | MITS | HATU | HATU | MITS | DIC | HATU |
| | Building | a | | 2Me5ClF | MeOH | L | KMe | EtOH | cPrG | AA0011 |
| | Block | | | | | | | | | |
| 540 | Method | CTC | | HATU | MITS | HATU | HATU | MITS | HATU | HATU |
| | Building | a | | 20CF35ClF | MeOH | L | KMe | MeOH | cPrG | AA0011 |
| | Block | | | | | | | | | |
| 541 | Method | CTC | | HATU | MITS | HATU | HATU | MITS | DIC | HATU |
| | Building | a | | 2Me5ClF | MeOH | L | KDde | EtOH | cPrG | AA0011 |
| | Block | | | | | | | | | |
| 542 | Method | CTC | | HATU | MITS | HATU | HATU | MITS | DIC | HATU |
| | Building | a | | 2Me5ClF | MeOH | L | KDde | EtOH | CycBuA | AA0011 |
| | Block | | | | | | | | | |
| 543 | Method | CTC | | HATU | MITS | HATU | HATU | MITS | DIC | HATU |
| | Building | a | | 2H55ClF | MeOH | L | KDde | EtOH | cPrG | AA0011 |
| | Block | | | | | | | | | |
| 544 | Method | CTC | | HATU | MITS | HATU | HATU | MITS | DIC | HATU |
| | Building | a | | 2H55ClF | MeOH | L | KDde | EtOH | CycBuA | AA0011 |
| | Block | | | | | | | | | |
| 545 | Method | CTC | | HATU | | HATU | HATU | MITS | DIC | HATU |
| | Building | a | | NMe25ClF | | L | KMe | EtOH | cPrG | AA0011 |
| | Block | | | | | | | | | |
| 546 | Method | CTC | | HATU | | HATU | HATU | MITS | HATU | HATU |
| | Building | Aib | | NMe25ClF | | L | KBoc | MeOH | cPrG | Tic0004 |
| | Block | | | | | | | | | |
| 547 | Method | CTC | | HATU | | HATU | HATU | MITS | HATU | HATU |
| | Building | Aib | | NMe25ClF | | L | KDde | MeOH | cPrG | Tic0005 |
| | Block | | | | | | | | | |
| 548 | Method | CTC | | HATU | | HATU | HATU | MITS | HATU | HATU |
| | Building | Aib | | NMe25ClF | | L | KBoc | MeOH | cPrG | Tic0005 |
| | Block | | | | | | | | | |
| 549 | Method | CTC | | HATU | MITS | DIC | HATU | | HATU | HATU |
| | Building | nva | | 5Cl2OcPrF | EtOH | L | NMeKDde | | cPrG | AA0011 |
| | Block | | | | | | | | | |
| 550 | Method | CTC | | HATU | | HATU | HATU | MITS | HATU | HATU |
| | Building | nva | | NMe25ClF | | L | KBoc | MeOH | cPrG | AA0011 |
| | Block | | | | | | | | | |
| 551 | Method | CTC | | HATU | | HATU | HATU | MITS | HATU | HATU |
| | Building | nva | | NMe25ClF | | L | KMe | MeOH | cPrG | AA0011 |
| | Block | | | | | | | | | |
| 552 | Method | CTC | | HATU | | HATU | HATU | | HATU | HATU |
| | Building | Aib | | NMe25ClF | | L | NMeK | | cPrG | SHOP |
| | Block | | | | | | | | | |
| 553 | Method | CTC | | HATU | | HATU | HATU | MITS | HATU | HATU |
| | Building | Aib | | NMe25ClF | | L | KDde | MeOH | cPrG | SHOP |
| | Block | | | | | | | | | |
| 554 | Method | CTC | | HATU | MITS | HATU | HATU | MITS | HATU | HATUnf |
| | Building | abu | | 2Cl5FF | MeOH | L | KMe | MeOH | A | Acd0536 |
| | Block | | | | | | | | | |
| 555 | Method | CTC | | HATU | MITS | HATU | HATU | MITS | HATU | HATUnf |
| | Building | abu | | 3ClF | MeOH | L | KMe | MeOH | A | Acd0536 |
| | Block | | | | | | | | | |
| 556 | Method | CTC | | HATU | MITS | HATU | HATU | MITS | HATU | HATUnf |
| | Building | abu | | 2Cl5FF | MeOH | L | KMe | EtOH | A | Acd0536 |
| | Block | | | | | | | | | |
| 557 | Method | CTC | | HATU | MITS | HATU | HATU | MITS | HATU | HATU |
| | Building | nva | | 2Cl5FF | MeOH | L | KDde | MeOH | cPrG | AA0011 |
| | Block | | | | | | | | | |
| 558 | Method | CTC | | HATU | MITS | HATU | HATU | MITS | HATU | HATU |
| | Building | nva | | 3ClF | MeOH | L | KDde | MeOH | cPrG | AA0011 |
| | Block | | | | | | | | | |
| 559 | Method | CTC | | HATU | | HATU | HATU | | HATU | HATU |
| | Building | Aib | | NMe25ClF | | L | NMeK | | cPrG | NMeA |
| | Block | | | | | | | | | |
| 560 | Method | CTC | | HATU | MITS | HATU | HATU | MITS | DIC | HATUnf |
| | Building | G | | 25ClF | MeOH | L | KMe | EtOH | A | Acd0536 |
| | Block | | | | | | | | | |
| 561 | Method | CTC | | HATU | MITS | HATU | HATU | MITS | DIC | HATU |
| | Building | abu | | 25ClF | MeOH | L | KMe | EtOH | A | P |
| | Block | | | | | | | | | |
| 562 | Method | CTC | | HATU | | HATU | HATU | | HATU | HATU |
| | Building | G | | NMe25ClF | | L | KBoc | | Abu | AA0011 |
| | Block | | | | | | | | | |
| 563 | Method | CTC | | HATU | | HATU | HATU | MITS | HATU | HATU |
| | Building | Acpc | | NMe25ClF | | L | KMe | MeOH | CycBuA | AA0011 |
| | Block | | | | | | | | | |
| 564 | Method | CTC | | HATU | MITS | HATU | HATU | MITS | HATU | HATU |
| | Building | a | | 25ClF | MeOH | L | KMe | MeOH | cPrG | P |
| | Block | | | | | | | | | |
| 565 | Method | CTC | | HATU | | HATU | HATU | | HATU | HATU |
| | Building | a | | NMe25ClF | | L | NMeK | | cPrG | P |
| | Block | | | | | | | | | |
| 566 | Method | CTC | | HATU | MITS | HATU | HATU | MITS | DIC | HATU |
| | Building | nva | | 25ClF | MeOH | L | KBoc | Alc0070 | cPrG | AA0011 |
| | Block | | | | | | | | | |
| 567 | Method | CTC | | HATU | | HATU | HATU | MITS | DIC | HATU |
| | Building | nva | | NMe25ClF | | L | KMe | Alc0070 | cPrG | AA0011 |
| | Block | | | | | | | | | |
| 568 | Method | CTC | | HATU | MITS | HATU | HATU | MITS | HATU | HATU |
| | Building | nva | | 2H55ClF | MeOH | L | KMe | MeOH | cPrG | AA0011 |
| | Block | | | | | | | | | |
| 569 | Method | CTC | | HATU | MITS | HATU | HATU | MITS | HATU | HATU |
| | Building | nva | | 25ClF | MeOH | L | KMe | MeOH | cPrG | AA0011 |
| | Block | | | | | | | | | |
| 570 | Method | CTC | | HATU | MITS | HATU | HATU | MITS | HATU | HATU |
| | Building | abu | | 25ClF | MeOH | L | KMe | MeOH | cPrG | AA0011 |
| | Block | | | | | | | | | |
| 571 | Method | CTC | | HATU | MITS | HATU | HATU | MITS | HATU | HATU |
| | Building | abu | | NMe25ClF | | L | KMe | MeOH | cPrG | TicOH |
| | Block | | | | | | | | | |
| 572 | Method | CTC | | HATU | MITS | HATU | HATU | MITS | HATU | HATU |
| | Building | abu | | NMe25ClF | | L | NMeKMe | | cPrG | AA0011 |
| | Block | | | | | | | | | |
| 573 | Method | CTC | | HATU | MITS | HATU | HATU | MITS | DIC | HATU |
| | Building | a | | 25ClF | MeOH | L | KDde | Alc0070 | cPrG | AA0011 |
| | Block | | | | | | | | | |
| 574 | Method | CTC | | HATU | MITS | HATU | HATU | MITS | HATU | HATU |
| | Building | abu | | 25ClF | MeOH | L | KMe | MeOH | CycBuA | AA0011 |
| | Block | | | | | | | | | |
| 575 | Method | CTC | | HATU | MITS | HATU | HATU | MITS | HATU | HATU |
| | Building | abu | | 25ClF | MeOH | L | KMe | MeOH | Abu | AA0011 |
| | Block | | | | | | | | | |
| 576 | Method | CTC | | HATU | | HATU | HATU | MITS | DIC | HATU |
| | Building | abu | | NMe25ClF | | L | KMe | Alc0070 | cPrG | AA0011 |
| | Block | | | | | | | | | |
| 577 | Method | CTC | | HATU | MITS | HATU | HATU | | HATU | HATU |
| | Building | abu | | Phe0013 | MeOH | L | NMeKDde | | cPrG | AA0011 |
| | Block | | | | | | | | | |
| 578 | Method | CTC | | HATU | | HATU | HATU | | HATU | HATUnf |
| | Building | abu | | NMe25ClF | | L | NMeKMe | | cPrG | Acd0703 |
| | Block | | | | | | | | | |
| 579 | Method | CTC | | HATU | | HATU | HATU | MITS | DIC | HATU |
| | Building | p | | NMe25ClF | | I | KDde | EtOH | cPrG | AA0011 |
| | Block | | | | | | | | | |
| 580 | Method | CTC | | HATU | MITS | HATU | HATU | MITS | DIC | HATU |
| | Building | a | | 25ClF | MeOH | L | KMe | Alc0070 | A | AA0011 |
| | Block | | | | | | | | | |
| 581 | Method | CTC | | HATU | | HATU | HATU | MITS | DIC | HATU |
| | Building | a | | NMe25ClF | | L | KMe | Alc0070 | CPA | AA0011 |
| | Block | | | | | | | | | |
| 582 | Method | CTC | | HATU | MITS | HATU | HATU | MITS | HATU | HATU |
| | Building | a | | 2H55ClF | MeOH | L | KMe | MeOH | cPrG | TicOH |
| | Block | | | | | | | | | |
| 583 | Method | CTC | | HATU | MITS | HATU | HATU | MITS | HATU | HATU |
| | Building | sMe | | 2H55ClF | MeOH | L | KMe | MeOH | hSerMe | AA0011 |
| | Block | | | | | | | | | |
| 584 | Method | CTC | | HATU | | HATU | HATU | MITS | DIC | HATU |
| | Building | Aib | | NMe25ClF | | L | KBoc | Alc0070 | cPrG | AA0011 |
| | Block | | | | | | | | | |
| 585 | Method | CTC | | HATU | MITS | HATU | HATU | MITS | DIC | HATU |
| | Building | Aib | | 2H55ClF | MeOH | L | KBoc | Alc0070 | cPrG | AA0011 |
| | Block | | | | | | | | | |
| 586 | Method | CTC | | HATU | MITS | HATU | HATU | MITS | DIC | HATU |
| | Building | Aib | | 2H55ClF | MeOH | L | KBoc | Alc0070 | cPrG | AA0011 |
| | Block | | | | | | | | | |
| 587 | Method | CTC | | HATU | MITS | HATU | HATU | MITS | HATU | HATU |
| | Building | Acpc | | 25ClF | MeOH | L | KBoc | MeOH | CPA | AA0011 |
| | Block | | | | | | | | | |
| 588 | Method | CTC | | HATU | | HATU | HATU | MITS | HATU | HATU |
| | Building | a | | NMe25ClF | | L | KMe | MeOH | CPA | AA0011 |
| | Block | | | | | | | | | |
| 589 | Method | CTC | | HATU | MITS | HATU | HATU | MITS | HATU | HATU |
| | Building | a | | 2H55ClF | MeOH | L | KMe | MeOH | Mor0003 | JAA0011 |
| | Block | | | | | | | | | |
| 590 | Method | CTC | | HATU | MITS | HATU | HATU | | HATU | HATU |
| | Building | Aib | | 2H55FF | MeOH | L | NMeK | | cPrG | AA0011 |
| | Block | | | | | | | | | |
| 591 | Method | CTC | | HATU | MITS | HATU | HATU | | HATU | HATU |
| | Building | Aib | | 2H115ClF | MeOH | L | NMeK | | cPrG | AA0011 |
| | Block | | | | | | | | | |
| 592 | Method | CTC | | HATU | MITS | HATU | HATU | | HATU | HATU |
| | Building | Aib | | 2H135ClF | MeOH | L | NMeK | | cPrG | AA0011 |
| | Block | | | | | | | | | |
| 593 | Method | CTC | | HATU | MITS | HATU | HATU | | HATU | HATU |
| | Building | Aib | | 2H125ClF | MeOH | L | NMeK | | cPrG | AA0011 |
| | Block | | | | | | | | | |
| 594 | Method | CTC | | HATU | MITS | HATU | HATU | | HATU | HATU |
| | Building | Aib | | 2H165ClF | MeOH | L | NMeK | | cPrG | AA0011 |
| | Block | | | | | | | | | |
| 595 | Method | CTC | | HATU | MITS | HATU | HATU | | HATU | HATU |
| | Building | Aib | | 2H145ClF | MeOH | L | NMeK | | cPrG | AA0011 |
| | Block | | | | | | | | | |
| 596 | Method | CTC | | HATU | MITS | HATU | HATU | | HATU | HATU |
| | Building | Aib | | 2H105ClF | MeOH | L | NMeK | | cPrG | AA0011 |
| | Block | | | | | | | | | |
| 597 | Method | CTC | | HATU | MITS | HATU | HATU | | HATU | HATU |
| | Building | Aib | | 2H95ClF | MeOH | L | NMeK | | cPrG | AA0011 |
| | Block | | | | | | | | | |
| 598 | Method | CTC | | HATU | MITS | HATU | HATU | | HATU | HATU |
| | Building | Aib | | 2H155ClF | MeOH | L | NMeK | | cPrG | AA0011 |
| | Block | | | | | | | | | |
| 599 | Method | CTC | | HATU | | HATU | HATU | MITS | DIC | HATUnf |
| | Building | SHOP | | NMe25ClF | | L | KDde | Alc0046 | A | Acd0536 |
| | Block | | | | | | | | | |
| 600 | Method | CTC | | HATU | | HATU | HATU | | HATU | HATUnf |
| | Building | sHOp | | NMe25ClF | | L | NMeKDde | | A | Acd0536 |
| | Block | | | | | | | | | |
| 601 | Method | CTC | | HATU | | HATU | HATU | MITS | DIC | HATUnf |
| | Building | sHOP | | NMe25ClF | | L | KDde | EtOH | A | Acd0536 |
| | Block | | | | | | | | | |
| 602 | Method | CTC | | HATU | | HATU | HATU | MITS | DIC | HATU |
| | Building | p | | NMe25ClF | | L | KBoc | Alc0046 | A | AA0011 |
| | Block | | | | | | | | | |
| 603 | Method | CTC | | HATU | | HATU | HATU | MITS | DIC | HATU |
| | Building | sHOP | | NMe25ClF | | L | KDde | Alc0046 | A | AA0011 |
| | Block | | | | | | | | | |
| 604 | Method | CTC | | HATU | | HATU | HATU | MITS | DIC | HATU |
| | Building | Aib | | NMe25ClF | | L | KDde | Alc0070 | cPrG | AA0011 |
| | Block | | | | | | | | | |
| 605 | Method | CTC | | HATU | MITS | HATU | HATU | MITS | DIC | HATU |
| | Building | Aib | | 2H55ClF | MeOH | L | KDde | Alc0070 | cPrG | AA0011 |
| | Block | | | | | | | | | |
| 606 | Method | CTC | | HATU | | HATU | HATU | MITS | DIC | HATU |
| | Building | Aib | | NMe25ClF | | L | KDde | Alc0046 | cPrG | AA0011 |
| | Block | | | | | | | | | |
| 607 | Method | CTC | | HATU | MITS | HATU | HATU | MITS | DIC | HATU |
| | Building | Aib | | 2H55ClF | MeOH | L | KDde | Alc0046 | cPrG | AA0011 |
| | Block | | | | | | | | | |
| 608 | Method | CTC | | HATU | MITS | HATU | HATU | MITS | DIC | HATU |
| | Building | SHOP | | 2H55ClF | MeOH | L | KDde | Alc0046 | A | AA0011 |
| | Block | | | | | | | | | |
| 609 | Method | CTC | | HATU | MITS | HATU | HATU | MITS | DIC | HATU |
| | Building | sHOP | | 2H55ClF | MeOH | L | KDde | Alc0046 | A | AA0011 |
| | Block | | | | | | | | | |
| 610 | Method | CTC | | HATU | | HATU | HATU | MITS | DIC | HATU |
| | Building | Aib | | NMe25ClF | | L | KDde | Alc0046 | cPrG | TicOH |
| | Block | | | | | | | | | |
| 611 | Method | CTC | | HATU | MITS | HATU | HATU | MITS | DIC | HATU |
| | Building | Aib | | 2H55ClF | MeOH | L | KBoc | Alc0046 | cPrG | TicOH |
| | Block | | | | | | | | | |
| 612 | Method | CTC | | HATU | MITS | HATU | HATU | | HATU | HATU |
| | Building | SHOP | | 2H55ClF | MeOH | L | NMeKDde | | A | AA0011 |
| | Block | | | | | | | | | |
| 613 | Method | CTC | | HATU | | HATU | HATU | MITS | DIC | HATU |
| | Building | SHOP | | NMe25ClF | | L | KDde | Alc0046 | A | AA0011 |
| | Block | | | | | | | | | |
| 614 | Method | CTC | | HATU | MITS | HATU | HATU | | HATU | HATU |
| | Building | SHOP | | 2H55ClF | MeOH | L | NMeKDde | | A | AA0011 |
| | Block | | | | | | | | | |
| 615 | Method | CTC | | HATU | MITS | HATU | HATU | MITS | DIC | HATU |
| | Building | abu | | 25ClF | MeOH | L | KBoc | Alc0050 | KDde | AA0011 |
| | Block | | | | | | | | | |
| 616 | Method | CTC | | HATU | | HATU | HATU | | HATU | HATU |
| | Building | abu | | NMe2H55ClF | | L | NMeKDde | | KMor | AA0011 |
| | Block | | | | | | | | | |
| 617 | Method | CTC | | HATU | | HATU | HATU | MITS | DIC | HATU |
| | Building | Aib | | NMe25ClF | | L | KBoc | Alc0046 | A | AA0011 |
| | Block | | | | | | | | | |
| 618 | Method | CTC | | HATU | MITS | HATU | HATU | | HATU | HATU |
| | Building | Aib | | 2H55ClF | MeOH | L | NMeK | | A | AA0011 |
| | Block | | | | | | | | | |
| 619 | Method | CTC | | HATU | MITS | HATU | HATU | | HATU | HATU |
| | Building | Acpc | | 2H55ClF | MeOH | L | NMeK | | cPrG | AA0011 |
| | Block | | | | | | | | | |
| 620 | Method | CTC | | HATU | MITS | HATU | HATU | MITS | HATU | HATU |
| | Building | Acpc | | 2H55ClF | MeOH | L | KMe | MeOH | cPrG | AA0011 |
| | Block | | | | | | | | | |
| 621 | Method | CTC | | HATU | MITS | HATU | HATU | | HATU | HATU |
| | Building | Acpc | | 2H55ClF | MeOH | L | NMeK | | cPrG | P |
| | Block | | | | | | | | | |
| 622 | Method | CTC | | HATU | | HATU | HATU | | HATU | HATUnf |
| | Building | abu | | NMe25ClF | | L | NMeK | | A | Acd0801 |
| | Block | | | | | | | | | |
| 623 | Method | CTC | | HATU | MITS | HATU | HATU | | HATU | HATU |
| | Building | Aib | | 2Ph5ClF | MeOH | L | NMeKDde | | cPrG | AA0011 |
| | Block | | | | | | | | | |
| 624 | Method | CTC | | HATU | MITS | HATU | HATU | | HATU | HATU |
| | Building | Aib | | 23Pyr5ClF | MeOH | L | NMeK | | cPrG | AA0011 |
| | Block | | | | | | | | | |
| 625 | Method | CTC | | HATU | | HATU | HATU | | HATU | HATUnf |
| | Building | abu | | NMe25ClF | | L | NMeKDde | | A | Acd0794 |
| | Block | | | | | | | | | |
| 626 | Method | CTC | | HATU | MITS | HATU | HATU | | HATU | HATU |
| | Building | Aib | | 5Cl2OcHexF | MeOH | L | NMeK | | cPrG | AA0011 |
| | Block | | | | | | | | | |
| 627 | Method | CTC | | HATU | MITS | HATU | HATU | | HATU | HATU |
| | Building | Aib | | 5Cl2OcPrF | MeOH | L | NMeK | | cPrG | AA0011 |
| | Block | | | | | | | | | |
| 628 | Method | CTC | | HATU | MITS | HATU | HATU | | HATU | HATU |
| | Building | Aib | | Phe0034 | MeOH | L | NMeK | | cPrG | AA0011 |
| | Block | | | | | | | | | |
| 629 | Method | CTC | | HATU | MITS | HATU | HATU | | HATU | HATU |
| | Building | Aib | | 5Cl2OPhF | MeOH | L | NMeK | | cPrG | AA0011 |
| | Block | | | | | | | | | |
| 630 | Method | CTC | | HATU | MITS | HATU | HATU | | HATU | HATU |
| | Building | Aib | | 5Cl2OcHexF | MeOH | L | NMeKDde | | cPrG | AA0011 |
| | Block | | | | | | | | | |
| 631 | Method | CTC | | HATU | MITS | HATU | HATU | | HATU | HATU |
| | Building | Aib | | Phe0034 | MeOH | L | NMeKDde | | cPrG | AA0011 |
| | Block | | | | | | | | | |
| 632 | Method | CTC | | HATU | MITS | HATU | HATU | | HATU | HATU |
| | Building | Aib | | 5Cl2OPhF | MeOH | L | NMeKDde | | cPrG | AA0011 |
| | Block | | | | | | | | | |
| 633 | Method | CTC | | HATU | | HATU | HATU | MITS | DIC | HATU |
| | Building | nva | | NMe25ClF | | L | KBoc | Alc0070 | cPrG | P |
| | Block | | | | | | | | | |
| 634 | Method | CTC | | HATU | MITS | HATU | HATU | MITS | HATU | HATU |
| | Building | a | | 2H55ClF | MeOH | L | KMe | MeOH | CycBuA | P |
| | Block | | | | | | | | | |
| 635 | Method | CTC | | HATU | | HATU | HATU | | HATU | HATU |
| | Building | Aib | | NMe25ClF | | L | NMeKDde | | cPrG | AA0011 |
| | Block | | | | | | | | | |
| 636 | Method | CTC | | HATU | | HATU | HATU | | HATU | HATU |
| | Building | Aib | | NMe25ClF | | L | NMeKDde | | cPrG | AA0011 |
| | Block | | | | | | | | | |
| 637 | Method | CTC | | HATU | MITS | HATU | HATU | | HATU | HATU |
| | Building | a | | 2H55ClF | MeOH | L | NMeKDde | | cPrG | AA0011 |
| | Block | | | | | | | | | |
| 638 | Method | CTC | | HATU | | HATU | HATU | | HATU | HATUnf |
| | Building | a | | NMe2H55ClF | | L | NMeKMe | | cPrG | Acd0799 |
| | Block | | | | | | | | | |
| 639 | Method | CTC | | HATU | | HATU | HATU | MITS | DIC | HATU |
| | Building | cFp | | NMe25ClF | | L | KBoc | Alc0070 | cPrG | AA0011 |
| | Block | | | | | | | | | |
| 640 | Method | CTC | | HATU | | HATU | HATU | MITS | DIC | HATU |
| | Building | cFp | | NMe25ClF | | L | KBoc | Alc0070 | cPrG | AA0011 |
| | Block | | | | | | | | | |
| 641 | Method | CTC | | HATU | MITS | HATU | HATU | MITS | HATU | HATU |
| | Building | a | | 2H55ClF | MeOH | L | KMe | MeOH | cPrG | AA0011 |
| | Block | | | | | | | | | |
| 642 | Method | CTC | | HATU | | HATU | HATU | | HATU | HATU |
| | Building | Aib | | NMe25ClF | | L | NMeK | | cPrG | AA0011 |
| | Block | | | | | | | | | |
| 643 | Method | CTC | | HATU | MITS | HATU | HATU | MITS | HATU | HATU |
| | Building | a | | 2H55ClF | MeOH | L | KMe | MeOH | cPrG | AA0011 |
| | Block | | | | | | | | | |
| 644 | Method | CTC | | HATU | | HATU | HATU | | HATU | HATU |
| | Building | Aib | | NMe25ClF | | L | NMeK | | cPrG | AA0011 |
| | Block | | | | | | | | | |
| 645 | Method | CTC | | HATU | MITS | HATU | HATU | MITS | HATU | HATU |
| | Building | a | | 2H55ClF | MeOH | L | KMe | MeOH | cPrG | AA0011 |
| | Block | | | | | | | | | |
| 646 | Method | CTC | | HATU | | HATU | HATU | | HATU | HATU |
| | Building | Aib | | NMe25ClF | | L | NMeK | | cPrG | AA0011 |
| | Block | | | | | | | | | |
| 647 | Method | CTC | | HATU | MITS | HATU | HATU | | HATU | HATU |
| | Building | a | | 2H55ClF | MeOH | L | NMeKDde | | cPrG | AA0011 |
| | Block | | | | | | | | | |
| 648 | Method | CTC | | HATU | | HATU | HATU | | HATU | HATU |
| | Building | cFp | | NMe25ClF | | L | NMeK | | CycBuA | AA0011 |
| | Block | | | | | | | | | |
| 649 | Method | CTC | | HATU | MITS | DIC | HATU | | HATU | HATU |
| | Building | cFp | | 25ClF | Alc0046 | L | NMeKDde | | CycBuA | AA0011 |
| | Block | | | | | | | | | |
| 650 | Method | CTC | | HATU | | HATU | HATU | | HATU | HATU |
| | Building | Acpc | | NMe25ClF | | L | NMeK | | A | AA0011 |
| | Block | | | | | | | | | |
| 651 | Method | CTC | | HATU | MITS | HATU | HATU | | HATU | HATU |
| | Building | cFp | | 2H55ClF | MeOH | L | NMeK | | A | AA0011 |
| | Block | | | | | | | | | |
| 652 | Method | CTC | | HATU | MITS | HATU | HATU | | HATU | HATU |
| | Building | Aib | | Phe0023 | MeOH | L | NMeK | | cPrG | AA0011 |
| | Block | | | | | | | | | |
| 653 | Method | CTC | | HATU | MITS | HATU | HATU | | HATU | HATU |
| | Building | Aib | | Phe0024 | MeOH | L | NMeK | | cPrG | AA0011 |
| | Block | | | | | | | | | |
| 654 | Method | CTC | | HATU | MITS | HATU | HATU | | HATU | HATU |
| | Building | Aib | | 5Cl2OMePen | MeOH | L | NMeK | | cPrG | AA0011 |
| | Block | | | | | | | | | |
| 655 | Method | CTC | | HATU | MITS | HATU | HATU | | HATU | HATU |
| | Building | Aib | | 5Cl2OcPenF | MeOH | L | NMeK | | cPrG | AA0011 |
| | Block | | | | | | | | | |
| 656 | Method | CTC | | HATU | MITS | HATU | HATU | | HATU | HATU |
| | Building | Aib | | 20CF35ClF | MeOH | L | NMeK | | cPrG | AA0011 |
| | Block | | | | | | | | | |
| 657 | Method | CTC | | HATU | MITS | HATU | HATU | | HATU | HATU |
| | Building | Aib | | Phe0013 | MeOH | L | NMeK | | cPrG | AA0011 |
| | Block | | | | | | | | | |
| 658 | Method | CTC | | HATU | MITS | HATU | HATU | | HATU | HATU |
| | Building | Aib | | 20EtCF35ClF | MeOH | L | NMeK | | cPrG | AA0011 |
| | Block | | | | | | | | | |
| 659 | Method | CTC | | HATU | MITS | HATU | HATU | | HATU | HATU |
| | Building | Aib | | 2Cl5FF | MeOH | L | NMeK | | cPrG | AA0011 |
| | Block | | | | | | | | | |
| 660 | Method | CTC | | HATU | | HATU | HATU | | HATU | HATU |
| | Building | Aib | | NMe25ClF | | L | NMeK | | CycBuA | AA0011 |
| | Block | | | | | | | | | |
| 661 | Method | CTC | | HATU | | HATU | HATU | | HATU | HATU |
| | Building | Aib | | NMe25ClF | | L | NMeK | | hSerMe | AA0011 |
| | Block | | | | | | | | | |
| 662 | Method | CTC | | HATU | MITS | HATU | HATU | | HATU | HATU |
| | Building | Aib | | Phe0062 | MeOH | L | NMeK | | cPrG | AA0011 |
| | Block | | | | | | | | | |
| 663 | Method | CTC | | HATU | MITS | HATU | HATU | | HATU | HATU |
| | Building | Aib | | Phe0042 | MeOH | L | NMeK | | cPrG | AA0011 |
| | Block | | | | | | | | | |
| 664 | Method | CTC | | HATU | MITS | HATU | HATU | | HATU | HATU |
| | Building | Aib | | Phe0046 | MeOH | L | NMeK | | cPrG | AA0011 |
| | Block | | | | | | | | | |
| 665 | Method | CTC | | HATU | MITS | HATU | HATU | | HATU | HATU |
| | Building | Aib | | Phe0047 | MeOH | L | NMeK | | cPrG | AA0011 |
| | Block | | | | | | | | | |
| 666 | Method | CTC | | HATU | MITS | HATU | HATU | | HATU | HATU |
| | Building | Aib | | Phe0048 | MeOH | L | NMeK | | cPrG | AA0011 |
| | Block | | | | | | | | | |
| 667 | Method | CTC | | HATU | MITS | HATU | HATU | | HATU | HATU |
| | Building | Aib | | Phe0049 | MeOH | L | NMeK | | cPrG | AA0011 |
| | Block | | | | | | | | | |
| 668 | Method | CTC | | HATU | MITS | HATU | HATU | | HATU | HATU |
| | Building | Aib | | Phe0050 | MeOH | L | NMeK | | cPrG | AA0011 |
| | Block | | | | | | | | | |
| 669 | Method | CTC | | HATU | MITS | HATU | HATU | | HATU | HATU |
| | Building | Aib | | Phe0051 | MeOH | L | NMeK | | cPrG | AA0011 |
| | Block | | | | | | | | | |
| 670 | Method | CTC | | HATU | MITS | HATU | HATU | | HATU | HATU |
| | Building | Aib | | Phe0053 | MeOH | L | NMeK | | cPrG | AA0011 |
| | Block | | | | | | | | | |
| 671 | Method | CTC | | HATU | MITS | HATU | HATU | | HATU | HATU |
| | Building | Aib | | Phe0055 | MeOH | L | NMeK | | cPrG | AA0011 |
| | Block | | | | | | | | | |
| 672 | Method | CTC | | HATU | MITS | HATU | HATU | | HATU | HATU |
| | Building | Aib | | Phe0056 | MeOH | L | NMeK | | cPrG | AA0011 |
| | Block | | | | | | | | | |
| 673 | Method | CTC | | HATU | MITS | HATU | HATU | | HATU | HATU |
| | Building | Aib | | Phe0057 | MeOH | L | NMeK | | cPrG | AA0011 |
| | Block | | | | | | | | | |
| 674 | Method | CTC | | HATU | MITS | HATU | HATU | | HATU | HATU |
| | Building | Aib | | Phe0058 | MeOH | L | NMeK | | cPrG | AA0011 |
| | Block | | | | | | | | | |
| 675 | Method | CTC | | HATU | MITS | HATU | HATU | | HATU | HATU |
| | Building | Aib | | Phe0060 | MeOH | L | NMeK | | cPrG | AA0011 |
| | Block | | | | | | | | | |
| 676 | Method | CTC | | HATU | MITS | HATU | HATU | | HATU | HATUnf |
| | Building | Aib | | 2H165ClF | MeOH | L | NMeKDde | | cPrG | Acd0799 |
| | Block | | | | | | | | | |
| 677 | Method | CTC | | HATU | MITS | HATU | HATU | | HATU | HATU |
| | Building | Aib | | 2H105ClF | MeOH | L | NMeK | | KDde | AA0011 |
| | Block | | | | | | | | | |
| 678 | Method | CTC | | HATU | MITS | HATU | HATU | | HATU | HATU |
| | Building | Aib | | 2H105ClF | MeOH | L | NMeK | | KDde | AA0011 |
| | Block | | | | | | | | | |
| 679 | Method | CTC | | HATU | MITS | HATU | HATU | | HATU | HATU |
| | Building | Aib | | 2H105ClF | MeOH | L | NMeK | | KDde | AA0011 |
| | Block | | | | | | | | | |
| 680 | Method | CTC | | HATU | MITS | HATU | HATU | MITS | HATU | HATU |
| | Building | Acpc | | 25Clf | MeOH | 1 | kBoc | MeOH | cba | tFp |
| | Block | | | | | | | | | |

TABLE 2B
Building Blocks used and Procedures for Compound Preparation (Part 2)

| Ex. | Type | T10 | T11 | T12 | T13 | T14 | T15 | T16 | T17 | T18 |
|---|---|---|---|---|---|---|---|---|---|---|
| 1 | Method | HATUnf | | | | | | 20% | T3P Method A | |
| | | | | | | | | TFA | | |
| | Building | Acd317 | | | | | | | | |
| | Block | | | | | | | | | |
| 2 | Method | HATUnf | | | | | | 20% | T3P Method A | |
| | | | | | | | | TFA | | |
| | Building | Acd0423 | | | | | | | | |
| | Block | | | | | | | | | |
| 3 | Method | HATUnf | | DdeR | | Morph | | 20% | T3P Method A | |
| | | | | | | | | TFA | | |
| | Building | Acd0401 | | | | B2BE | | | | |
| | Block | | | | | | | | | |
| 4 | Method | HATUnf | | | | | | 20% | T3P Method A | |
| | | | | | | | | TFA | | |
| | Building | Acd0317 | | | | | | | | |
| | Block | | | | | | | | | |
| 5 | Method | HATUnf | | DdeR | | Morph | | 20% | T3P Method A | |
| | | | | | | | | TFA | | |
| | Building | Acd0401 | | | | B2BE | | | | |
| | Block | | | | | | | | | |
| 6 | Method | HATUnf | | | | | | 20% | T3P Method A | |
| | | | | | | | | TFA | | |
| | Building | Acd0401 | | | | | | | | |
| | Block | | | | | | | | | |
| 7 | Method | HATUnf | | DdeR | | Morph | | 20% | T3P Method A | |
| | | | | | | | | TFA | | |
| | Building | Acd0401 | | | | B2BE | | | | |
| | Block | | | | | | | | | |
| 8 | Method | HATUnf | | | | | | 20% | T3P Method A | |
| | | | | | | | | TFA | | |
| | Building | Acd0401 | | | | | | | | |
| | Block | | | | | | | | | |
| 9 | Method | HATUnf | | DdeR | | | Morph | 20% | T3P Method A | |
| | | | | | | | | TFA | | |
| | Building | Acd0401 | | | | | B2BE | | | |
| | Block | | | | | | | | | |
| 10 | Method | HATUnf | | DdeR | | Morph | | 20% | T3P Method A | |
| | | | | | | | | TFA | | |
| | Building | Acd0401 | | | | B2BE | | | | |
| | Block | | | | | | | | | |
| 11 | Method | HATUnf | | DdeR | | Morph | | 20% | T3P Method A | |
| | | | | | | | | TFA | | |
| | Building | Acd0401 | | | | B2BE | | | | |
| | Block | | | | | | | | | |
| 12 | Method | HATU | | DdeR | | Morph | | 20% | T3P Method A | |
| | | | | | | | | TFA | | |
| | Building | Acd0401 | | | | B2BE | | | | |
| | Block | | | | | | | | | |
| 13 | Method | HATU | | DdeR | | Morph | | 20% | T3P Method A | |
| | | | | | | | | TFA | | |
| | Building | Acd0401 | | | | B2BE | | | | |
| | Block | | | | | | | | | |
| 14 | Method | HATU | | DdeR | | Morph | | 20% | T3P Method A | |
| | | | | | | | | TFA | | |
| | Building | Acd0401 | | | | B2BE | | | | |
| | Block | | | | | | | | | |
| 15 | Method | HATUnf | | DdeR | | Morph | | 20% | T3P Method A | |
| | | | | | | | | TFA | | |
| | Building | Acd0401 | | | | B2BE | | | | |
| | Block | | | | | | | | | |
| 16 | Method | HATUnf | | DdeR | | Morph | | 20% | T3P Method A | |
| | | | | | | | | TFA | | |
| | Building | Acd0401 | | | | B2BE | | | | |
| | Block | | | | | | | | | |
| 17 | Method | HATUnf | | DdeR | | Morph | | 20% | T3P Method B | |
| | | | | | | | | TFA | | |
| | Building | Acd0401 | | | | B2BE | | | | |
| | Block | | | | | | | | | |
| 18 | Method | HATUnf | | DdeR | | | Morph | 20% | T3P Method A | |
| | | | | | | | | TFA | | |
| | Building | Acd0401 | | | | | B2BE | | | |
| | Block | | | | | | | | | |
| 19 | Method | HATUnf | | DdeR | | | Morph | 20% | T3P Method A | |
| | | | | | | | | TFA | | |
| | Building | Acd0401 | | | | | B2BE | | | |
| | Block | | | | | | | | | |
| 20 | Method | HATUnf | | DdeR | | Morph | Morph | 20% | T3P Method A | |
| | | | | | | | | TFA | | |
| | Building | Acd0401 | | | | B2BE | B2BE | | | |
| | Block | | | | | | | | | |
| 21 | Method | HATU | | DdeR | | Morph | | 20% | T3P Method A | |
| | | | | | | | | TFA | | |
| | Building | Acd0401 | | | | B2BE | | | | |
| | Block | | | | | | | | | |
| 22 | Method | HATU | | DdeR | | Morph | | 20% | T3P Method A | |
| | | | | | | | | TFA | | |
| | Building | Acd0401 | | | | B2BE | | | | |
| | Block | | | | | | | | | |
| 23 | Method | HATU | | DdeR | | Morph | | 20% | T3P Method A | |
| | | | | | | | | TFA | | |
| | Building | Acd0317 | | | | B2BE | | | | |
| | Block | | | | | | | | | |
| 24 | Method | HATU | | DdeR | | Morph | | 20% | T3P Method A | |
| | | | | | | | | TFA | | |
| | Building | Acd0423 | | | | B2BE | | | | |
| | Block | | | | | | | | | |
| 25 | Method | HATUnf | | DdeR | | Morph | | 20% | T3P Method A | |
| | | | | | | | | TFA | | |
| | Building | Acd0401 | | | | B2BE | | | | |
| | Block | | | | | | | | | |
| 26 | Method | HATUnf | | DdeR | | Morph | | 20% | T3P Method A | |
| | | | | | | | | TFA | | |
| | Building | Acd0401 | | | | B2BE | | | | |
| | Block | | | | | | | | | |
| 27 | Method | HATUnf | | | | | | 20% | T3P Method A | |
| | | | | | | | | TFA | | |
| | Building | Acd0401 | | | | | | | | |
| | Block | | | | | | | | | |
| 28 | Method | HATUnf | | | | | | 20% | T3P Method A | |
| | | | | | | | | TFA | | |
| | Building | Acd0317 | | | | | | | | |
| | Block | | | | | | | | | |
| 29 | Method | HATUnf | | | | | | 90% | PyBop Method | |
| | | | | | | | | TFA | B | |
| | Building | Acd0423 | | | | | | | | |
| | Block | | | | | | | | | |
| 30 | Method | HATUnf | | | | | | 20% | T3P Method A | |
| | | | | | | | | TFA | | |
| | Building | Acd0423 | | | | | | | | |
| | Block | | | | | | | | | |
| 31 | Method | HATUnf | | | | | | 20% | T3P Method A | |
| | | | | | | | | TFA | | |
| | Building | Acd0423 | | | | | | | | |
| | Block | | | | | | | | | |
| 32 | Method | HATUnf | | | | | | 20% | T3P Method A | |
| | | | | | | | | TFA | | |
| | Building | Acd0401 | | | | | | | | |
| | Block | | | | | | | | | |
| 33 | Method | HATU | | | | | | 20% | T3P Method A | |
| | | | | | | | | TFA | | |
| | Building | Acd0401 | | | | | | | | |
| | Block | | | | | | | | | |
| 34 | Method | HATU | | | | | | 20% | T3P Method A | |
| | | | | | | | | TFA | | |
| | Building | Acd0498 | | | | | | | | |
| | Block | | | | | | | | | |
| 35 | Method | HATUnf | | | | | | 20% | T3P Method A | |
| | | | | | | | | TFA | | |
| | Building | Acd0436 | | | | | | | | |
| | Block | | | | | | | | | |
| 36 | Method | HATUnf | | | | | | 20% | T3P Method A | |
| | | | | | | | | TFA | | |
| | Building | Acd0438 | | | | | | | | |
| | Block | | | | | | | | | |
| 37 | Method | HATUnf | | | | | | 20% | T3P Method A | |
| | | | | | | | | TFA | | |
| | Building | Acd0317 | | | | | | | | |
| | Block | | | | | | | | | |
| 38 | Method | HATUnf | | | | | | 20% | T3P Method A | |
| | | | | | | | | TFA | | |
| | Building | Acd0503 | | | | | | | | |
| | Block | | | | | | | | | |
| 39 | Method | HATUnf | | | | | | 20% | T3P Method A | |
| | | | | | | | | TFA | | |
| | Building | Acd0504 | | | | | | | | |
| | Block | | | | | | | | | |
| 40 | Method | HATUnf | | | | | | 20% | T3P Method A | |
| | | | | | | | | TFA | | |
| | Building | Acd0401 | | | | | | | | |
| | Block | | | | | | | | | |
| 41 | Method | HATUnf | | | | | | 20% | PyBop Method | |
| | | | | | | | | TFA | B | |
| | Building | Acd0423 | | | | | | | | |
| | Block | | | | | | | | | |
| 42 | Method | HATUnf | | | | | | 30% | T3P Method A | |
| | | | | | | | | TFA | | |
| | Building | Acd0423 | | | | | | | | |
| | Block | | | | | | | | | |
| 43 | Method | HATUnf | | | | | | 30% | T3P Method A | |
| | | | | | | | | TFA | | |
| | Building | Acd0486 | | | | | | | | |
| | Block | | | | | | | | | |
| 44 | Method | HATUnf | | | | | | 30% | T3P Method A | |
| | | | | | | | | TFA | | |
| | Building | Acd0423 | | | | | | | | |
| | Block | | | | | | | | | |
| 45 | Method | HATUnf | | | | | | 30% | T3P Method A | |
| | | | | | | | | TFA | | |
| | Building | Acd0486 | | | | | | | | |
| | Block | | | | | | | | | |
| 46 | Method | HATUnf | | | | | | 20% | T3P Method A | |
| | | | | | | | | TFA | | |
| | Building | Acd0423 | | | | | | | | |
| | Block | | | | | | | | | |
| 47 | Method | HATUnf | | | | | | 20% | T3P Method A | |
| | | | | | | | | TFA | | |
| | Building | Acd0423 | | | | | | | | |
| | Block | | | | | | | | | |
| 48 | Method | HATUnf | | | | | | 20% | T3P Method A | |
| | | | | | | | | TFA | | |
| | Building | Acd0423 | | | | | | | | |
| | Block | | | | | | | | | |
| 49 | Method | HATUnf | | | | | | 20% | PyBop Method | |
| | | | | | | | | TFA | B | |
| | Building | Acd0486 | | | | | | | | |
| | Block | | | | | | | | | |
| 50 | Method | HATUnf | | | | | | 20% | PyBop Method | |
| | | | | | | | | TFA | B | |
| | Building | Acd0486 | | | | | | | | |
| | Block | | | | | | | | | |
| 51 | Method | HATUnf | | | | | | 20% | T3P Method B | |
| | | | | | | | | TFA | | |
| | Building | Acd0423 | | | | | | | | |
| | Block | | | | | | | | | |
| 52 | Method | HATUnf | | | | | | 20% | T3P Method A | |
| | | | | | | | | TFA | | |
| | Building | Acd0423 | | | | | | | | |
| | Block | | | | | | | | | |
| 53 | Method | HATUnf | | | | | | 20% | T3P Method A | |
| | | | | | | | | TFA | | |
| | Building | Acd0401 | | | | | | | | |
| | Block | | | | | | | | | |
| 54 | Method | HATUnf | | | | | | 20% | T3P Method A | |
| | | | | | | | | TFA | | |
| | Building | Acd0401 | | | | | | | | |
| | Block | | | | | | | | | |
| 55 | Method | HATUnf | | | | | | 20% | PyBop Method | |
| | | | | | | | | TFA | B | |
| | Building | Acd0423 | | | | | | | | |
| | Block | | | | | | | | | |
| 56 | Method | HATUnf | | | | | | 20% | T3P Method A | |
| | | | | | | | | TFA | | |
| | Building | Acd0503 | | | | | | | | |
| | Block | | | | | | | | | |
| 57 | Method | | | | | | | 20% | T3P Method A | |
| | | | | | | | | TFA | | |
| | Building | | | | | | | | | |
| | Block | | | | | | | | | |
| 58 | Method | HATUnf | | | | | | 20% | T3P Method A | |
| | | | | | | | | TFA | | |
| | Building | Acd0504 | | | | | | | | |
| | Block | | | | | | | | | |
| 59 | Method | HATUnf | | DdeR | | Morph | | 20% | T3P Method A | |
| | | | | | | | | TFA | | |
| | Building | Acd0504 | | | | B2BE | | | | |
| | Block | | | | | | | | | |
| 60 | Method | HATUnf | | DdeR | | Morph | | 20% | T3P Method A | |
| | | | | | | | | TFA | | |
| | Building | Acd0504 | | | | B2BE | | | | |
| | Block | | | | | | | | | |
| 61 | Method | HATUnf | | | | | | 20% | T3P Method A | |
| | | | | | | | | TFA | | |
| | Building | Acd0401 | | | | | | | | |
| | Block | | | | | | | | | |
| 62 | Method | HATUnf | | | | | | 20% | T3P Method A | |
| | | | | | | | | TFA | | |
| | Building | Acd0504 | | | | | | | | |
| | Block | | | | | | | | | |
| 63 | Method | HATUnf | | | | | | 20% | T3P Method B | |
| | | | | | | | | TFA | | |
| | Building | Acd0504 | | | | | | | | |
| | Block | | | | | | | | | |
| 64 | Method | HATUnf | | | | | | 20% | PyBop Method | |
| | | | | | | | | TFA | B | |
| | Building | Acd0486 | | | | | | | | |
| | Block | | | | | | | | | |
| 65 | Method | HATUnf | | | | | | 20% | PyBop Method | |
| | | | | | | | | TFA | B | |
| | Building | Acd0486 | | | | | | | | |
| | Block | | | | | | | | | |
| 66 | Method | HATUnf | | | | | | 20% | PyBop Method | |
| | | | | | | | | TFA | B | |
| | Building | Acd0504 | | | | | | | | |
| | Block | | | | | | | | | |
| 67 | Method | HATUnf | | | | | | 20% | T3P Method A | |
| | | | | | | | | TFA | | |
| | Building | Acd0504 | | | | | | | | |
| | Block | | | | | | | | | |
| 68 | Method | HATUnf | | | | | | 20% | T3P Method A | |
| | | | | | | | | TFA | | |
| | Building | Acd0504 | | | | | | | | |
| | Block | | | | | | | | | |
| 69 | Method | HATUnf | | | | | | 20% | T3P Method A | |
| | | | | | | | | TFA | | |
| | Building | Acd0504 | | | | | | | | |
| | Block | | | | | | | | | |
| 70 | Method | HATUnf | | | | | | 20% | T3P Method A | |
| | | | | | | | | TFA | | |
| | Building | Acd0504 | | | | | | | | |
| | Block | | | | | | | | | |
| 71 | Method | HATUnf | | | | | | 20% | T3P Method A | |
| | | | | | | | | TFA | | |
| | Building | Acd0504 | | | | | | | | |
| | Block | | | | | | | | | |
| 72 | Method | | | | | | | 20% | T3P Method A | |
| | | | | | | | | TFA | | |
| | Building | | | | | | | | | |
| | Block | | | | | | | | | |
| 73 | Method | KO | | | | | | 20% | T3P Method A | |
| | | | | | | | | TFA | | |
| | Building | Acd0504 | | | | | | | | |
| | Block | | | | | | | | | |
| 74 | Method | KO | | | | | | 20% | T3P Method A | |
| | | | | | | | | TFA | | |
| | Building | Acd0504 | | | | | | | | |
| | Block | | | | | | | | | |
| 75 | Method | KO | | | | | | 20% | T3P Method A | |
| | | | | | | | | TFA | | |
| | Building | Acd0533 | | | | | | | | |
| | Block | | | | | | | | | |
| 76 | Method | KO | | | | | | 20% | T3P Method B | |
| | | | | | | | | TFA | | |
| | Building | Acd0504 | | | | | | | | |
| | Block | | | | | | | | | |
| 77 | Method | KO | | | | | | 20% | T3P Method B | |
| | | | | | | | | TFA | | |
| | Building | Acd0504 | | | | | | | | |
| | Block | | | | | | | | | |
| 78 | Method | KO | | | | | | 20% | T3P Method A | |
| | | | | | | | | TFA | | |
| | Building | Acd0504 | | | | | | | | |
| | Block | | | | | | | | | |
| 79 | Method | HATUnf | | | | | | 20% | PyBop Method | |
| | | | | | | | | TFA | B | |
| | Building | Acd0486 | | | | | | | | |
| | Block | | | | | | | | | |
| 80 | Method | KO | | | | | | 20% | T3P Method A | |
| | | | | | | | | TFA | | |
| | Building | Acd0504 | | | | | | | | |
| | Block | | | | | | | | | |
| 81 | Method | | | | | | | 20% | T3P Method A | |
| | | | | | | | | TFA | | |
| | Building | | | | | | | | | |
| | Block | | | | | | | | | |
| 82 | Method | KO | | | | | | 20% | T3P Method A | |
| | | | | | | | | TFA | | |
| | Building | Acd0504 | | | | | | | | |
| | Block | | | | | | | | | |
| 83 | Method | HATUnf | | | | | | 20% | PyBop Method | |
| | | | | | | | | TFA | B | |
| | Building | Acd0486 | | | | | | | | |
| | Block | | | | | | | | | |
| 84 | Method | HATUnf | | | | | | 20% | T3P Method A | |
| | | | | | | | | TFA | | |
| | Building | Acd0520 | | | | | | | | |
| | Block | | | | | | | | | |
| 85 | Method | HATUnf | | | | | | 20% | T3P Method A | |
| | | | | | | | | TFA | | |
| | Building | Acd0505 | | | | | | | | |
| | Block | | | | | | | | | |
| 86 | Method | HATUnf | | | | | | 20% | T3P Method A | |
| | | | | | | | | TFA | | |
| | Building | Acd0504 | | | | | | | | |
| | Block | | | | | | | | | |
| 87 | Method | KO | | | | | | 20% | T3P Method A | |
| | | | | | | | | TFA | | |
| | Building | Acd0504 | | | | | | | | |
| | Block | | | | | | | | | |
| 88 | Method | HATUnf | | | | | | 20% | T3P Method A | |
| | | | | | | | | TFA | | |
| | Building | Acd0436 | | | | | | | | |
| | Block | | | | | | | | | |
| 89 | Method | HATUnf | | | | | | 20% | T3P Method A | |
| | | | | | | | | TFA | | |
| | Building | Acd0532 | | | | | | | | |
| | Block | | | | | | | | | |
| 90 | Method | HATUnf | | | | | | 20% | PyBop Method | |
| | | | | | | | | TFA | B | |
| | Building | Acd0486 | | | | | | | | |
| | Block | | | | | | | | | |
| 91 | Method | HATUnf | | | | | | 20% | T3P Method A | |
| | | | | | | | | TFA | | |
| | Building | Acd0504 | | | | | | | | |
| | Block | | | | | | | | | |
| 92 | Method | HATUnf | | | | | | 20% | T3P Method A | |
| | | | | | | | | TFA | | |
| | Building | Acd0504 | | | | | | | | |
| | Block | | | | | | | | | |
| 93 | Method | HATUnf | | | | | | 20% | T3P Method A | |
| | | | | | | | | TFA | | |
| | Building | Acd0504 | | | | | | | | |
| | Block | | | | | | | | | |
| 94 | Method | HATUnf | | | | | | 20% | T3P Method A | |
| | | | | | | | | TFA | | |
| | Building | Acd0504 | | | | | | | | |
| | Block | | | | | | | | | |
| 95 | Method | KO | | | | | | 20% | T3P Method A | |
| | | | | | | | | TFA | | |
| | Building | Acd0504 | | | | | | | | |
| | Block | | | | | | | | | |
| 96 | Method | KO | | | | | | 20% | T3P Method A | |
| | | | | | | | | TFA | | |
| | Building | Acd0504 | | | | | | | | |
| | Block | | | | | | | | | |
| 97 | Method | HATUnf | | | | | | 20% | T3P Method B | |
| | | | | | | | | TFA | | |
| | Building | Acd0504 | | | | | | | | |
| | Block | | | | | | | | | |
| 98 | Method | HATUnf | | | | | | 20% | T3P Method B | |
| | | | | | | | | TFA | | |
| | Building | Acd0503 | | | | | | | | |
| | Block | | | | | | | | | |
| 99 | Method | | | | | | | 20% | T3P Method A | |
| | | | | | | | | TFA | | |
| | Building | | | | | | | | | |
| | Block | | | | | | | | | |
| 100 | Method | HATUnf | | | | | | 20% | T3P Method A | |
| | | | | | | | | TFA | | |
| | Building | Acd0504 | | | | | | | | |
| | Block | | | | | | | | | |
| 101 | Method | HATUnf | | DdeR | | Morph | | 20% | T3P Method A | |
| | | | | | | | | TFA | | |
| | Building | Acd0525 | | | | B2BE | | | | |
| | Block | | | | | | | | | |
| 102 | Method | HATUnf | | DdeR | | Morph | | 20% | T3P Method A | |
| | | | | | | | | TFA | | |
| | Building | Acd0525 | | | | B2BE | | | | |
| | Block | | | | | | | | | |
| 103 | Method | HATUnf | | DdeR | | | Morph | 20% | T3P Method A | |
| | | | | | | | | TFA | | |
| | Building | Acd0401 | | | | | B2BE | | | |
| | Block | | | | | | | | | |
| 104 | Method | | | | | | | 20% | T3P Method A | |
| | | | | | | | | TFA | | |
| | Building | | | | | | | | | |
| | Block | | | | | | | | | |
| 105 | Method | HATUnf | | | | | | 20% | T3P Method A | |
| | | | | | | | | TFA | | |
| | Building | Acd0504 | | | | | | | | |
| | Block | | | | | | | | | |
| 106 | Method | | | | | | | 20% | PyBop Method | |
| | | | | | | | | TFA | B | |
| | Building | | | | | | | | | |
| | Block | | | | | | | | | |
| 107 | Method | HATUnf | | | | | | 20% | T3P Method A | |
| | | | | | | | | TFA | | |
| | Building | Acd0504 | | | | | | | | |
| | Block | | | | | | | | | |
| 108 | Method | | | | | | | 20% | T3P Method A | |
| | | | | | | | | TFA | | |
| | Building | | | | | | | | | |
| | Block | | | | | | | | | |
| 109 | Method | HATUnf | | | | | | 20% | T3P Method A | |
| | | | | | | | | TFA | | |
| | Building | Acd0504 | | | | | | | | |
| | Block | | | | | | | | | |
| 110 | Method | HATUnf | | | | | | 20% | T3P Method B | |
| | | | | | | | | TFA | | |
| | Building | Acd0504 | | | | | | | | |
| | Block | | | | | | | | | |
| 111 | Method | KO | | | | | | \|20% | T3P Method A | |
| | | | | | | | | TFA | | |
| | Building | Acd0504 | | | | | | | | |
| | Block | | | | | | | | | |
| 112 | Method | KO | | | | | | 20% | T3P Method B | |
| | | | | | | | | TFA | | |
| | Building | Acd0317 | | | | | | | | |
| | Block | | | | | | | | | |
| 113 | Method | KO | | | | | | 20% | PyBop Method | |
| | | | | | | | | TFA | B | |
| | Building | Acd0486 | | | | | | | | |
| | Block | | | | | | | | | |
| 114 | Method | | | | | | | 20% | T3P Method A | |
| | | | | | | | | TFA | | |
| | Building | | | | | | | | | |
| | Block | | | | | | | | | |
| 115 | Method | HATUnf | | | | | | 20% | T3P Method B | |
| | | | | | | | | TFA | | |
| | Building | Acd0504 | | | | | | | | |
| | Block | | | | | | | | | |
| 116 | Method | | | | | | | 20% | T3P Method B | |
| | | | | | | | | TFA | | |
| | Building | | | | | | | | | |
| | Block | | | | | | | | | |
| 117 | Method | KO | | | | | | 20% | PyBop Method | |
| | | | | | | | | TFA | B | |
| | Building | Acd0486 | | | | | | | | |
| | Block | | | | | | | | | |
| 118 | Method | | | | | | | 20% | T3P Method A | |
| | | | | | | | | TFA | | |
| | Building | | | | | | | | | |
| | Block | | | | | | | | | |
| 119 | Method | | | | | | | 20% | T3P Method A | |
| | | | | | | | | TFA | | |
| | Building | | | | | | | | | |
| | Block | | | | | | | | | |
| 120 | Method | KO | | | | | | 20% | PyBop Method | |
| | | | | | | | | TFA | B | |
| | Building | Acd0486 | | | | | | | | |
| | Block | | | | | | | | | |
| 121 | Method | HATUnf | | | | | | 20% | T3P Method A | |
| | | | | | | | | TFA | | |
| | Building | Acd0504 | | | | | | | | |
| | Block | | | | | | | | | |
| 122 | Method | HATU | | DdeR | MITS | | | 20% | T3P Method A | |
| | | | | | | | | HFIP | | |
| | Building | Acd0504 | | | MeOH | | | | | |
| | Block | | | | | | | | | |
| 123 | Method | HATUnf | | | | | | 20% | T3P Method B | |
| | | | | | | | | TFA | | |
| | Building | Acd0525 | | | | | | | | |
| | Block | | | | | | | | | |
| 124 | Method | HATUnf | | | | | | 20% | T3P Method B | |
| | | | | | | | | TFA | | |
| | Building | Acd0525 | | | | | | | | |
| | Block | | | | | | | | | |
| 125 | Method | HATUnf | | | | | | 20% | T3P Method A | |
| | | | | | | | | TFA | | |
| | Building | Acd0504 | | | | | | | | |
| | Block | | | | | | | | | |
| 126 | Method | HATUnf | | | | | | 20% | T3P Method B | |
| | | | | | | | | TFA | | |
| | Building | Acd0504 | | | | | | | | |
| | Block | | | | | | | | | |
| 127 | Method | HATU | | DdeR | | Morph | | 20% | T3P Method A | |
| | | | | | | | | TFA | | |
| | Building | Acd0504 | | | | B2BE | | | | |
| | Block | | | | | | | | | |
| 128 | Method | | | | | | | 20% | T3P Method A | |
| | | | | | | | | TFA | | |
| | Building | | | | | | | | | |
| | Block | | | | | | | | | |
| 129 | Method | | | | | | | 20% | T3P Method B | |
| | | | | | | | | TFA | | |
| | Building | | | | | | | | | |
| | Block | | | | | | | | | |
| 130 | Method | HATUnf | | | | | | 20% | PyBop Method | |
| | | | | | | | | TFA | B | |
| | Building | Acd0486 | | | | | | | | |
| | Block | | | | | | | | | |
| 131 | Method | HATUnf | | | | | | 20% | PyBop Method | |
| | | | | | | | | TFA | B | |
| | Building | Acd0486 | | | | | | | | |
| | Block | | | | | | | | | |
| 132 | Method | HATUnf | | | | | | 20% | T3P Method B | |
| | | | | | | | | TFA | | |
| | Building | Acd0504 | | | | | | | | |
| | Block | | | | | | | | | |
| 133 | Method | KO | | DdeR | | HATU | | 20% | T3P Method A | |
| | | | | | | | | TFA | | |
| | Building | Acd0504 | | | | RA230 | | | | |
| | Block | | | | | | | | | |
| 134 | Method | | | DdeR | | HATU | | 20% | T3P Method A | |
| | | | | | | | | TFA | | |
| | Building | | | | | RA230 | | | | |
| | Block | | | | | | | | | |
| 135 | Method | HATUnf | | DdeR | | HATU | | 20% | PyBop Method | |
| | | | | | | | | TFA | B | |
| | Building | Acd0486 | | | | RA230 | | | | |
| | Block | | | | | | | | | |
| 136 | Method | HATUnf | | DdeR | | HATU | | 20% | PyBop Method | |
| | | | | | | | | TFA | B | |
| | Building | Acd0486 | | | | RA230 | | | | |
| | Block | | | | | | | | | |
| 137 | Method | KO | | | | | | 20% | T3P Method A | |
| | | | | | | | | TFA | | |
| | Building | Acd0504 | | | | | | | | |
| | Block | | | | | | | | | |
| 138 | Method | HATUnf | | | | | | 20% | T3P Method A | |
| | | | | | | | | TFA | | |
| | Building | Acd0504 | | | | | | | | |
| | Block | | | | | | | | | |
| 139 | Method | HATUnf | | | | | | 20% | PyBop Method | |
| | | | | | | | | TFA | B | |
| | Building | Acd0486 | | | | | | | | |
| | Block | | | | | | | | | |
| 140 | Method | HATUnf | | | | | | 20% | PyBop Method | |
| | | | | | | | | TFA | B | |
| | Building | Acd0486 | | | | | | | | |
| | Block | | | | | | | | | |
| 141 | Method | HATUnf | | | | | | 20% | PyBop Method | |
| | | | | | | | | TFA | B | |
| | Building | Acd0486 | | | | | | | | |
| | Block | | | | | | | | | |
| 142 | Method | HATUnf | | | | | | 20% | PyBop Method | |
| | | | | | | | | TFA | B | |
| | Building | Acd0486 | | | | | | | | |
| | Block | | | | | | | | | |
| 143 | Method | HATUnf | | | | | | 20% | PyBop Method | |
| | | | | | | | | TFA | B | |
| | Building | Acd0486 | | | | | | | | |
| | Block | | | | | | | | | |
| 144 | Method | HATUnf | | | | | | 20% | PyBop Method | |
| | | | | | | | | TFA | B | |
| | Building | Acd0486 | | | | | | | | |
| | Block | | | | | | | | | |
| 145 | Method | HATUnf | | | | | | 20% | PyBop Method | |
| | | | | | | | | TFA | B | |
| | Building | Acd0486 | | | | | | | | |
| | Block | | | | | | | | | |
| 146 | Method | KO | | | | | | 20% | T3P Method A | |
| | | | | | | | | TFA | | |
| | Building | Acd0533 | | | | | | | | |
| | Block | | | | | | | | | |
| 147 | Method | HATUnf | | | | | | 20% | T3P Method A | |
| | | | | | | | | TFA | | |
| | Building | Acd0532 | | | | | | | | |
| | Block | | | | | | | | | |
| 148 | Method | HATUnf | | DdeR | | | | 24% | T3P Method A | |
| | | | | | | | | HFIP | | |
| | Building | Acd0532 | | | | | | | | |
| | Block | | | | | | | | | |
| 149 | Method | KO | | | | | | 20% | T3P Method A | |
| | | | | | | | | TFA | | |
| | Building | Acd0533 | | | | | | | | |
| | Block | | | | | | | | | |
| 150 | Method | | | | | | | 20% | T3P Method A | |
| | | | | | | | | TFA | | |
| | Building | | | | | | | | | |
| | Block | | | | | | | | | |
| 151 | Method | HATUnf | | | | | | 20% | T3P Method A | |
| | | | | | | | | TFA | | |
| | Building | Acd0532 | | | | | | | | |
| | Block | | | | | | | | | |
| 152 | Method | HATUnf | | | | | | 20% | T3P Method A | |
| | | | | | | | | TFA | | |
| | Building | Acd0532 | | | | | | | | |
| | Block | | | | | | | | | |
| 153 | Method | HATUnf | | | | | | 20% | T3P Method A | |
| | | | | | | | | TFA | | |
| | Building | Acd0503 | | | | | | | | |
| | Block | | | | | | | | | |
| 154 | Method | HATUnf | | | | | | 20% | T3P Method A | |
| | | | | | | | | TFA | | |
| | Building | Acd0505 | | | | | | | | |
| | Block | | | | | | | | | |
| 155 | Method | HATUnf | | | | | | 20% | T3P Method A | |
| | | | | | | | | TFA | | |
| | Building | Acd0423 | | | | | | | | |
| | Block | | | | | | | | | |
| 156 | Method | HATUnf | | | | | | 20% | T3P Method A | |
| | | | | | | | | TFA | | |
| | Building | Acd0540 | | | | | | | | |
| | Block | | | | | | | | | |
| 157 | Method | KO | | | | | | 20% | T3P Method A | |
| | | | | | | | | TFA | | |
| | Building | Acd0542 | | | | | | | | |
| | Block | | | | | | | | | |
| 158 | Method | HATUnf | | | | | | 20% | PyBop Method | |
| | | | | | | | | TFA | B | |
| | Building | Acd0544 | | | | | | | | |
| | Block | | | | | | | | | |
| 159 | Method | HATUnf | | | | | | 20% | T3P Method A | |
| | | | | | | | | TFA | | |
| | Building | Acd0317 | | | | | | | | |
| | Block | | | | | | | | | |
| 160 | Method | HATUnf | | | | | | 20% | T3P Method A | |
| | | | | | | | | TFA | | |
| | Building | Acd0504 | | | | | | | | |
| | Block | | | | | | | | | |
| 161 | Method | HATUnf | | | | | | 20% | T3P Method A | |
| | | | | | | | | TFA | | |
| | Building | Acd0504 | | | | | | | | |
| | Block | | | | | | | | | |
| 162 | Method | HATUnf | | | | | | 20% | T3P Method A | |
| | | | | | | | | TFA | | |
| | Building | Acd0504 | | | | | | | | |
| | Block | | | | | | | | | |
| 163 | Method | HATUnf | | | | | | 20% | PyBop Method | |
| | | | | | | | | TFA | B | |
| | Building | Acd0486 | | | | | | | | |
| | Block | | | | | | | | | |
| 164 | Method | HATUnf | | | | | | 20% | PyBop Method | |
| | | | | | | | | TFA | B | |
| | Building | Acd0486 | | | | | | | | |
| | Block | | | | | | | | | |
| 165 | Method | KO | | | | | | 20% | T3P Method A | |
| | | | | | | | | TFA | | |
| | Building | Acd0504 | | | | | | | | |
| | Block | | | | | | | | | |
| 166 | Method | | | | | | | 20% | T3P Method A | |
| | | | | | | | | TFA | | |
| | Building | | | | | | | | | |
| | Block | | | | | | | | | |
| 167 | Method | HATUnf | | | | | | 20% | T3P Method A | |
| | | | | | | | | TFA | | |
| | Building | Acd0505 | | | | | | | | |
| | Block | | | | | | | | | |
| 168 | Method | HATUnf | | | | | | 20% | PyBop Method | |
| | | | | | | | | TFA | B | |
| | Building | Acd0486 | | | | | | | | |
| | Block | | | | | | | | | |
| 169 | Method | HATUnf | | | | | | 20% | PyBop Method | |
| | | | | | | | | TFA | B | |
| | Building | Acd0486 | | | | | | | | |
| | Block | | | | | | | | | |
| 170 | Method | HATUnf | | | | | | 20% | PyBop Method | |
| | | | | | | | | TFA | B | |
| | Building | Acd0486 | | | | | | | | |
| | Block | | | | | | | | | |
| 171 | Method | HATUnf | | | | | | 20% | T3P Method A | |
| | | | | | | | | TFA | | |
| | Building | Acd0504 | | | | | | | | |
| | Block | | | | | | | | | |
| 172 | Method | HATUnf | | | | | | 20% | T3P Method B | |
| | | | | | | | | TFA | | |
| | Building | Acd0504 | | | | | | | | |
| | Block | | | | | | | | | |
| 173 | Method | HATUnf | | | | | | \|20% | T3P Method B | |
| | | | | | | | | TFA | | |
| | Building | Acd0532 | | | | | | | | |
| | Block | | | | | | | | | |
| 174 | Method | HATUnf | | | | | | 20% | T3P Method A | |
| | | | | | | | | TFA | | |
| | Building | Acd0505 | | | | | | | | |
| | Block | | | | | | | | | |
| 175 | Method | HATUnf | | | | | | 20% | T3P Method A | |
| | | | | | | | | TFA | | |
| | Building | Acd0540 | | | | | | | | |
| | Block | | | | | | | | | |
| 176 | Method | HATUnf | | | | | | 20% | T3P Method B | |
| | | | | | | | | TFA | | |
| | Building | Acd0505 | | | | | | | | |
| | Block | | | | | | | | | |
| 177 | Method | HATUnf | | | | | | 20% | T3P Method B | |
| | | | | | | | | TFA | | |
| | Building | Acd0504 | | | | | | | | |
| | Block | | | | | | | | | |
| 178 | Method | HATUnf | | | | | | 20% | T3P Method B | |
| | | | | | | | | TFA | | |
| | Building | Acd0504 | | | | | | | | |
| | Block | | | | | | | | | |
| 179 | Method | HATUnf | | | | | | 20% | T3P Method B | |
| | | | | | | | | TFA | | |
| | Building | Acd0504 | | | | | | | | |
| | Block | | | | | | | | | |
| 180 | Method | HATUnf | | | | | | 20% | T3P Method B | |
| | | | | | | | | TFA | | |
| | Building | Acd0504 | | | | | | | | |
| | Block | | | | | | | | | |
| 181 | Method | HATUnf | | | | | | 20% | T3P Method B | |
| | | | | | | | | TFA | | |
| | Building | Acd0504 | | | | | | | | |
| | Block | | | | | | | | | |
| 182 | Method | HATUnf | | | | | | 20% | T3P Method B | |
| | | | | | | | | TFA | | |
| | Building | Acd0504 | | | | | | | | |
| | Block | | | | | | | | | |
| 183 | Method | HATUnf | | DdeR | | Morph | | 20% | T3P Method B | |
| | | | | | | | | TFA | | |
| | Building | Acd0504 | | | | B2BE | | | | |
| | Block | | | | | | | | | |
| 184 | Method | HATUnf | | | | | | 20% | PyBop Method | |
| | | | | | | | | TFA | B | |
| | Building | Acd0504 | | | | | | | | |
| | Block | | | | | | | | | |
| 185\| | Method | HATUnf | | | | | | 20% | PyBop Method | |
| | | | | | | | | TFA | B | |
| | Building | Acd0486 | | | | | | | | |
| | Block | | | | | | | | | |
| 186 | Method | HATUnf | | | | | | 20% | PyBop Method | |
| | | | | | | | | TFA | B | |
| | Building | Acd0486 | | | | | | | | |
| | Block | | | | | | | | | |
| 187 | \|Method | HATUnf | | | | | | 20% | PyBop Method | |
| | | | | | | | | TFA | B | |
| | Building | Acd0486 | | | | | | | | |
| | Block | | | | | | | | | |
| 188 | Method | HATUnf | | | | | | 20% | PyBop Method | |
| | | | | | | | | TFA | B | |
| | Building | Acd0486 | | | | | | | | |
| | Block | | | | | | | | | |
| 189 | Method | HATUnf | | | | | | 20% | PyBop Method | |
| | | | | | | | | TFA | B | |
| | Building | Acd0486 | | | | | | | | |
| | Block | | | | | | | | | |
| 190\| | Method | KO | | | | | | 20% | T3P Method A | |
| | | | | | | | | TFA | | |
| | Building | Acd0504 | | | | | | | | |
| | Block | | | | | | | | | |
| 191 | Method | HATUnf | | | | | | 20% | T3P Method B | |
| | | | | | | | | TFA | | |
| | Building | Acd0317 | | | | | | | | |
| | Block | | | | | | | | | |
| 192 | Method | HATUnf | | | | | | 20% | T3P Method A | |
| | | | | | | | | TFA | | |
| | Building | Acd0317 | | | | | | | | |
| | Block | | | | | | | | | |
| 193 | Method | HATUnf | | | | | | 20% | PyBop Method | |
| | | | | | | | | TFA | B | |
| | Building | Acd0486 | | | | | | | | |
| | Block | | | | | | | | | |
| 194 | Method | HATUnf | | | | | | 20% | PyBop Method | |
| | | | | | | | | TFA | B | |
| | Building | Acd0486 | | | | | | | | |
| | Block | | | | | | | | | |
| 195 | Method | HATUnf | | | | | | 20% | PyBop Method | |
| | | | | | | | | TFA | B | |
| | Building | Acd0486 | | | | | | | | |
| | Block | | | | | | | | | |
| 196 | Method | HATUnf | | | | | | 20% | PyBop Method | |
| | | | | | | | | TFA | B | |
| | Building | Acd0486 | | | | | | | | |
| | Block | | | | | | | | | |
| 197 | Method | HATUnf | | | | | | 20% | T3P Method A | |
| | | | | | | | | TFA | | |
| | Building | Acd0532 | | | | | | | | |
| | Block | | | | | | | | | |
| 198 | Method | | | | | | | 20% | T3P Method B | |
| | | | | | | | | TFA | | |
| | Building | | | | | | | | | |
| | Block | | | | | | | | | |
| 199 | Method | | | | | | | 20% | T3P Method B | |
| | | | | | | | | TFA | | |
| | Building | | | | | | | | | |
| | Block | | | | | | | | | |
| 200 | Method | HATUnf | | | | | | 20% | PyBop Method | |
| | | | | | | | | TFA | B | |
| | Building | Acd0486 | | | | | | | | |
| | Block | | | | | | | | | |
| 201 | Method | HATUnf | | | | | | 20% | PyBop Method | |
| | | | | | | | | TFA | B | |
| | Building | Acd0486 | | | | | | | | |
| | Block | | | | | | | | | |
| 202 | Method | HATUnf | | DdeR | | HATUnf | | 20% | PyBop Method | |
| | | | | | | | | TFA | B | |
| | Building | Acd0486 | | | | RA230 | | | | |
| | Block | | | | | | | | | |
| 203 | Method | KO | | | | | | 20% | T3P Method A | |
| | | | | | | | | TFA | | |
| | Building | Acd0532 | | | | | | | | |
| | Block | | | | | | | | | |
| 204 | Method | KO | | | | | | 20% | PyBop Method | |
| | | | | | | | | TFA | B | |
| | Building | Acd0532 | | | | | | | | |
| | Block | | | | | | | | | |
| 205 | Method | KO | | | | | | 20% | PyBop Method | |
| | | | | | | | | TFA | B | |
| | Building | Acd0532 | | | | | | | | |
| | Block | | | | | | | | | |
| 206 | Method | HATUnf | | | | | | 20% | PyBop Method | |
| | | | | | | | | TFA | B | |
| | Building | Acd0486 | | | | | | | | |
| | Block | | | | | | | | | |
| 207 | Method | HATUnf | | | | | | 20% | PyBop Method | |
| | | | | | | | | TFA | B | |
| | Building | Acd0486 | | | | | | | | |
| | Block | | | | | | | | | |
| 208 | Method | HATUnf | | | | | | 20% | PyBop Method | |
| | | | | | | | | TFA | B | |
| | Building | Acd0486 | | | | | | | | |
| | Block | | | | | | | | | |
| 209 | Method | HATUnf | | | | | | 20% | T3P Method B | |
| | | | | | | | | TFA | | |
| | Building | Acd0504 | | | | | | | | |
| | Block | | | | | | | | | |
| 210 | Method | HATUnf | | | | | | 20% | PyBop Method | |
| | | | | | | | | TFA | B | |
| | Building | Acd0486 | | | | | | | | |
| | Block | | | | | | | | | |
| 211 | Method | HATUnf | | | | | | 20% | T3P Method B | |
| | | | | | | | | TFA | | |
| | Building | Acd0503 | | | | | | | | |
| | Block | | | | | | | | | |
| 212 | Method | HATUnf | | | | | | 20% | T3P Method B | |
| | | | | | | | | TFA | | |
| | Building | Acd0503 | | | | | | | | |
| | Block | | | | | | | | | |
| 213 | Method | HATUnf | | | | | | 20% | T3P Method B | |
| | | | | | | | | TFA | | |
| | Building | Acd0505 | | | | | | | | |
| | Block | | | | | | | | | |
| 214 | Method | HATUnf | | | | | | 20% | PyBop Method | |
| | | | | | | | | TFA | B | |
| | Building | Acd0486 | | | | | | | | |
| | Block | | | | | | | | | |
| 215 | Method | KO | | | | | | 20% | T3P Method A | |
| | | | | | | | | TFA | | |
| | Building | Acd0573 | | | | | | | | |
| | Block | | | | | | | | | |
| 216 | Method | HATUnf | | DdeR | MITS | | | 20% | T3P Method A | |
| | | | | | | | | TFA | | |
| | Building | Acd0504 | | | CD3OD | | | | | |
| | Block | | | | | | | | | |
| 217 | Method | HATUnf | | | | | | 20% | T3P Method A | |
| | | | | | | | | TFA | | |
| | Building | Acd0504 | | | | | | | | |
| | Block | | | | | | | | | |
| 218 | Method | HATUnf | | DdeR | MITS | | | 20% | T3P Method A | |
| | | | | | | | | TFA | | |
| | Building | Acd0504 | | | CD3OD | | | | | |
| | Block | | | | | | | | | |
| 219 | Method | HATUnf | | | | | | 20% | T3P Method B | |
| | | | | | | | | TFA | | |
| | Building | Acd0317 | | | | | | | | |
| | Block | | | | | | | | | |
| 220 | Method | HATUnf | | | | | | 20% | T3P Method B | |
| | | | | | | | | TFA | | |
| | Building | Acd0540 | | | | | | | | |
| | Block | | | | | | | | | |
| 221 | Method | HATUnf | | | | | | 20% | T3P Method B | |
| | | | | | | | | TFA | | |
| | Building | Acd0559 | | | | | | | | |
| | Block | | | | | | | | | |
| 222 | Method | HATUnf | | | | | | 20% | T3P Method B | |
| | | | | | | | | TFA | | |
| | Building | Acd0505 | | | | | | | | |
| | Block | | | | | | | | | |
| 223 | Method | HATUnf | | | | | | 20% | T3P Method B | |
| | | | | | | | | TFA | | |
| | Building | Acd0503 | | | | | | | | |
| | Block | | | | | | | | | |
| 224 | Method | HATUnf | | | | | | 20% | T3P Method B | |
| | | | | | | | | TFA | | |
| | Building | Acd0532 | | | | | | | | |
| | Block | | | | | | | | | |
| 225 | Method | HATUnf | | | | | | 20% | T3P Method B | |
| | | | | | | | | TFA | | |
| | Building | Acd0503 | | | | | | | | |
| | Block | | | | | | | | | |
| 226\| | Method | HATUnf | | | | | | 20% | T3P Method B | |
| | | | | | | | | TFA | | |
| | Building | Acd0540 | | | | | | | | |
| | Block | | | | | | | | | |
| 227 | Method | HATUnf | | | | | | 20% | T3P Method B | |
| | | | | | | | | TFA | | |
| | Building | Acd0559 | | | | | | | | |
| | Block | | | | | | | | | |
| 228 | \|Method | HATUnf | | | | | | 20% | T3P Method B | |
| | | | | | | | | TFA | | |
| | Building | Acd0505 | | | | | | | | |
| | Block | | | | | | | | | |
| 229 | Method | HATUnf | | | | | | 20% | T3P Method B | |
| | | | | | | | | TFA | | |
| | Building | Acd0317 | | | | | | | | |
| | Block | | | | | | | | | |
| 230 | Method | HATUnf | | | | | | 20% | T3P Method A | |
| | | | | | | | | TFA | | |
| | Building | Acd0505 | | | | | | | | |
| | Block | | | | | | | | | |
| 231 | Method | HATUnf | | | | | | 20% | T3P Method A | |
| | | | | | | | | TFA | | |
| | Building | Acd0503 | | | | | | | | |
| | Block | | | | | | | | | |
| 232 | Method | HATUnf | | | | | | 20% | T3P Method A | |
| | | | | | | | | TFA | | |
| | Building | Acd0540 | | | | | | | | |
| | Block | | | | | | | | | |
| 233 | Method | HATUnf | | | | | | 20% | T3P Method B | |
| | | | | | | | | TFA | | |
| | Building | Acd0505 | | | | | | | | |
| | Block | | | | | | | | | |
| 234 | Method | HATUnf | | | | | | 20% | PyBop Method | |
| | | | | | | | | TFA | B | |
| | Building | Acd0486 | | | | | | | | |
| | Block | | | | | | | | | |
| 235 | Method | HATUnf | | | | | | 20% | T3P Method B | |
| | | | | | | | | TFA | | |
| | Building | Acd0532 | | | | | | | | |
| | Block | | | | | | | | | |
| 236 | Method | HATUnf | | | | | | 20% | T3P Method B | |
| | | | | | | | | TFA | | |
| | Building | Acd0504 | | | | | | | | |
| | Block | | | | | | | | | |
| 237 | Method | HATUnf | | | | | | 20% | T3P Method B | |
| | | | | | | | | TFA | | |
| | Building | Acd0505 | | | | | | | | |
| | Block | | | | | | | | | |
| 238 | Method | HATUnf | | | | | | 20% | T3P Method B | |
| | | | | | | | | TFA | | |
| | Building | Acd0540 | | | | | | | | |
| | Block | | | | | | | | | |
| 239 | Method | HATUnf | | | | | | 20% | PyBop Method | |
| | | | | | | | | TFA | B | |
| | Building | Acd0486 | | | | | | | | |
| | Block | | | | | | | | | |
| 240 | Method | HATUnf | | | | | | 20% | T3P Method A | |
| | | | | | | | | TFA | | |
| | Building | Acd0486 | | | | | | | | |
| | Block | | | | | | | | | |
| 241 | Method | HATUnf | | | | | | 20% | PyBop Method | |
| | | | | | | | | TFA | B | |
| | Building | Acd0486 | | | | | | | | |
| | Block | | | | | | | | | |
| 242 | Method | HATUnf | | | | | | 20% | PyBop Method | |
| | | | | | | | | TFA | B | |
| | Building | Acd0486 | | | | | | | | |
| | Block | | | | | | | | | |
| 243 | Method | HATUnf | | | | | | 20% | T3P Method A | |
| | | | | | | | | TFA | | |
| | Building | Acd0486 | | | | | | | | |
| | Block | | | | | | | | | |
| 244 | Method | HATUnf | | | | | | 20% | PyBop Method | |
| | | | | | | | | TFA | B | |
| | Building | Acd0486 | | | | | | | | |
| | Block | | | | | | | | | |
| 245 | Method | HATUnf | | | | | | 20% | T3P Method A | |
| | | | | | | | | TFA | | |
| | Building | Acd0540 | | | | | | | | |
| | Block | | | | | | | | | |
| 246 | Method | HATUnf | | | | | | 20% | T3P Method A | |
| | | | | | | | | TFA | | |
| | Building | Acd0540 | | | | | | | | |
| | Block | | | | | | | | | |
| 247 | Method | HATUnf | | | | | | 20% | T3P Method A | |
| | | | | | | | | TFA | | |
| | Building | Acd0540 | | | | | | | | |
| | Block | | | | | | | | | |
| 248 | Method | HATUnf | | | | | | 20% | PyBop Method | |
| | | | | | | | | TFA | B | |
| | Building | Acd0486 | | | | | | | | |
| | Block | | | | | | | | | |
| 249 | Method | HATUnf | | | | | | 20% | PyBop Method | |
| | | | | | | | | TFA | B | |
| | Building | Acd0486 | | | | | | | | |
| | Block | | | | | | | | | |
| 250 | Method | HATUnf | | | | | | 20% | T3P Method B | |
| | | | | | | | | TFA | | |
| | Building | Acd0486 | | | | | | | | |
| | Block | | | | | | | | | |
| 251 | Method | HATUnf | | | | | | 20% | PyBop Method | |
| | | | | | | | | TFA | B | |
| | Building | Acd0486 | | | | | | | | |
| | Block | | | | | | | | | |
| 252 | Method | HATUnf | | | | | | 20% | PyBop Method | |
| | | | | | | | | TFA | B | |
| | Building | Acd0486 | | | | | | | | |
| | Block | | | | | | | | | |
| 253 | Method | | | | | | | 20% | T3P Method B | |
| | | | | | | | | TFA | | |
| | Building | | | | | | | | | |
| | Block | | | | | | | | | |
| 254 | Method | | | | | | | 20% | T3P Method B | |
| | | | | | | | | TFA | | |
| | Building | | | | | | | | | |
| | Block | | | | | | | | | |
| 255 | Method | HATUnf | | | | | | 20% | T3P Method A | |
| | | | | | | | | TFA | | |
| | Building | Acd0577 | | | | | | | | |
| | Block | | | | | | | | | |
| 256 | Method | HATUnf | | | | | | 20% | PyBop Method | |
| | | | | | | | | TFA | B | |
| | Building | Acd0578 | | | | | | | | |
| | Block | | | | | | | | | |
| 257 | Method | | | | | | | 20% | T3P Method B | |
| | | | | | | | | TFA | | |
| | Building | | | | | | | | | |
| | Block | | | | | | | | | |
| 258 | \|Method | HATUnf | | | | | | 20% | T3P Method B | |
| | | | | | | | | TFA | | |
| | Building | Acd0505 | | | | | | | | |
| | Block | | | | | | | | | |
| 259 | Method | HATUnf | | | | | | 20% | T3P Method B | |
| | | | | | | | | TFA | | |
| | Building | Acd0532 | | | | | | | | |
| | Block | | | | | | | | | |
| 260 | Method | HATUnf | | | | | | 20% | T3P Method A | |
| | | | | | | | | TFA | | |
| | Building | Acd0317 | | | | | | | | |
| | Block | | | | | | | | | |
| 261 | Method | HATUnf | | | | | | 20% | T3P Method A | |
| | | | | | | | | TFA | | |
| | Building | Acd0317 | | | | | | | | |
| | Block | | | | | | | | | |
| 262 | Method | HATUnf | | | | | | 20% | PyBop Method | |
| | | | | | | | | TFA | B | |
| | Building | Acd0486 | | | | | | | | |
| | Block | | | | | | | | | |
| 263 | Method | HATUnf | | | | | | 20% | PyBop Method | |
| | | | | | | | | TFA | B | |
| | Building | Acd0486 | | | | | | | | |
| | Block | | | | | | | | | |
| 264 | Method | HATUnf | | | | | | 20% | T3P Method A | |
| | | | | | | | | TFA | | |
| | Building | Acd0317 | | | | | | | | |
| | Block | | | | | | | | | |
| 265 | Method | HATUnf | | | | | | 20% | T3P Method B | |
| | | | | | | | | TFA | | |
| | Building | Acd0317 | | | | | | | | |
| | Block | | | | | | | | | |
| 266 | Method | HATUnf | | | | | | 20% | PyBop Method | |
| | | | | | | | | TFA | B | |
| | Building | Acd0486 | | | | | | | | |
| | Block | | | | | | | | | |
| 267 | Method | HATUnf | | | | | | 20% | T3P Method A | |
| | | | | | | | | TFA | | |
| | Building | Acd0401 | | | | | | | | |
| | Block | | | | | | | | | |
| 268 | Method | HATUnf | | | | | | 20% | T3P Method A | |
| | | | | | | | | TFA | | |
| | Building | Acd0317 | | | | | | | | |
| | Block | | | | | | | | | |
| 269 | Method | HATUnf | | | | | | 20% | T3P Method A | |
| | | | | | | | | TFA | | |
| | Building | Acd0317 | | | | | | | | |
| | Block | | | | | | | | | |
| 270 | Method | HATUnf | | | | | | 20% | T3P Method A | |
| | | | | | | | | TFA | | |
| | Building | Acd0317 | | | | | | | | |
| | Block | | | | | | | | | |
| 271 | Method | HATUnf | | | | | | 30% | T3P Method B | |
| | | | | | | | | TFA | | |
| | Building | Acd0317 | | | | | | | | |
| | Block | | | | | | | | | |
| 272 | Method | HATUnf | | | | | | 30% | PyBop Method | |
| | | | | | | | | TFA | B | |
| | Building | Acd0486 | | | | | | | | |
| | Block | | | | | | | | | |
| 273 | Method | HATUnf | | | | | | 20% | PyBop Method | |
| | | | | | | | | TFA | B | |
| | Building | Acd0486 | | | | | | | | |
| | Block | | | | | | | | | |
| 274 | Method | HATUnf | | | | | | 20% | T3P Method A | |
| | | | | | | | | TFA | | |
| | Building | Acd0317 | | | | | | | | |
| | Block | | | | | | | | | |
| 275 | Method | HATUnf | | | | | | 20% | T3P Method A | |
| | | | | | | | | TFA | | |
| | Building | Acd0317 | | | | | | | | |
| | Block | | | | | | | | | |
| 276 | Method | HATUnf | | | | | | 20% | T3P Method B | |
| | | | | | | | | TFA | | |
| | Building | Acd0317 | | | | | | | | |
| | Block | | | | | | | | | |
| 277 | Method | HATUnf | | | | | | 20% | T3P Method A | |
| | | | | | | | | TFA | | |
| | Building | Acd0317 | | | | | | | | |
| | Block | | | | | | | | | |
| 278 | Method | HATUnf | | | | | | 20% | T3P Method B | |
| | | | | | | | | TFA | | |
| | Building | Acd0317 | | | | | | | | |
| | Block | | | | | | | | | |
| 279 | Method | HATUnf | | | | | | 20% | PyBop Method | |
| | | | | | | | | TFA | B | |
| | Building | Acd0486 | | | | | | | | |
| | Block | | | | | | | | | |
| 280 | Method | HATUnf | | | | | | 20% | PyBop Method | |
| | | | | | | | | TFA | B | |
| | Building | Acd0486 | | | | | | | | |
| | Block | | | | | | | | | |
| 281 | Method | HATUnf | | | | | | 20% | PyBop Method | |
| | | | | | | | | TFA | B | |
| | Building | Acd0486 | | | | | | | | |
| | Block | | | | | | | | | |
| 282 | Method | HATUnf | | | | | | 20% | PyBop Method | |
| | | | | | | | | TFA | B | |
| | Building | Acd0486 | | | | | | | | |
| | Block | | | | | | | | | |
| 283 | Method | HATUnf | | | | | | 20% | PyBop Method | |
| | | | | | | | | TFA | B | |
| | Building | Acd0486 | | | | | | | | |
| | Block | | | | | | | | | |
| 284 | Method | HATUnf | | | | | | 20% | PyBop Method | |
| | | | | | | | | TFA | B | |
| | Building | Acd0486 | | | | | | | | |
| | Block | | | | | | | | | |
| 285 | Method | HATUnf | | DdeR | | Morph | | 20% | PyBop Method | |
| | | | | | | | | TFA | B | |
| | Building | Acd0486 | | | | B2BE | | | | |
| | Block | | | | | | | | | |
| 286 | Method | HATUnf | | | | | | 20% | PyBop Method | |
| | | | | | | | | TFA | B | |
| | Building | Acd0486 | | | | | | | | |
| | Block | | | | | | | | | |
| 287 | Method | HATUnf | | | | | | 20% | PyBop Method | |
| | | | | | | | | TFA | B | |
| | Building | Acd0486 | | | | | | | | |
| | Block | | | | | | | | | |
| 288 | Method | HATUnf | | | | | | 20% | PyBop Method | |
| | | | | | | | | TFA | B | |
| | Building | Acd0486 | | | | | | | | |
| | Block | | | | | | | | | |
| 289 | Method | HATUnf | | | | | | 20% | T3P Method B | |
| | | | | | | | | TFA | | |
| | Building | Acd0317 | | | | | | | | |
| | Block | | | | | | | | | |
| 290 | Method | HATUnf | | | | | | 20% | T3P Method B | |
| | | | | | | | | TFA | | |
| | Building | Acd0317 | | | | | | | | |
| | Block | | | | | | | | | |
| 291 | Method | HATUnf | | | | | | 20% | T3P Method B | |
| | | | | | | | | TFA | | |
| | Building | Acd0540 | | | | | | | | |
| | Block | | | | | | | | | |
| 292 | Method | HATUnf | | | | | | 20% | T3P Method B | |
| | | | | | | | | TFA | | |
| | Building | Acd0317 | | | | | | | | |
| | Block | | | | | | | | | |
| 293 | Method | HATUnf | | | | | | 20% | PyBop Method | |
| | | | | | | | | TFA | B | |
| | Building | Acd0486 | | | | | | | | |
| | Block | | | | | | | | | |
| 294 | Method | HATUnf | | | | | | 20% | PyBop Method | |
| | | | | | | | | TFA | B | |
| | Building | Acd0486 | | | | | | | | |
| | Block | | | | | | | | | |
| 295 | Method | HATUnf | | | | | | 20% | PyBop Method | |
| | | | | | | | | TFA | B | |
| | Building | Acd0486 | | | | | | | | |
| | Block | | | | | | | | | |
| 296 | Method | HATUnf | | | | | | 20% | T3P Method A | |
| | | | | | | | | TFA | | |
| | Building | Acd0317 | | | | | | | | |
| | Block | | | | | | | | | |
| 297 | Method | HATUnf | | | | | | 20% | T3P Method A | |
| | | | | | | | | TFA | | |
| | Building | Acd0317 | | | | | | | | |
| | Block | | | | | | | | | |
| 298 | Method | HATUnf | | | | | | 20% | T3P Method B | |
| | | | | | | | | TFA | | |
| | Building | Acd0317 | | | | | | | | |
| | Block | | | | | | | | | |
| 299 | Method | HATUnf | | | | | | 20% | T3P Method A | |
| | | | | | | | | TFA | | |
| | Building | Acd0317 | | | | | | | | |
| | Block | | | | | | | | | |
| 300 | Method | HATUnf | | | | | | 20% | T3P Method A | |
| | | | | | | | | TFA | | |
| | Building | Acd0317 | | | | | | | | |
| | Block | | | | | | | | | |
| 301 | Method | HATUnf | | | | | | 20% | T3P Method A | |
| | | | | | | | | TFA | | |
| | Building | Acd0317 | | | | | | | | |
| | Block | | | | | | | | | |
| 302 | Method | HATUnf | | | | | | 20% | T3P Method A | |
| | | | | | | | | TFA | | |
| | Building | Acd0317 | | | | | | | | |
| | Block | | | | | | | | | |
| 303 | Method | | | | | | | 20% | T3P Method A | |
| | | | | | | | | TFA | | |
| | Building | | | | | | | | | |
| | Block | | | | | | | | | |
| 304 | Method | HATUnf | | | | | | 20% | PyBop Method | |
| | | | | | | | | TFA | B | |
| | Building | Acd0486 | | | | | | | | |
| | Block | | | | | | | | | |
| 305 | Method | HATUnf | | | | | | 20% | T3P Method A | |
| | | | | | | | | TFA | | |
| | Building | Acd0317 | | | | | | | | |
| | Block | | | | | | | | | |
| 306 | Method | HATUnf | | | | | | 20% | T3P Method A | |
| | | | | | | | | TFA | | |
| | Building | Acd0317 | | | | | | | | |
| | Block | | | | | | | | | |
| 307 | Method | HATUnf | | | | | | 20% | T3P Method A | |
| | | | | | | | | TFA | | |
| | Building | Acd0317 | | | | | | | | |
| | Block | | | | | | | | | |
| 308 | Method | HATUnf | | DdeR | | HATU | | 20% | PyBop Method | |
| | | | | | | | | TFA | B | |
| | Building | Acd0486 | | | | RA245 | | | | |
| | Block | | | | | | | | | |
| 309 | Method | HATUnf | | DdeR | | HATU | | 20% | PyBop Method | |
| | | | | | | | | TFA | B | |
| | Building | Acd0486 | | | | RA245 | | | | |
| | Block | | | | | | | | | |
| 310 | Method | HATUnf | | DdeR | | HATU | | 20% | PyBop Method | |
| | | | | | | | | TFA | B | |
| | Building | Acd0486 | | | | RA245 | | | | |
| | Block | | | | | | | | | |
| 311 | Method | HATUnf | | DdeR | | HATU | | 20% | T3P Method A | |
| | | | | | | | | TFA | | |
| | Building | Acd0317 | | | | RA245 | | | | |
| | Block | | | | | | | | | |
| 312 | Method | HATUnf | | DdeR | | HATU | | 20% | T3P Method A | |
| | | | | | | | | TFA | | |
| | Building | Acd0317 | | | | RA245 | | | | |
| | Block | | | | | | | | | |
| 313 | Method | HATUnf | | | | | | 20% | T3P Method B | |
| | | | | | | | | TFA | | |
| | Building | Acd0486 | | | | | | | | |
| | Block | | | | | | | | | |
| 314 | Method | HATUnf | | | | | | 20% | T3P Method B | |
| | | | | | | | | TFA | | |
| | Building | Acd0540 | | | | | | | | |
| | Block | | | | | | | | | |
| 315 | Method | HATUnf | | | | | | 20% | T3P Method B | |
| | | | | | | | | TFA | | |
| | Building | Acd0540 | | | | | | | | |
| | Block | | | | | | | | | |
| 316 | Method | HATUnf | | | | | | 20% | T3P Method B | |
| | | | | | | | | TFA | | |
| | Building | Acd0505 | | | | | | | | |
| | Block | | | | | | | | | |
| 317 | Method | HATUnf | | | | | | 20% | T3P Method B | |
| | | | | | | | | TFA | | |
| | Building | Acd0588 | | | | | | | | |
| | Block | | | | | | | | | |
| 318 | Method | HATUnf | | | | | | 20% | T3P Method B | |
| | | | | | | | | TFA | | |
| | Building | Acd0592 | | | | | | | | |
| | Block | | | | | | | | | |
| 319 | Method | HATUnf | | | | | | 20% | T3P Method A | |
| | | | | | | | | TFA | | |
| | Building | Acd0317 | | | | | | | | |
| | Block | | | | | | | | | |
| 320 | Method | HATUnf | | | | | | 20% | T3P Method A | |
| | | | | | | | | TFA | | |
| | Building | Acd0540 | | | | | | | | |
| | Block | | | | | | | | | |
| 321\| | Method | HATUnf | | | | | | 20% | T3P Method B | |
| | | | | | | | | TFA | | |
| | Building | Acd0540 | | | | | | | | |
| | Block | | | | | | | | | |
| 322 | Method | HATUnf | | | | | | 20% | PyBop Method | |
| | | | | | | | | TFA | B | |
| | Building | Acd0486 | | | | | | | | |
| | Block | | | | | | | | | |
| 323 | Method | HATUnf | | | | | | 20% | PyBop Method | |
| | | | | | | | | TFA | B | |
| | Building | Acd0486 | | | | | | | | |
| | Block | | | | | | | | | |
| 324 | Method | HATUnf | | | | | | 20% | PyBop Method | |
| | | | | | | | | TFA | B | |
| | Building | Acd0486 | | | | | | | | |
| | Block | | | | | | | | | |
| 325 | Method | HATUnf | | | | | | 20% | T3P Method A | |
| | | | | | | | | TFA | | |
| | Building | Acd0317 | | | | | | | | |
| | Block | | | | | | | | | |
| 326 | Method | HATUnf | | | | | | 20% | T3P Method B | |
| | | | | | | | | TFA | | |
| | Building | Acd0540 | | | | | | | | |
| | Block | | | | | | | | | |
| 327 | Method | HATUnf | | | | | | 20% | T3P Method A | |
| | | | | | | | | TFA | | |
| | Building | Acd0317 | | | | | | | | |
| | Block | | | | | | | | | |
| 328 | Method | HATUnf | | | | | | 20% | T3P Method B | |
| | | | | | | | | TFA | | |
| | Building | Acd0317 | | | | | | | | |
| | Block | | | | | | | | | |
| 329 | Method | HATUnf | | | | | | \|20% | T3P Method B | |
| | | | | | | | | TFA | | |
| | Building | Acd0588 | | | | | | | | |
| | Block | | | | | | | | | |
| 330 | Method | HATUnf | | | | | | 20% | T3P Method B | |
| | | | | | | | | TFA | | |
| | Building | Acd0588 | | | | | | | | |
| | Block | | | | | | | | | |
| 331 | Method | HATUnf | | | | | | 20% | T3P Method B | |
| | | | | | | | | TFA | | |
| | Building | Acd0505 | | | | | | | | |
| | Block | | | | | | | | | |
| 332 | Method | HATUnf | | | | | | 20% | T3P Method B | |
| | | | | | | | | TFA | | |
| | Building | Acd0486 | | | | | | | | |
| | Block | | | | | | | | | |
| 333 | Method | HATUnf | | | | | | 20% | PyBop Method | |
| | | | | | | | | TFA | B | |
| | Building | Acd0486 | | | | | | | | |
| | Block | | | | | | | | | |
| 334 | Method | HATUnf | | | | | | 20% | PyBop Method | |
| | | | | | | | | TFA | B | |
| | Building | Acd0486 | | | | | | | | |
| | Block | | | | | | | | | |
| 335 | Method | HATUnf | | | | | | 20% | PyBop Method | |
| | | | | | | | | TFA | B | |
| | Building | Acd0486 | | | | | | | | |
| | Block | | | | | | | | | |
| 336 | Method | HATUnf | | | | | | 20% | PyBop Method | |
| | | | | | | | | TFA | B | |
| | Building | Acd0486 | | | | | | | | |
| | Block | | | | | | | | | |
| 337 | Method | HATUnf | | | | | | 20% | PyBop Method | |
| | | | | | | | | TFA | B | |
| | Building | Acd0486 | | | | | | | | |
| | Block | | | | | | | | | |
| 338 | Method | HATUnf | | | | | | 20% | PyBop Method | |
| | | | | | | | | TFA | B | |
| | Building | Acd0486 | | | | | | | | |
| | Block | | | | | | | | | |
| 339 | Method | HATUnf | | | | | | 20% | T3P Method B | |
| | | | | | | | | TFA | | |
| | Building | Acd0317 | | | | | | | | |
| | Block | | | | | | | | | |
| 340 | Method | HATUnf | | | | | | 20% | T3P Method B | |
| | | | | | | | | TFA | | |
| | Building | Acd0317 | | | | | | | | |
| | Block | | | | | | | | | |
| 341 | Method | HATUnf | | | | | | 20% | T3P Method B | |
| | | | | | | | | TFA | | |
| | Building | Acd0317 | | | | | | | | |
| | Block | | | | | | | | | |
| 342 | Method | HATUnf | | | | | | 20% | T3P Method A | |
| | | | | | | | | TFA | | |
| | Building | Acd0588 | | | | | | | | |
| | Block | | | | | | | | | |
| 343 | Method | HATUnf | | | | | | 20% | T3P Method B | |
| | | | | | | | | TFA | | |
| | Building | Acd0505 | | | | | | | | |
| | Block | | | | | | | | | |
| 344 | Method | HATUnf | | | | | | 20% | T3P Method A | |
| | | | | | | | | TFA | | |
| | Building | Acd0588 | | | | | | | | |
| | Block | | | | | | | | | |
| 345 | Method | HATUnf | | | | | | 20% | T3P Method B | |
| | | | | | | | | TFA | | |
| | Building | Acd0317 | | | | | | | | |
| | Block | | | | | | | | | |
| 346 | Method | HATUnf | | | | | | 20% | T3P Method B | |
| | | | | | | | | TFA | | |
| | Building | Acd0317 | | | | | | | | |
| | Block | | | | | | | | | |
| 347 | Method | HATUnf | | | | | | 20% | T3P Method B | |
| | | | | | | | | TFA | | |
| | Building | Acd0317 | | | | | | | | |
| | Block | | | | | | | | | |
| 348 | Method | HATUnf | | | | | | 20% | PyBop Method | |
| | | | | | | | | TFA | B | |
| | Building | Acd0486 | | | | | | | | |
| | Block | | | | | | | | | |
| 349 | Method | HATUnf | | | | | | 20% | T3P Method B | |
| | | | | | | | | TFA | | |
| | Building | Acd0317 | | | | | | | | |
| | Block | | | | | | | | | |
| 350 | Method | HATUnf | | | | | | 20% | T3P Method B | |
| | | | | | | | | TFA | | |
| | Building | Acd0559 | | | | | | | | |
| | Block | | | | | | | | | |
| 351 | Method | HATUnf | | | | | | 20% | PyBop Method | |
| | | | | | | | | TFA | B | |
| | Building | Acd0486 | | | | | | | | |
| | Block | | | | | | | | | |
| 352 | Method | HATUnf | | | | | | 20% | T3P Method B | |
| | | | | | | | | TFA | | |
| | Building | Acd0559 | | | | | | | | |
| | Block | | | | | | | | | |
| 353 | Method | HATUnf | | | | | | 20% | T3P Method B | |
| | | | | | | | | TFA | | |
| | Building | Acd0594 | | | | | | | | |
| | Block | | | | | | | | | |
| 354 | Method | HATUnf | | DdeR | | | | 24% | T3P Method A | |
| | | | | | | | | HFIP | | |
| | Building | Acd0486 | | | | | | | | |
| | Block | | | | | | | | | |
| 355 | Method | HATUnf | | DdeR | | | | 20% | PyBop Method | |
| | | | | | | | | TFA | B | |
| | Building | Acd0486 | | | | | | | | |
| | Block | | | | | | | | | |
| 356 | Method | HATUnf | | | | | | 20% | T3P Method B | |
| | | | | | | | | TFA | | |
| | Building | Acd0317 | | | | | | | | |
| | Block | | | | | | | | | |
| 357 | Method | HATUnf | | | | | | 20% | T3P Method B | |
| | | | | | | | | TFA | | |
| | Building | Acd0317 | | | | | | | | |
| | Block | | | | | | | | | |
| 358 | Method | HATUnf | | | | | | 20% | PyBop Method | |
| | | | | | | | | TFA | B | |
| | Building | Acd0486 | | | | | | | | |
| | Block | | | | | | | | | |
| 359 | Method | HATUnf | | | | | | 20% | PyBop Method | |
| | | | | | | | | TFA | B | |
| | Building | Acd0486 | | | | | | | | |
| | Block | | | | | | | | | |
| 360 | Method | HATUnf | | | | | | 20% | PyBop Method | |
| | | | | | | | | TFA | B | |
| | Building | Acd0486 | | | | | | | | |
| | Block | | | | | | | | | |
| 361 | Method | HATUnf | | | | | | 20% | PyBop Method | |
| | | | | | | | | TFA | B | |
| | Building | Acd0486 | | | | | | | | |
| | Block | | | | | | | | | |
| 362 | Method | HATUnf | | | | | | 20% | PyBop Method | |
| | | | | | | | | TFA | B | |
| | Building | Acd0486 | | | | | | | | |
| | Block | | | | | | | | | |
| 363 | Method | HATUnf | | | | | | 20% | T3P Method A | |
| | | | | | | | | TFA | | |
| | Building | Acd0317 | | | | | | | | |
| | Block | | | | | | | | | |
| 364 | Method | HATUnf | | | | | | 20% | PyBop Method | |
| | | | | | | | | TFA | B | |
| | Building | Acd0486 | | | | | | | | |
| | Block | | | | | | | | | |
| 365 | Method | HATUnf | | | | | | 20% | PyBop Method | |
| | | | | | | | | TFA | B | |
| | Building | Acd0486 | | | | | | | | |
| | Block | | | | | | | | | |
| 366 | Method | HATUnf | | | | | | 20% | T3P Method B | |
| | | | | | | | | TFA | | |
| | Building | Acd0486 | | | | | | | | |
| | Block | | | | | | | | | |
| 367 | Method | HATUnf | | | | | | 20% | T3P Method B | |
| | | | | | | | | TFA | | |
| | Building | Acd0596 | | | | | | | | |
| | Block | | | | | | | | | |
| 368 | Method | HATUnf | | | | | | 20% | T3P Method B | |
| | | | | | | | | TFA | | |
| | Building | Acd0596 | | | | | | | | |
| | Block | | | | | | | | | |
| 369 | Method | HATUnf | | | | | | 20% | T3P Method B | |
| | | | | | | | | TFA | | |
| | Building | Acd0596 | | | | | | | | |
| | Block | | | | | | | | | |
| 370 | Method | HATUnf | | | | | | 20% | T3P Method B | |
| | | | | | | | | TFA | | |
| | Building | Acd0596 | | | | | | | | |
| | Block | | | | | | | | | |
| 371 | Method | HATUnf | | | | | | 20% | T3P Method A | |
| | | | | | | | | TFA | | |
| | Building | Acd0317 | | | | | | | | |
| | Block | | | | | | | | | |
| 372 | Method | HATUnf | | | | | | 20% | PyBop Method | |
| | | | | | | | | TFA | B | |
| | Building | Acd0486 | | | | | | | | |
| | Block | | | | | | | | | |
| 373 | Method | HATUnf | | | | | | 20% | PyBop Method | |
| | | | | | | | | TFA | B | |
| | Building | Acd0486 | | | | | | | | |
| | Block | | | | | | | | | |
| 374 | Method | HATUnf | | DdeR | | HATUnf | | 20% | PyBop Method | |
| | | | | | | | | TFA | B | |
| | Building | Acd0486 | | | | RA245 | | | | |
| | Block | | | | | | | | | |
| 375 | Method | HATUnf | | DdeR | | HATUnf | | 20% | PyBop Method | |
| | | | | | | | | TFA | B | |
| | Building | Acd0486 | | | | RA245 | | | | |
| | Block | | | | | | | | | |
| 376 | Method | HATUnf | | DdeR | | HATUnf | | 20% | PyBop Method | |
| | | | | | | | | TFA | B | |
| | Building | Acd0486 | | | | Acd0347 | | | | |
| | Block | | | | | | | | | |
| 377 | Method | HATUnf | | DdeR | | HATUnf | | 20% | PyBop Method | |
| | | | | | | | | TFA | B | |
| | Building | Acd0486 | | | | Acd0445 | | | | |
| | Block | | | | | | | | | |
| 378 | Method | HATUnf | | DdeR | | HATUnf | | 20% | T3P Method A | |
| | | | | | | | | TFA | | |
| | Building | Acd0317 | | | | Acd0347 | | | | |
| | Block | | | | | | | | | |
| 379 | Method | HATUnf | | | | | | 20% | T3P Method B | |
| | | | | | | | | TFA | | |
| | Building | Acd0596 | | | | | | | | |
| | Block | | | | | | | | | |
| 380 | Method | HATUnf | | | | | | 20% | PyBop Method | |
| | | | | | | | | TFA | B | |
| | Building | Acd0486 | | | | | | | | |
| | Block | | | | | | | | | |
| 381 | Method | HATUnf | | | | | | 20% | T3P Method A | |
| | | | | | | | | TFA | | |
| | Building | Acd0588 | | | | | | | | |
| | Block | | | | | | | | | |
| 382 | Method | HATUnf | | | | | | 20% | T3P Method B | |
| | | | | | | | | TFA | | |
| | Building | Acd0317 | | | | | | | | |
| | Block | | | | | | | | | |
| 383 | Method | HATUnf | | DdeR | | HATUnf | | 20% | T3P Method A | |
| | | | | | | | | TFA | | |
| | Building | Acd0317 | | | | RA245 | | | | |
| | Block | | | | | | | | | |
| 384 | Method | HATUnf | | DdeR | | HATUnf | | 20% | T3P Method A | |
| | | | | | | | | TFA | | |
| | Building | Acd0317 | | | | RA245 | | | | |
| | Block | | | | | | | | | |
| 385 | Method | HATUnf | | DdeR | | HATUnf | | \|20% | T3P Method A | |
| | | | | | | | | TFA | | |
| | Building | Acd0505 | | | | RA245 | | | | |
| | Block | | | | | | | | | |
| 386 | Method | HATUnf | | DdeR | | HATUnf | | 20% | T3P Method A | |
| | | | | | | | | TFA | | |
| | Building | Acd0317 | | | | RA245 | | | | |
| | Block | | | | | | | | | |
| 387 | Method | HATUnf | | DdeR | | HATUnf | | 20% | T3P Method A | |
| | | | | | | | | TFA | | |
| | Building | Acd0317 | | | | RA245 | | | | |
| | Block | | | | | | | | | |
| 388 | Method | HATUnf | | DdeR | | HATUnf | | 20% | T3P Method A | |
| | | | | | | | | TFA | | |
| | Building | Acd0317 | | | | RA245 | | | | |
| | Block | | | | | | | | | |
| 389 | Method | HATUnf | | | | | | 20% | PyBop Method | |
| | | | | | | | | TFA | B | |
| | Building | Acd0486 | | | | | | | | |
| | Block | | | | | | | | | |
| 390 | Method | HATUnf | | | | | | 20% | PyBop Method | |
| | | | | | | | | TFA | B | |
| | Building | Acd0486 | | | | | | | | |
| | Block | | | | | | | | | |
| 391 | Method | HATUnf | | | | | | 20% | PyBop Method | |
| | | | | | | | | TFA | B | |
| | Building | Acd0486 | | | | | | | | |
| | Block | | | | | | | | | |
| 392 | Method | HATUnf | | | | | | 20% | T3P Method A | |
| | | | | | | | | TFA | | |
| | Building | Acd0317 | | | | | | | | |
| | Block | | | | | | | | | |
| 393 | Method | HATUnf | | | | | | 20% | T3P Method A | |
| | | | | | | | | TFA | | |
| | Building | Acd0317 | | | | | | | | |
| | Block | | | | | | | | | |
| 394 | Method | HATUnf | | | | | | 20% | PyBop Method | |
| | | | | | | | | TFA | B | |
| | Building | Acd0486 | | | | | | | | |
| | Block | | | | | | | | | |
| 395 | Method | HATUnf | | | | | | 20% | PyBop Method | |
| | | | | | | | | TFA | B | |
| | Building | Acd0486 | | | | | | | | |
| | Block | | | | | | | | | |
| 396 | Method | HATUnf | | | | | | 20% | T3P Method A | |
| | | | | | | | | TFA | | |
| | Building | Acd0540 | | | | | | | | |
| | Block | | | | | | | | | |
| 397 | Method | HATUnf | | DdeR | | Morph | | 20% | T3P Method A | |
| | | | | | | | | TFA | | |
| | Building | Acd0540 | | | | B2BE | | | | |
| | Block | | | | | | | | | |
| 398 | Method | HATUnf | | DdeR | | HATUnf | | 20% | PyBop Method | |
| | | | | | | | | TFA | B | |
| | Building | Acd0486 | | | | RA245 | | | | |
| | Block | | | | | | | | | |
| 399 | Method | HATUnf | | DdeR | | HATUnf | | 20% | PyBop Method | |
| | | | | | | | | TFA | B | |
| | Building | Acd0486 | | | | RA211 | | | | |
| | Block | | | | | | | | | |
| 400 | Method | HATUnf | | DdeR | | | | 24% | T3P Method B | |
| | | | | | | | | HFIP | | |
| | Building | Acd0486 | | | | | | | | |
| | Block | | | | | | | | | |
| 401 | Method | HATUnf | | | | | | 20% | T3P Method B | |
| | | | | | | | | TFA | | |
| | Building | Acd0486 | | | | | | | | |
| | Block | | | | | | | | | |
| 402 | Method | HATUnf | | DdeR | | | | 24% | T3P Method B | |
| | | | | | | | | HFIP | | |
| | Building | Acd0588 | | | | | | | | |
| | Block | | | | | | | | | |
| 403 | Method | HATUnf | | | | | | 20% | T3P Method A | |
| | | | | | | | | TFA | | |
| | Building | Acd0588 | | | | | | | | |
| | Block | | | | | | | | | |
| 404 | Method | HATUnf | | | | | | 20% | T3P Method B | |
| | | | | | | | | TFA | | |
| | Building | Acd0487 | | | | | | | | |
| | Block | | | | | | | | | |
| 405 | Method | HATUnf | | DdeR | | | | 24% | T3P Method B | Boc |
| | | | | | | | | HFIP | | |
| | Building | Acd0625 | | | | | | | | |
| | Block | | | | | | | | | |
| 406 | Method | HATUnf | | DdeR | | | | 24% | T3P Method B | Boc |
| | | | | | | | | HFIP | | |
| | Building | Acd0626 | | | | | | | | |
| | Block | | | | | | | | | |
| 407 | Method | HATUnf | | | | | | 20% | T3P Method A | |
| | | | | | | | | TFA | | |
| | Building | Acd0317 | | | | | | | | |
| | Block | | | | | | | | | |
| 408 | Method | HATUnf | | DdeR | | | | 20% | PyBop Method | |
| | | | | | | | | TFA | B | |
| | Building | Acd0486 | | | | | | | | |
| | Block | | | | | | | | | |
| 409 | Method | HATUnf | | | | | | 20% | T3P Method A | |
| | | | | | | | | TFA | | |
| | Building | Acd0486 | | | | | | | | |
| | Block | | | | | | | | | |
| 410 | Method | HATUnf | | | | | | 20% | T3P Method A | |
| | | | | | | | | TFA | | |
| | Building | Acd0486 | | | | | | | | |
| | Block | | | | | | | | | |
| 411 | Method | HATUnf | | | | | | 20% | T3P Method B | |
| | | | | | | | | TFA | | |
| | Building | Acd0486 | | | | | | | | |
| | Block | | | | | | | | | |
| 412 | Method | HATUnf | | DdeR | | | | 20% | T3P Method B | |
| | | | | | | | | TFA | | |
| | Building | Acd0486 | | | | | | | | |
| | Block | | | | | | | | | |
| 413 | Method | HATUnf | | DdeR | | | | 20% | T3P Method B | |
| | | | | | | | | TFA | | |
| | Building | Acd0486 | | | | | | | | |
| | Block | | | | | | | | | |
| 414 | Method | HATUnf | | | | | | 20% | T3P Method B | |
| | | | | | | | | TFA | | |
| | Building | Acd0540 | | | | | | | | |
| | Block | | | | | | | | | |
| 415 | Method | HATUnf | | | | | | 20% | T3P Method B | |
| | | | | | | | | TFA | | |
| | Building | Acd0540 | | | | | | | | |
| | Block | | | | | | | | | |
| 416 | Method | HATUnf | | DdeR | | | | 24% | T3P Method B | |
| | | | | | | | | HFIP | | |
| | Building | Acd0486 | | | | | | | | |
| | Block | | | | | | | | | |
| 417 | Method | HATUnf | | DdeR | | | | 24% | T3P Method B | |
| | | | | | | | | HFIP | | |
| | Building | Acd0486 | | | | | | | | |
| | Block | | | | | | | | | |
| 418 | Method | HATUnf | | DdeR | | | | 24% | T3P Method B | |
| | | | | | | | | HFIP | | |
| | Building | Acd0486 | | | | | | | | |
| | Block | | | | | | | | | |
| 419 | Method | HATUnf | | | | | | 20% | T3P Method B | |
| | | | | | | | | TFA | | |
| | Building | Acd0540 | | | | | | | | |
| | Block | | | | | | | | | |
| 420 | Method | HATUnf | | | | | | 20% | T3P Method B | |
| | | | | | | | | TFA | | |
| | Building | Acd0540 | | | | | | | | |
| | Block | | | | | | | | | |
| 421 | Method | HATUnf | | DdeR | | | | 24% | T3P Method B | |
| | | | | | | | | HFIP | | |
| | Building | Acd0540 | | | | | | | | |
| | Block | | | | | | | | | |
| 422 | Method | HATUnf | | | | | | 20% | PyBop Method | |
| | | | | | | | | TFA | B | |
| | Building | Acd0486 | | | | | | | | |
| | Block | | | | | | | | | |
| 423 | Method | HATUnf | | DdeR | | | | 24% | T3P Method A | Boc |
| | | | | | | | | HFIP | | |
| | Building | Acd0625 | | | | | | | | |
| | Block | | | | | | | | | |
| 424 | Method | HATUnf | | | | | | 20% | T3P Method B | |
| | | | | | | | | TFA | | |
| | Building | Acd0486 | | | | | | | | |
| | Block | | | | | | | | | |
| 425 | Method | HATUnf | | DdeR | | | | 24% | T3P Method B | Boc |
| | | | | | | | | HFIP | | |
| | Building | Acd0625 | | | | | | | | |
| | Block | | | | | | | | | |
| 426 | Method | HATUnf | | | | | | 20% | T3P Method B | |
| | | | | | | | | TFA | | |
| | Building | Acd0588 | | | | | | | | |
| | Block | | | | | | | | | |
| 427 | Method | HATUnf | | | | | | 20% | PyBop Method | |
| | | | | | | | | TFA | B | |
| | Building | Acd0687 | | | | | | | | |
| | Block | | | | | | | | | |
| 428 | Method | HATUnf | | | | | | 20% | PyBop Method | |
| | | | | | | | | TFA | B | |
| | Building | Acd0688 | | | | | | | | |
| | Block | | | | | | | | | |
| 429 | Method | KO | | | | | | 20% | T3P Method B | |
| | | | | | | | | TFA | | |
| | Building | Acd0525 | | | | | | | | |
| | Block | | | | | | | | | |
| 430 | Method | HATUnf | | | | | | 20% | T3P Method B | |
| | | | | | | | | TFA | | |
| | Building | Acd0486 | | | | | | | | |
| | Block | | | | | | | | | |
| 431 | Method | | | | | | | 20% | T3P Method B | |
| | | | | | | | | TFA | | |
| | Building | | | | | | | | | |
| | Block | | | | | | | | | |
| 432 | Method | | | DdeR | | Morph | | 20% | T3P Method B | |
| | | | | | | | | TFA | | |
| | Building | | | | | B2BE | | | | |
| | Block | | | | | | | | | |
| 433 | Method | | | DdeR | | Morph | | 20% | T3P Method B | |
| | | | | | | | | TFA | | |
| | Building | | | | | B2BE | | | | |
| | Block | | | | | | | | | |
| 434 | Method | | | DdeR | | Morph | | 20% | T3P Method B | |
| | | | | | | | | TFA | | |
| | Building | | | | | B2BE | | | | |
| | Block | | | | | | | | | |
| 435 | Method | | | DdeR | | Morph | | 20% | T3P Method B | |
| | | | | | | | | TFA | | |
| | Building | | | | | B2BE | | | | |
| | Block | | | | | | | | | |
| 436 | Method | | | | | | | 20% | T3P Method A | |
| | | | | | | | | TFA | | |
| | Building | | | | | | | | | |
| | Block | | | | | | | | | |
| 437 | Method | HATUnf | | | | | | 20% | T3P Method B | |
| | | | | | | | | TFA | | |
| | Building | Acd0532 | | | | | | | | |
| | Block | | | | | | | | | |
| 438 | Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 439 | Method | HATUnf | | DdeR | | | | 24% | PyBop Method | |
| | | | | | | | | HFIP | B | |
| | Building | Acd0486 | | | | | | | | |
| | Block | | | | | | | | | |
| 440 | Method | HATUnf | | DdeR | | | | 24% | PyBop Method | |
| | | | | | | | | HFIP | B | |
| | Building | Acd0486 | | | | | | | | |
| | Block | | | | | | | | | |
| 441 | Method | HATUnf | | | | | | 24% | T3P Method B | |
| | | | | | | | | HFIP | | |
| | Building | Acd0486 | | | | | | | | |
| | Block | | | | | | | | | |
| 442 | Method | | | | | | | 20% | T3P Method B | |
| | | | | | | | | TFA | | |
| | Building | | | | | | | | | |
| | Block | | | | | | | | | |
| 443 | Method | HATUnf | | DdeR | | | | 24% | T3P Method B | |
| | | | | | | | | HFIP | | |
| | Building | Acd0486 | | | | | | | | |
| | Block | | | | | | | | | |
| 444 | Method | HATUnf | | DdeR | | | | 24% | T3P Method B | |
| | | | | | | | | HFIP | | |
| | Building | Acd0486 | | | | | | | | |
| | Block | | | | | | | | | |
| 445 | Method | | | | | | | 20% | T3P Method B | |
| | | | | | | | | TFA | | |
| | Building | | | | | | | | | |
| | Block | | | | | | | | | |
| 446 | Method | | | | | | | 20% | T3P Method B | |
| | | | | | | | | TFA | | |
| | Building | | | | | | | | | |
| | Block | | | | | | | | | |
| 447 | Method | | | | | | | 20% | T3P Method B | |
| | | | | | | | | TFA | | |
| | Building | | | | | | | | | |
| | Block | | | | | | | | | |
| 448 | Method | HATUnf | | | | | | 20% | T3P Method A | |
| | | | | | | | | TFA | | |
| | Building | Acd0317 | | | | | | | | |
| | Block | | | | | | | | | |
| 449 | Method | HATUnf | | | | | | 20% | T3P Method A | |
| | | | | | | | | TFA | | |
| | Building | Acd0317 | | | | | | | | |
| | Block | | | | | | | | | |
| 450 | Method | HATUnf | | | | | | 20% | T3P Method A | |
| | | | | | | | | TFA | | |
| | Building | Acd0317 | | | | | | | | |
| | Block | | | | | | | | | |
| 451 | Method | HATUnf | | | | | | 20% | T3P Method A | |
| | | | | | | | | TFA | | |
| | Building | Acd0486 | | | | | | | | |
| | Block | | | | | | | | | |
| 452 | Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 453 | Method | HATUnf | | | | | | 20% | PyBop Method | |
| | | | | | | | | TFA | B | |
| | Building | Acd0486 | | | | | | | | |
| | Block | | | | | | | | | |
| 454 | Method | HATUnf | | | | | | 20% | T3P Method A | |
| | | | | | | | | TFA | | |
| | Building | Acd0317 | | | | | | | | |
| | Block | | | | | | | | | |
| 455 | Method | HATUnf | | DdeR | | | | 24% | T3P Method B | |
| | | | | | | | | HFIP | | |
| | Building | Acd0486 | | | | | | | | |
| | Block | | | | | | | | | |
| 456 | Method | HATUnf | | DdeR | | | | 24% | PyBop Method | |
| | | | | | | | | HFIP | B | |
| | Building | Acd0486 | | | | | | | | |
| | Block | | | | | | | | | |
| 457 | Method | HATUnf | | | | | | 20% | T3P Method B | |
| | | | | | | | | TFA | | |
| | Building | Acd0588 | | | | | | | | |
| | Block | | | | | | | | | |
| 458 | Method | HATUnf | | | | | | 20% | T3P Method B | |
| | | | | | | | | TFA | | |
| | Building | Acd0588 | | | | | | | | |
| | Block | | | | | | | | | |
| 459 | Method | HATUnf | | | | | | 20% | PyBop Method | |
| | | | | | | | | TFA | B | |
| | Building | Acd0486 | | | | | | | | |
| | Block | | | | | | | | | |
| 460 | Method | HATUnf | | | | | | 20% | T3P Method A | |
| | | | | | | | | TFA | | |
| | Building | Acd0588 | | | | | | | | |
| | Block | | | | | | | | | |
| 461 | Method | HATU | Ac | | | | | 20% | T3P Method B | |
| | | | | | | | | TFA | | |
| | Building | Acpc | AcAh | | | | | | | |
| | Block | | | | | | | | | |
| 462 | Method | HATU | HATUnf | | | | | 20% | PyBop Method | |
| | | | | | | | | TFA | B | |
| | Building | Acpc | Acd | | | | | | | |
| | Block | | 0486 | | | | | | | |
| 463 | Method | HATUnf | | DdeR | | | | 24% | T3P Method B | |
| | | | | | | | | HFIP | | |
| | Building | Acd0317 | | | | | | | | |
| | Block | | | | | | | | | |
| 464 | Method | HATUnf | | | | | | 20% | PyBop Method | |
| | | | | | | | | TFA | B | |
| | Building | Acd0486 | | | | | | | | |
| | Block | | | | | | | | | |
| 465 | Method | HATUnf | | | | | | 20% | T3P Method B | |
| | | | | | | | | TFA | | |
| | Building | Acd0588 | | | | | | | | |
| | Block | | | | | | | | | |
| 466 | Method | | | | | | | 20% | T3P Method B | |
| | | | | | | | | TFA | | |
| | Building | | | | | | | | | |
| | Block | | | | | | | | | |
| 467 | Method | HATUnf | | | | | | 20% | T3P Method B | |
| | | | | | | | | TFA | | |
| | Building | Acd0486 | | | | | | | | |
| | Block | | | | | | | | | |
| 468 | Method | HATUnf | | DdeR | MITS | | | 24% | PyBop Method | |
| | | | | | | | | HFIP | B | |
| | Building | Acd0486 | | | MeOH | | | | | |
| | Block | | | | | | | | | |
| 469 | Method | HATUnf | | | | | | 20% | T3P Method A | |
| | | | | | | | | TFA | | |
| | Building | Acd0505 | | | | | | | | |
| | Block | | | | | | | | | |
| 470 | Method | HATUnf | | DdeR | | | | 24% | T3P Method B | |
| | | | | | | | | HFIP | | |
| | Building | Acd0486 | | | | | | | | |
| | Block | | | | | | | | | |
| 471 | Method | HATUnf | | DdeR | | | | 24% | T3P Method B | |
| | | | | | | | | HFIP | | |
| | Building | Acd0486 | | | | | | | | |
| | Block | | | | | | | | | |
| 472 | Method | HATUnf | | | | | | 20% | PyBop Method | |
| | | | | | | | | TFA | B | |
| | Building | Acd0486 | | | | | | | | |
| | Block | | | | | | | | | |
| 473 | Method | HATUnf | | | | | | 20% | PyBop Method | |
| | | | | | | | | TFA | B | |
| | Building | Acd0486 | | | | | | | | |
| | Block | | | | | | | | | |
| 474 | Method | HATUnf | | | | | | 20% | T3P Method B | |
| | | | | | | | | TFA | | |
| | Building | Acd0734 | | | | | | | | |
| | Block | | | | | | | | | |
| 475 | Method | HATUnf | | DdeR | | | | 24% | T3P Method B | |
| | | | | | | | | HFIP | | |
| | Building | Acd0737 | | | | | | | | |
| | Block | | | | | | | | | |
| 476 | Method | HATUnf | | DdeR | | | | 24% | PyBop Method | |
| | | | | | | | | HFIP | B | |
| | Building | Acd0737 | | | | | | | | |
| | Block | | | | | | | | | |
| 477 | Method | HATUnf | | DdeR | | | | 24% | PyBop Method | |
| | | | | | | | | HFIP | B | |
| | Building | Acd0737 | | | | | | | | |
| | Block | | | | | | | | | |
| 478 | Method | HATUnf | | | | | | 20% | PyBop Method | |
| | | | | | | | | TFA | B | |
| | Building | Acd0486 | | | | | | | | |
| | Block | | | | | | | | | |
| 479 | Method | HATUnf | | | | | | 20% | T3P Method B | |
| | | | | | | | | TFA | | |
| | Building | Acd0588 | | | | | | | | |
| | Block | | | | | | | | | |
| 480 | Method | | | | | | | 20% | T3P Method B | |
| | | | | | | | | TFA | | |
| | Building | | | | | | | | | |
| | Block | | | | | | | | | |
| 481 | Method | HATUnf | | | | | | 20% | T3P Method B | |
| | | | | | | | | TFA | | |
| | Building | Acd0747 | | | | | | | | |
| | Block | | | | | | | | | |
| 482 | Method | HATUnf | | DdeR | | | | 24% | T3P Method B | |
| | | | | | | | | HFIP | | |
| | Building | Acd0486 | | | | | | | | |
| | Block | | | | | | | | | |
| 483 | Method | HATUnf | | | | | | 20% | PyBop Method | |
| | | | | | | | | TFA | B | |
| | Building | Acd0486 | | | | | | | | |
| | Block | | | | | | | | | |
| 484 | Method | HATUnf | | | | | | 20% | T3P Method B | |
| | | | | | | | | TFA | | |
| | Building | Acd0588 | | | | | | | | |
| | Block | | | | | | | | | |
| 485 | Method | HATUnf | | | | | | 20% | T3P Method A | |
| | | | | | | | | TFA | | |
| | Building | Acd0588 | | | | | | | | |
| | Block | | | | | | | | | |
| 486 | Method | HATUnf | | | | | | 20% | T3P Method B | |
| | | | | | | | | TFA | | |
| | Building | Acd0588 | | | | | | | | |
| | Block | | | | | | | | | |
| 487 | Method | HATUnf | | DdeR | | | | 24% | T3P Method B | |
| | | | | | | | | HFIP | | |
| | Building | Acd0588 | | | | | | | | |
| | Block | | | | | | | | | |
| 488 | Method | HATUnf | | DdeR | | | | 24% | T3P Method B | tBu |
| | | | | | | | | HFIP | | |
| | Building | Acd0588 | | | | | | | | |
| | Block | | | | | | | | | |
| 489 | Method | HATUnf | | | | | | 20% | T3P Method B | |
| | | | | | | | | TFA | | |
| | Building | Acd0588 | | | | | | | | |
| | Block | | | | | | | | | |
| 490 | Method | HATUnf | | DdeR | | | | 24% | T3P Method B | |
| | | | | | | | | HFIP | | |
| | Building | Acd0588 | | | | | | | | |
| | Block | | | | | | | | | |
| 491 | Method | HATUnf | | | | | | 20% | PyBop Method | |
| | | | | | | | | TFA | B | |
| | Building | Acd0486 | | | | | | | | |
| | Block | | | | | | | | | |
| 492 | Method | HATUnf | | | | | | \|20% | T3P Method A | |
| | | | | | | | | TFA | | |
| | Building | Acd0317 | | | | | | | | |
| | Block | | | | | | | | | |
| 493 | Method | HATUnf | | | | | | 20% | PyBop Method | |
| | | | | | | | | TFA | B | |
| | Building | Acd0486 | | | | | | | | |
| | Block | | | | | | | | | |
| 494 | Method | HATUnf | | DdeR | | | | 24% | PyBop Method | |
| | | | | | | | | HFIP | B | |
| | Building | Acd0486 | | | | | | | | |
| | Block | | | | | | | | | |
| 495 | Method | HATUnf | | DdeR | | | | 24% | PyBop Method | |
| | | | | | | | | HFIP | B | |
| | Building | Acd0486 | | | | | | | | |
| | Block | | | | | | | | | |
| 496 | Method | HATUnf | | DdeR | MITS | | | 24% | PyBop Method | |
| | | | | | | | | HFIP | B | |
| | Building | Acd0486 | | | MeOH | | | | | |
| | Block | | | | | | | | | |
| 497 | Method | HATUnf | | | | | | 20% | T3P Method A | |
| | | | | | | | | TFA | | |
| | Building | Acd0588 | | | | | | | | |
| | Block | | | | | | | | | |
| 498 | Method | | | | | | | 20% | T3P Method A | |
| | | | | | | | | TFA | | |
| | Building | | | | | | | | | |
| | Block | | | | | | | | | |
| 499 | Method | | | | | | | 20% | T3P Method A | |
| | | | | | | | | TFA | | |
| | Building | | | | | | | | | |
| | Block | | | | | | | | | |
| 500 | Method | HATUnf | | | | | | 20% | T3P Method B | |
| | | | | | | | | TFA | | |
| | Building | Acd0588 | | | | | | | | |
| | Block | | | | | | | | | |
| 501 | Method | HATUnf | | | | | | 20% | T3P Method B | |
| | | | | | | | | TFA | | |
| | Building | Acd0588 | | | | | | | | |
| | Block | | | | | | | | | |
| 502 | Method | HATUnf | | | | | | 20% | T3P Method B | |
| | | | | | | | | TFA | | |
| | Building | Acd0588 | | | | | | | | |
| | Block | | | | | | | | | |
| 503 | Method | HATUnf | | | | | | 20% | T3P Method A | |
| | | | | | | | | TFA | | |
| | Building | Acd0317 | | | | | | | | |
| | Block | | | | | | | | | |
| 504 | Method | HATUnf | | | | | | 20% | T3P Method A | |
| | | | | | | | | TFA | | |
| | Building | Acd0317 | | | | | | | | |
| | Block | | | | | | | | | |
| 505 | Method | HATUnf | | | | | | 20% | T3P Method B | |
| | | | | | | | | TFA | | |
| | Building | Acd0588 | | | | | | | | |
| | Block | | | | | | | | | |
| 506 | Method | HATUnf | | | | | | 20% | T3P Method B | |
| | | | | | | | | TFA | | |
| | Building | Acd0540 | | | | | | | | |
| | Block | | | | | | | | | |
| 507 | Method | HATUnf | | | | | | 20% | T3P Method B | |
| | | | | | | | | TFA | | |
| | Building | Acd0588 | | | | | | | | |
| | Block | | | | | | | | | |
| 508 | Method | HATUnf | | | | | | 20% | T3P Method B | |
| | | | | | | | | TFA | | |
| | Building | Acd0540 | | | | | | | | |
| | Block | | | | | | | | | |
| 509 | Method | HATUnf | | | | | | 20% | T3P Method A | |
| | | | | | | | | TFA | | |
| | Building | Acd0317 | | | | | | | | |
| | Block | | | | | | | | | |
| 510\| | Method | HATUnf | | | | | | 20% | T3P Method A | |
| | | | | | | | | TFA | | |
| | Building | Acd0317 | | | | | | | | |
| | Block | | | | | | | | | |
| 511 | Method | HATUnf | | | | | | 20% | T3P Method A | |
| | | | | | | | | TFA | | |
| | Building | Acd0540 | | | | | | | | |
| | Block | | | | | | | | | |
| 512 | Method | HATUnf | | | | | | 20% | T3P Method A | |
| | | | | | | | | TFA | | |
| | Building | Acd0540 | | | | | | | | |
| | Block | | | | | | | | | |
| 513 | Method | HATUnf | | | | | | 20% | T3P Method A | |
| | | | | | | | | TFA | | |
| | Building | Acd0540 | | | | | | | | |
| | Block | | | | | | | | | |
| 514 | Method | HATUnf | | | | | | 20% | T3P Method A | |
| | | | | | | | | TFA | | |
| | Building | Acd0540 | | | | | | | | |
| | Block | | | | | | | | | |
| 515 | Method | HATUnf | | | | | | 20% | T3P Method B | |
| | | | | | | | | TFA | | |
| | Building | Acd0540 | | | | | | | | |
| | Block | | | | | | | | | |
| 516 | Method | HATUnf | | | | | | 20% | T3P Method B | |
| | | | | | | | | TFA | | |
| | Building | Acd0588 | | | | | | | | |
| | Block | | | | | | | | | |
| 517 | Method | HATUnf | | DdeR | MITS | | | 24% | T3P Method A | |
| | | | | | | | | HFIP | | |
| | Building | Acd0486 | | | MeOH* | | | | | |
| | Block | | | | | | | | | |
| 518 | Method | HATUnf | | | | | | 20% | T3P Method A | |
| | | | | | | | | TFA | | |
| | Building | Acd0588 | | | | | | | | |
| | Block | | | | | | | | | |
| 519 | Method | HATUnf | | | | | | 20% | T3P Method B | |
| | | | | | | | | TFA | | |
| | Building | Acd0540 | | | | | | | | |
| | Block | | | | | | | | | |
| 520 | Method | HATUnf | | | | | | 20% | T3P Method B | |
| | | | | | | | | TFA | | |
| | Building | Acd0505 | | | | | | | | |
| | Block | | | | | | | | | |
| 521 | Method | HATUnf | | | | | | 20% | T3P Method B | |
| | | | | | | | | TFA | | |
| | Building | Acd0317 | | | | | | | | |
| | Block | | | | | | | | | |
| 522 | Method | HATUnf | | | | | | 20% | T3P Method B | |
| | | | | | | | | TFA | | |
| | Building | Acd0505 | | | | | | | | |
| | Block | | | | | | | | | |
| 523 | Method | | | | | | | 20% | T3P Method A | |
| | | | | | | | | TFA | | |
| | Building | | | | | | | | | |
| | Block | | | | | | | | | |
| 524 | Method | HATUnf | | | | | | 20% | T3P Method B | |
| | | | | | | | | TFA | | |
| | Building | Acd0540 | | | | | | | | |
| | Block | | | | | | | | | |
| 525 | Method | HATUnf | | | | | | 20% | T3P Method B | |
| | | | | | | | | TFA | | |
| | Building | Acd0505 | | | | | | | | |
| | Block | | | | | | | | | |
| 526 | Method | HATUnf | | | | | | 20% | T3P Method B | |
| | | | | | | | | TFA | | |
| | Building | Acd0540 | | | | | | | | |
| | Block | | | | | | | | | |
| 527 | Method | HATUnf | | DdeR | | | | 24% | T3P Method B | |
| | | | | | | | | HFIP | | |
| | Building | Acd0505 | | | | | | | | |
| | Block | | | | | | | | | |
| 528 | Method | | | | | | | 20% | T3P Method A | |
| | | | | | | | | TFA | | |
| | Building | | | | | | | | | |
| | Block | | | | | | | | | |
| 529 | Method | | | | | | | 20% | T3P Method A | |
| | | | | | | | | TFA | | |
| | Building | | | | | | | | | |
| | Block | | | | | | | | | |
| 530 | Method | KO | | | | | | 20% | T3P Method A | |
| | | | | | | | | TFA | | |
| | Building | Acd0504 | | | | | | | | |
| | Block | | | | | | | | | |
| 531 | Method | KO | | | | | | 20% | T3P Method A | |
| | | | | | | | | TFA | | |
| | Building | Acd0504 | | | | | | | | |
| | Block | | | | | | | | | |
| 532 | Method | HATUnf | | | | | | 20% | T3P Method A | |
| | | | | | | | | TFA | | |
| | Building | Acd0540 | | | | | | | | |
| | Block | | | | | | | | | |
| 533 | Method | HATUnf | | | | | | 20% | T3P Method A | |
| | | | | | | | | TFA | | |
| | Building | Acd0540 | | | | | | | | |
| | Block | | | | | | | | | |
| 534 | Method | HATUnf | | | | | | 20% | T3P Method A | |
| | | | | | | | | TFA | | |
| | Building | Acd0540 | | | | | | | | |
| | Block | | | | | | | | | |
| 535 | Method | HATUnf | | | | | | 20% | T3P Method A | |
| | | | | | | | | TFA | | |
| | Building | Acd0588 | | | | | | | | |
| | Block | | | | | | | | | |
| 536 | Method | HATUnf | | | | | | 20% | T3P Method A | |
| | | | | | | | | TFA | | |
| | Building | Acd0588 | | | | | | | | |
| | Block | | | | | | | | | |
| 537 | Method | HATUnf | | | | | | 20% | T3P Method A | |
| | | | | | | | | TFA | | |
| | Building | Acd0588 | | | | | | | | |
| | Block | | | | | | | | | |
| 538 | Method | HATUnf | | | | | | 20% | T3P Method A | |
| | | | | | | | | TFA | | |
| | Building | Acd0588 | | | | | | | | |
| | Block | | | | | | | | | |
| 539 | Method | HATUnf | | | | | | 20% | T3P Method A | |
| | | | | | | | | TFA | | |
| | Building | Acd0588 | | | | | | | | |
| | Block | | | | | | | | | |
| 540 | Method | HATUnf | | | | | | 20% | T3P Method A | |
| | | | | | | | | TFA | | |
| | Building | Acd0588 | | | | | | | | |
| | Block | | | | | | | | | |
| 541 | Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 542 | \|Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 543 | Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 544 | Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 545 | Method | HATUnf | | | | | | 20% | T3P Method A | |
| | | | | | | | | TFA | | |
| | Building | Acd0505 | | | | | | | | |
| | Block | | | | | | | | | |
| 546 | Method | HATUnf | | | | | | 20% | T3P Method B | |
| | | | | | | | | TFA | | |
| | Building | Acd0317 | | | | | | | | |
| | Block | | | | | | | | | |
| 547 | Method | HATUnf | | DdeR | | | | 24 % | T3P Method B | |
| | | | | | | | | HFIP | | |
| | Building | Acd0486 | | | | | | | | |
| | Block | | | | | | | | | |
| 548 | Method | HATUnf | | | | | | 20% | T3P Method B | |
| | | | | | | | | TFA | | |
| | Building | Acd0588 | | | | | | | | |
| | Block | | | | | | | | | |
| 549 | Method | HATUnf | | DdeR | MITS | | | 24% | PyBop Method | |
| | | | | | | | | HFIP | A | |
| | Building | Acd0486 | | | MeOH | | | | | |
| | Block | | | | | | | | | |
| 550 | Method | HATUnf | | | | | | 20% | T3P Method A | |
| | | | | | | | | TFA | | |
| | Building | Acd0540 | | | | | | | | |
| | Block | | | | | | | | | |
| 551 | Method | HATUnf | | | | | | 20% | T3P Method A | |
| | | | | | | | | TFA | | |
| | Building | Acd0540 | | | | | | | | |
| | Block | | | | | | | | | |
| 552 | Method | HATUnf | | | | | | 20% | PyBop Method | |
| | | | | | | | | TFA | B | |
| | Building | Acd0588 | | | | | | | | |
| | Block | | | | | | | | | |
| 553 | Method | HATUnf | | DdeR | | | | 24% | T3P Method B | |
| | | | | | | | | HFIP | | |
| | Building | Acd0486 | | | | | | | | |
| | Block | | | | | | | | | |
| 554 | Method | | | | | | | 20% | T3P Method A | |
| | | | | | | | | TFA | | |
| | Building | | | | | | | | | |
| | Block | | | | | | | | | |
| 555 | Method | | | | | | | 20% | T3P Method A | |
| | | | | | | | | TFA | | |
| | Building | | | | | | | | | |
| | Block | | | | | | | | | |
| 556 | Method | | | | | | | 20% | T3P Method A | |
| | | | | | | | | TFA | | |
| | Building | | | | | | | | | |
| | Block | | | | | | | | | |
| 557 | Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 558 | Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 559 | Method | HATUnf | | | | | | 20% | T3P Method B | |
| | | | | | | | | TFA | | |
| | Building | Acd0588 | | | | | | | | |
| | Block | | | | | | | | | |
| 560 | Method | | | | | | | 20% | T3P Method A | |
| | | | | | | | | TFA | | |
| | Building | | | | | | | | | |
| | Block | | | | | | | | | |
| 561 | Method | HATUnf | | | | | | 20% | T3P Method A | |
| | | | | | | | | TFA | | |
| | Building | Acd0532 | | | | | | | | |
| | Block | | | | | | | | | |
| 562 | Method | HATUnf | | | | | | 20% | T3P Method A | |
| | | | | | | | | TFA | | |
| | Building | Acd0317 | | | | | | | | |
| | Block | | | | | | | | | |
| 563 | Method | HATUnf | | | | | | 20% | T3P Method B | |
| | | | | | | | | TFA | | |
| | Building | Acd0588 | | | | | | | | |
| | Block | | | | | | | | | |
| 564 | Method | HATUnf | | | | | | 20% | T3P Method A | |
| | | | | | | | | TFA | | |
| | Building | Acd0540 | | | | | | | | |
| | Block | | | | | | | | | |
| 565 | Method | HATUnf | | | | | | 20% | T3P Method A | |
| | | | | | | | | TFA | | |
| | Building | Acd0540 | | | | | | | | |
| | Block | | | | | | | | | |
| 566 | Method | HATUnf | | | | | | 20% | T3P Method A | |
| | | | | | | | | TFA | | |
| | Building | Acd0588 | | | | | | | | |
| | Block | | | | | | | | | |
| 567 | Method | HATUnf | | | | | | 20% | T3P Method A | |
| | | | | | | | | TFA | | |
| | Building | Acd0588 | | | | | | | | |
| | Block | | | | | | | | | |
| 568 | Method | HATUnf | | | | | | 20% | T3P Method A | |
| | | | | | | | | TFA | | |
| | Building | Acd0588 | | | | | | | | |
| | Block | | | | | | | | | |
| 569 | Method | HATUnf | | | | | | 20% | T3P Method A | |
| | | | | | | | | TFA | | |
| | Building | Acd0588 | | | | | | | | |
| | Block | | | | | | | | | |
| 570 | Method | HATUnf | | | | | | 20% | T3P Method A | |
| | | | | | | | | TFA | | |
| | Building | Acd0588 | | | | | | | | |
| | Block | | | | | | | | | |
| 571 | Method | HATUnf | | | | | | 20% | T3P Method A | |
| | | | | | | | | TFA | | |
| | Building | Acd0540 | | | | | | | | |
| | Block | | | | | | | | | |
| 572 | Method | HATUnf | | | | | | 20% | T3P Method A | |
| | | | | | | | | TFA | | |
| | Building | Acd0594 | | | | | | | | |
| | Block | | | | | | | | | |
| 573 | Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 574 | Method | HATUnf | | | | | | 20% | T3P Method A | |
| | | | | | | | | TFA | | |
| | Building | Acd0588 | | | | | | | | |
| | Block | | | | | | | | | |
| 575 | Method | HATUnf | | | | | | 20% | T3P Method A | |
| | | | | | | | | TFA | | |
| | Building | Acd0540 | | | | | | | | |
| | Block | | | | | | | | | |
| 576 | Method | HATUnf | | | | | | 20% | T3P Method A | |
| | | | | | | | | TFA | | |
| | Building | Acd0588 | | | | | | | | |
| | Block | | | | | | | | | |
| 577 | Method | HATUnf | | DdeR | MITS | | | 24% | PyBop Method | |
| | | | | | | | | HFIP | A | |
| | Building | Acd0486 | | | MeOH | | | | | |
| | Block | | | | | | | | | |
| 578 | Method | | | | | | | 20% | T3P Method A | |
| | | | | | | | | TFA | | |
| | Building | | | | | | | | | |
| | Block | | | | | | | | | |
| 579 | Method | HATUnf | | DdeR | MITS | | | 24% | T3P Method A | |
| | | | | | | | | HFIP | | |
| | Building | Acd0486 | | | MeOH | | | | | |
| | Block | | | | | | | | | |
| 580 | Method | HATUnf | | | | | | 20% | T3P Method A | |
| | | | | | | | | TFA | | |
| | Building | Acd0505 | | | | | | | | |
| | Block | | | | | | | | | |
| 581 | Method | HATUnf | | | | | | 20% | T3P Method A | |
| | | | | | | | | TFA | | |
| | Building | Acd0747 | | | | | | | | |
| | Block | | | | | | | | | |
| 582 | Method | HATUnf | | | | | | 20% | T3P Method A | |
| | | | | | | | | TFA | | |
| | Building | Acd0540 | | | | | | | | |
| | Block | | | | | | | | | |
| 583 | Method | HATUnf | | | | | | 20% | T3P Method A | |
| | | | | | | | | TFA | | |
| | Building | Acd0540 | | | | | | | | |
| | Block | | | | | | | | | |
| 584 | Method | HATUnf | | | | | | 20% | T3P Method B | |
| | | | | | | | | TFA | | |
| | Building | Acd0540 | | | | | | | | |
| | Block | | | | | | | | | |
| 585 | Method | HATUnf | | | | | | 20% | T3P Method B | |
| | | | | | | | | TFA | | |
| | Building | Acd0588 | | | | | | | | |
| | Block | | | | | | | | | |
| 586 | Method | HATUnf | | | | | | 20% | T3P Method B | |
| | | | | | | | | TFA | | |
| | Building | Acd0540 | | | | | | | | |
| | Block | | | | | | | | | |
| 587 | Method | HATUnf | | | | | | 20% | T3P Method B | |
| | | | | | | | | TFA | | |
| | Building | Acd0588 | | | | | | | | |
| | Block | | | | | | | | | |
| 588 | Method | HATUnf | | | | | | 20% | T3P Method A | |
| | | | | | | | | TFA | | |
| | Building | Acd0747 | | | | | | | | |
| | Block | | | | | | | | | |
| 589 | Method | HATUnf | | | | | | 20% | T3P Method A | |
| | | | | | | | | TFA | | |
| | Building | Acd0540 | | | | | | | | |
| | Block | | | | | | | | | |
| 590 | Method | HATUnf | | | | | | 20% | T3P Method B | |
| | | | | | | | | TFA | | |
| | Building | Acd0540 | | | | | | | | |
| | Block | | | | | | | | | |
| 591 | Method | HATUnf | | | | | | 20% | T3P Method B | |
| | | | | | | | | TFA | | |
| | Building | Acd0540 | | | | | | | | |
| | Block | | | | | | | | | |
| 592 | Method | HATUnf | | | | | | 20% | T3P Method B | |
| | | | | | | | | TFA | | |
| | Building | Acd0540 | | | | | | | | |
| | Block | | | | | | | | | |
| 593 | Method | HATUnf | | | | | | 20% | T3P Method B | |
| | | | | | | | | TFA | | |
| | Building | Acd0540 | | | | | | | | |
| | Block | | | | | | | | | |
| 594 | Method | HATUnf | | | | | | 20% | T3P Method B | |
| | | | | | | | | TFA | | |
| | Building | Acd0540 | | | | | | | | |
| | Block | | | | | | | | | |
| 595 | Method | HATUnf | | | | | | 20% | T3P Method B | |
| | | | | | | | | TFA | | |
| | Building | Acd0540 | | | | | | | | |
| | Block | | | | | | | | | |
| 596 | Method | HATUnf | | | | | | 20% | T3P Method B | |
| | | | | | | | | TFA | | |
| | Building | Acd0540 | | | | | | | | |
| | Block | | | | | | | | | |
| 597 | Method | HATUnf | | | | | | 20% | T3P Method B | |
| | | | | | | | | TFA | | |
| | Building | Acd0540 | | | | | | | | |
| | Block | | | | | | | | | |
| 598 | Method | HATUnf | | | | | | 20% | T3P Method B | |
| | | | | | | | | TFA | | |
| | Building | Acd0540 | | | | | | | | |
| | Block | | | | | | | | | |
| 599 | Method | | | DdeR | | | | 24% | T3P Method B | tBu |
| | | | | | | | | HFIP | | |
| | Building | | | | | | | | | |
| | Block | | | | | | | | | |
| 600 | Method | | | DdeR | | | | 24% | T3P Method B | tBu |
| | | | | | | | | HFIP | | |
| | Building | | | | | | | | | |
| | Block | | | | | | | | | |
| 601 | Method | | | DdeR | MITS | | | 24% | T3P Method A | ItBu |
| | | | | | | | | HFIP | | |
| | Building | | | | MeOH | | | | | |
| | Block | | | | | | | | | |
| 602 | Method | HATUnf | | | | | | 20% | T3P Method B | |
| | | | | | | | | TFA | | |
| | Building | Acd0588 | | | | | | | | |
| | Block | | | | | | | | | |
| 603 | Method | HATUnf | | DdeR | | | | 24% | T3P Method B | tBu |
| | | | | | | | | HFIP | | |
| | Building | Acd0588 | | | | | | | | |
| | Block | | | | | | | | | |
| 604 | Method | HATUnf | | DdeR | | | | 24% | T3P Method B | |
| | | | | | | | | HFIP | | |
| | Building | Acd0486 | | | | | | | | |
| | Block | | | | | | | | | |
| 605 | Method | HATUnf | | DdeR | | | | 24% | T3P Method B | |
| | | | | | | | | HFIP | | |
| | Building | Acd0486 | | | | | | | | |
| | Block | | | | | | | | | |
| 606 | Method | HATUnf | | DdeR | | | | 24% | T3P Method B | |
| | | | | | | | | HFIP | | |
| | Building | Acd0486 | | | | | | | | |
| | Block | | | | | | | | | |
| 607 | Method | HATUnf | | DdeR | | | | 24% | T3P Method B | |
| | | | | | | | | HFIP | | |
| | Building | Acd0486 | | | | | | | | |
| | Block | | | | | | | | | |
| 608 | Method | HATUnf | | DdeR | | | | 24% | T3P Method B | tBu |
| | | | | | | | | HFIP | | |
| | Building | Acd0588 | | | | | | | | |
| | Block | | | | | | | | | |
| 609 | Method | HATUnf | | DdeR | | | | 24% | T3P Method B | tBu |
| | | | | | | | | HFIP | | |
| | Building | Acd0486 | | | | | | | | |
| | Block | | | | | | | | | |
| 610 | Method | HATUnf | | DdeR | | | | 24% | T3P Method B | |
| | | | | | | | | HFIP | | |
| | Building | Acd0317 | | | | | | | | |
| | Block | | | | | | | | | |
| 611 | Method | HATUnf | | | | | | 20% | PyBop Method | |
| | | | | | | | | TFA | B | |
| | Building | Acd0588 | | | | | | | | |
| | Block | | | | | | | | | |
| 612 | Method | HATUnf | | DdeR | | | | 24% | T3P Method B | tBu |
| | | | | | | | | HFIP | | |
| | Building | Acd0588 | | | | | | | | |
| | Block | | | | | | | | | |
| 613 | Method | HATUnf | | DdeR | MITS | | | 24% | T3P Method B | tBu |
| | | | | | | | | HFIP | | |
| | Building | Acd0588 | | | MeOH | | | | | |
| | Block | | | | | | | | | |
| 614 | Method | HATUnf | | DdeR | MITS | | | 24% | T3P Method A | tBu |
| | | | | | | | | HFIP | | |
| | Building | Acd0588 | | | MeOH | | | | | |
| | Block | | | | | | | | | |
| 615 | Method | HATUnf | | DdeR | | Morph | | 20% | T3P Method A | |
| | | | | | | | | TFA | | |
| | Building | Acd0588 | | | | B2BE | | | | |
| | Block | | | | | | | | | |
| 616 | Method | HATUnf | | DdeR | | | | 24% | T3P Method A | |
| | | | | | | | | HFIP | | |
| | Building | Acd0588 | | | | | | | | |
| | Block | | | | | | | | | |
| 617 | Method | HATUnf | | | | | | 20% | T3P Method B | |
| | | | | | | | | TFA | | |
| | Building | Acd0503 | | | | | | | | |
| | Block | | | | | | | | | |
| 618 | Method | HATUnf | | | | | | 20% | T3P Method B | |
| | | | | | | | | TFA | | |
| | Building | Acd0503 | | | | | | | | |
| | Block | | | | | | | | | |
| 619 | Method | HATUnf | | | | | | 20% | T3P Method B | |
| | | | | | | | | TFA | | |
| | Building | Acd0540 | | | | | | | | |
| | Block | | | | | | | | | |
| 620 | Method | HATUnf | | | | | | 20% | T3P Method B | |
| | | | | | | | | TFA | | |
| | Building | Acd0588 | | | | | | | | |
| | Block | | | | | | | | | |
| 621 | Method | HATUnf | | | | | | 20% | T3P Method B | |
| | | | | | | | | TFA | | |
| | Building | Acd0540 | | | | | | | | |
| | Block | | | | | | | | | |
| 622 | \|Method | | | | | | | 20% | T3P Method A | |
| | | | | | | | | TFA | | |
| | Building | | | | | | | | | |
| | Block | | | | | | | | | |
| 623 | Method | HATUnf | | DdeR | | | | 24% | T3P Method B | |
| | | | | | | | | HFIP | | |
| | Building | Acd0486 | | | | | | | | |
| | Block | | | | | | | | | |
| 624 | Method | HATUnf | | | | | | 20% | T3P Method B | |
| | | | | | | | | TFA | | |
| | Building | Acd0540 | | | | | | | | |
| | Block | | | | | | | | | |
| 625 | Method | | | DdeR | MITS | | | 24% | T3P Method A | |
| | | | | | | | | HFIP | | |
| | Building | | | | MeOH | | | | | |
| | Block | | | | | | | | | |
| 626 | Method | HATUnf | | | | | | 20% | T3P Method B | |
| | | | | | | | | TFA | | |
| | Building | Acd0540 | | | | | | | | |
| | Block | | | | | | | | | |
| 627 | Method | HATUnf | | | | | | 20% | T3P Method B | |
| | | | | | | | | TFA | | |
| | Building | Acd0540 | | | | | | | | |
| | Block | | | | | | | | | |
| 628 | Method | HATUnf | | | | | | 20% | T3P Method B | |
| | | | | | | | | TFA | | |
| | Building | Acd0540 | | | | | | | | |
| | Block | | | | | | | | | |
| 629 | Method | HATUnf | | | | | | 20% | T3P Method B | |
| | | | | | | | | TFA | | |
| | Building | Acd0540 | | | | | | | | |
| | Block | | | | | | | | | |
| 630 | Method | HATUnf | | DdeR | | | | 24% | T3P Method B | |
| | | | | | | | | HFIP | | |
| | Building | Acd0486 | | | | | | | | |
| | Block | | | | | | | | | |
| 631 | Method | HATUnf | | DdeR | | | | 24% | PyBop Method | |
| | | | | | | | | HFIP | B | |
| | Building | Acd0486 | | | | | | | | |
| | Block | | | | | | | | | |
| 632 | Method | HATUnf | | DdeR | | | | 24% | T3P Method B | |
| | | | | | | | | HFIP | | |
| | Building | Acd0486 | | | | | | | | |
| | Block | | | | | | | | | |
| 633 | Method | HATUnf | | | | | | 20% | T3P Method A | |
| | | | | | | | | TFA | | |
| | Building | Acd0505 | | | | | | | | |
| | Block | | | | | | | | | |
| 634 | Method | HATUnf | | | | | | 20% | T3P Method A | |
| | | | | | | | | TFA | | |
| | Building | Acd0747 | | | | | | | | |
| | Block | | | | | | | | | |
| 635 | Method | HATUnf | | DdeR | | | | 24% | T3P Method B | |
| | | | | | | | | HFIP | | |
| | Building | Acd0805 | | | | | | | | |
| | Block | | | | | | | | | |
| 636 | Method | HATUnf | | DdeR | | | | 24% | T3P Method B | |
| | | | | | | | | HFIP | | |
| | Building | Acd0807 | | | | | | | | |
| | Block | | | | | | | | | |
| 637 | Method | HATUnf | | DdeR | MITS | | | 24% | T3P Method A | |
| | | | | | | | | HFIP | | |
| | Building | Acd0532 | | | MeOH | | | | | |
| | Block | | | | | | | | | |
| 638 | Method | | | DdeR | | | | 24% | T3P Method A | |
| | | | | | | | | HFIP | | |
| | Building | | | | | | | | | |
| | Block | | | | | | | | | |
| 639 | Method | HATUnf | | | | | | 20% | T3P Method A | |
| | | | | | | | | TFA | | |
| | Building | Acd0588 | | | | | | | | |
| | Block | | | | | | | | | |
| 640 | Method | HATUnf | | | | | | 20% | T3P Method A | |
| | | | | | | | | TFA | | |
| | Building | Acd0505 | | | | | | | | |
| | Block | | | | | | | | | |
| 641 | Method | HATUnf | | | | | | 20% | T3P Method A | |
| | | | | | | | | TFA | | |
| | Building | Acd0809 | | | | | | | | |
| | Block | | | | | | | | | |
| 642 | Method | HATUnf | | | | | | 20% | T3P Method B | |
| | | | | | | | | TFA | | |
| | Building | Acd0809 | | | | | | | | |
| | Block | | | | | | | | | |
| 643 | Method | HATUnf | | | | | | 20% | T3P Method A | |
| | | | | | | | | TFA | | |
| | Building | Acd0810 | | | | | | | | |
| | Block | | | | | | | | | |
| 644 | Method | HATUnf | | | | | | 20% | T3P Method B | |
| | | | | | | | | TFA | | |
| | Building | Acd0810 | | | | | | | | |
| | Block | | | | | | | | | |
| 645 | Method | HATUnf | | | | | | 20% | T3P Method A | |
| | | | | | | | | TFA | | |
| | Building | Acd0811 | | | | | | | | |
| | Block | | | | | | | | | |
| 646 | Method | HATUnf | | | | | | 20% | T3P Method B | |
| | | | | | | | | TFA | | |
| | Building | Acd0811 | | | | | | | | |
| | Block | | | | | | | | | |
| 647 | Method | HATUnf | | DdeR | MITS | | | 24% | T3P Method A | |
| | | | | | | | | HFIP | | |
| | Building | Acd0813 | | | MeOH | | | | | |
| | Block | | | | | | | | | |
| 648 | Method | HATUnf | | | | | | 20% | T3P Method B | |
| | | | | | | | | TFA | | |
| | Building | Acd0588 | | | | | | | | |
| | Block | | | | | | | | | |
| 649 | Method | HATUnf | | DdeR | | | | 24% | PyBop Method | |
| | | | | | | | | HFIP | A | |
| | Building | Acd0486 | | | | | | | | |
| | Block | | | | | | | | | |
| 650 | Method | HATUnf | | | | | | 20% | T3P Method B | |
| | | | | | | | | TFA | | |
| | Building | Acd0588 | | | | | | | | |
| | Block | | | | | | | | | |
| 651 | Method | HATUnf | | | | | | 20% | T3P Method B | |
| | | | | | | | | TFA | | |
| | Building | Acd0588 | | | | | | | | |
| | Block | | | | | | | | | |
| 652 | Method | HATUnf | | | | | | 20% | T3P Method B | |
| | | | | | | | | TFA | | |
| | Building | Acd0540 | | | | | | | | |
| | Block | | | | | | | | | |
| 653 | Method | HATUnf | | | | | | 20% | T3P Method B | |
| | | | | | | | | TFA | | |
| | Building | Acd0540 | | | | | | | | |
| | Block | | | | | | | | | |
| 654 | Method | HATUnf | | | | | | 20% | T3P Method B | |
| | | | | | | | | TFA | | |
| | Building | Acd0540 | | | | | | | | |
| | Block | | | | | | | | | |
| 655 | Method | HATUnf | | | | | | 20% | T3P Method B | |
| | | | | | | | | TFA | | |
| | Building | Acd0540 | | | | | | | | |
| | Block | | | | | | | | | |
| 656 | Method | HATUnf | | | | | | 20% | T3P Method B | |
| | | | | | | | | TFA | | |
| | Building | Acd0540 | | | | | | | | |
| | Block | | | | | | | | | |
| 657 | Method | HATUnf | | | | | | 20% | T3P Method B | |
| | | | | | | | | TFA | | |
| | Building | Acd0540 | | | | | | | | |
| | Block | | | | | | | | | |
| 658 | Method | HATUnf | | | | | | 20% | T3P Method B | |
| | | | | | | | | TFA | | |
| | Building | Acd0540 | | | | | | | | |
| | Block | | | | | | | | | |
| 659 | Method | HATUnf | | | | | | 20% | T3P Method B | |
| | | | | | | | | TFA | | |
| | Building | Acd0540 | | | | | | | | |
| | Block | | | | | | | | | |
| 660 | Method | HATUnf | | | | | | 20% | T3P Method B | |
| | | | | | | | | TFA | | |
| | Building | Acd0588 | | | | | | | | |
| | Block | | | | | | | | | |
| 661 | Method | HATUnf | | | | | | 20% | T3P Method B | |
| | | | | | | | | TFA | | |
| | Building | Acd0588 | | | | | | | | |
| | Block | | | | | | | | | |
| 662 | Method | HATUnf | | | | | | 20% | T3P Method B | |
| | | | | | | | | TFA | | |
| | Building | Acd0540 | | | | | | | | |
| | Block | | | | | | | | | |
| 663 | Method | HATUnf | | | | | | 20% | T3P Method A | |
| | | | | | | | | TFA | | |
| | Building | Acd0540 | | | | | | | | |
| | Block | | | | | | | | | |
| 664 | Method | HATUnf | | | | | | 20% | T3P Method B | |
| | | | | | | | | TFA | | |
| | Building | Acd0540 | | | | | | | | |
| | Block | | | | | | | | | |
| 665 | Method | HATUnf | | | | | | 20% | T3P Method B | |
| | | | | | | | | TFA | | |
| | Building | Acd0540 | | | | | | | | |
| | Block | | | | | | | | | |
| 666 | Method | HATUnf | | | | | | 20% | T3P Method B | |
| | | | | | | | | TFA | | |
| | Building | Acd0540 | | | | | | | | |
| | Block | | | | | | | | | |
| 667 | Method | HATUnf | | | | | | 20% | T3P Method B | |
| | | | | | | | | TFA | | |
| | Building | Acd0540 | | | | | | | | |
| | Block | | | | | | | | | |
| 668 | Method | HATUnf | | | | | | 20% | T3P Method B | |
| | | | | | | | | TFA | | |
| | Building | Acd0540 | | | | | | | | |
| | Block | | | | | | | | | |
| 669 | Method | HATUnf | | | | | | 20% | T3P Method B | |
| | | | | | | | | TFA | | |
| | Building | Acd0540 | | | | | | | | |
| | Block | | | | | | | | | |
| 670 | Method | HATUnf | | | | | | 20% | T3P Method B | |
| | | | | | | | | TFA | | |
| | Building | Acd0540 | | | | | | | | |
| | Block | | | | | | | | | |
| 671 | Method | HATUnf | | | | | | 20% | T3P Method B | |
| | | | | | | | | TFA | | |
| | Building | Acd0540 | | | | | | | | |
| | Block | | | | | | | | | |
| 672 | Method | HATUnf | | | | | | 20% | T3P Method B | |
| | | | | | | | | TFA | | |
| | Building | Acd0540 | | | | | | | | |
| | Block | | | | | | | | | |
| 673 | Method | HATUnf | | | | | | 20% | T3P Method B | |
| | | | | | | | | TFA | | |
| | Building | Acd0540 | | | | | | | | |
| | Block | | | | | | | | | |
| 674 | Method | HATUnf | | | | | | 20% | T3P Method B | |
| | | | | | | | | TFA | | |
| | Building | Acd0540 | | | | | | | | |
| | Block | | | | | | | | | |
| 675 | Method | HATUnf | | | | | | 20% | T3P Method B | |
| | | | | | | | | TFA | | |
| | Building | Acd0540 | | | | | | | | |
| | Block | | | | | | | | | |
| 676 | Method | | | DdeR | | | | 24% | T3P Method B | |
| | | | | | | | | HFIP | | |
| | Building | | | | | | | | | |
| | Block | | | | | | | | | |
| 677 | Method | HATUnf | | DdeR | | Morph | | 24% | T3P Method B | |
| | | | | | | | | HFIP | | |
| | Building | Acd0805 | | | | B2BE | | | | |
| | Block | | | | | | | | | |
| 678 | Method | HATUnf | | DdeR | | Morph | | 24% | T3P Method B | |
| | | | | | | | | HFIP | | |
| | Building | Acd0505 | | | | B2BE | | | | |
| | Block | | | | | | | | | |
| 679 | Method | HATUnf | | DdeR | | Morph | | 24% | T3P Method B | |
| | | | | | | | | HFIP | | |
| | Building | Acd0486 | | | | B2BE | | | | |
| | Block | | | | | | | | | |
| 680 | Method | HATUnf | | | | | | | | |
| | Building | Acd0588 | | | | | | 20% | T3P Method B | |
| | Block | | | | | | | TFA | | |

*For Example 517: The conditions used for methylation of the sidechain of Residue 6 in Example 517, T13 concomitantly formed the methyl ether on what was previously the hydroxyl group on Residue 3.

Table 3, below, identifies the expected and observed mass spectrometry data for each exemplary compound in Table 2A and B. The first two analytical columns list the expected and observed mass spectrometry results of the linear intermediate after it was cleaved from the resin, but before cyclization. The last two columns on the right list the expected and observed mass spectrometry results of the cyclized final product.

TABLE 3
Analytical Data for Exemplary Compounds of Table 2A and B

| | Expected | | | |
|---|---|---|---|---|
| | for linear | Observed for | Expected | Observed |
| | peptide | linear peptide | compound | compound |
| | Intermediate | Intermediate | Formula I | Formula I |
| Ex. | M + H | M/z | M + H | M/z |
| 1 | 1089.70 | 1091.10 | 1071.69 | 1071.60 |
| 2 | 967.52 | 967.50 | 949.51 | 949.60 |
| 3 | 991.52 | 991.59 | 973.51 | 973.56 |
| 4 | 894.39 | 894.30 | 876.38 | 876.40 |
| 5 | 985.59 | 985.50 | 967.58 | 967.60 |
| 6 | 915.55 | 915.45 | 897.54 | 897.60 |
| 7 | 943.54 | 943.50 | 925.53 | 925.50 |
| 8 | 949.47 | 949.50 | 931.46 | 931.60 |
| 9 | 1076.57 | 1076.55 | 1058.56 | 1058.60 |
| 10 | 977.50 | 977.40 | 959.49 | 959.60 |
| 11 | 963.49 | 962.85 | 945.48 | 945.60 |
| 12 | 977.50 | 977.40 | 959.49 | 959.60 |
| 13 | 1005.53 | 1005.45 | 987.52 | 987.60 |
| 14 | 1019.55 | 1019.55 | 1001.54 | 1001.60 |
| 15 | 1005.53 | 1005.45 | 987.52 | 987.60 |
| 16 | 1003.52 | 1003.50 | 985.51 | 985.60 |
| 17 | 989.50 | 989.40 | 971.49 | 971.60 |
| 18 | 991.52 | 991.50 | 973.51 | 973.60 |
| 19 | 971.57 | 971.55 | 953.56 | 953.55 |
| 20 | 1098.67 | 1098.60 | 1080.66 | 1080.66 |
| 21 | 1005.53 | 1005.45 | 987.52 | 987.50 |
| 22 | 1005.53 | 1005.45 | 987.52 | 987.60 |
| 23 | 1035.51 | 1035.45 | 1017.50 | 1017.60 |
| 24 | 997.57 | 997.50 | 979.55 | 979.60 |
| 25 | 991.52 | 991.50 | 973.51 | 973.60 |
| 26 | 1019.55 | 1019.55 | 1001.54 | 1001.60 |
| 27 | 886.48 | 886.50 | 868.47 | 868.60 |
| 28 | 1043.56 | 1043.55 | 1025.54 | 1025.60 |
| 29 | 926.49 | 926.55 | 908.48 | 908.60 |
| 30 | 840.42 | 840.45 | 822.41 | 822.40 |
| 31 | 826.40 | 826.35 | 808.39 | 808.40 |
| 32 | 977.50 | 977.40 | 959.49 | 959.60 |
| 33 | 979.48 | 979.50 | 961.47 | 961.40 |
| 34 | 963.60 | 963.60 | 945.59 | 945.60 |
| 35 | 1009.49 | 1009.50 | 991.48 | 991.40 |
| 36 | 963.49 | 963.45 | 945.48 | 945.60 |
| 37 | 910.37 | 909.75 | 892.35 | 892.40 |
| 38 | 922.42 | 922.50 | 904.41 | 904.40 |
| 39 | 936.44 | 935.85 | 918.43 | 918.40 |
| 40 | 1003.44 | 1003.35 | 985.43 | 985.40 |
| 41 | 884.45 | 884.40 | 866.43 | 866.40 |
| 42 | 900.44 | 900.45 | 882.43 | 882.40 |
| 43 | 942.38 | 942.45 | 924.36 | 924.40 |
| 44 | 914.46 | 914.40 | 896.45 | 896.60 |
| 45 | 956.39 | 955.80 | 938.38 | 938.40 |
| 46 | 884.45 | 884.40 | 866.43 | 866.40 |
| 47 | 854.43 | 854.40 | 836.42 | 836.60 |
| 48 | 898.46 | 898.50 | 880.45 | 880.60 |
| 49 | 1003.52 | 1003.50 | 985.51 | 985.60 |
| 50 | 1033.53 | 1032.90 | 1015.52 | 1015.40 |
| 51 | 854.43 | 854.40 | 836.42 | 836.40 |
| 52 | 898.46 | 898.50 | 880.45 | 880.60 |
| 53 | 914.52 | 913.95 | 896.50 | 896.60 |
| 54 | 942.34 | 941.70 | 924.33 | 924.40 |
| 55 | 916.45 | 916.50 | 898.44 | 898.40 |
| 56 | 934.42 | 934.35 | 916.41 | 916.40 |
| 57 | 948.44 | 948.55 | 930.43 | 930.40 |
| 58 | 972.52 | 972.45 | 954.51 | 954.60 |
| 59 | 1077.55 | 1076.70 | 1059.54 | 1059.60 |
| 60 | 1063.54 | 1063.60 | 1045.53 | 1045.61 |
| 61 | 989.46 | 988.80 | 971.45 | 971.40 |
| 62 | 922.42 | 922.35 | 904.41 | 904.40 |
| 63 | 922.42 | 921.75 | 904.41 | 904.40 |
| 64 | 940.40 | 940.35 | 922.39 | 922.40 |
| 65 | 970.41 | 969.75 | 952.40 | 952.40 |
| 66 | 994.44 | 993.75 | 976.43 | 976.40 |
| 67 | 922.42 | 922.50 | 904.41 | 904.40 |
| 68 | 922.42 | 922.50 | 904.41 | 904.40 |
| 69 | 948.44 | 948.45 | 930.43 | 930.40 |
| 70 | 962.45 | 961.80 | 944.44 | 944.60 |
| 71 | 894.39 | 893.70 | 876.38 | 876.40 |
| 72 | 920.41 | 919.80 | 902.40 | 902.40 |
| 73 | 920.41 | 919.80 | 902.40 | 902.40 |
| 74 | 906.39 | 906.45 | 888.38 | 888.40 |
| 75 | 950.42 | 949.65 | 932.41 | 932.40 |
| 76 | 934.42 | 933.75 | 916.41 | 915.90 |
| 77 | 948.44 | 947.70 | 930.43 | 930.40 |
| 78 | 934.42 | 933.75 | 916.41 | 916.40 |
| 79 | 890.44 | 890.40 | 872.43 | 872.60 |
| 80 | 964.43 | 963.75 | 946.42 | 946.40 |
| 81 | 944.49 | 943.80 | 926.48 | 926.60 |
| 82 | 948.44 | 947.85 | 930.43 | 930.49 |
| 83 | 958.39 | 957.75 | 940.38 | 940.40 |
| 84 | 894.47 | 893.85 | 876.46 | 876.60 |
| 85 | 920.41 | 919.80 | 902.40 | 902.40 |
| 86 | 934.42 | 933.75 | 916.41 | 916.40 |
| 87 | 960.44 | 959.70 | 942.43 | 942.40 |
| 88 | 908.41 | 907.80 | 890.40 | 890.40 |
| 89 | 950.42 | 949.80 | 932.41 | 932.49 |
| 90 | 914.36 | 914.40 | 896.35 | 896.42 |
| 91 | 920.41 | 919.80 | 902.40 | 902.40 |
| 92 | 932.41 | 931.80 | 914.40 | 914.40 |
| 93 | 934.42 | 933.75 | 916.41 | 916.40 |
| 94 | 946.42 | 945.75 | 928.41 | 928.60 |
| 95 | 988.47 | 987.90 | 970.46 | 970.40 |
| 96 | 974.45 | 973.80 | 956.44 | 956.40 |
| 97 | 948.44 | 947.70 | 930.43 | 930.40 |
| 98 | 938.40 | 937.80 | 920.39 | 920.40 |
| 99 | 962.45 | 961.80 | 944.44 | 944.40 |
| 100 | 948.44 | 947.70 | 930.43 | 930.40 |
| 101 | 1103.57 | 1103.57 | 1085.56 | 1085.62 |
| 102 | 1061.52 | 1060.80 | 1043.51 | 1043.60 |
| 103 | 1007.51 | 1006.80 | 989.50 | 989.60 |
| 104 | 978.45 | 977.70 | 960.44 | 960.40 |
| 105 | 992.46 | 991.80 | 974.45 | 974.40 |
| 106 | 958.50 | 957.75 | 940.49 | 940.60 |
| 107 | 950.45 | 949.80 | 932.44 | 932.40 |
| 108 | 962.45 | 962.50 | 944.44 | 944.51 |
| 109 | 976.47 | 975.75 | 958.46 | 958.40 |
| 110 | 936.44 | 935.85 | 918.43 | 918.40 |
| 111 | 919.53 | 918.90 | 901.52 | 901.52 |
| 112 | 942.37 | 941.85 | 924.36 | 924.40 |
| 113 | 932.35 | 931.80 | 914.34 | 914.40 |
| 114 | 970.40 | 970.35 | 952.39 | 952.40 |
| 115 | 936.44 | 935.85 | 918.43 | 918.40 |
| 116 | 984.42 | 983.70 | 966.41 | 966.40 |
| 117 | 946.37 | 945.75 | 928.36 | 928.40 |
| 118 | 1044.44 | 1043.70 | 1026.43 | 1026.40 |
| 119 | 984.42 | 984.30 | 966.41 | 966.40 |
| 120 | 946.37 | 946.35 | 928.36 | 927.80 |
| 121 | 922.42 | 921.75 | 904.41 | 904.40 |
| 122 | 1075.50 | 1074.75 | 1057.49 | 1057.40 |
| 123 | 948.44 | 947.70 | 930.43 | 930.40 |
| 124 | 962.45 | 961.80 | 944.44 | 944.40 |
| 125 | 908.41 | 907.80 | 890.40 | 890.40 |
| 126 | 934.42 | 933.90 | 916.41 | 916.40 |
| 127 | 1049.52 | 1049.49 | 1031.51 | 1031.52 |
| 128 | 1010.43 | 1009.80 | 992.42 | 992.40 |
| 129 | 970.40 | 969.75 | 952.39 | 952.40 |
| 130 | 898.39 | 897.75 | 880.38 | 880.40 |
| 131 | 912.41 | 912.45 | 894.40 | 894.40 |
| 132 | 978.48 | 977.70 | 960.47 | 960.60 |
| 133 | 1111.51 | 1110.75 | 1093.50 | 1093.40 |
| 134 | 1147.49 | 1147.35 | 1129.48 | 1128.80 |
| 135 | 1061.46 | 1060.65 | 1043.45 | 1043.40 |
| 136 | 1047.44 | 1046.70 | 1029.43 | 1029.40 |
| 137 | 934.42 | 933.80 | 916.41 | 916.40 |
| 138 | 948.44 | 947.85 | 930.43 | 930.40 |
| 139 | 896.37 | 895.80 | 878.36 | 878.40 |
| 140 | 956.39 | 955.80 | 938.38 | 938.40 |
| 141 | 952.40 | 951.75 | 934.39 | 934.40 |
| 142 | 988.40 | 987.75 | 970.39 | 970.40 |
| 143 | 958.39 | 957.75 | 940.38 | 940.40 |
| 144 | 986.42 | 985.80 | 968.41 | 968.40 |
| 145 | 968.43 | 967.80 | 950.42 | 950.40 |
| 146 | 1010.44 | 1009.80 | 992.43 | 992.40 |
| 147 | 1010.44 | 1009.80 | 992.43 | 992.40 |
| 148 | 950.42 | 950.40 | 932.41 | 932.40 |
| 149 | 950.42 | 949.80 | 932.41 | 932.40 |
| 150 | 966.43 | 965.85 | 948.42 | 948.40 |
| 151 | 954.39 | 953.70 | 936.38 | 936.40 |
| 152 | 940.38 | 939.75 | 922.37 | 922.40 |
| 153 | 938.40 | 937.65 | 920.39 | 920.40 |
| 154 | 924.38 | 924.44 | 906.37 | 906.40 |
| 155 | 872.43 | 871.80 | 854.41 | 854.40 |
| 156 | 964.41 | 964.45 | 946.40 | 946.42 |
| 157 | 950.42 | 950.40 | 932.41 | 932.40 |
| 158 | 886.40 | 885.75 | 868.39 | 868.40 |
| 159 | 924.38 | 923.70 | 906.37 | 906.40 |
| 160 | 938.42 | 937.80 | 920.41 | 920.40 |
| 161 | 952.43 | 951.75 | 934.42 | 934.40 |
| 162 | 980.46 | 979.80 | 962.45 | 962.60 |
| 163 | 928.38 | 927.75 | 910.37 | 910.40 |
| 164 | 942.39 | 941.70 | 924.38 | 924.40 |
| 165 | 992.46 | 991.80 | 974.45 | 974.40 |
| 166 | 1010.45 | 1009.80 | 992.44 | 992.40 |
| 167 | 952.43 | 951.75 | 934.42 | 934.40 |
| 168 | 900.34 | 899.70 | 882.33 | 882.40 |
| 169 | 896.37 | 895.80 | 878.36 | 878.40 |
| 170 | 896.37 | 895.80 | 878.36 | 878.40 |
| 171 | 934.42 | 933.75 | 916.41 | 916.40 |
| 172 | 934.42 | 933.75 | 916.41 | 916.40 |
| 173 | 936.40 | 936.45 | 918.39 | 918.43 |
| 174 | 910.37 | 909.75 | 892.35 | 892.40 |
| 175 | 960.44 | 959.85 | 942.43 | 942.40 |
| 176 | 894.39 | 893.70 | 876.38 | 876.40 |
| 177 | 950.45 | 949.80 | 932.44 | 931.80 |
| 178 | 964.47 | 963.90 | 946.46 | 946.60 |
| 179 | 964.47 | 963.75 | 946.46 | 946.40 |
| 180 | 962.45 | 961.80 | 944.44 | 944.60 |
| 181 | 976.47 | 975.75 | 958.46 | 957.80 |
| 182 | 1007.51 | 1007.55 | 989.50 | 989.50 |
| 183 | 1049.52 | 1048.80 | 1031.51 | 1031.60 |
| 184 | 952.43 | 951.75 | 934.42 | 933.80 |
| 185 | 924.40 | 923.85 | 906.39 | 905.80 |
| 186 | 894.41 | 894.45 | 876.40 | 876.50 |
| 187 | 942.39 | 941.85 | 924.38 | 923.70 |
| 188 | 942.39 | 941.85 | 924.38 | 923.70 |
| 189 | 1004.34 | 1003.65 | 986.33 | 986.40 |
| 190 | 905.51 | 904.95 | 887.50 | 887.54 |
| 191 | 924.38 | 923.70 | 906.37 | 905.70 |
| 192 | 896.35 | 896.44 | 878.34 | 878.42 |
| 193 | 914.36 | 913.80 | 896.35 | 896.40 |
| 194 | 928.38 | 927.75 | 910.37 | 910.40 |
| 195 | 958.39 | 957.75 | 940.38 | 940.40 |
| 196 | 928.38 | 927.75 | 910.37 | 910.40 |
| 197 | 968.41 | 967.80 | 950.40 | 950.40 |
| 198 | 884.49 | 883.95 | 866.48 | 866.60 |
| 199 | 952.41 | 951.75 | 934.40 | 933.80 |
| 200 | 880.40 | 879.75 | 862.39 | 862.40 |
| 201 | 894.41 | 894.45 | 876.40 | 876.50 |
| 202 | 1057.49 | 1057.50 | 1039.48 | 1039.52 |
| 203 | 972.38 | 971.70 | 954.37 | 954.40 |
| 204 | 984.40 | 983.70 | 966.39 | 966.40 |
| 205 | 998.42 | 997.80 | 980.41 | 980.40 |
| 206 | 952.38 | 952.46 | 934.37 | 934.47 |
| 207 | 926.36 | 926.40 | 908.35 | 908.40 |
| 208 | 984.42 | 984.30 | 966.41 | 966.40 |
| 209 | 948.44 | 947.85 | 930.43 | 930.40 |
| 210 | 940.38 | 939.31 | 922.37 | 922.40 |
| 211 | 952.41 | 952.35 | 934.40 | 933.80 |
| 212 | 966.43 | 966.30 | 948.42 | 947.80 |
| 213 | 908.41 | 907.80 | 890.40 | 889.80 |
| 214 | 898.39 | 897.75 | 880.37 | 879.80 |
| 215 | 894.41 | 894.45 | 876.40 | 876.40 |
| 216 | 939.46 | 939.45 | 921.45 | 921.40 |
| 217 | 939.46 | 938.85 | 921.45 | 921.40 |
| 218 | 945.49 | 945.45 | 927.48 | 927.60 |
| 219 | 910.37 | 910.35 | 892.35 | 891.80 |
| 220 | 964.41 | 964.35 | 946.40 | 945.80 |
| 221 | 910.39 | 910.35 | 892.37 | 891.80 |
| 222 | 906.39 | 906.30 | 888.38 | 887.80 |
| 223 | 920.41 | 920.40 | 902.40 | 901.80 |
| 224 | 966.39 | 966.30 | 948.38 | 948.40 |
| 225 | 950.40 | 950.40 | 932.39 | 932.40 |
| 226 | 976.41 | 976.35 | 958.40 | 958.40 |
| 227 | 940.38 | 940.35 | 922.37 | 922.40 |
| 228 | 936.38 | 936.30 | 918.37 | 918.39 |
| 229 | 922.37 | 922.35 | 904.35 | 904.40 |
| 230 | 980.43 | 980.40 | 962.42 | 962.40 |
| 231 | 994.44 | 994.35 | 976.43 | 976.40 |
| 232 | 1020.46 | 1020.48 | 1002.45 | 1002.50 |
| 233 | 922.42 | 922.35 | 904.41 | 903.80 |
| 234 | 912.40 | 912.30 | 894.39 | 893.70 |
| 235 | 952.43 | 952.35 | 934.42 | 933.80 |
| 236 | 950.45 | 950.40 | 932.44 | 931.80 |
| 237 | 952.41 | 952.00 | 934.40 | 934.40 |
| 238 | 992.44 | 991.80 | 974.43 | 974.40 |
| 239 | 942.39 | 941.70 | 924.38 | 924.40 |
| 240 | 974.38 | 973.80 | 956.37 | 956.40 |
| 241 | 988.40 | 987.75 | 970.39 | 970.40 |
| 242 | 1016.43 | 1015.80 | 998.42 | 998.40 |
| 243 | 954.44 | 953.85 | 936.43 | 936.40 |
| 244 | 982.47 | 981.90 | 964.46 | 964.40 |
| 245 | 1038.45 | 1037.70 | 1020.44 | 1020.40 |
| 246 | 1024.43 | 1023.75 | 1006.42 | 1006.40 |
| 247 | 1052.46 | 1051.80 | 1034.45 | 1034.40 |
| 248 | 944.37 | 943.80 | 926.36 | 926.40 |
| 249 | 940.40 | 939.75 | 922.39 | 922.40 |
| 250 | 958.39 | 957.75 | 940.38 | 940.40 |
| 251 | 942.39 | 941.70 | 924.38 | 924.40 |
| 252 | 928.38 | 927.75 | 910.37 | 910.40 |
| 253 | 941.49 | 940.95 | 923.48 | 922.80 |
| 254 | 953.49 | 952.80 | 935.48 | 935.60 |
| 255 | 900.42 | 899.85 | 882.41 | 882.40 |
| 256 | 942.39 | 942.45 | 924.38 | 924.42 |
| 257 | 966.43 | 965.70 | 948.42 | 948.40 |
| 258 | 938.40 | 937.80 | 920.39 | 920.40 |
| 259 | 980.41 | 979.80 | 962.40 | 962.40 |
| 260 | 924.38 | 923.85 | 906.37 | 906.40 |
| 261 | 938.40 | 938.44 | 920.39 | 920.50 |
| 262 | 928.38 | 927.75 | 910.37 | 910.40 |
| 263 | 926.36 | 925.80 | 908.35 | 908.40 |
| 264 | 1002.30 | 1001.70 | 984.29 | 984.40 |
| 265 | 984.31 | 984.30 | 966.30 | 966.30 |
| 266 | 1006.30 | 1006.35 | 988.29 | 988.20 |
| 267 | 914.52 | 913.95 | 896.50 | 896.60 |
| 268 | 924.38 | 923.85 | 906.37 | 906.40 |
| 269 | 910.37 | 909.75 | 892.35 | 892.40 |
| 270 | 910.37 | 910.36 | 892.35 | 892.40 |
| 271 | 966.39 | 965.70 | 948.38 | 948.40 |
| 272 | 970.39 | 969.75 | 952.38 | 952.40 |
| 273 | 970.39 | 969.75 | 952.38 | 952.40 |
| 274 | 970.39 | 969.75 | 952.38 | 952.40 |
| 275 | 940.38 | 939.75 | 922.37 | 922.40 |
| 276 | 966.39 | 966.45 | 948.38 | 948.40 |
| 277 | 970.37 | 969.75 | 952.36 | 951.70 |
| 278 | 990.35 | 989.70 | 972.34 | 971.70 |
| 279 | 894.41 | 893.85 | 876.40 | 876.40 |
| 280 | 908.43 | 907.80 | 890.42 | 890.40 |
| 281 | 906.41 | 905.70 | 888.40 | 888.40 |
| 282 | 894.41 | 893.85 | 876.40 | 876.40 |
| 283 | 952.40 | 951.75 | 934.39 | 934.40 |
| 284 | 966.41 | 965.70 | 948.40 | 948.40 |
| 285 | 1053.48 | 1052.70 | 1035.47 | 1035.40 |
| 286 | 952.40 | 951.80 | 934.39 | 934.40 |
| 287 | 908.37 | 907.80 | 890.36 | 890.40 |
| 288 | 926.36 | 926.41 | 908.35 | 908.42 |
| 289 | 954.37 | 953.70 | 936.36 | 936.40 |
| 290 | 940.36 | 939.75 | 922.35 | 921.80 |
| 291 | 990.43 | 990.48 | 972.42 | 972.43 |
| 292 | 936.38 | 935.70 | 918.37 | 918.40 |
| 293 | 940.38 | 940.42 | 922.37 | 922.41 |
| 294 | 954.39 | 954.30 | 936.38 | 936.42 |
| 295 | 984.40 | 983.70 | 966.39 | 966.40 |
| 296 | 896.35 | 895.80 | 878.34 | 878.40 |
| 297 | 910.37 | 909.75 | 892.35 | 892.40 |
| 298 | 910.37 | 910.35 | 892.35 | 892.40 |
| 299 | 924.38 | 923.70 | 906.37 | 906.40 |
| 300 | 924.38 | 923.85 | 906.37 | 906.40 |
| 301 | 938.40 | 937.80 | 920.39 | 920.40 |
| 302 | 924.38 | 923.85 | 906.37 | 906.40 |
| 303 | 938.40 | 937.80 | 920.39 | 920.40 |
| 304 | 900.34 | 899.70 | 882.33 | 882.40 |
| 305 | 959.39 | 959.40 | 941.38 | 941.40 |
| 306 | 959.39 | 959.49 | 941.38 | 941.47 |
| 307 | 956.44 | 956.53 | 938.43 | 938.40 |
| 308 | 1038.39 | 1037.70 | 1020.38 | 1020.30 |
| 309 | 1048.44 | 1047.75 | 1030.42 | 1030.40 |
| 310 | 1054.36 | 1054.43 | 1036.35 | 1036.43 |
| 311 | 1072.47 | 1071.75 | 1054.46 | 1054.40 |
| 312 | 1078.40 | 1077.60 | 1060.39 | 1060.40 |
| 313 | 924.43 | 923.85 | 906.41 | 906.40 |
| 314 | 1008.44 | 1008.50 | 990.43 | 990.50 |
| 315 | 1020.44 | 1019.70 | 1002.43 | 1002.40 |
| 316 | 954.37 | 954.30 | 936.36 | 936.39 |
| 317 | 972.36 | 972.37 | 954.35 | 954.43 |
| 318 | 960.39 | 960.45 | 942.38 | 942.40 |
| 319 | 952.41 | 951.75 | 934.40 | 934.40 |
| 320 | 950.40 | 949.80 | 932.39 | 932.40 |
| 321 | 976.41 | 975.75 | 958.40 | 958.40 |
| 322 | 922.45 | 921.90 | 904.44 | 904.40 |
| 323 | 962.34 | 961.65 | 944.33 | 944.30 |
| 324 | 912.34 | 911.70 | 894.33 | 894.40 |
| 325 | 938.40 | 937.80 | 920.39 | 920.40 |
| 326 | 1008.42 | 1007.70 | 990.41 | 990.40 |
| 327 | 1019.44 | 1018.65 | 1001.43 | 1001.40 |
| 328 | 1031.44 | 1030.65 | 1013.43 | 1013.40 |
| 329 | 942.37 | 942.30 | 924.36 | 924.30 |
| 330 | 986.38 | 986.40 | 968.37 | 968.48 |
| 331 | 950.40 | 950.50 | 932.39 | 932.43 |
| 332 | 954.39 | 954.46 | 936.38 | 936.49 |
| 333 | 954.39 | 953.70 | 936.38 | 936.30 |
| 334 | 968.41 | 967.80 | 950.40 | 949.70 |
| 335 | 938.36 | 938.41 | 920.35 | 920.47 |
| 336 | 952.38 | 952.39 | 934.37 | 934.46 |
| 337 | 952.38 | 951.75 | 934.37 | 934.40 |
| 338 | 966.39 | 966.42 | 948.38 | 948.43 |
| 339 | 966.37 | 966.43 | 948.36 | 948.42 |
| 340 | 980.39 | 980.25 | 962.38 | 961.70 |
| 341 | 980.39 | 979.65 | 962.38 | 962.40 |
| 342 | 990.35 | 990.26 | 972.34 | 972.35 |
| 343 | 980.39 | 980.40 | 962.38 | 961.70 |
| 344 | 1016.37 | 1016.40 | 998.36 | 998.46 |
| 345 | 922.37 | 922.35 | 904.35 | 903.80 |
| 346 | 934.37 | 934.35 | 916.35 | 915.80 |
| 347 | 936.38 | 936.49 | 918.37 | 918.46 |
| 348 | 970.37 | 970.37 | 952.36 | 952.48 |
| 349 | 918.39 | 918.30 | 900.38 | 900.40 |
| 350 | 984.38 | 983.70 | 966.37 | 966.40 |
| 351 | 922.39 | 922.35 | 904.37 | 904.40 |
| 352 | 936.40 | 936.45 | 918.39 | 918.40 |
| 353 | 976.41 | 976.51 | 958.40 | 958.41 |
| 354 | 912.34 | 911.70 | 894.33 | 894.30 |
| 355 | 894.41 | 894.50 | 876.40 | 876.53 |
| 356 | 980.33 | 980.25 | 962.32 | 962.20 |
| 357 | 998.32 | 998.25 | 980.31 | 980.20 |
| 358 | 924.41 | 924.45 | 906.40 | 906.40 |
| 359 | 984.33 | 984.30 | 966.31 | 966.40 |
| 360 | 1002.32 | 1002.25 | 984.31 | 984.30 |
| 361 | 924.41 | 924.45 | 906.40 | 906.49 |
| 362 | 924.41 | 924.52 | 906.40 | 906.50 |
| 363 | 880.38 | 880.35 | 862.37 | 862.40 |
| 364 | 910.39 | 910.35 | 892.38 | 892.40 |
| 365 | 910.39 | 910.35 | 892.38 | 892.40 |
| 366 | 898.39 | 898.35 | 880.38 | 880.40 |
| 367 | 990.37 | 990.38 | 972.36 | 972.40 |
| 368 | 964.36 | 964.35 | 946.35 | 946.38 |
| 369 | 988.36 | 988.35 | 970.35 | 970.39 |
| 370 | 962.34 | 962.40 | 944.33 | 944.37 |
| 371 | 876.40 | 876.45 | 858.39 | 858.40 |
| 372 | 972.38 | 972.45 | 954.37 | 954.30 |
| 373 | 954.39 | 954.30 | 936.38 | 936.30 |
| 374 | 1040.35 | 1040.25 | 1022.34 | 1022.30 |
| 375 | 1068.38 | 1068.30 | 1050.37 | 1050.40 |
| 376 | 1038.39 | 1038.45 | 1020.38 | 1020.30 |
| 377 | 1051.42 | 1051.35 | 1033.41 | 1033.40 |
| 378 | 1062.42 | 1062.30 | 1044.41 | 1044.30 |
| 379 | 1020.36 | 1020.30 | 1002.35 | 1002.40 |
| 380 | 954.40 | 954.44 | 936.39 | 936.43 |
| 381 | 1000.40 | 1000.41 | 982.39 | 982.46 |
| 382 | 980.39 | 980.42 | 962.38 | 962.50 |
| 383 | 1058.46 | 1058.40 | 1040.45 | 1040.40 |
| 384 | 1050.37 | 1050.30 | 1032.36 | 1032.30 |
| 385 | 1064.39 | 1064.40 | 1046.37 | 1046.40 |
| 386 | 1092.42 | 1092.30 | 1074.41 | 1074.40 |
| 387 | 1058.46 | 1058.40 | 1040.45 | 1040.40 |
| 388 | 1106.43 | 1106.40 | 1088.42 | 1088.30 |
| 389 | 906.41 | 906.52 | 888.40 | 888.52 |
| 390 | 920.43 | 920.42 | 902.42 | 902.50 |
| 391 | 954.39 | 954.44 | 936.38 | 936.47 |
| 392 | 932.47 | 932.55 | 914.46 | 914.40 |
| 393 | 952.41 | 952.35 | 934.40 | 934.40 |
| 394 | 908.45 | 908.40 | 890.44 | 890.40 |
| 395 | 948.48 | 948.45 | 930.47 | 930.50 |
| 396 | 948.44 | 948.45 | 930.43 | 930.50 |
| 397 | 1047.51 | 1047.45 | 1029.50 | 1029.50 |
| 398 | 1054.36 | 1054.35 | 1036.35 | 1036.40 |
| 399 | 1038.39 | 1038.30 | 1020.38 | 1020.40 |
| 400 | 890.44 | 890.59 | 872.43 | 872.57 |
| 401 | 954.40 | 954.52 | 936.39 | 936.45 |
| 402 | 1000.40 | 1000.50 | 982.39 | 982.47 |
| 403 | 982.41 | 982.41 | 964.40 | 964.34 |
| 404 | 940.38 | 940.47 | 922.37 | 922.40 |
| 405 | 1025.43 | 1025.53 | 907.37 | 907.49 |
| 406 | 1025.43 | 1025.49 | 907.37 | 907.50 |
| 407 | 924.38 | 924.48 | 906.37 | 906.48 |
| 408 | 940.38 | 940.35 | 922.37 | 922.41 |
| 409 | 912.34 | 912.00 | 894.33 | 894.45 |
| 410 | 926.36 | 926.46 | 908.35 | 908.40 |
| 411 | 926.36 | 926.44 | 908.35 | 908.40 |
| 412 | 912.41 | 912.52 | 894.40 | 894.50 |
| 413 | 922.39 | 922.52 | 904.38 | 904.46 |
| 414 | 990.43 | 990.45 | 972.42 | 972.50 |
| 415 | 960.44 | 960.50 | 942.43 | 942.52 |
| 416 | 1000.42 | 1000.51 | 982.41 | 982.50 |
| 417 | 1016.39 | 1016.50 | 998.38 | 998.49 |
| 418 | 984.45 | 984.56 | 966.44 | 966.53 |
| 419 | 972.44 | 972.50 | 954.43 | 954.53 |
| 420 | 986.46 | 986.57 | 968.45 | 968.53 |
| 421 | 1004.45 | 1004.58 | 986.44 | 986.59 |
| 422 | 952.38 | 952.41 | 934.37 | 934.50 |
| 423 | 999.41 | 999.50 | 881.35 | 881.50 |
| 424 | 900.34 | 900.42 | 882.33 | 882.38 |
| 425 | 995.49 | 995.52 | 877.38 | 877.46 |
| 426 | 1028.43 | 1028.54 | 1010.42 | 1010.49 |
| 427 | 918.43 | 918.00 | 900.42 | 900.52 |
| 428 | 972.40 | 972.43 | 954.39 | 954.47 |
| 429 | 960.44 | 960.46 | 942.43 | 942.50 |
| 430 | 982.42 | 982.51 | 964.41 | 964.44 |
| 431 | 1004.37 | 1004.38 | 986.36 | 986.33 |
| 432 | 1109.50 | 1109.60 | 1091.49 | 1091.49 |
| 433 | 1095.49 | 1095.60 | 1077.48 | 1077.52 |
| 434 | 1091.51 | 1091.60 | 1073.50 | 1073.55 |
| 435 | 1077.50 | 1077.60 | 1059.49 | 1059.54 |
| 436 | 1018.38 | 1018.37 | 1000.37 | 1000.39 |
| 437 | 966.39 | 966.87 | 948.38 | 948.44 |
| 438 | 940.38 | 940.46 | 922.37 | 922.53 |
| 439 | 934.45 | 934.57 | 916.44 | 916.55 |
| 440 | 980.41 | 980.00 | 962.40 | 962.47 |
| 441 | 966.39 | 966.43 | 948.38 | 948.49 |
| 442 | 1014.41 | 1014.46 | 996.40 | 996.40 |
| 443 | 984.40 | 984.00 | 966.39 | 966.48 |
| 444 | 1000.40 | 1000.50 | 982.39 | 982.43 |
| 445 | 1056.40 | 1056.44 | 1038.39 | 1038.48 |
| 446 | 1026.41 | 1026.46 | 1008.40 | 1008.54 |
| 447 | 1012.39 | 1012.44 | 994.38 | 994.40 |
| 448 | 994.40 | 994.41 | 976.39 | 976.41 |
| 449 | 978.43 | 978.48 | 960.42 | 960.42 |
| 450 | 992.44 | 992.47 | 974.43 | 974.41 |
| 451 | 934.45 | 934.47 | 916.44 | 916.45 |
| 452 | 948.46 | 948.48 | 930.45 | 930.56 |
| 453 | 968.41 | 968.48 | 950.40 | 950.46 |
| 454 | 950.40 | 950.40 | 932.39 | 932.42 |
| 455 | 980.41 | 980.50 | 962.40 | 962.49 |
| 456 | 994.42 | 994.44 | 976.41 | 976.43 |
| 457 | 996.42 | 996.00 | 978.41 | 978.85 |
| 458 | 1012.39 | 1012.32 | 994.38 | 995.10 |
| 459 | 954.39 | 954.48 | 936.38 | 936.40 |
| 460 | 988.39 | 988.47 | 970.38 | 970.47 |
| 461 | 925.42 | 925.55 | 907.40 | 907.60 |
| 462 | 1023.41 | 1023.46 | 1005.40 | 1005.60 |
| 463 | 983.39 | 983.49 | 965.38 | 965.50 |
| 464 | 987.38 | 987.44 | 969.37 | 969.49 |
| 465 | 1033.38 | 1033.40 | 1015.37 | 1015.49 |
| 466 | 1073.42 | 1073.47 | 1055.40 | 1055.81 |
| 467 | 954.39 | 954.48 | 936.38 | 936.44 |
| 468 | 894.41 | 894.54 | 876.40 | 876.53 |
| 469 | 910.37 | 910.47 | 892.35 | 892.48 |
| 470 | 938.36 | 938.40 | 920.35 | 920.40 |
| 471 | 938.36 | 938.47 | 920.35 | 920.41 |
| 472 | 958.37 | 958.44 | 940.36 | 940.47 |
| 473 | 968.41 | 968.49 | 950.40 | 950.49 |
| 474 | 1000.39 | 1000.49 | 982.38 | 982.45 |
| 475 | 966.39 | 966.51 | 948.38 | 948.39 |
| 476 | 978.39 | 978.50 | 960.38 | 960.48 |
| 477 | 946.45 | 946.55 | 928.44 | 928.54 |
| 478 | 990.40 | 990.46 | 972.39 | 972.48 |
| 479 | 984.36 | 984.43 | 966.35 | 966.43 |
| 480 | 968.39 | 968.00 | 950.38 | 950.43 |
| 481 | 968.39 | 968.48 | 950.38 | 950.43 |
| 482 | 972.41 | 972.51 | 954.40 | 954.48 |
| 483 | 1075.46 | 1075.00 | 1057.45 | 1057.58 |
| 484 | 966.43 | 966.51 | 948.42 | 948.55 |
| 485 | 1000.39 | 1000.47 | 982.38 | 982.44 |
| 486 | 960.36 | 960.46 | 942.35 | 942.50 |
| 487 | 970.41 | 970.51 | 952.40 | 952.48 |
| 488 | 1044.42 | 1044.46 | 970.35 | 970.42 |
| 489 | 986.38 | 986.43 | 968.37 | 968.48 |
| 490 | 958.35 | 958.44 | 940.34 | 940.42 |
| 491 | 938.36 | 938.44 | 920.35 | 920.48 |
| 492 | 922.37 | 922.43 | 904.35 | 904.49 |
| 493 | 1053.46 | 1053.51 | 1035.45 | 1035.56 |
| 494 | 904.40 | 904.53 | 886.39 | 886.50 |
| 495 | 918.41 | 918.55 | 900.40 | 900.52 |
| 496 | 920.43 | 920.55 | 902.42 | 902.55 |
| 497 | 986.38 | 986.48 | 968.37 | 968.43 |
| 498 | 1074.43 | 1074.00 | 1056.42 | 1056.51 |
| 499 | 1056.42 | 1056.00 | 1038.41 | 1038.48 |
| 500 | 1044.40 | 1044.90 | 1026.39 | 1026.44 |
| 501 | 1014.41 | 1014.49 | 996.40 | 996.48 |
| 502 | 1026.41 | 1026.36 | 1008.40 | 1008.41 |
| 503 | 956.44 | 956.00 | 938.43 | 938.52 |
| 504 | 943.42 | 944.00 | 925.41 | 925.46 |
| 505 | 1061.42 | 1062.00 | 1043.40 | 1043.47 |
| 506 | 1105.50 | 1106.00 | 1087.49 | 1087.62 |
| 507 | 1047.40 | 1048.00 | 1029.39 | 1029.38 |
| 508 | 1051.45 | 1051.00 | 1033.44 | 1033.50 |
| 509 | 953.43 | 953.00 | 935.42 | 935.53 |
| 510 | 959.39 | 959.00 | 941.38 | 941.49 |
| 511 | 990.45 | 990.00 | 972.44 | 972.52 |
| 512 | 1000.51 | 1000.00 | 982.50 | 982.57 |
| 513 | 1069.48 | 1069.00 | 1051.47 | 1051.57 |
| 514 | 990.45 | 991.00 | 972.44 | 972.51 |
| 515 | 970.48 | 971.00 | 952.47 | 952.40 |
| 516 | 964.42 | 964.50 | 946.41 | 946.45 |
| 517 | 962.48 | 962.56 | 944.47 | 944.58 |
| 518 | 1014.41 | 1014.00 | 996.40 | 996.48 |
| 519 | 1050.45 | 1050.00 | 1032.44 | 1032.39 |
| 520 | 1010.42 | 1010.00 | 992.41 | 992.36 |
| 521 | 996.40 | 996.00 | 978.39 | 978.37 |
| 522 | 994.42 | 994.00 | 976.41 | 976.50 |
| 523 | 1002.41 | 1002.00 | 984.40 | 984.47 |
| 524 | 984.50 | 984.00 | 966.49 | 966.52 |
| 525 | 944.47 | 944.00 | 926.46 | 926.57 |
| 526 | 982.48 | 982.00 | 964.47 | 964.50 |
| 527 | 942.45 | 942.50 | 924.44 | 924.53 |
| 528 | 942.51 | 942.00 | 924.50 | 924.59 |
| 529 | 1011.48 | 1011.00 | 993.46 | 993.48 |
| 530 | 988.47 | 988.00 | 970.46 | 970.56 |
| 531 | 962.45 | 962.00 | 944.44 | 944.60 |
| 532 | 1053.47 | 1053.56 | 1035.46 | 1035.50 |
| 533 | 1039.45 | 1039.53 | 1021.44 | 1021.47 |
| 534 | 1040.45 | 1040.00 | 1022.44 | 1022.47 |
| 535 | 1049.42 | 1049.90 | 1031.40 | 1031.49 |
| 536 | 1077.45 | 1077.00 | 1059.44 | 1059.49 |
| 537 | 1035.40 | 1035.00 | 1017.39 | 1017.46 |
| 538 | 1063.43 | 1063.00 | 1045.42 | 1045.50 |
| 539 | 980.45 | 980.00 | 962.44 | 962.45 |
| 540 | 1036.40 | 1036.00 | 1018.39 | 1018.32 |
| 541 | 934.45 | 934.00 | 916.44 | 916.44 |
| 542 | 962.48 | 963.00 | 944.47 | 944.46 |
| 543 | 1003.41 | 1003.49 | 985.40 | 985.47 |
| 544 | 1031.44 | 1031.80 | 1013.43 | 1013.52 |
| 545 | 964.41 | 965.00 | 946.40 | 946.49 |
| 546 | 996.40 | 996.00 | 978.39 | 978.46 |
| 547 | 970.39 | 970.49 | 952.37 | 952.46 |
| 548 | 1016.39 | 1016.00 | 998.38 | 998.45 |
| 549 | 1018.50 | 1018.00 | 1000.49 | 1000.58 |
| 550 | 1004.44 | 1004.00 | 986.43 | 986.53 |
| 551 | 1018.46 | 1018.00 | 1000.45 | 1000.51 |
| 552 | 984.38 | 984.40 | 966.37 | 966.45 |
| 553 | 938.38 | 938.00 | 920.37 | 920.44 |
| 554 | 932.47 | 932.00 | 914.46 | 914.55 |
| 555 | 914.48 | 914.00 | 896.47 | 896.60 |
| 556 | 946.48 | 946.00 | 928.47 | 928.56 |
| 557 | 952.44 | 952.00 | 934.43 | 934.54 |
| 558 | 934.45 | 934.00 | 916.44 | 916.51 |
| 559 | 956.39 | 956.00 | 938.38 | 938.44 |
| 560 | 934.42 | 935.00 | 916.41 | 916.90 |
| 561 | 964.43 | 965.00 | 946.42 | 946.37 |
| 562 | 882.33 | 882.00 | 864.32 | 864.31 |
| 563 | 1026.41 | 1026.00 | 1008.40 | 1008.33 |
| 564 | 972.44 | 972.00 | 954.43 | 954.40 |
| 565 | 958.42 | 958.00 | 940.41 | 940.41 |
| 566 | 1040.42 | 1040.00 | 1022.41 | 1023.02 |
| 567 | 1054.44 | 1054.40 | 1036.43 | 1036.40 |
| 568 | 1063.43 | 1063.36 | 1045.42 | 1045.40 |
| 569 | 1014.41 | 1014.00 | 996.40 | 996.39 |
| 570 | 1000.39 | 1000.00 | 982.38 | 982.38 |
| 571 | 1064.46 | 1064.00 | 1046.45 | 1046.43 |
| 572 | 990.43 | 990.90 | 972.42 | 972.50 |
| 573 | 980.41 | 980.00 | 962.40 | 962.40 |
| 574 | 1028.42 | 1028.00 | 1010.41 | 1010.40 |
| 575 | 992.44 | 992.41 | 974.43 | 974.36 |
| 576 | 1040.42 | 1040.00 | 1022.41 | 1022.40 |
| 577 | 986.42 | 986.00 | 968.41 | 968.49 |
| 578 | 1028.42 | 1028.50 | 1010.41 | 1010.50 |
| 579 | 980.41 | 980.00 | 962.40 | 962.42 |
| 580 | 964.41 | 964.00 | 946.40 | 946.89 |
| 581 | 1022.43 | 1022.00 | 1004.42 | 1004.50 |
| 582 | 1099.47 | 1099.00 | 1081.46 | 1081.52 |
| 583 | 1087.47 | 1087.00 | 1069.46 | 1069.50 |
| 584 | 1030.46 | 1030.00 | 1012.45 | 1012.51 |
| 585 | 1075.43 | 1075.00 | 1057.42 | 1057.41 |
| 586 | 1079.48 | 1079.43 | 1061.47 | 1061.43 |
| 587 | 998.38 | 998.34 | 980.37 | 980.42 |
| 588 | 982.40 | 982.00 | 964.39 | 964.45 |
| 589 | 1174.54 | 1174.00 | 1156.53 | 1156.51 |
| 590 | 1023.48 | 1023.41 | 1005.47 | 1005.44 |
| 591 | 1039.45 | 1039.95 | 1021.44 | 1021.43 |
| 592 | 1067.48 | 1068.01 | 1049.47 | 1049.46 |
| 593 | 1053.47 | 1054.00 | 1035.46 | 1035.51 |
| 594 | 1053.47 | 1053.60 | 1035.46 | 1035.41 |
| 595 | 1040.45 | 1040.40 | 1022.43 | 1022.47 |
| 596 | 1036.50 | 1036.00 | 1018.49 | 1018.52 |
| 597 | 1036.50 | 1036.00 | 1018.49 | 1018.57 |
| 598 | 1037.50 | 1038.00 | 1019.49 | 1019.49 |
| 599 | 1046.51 | 1046.00 | 972.44 | 972.37 |
| 600 | 1018.48 | 1018.00 | 944.41 | 944.46 |
| 601 | 1046.51 | 1046.00 | 972.44 | 972.50 |
| 602 | 1000.39 | 1000.35 | 982.38 | 982.41 |
| 603 | 1072.45 | 1072.45 | 998.38 | 998.48 |
| 604 | 980.41 | 980.00 | 962.40 | 962.39 |
| 605 | 1029.43 | 1029.00 | 1011.42 | 1011.36 |
| 606 | 968.41 | 968.39 | 950.40 | 950.43 |
| 607 | 1017.43 | 1017.00 | 999.42 | 999.38 |
| 608 | 1121.47 | 1121.44 | 1047.40 | 1047.37 |
| 609 | 1075.47 | 1075.45 | 1001.40 | 1001.42 |
| 610 | 1024.43 | 1024.00 | 1006.42 | 1006.48 |
| 611 | 1123.45 | 1123.00 | 1105.44 | 1105.41 |
| 612 | 1093.44 | 1093.42 | 1019.37 | 1019.35 |
| 613 | 1086.47 | 1086.44 | 1012.39 | 1012.45 |
| 614 | 1107.46 | 1107.57 | 1033.38 | 1033.42 |
| 615 | 1129.51 | 1130.07 | 1111.50 | 1111.48 |
| 616 | 1136.48 | 1136.60 | 1118.47 | 1118.53 |
| 617 | 966.43 | 966.00 | 948.42 | 948.49 |
| 618 | 987.42 | 987.00 | 969.41 | 969.53 |
| 619 | 1037.43 | 1037.00 | 1019.42 | 1019.49 |
| 620 | 1047.40 | 1047.36 | 1029.39 | 1029.49 |
| 621 | 1019.44 | 1019.00 | 1001.43 | 1001.56 |
| 622 | 964.41 | 964.00 | 946.40 | 946.45 |
| 623 | 982.45 | 982.00 | 964.44 | 964.50 |
| 624 | 1033.49 | 1033.60 | 1015.48 | 1015.55 |
| 625 | 968.41 | 968.00 | 950.40 | 950.46 |
| 626 | 1054.54 | 1055.00 | 1036.53 | 1036.48 |
| 627 | 1026.51 | 1026.57 | 1008.50 | 1008.55 |
| 628 | 1012.49 | 1013.00 | 994.48 | 994.44 |
| 629 | 1048.49 | 1048.46 | 1030.48 | 1030.46 |
| 630 | 1004.49 | 1004.51 | 986.48 | 986.45 |
| 631 | 962.44 | 962.43 | 944.43 | 944.41 |
| 632 | 998.44 | 998.45 | 980.43 | 980.39 |
| 633 | 986.45 | 986.54 | 968.44 | 968.50 |
| 634 | 1027.45 | 1027.00 | 1009.44 | 1009.45 |
| 635 | 966.39 | 966.37 | 948.38 | 948.42 |
| 636 | 994.42 | 994.35 | 976.41 | 976.46 |
| 637 | 1029.43 | 1029.80 | 1011.42 | 1011.52 |
| 638 | 1029.43 | 1029.50 | 1011.42 | 1011.47 |
| 639 | 1056.40 | 1056.00 | 1038.39 | 1038.48 |
| 640 | 1020.42 | 1020.00 | 1002.41 | 1002.51 |
| 641 | 955.43 | 956.00 | 937.42 | 937.52 |
| 642 | 906.41 | 906.41 | 888.40 | 888.49 |
| 643 | 956.43 | 956.00 | 938.42 | 938.48 |
| 644 | 907.40 | 907.00 | 889.39 | 889.47 |
| 645 | 996.46 | 996.00 | 978.45 | 978.51 |
| 646 | 947.44 | 947.00 | 929.43 | 929.54 |
| 647 | 997.42 | 997.00 | 979.41 | 979.52 |
| 648 | 1044.40 | 1044.00 | 1026.39 | 1026.48 |
| 649 | 1026.43 | 1026.00 | 1008.42 | 1008.51 |
| 650 | 958.35 | 958.32 | 940.34 | 940.41 |
| 651 | 1039.37 | 1040.00 | 1021.36 | 1021.43 |
| 652 | 1040.52 | 1040.60 | 1022.51 | 1022.57 |
| 653 | 1026.51 | 1027.00 | 1008.50 | 1008.56 |
| 654 | 1054.54 | 1054.00 | 1036.53 | 1036.71 |
| 655 | 1040.52 | 1040.00 | 1022.51 | 1022.61 |
| 656 | 1040.45 | 1040.50 | 1022.44 | 1022.51 |
| 657 | 1022.46 | 1022.53 | 1004.45 | 1004.55 |
| 658 | 1054.46 | 1054.00 | 1036.45 | 1036.48 |
| 659 | 974.46 | 974.00 | 956.45 | 956.59 |
| 660 | 1014.41 | 1014.00 | 996.40 | 996.49 |
| 661 | 1004.39 | 1004.00 | 986.38 | 986.42 |
| 662 | 1020.53 | 1020.60 | 1002.52 | 1002.56 |
| 663 | 1027.50 | 1027.60 | 1009.49 | 1009.60 |
| 664 | 1050.52 | 1050.60 | 1032.51 | 1032.58 |
| 665 | 1051.48 | 1051.00 | 1033.47 | 1033.49 |
| 666 | 1072.49 | 1072.00 | 1054.48 | 1054.50 |
| 667 | 1090.48 | 1090.00 | 1072.47 | 1072.57 |
| 668 | 1033.49 | 1033.00 | 1015.48 | 1015.55 |
| 669 | 999.51 | 999.00 | 981.50 | 981.59 |
| 670 | 1000.49 | 1000.00 | 982.48 | 982.51 |
| 671 | 1050.52 | 1050.00 | 1032.51 | 1032.55 |
| 672 | 1050.52 | 1050.00 | 1032.51 | 1032.52 |
| 673 | 1034.49 | 1034.00 | 1016.48 | 1016.51 |
| 674 | 1055.54 | 1055.00 | 1037.52 | 1037.56 |
| 675 | 1054.46 | 1054.00 | 1036.45 | 1036.53 |
| 676 | 1043.44 | 1043.50 | 1025.43 | 1025.48 |
| 677 | 1113.55 | 1113.70 | 1095.54 | 1095.61 |
| 678 | 1097.56 | 1097.70 | 1079.55 | 1079.62 |
| 679 | 1087.54 | 1087.70 | 1069.53 | 1069.62 |
| 680 | 1012.39 | 1012.32 | 994.38 | 995.5 |

Table 4, below, provides the full chemical structure for each exemplified compound in Table 2A and B.

TABLE 4
Chemical Structure for Exemplary
Compounds Described in Table 2A and B

| Ex. | Structure |
|---|---|
| 1 | 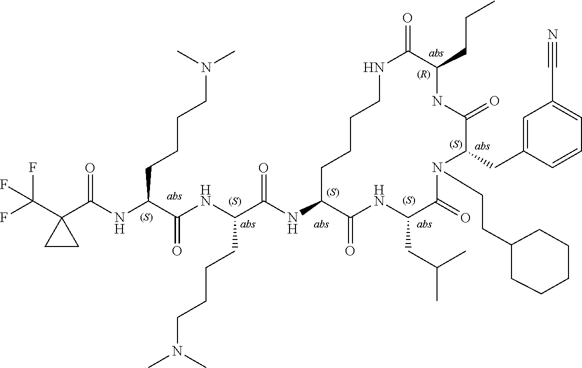 |
| 2 | 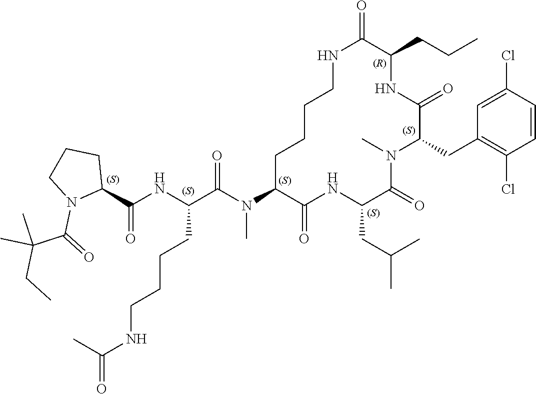 |
| 3 | 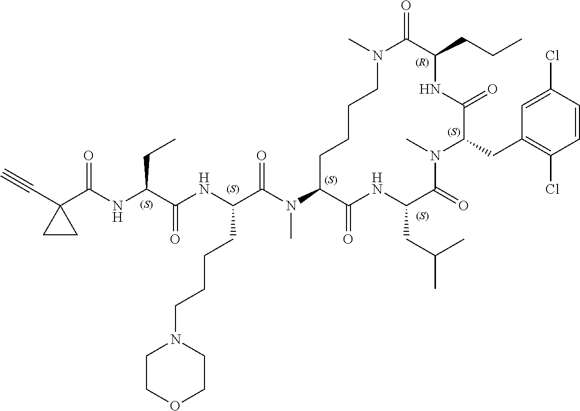 |
| 4 | 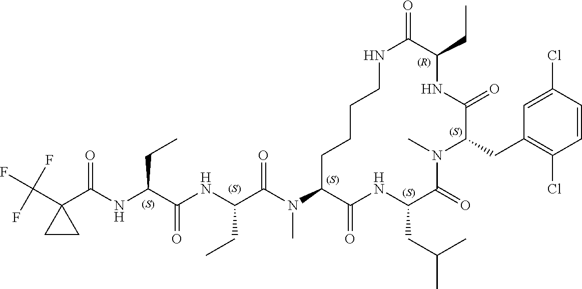 |
| 5 | 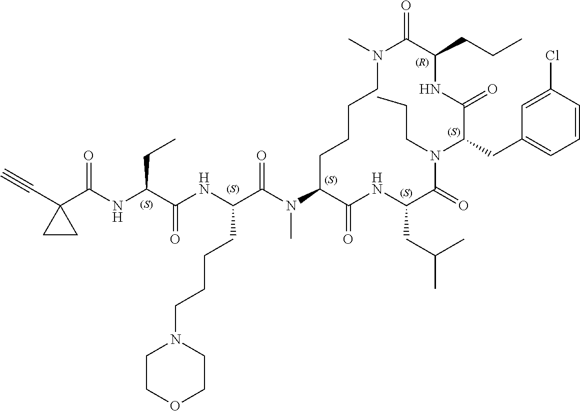 |
| 6 | 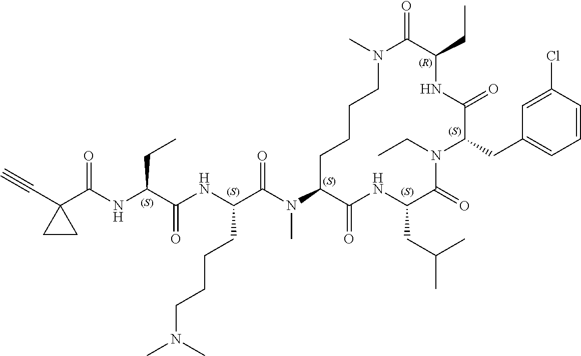 |
| 7 | 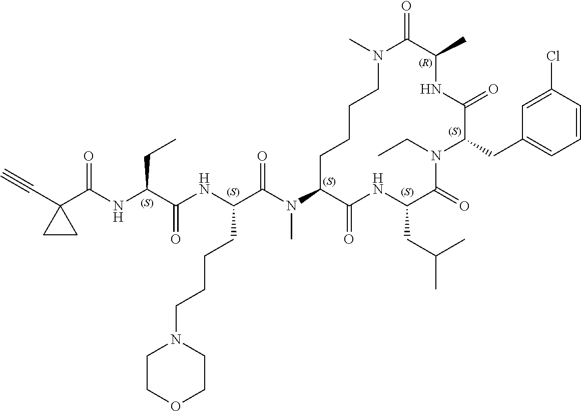 |
| 8 | 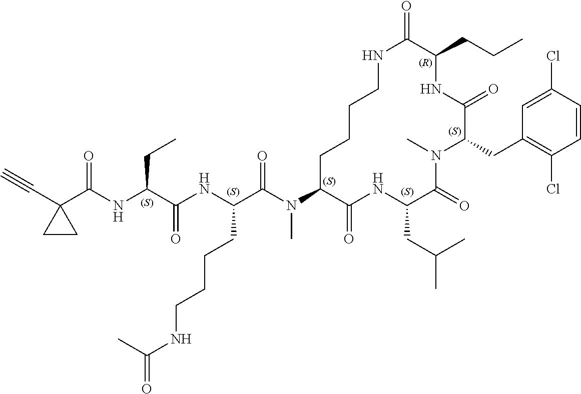 |
| 9 | 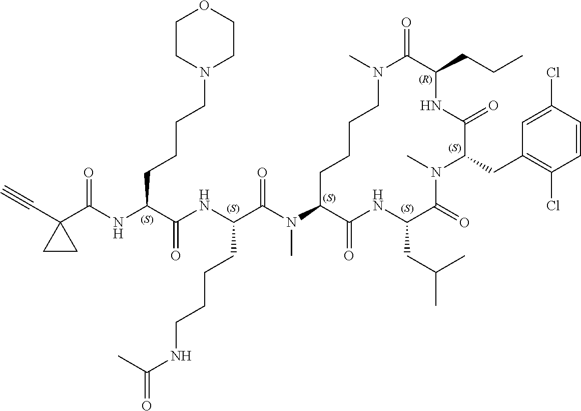 |
| 10 | 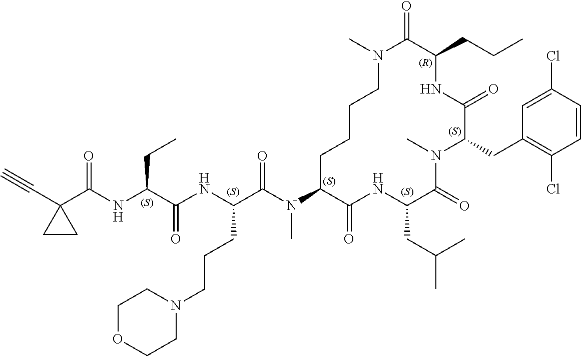 |
| 11 | 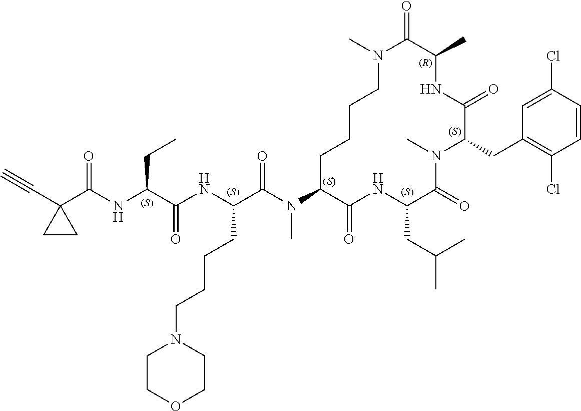 |
| 12 | 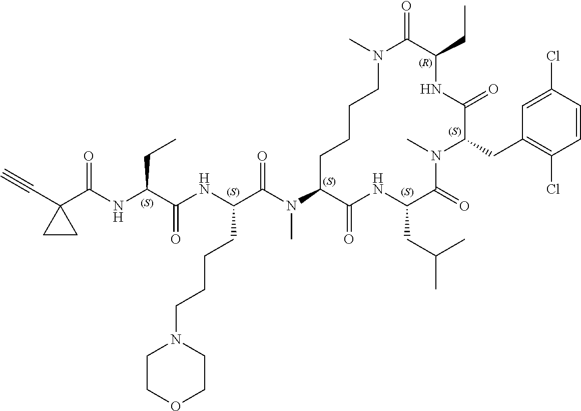 |
| 13 | 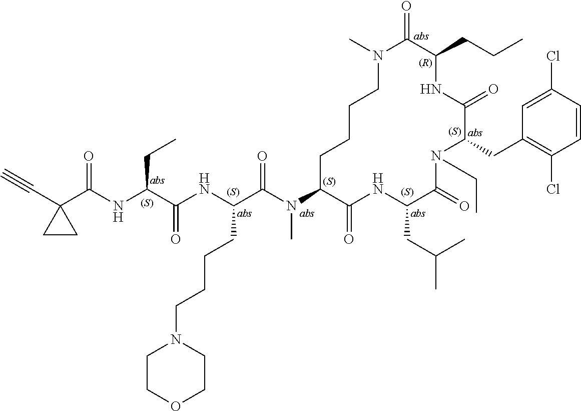 |
| 14 | 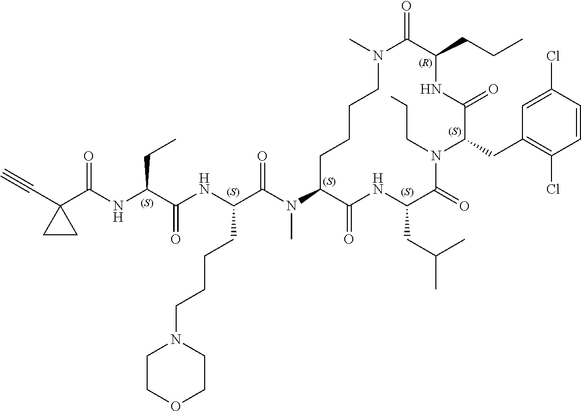 |
| 15 | 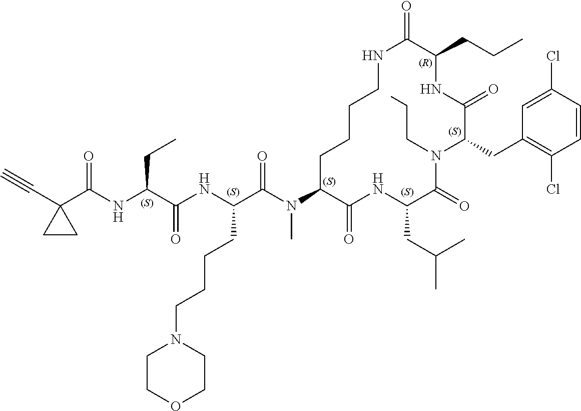 |
| 16 | 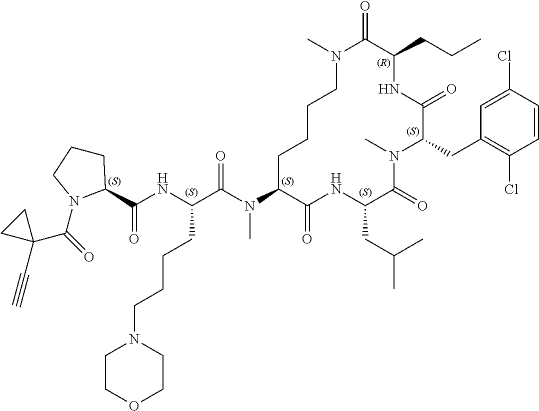 |
| 17 | 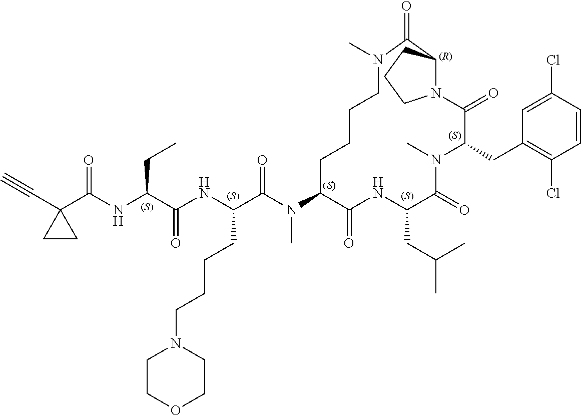 |
| 18 | 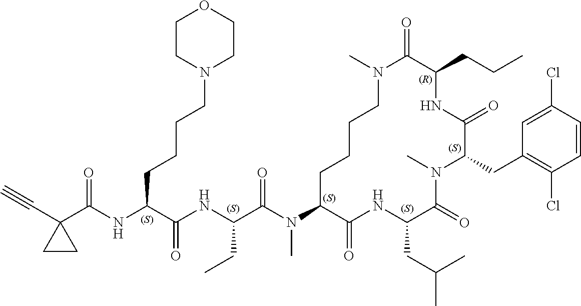 |
| 19 | 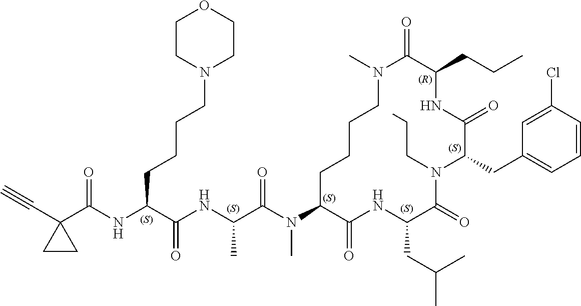 |
| 20 | 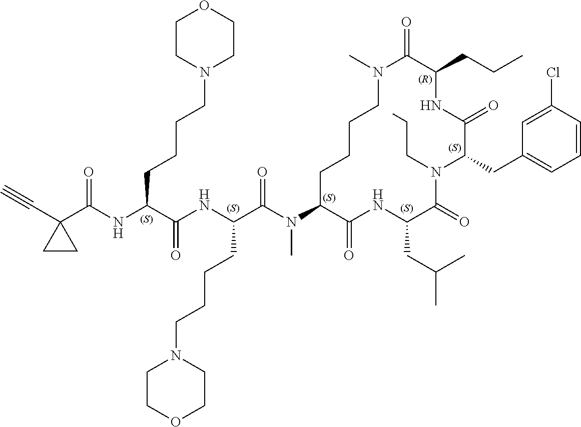 |
| 21 | 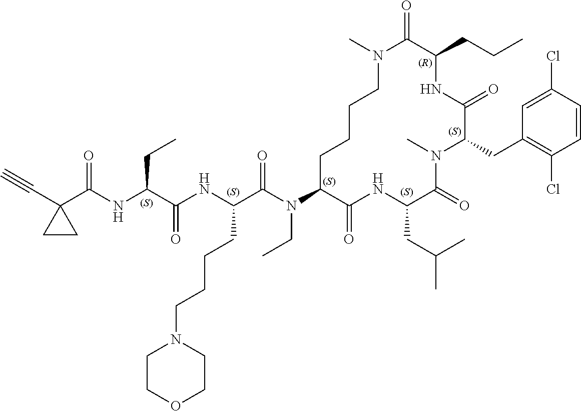 |
| 22 | 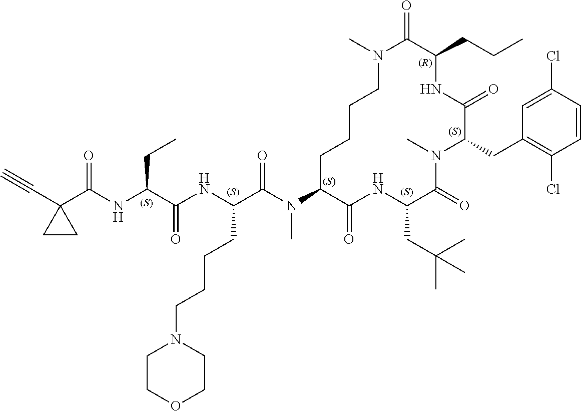 |
| 23 | 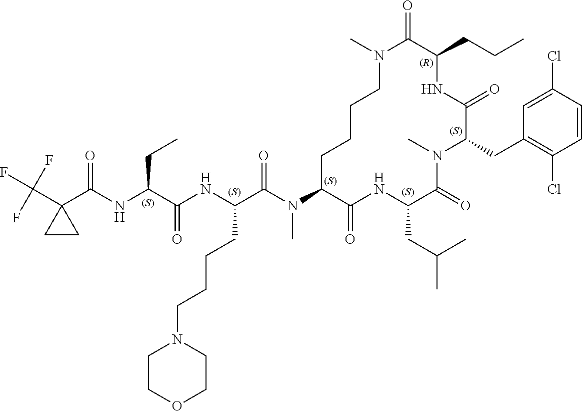 |
| 24 | 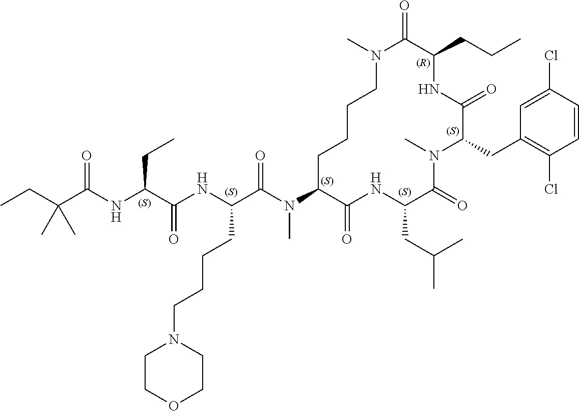 |
| 25 | 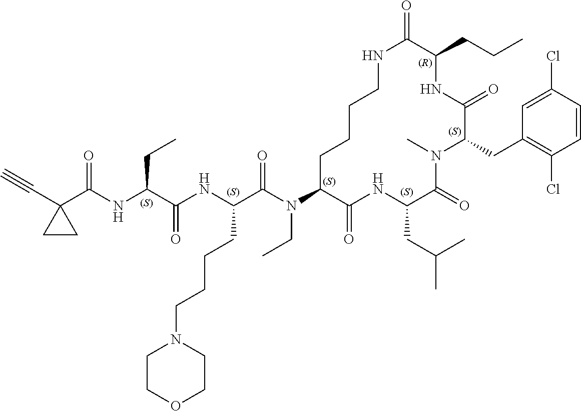 |
| 26 | 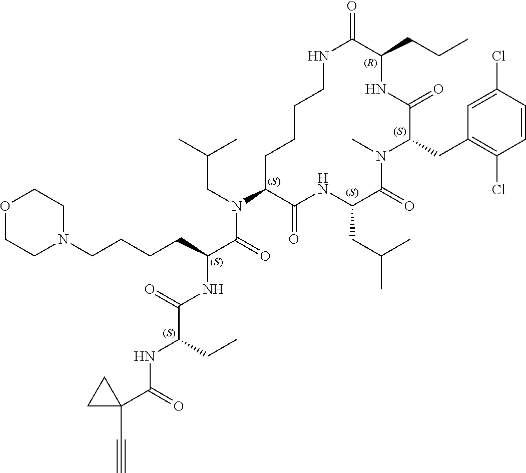 |
| 27 | 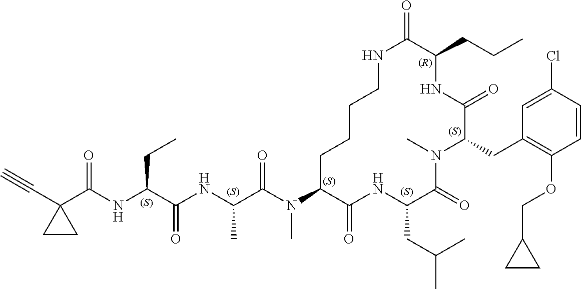 |
| 28 | 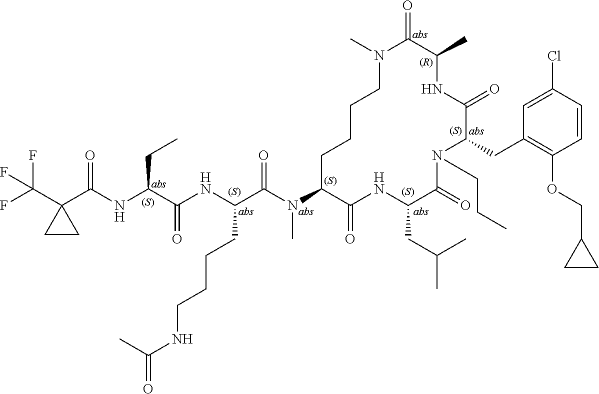 |
| 29 | 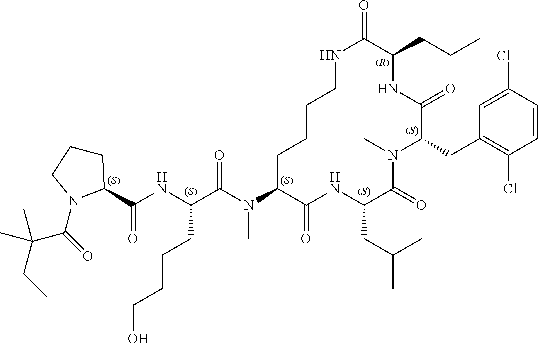 |
| 30 | 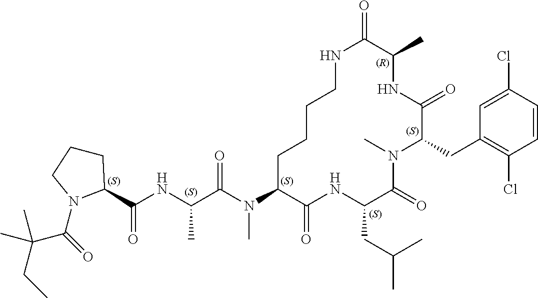 |
| 31 | 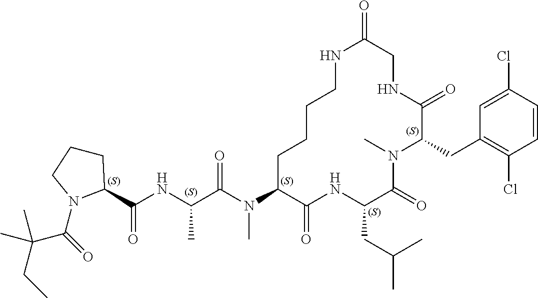 |
| 32 | 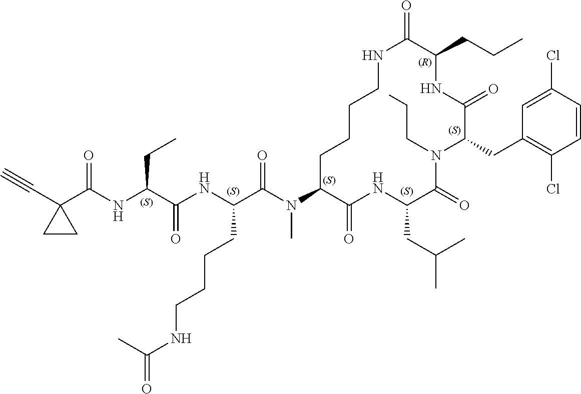 |
| 33 | 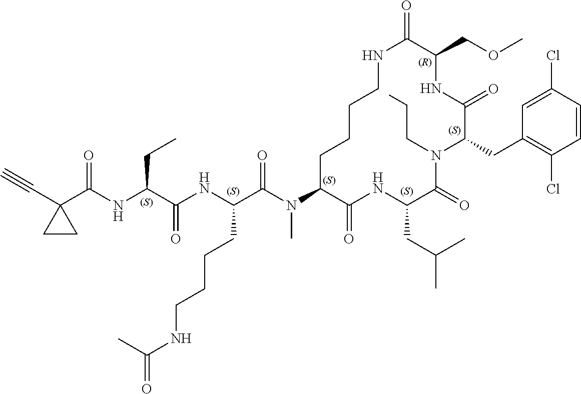 |
| 34 | 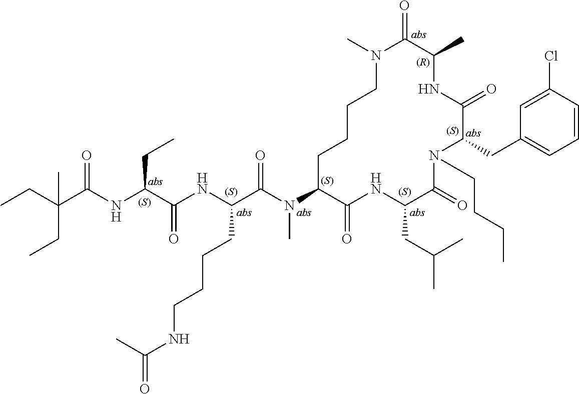 |
| 35 | 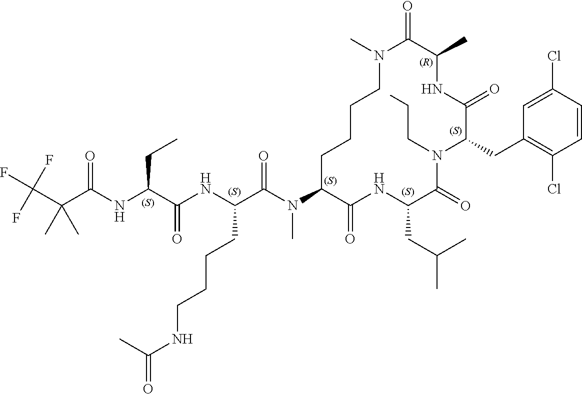 |
| 36 | 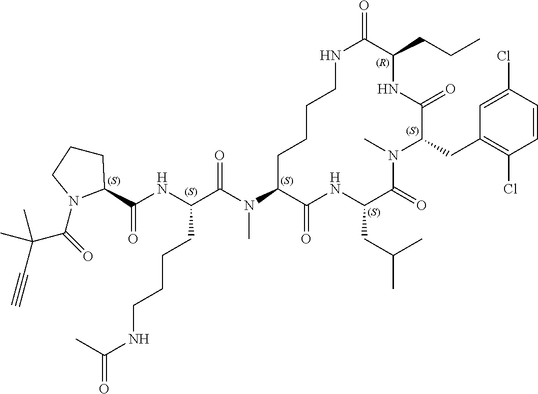 |
| 37 | 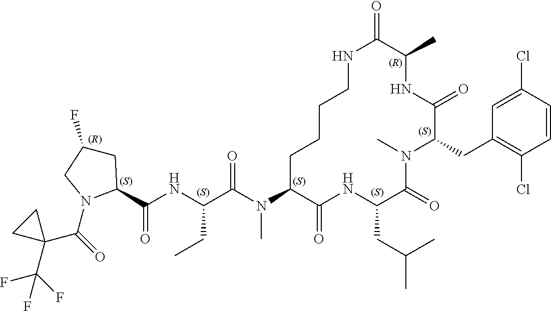 |
| 38 | 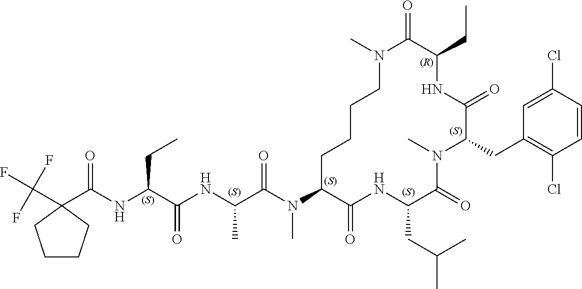 |
| 39 | 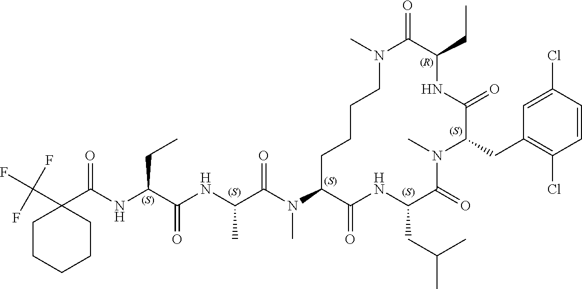 |
| 40 | 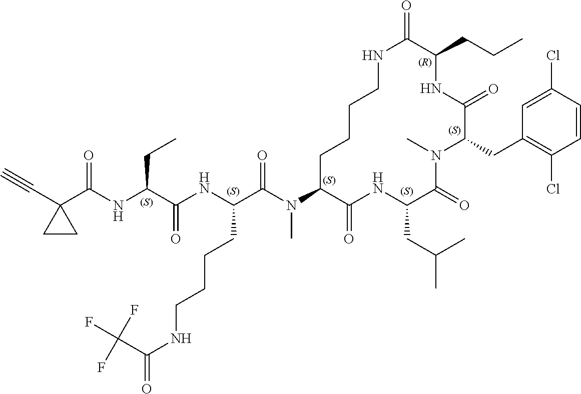 |
| 41 | 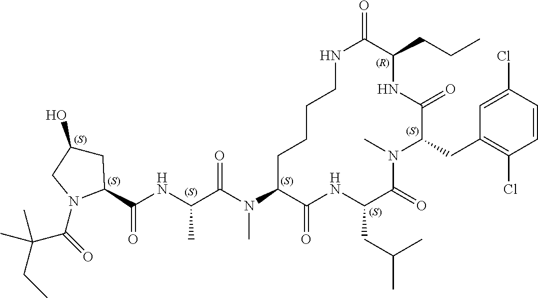 |
| 42 | 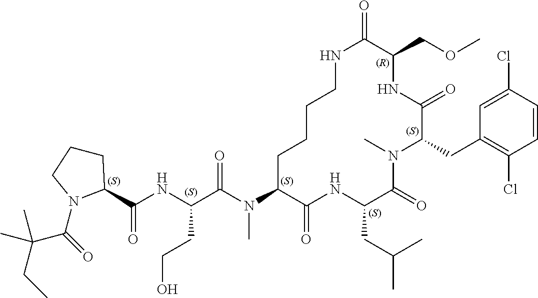 |
| 43 | 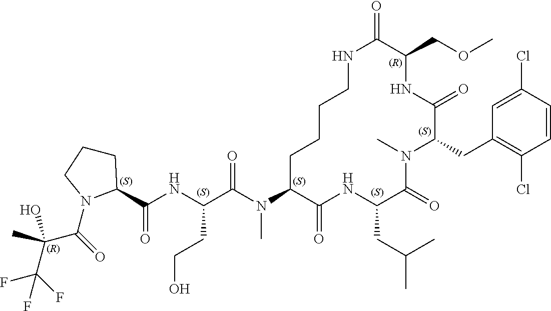 |
| 44 | 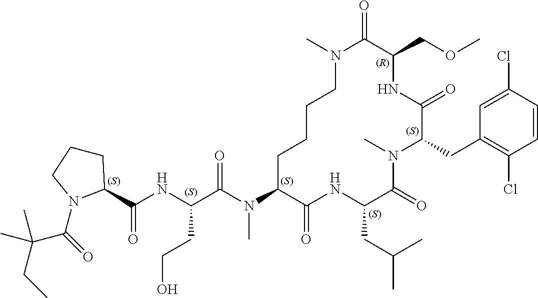 |
| 45 | 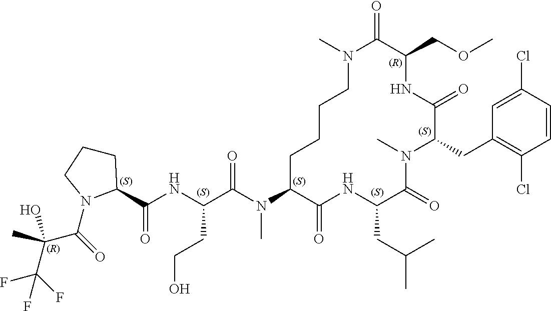 |
| 46 | 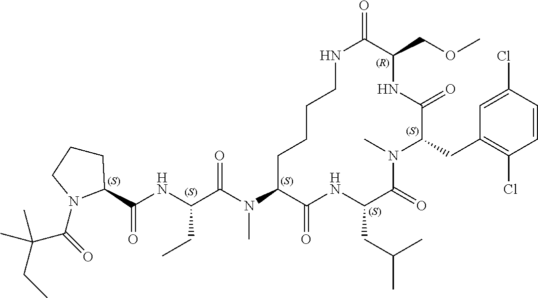 |
| 47 | 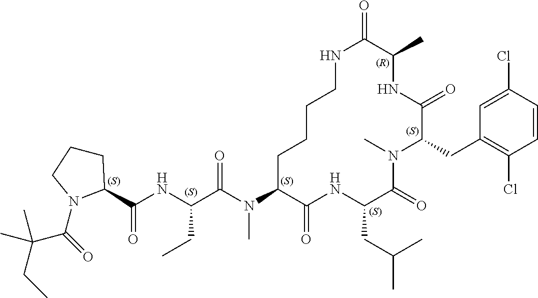 |
| 48 | 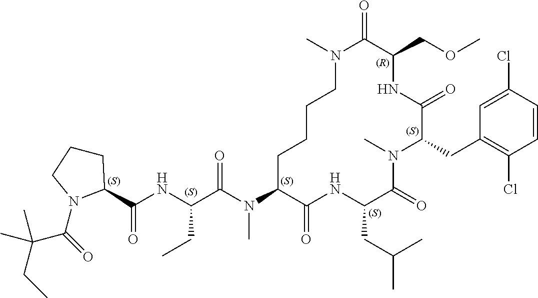 |
| 49 | 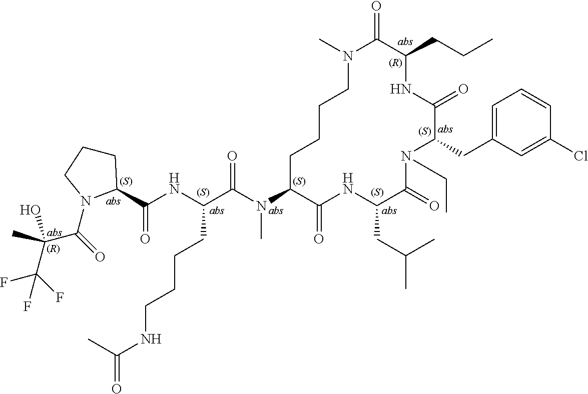 |
| 50 | 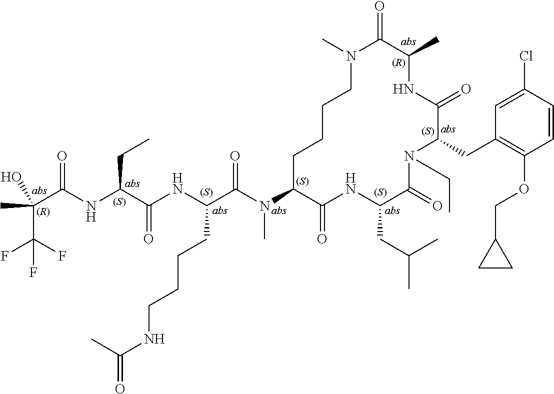 |
| 51 | 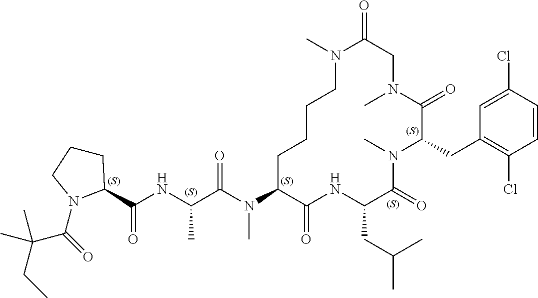 |
| 52 | 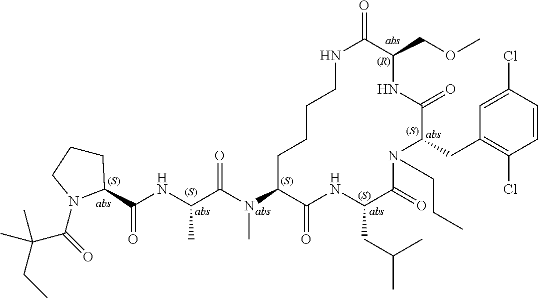 |
| 53 | 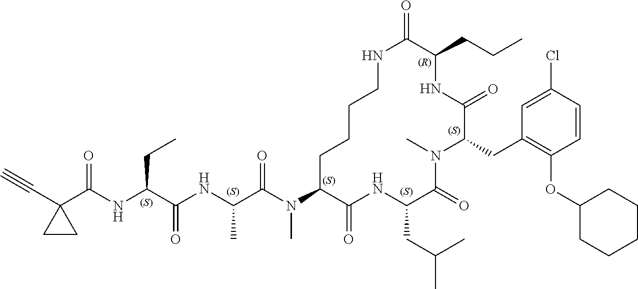 |
| 54 | 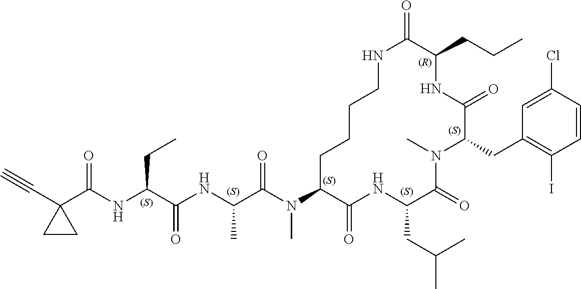 |
| 55 | 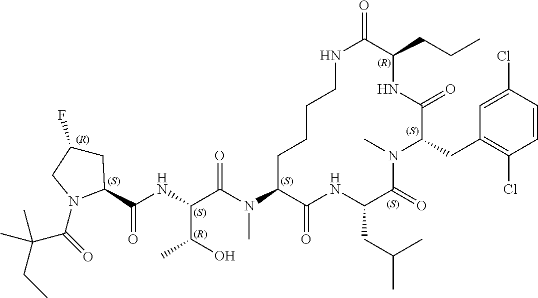 |
| 56 | 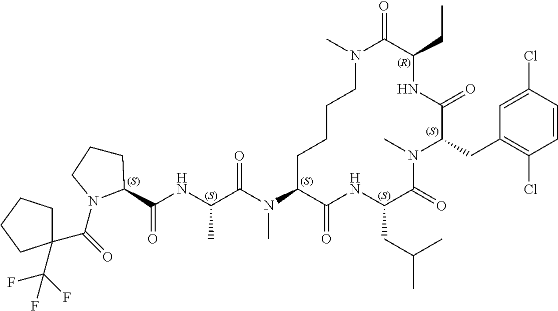 |
| 57 | 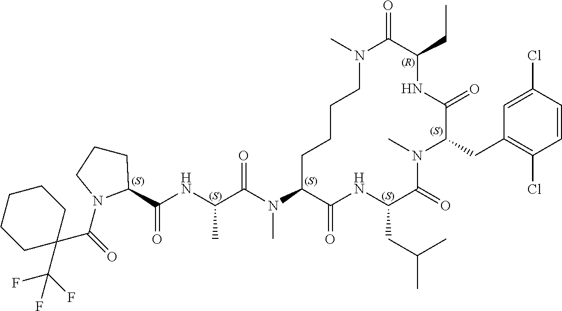 |
| 58 | 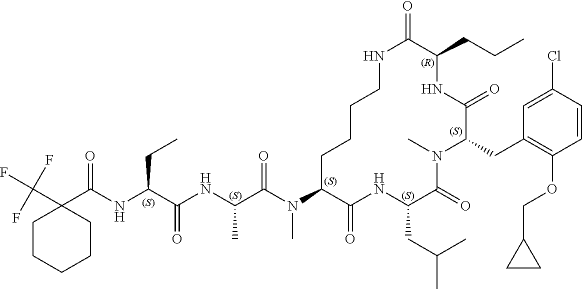 |
| 59 | 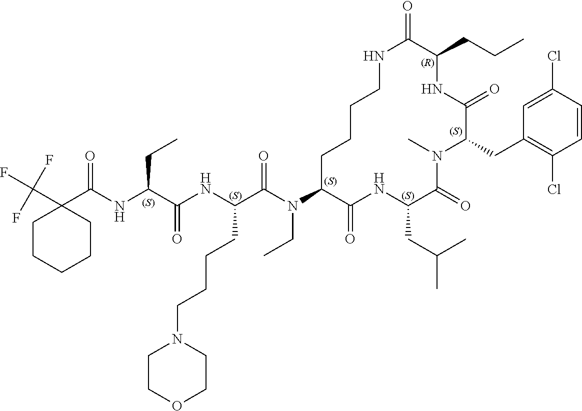 |
| 60 | 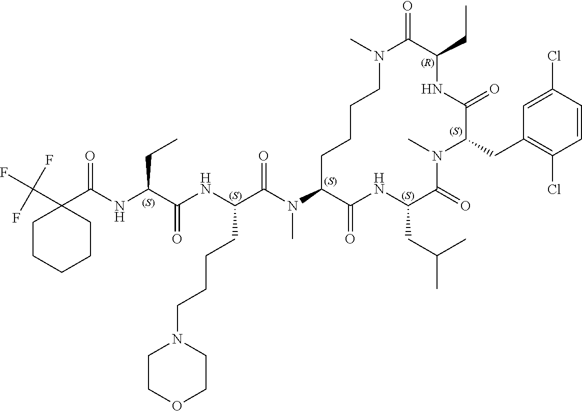 |
| 61 | 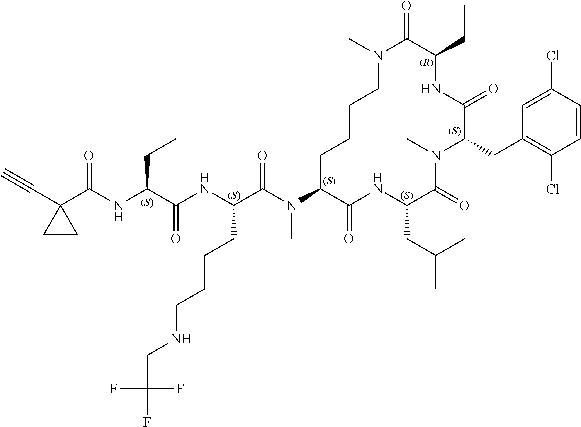 |
| 62 | 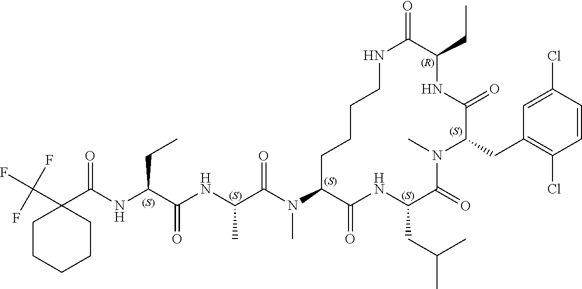 |
| 63 | 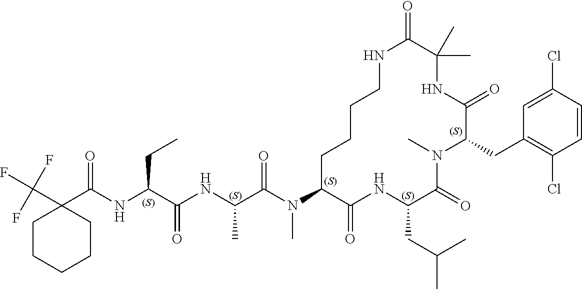 |
| 64 | 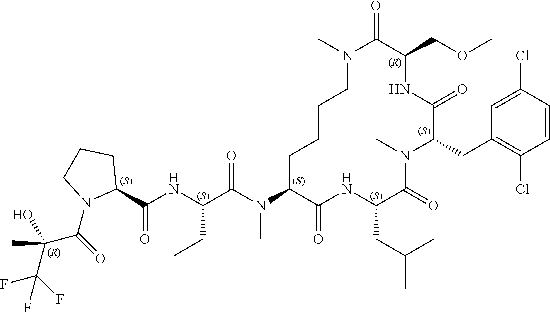 |
| 65 | 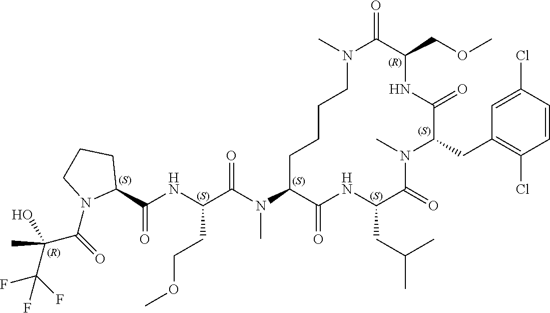 |
| 66 | 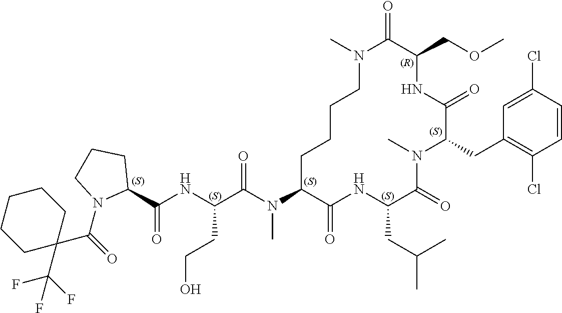 |
| 67 | 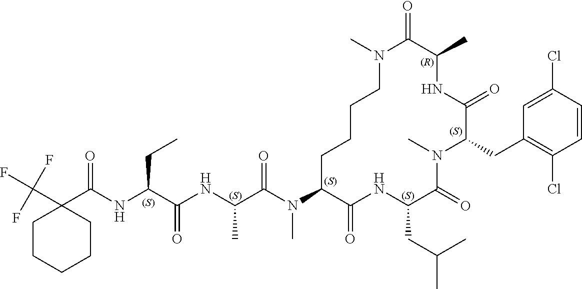 |
| 68 | 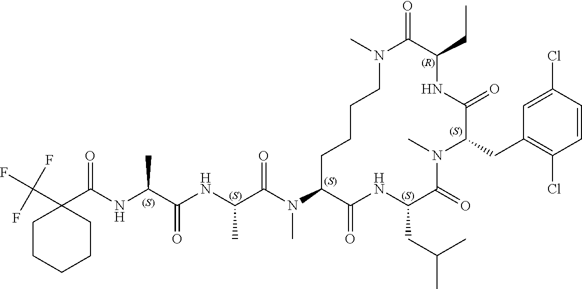 |
| 69 | 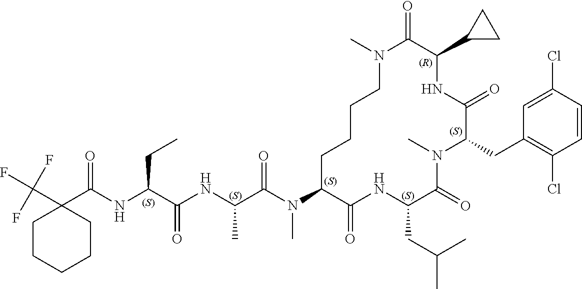 |
| 70 | 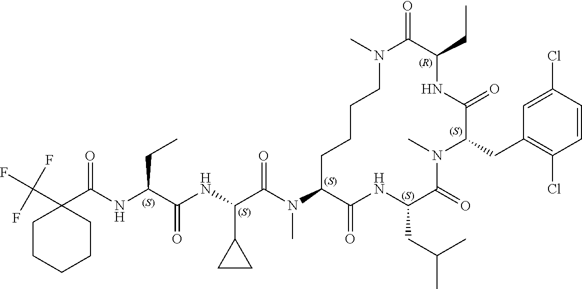 |
| 71 | 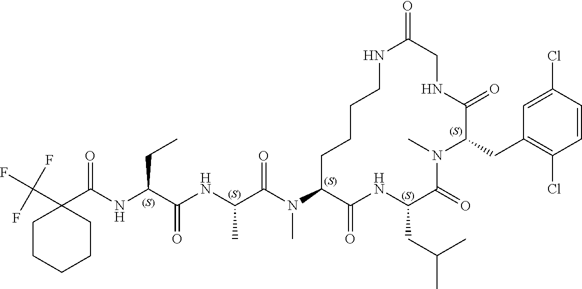 |
| 72 | 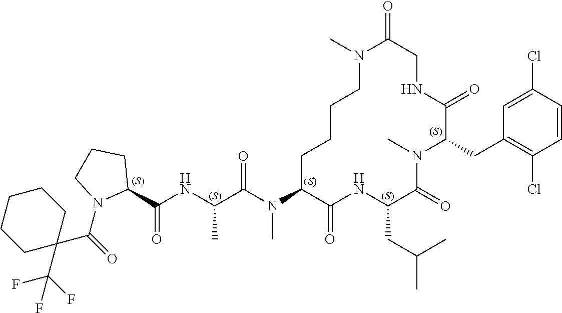 |
| 73 | 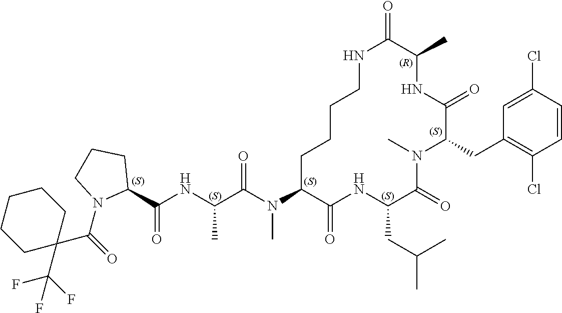 |
| 74 | 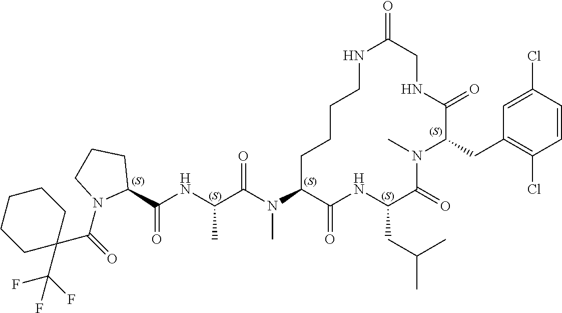 |
| 75 | 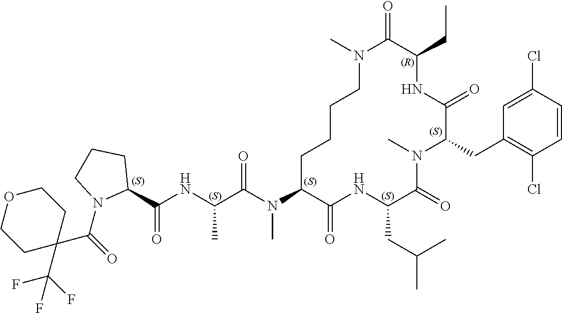 |
| 76 | 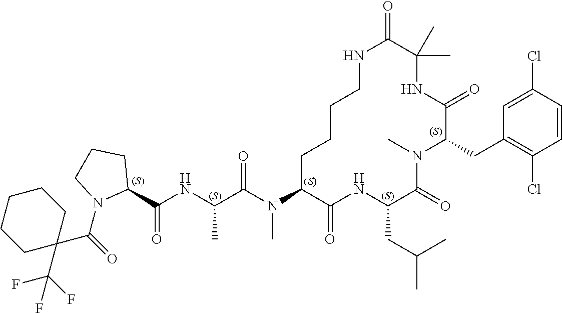 |
| 77 | 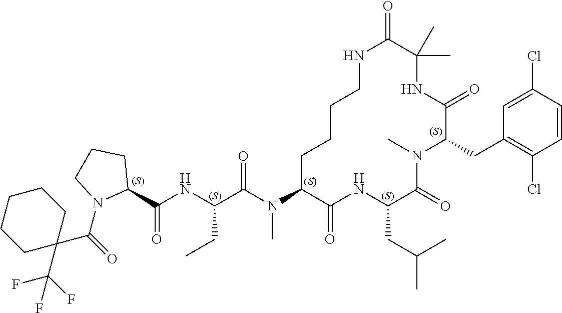 |
| 78 | 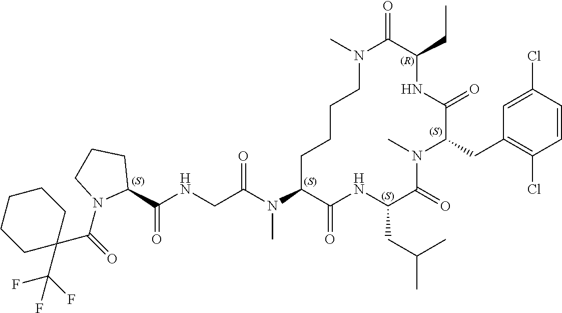 |
| 79 | 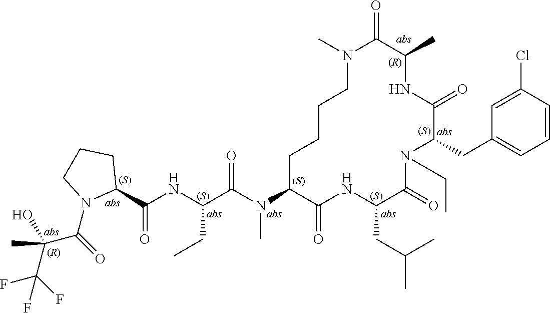 |
| 80 | 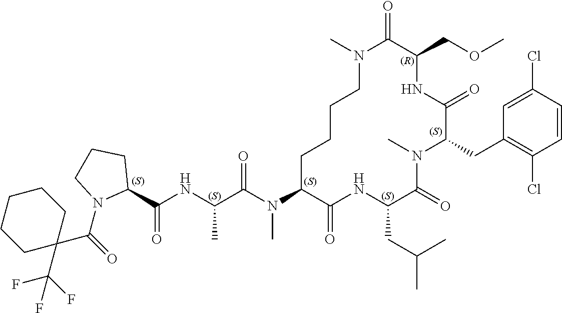 |
| 81 | 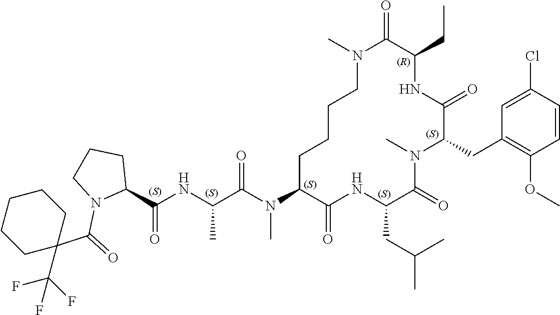 |
| 82 | 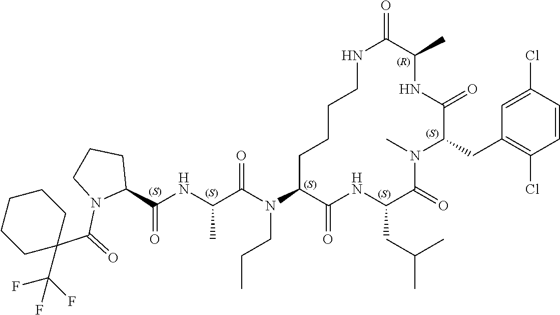 |
| 83 | 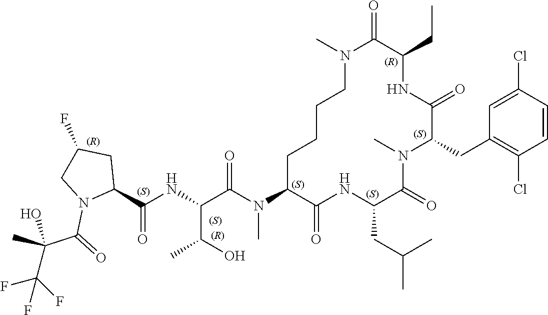 |
| 84 | 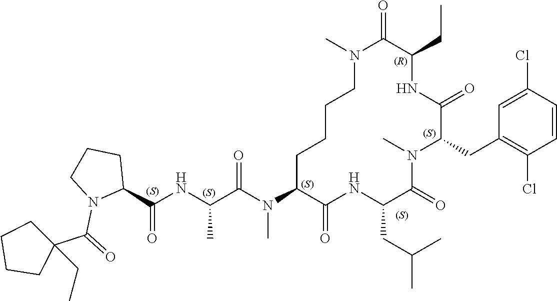 |
| 85 | 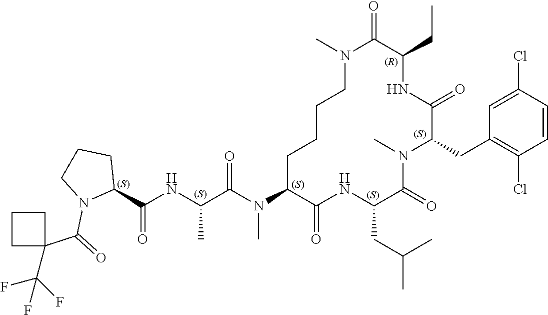 |
| 86 | 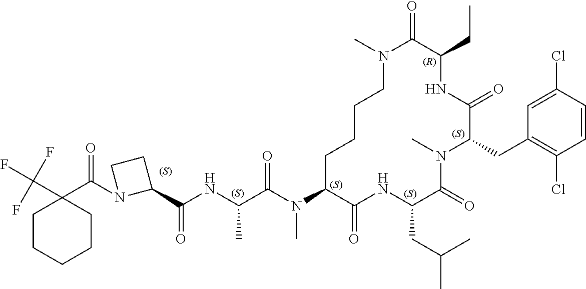 |
| 87 | 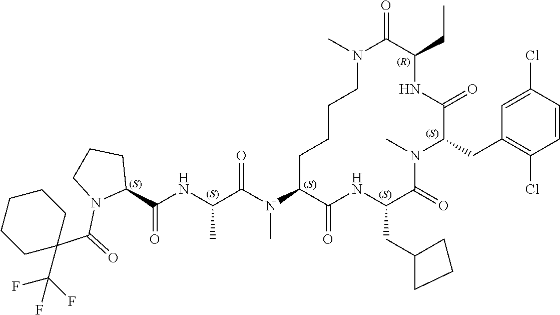 |
| 88 | 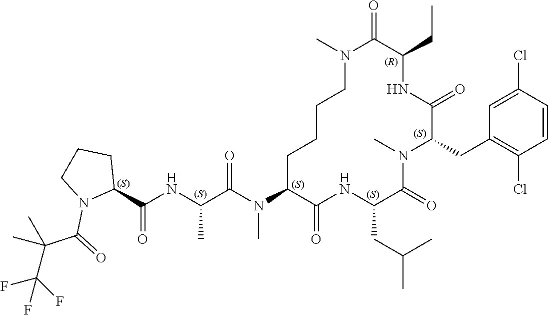 |
| 89 | 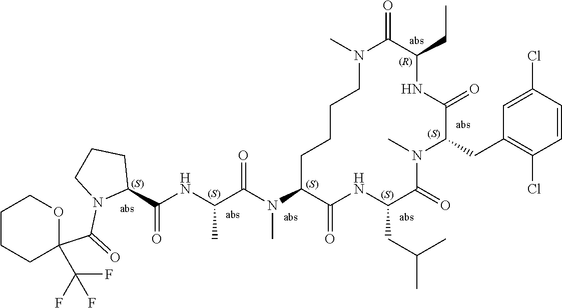 |
| 90 | 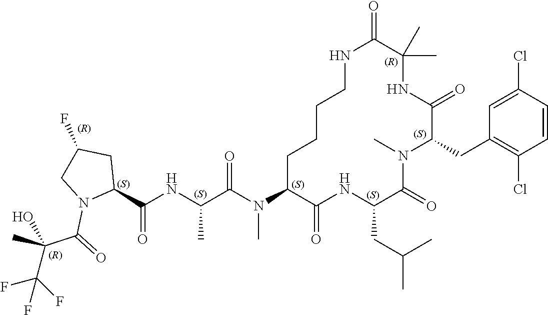 |
| 91 | 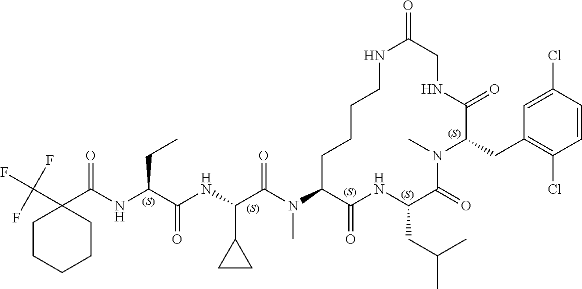 |
| 92 | 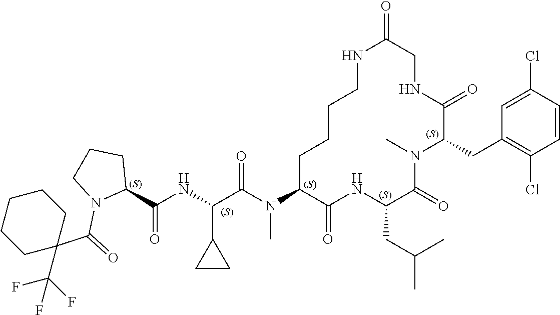 |
| 93 | 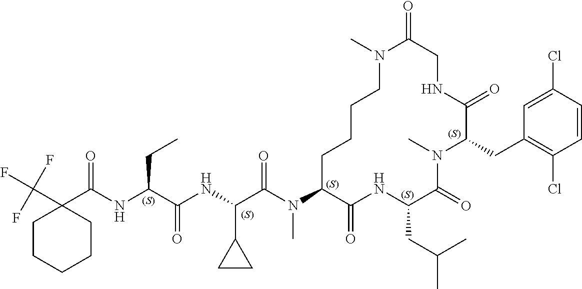 |
| 94 | 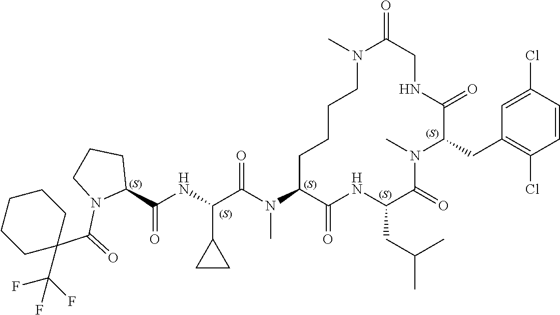 |
| 95 | 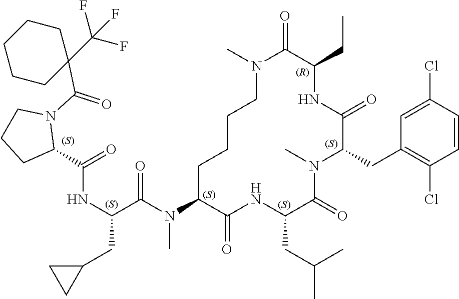 |
| 96 | 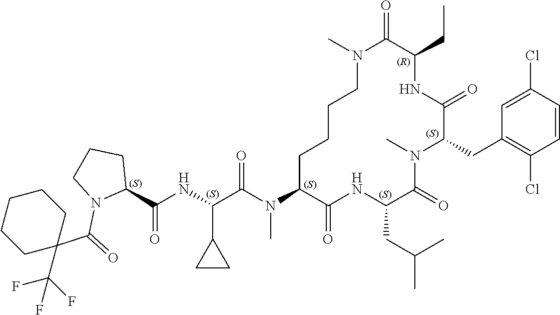 |
| 97 | 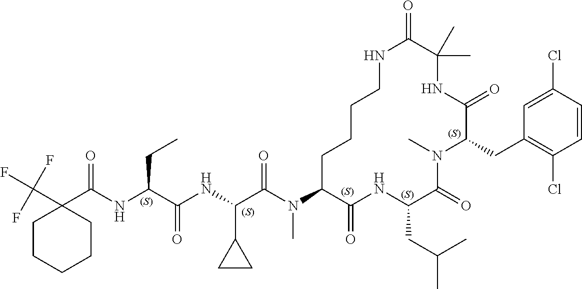 |
| 98 | 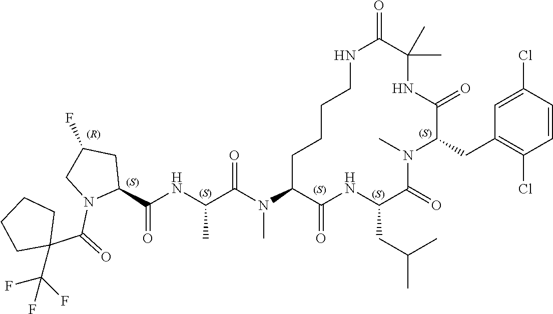 |
| 99 | 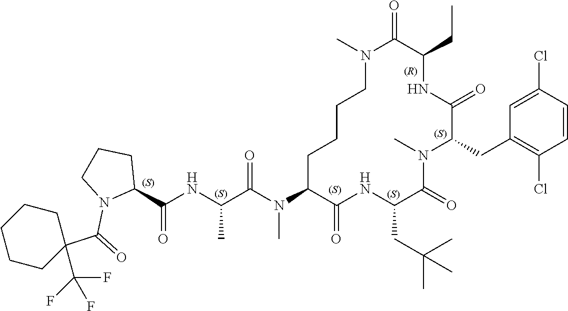 |
| 100 | 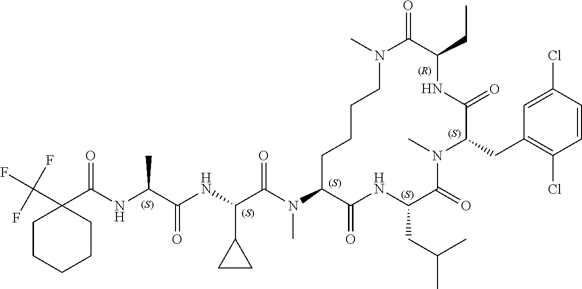 |
| 101 | 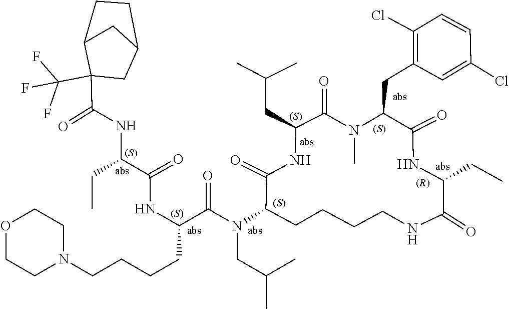 |
| 102 | 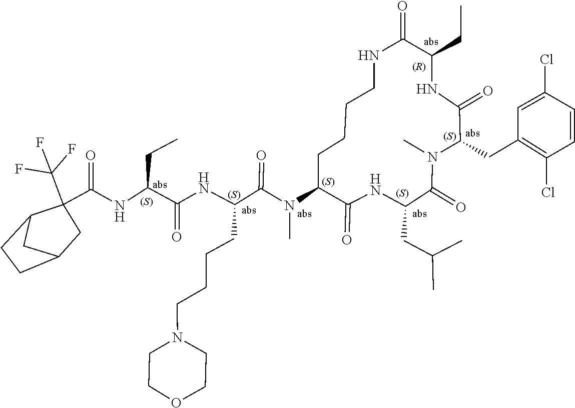 |
| 103 | 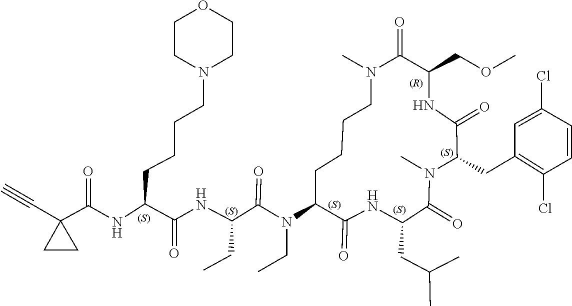 |
| 104 | 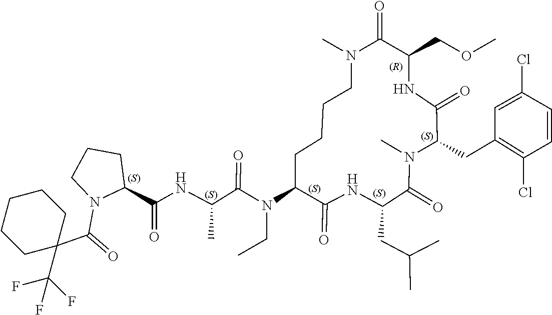 |
| 105 | 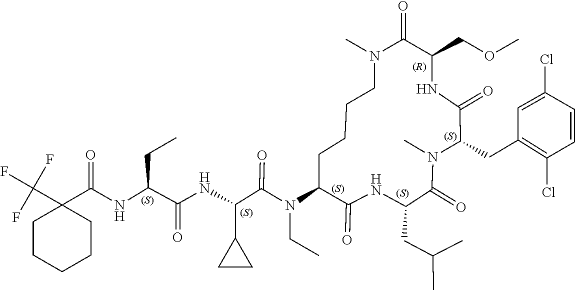 |
| 106 | 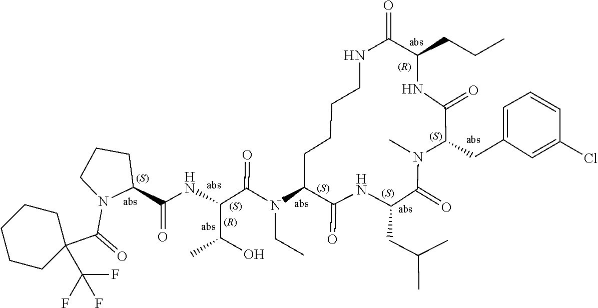 |
| 107 | 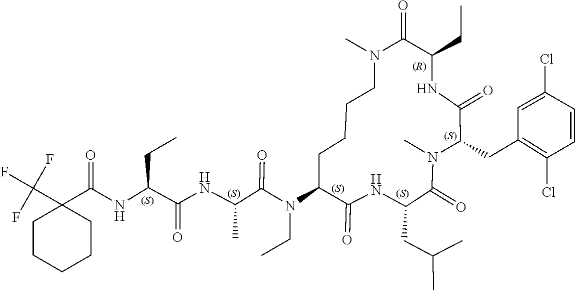 |
| 108 | 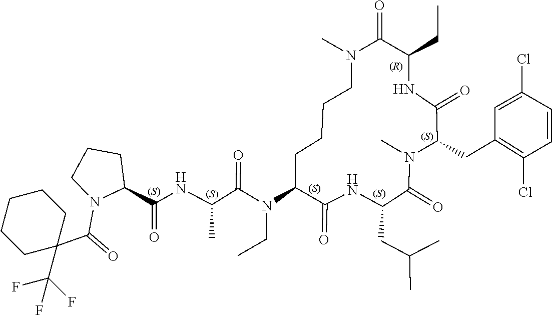 |
| 109 | 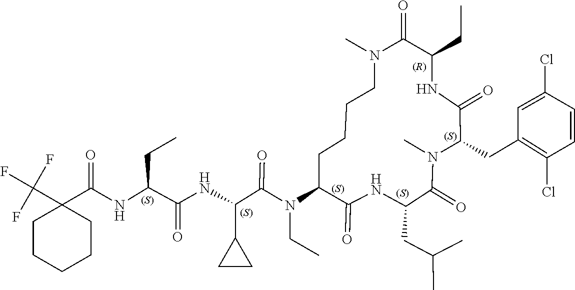 |
| 110 | 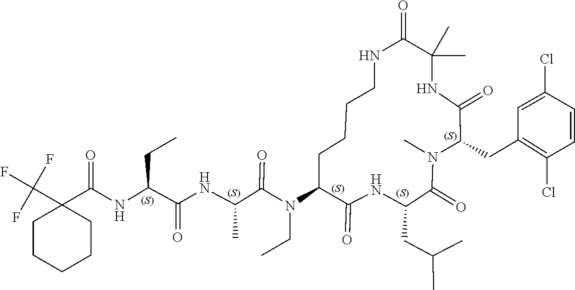 |
| 111 | 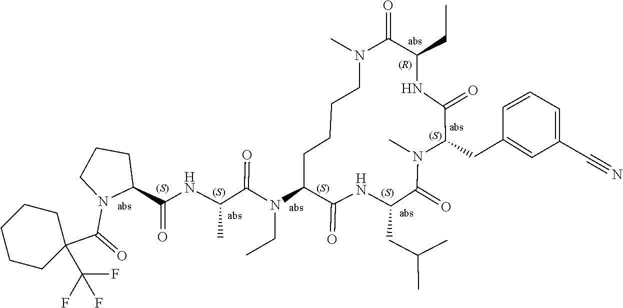 |
| 112 | 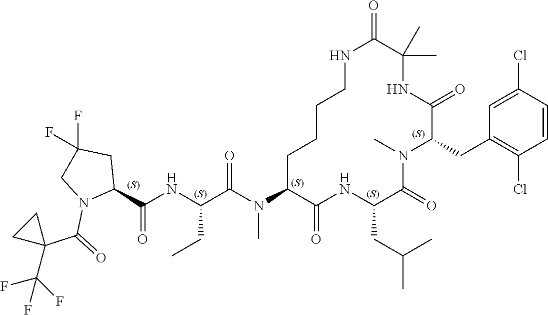 |
| 113 | 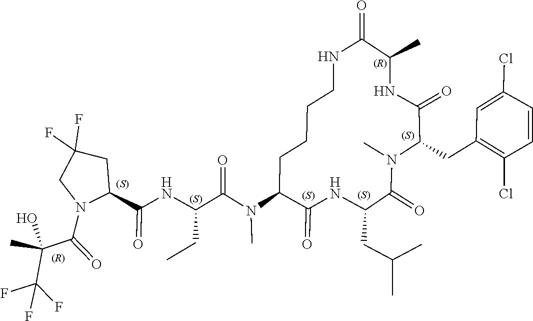 |
| 114 | 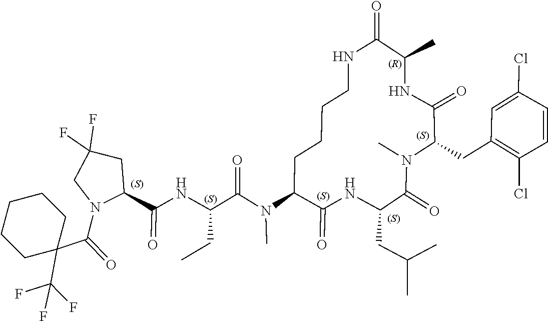 |
| 115 | 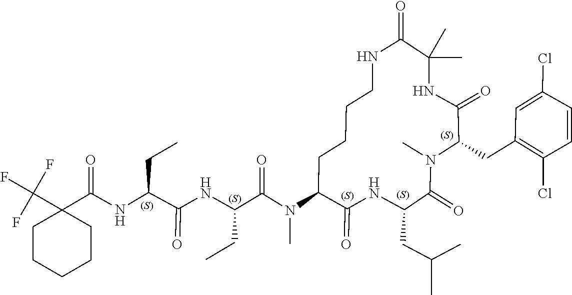 |
| 116 | 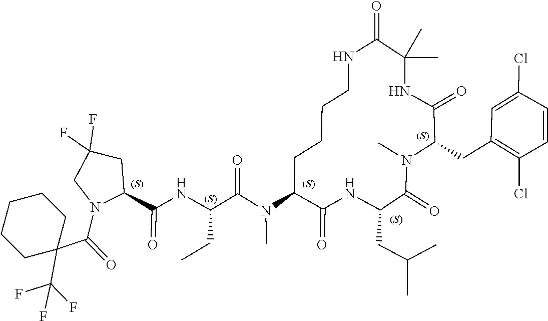 |
| 117 | 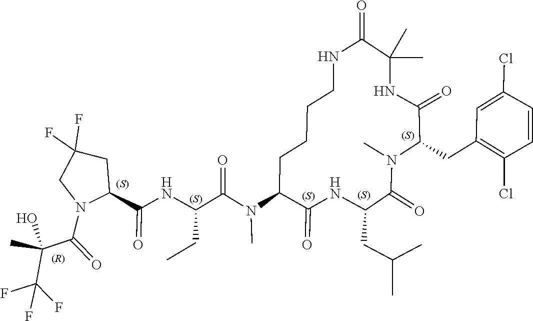 |
| 118 | 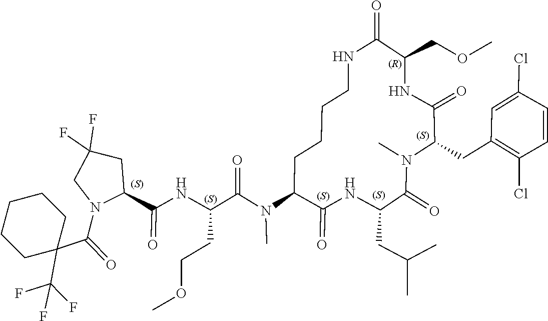 |
| 119 | 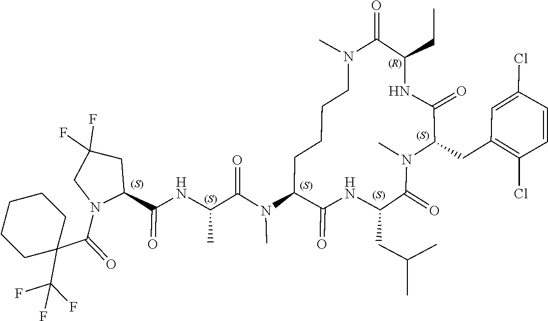 |
| 120 | 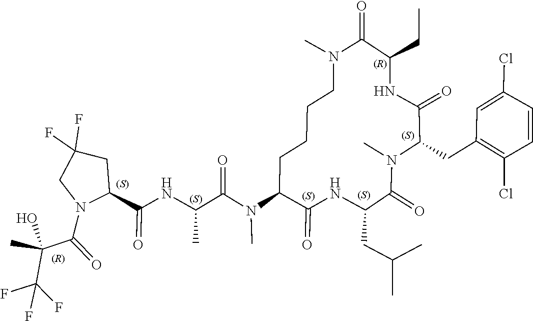 |
| 121 | 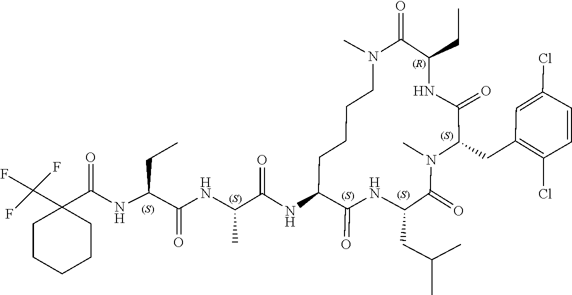 |
| 122 | 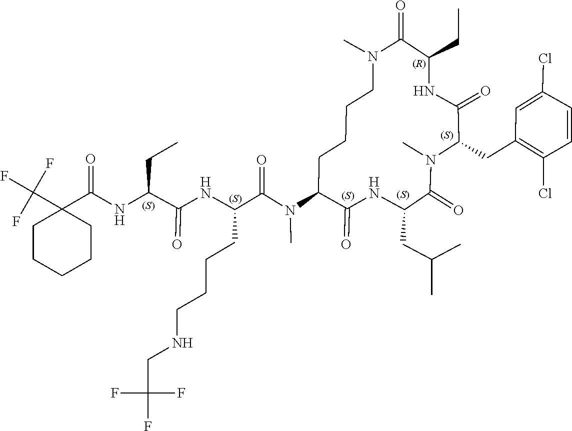 |
| 123 | 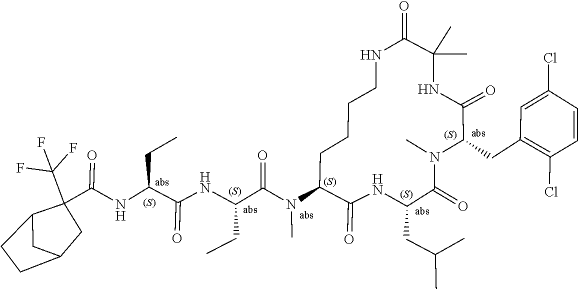 |
| 124 | 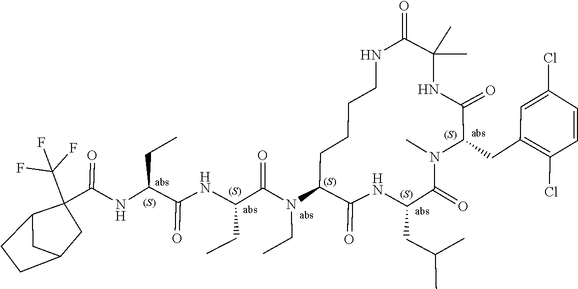 |
| 125 | 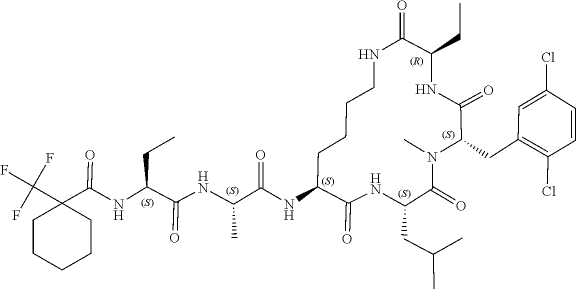 |
| 126 | 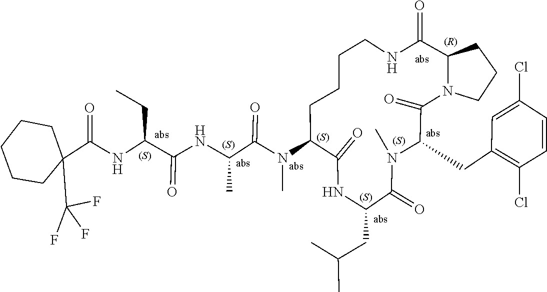 |
| 127 | 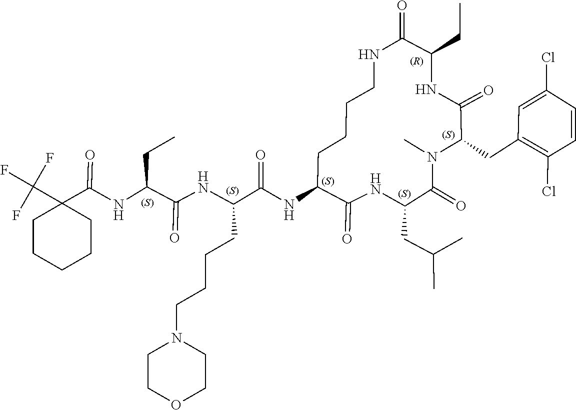 |
| 128 | 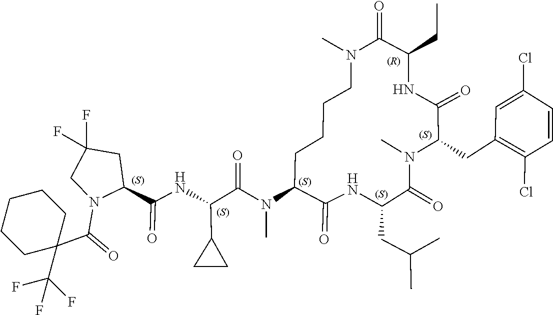 |
| 129 | 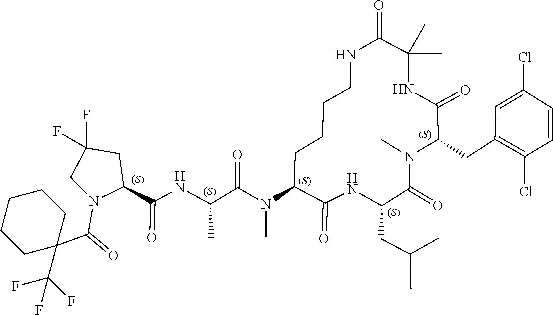 |
| 130 | 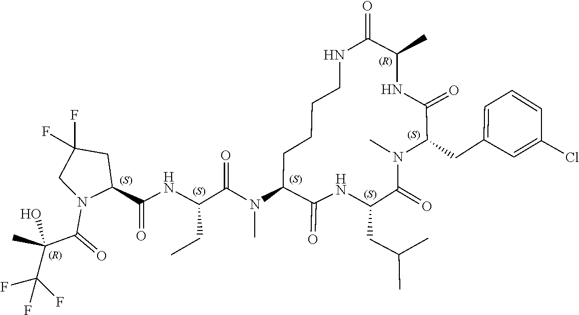 |
| 131 | 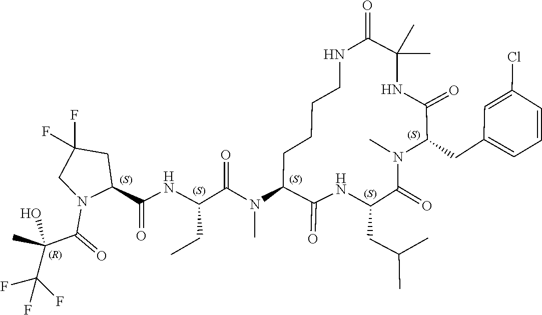 |
| 132 | 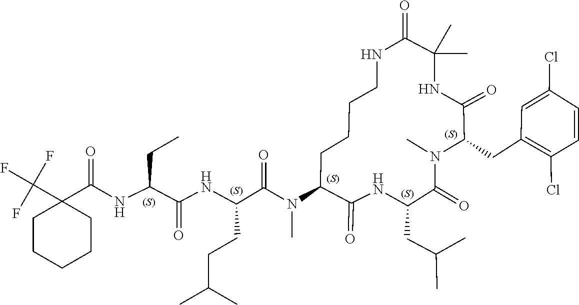 |
| 133 | 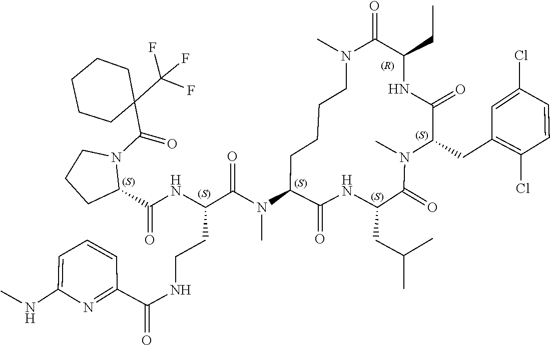 |
| 134 | 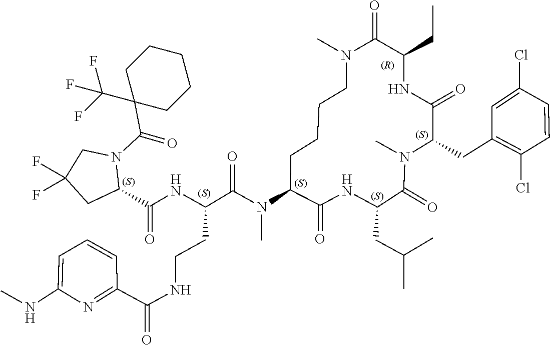 |
| 135 | 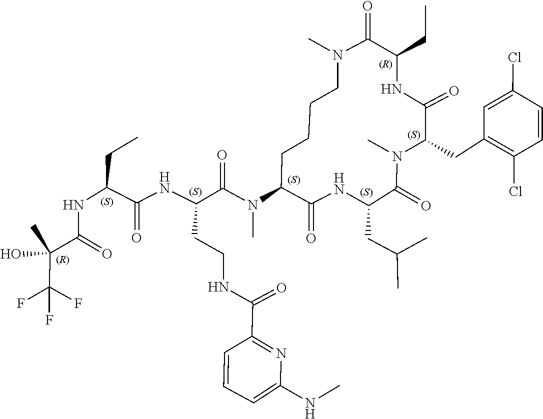 |
| 136 | 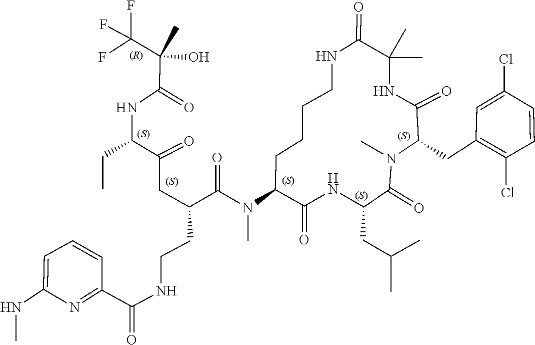 |
| 137 | 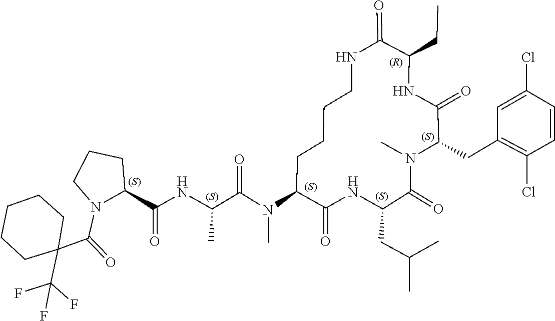 |
| 138 | 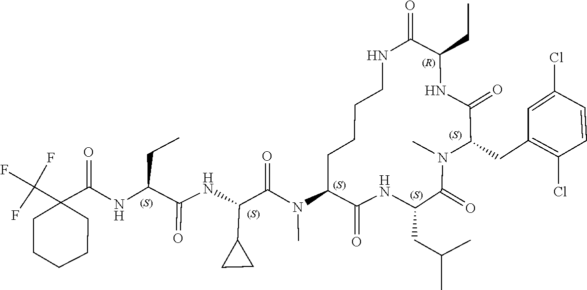 |
| 139 | 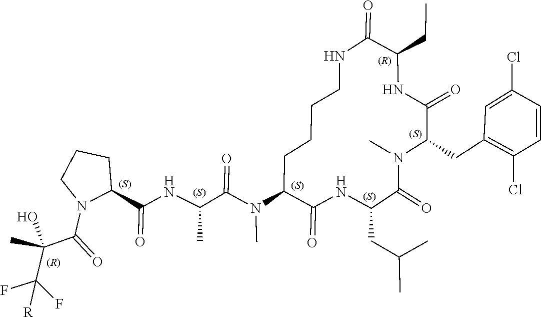 |
| 140 | 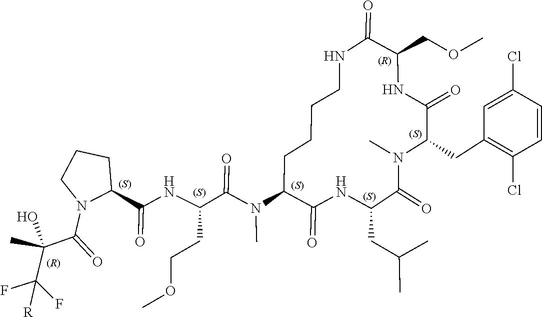 |
| 141 | 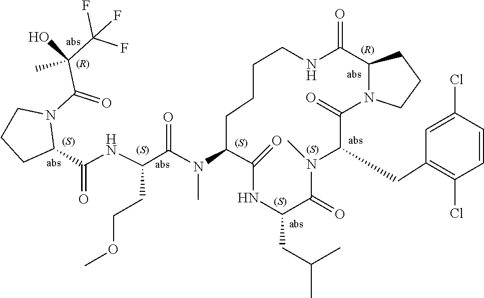 |
| 142 | 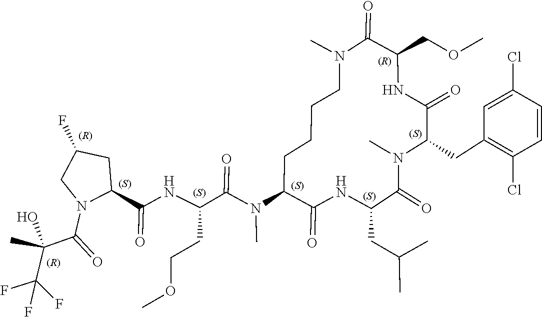 |
| 143 | 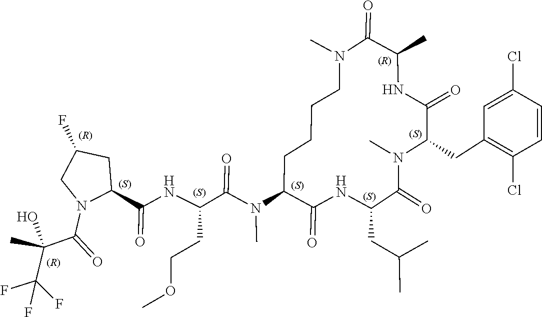 |
| 144 | 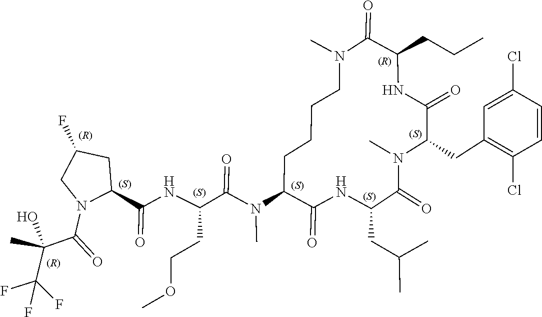 |
| 145 | 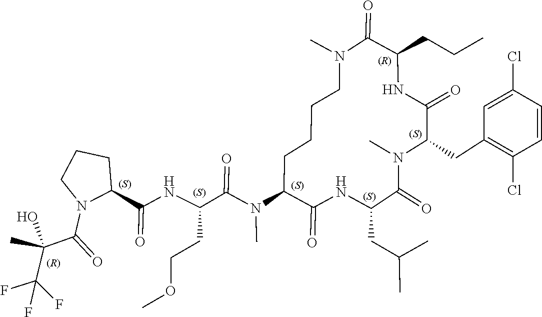 |
| 146 | 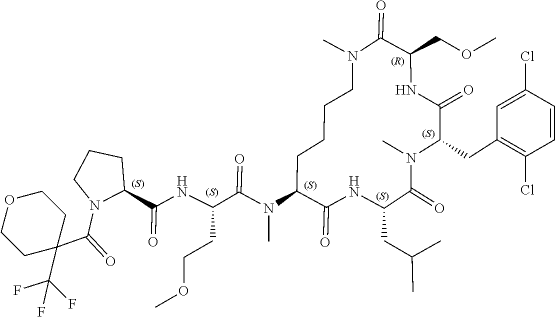 |
| 147 | 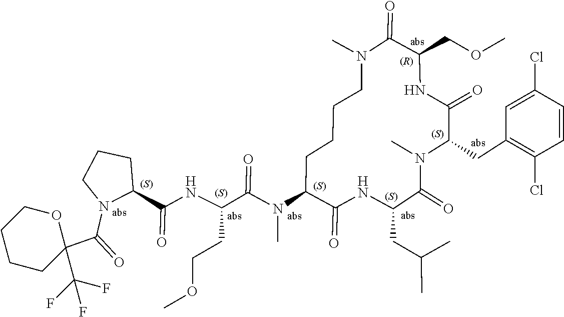 |
| 148 | 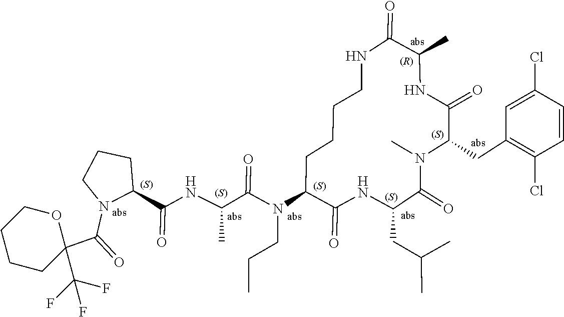 |
| 149 | 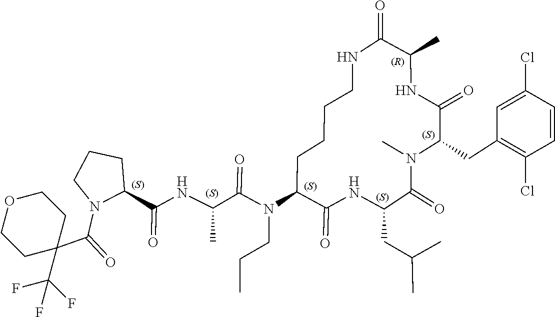 |
| 150 | 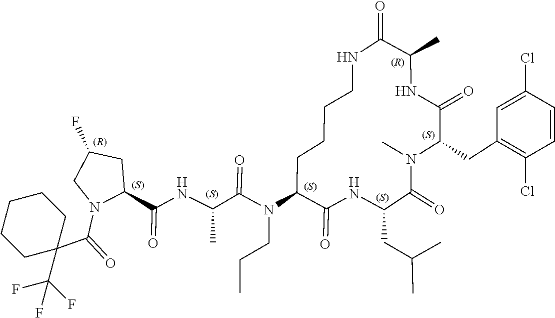 |
| 151 | 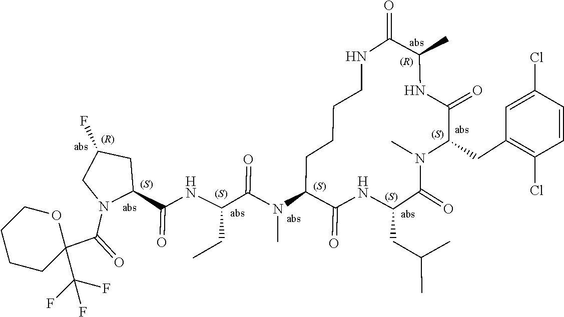 |
| 152 | 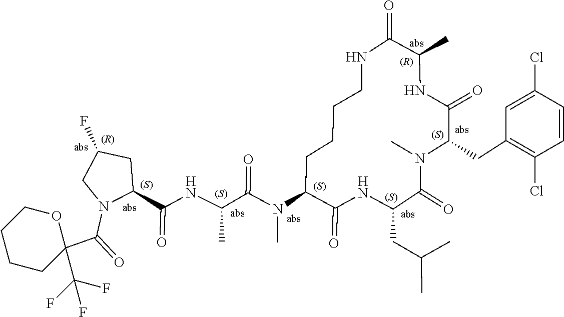 |
| 153 | 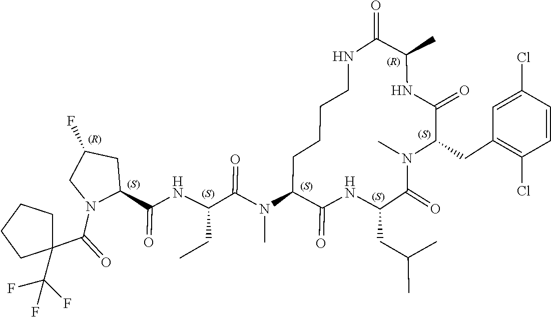 |
| 154 | 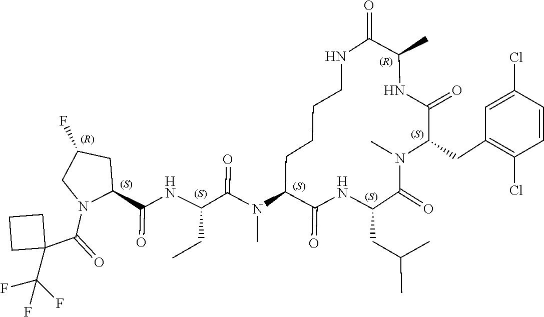 |
| 155 | 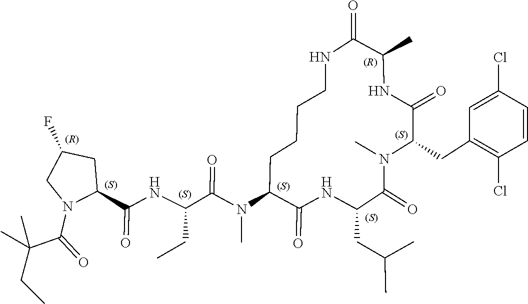 |
| 156 | 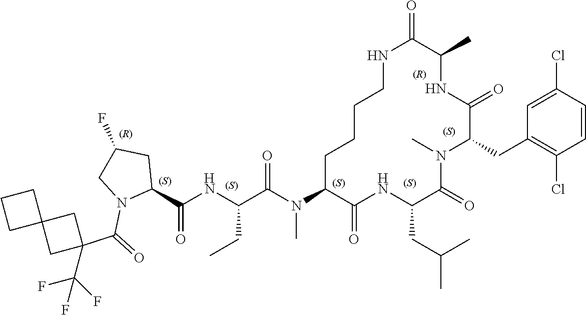 |
| 157 | 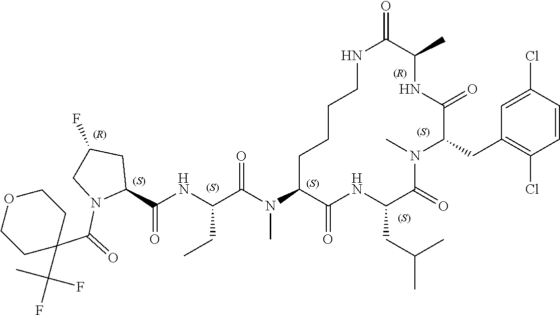 |
| 158 | 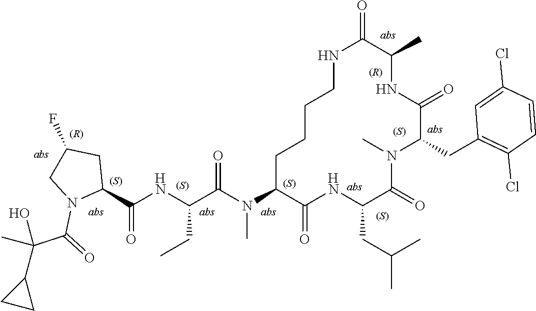 |
| 159 | 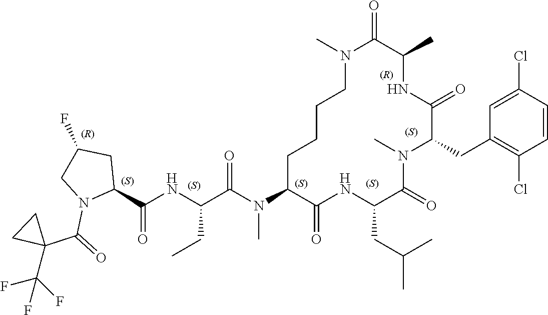 |
| 160 | 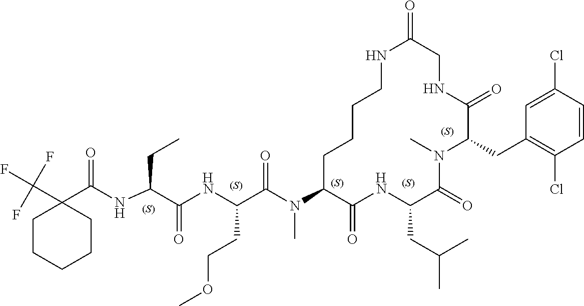 |
| 161 | 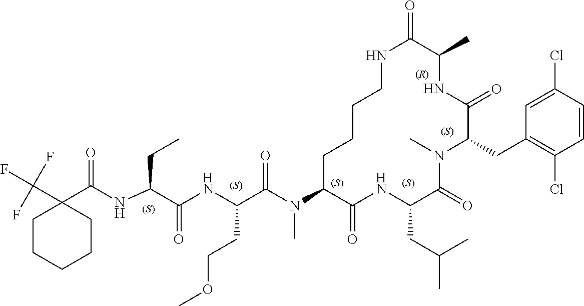 |
| 162 | 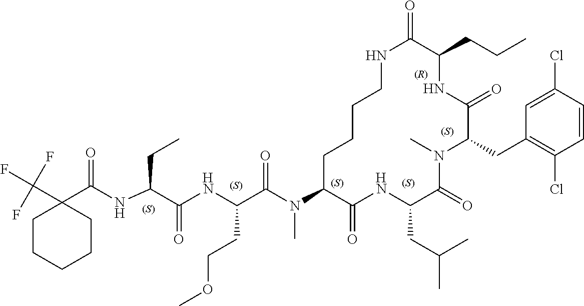 |
| 163 | 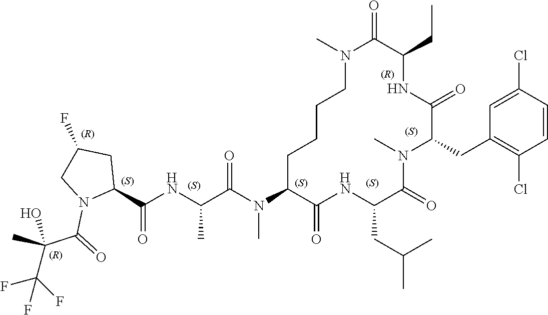 |
| 164 | 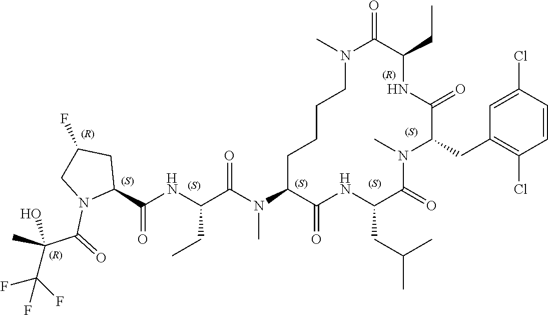 |
| 165 | 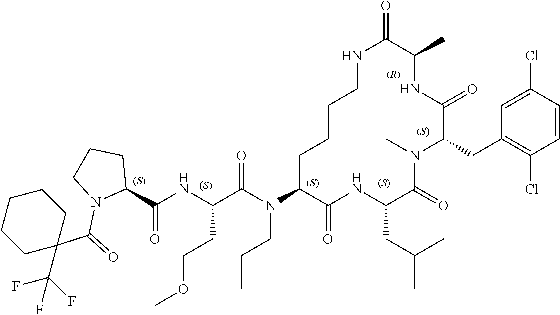 |
| 166 | 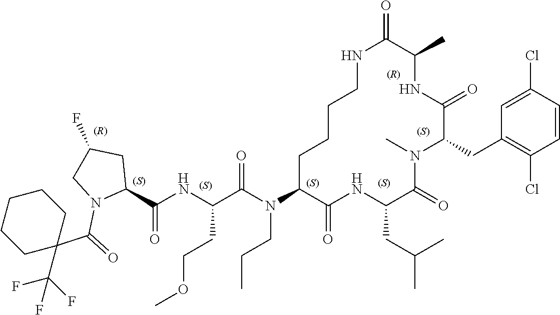 |
| 167 | 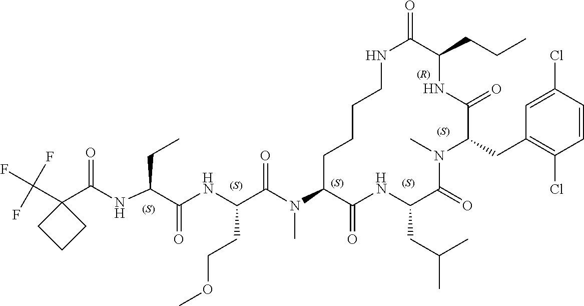 |
| 168 | 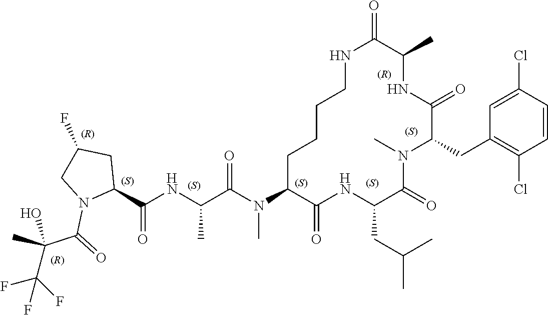 |
| 169 | 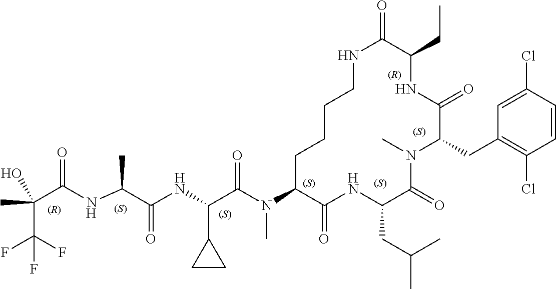 |
| 170 | 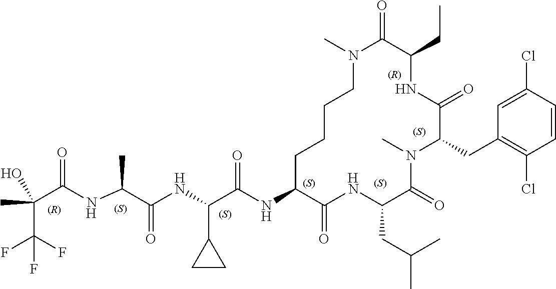 |
| 171 | 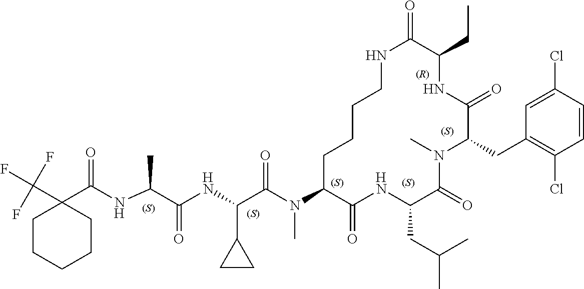 |
| 172 | 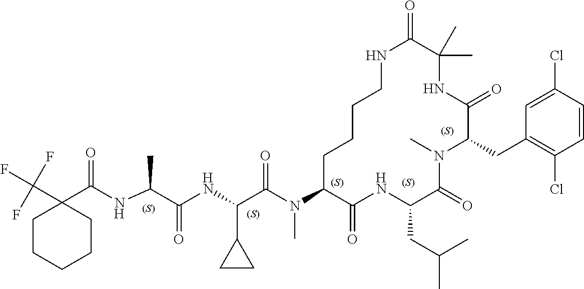 |
| 173 | 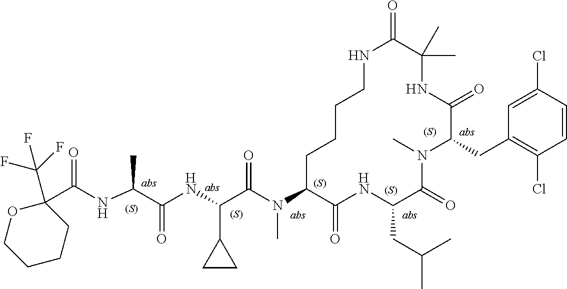 |
| 174 | 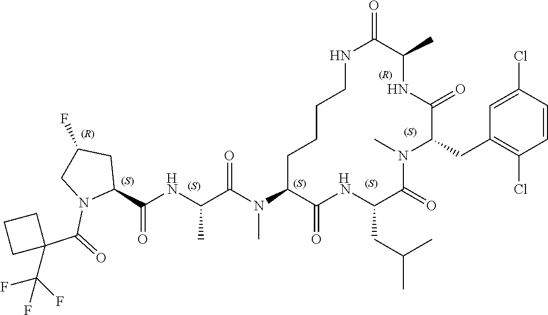 |
| 175 | 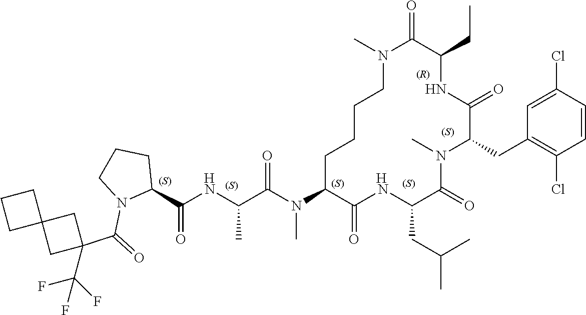 |
| 176 | 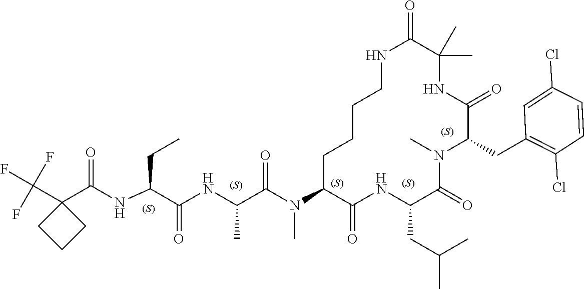 |
| 177 | 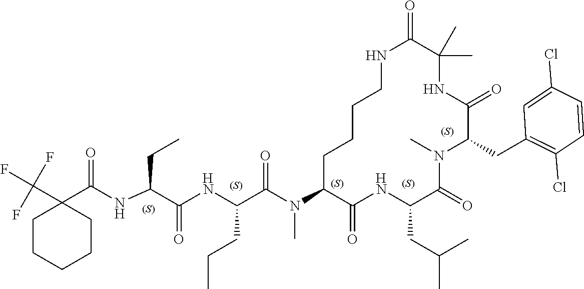 |
| 178 | 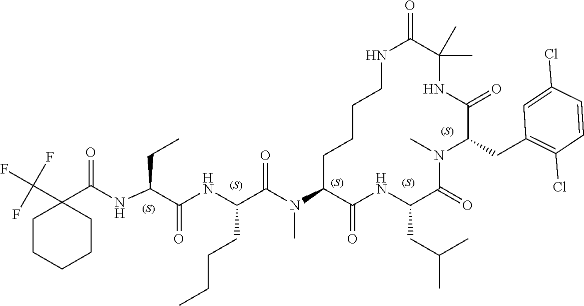 |
| 179 | 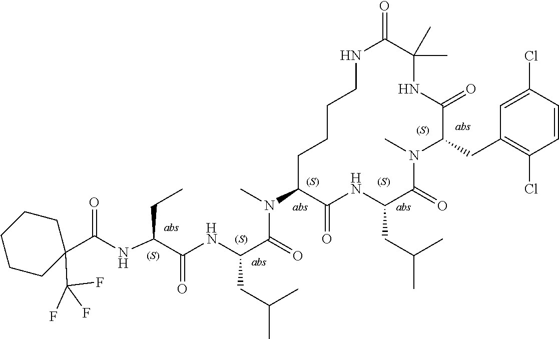 |
| 180 | 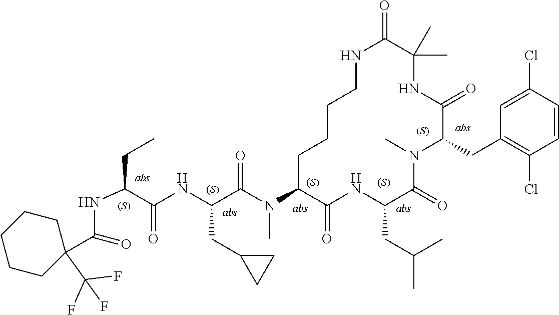 |
| 181 | 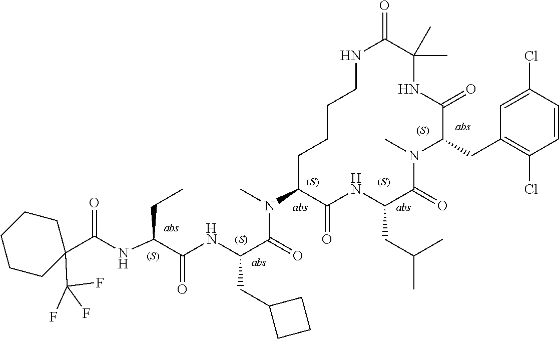 |
| 182 | 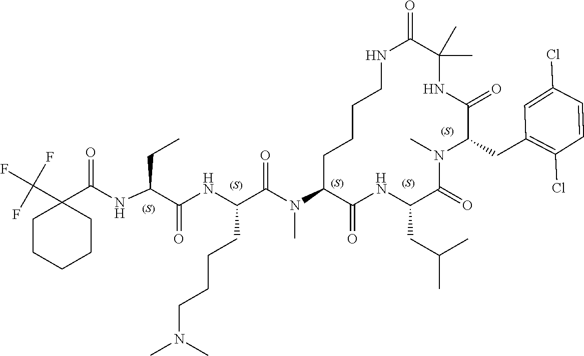 |
| 183 | 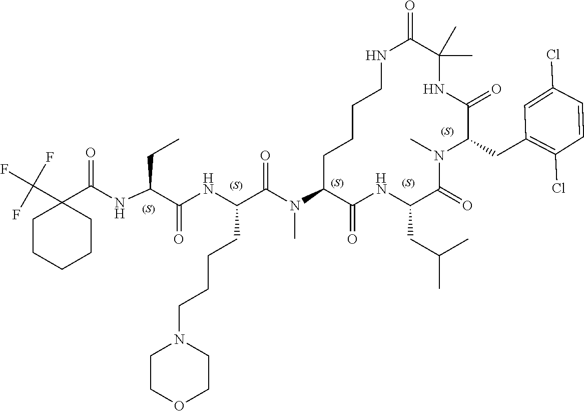 |
| 184 | 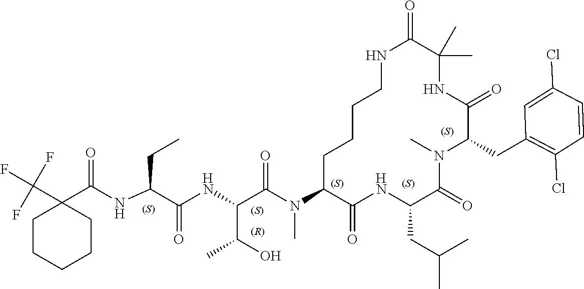 |
| 185 | 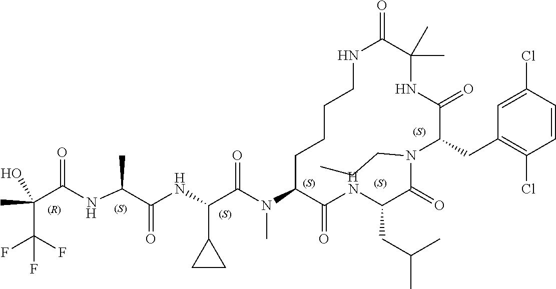 |
| 186 | 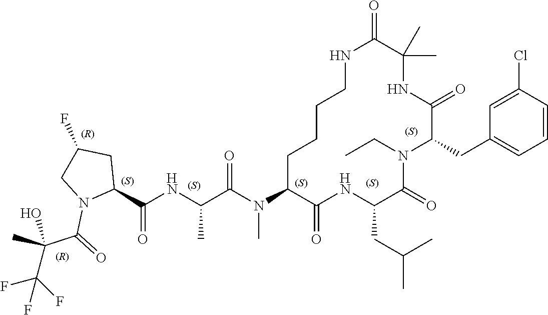 |
| 187 | 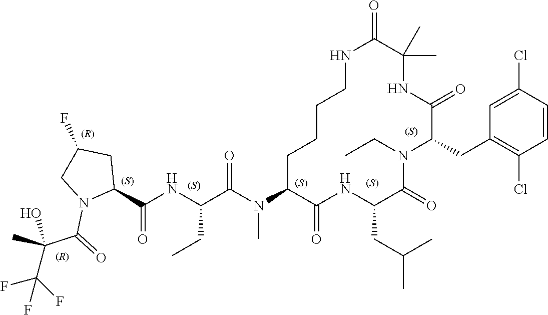 |
| 188 | 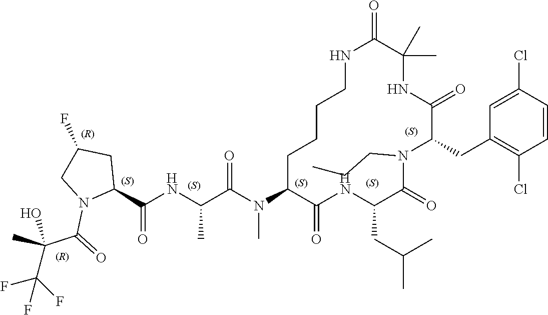 |
| 189 | 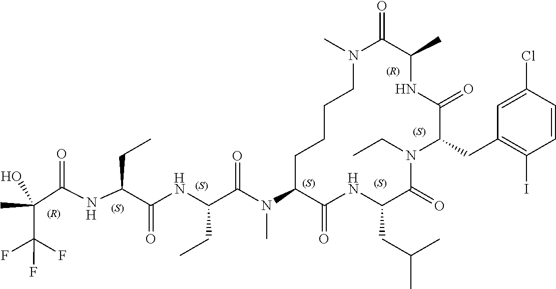 |
| 190 | 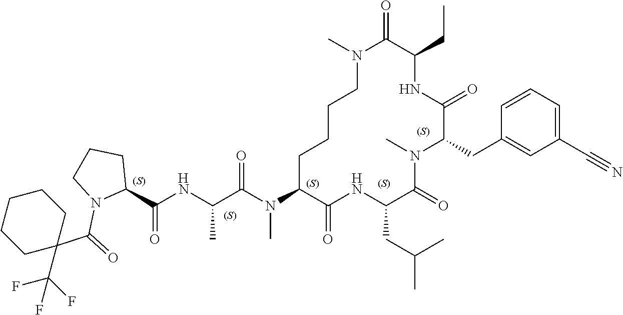 |
| 191 | 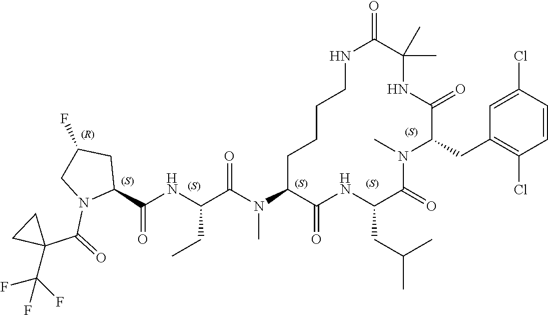 |
| 192 | 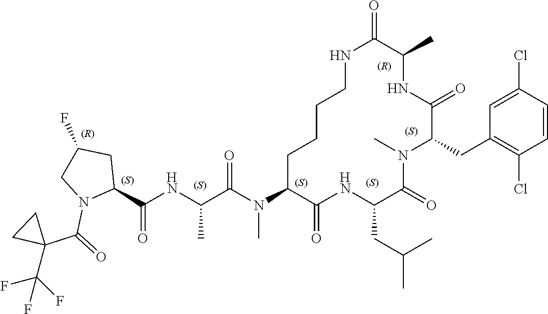 |
| 193 | 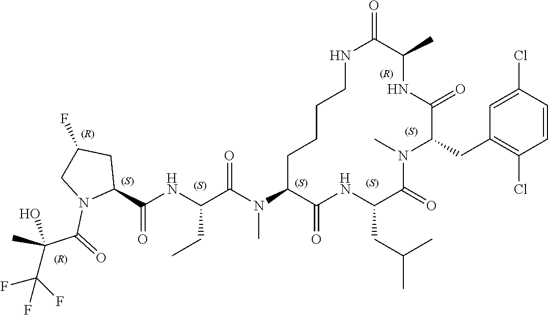 |
| 194 | 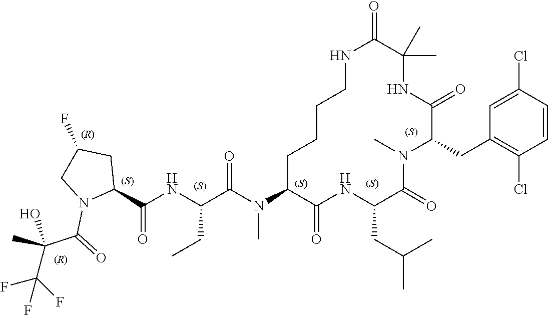 |
| 195 | 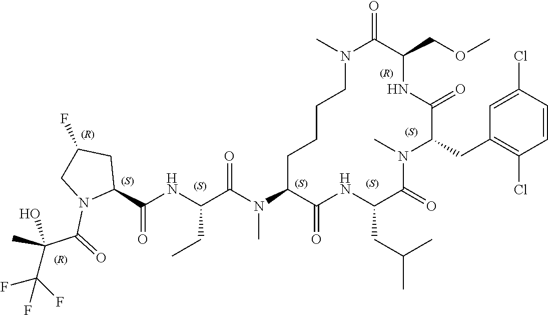 |
| 196 | 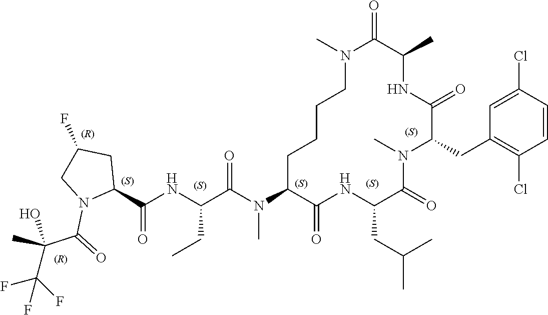 |
| 197 | 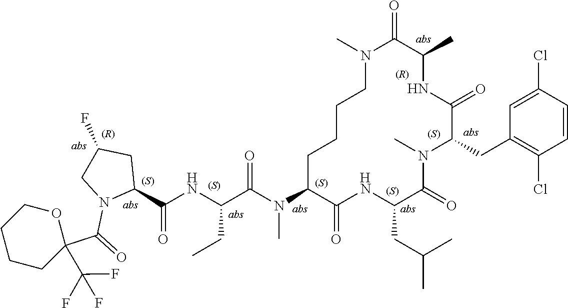 |
| 198 | 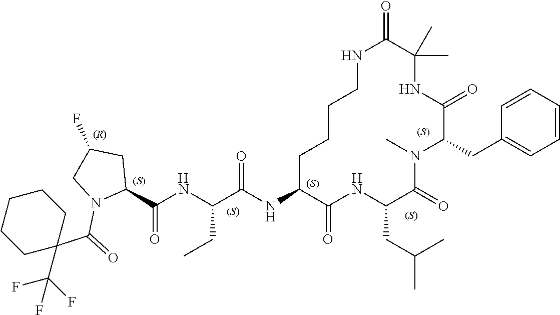 |
| 199 | 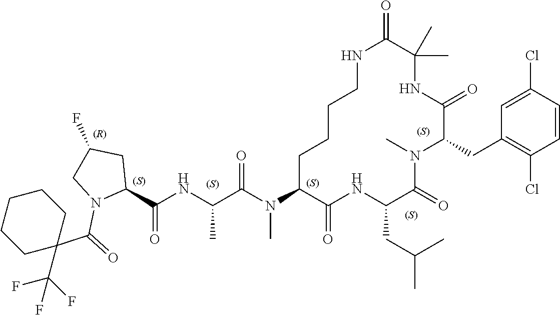 |
| 200 | 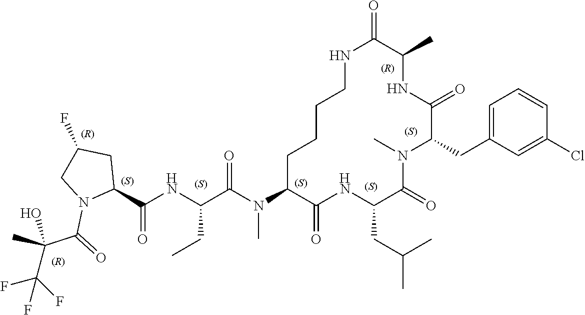 |
| 201 | 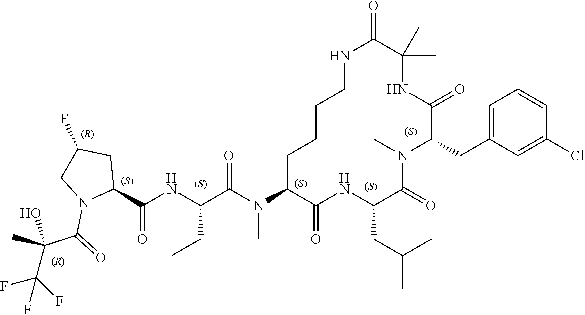 |
| 202 | 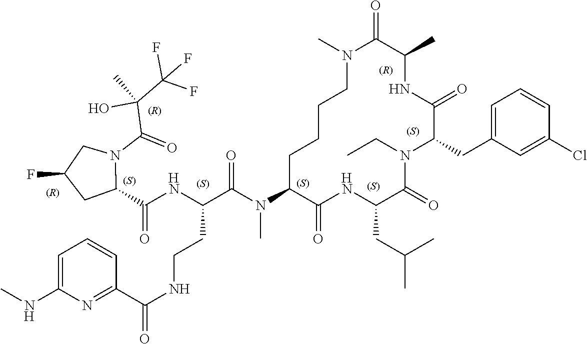 |
| 203 | 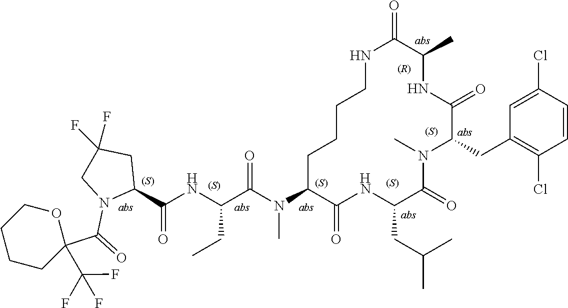 |
| 204 | 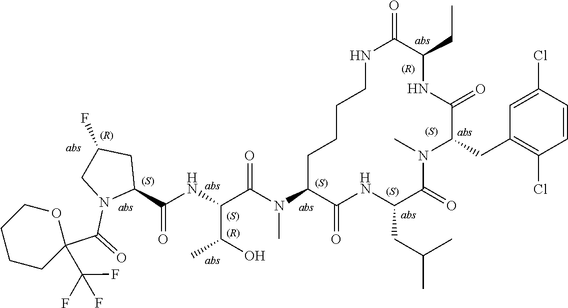 |
| 205 | 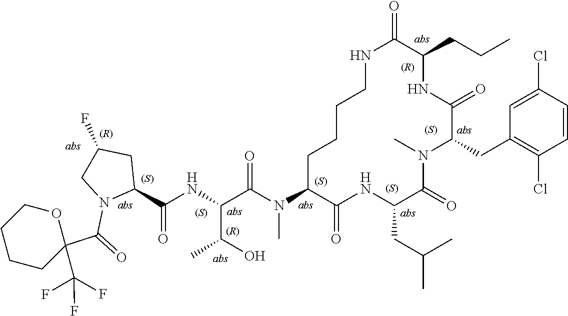 |
| 206 | 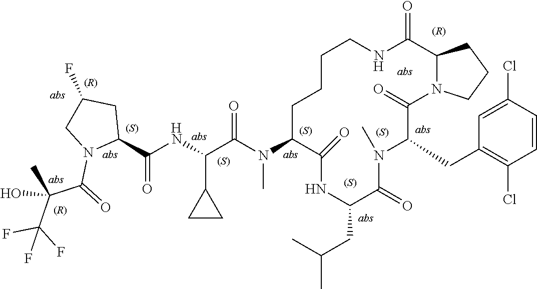 |
| 207 | 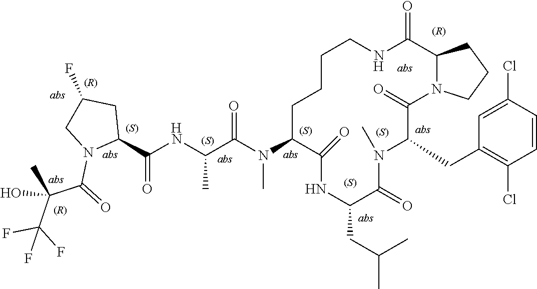 |
| 208 | 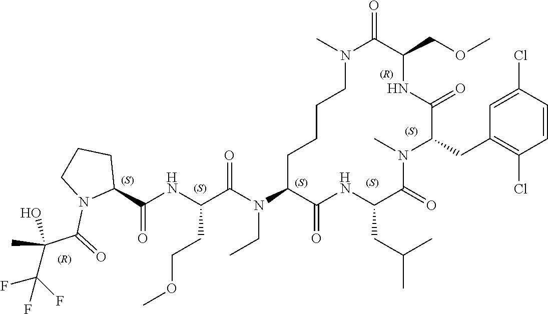 |
| 209 | 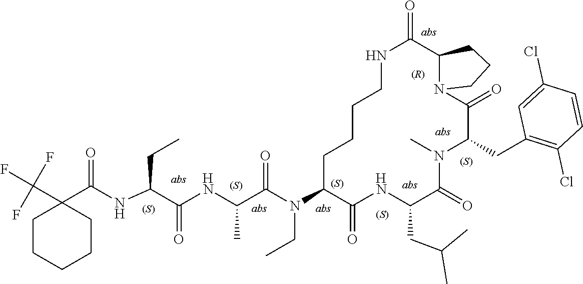 |
| 210 | 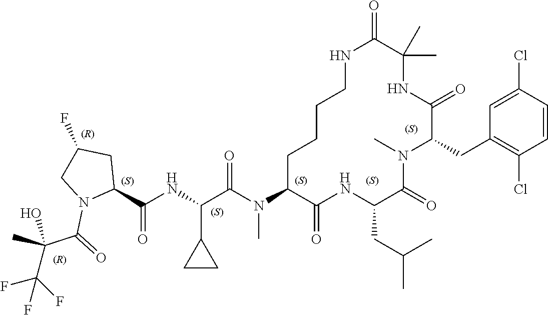 |
| 211 | 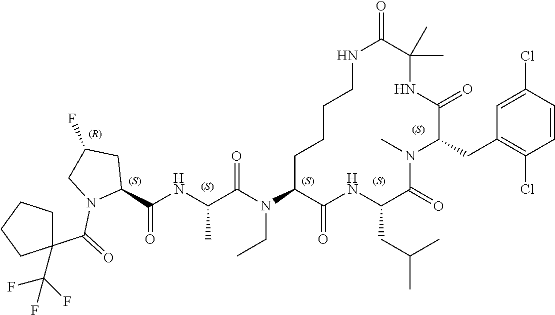 |
| 212 | 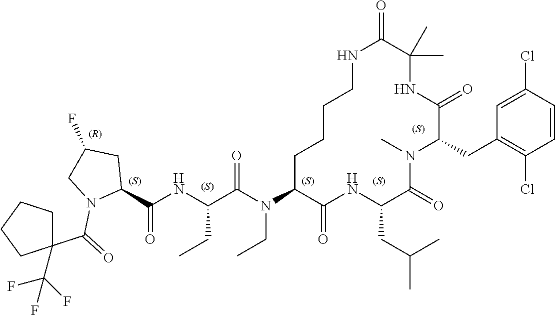 |
| 213 | 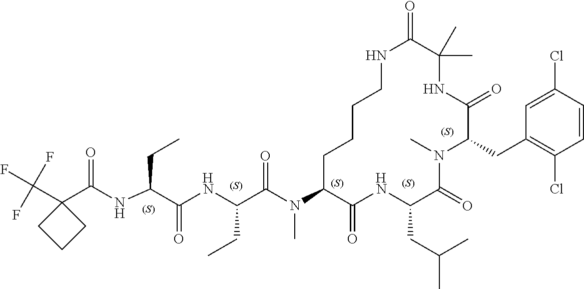 |
| 214 | 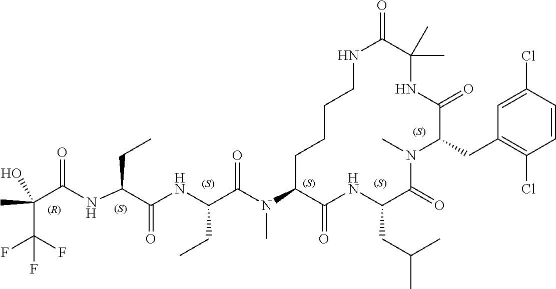 |
| 215 | 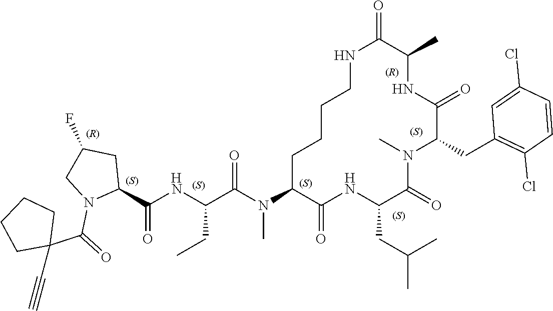 |
| 216 | 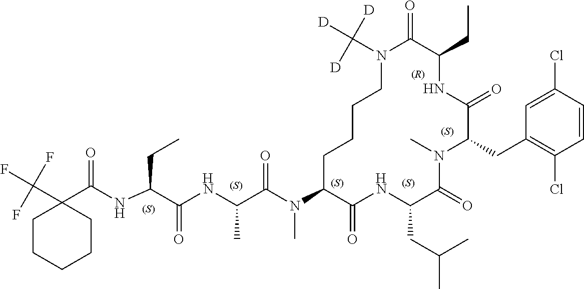 |
| 217 | 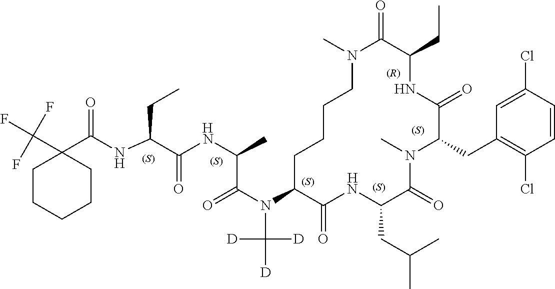 |
| 218 | 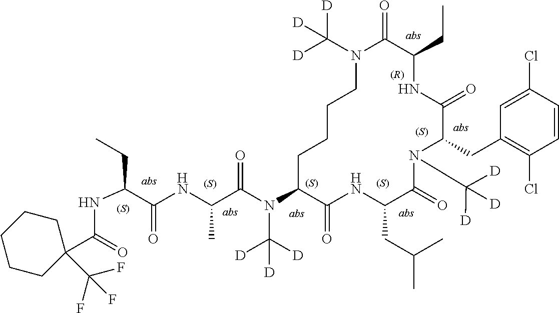 |
| 219 | 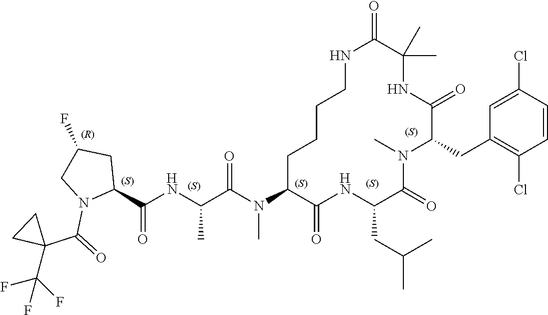 |
| 220 | 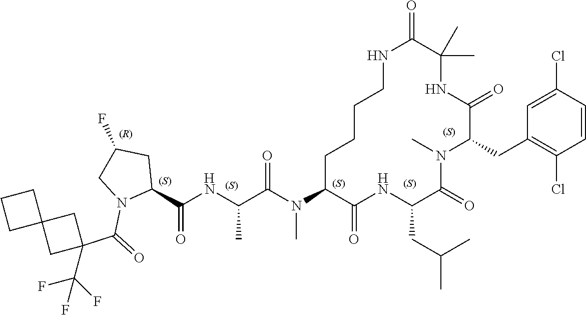 |
| 221 | 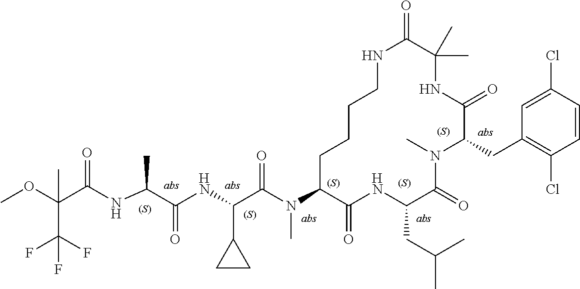 |
| 222 | 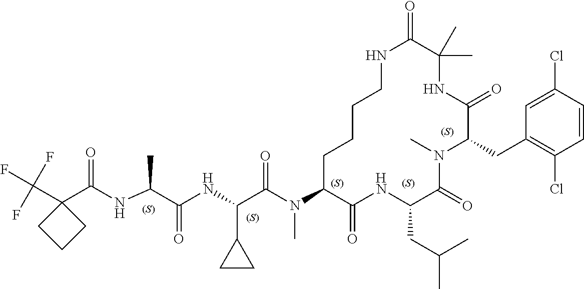 |
| 223 | 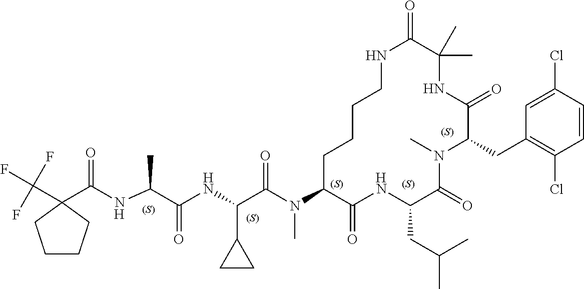 |
| 224 | 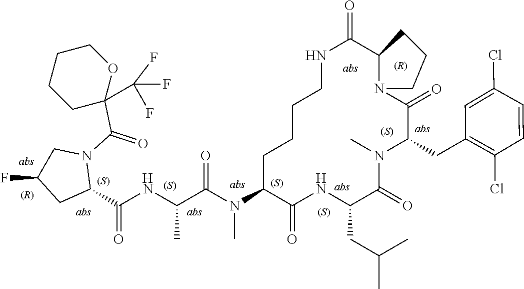 |
| 225 | 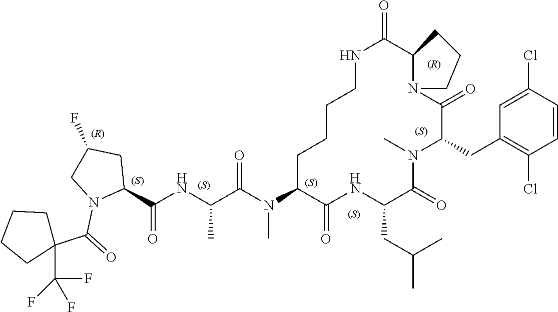 |
| 226 | 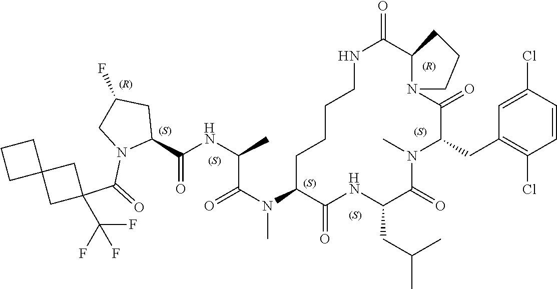 |
| 227 | 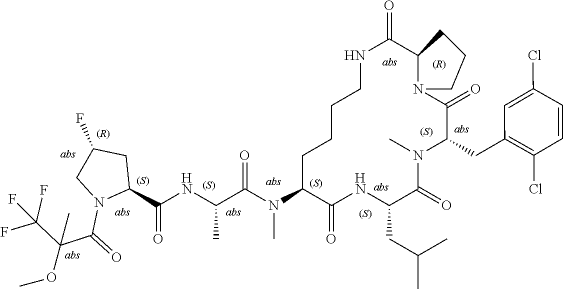 |
| 228 | 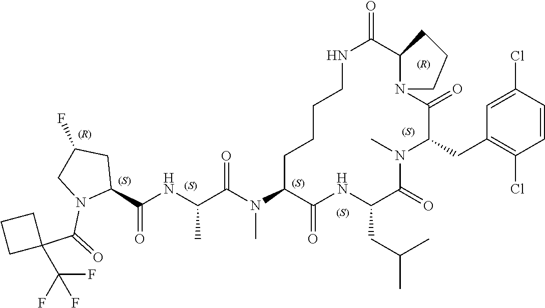 |
| 229 | 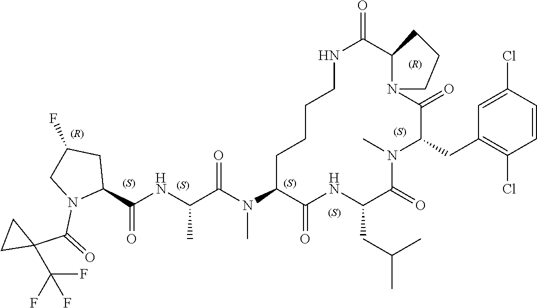 |
| 230 | 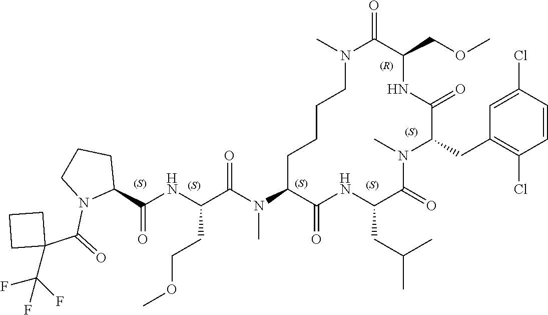 |
| 231 | 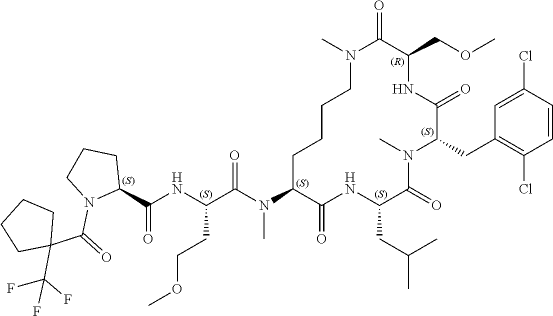 |
| 232 | 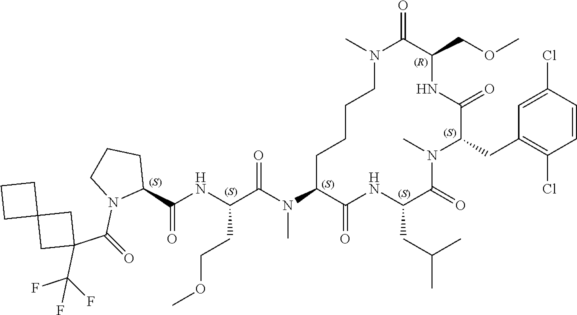 |
| 233 | 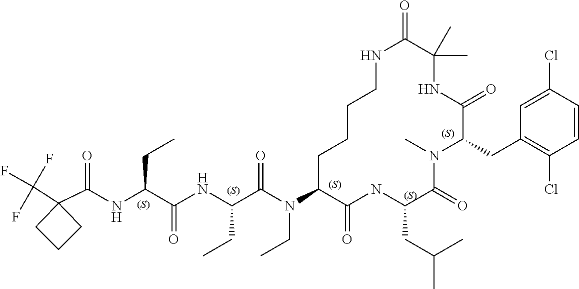 |
| 234 | 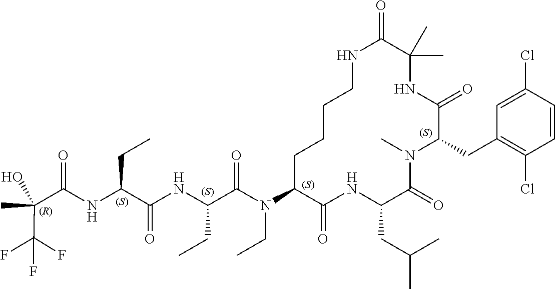 |
| 235 | 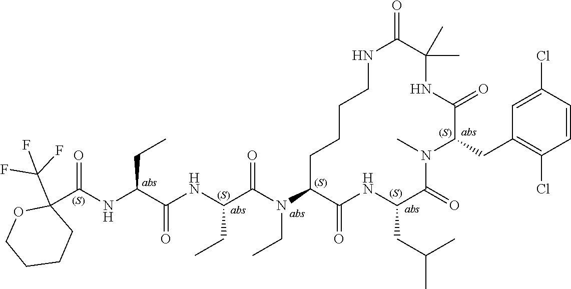 |
| 236 | 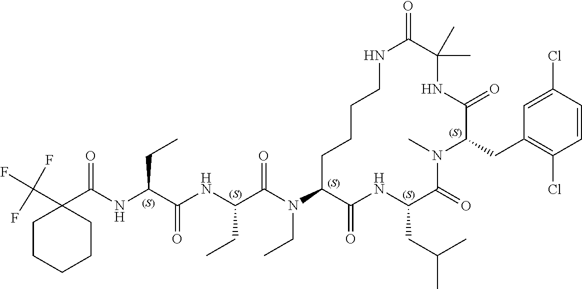 |
| 237 | 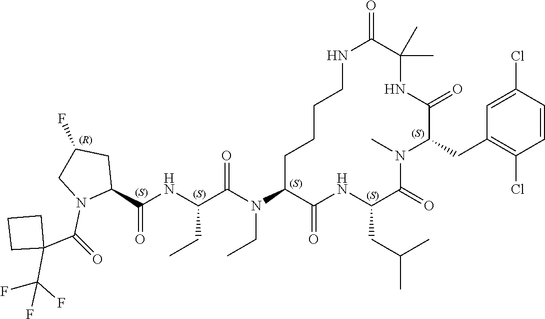 |
| 238 | 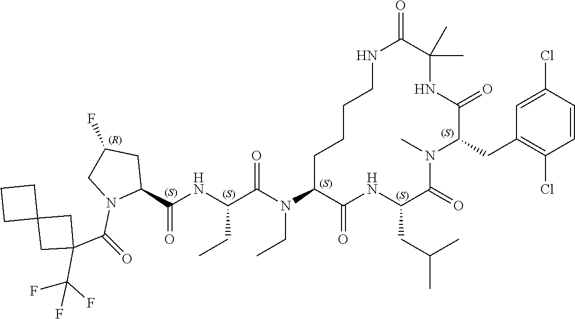 |
| 239 | 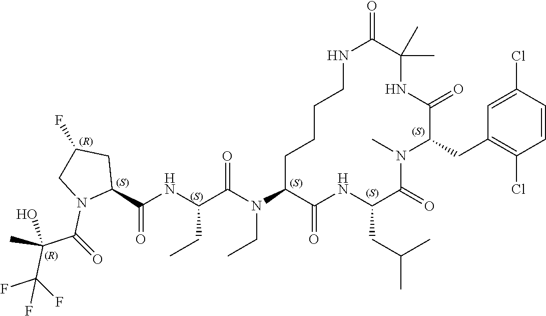 |
| 240 | 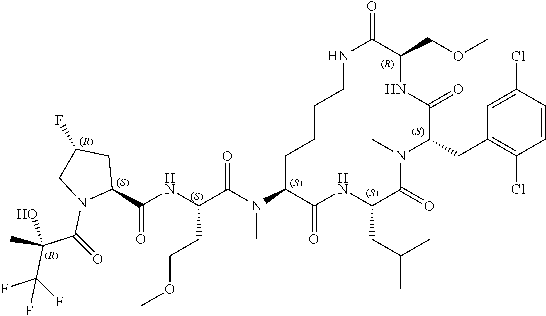 |
| 241 | 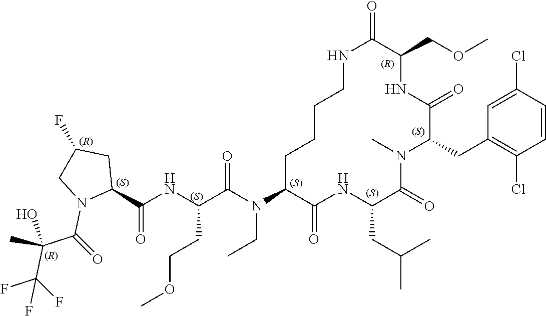 |
| 242 | 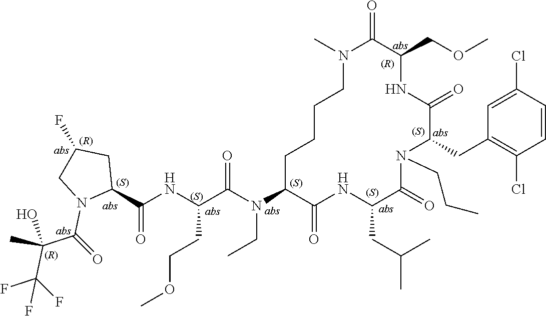 |
| 243 | 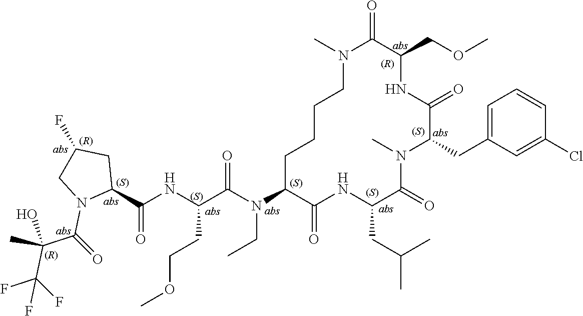 |
| 244 | 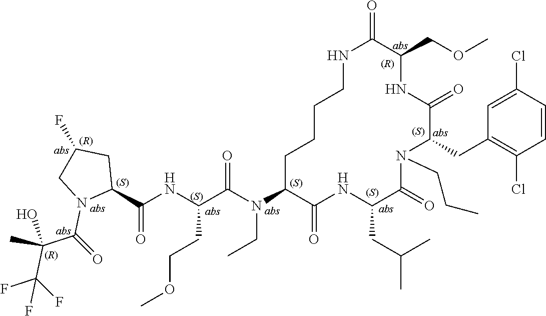 |
| 245 | 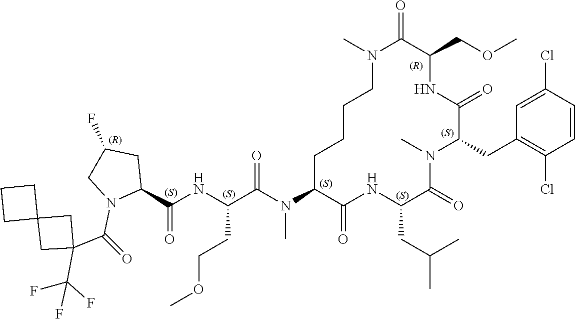 |
| 246 | 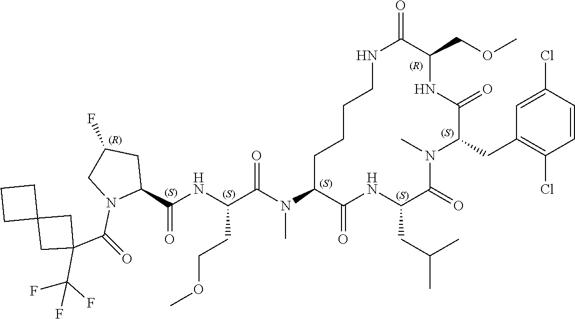 |
| 247 | 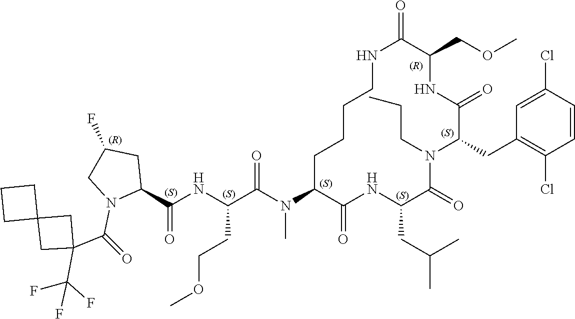 |
| 248 | 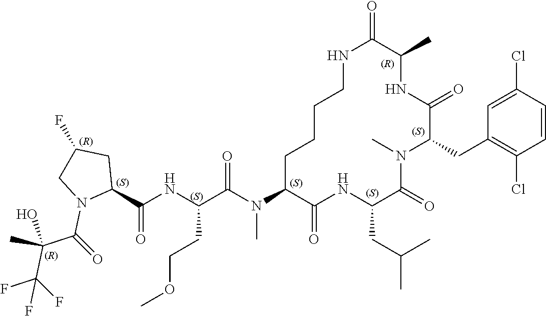 |
| 249 | 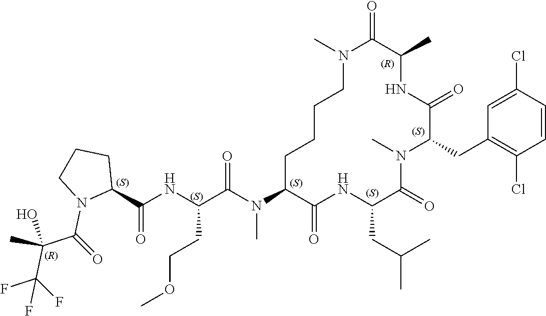 |
| 250 | 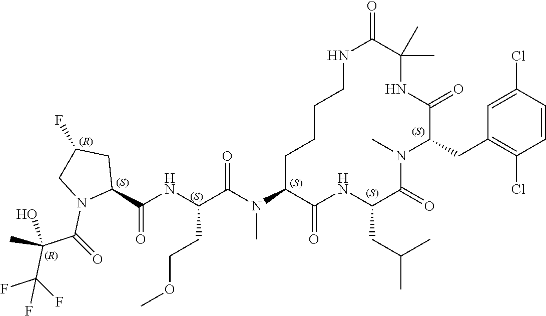 |
| 251 | 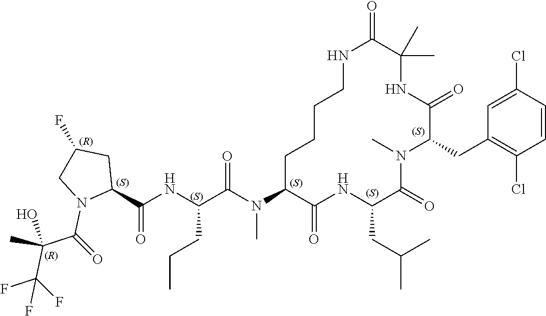 |
| 252 | 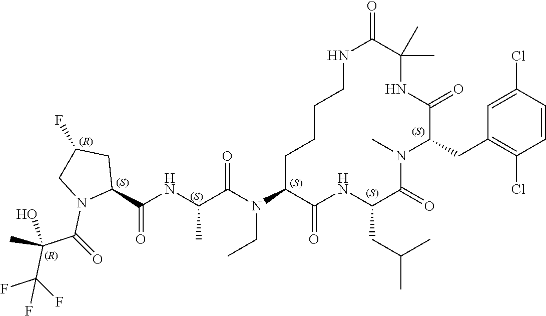 |
| 253 | 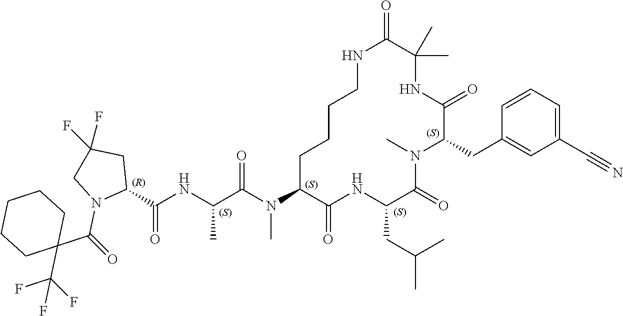 |
| 254 | 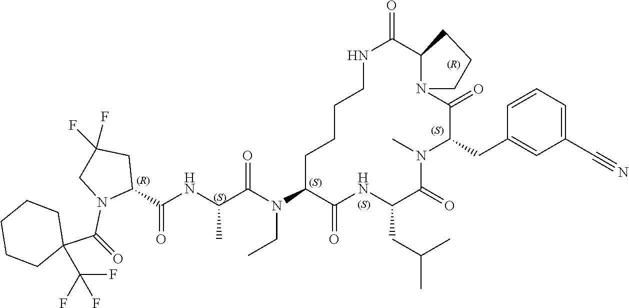 |
| 255 | 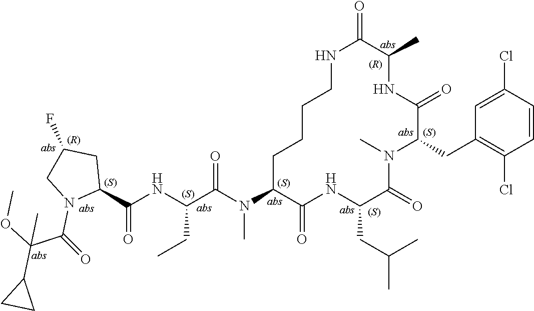 |
| 256 | 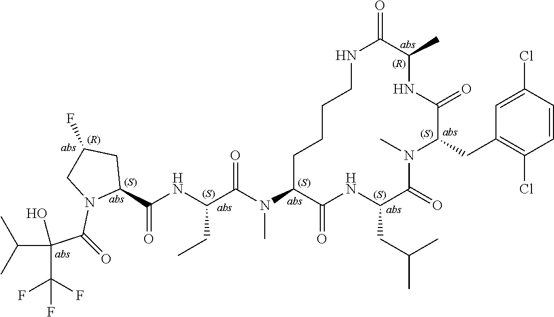 |
| 257 | 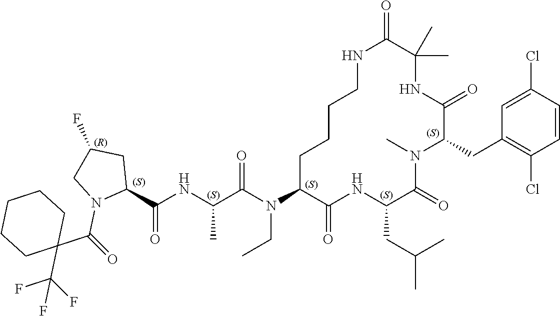 |
| 258 | 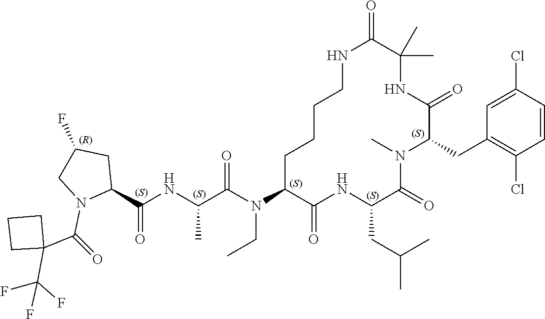 |
| 259 | 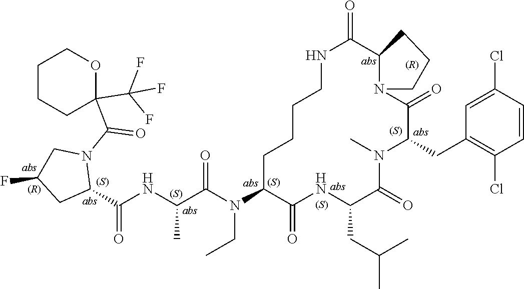 |
| 260 | 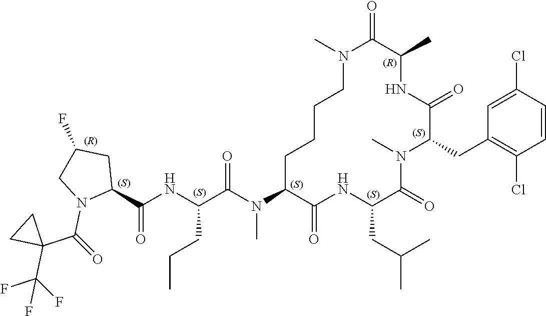 |
| 261 | 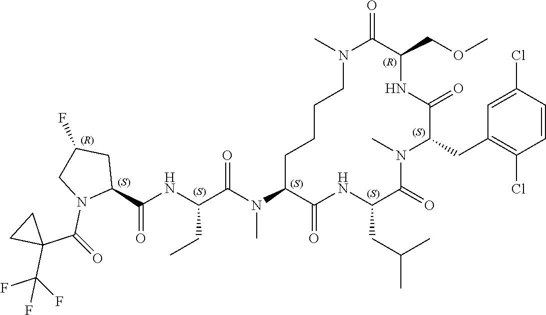 |
| 262 | 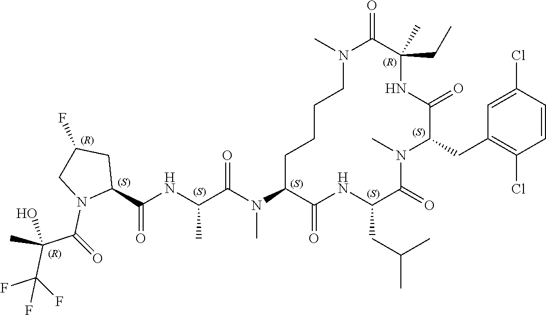 |
| 263 | 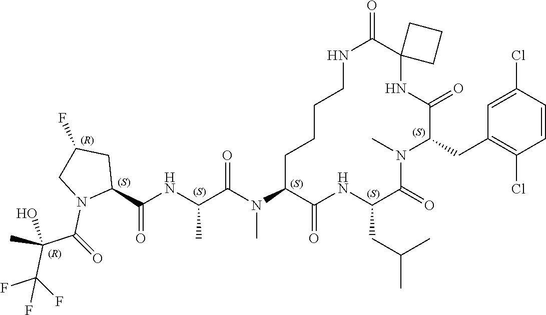 |
| 264 | 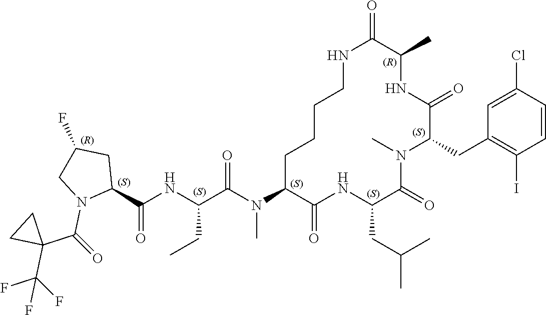 |
| 265 | 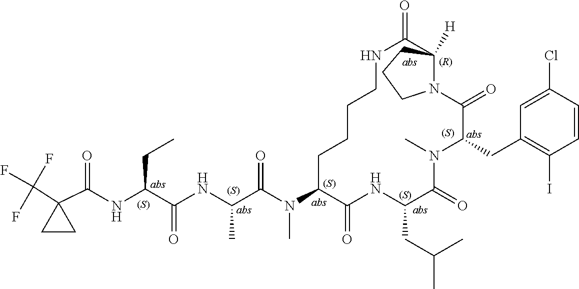 |
| 266 | 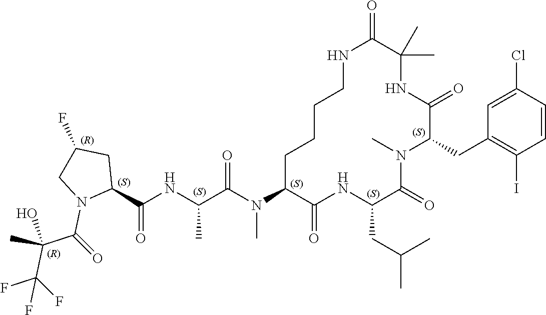 |
| 267 | 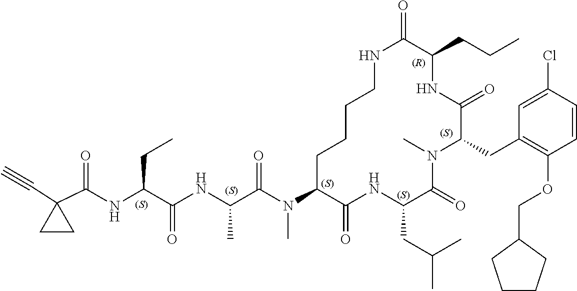 |
| 268 | 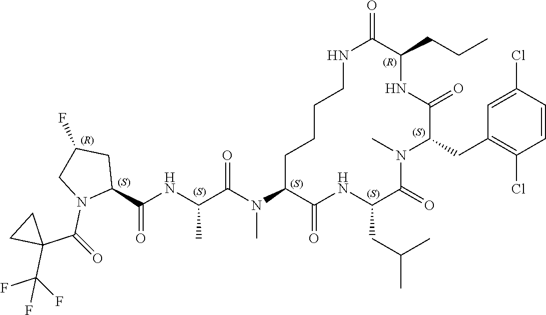 |
| 269 | 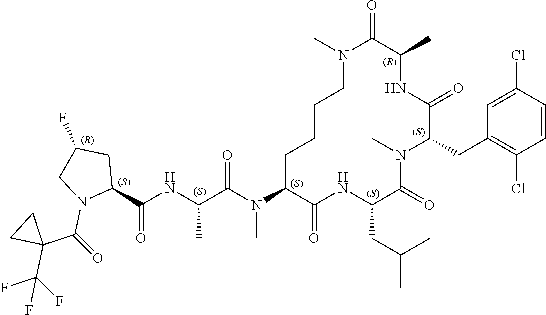 |
| 270 | 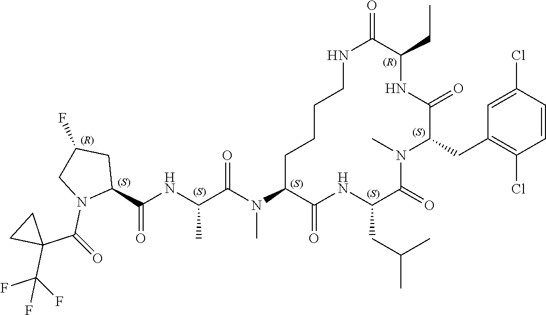 |
| 271 | 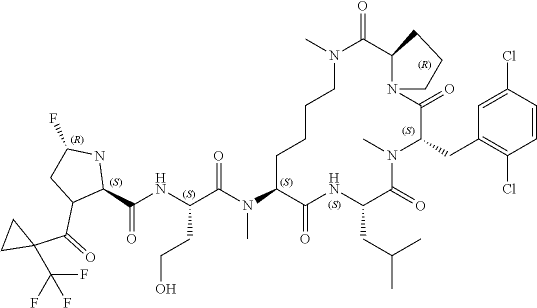 |
| 272 | 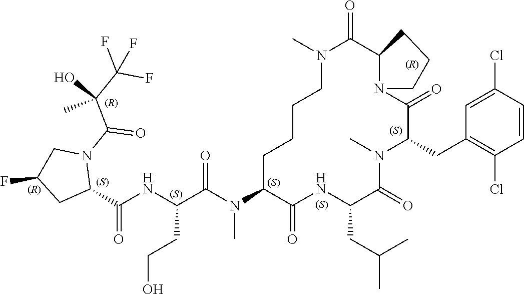 |
| 273 | 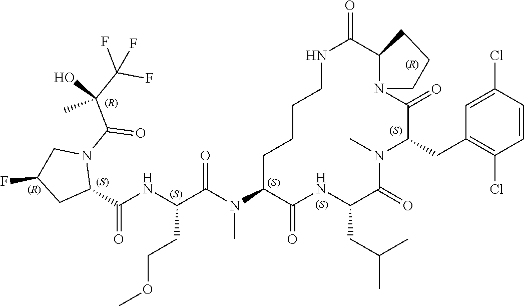 |
| 274 | 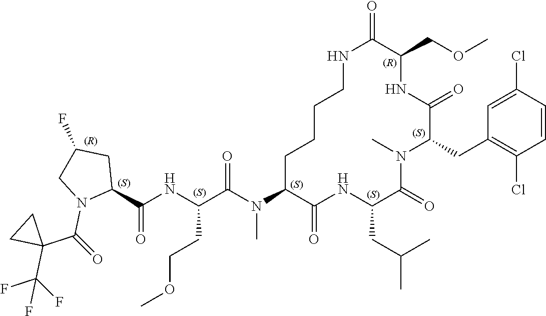 |
| 275 | 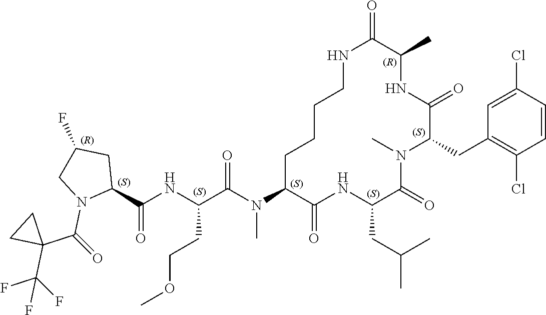 |
| 276 | 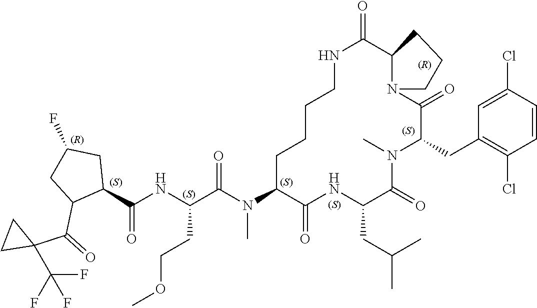 |
| 277 | 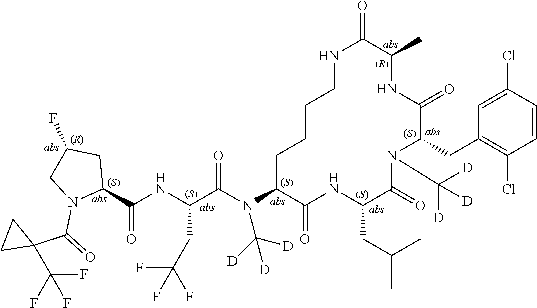 |
| 278 | 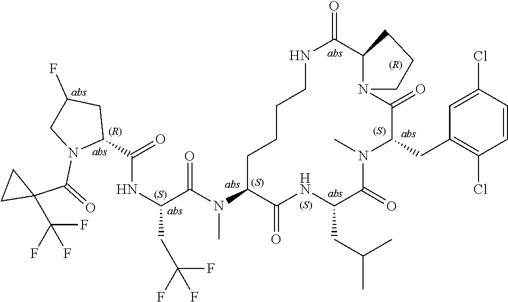 |
| 279 | 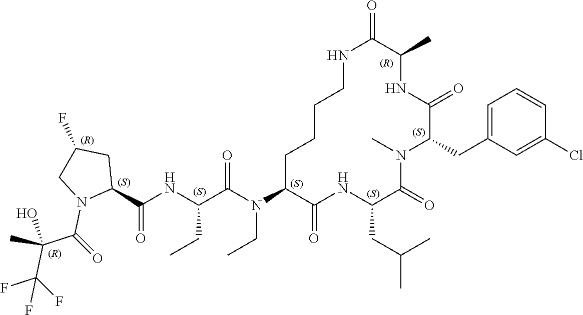 |
| 280 | 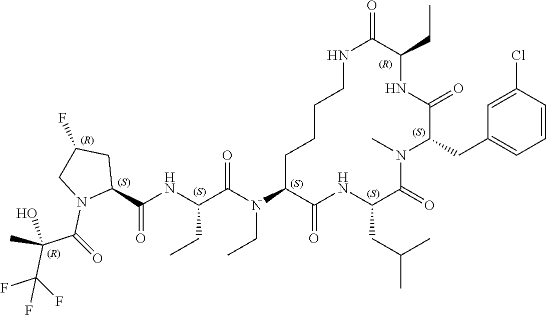 |
| 281 | 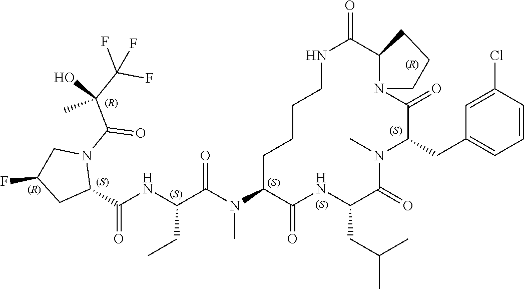 |
| 282 | 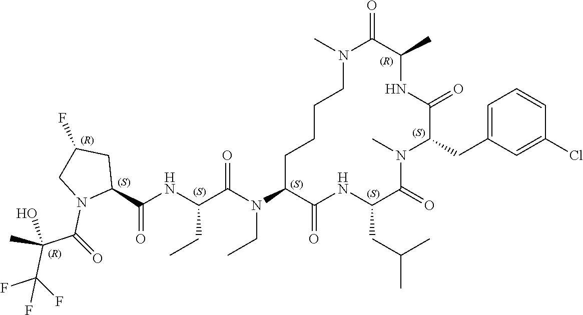 |
| 283 | 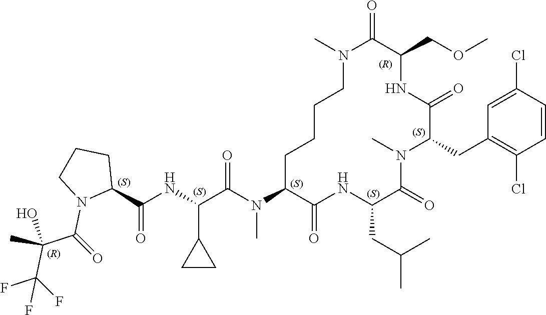 |
| 284 | 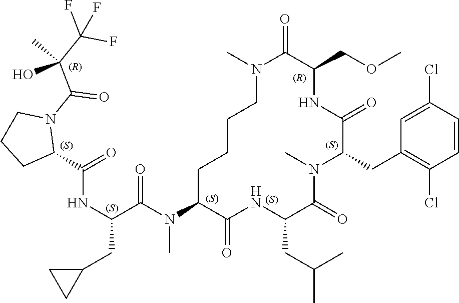 |
| 285 | 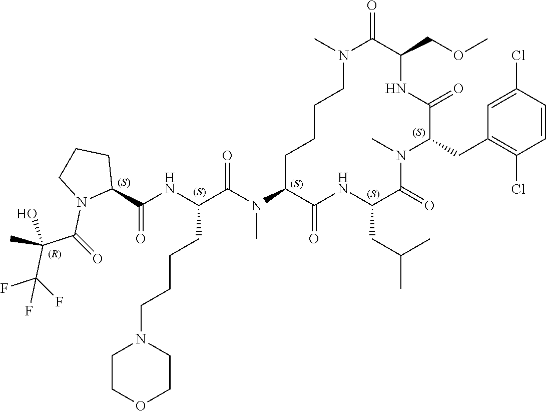 |
| 286 | 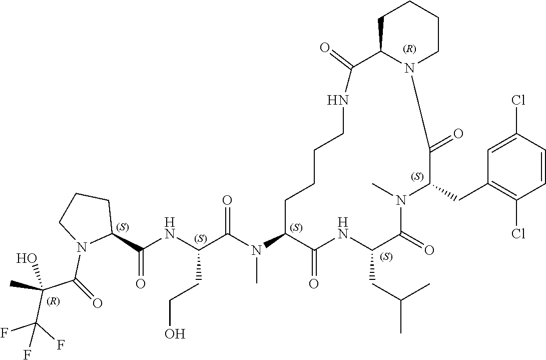 |
| 287 | 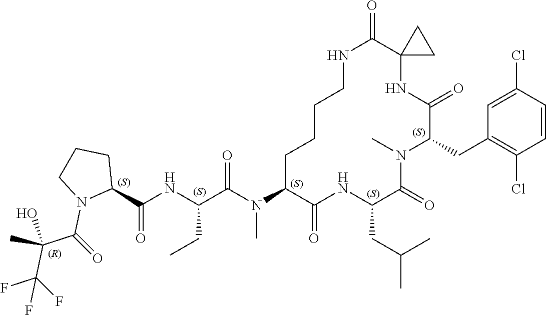 |
| 288 | 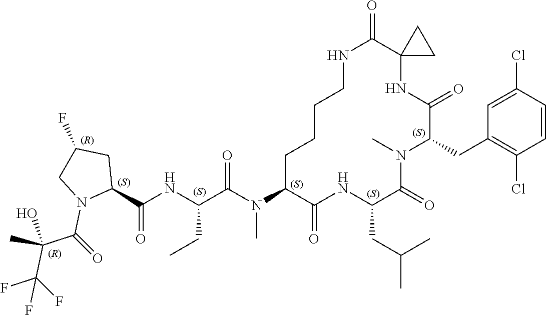 |
| 289 | 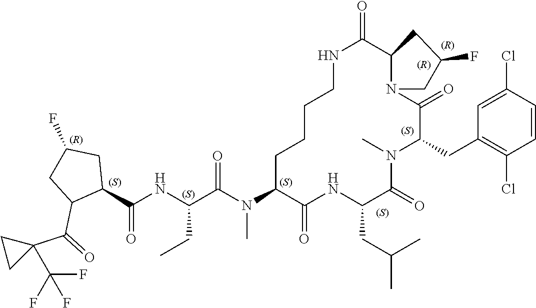 |
| 290 | 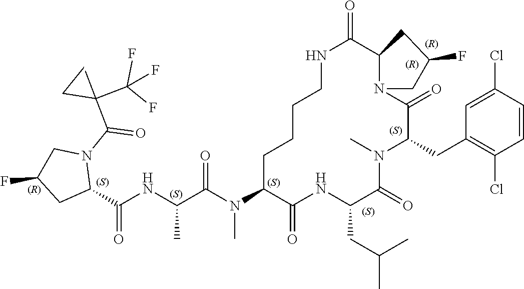 |
| 291 | 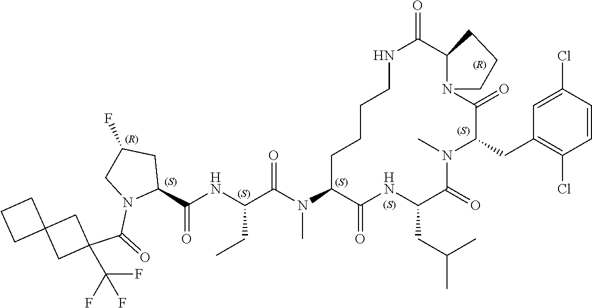 |
| 292 | 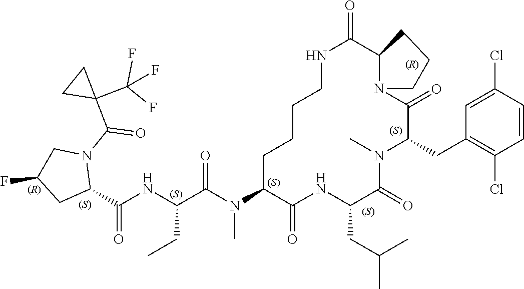 |
| 293 | 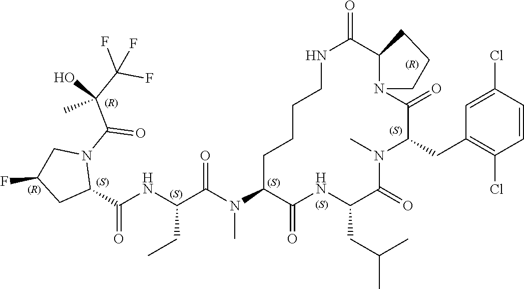 |
| 294 | 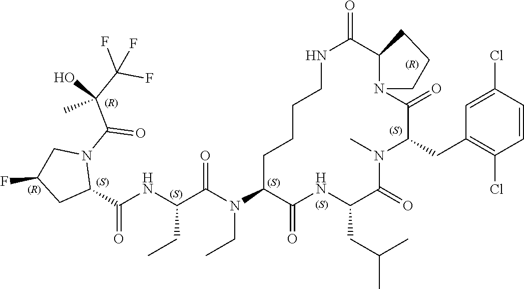 |
| 295 | 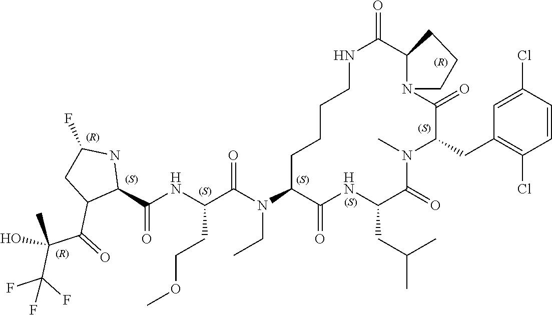 |
| 296 | 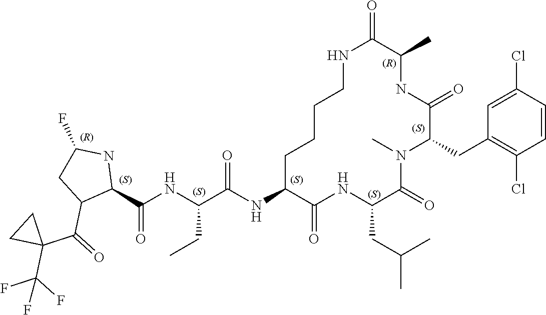 |
| 297 | 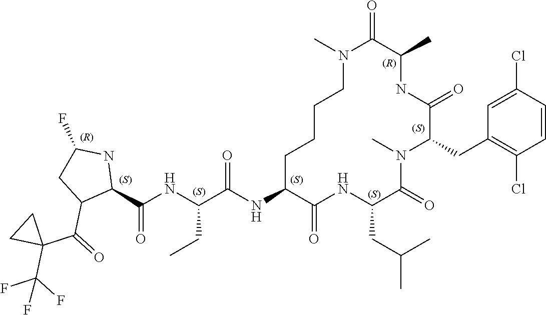 |
| 298 | 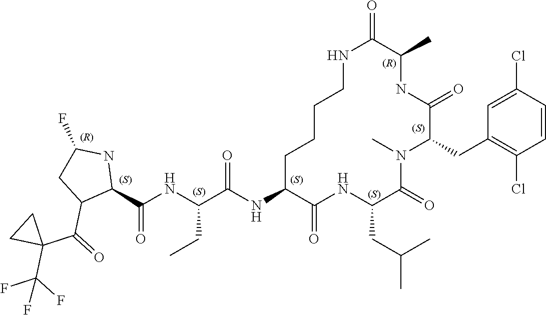 |
| 299 | 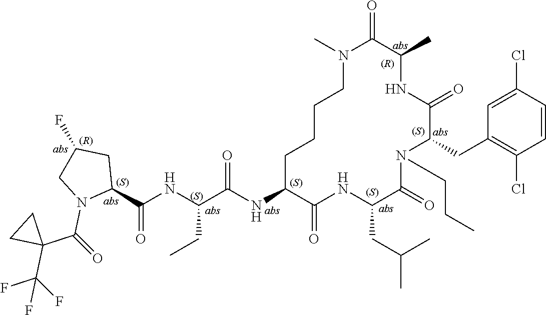 |
| 300 | 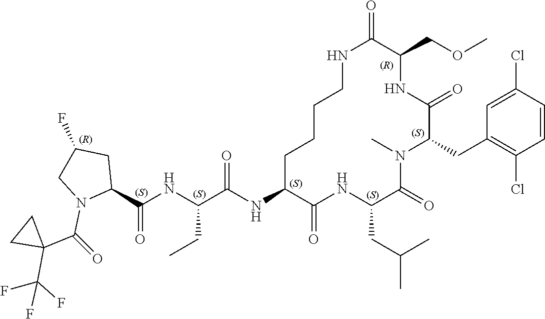 |
| 301 | 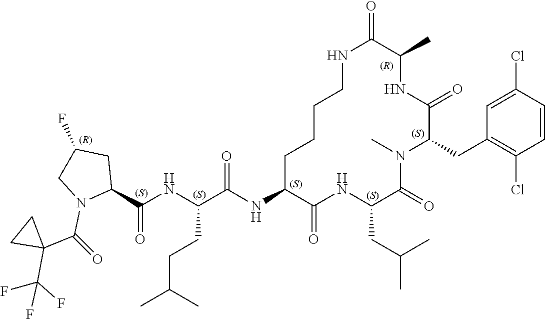 |
| 302 | 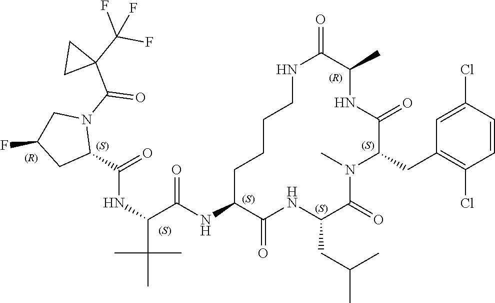 |
| 303 | 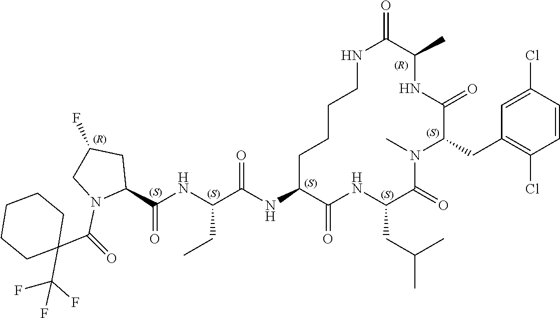 |
| 304 | 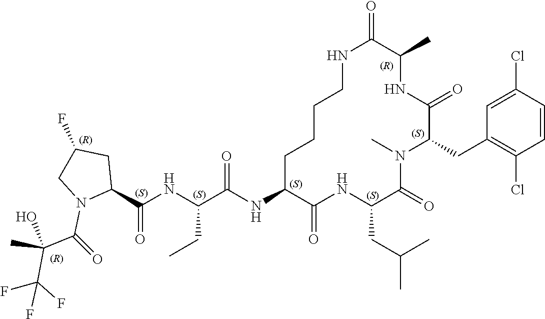 |
| 305 | 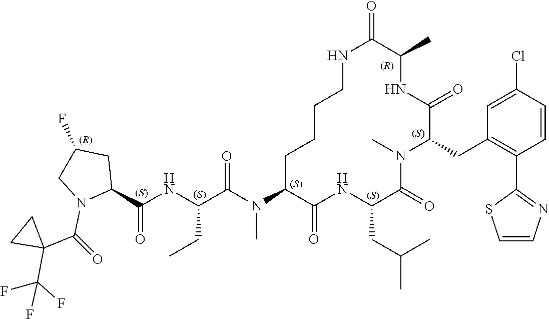 |
| 306 | 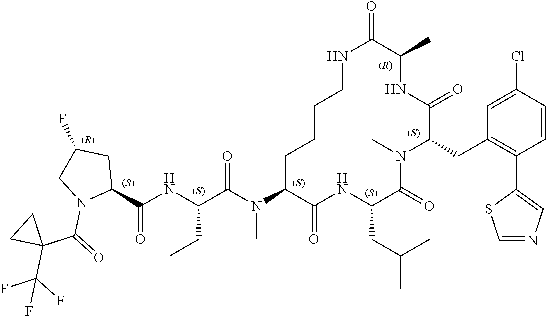 |
| 307 | 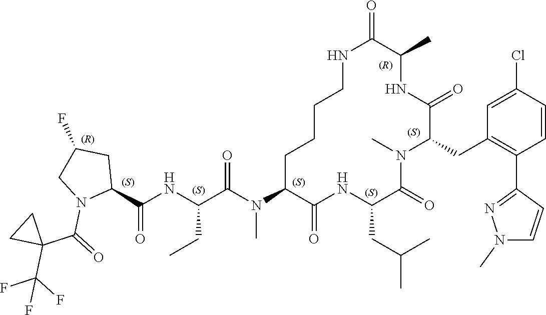 |
| 308 | 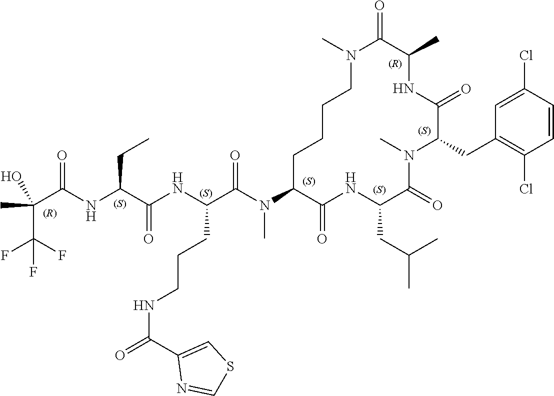 |
| 309 | 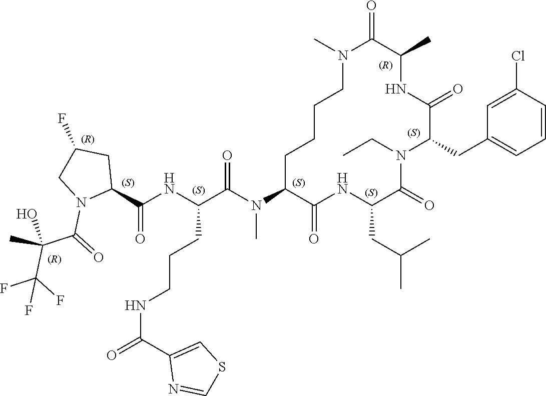 |
| 310 | 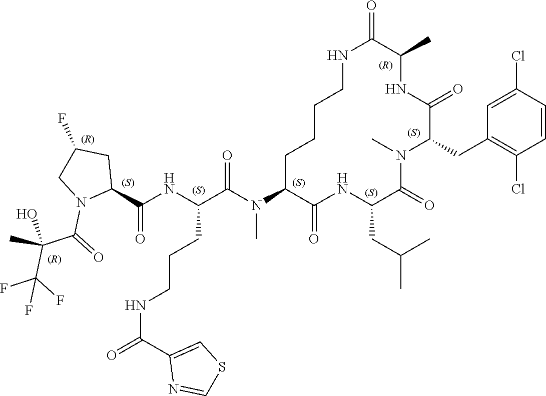 |
| 311 | 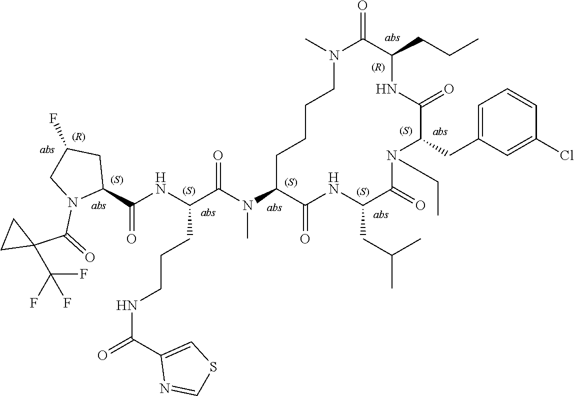 |
| 312 | 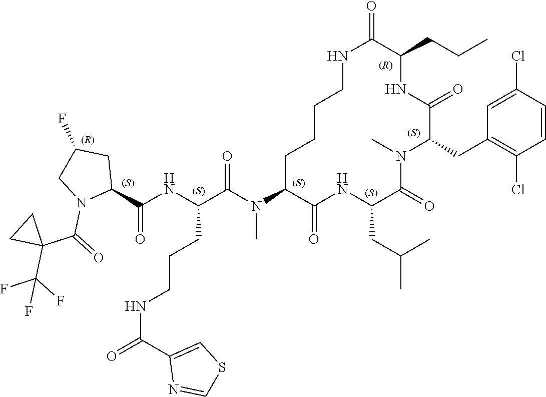 |
| 313 | 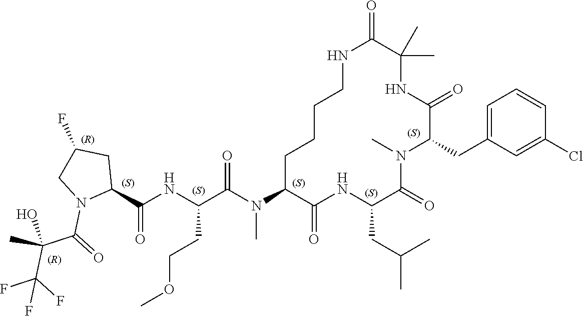 |
| 314 | 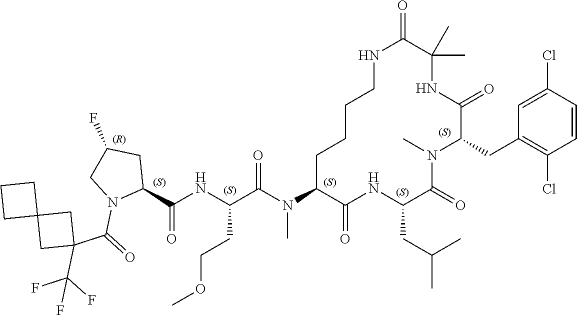 |
| 315 | 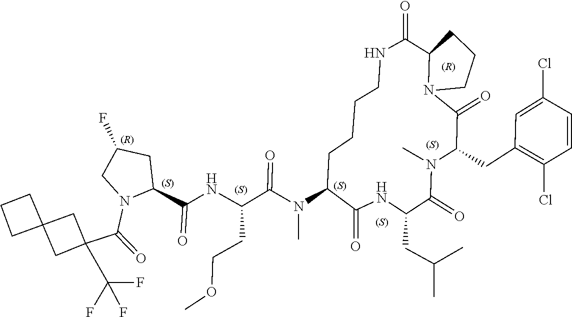 |
| 316 | 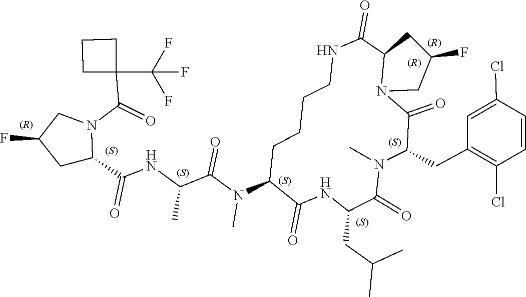 |
| 317 | 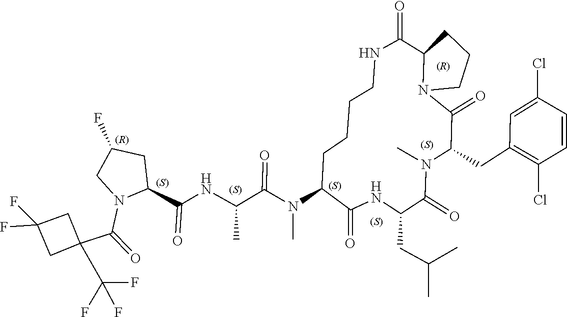 |
| 318 | 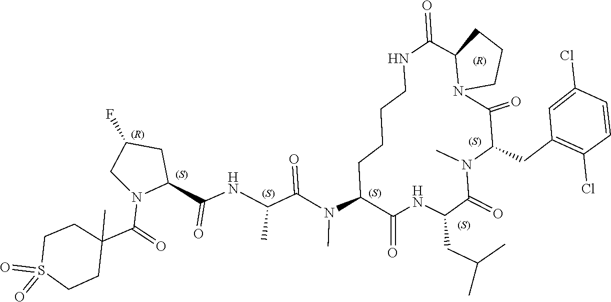 |
| 319 | 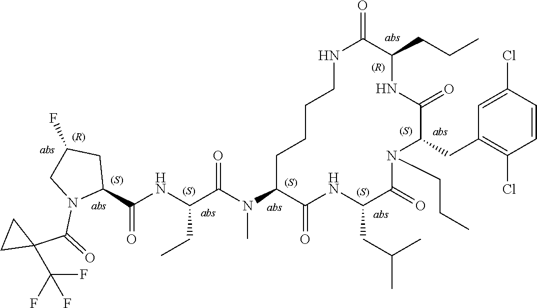 |
| 320 | 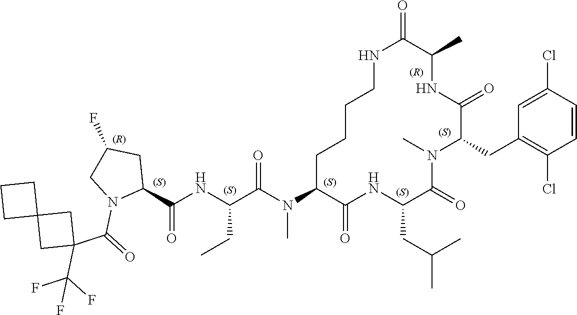 |
| 321 | 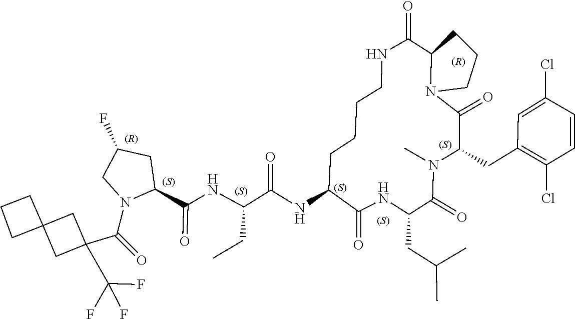 |
| 322 | 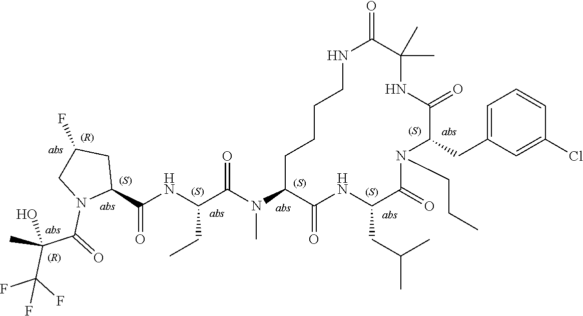 |
| 323 | 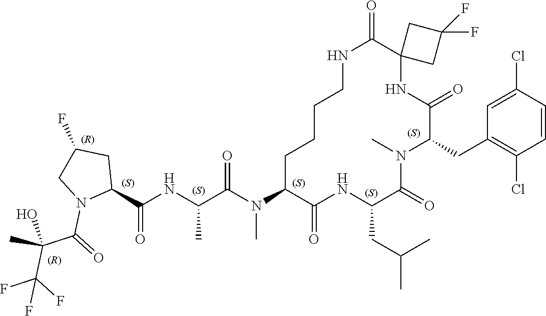 |
| 324 | 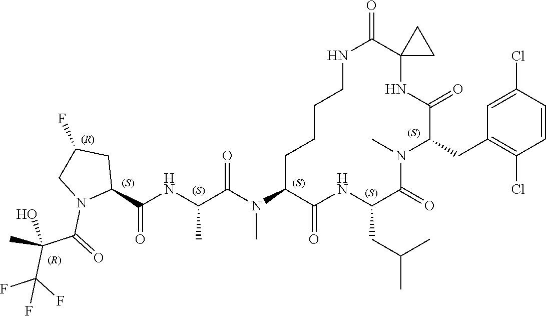 |
| 325 | 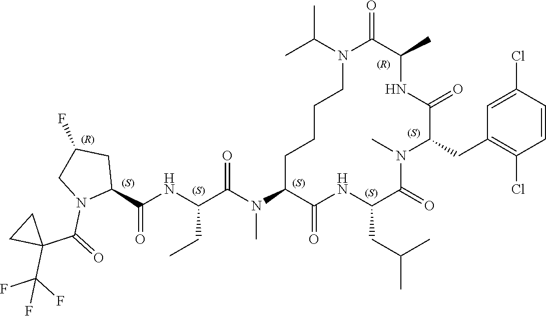 |
| 326 | 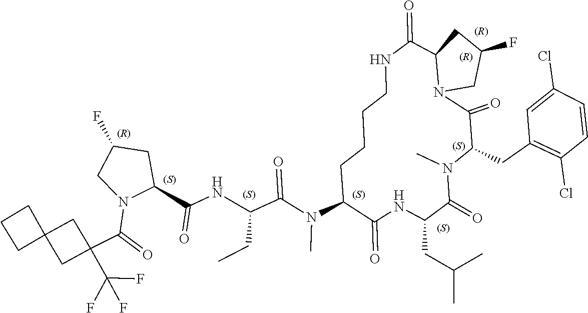 |
| 327 | 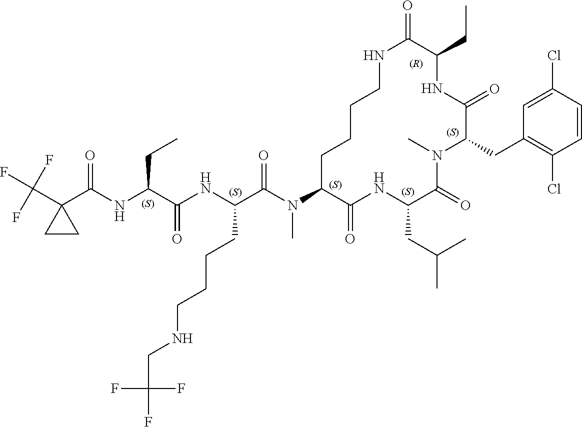 |
| 328 | 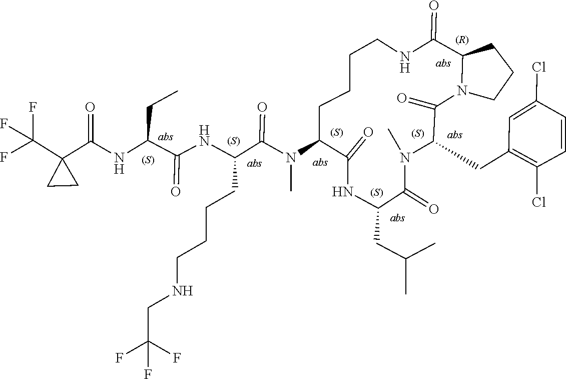 |
| 329 | 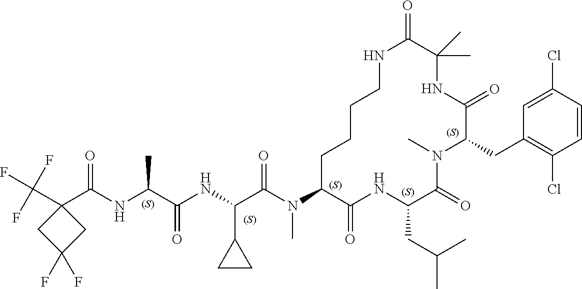 |
| 330 | 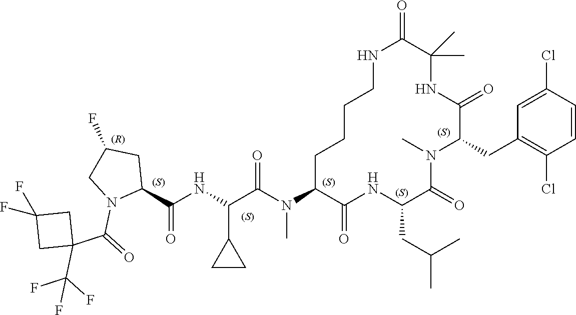 |
| 331 | 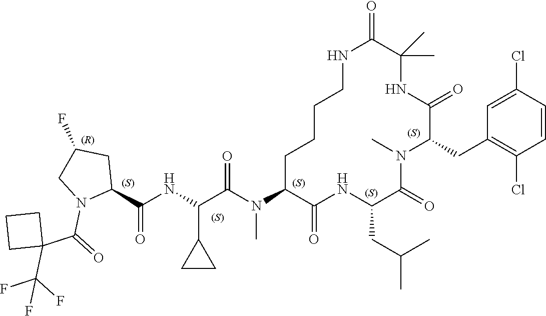 |
| 332 | 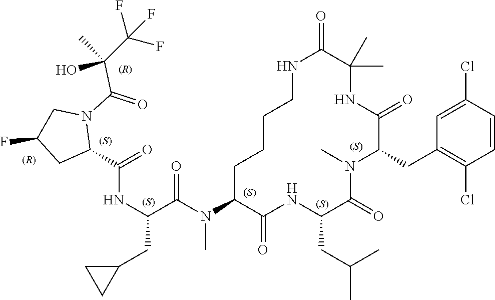 |
| 333 | 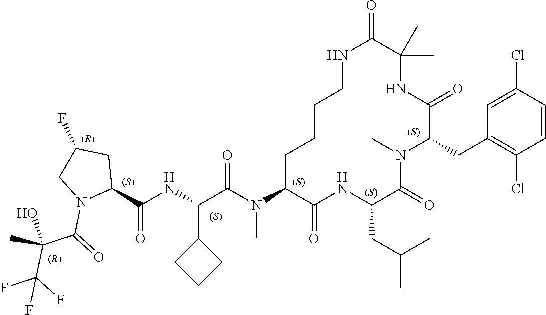 |
| 334 | 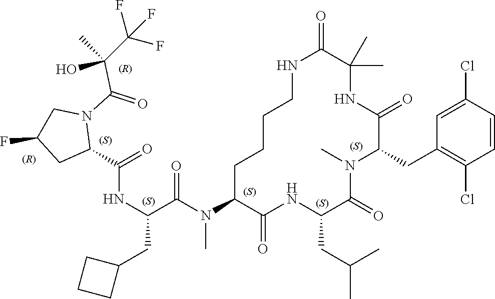 |
| 335 | 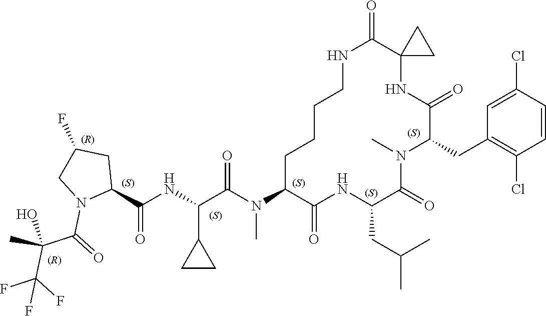 |
| 336 | 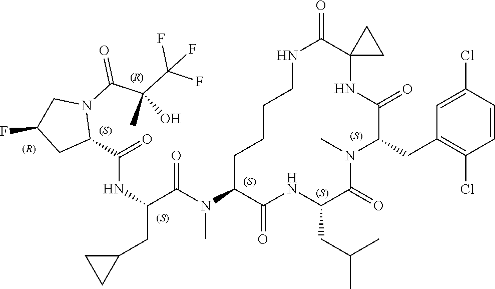 |
| 337 | 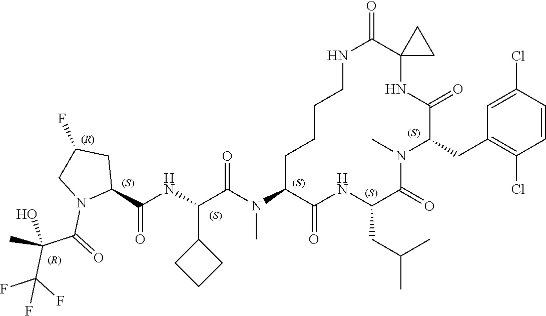 |
| 338 | 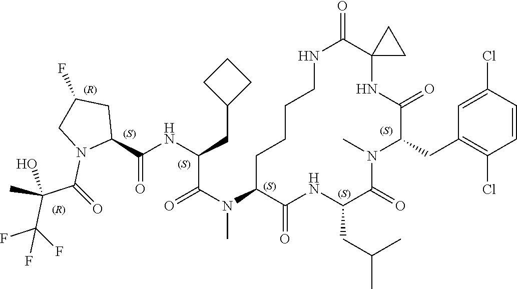 |
| 339 | 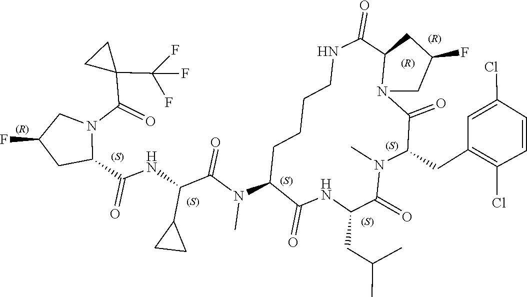 |
| 340 | 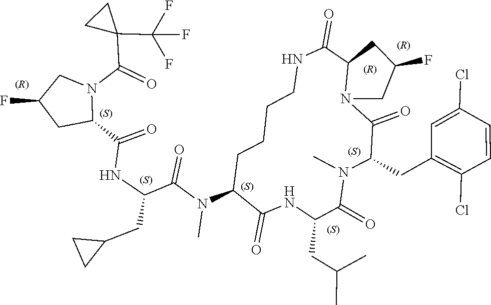 |
| 341 | 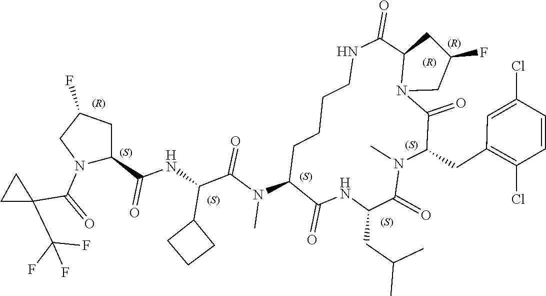 |
| 342 | 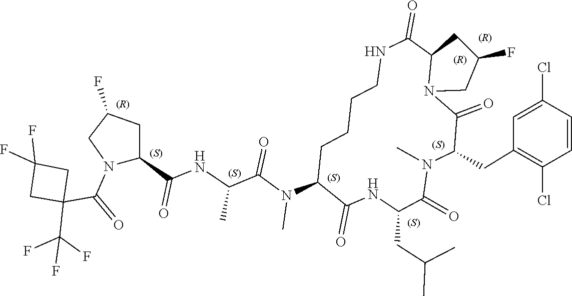 |
| 343 | 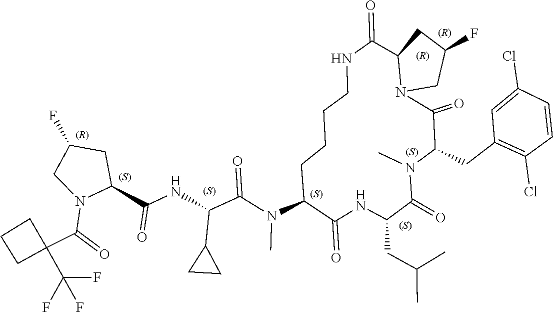 |
| 344 | 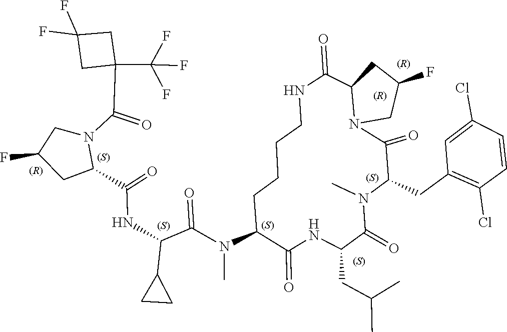 |
| 345 | 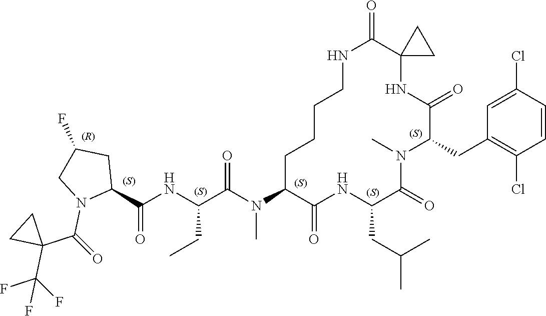 |
| 346 | 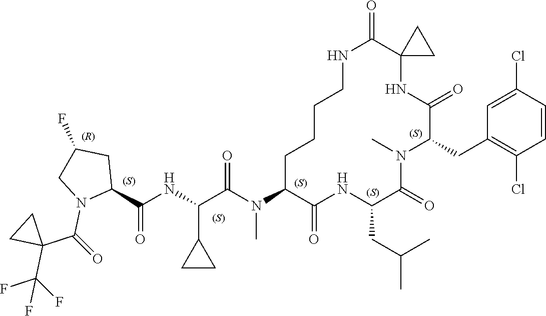 |
| 347 | 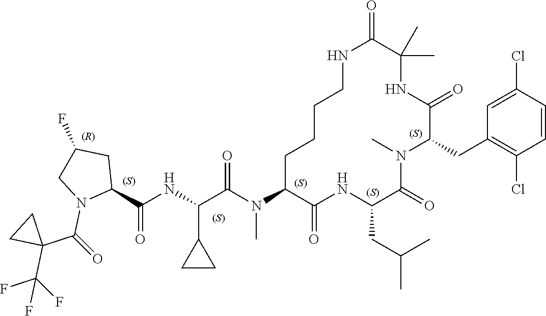 |
| 348 | 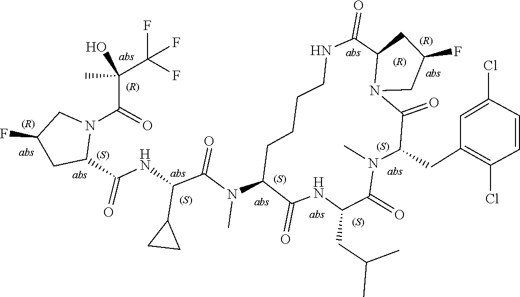 |
| 349 | 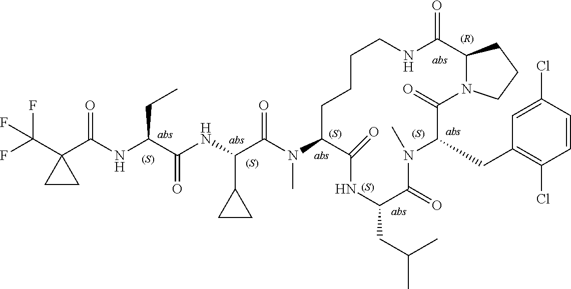 |
| 350 | 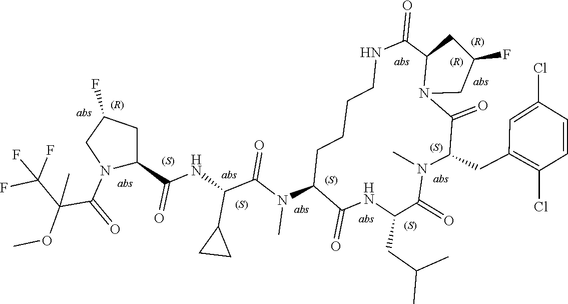 |
| 351 | 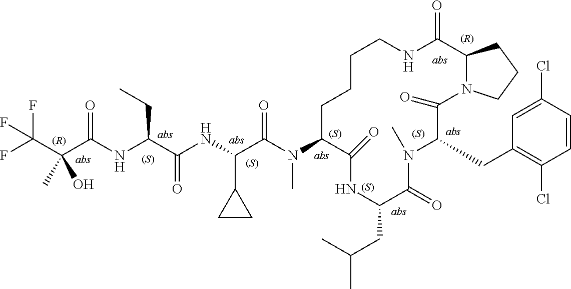 |
| 352 | 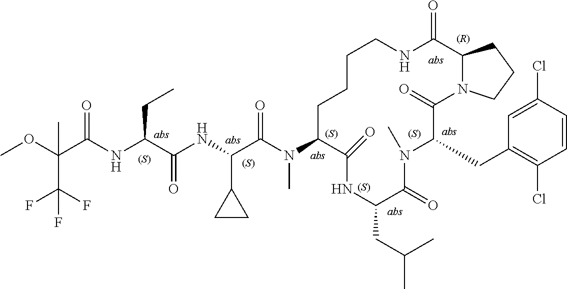 |
| 353 | 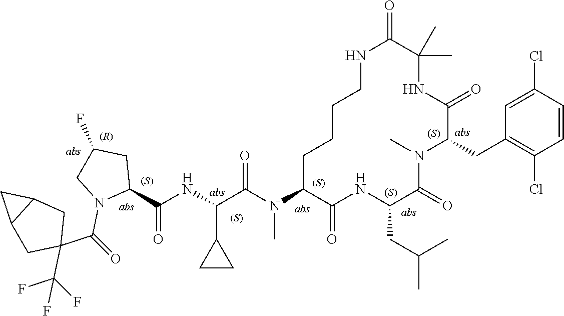 |
| 354 | 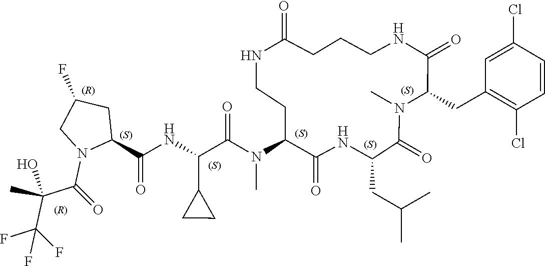 |
| 355 | 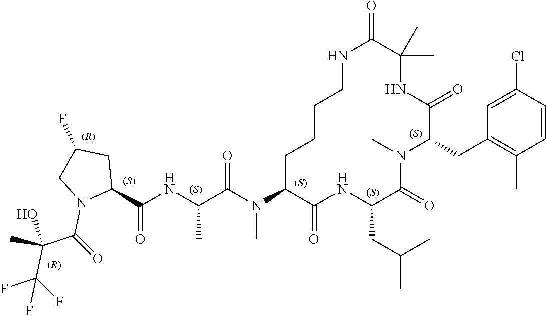 |
| 356 | 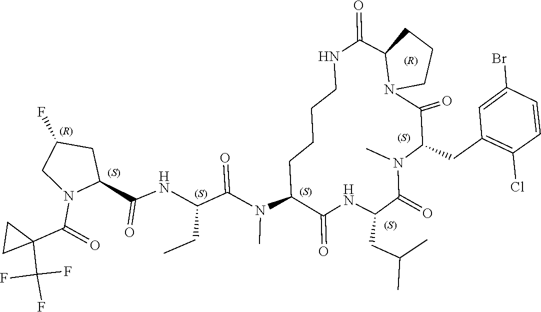 |
| 357 | 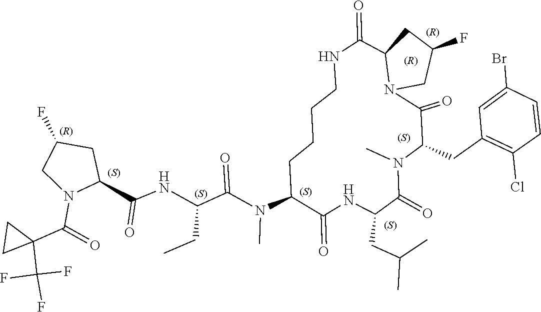 |
| 358 | 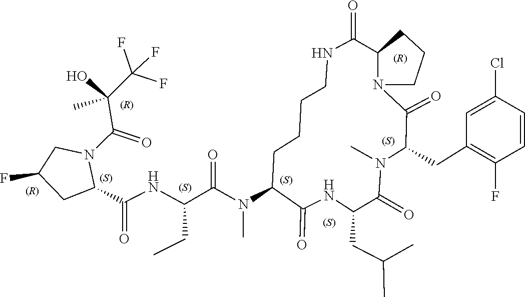 |
| 359 | 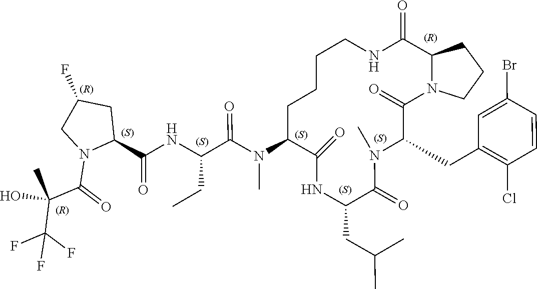 |
| 360 | 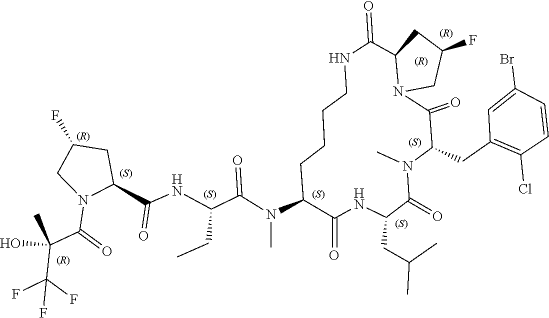 |
| 361 | 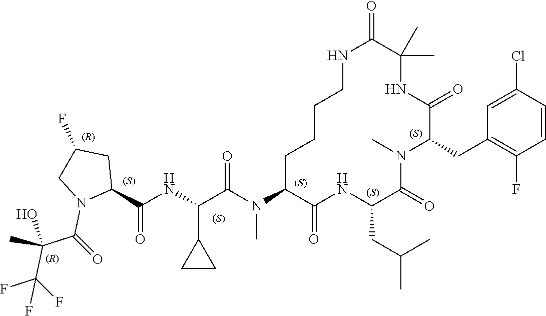 |
| 362 | 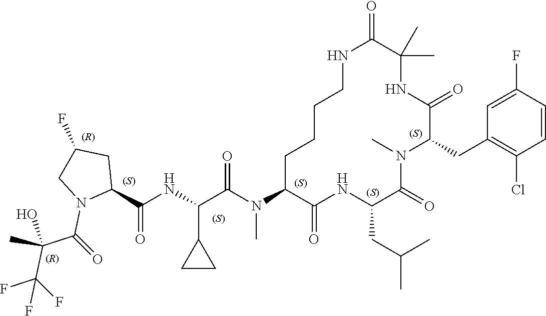 |
| 363 | 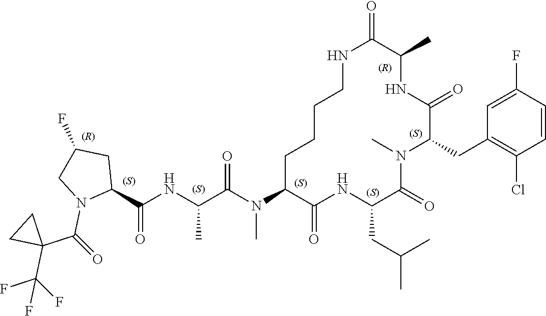 |
| 364 | 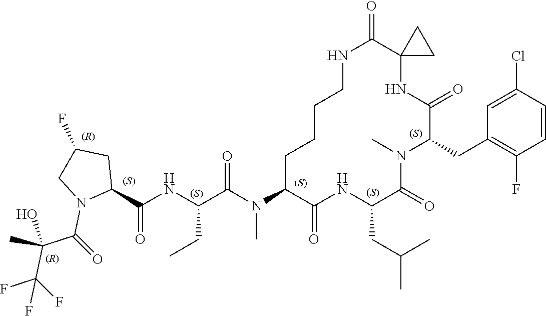 |
| 365 | 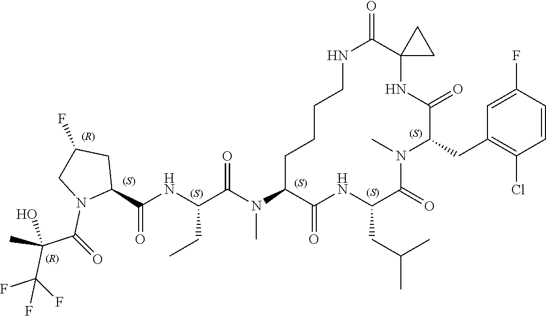 |
| 366 | 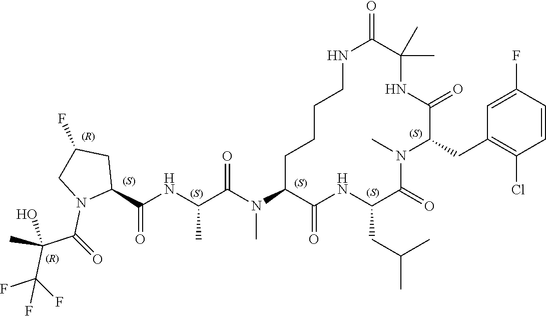 |
| 367 | 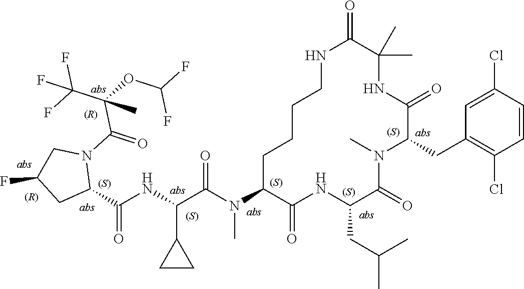 |
| 368 | 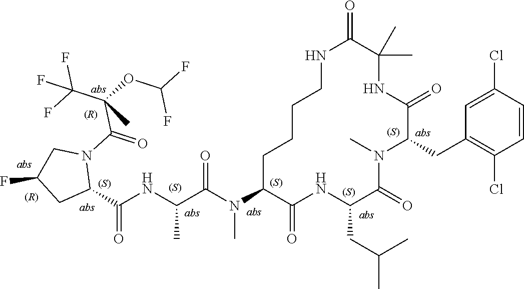 |
| 369 | 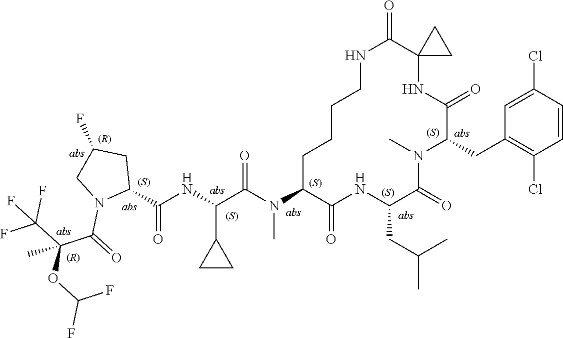 |
| 370 | 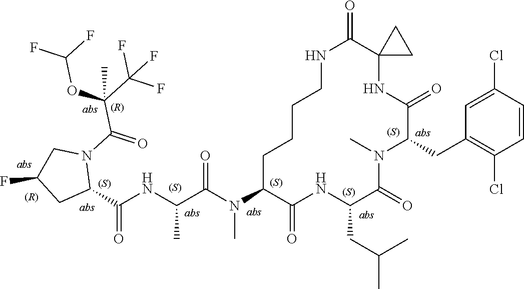 |
| 371 | 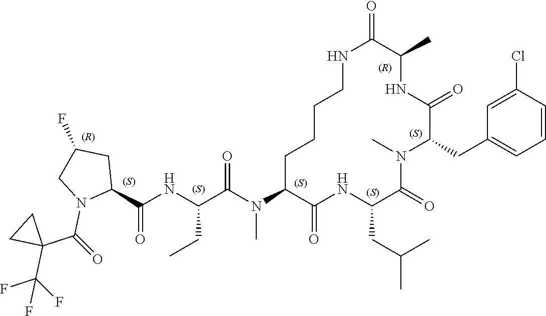 |
| 372 | 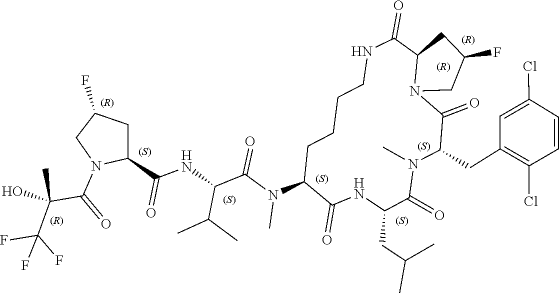 |
| 373 | 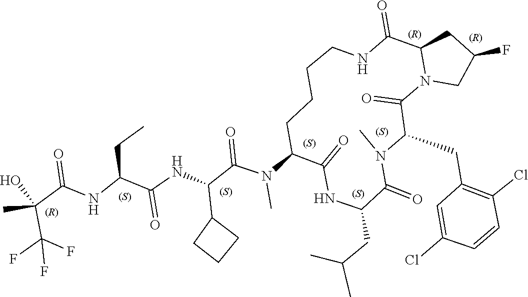 |
| 374 | 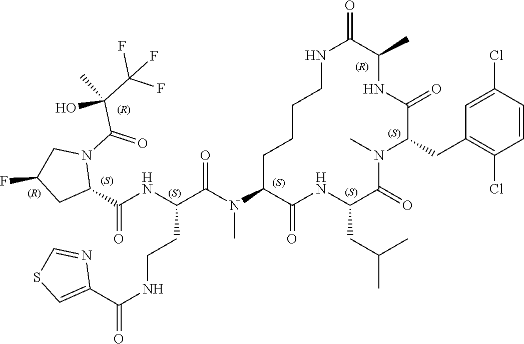 |
| 375 | 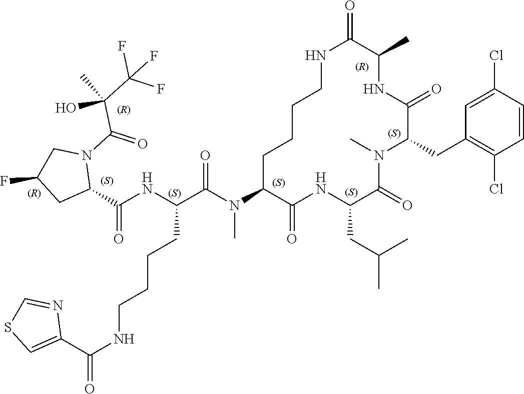 |
| 376 | 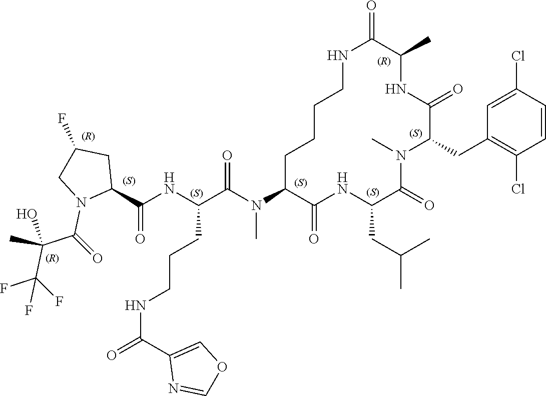 |
| 377 | 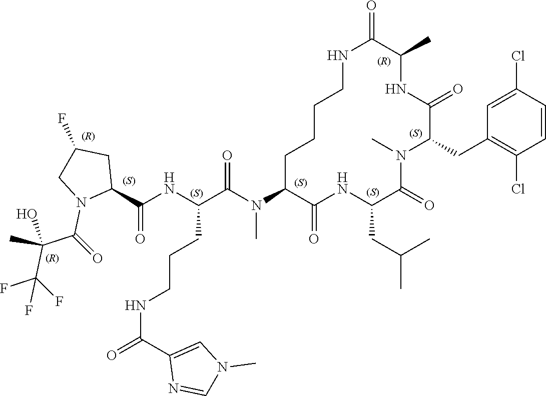 |
| 378 | 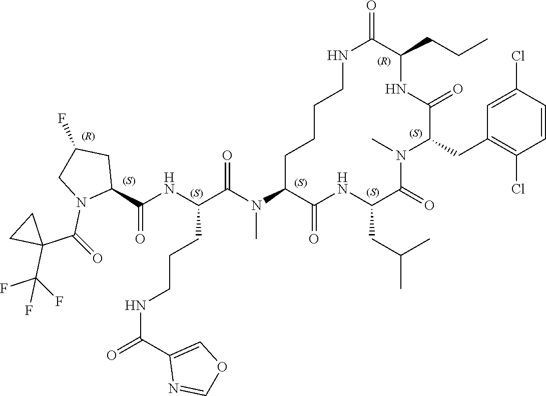 |
| 379 | 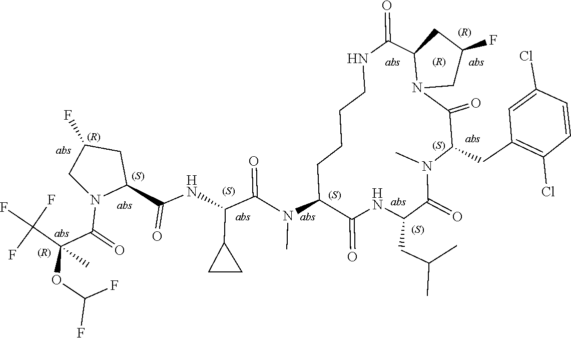 |
| 380 | 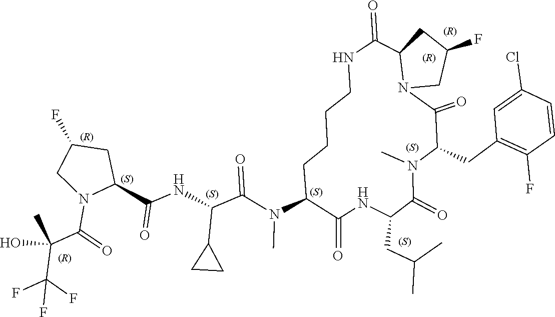 |
| 381 | 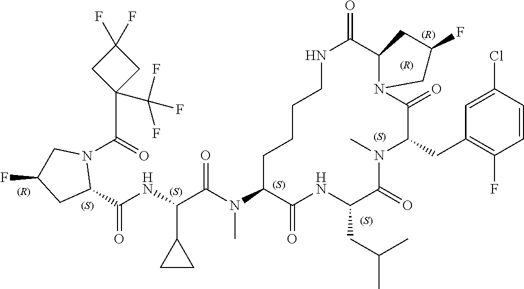 |
| 382 | 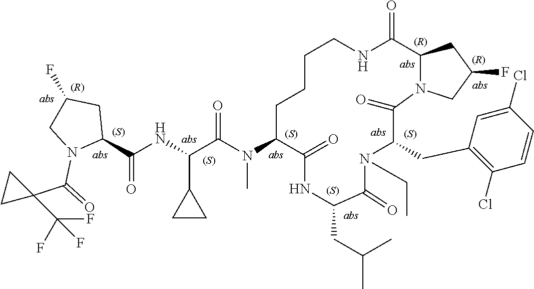 |
| 383 | 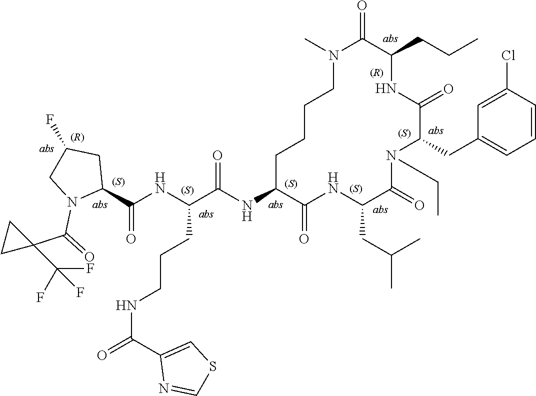 |
| 384 | 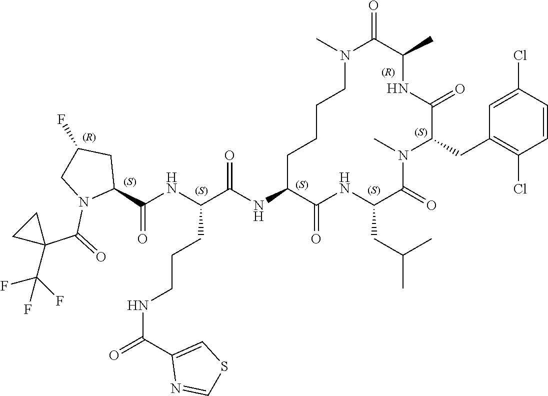 |
| 385 | 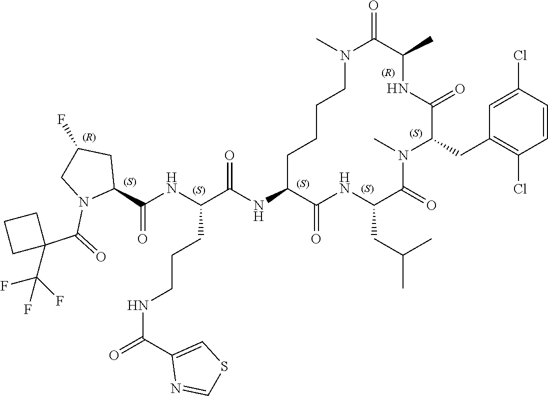 |
| 386 | 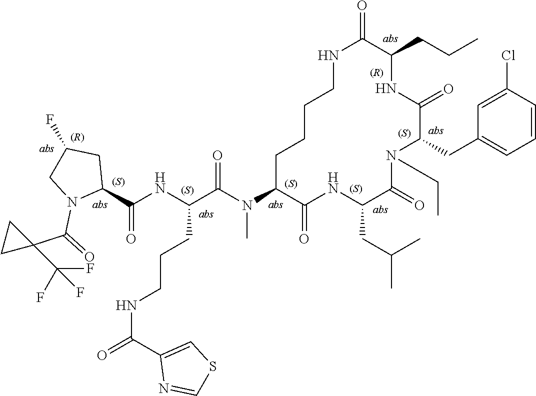 |
| 387 | 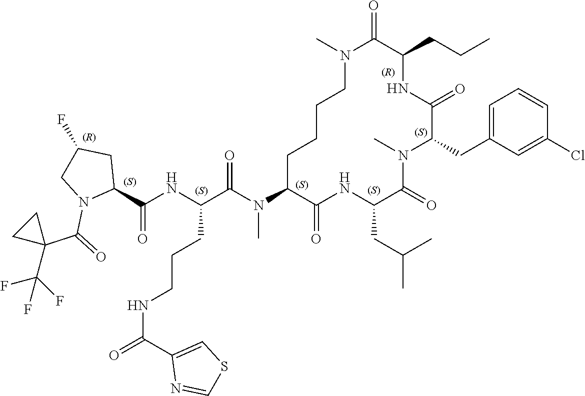 |
| 388 | 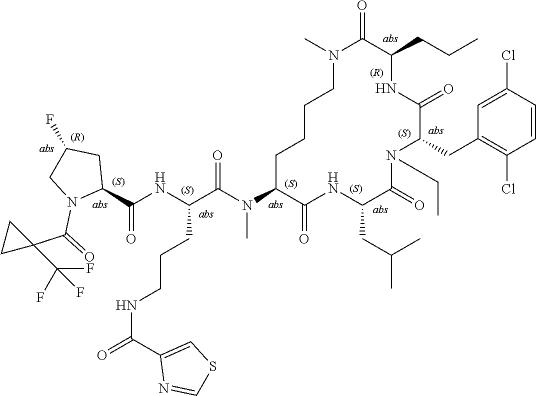 |
| 389 | 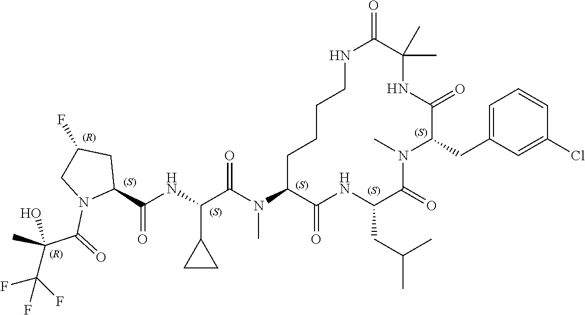 |
| 390 | 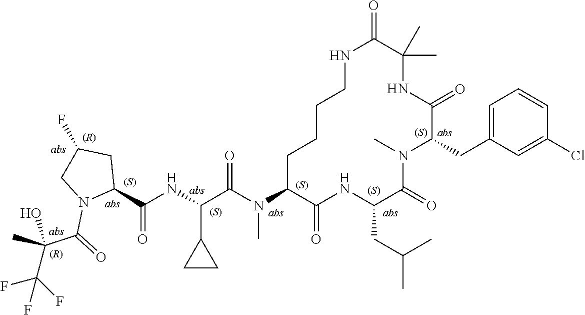 |
| 391 | 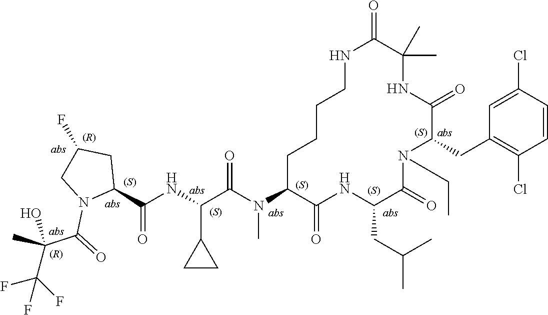 |
| 392 | 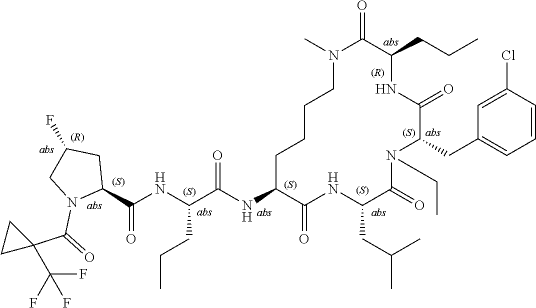 |
| 393 | 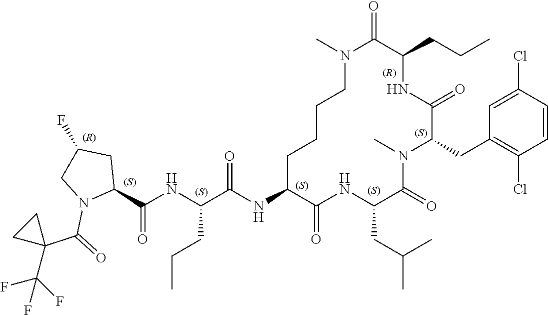 |
| 394 | 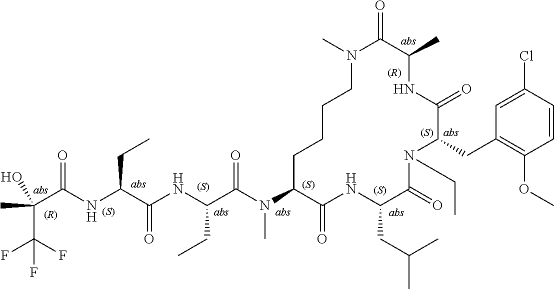 |
| 395 | 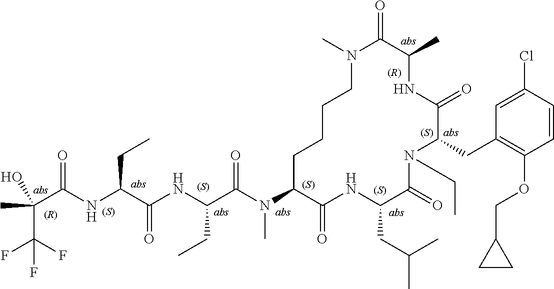 |
| 396 | 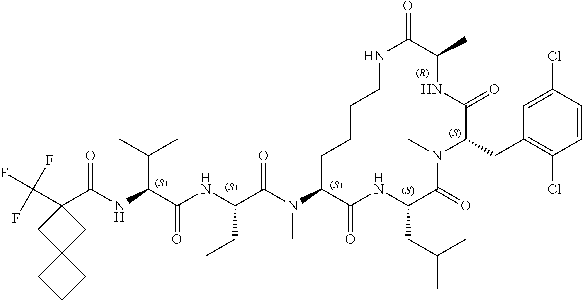 |
| 397 | 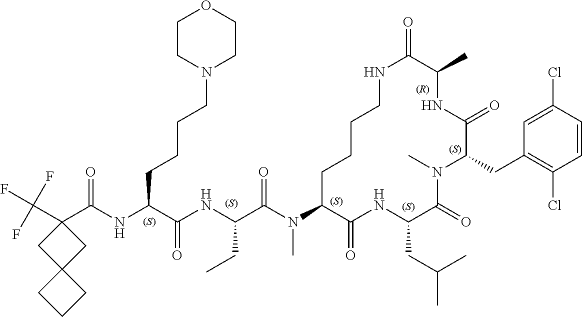 |
| 398 | 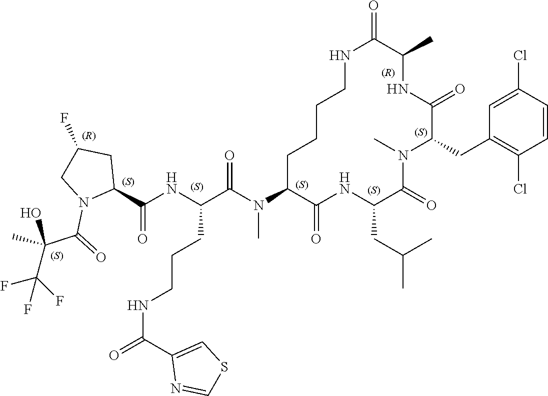 |
| 399 | 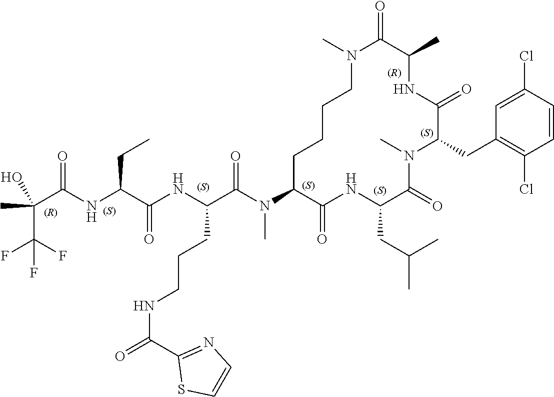 |
| 400 | 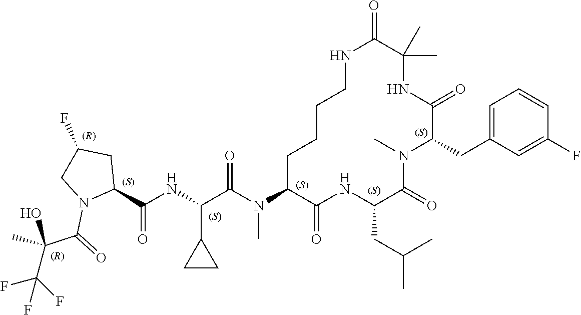 |
| 401 | 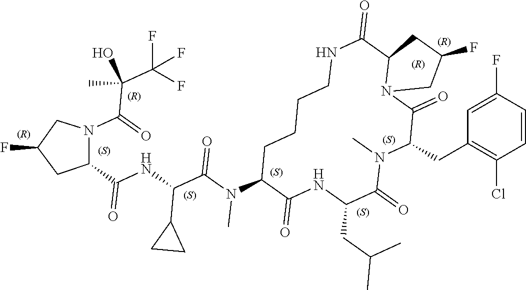 |
| 402 | 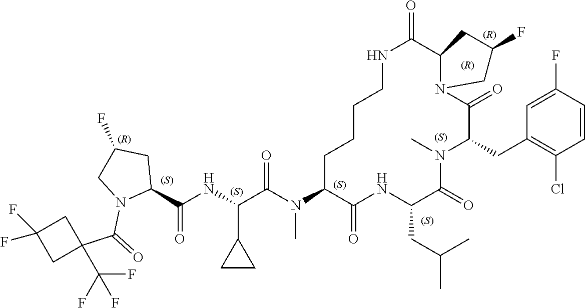 |
| 403 | 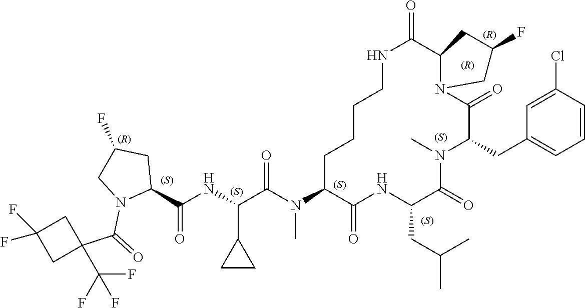 |
| 404 | 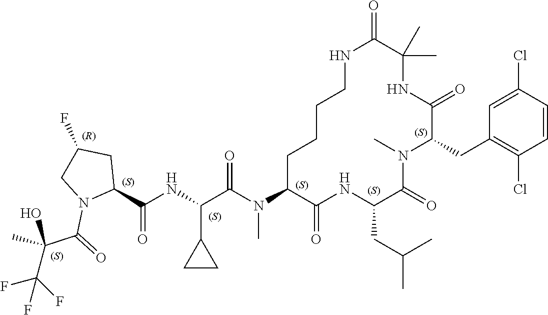 |
| 405 | 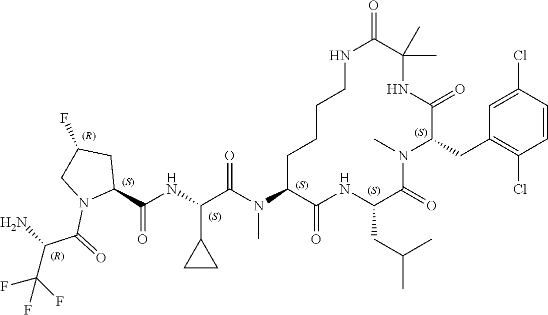 |
| 406 | 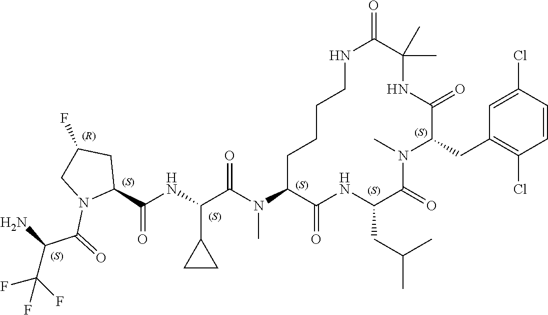 |
| 407 | 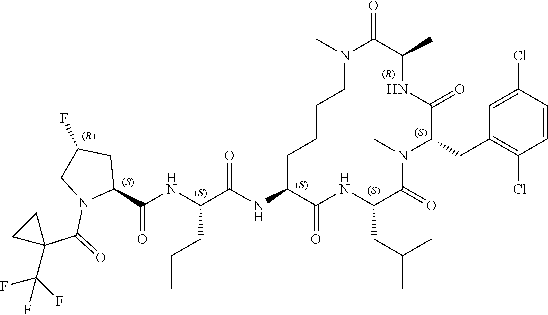 |
| 408 | 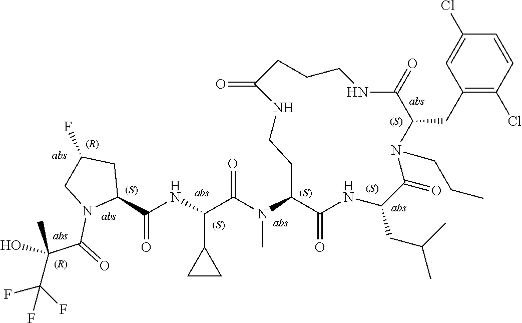 |
| 409 | 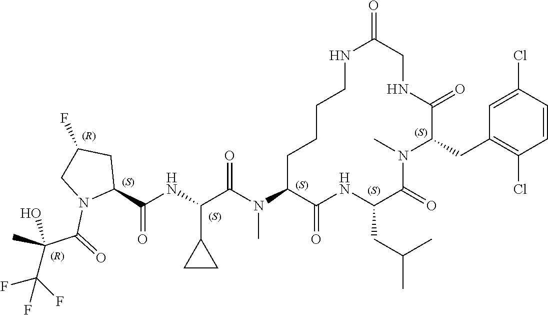 |
| 410 | 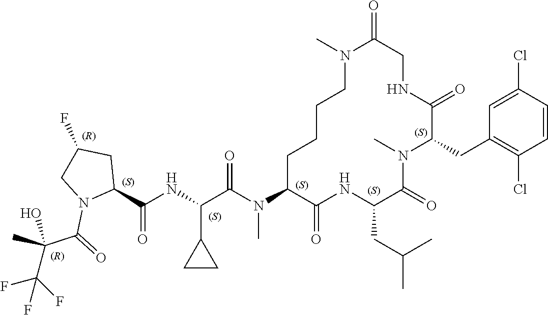 |
| 411 | 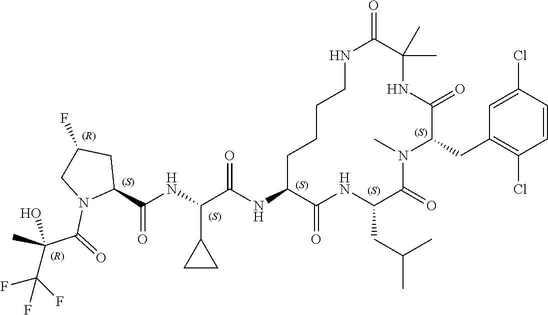 |
| 412 | 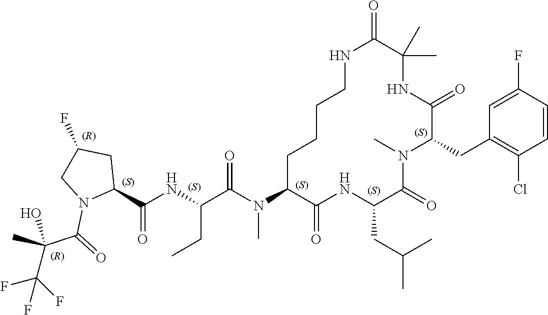 |
| 413 | 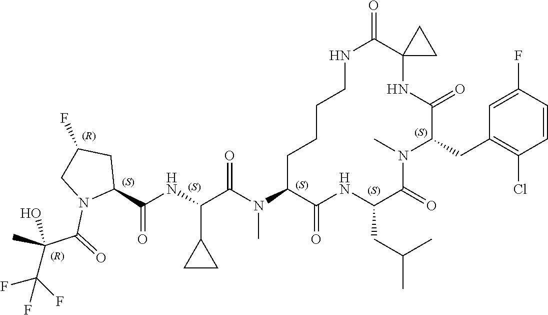 |
| 414 | 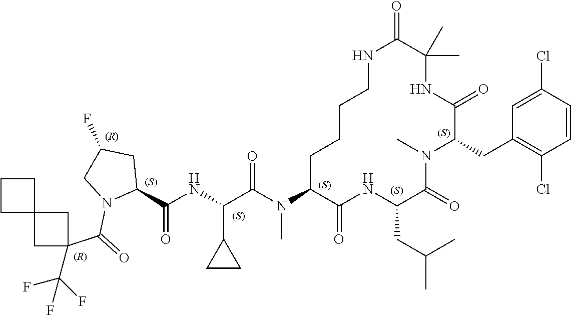 |
| 415 | 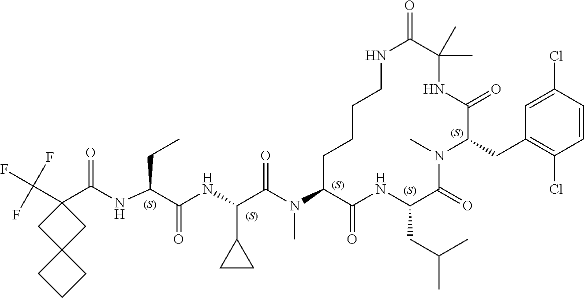 |
| 416 | 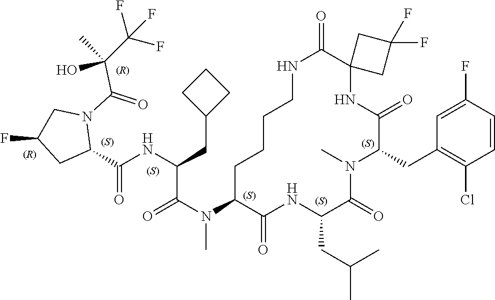 |
| 417 | 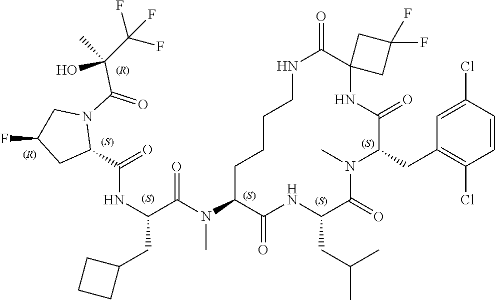 |
| 418 | 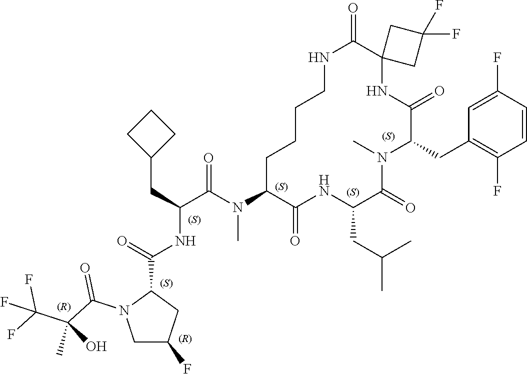 |
| 419 | 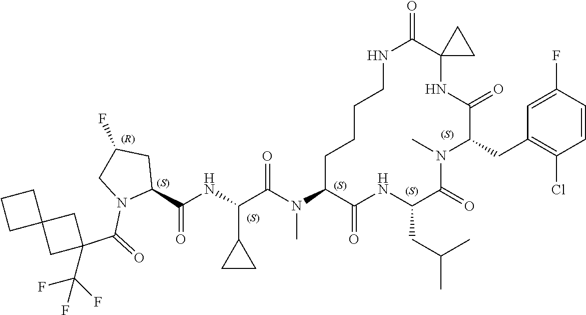 |
| 420 | 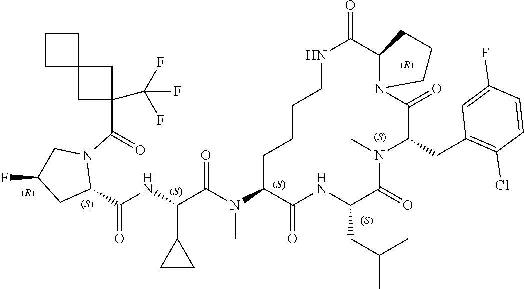 |
| 421 | 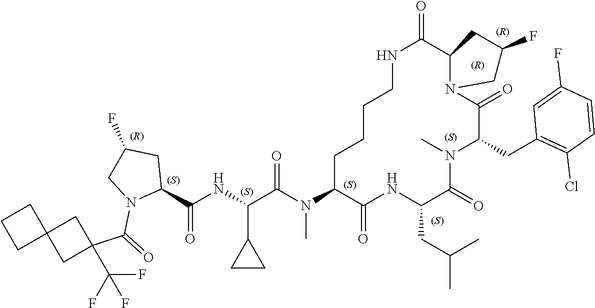 |
| 422 | 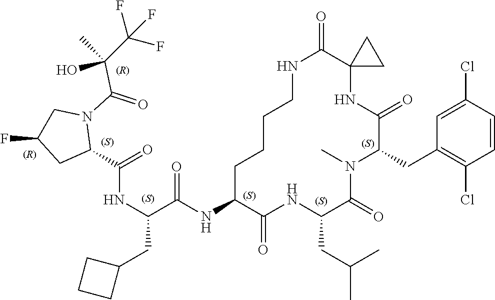 |
| 423 | 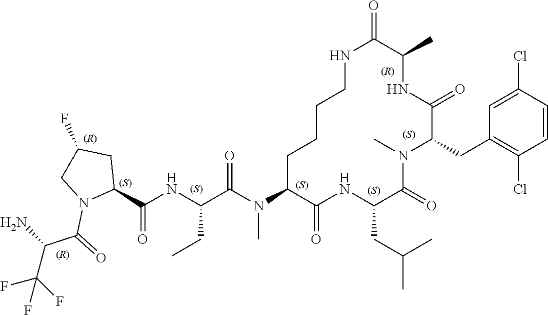 |
| 424 | 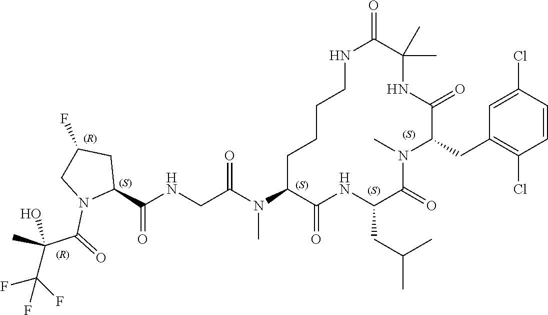 |
| 425 | 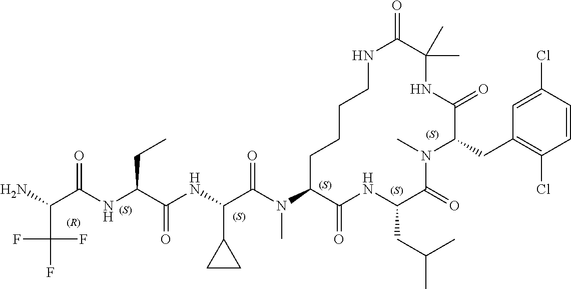 |
| 426 | 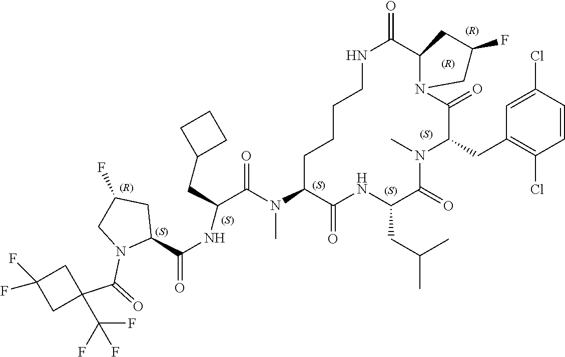 |
| 427 | 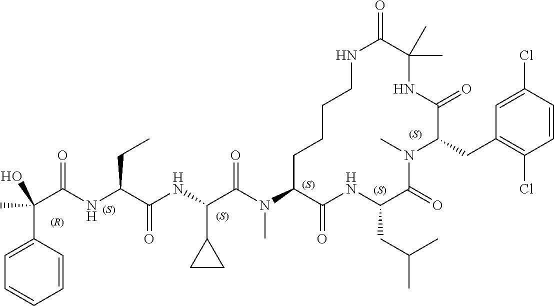 |
| 428 | 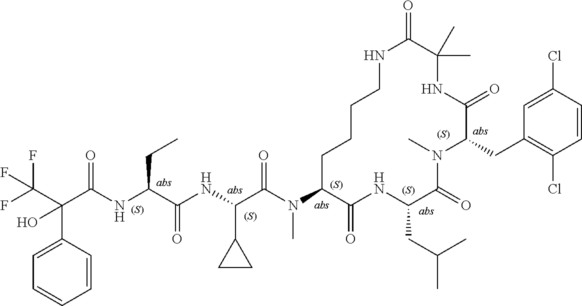 |
| 429 | 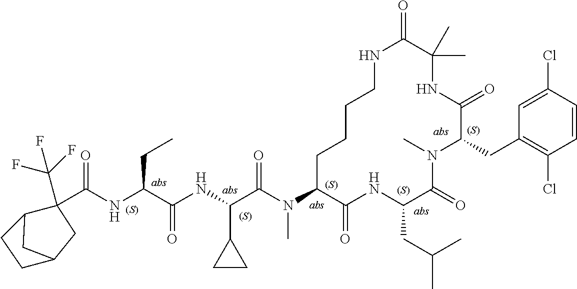 |
| 430 | 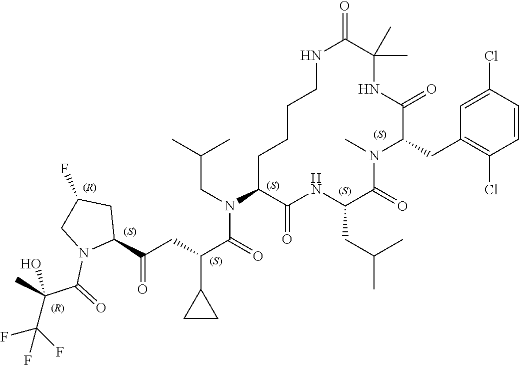 |
| 431 | 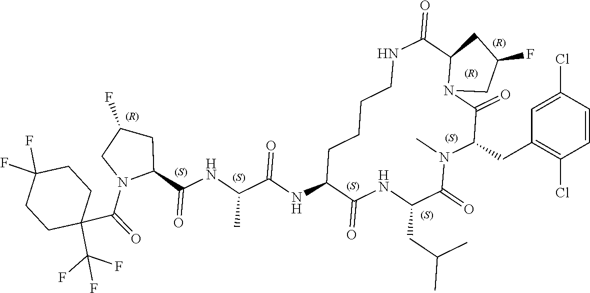 |
| 432 | 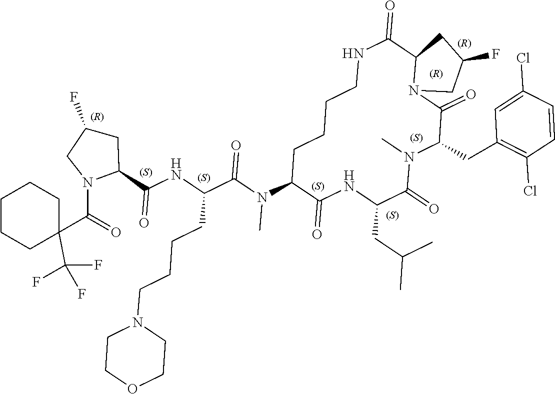 |
| 433 | 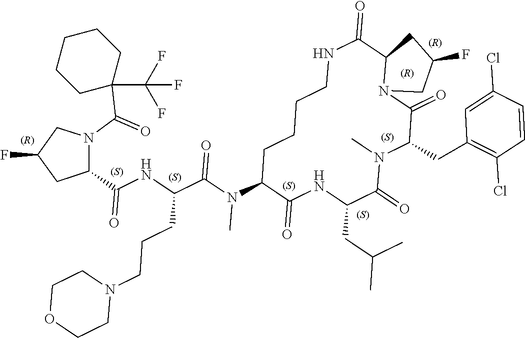 |
| 434 | 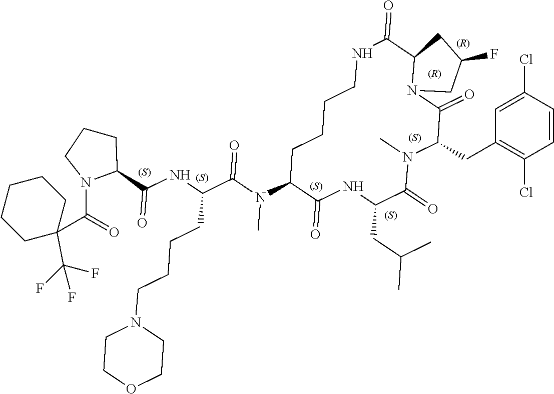 |
| 435 | 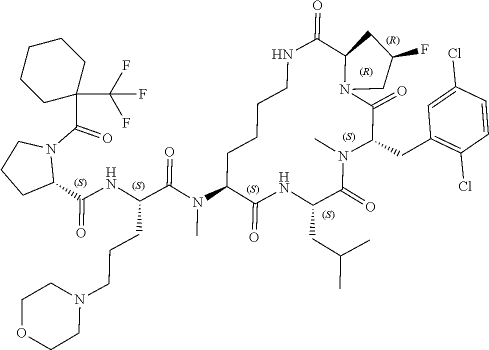 |
| 436 | 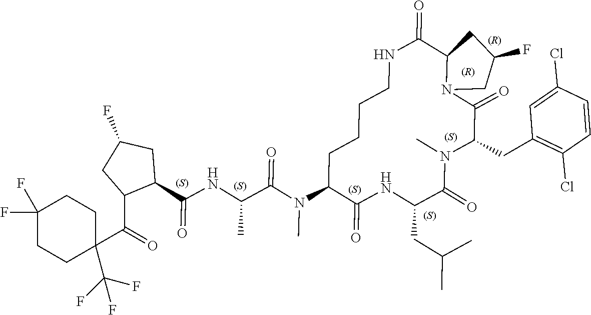 |
| 437 | 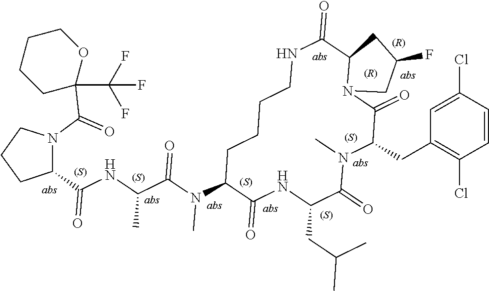 |
| 438 | 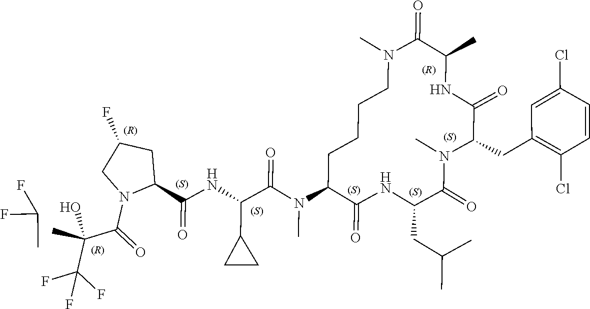 |
| 439 | 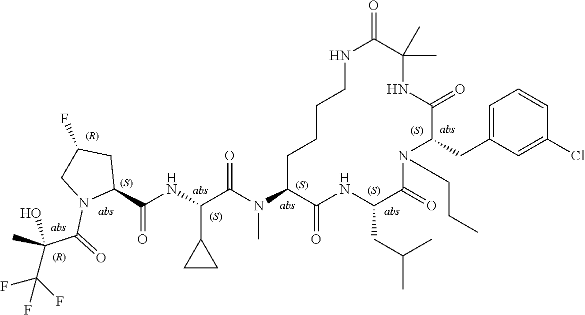 |
| 440 | 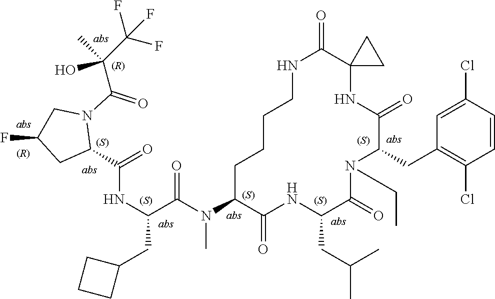 |
| 441 | 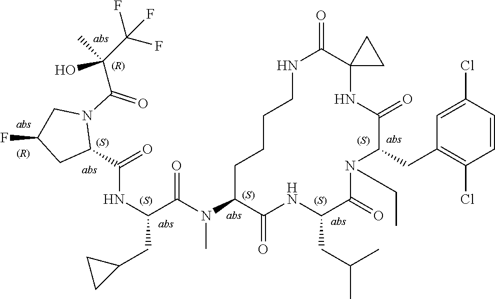 |
| 442 | 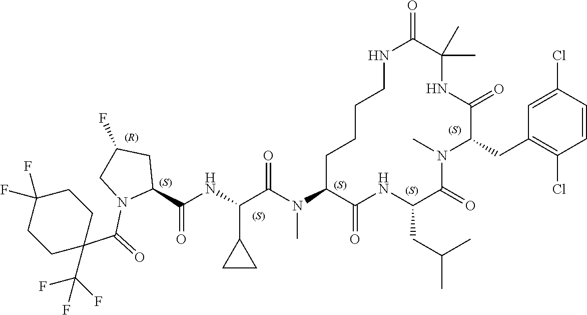 |
| 443 | 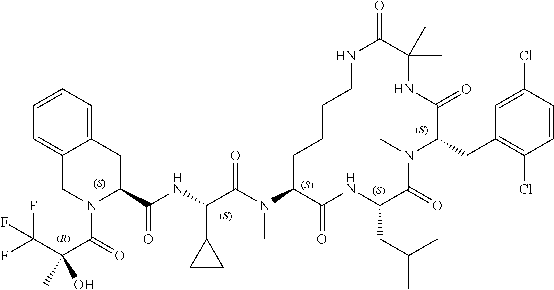 |
| 444 | 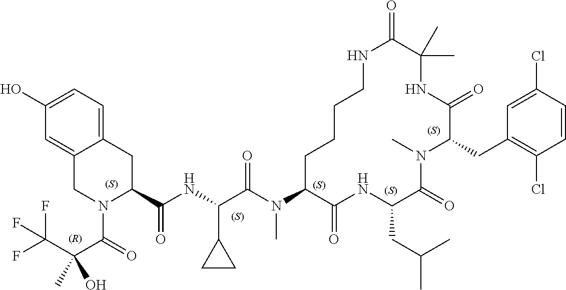 |
| 445 | 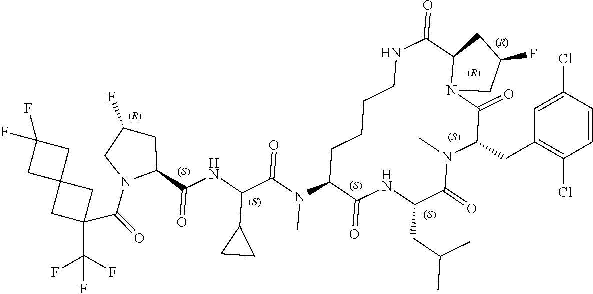 |
| 446 | 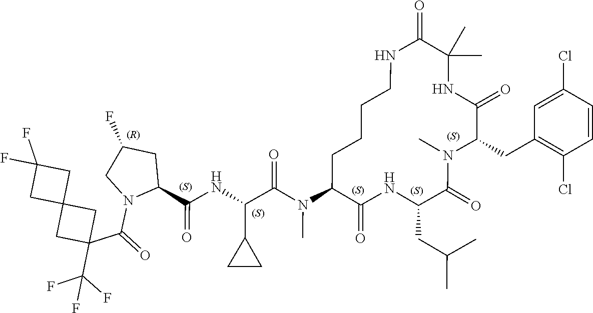 |
| 447 | 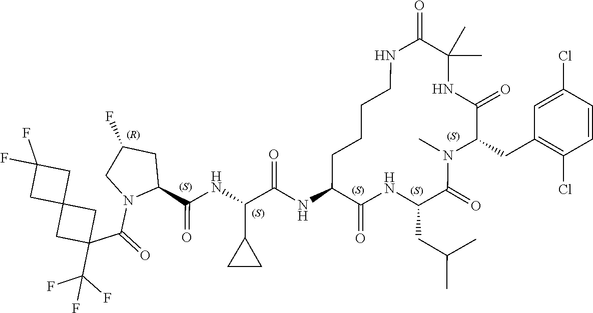 |
| 448 | 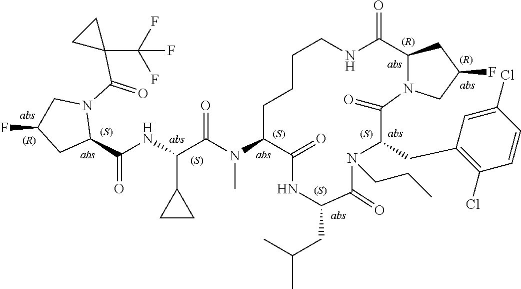 |
| 449 | 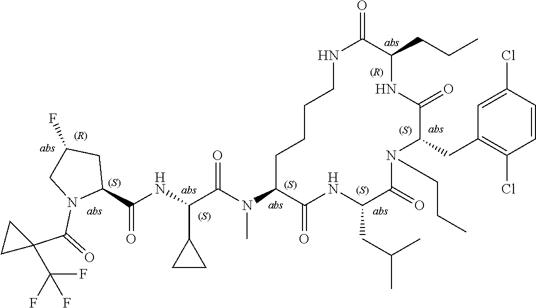 |
| 450 | 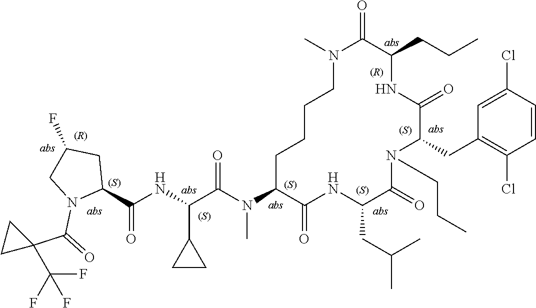 |
| 451 | 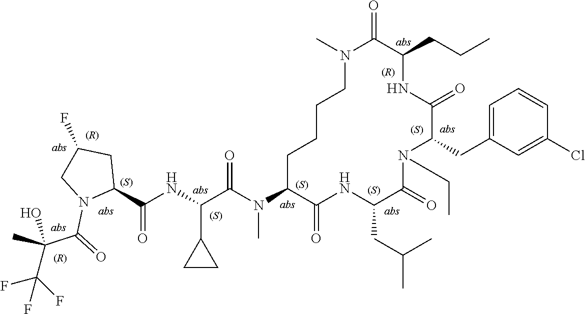 |
| 452 | 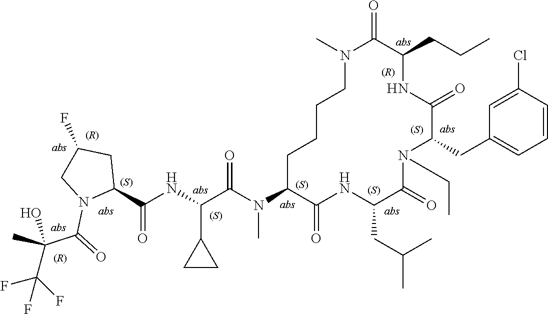 |
| 453 | 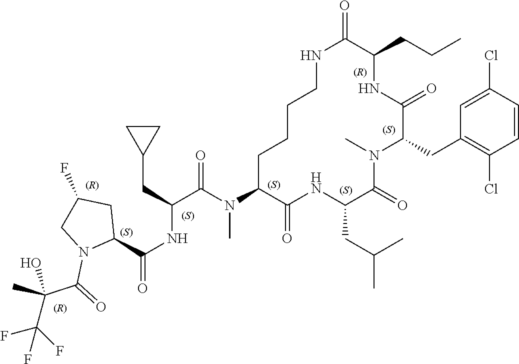 |
| 454 | 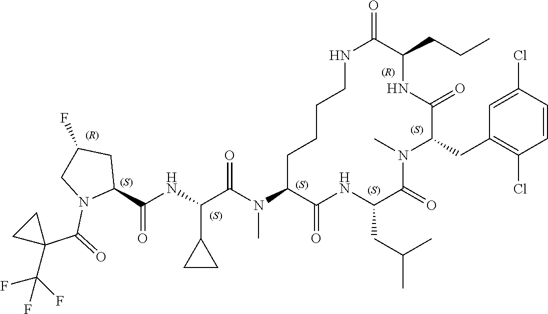 |
| 455 | 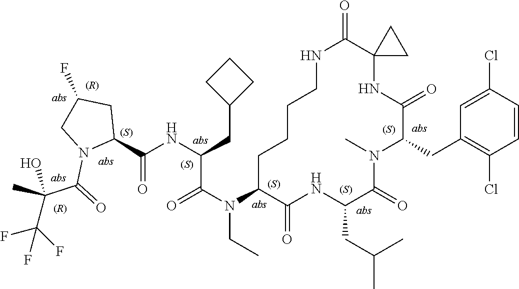 |
| 456 | 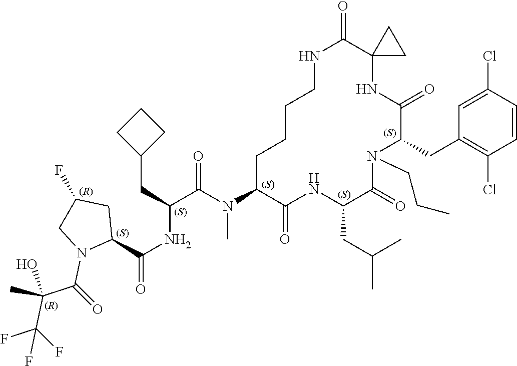 |
| 457 | 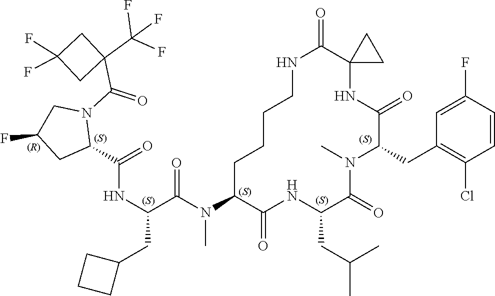 |
| 458 | 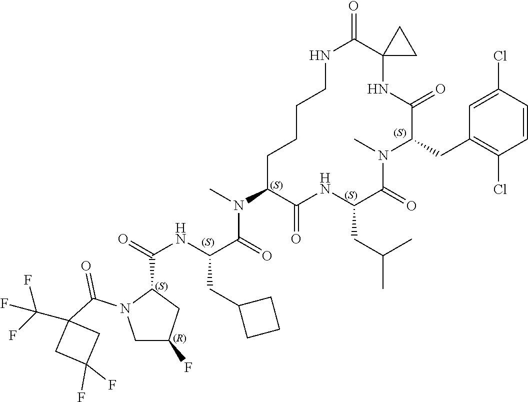 |
| 459 | 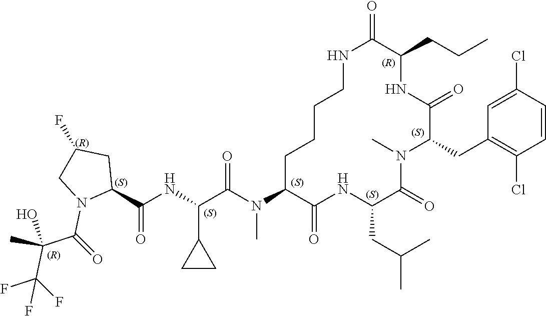 |
| 460 | 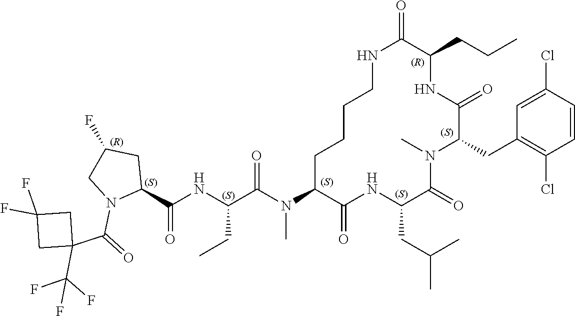 |
| 461 | 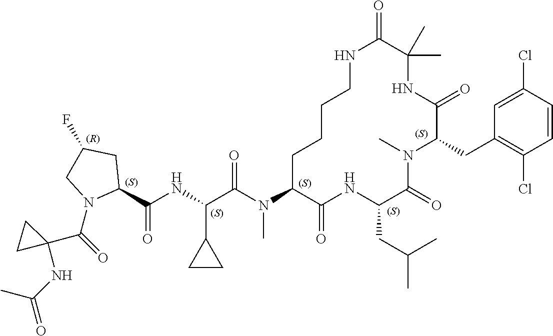 |
| 462 | 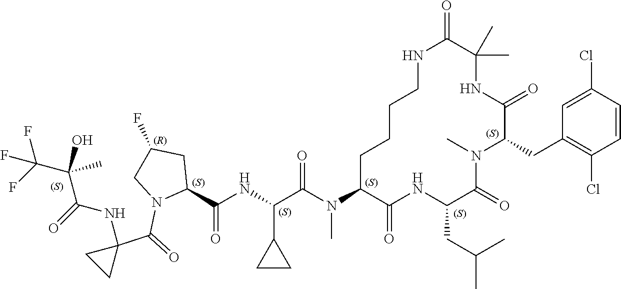 |
| 463 | 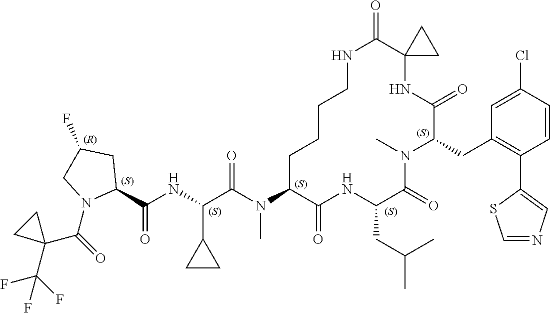 |
| 464 | 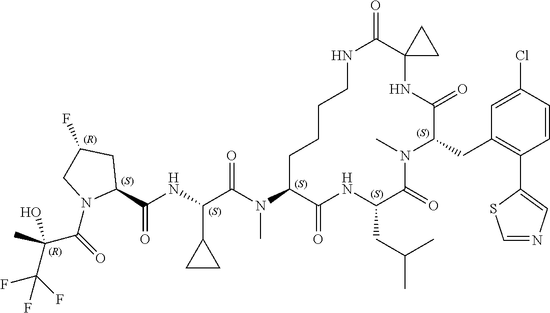 |
| 465 | 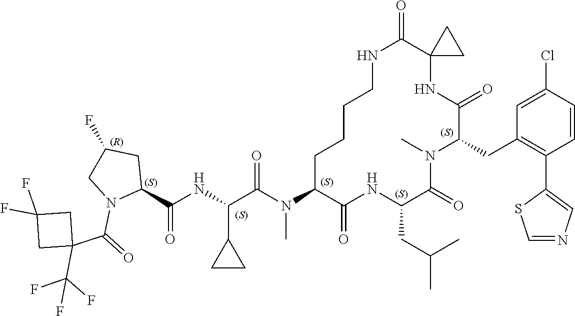 |
| 466 | 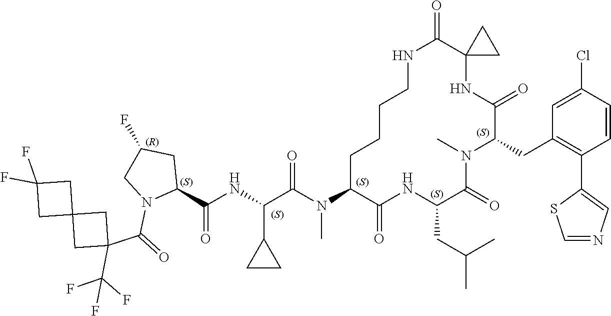 |
| 467 | 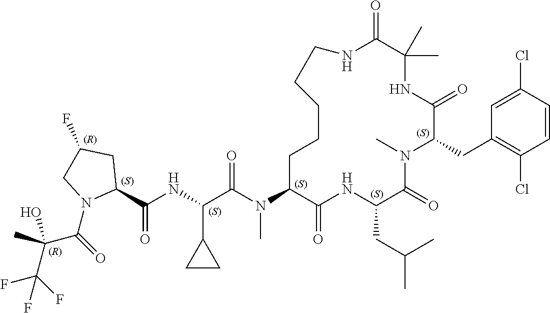 |
| 468 | 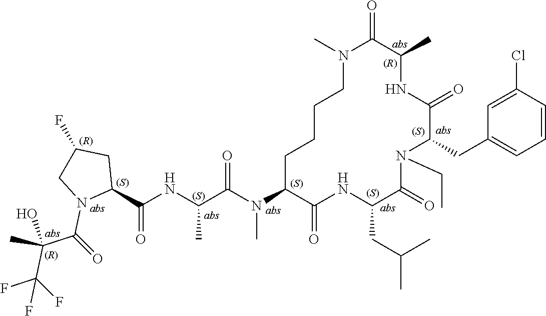 |
| 469 | 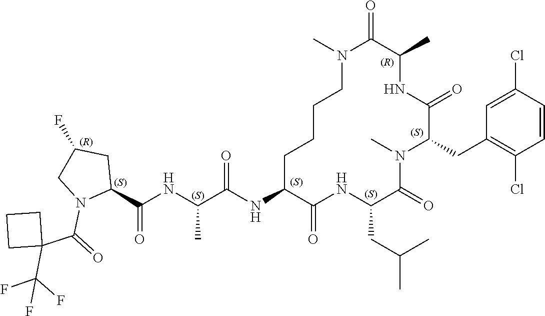 |
| 470 | 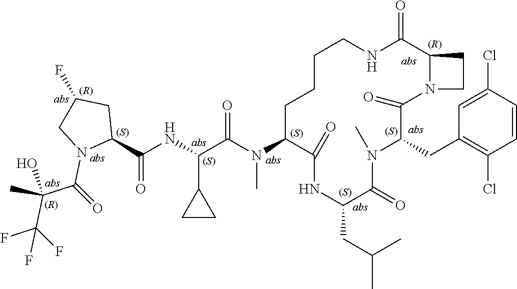 |
| 471 | 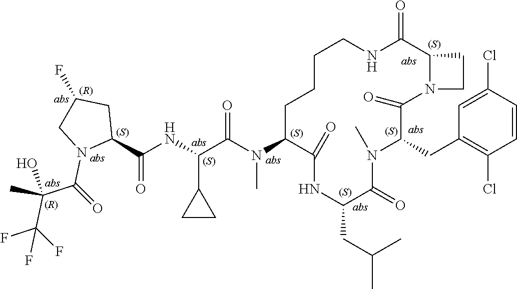 |
| 472 | 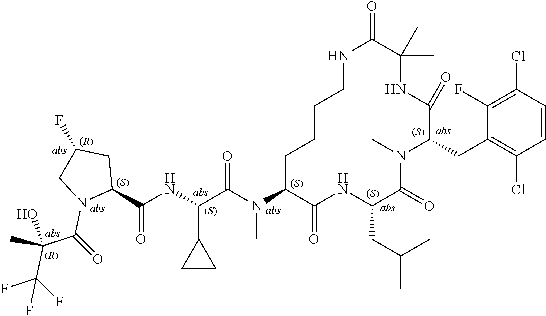 |
| 473 | 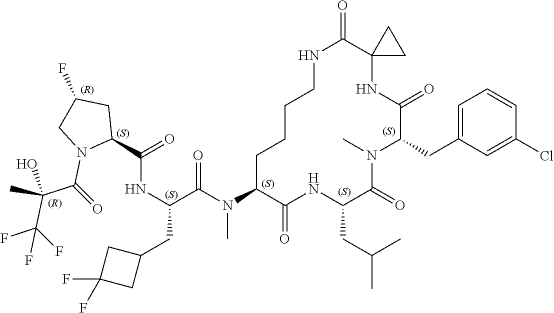 |
| 474 | 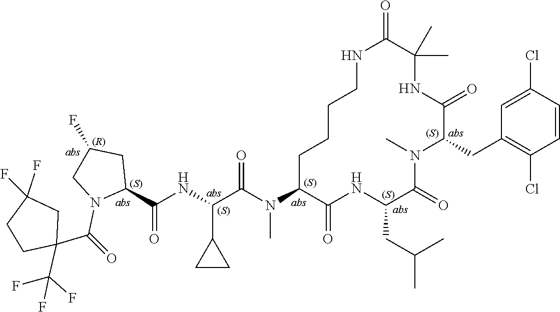 |
| 475 | 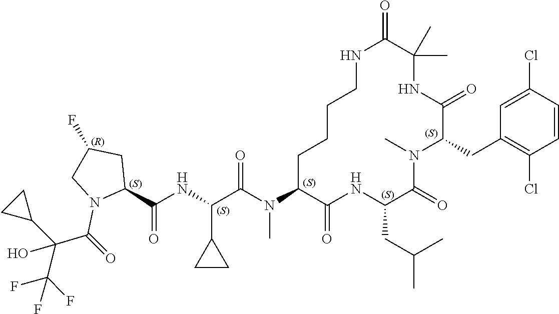 |
| 476 | 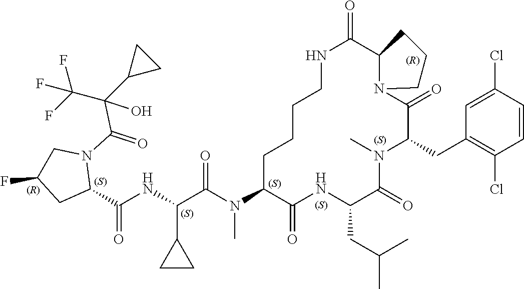 |
| 477 | 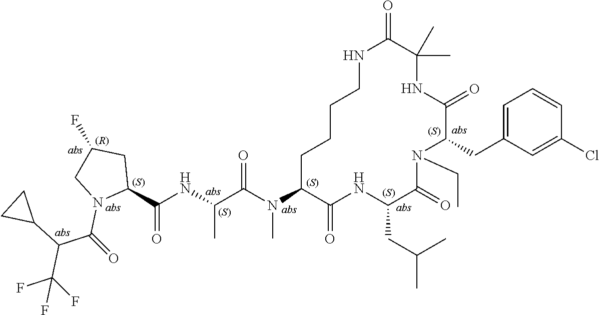 |
| 478 | 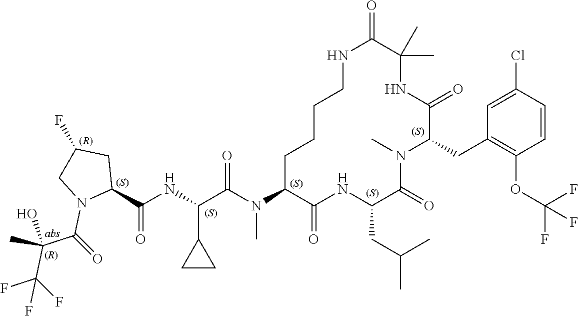 |
| 479 | 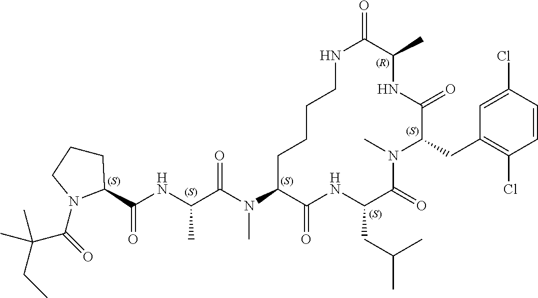 |
| 480 | 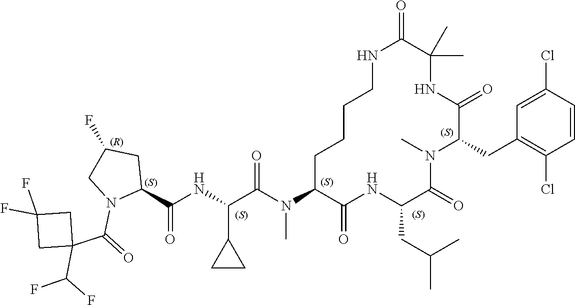 |
| 481 | 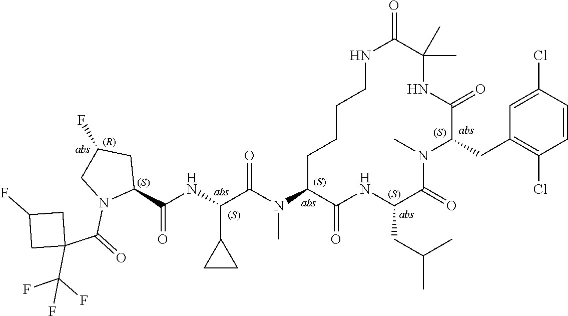 |
| 482 | 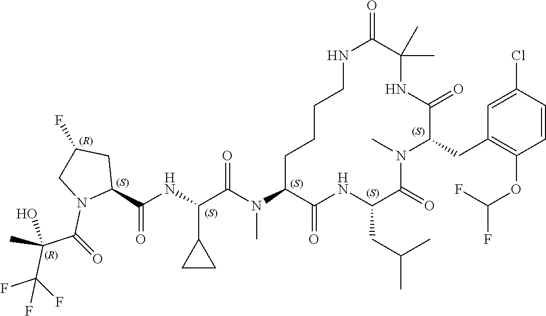 |
| 483 | 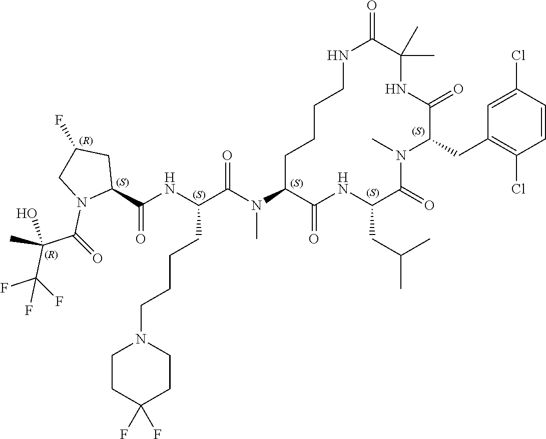 |
| 484 | 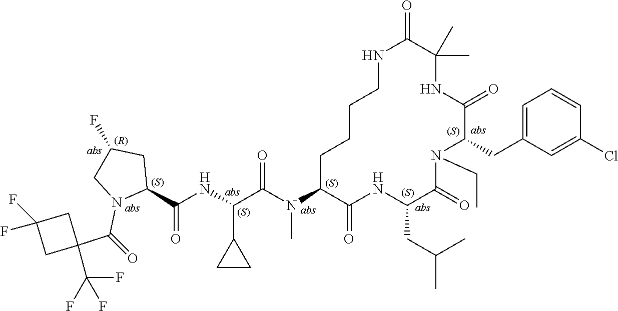 |
| 485 | 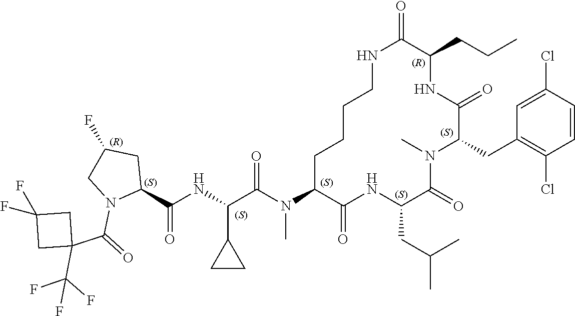 |
| 486 | 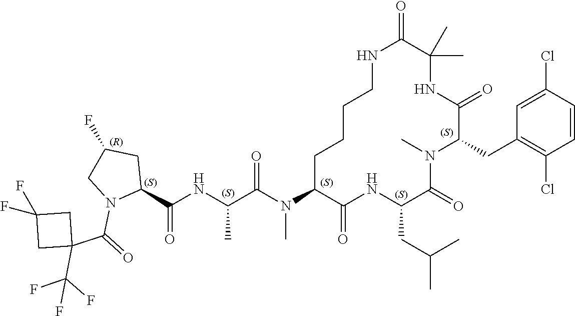 |
| 487 | 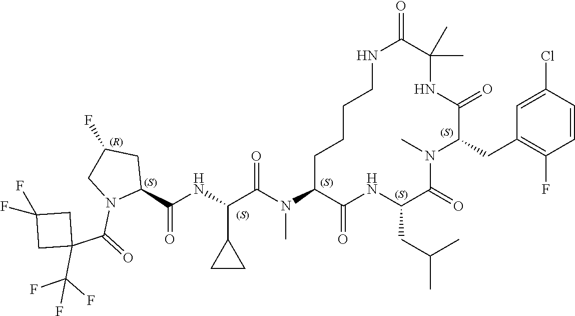 |
| 488 | 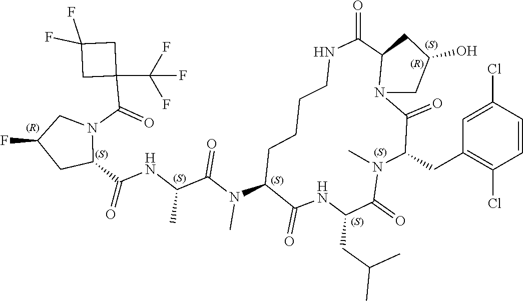 |
| 489 | 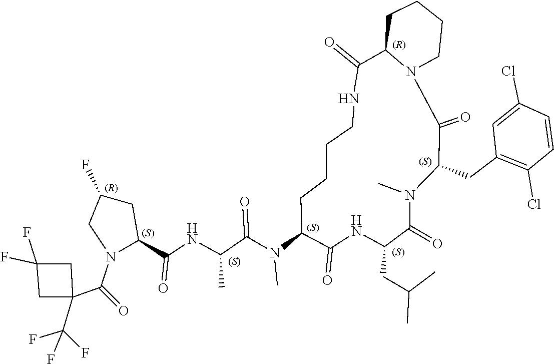 |
| 490 | 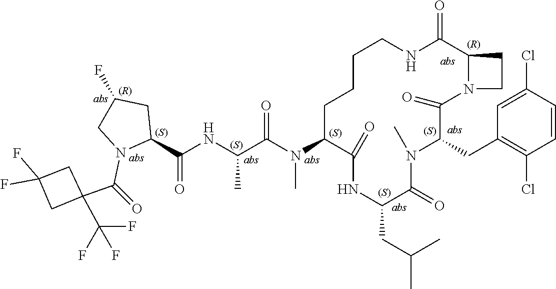 |
| 491 | 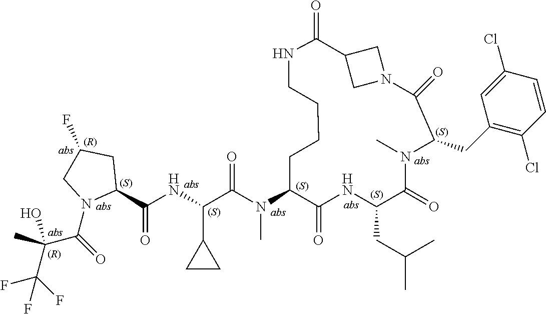 |
| 492 | 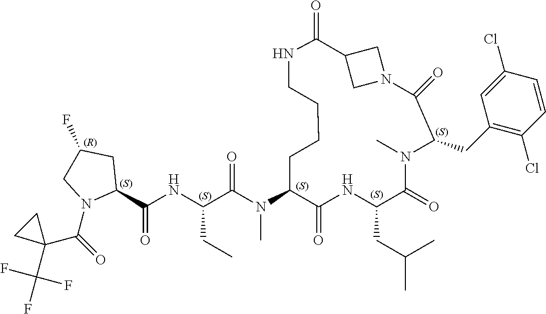 |
| 493 | 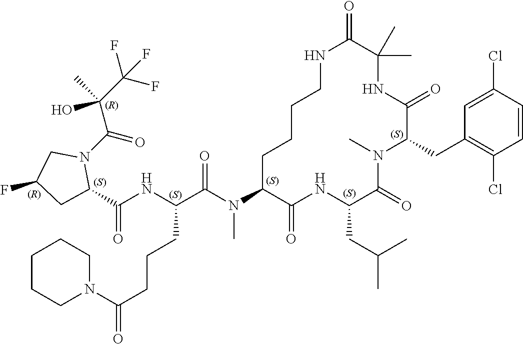 |
| 494 | 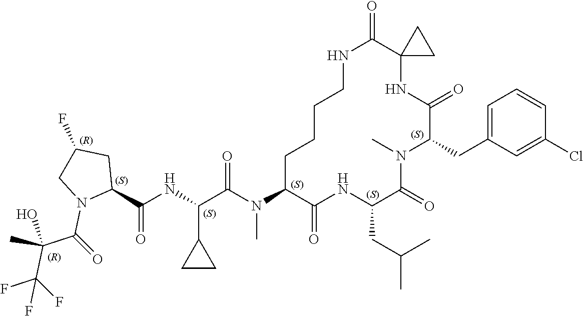 |
| 495 | 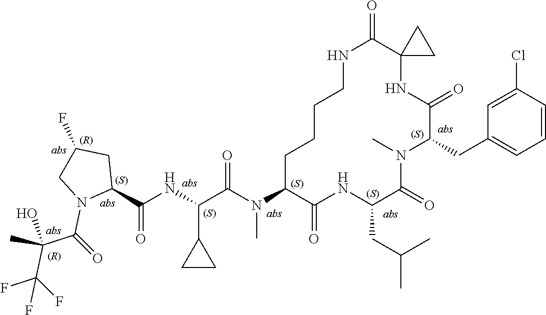 |
| 496 | 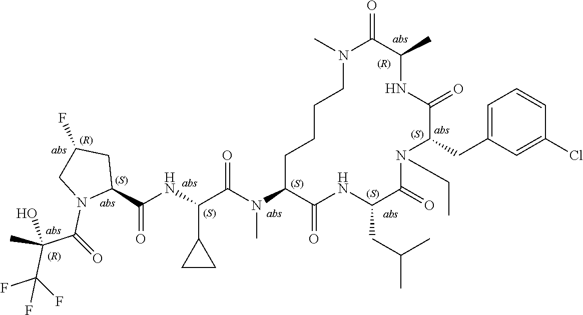 |
| 497 | 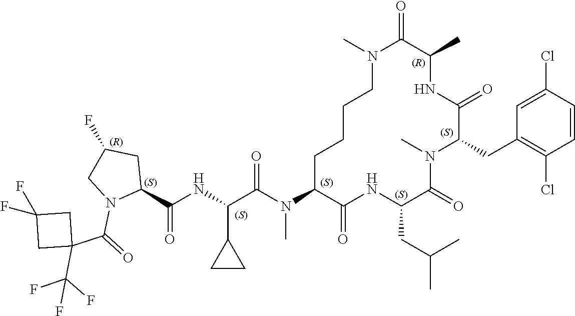 |
| 498 | 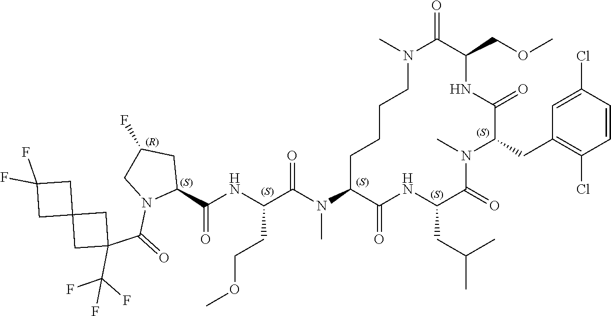 |
| 499 | 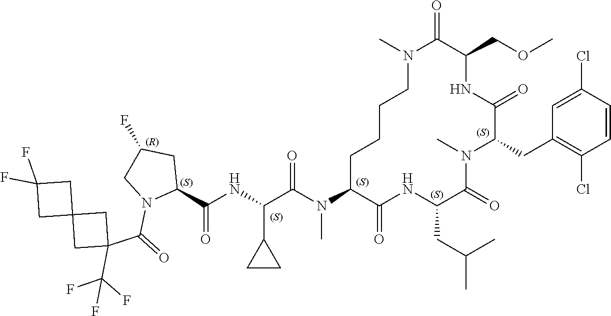 |
| 500 | 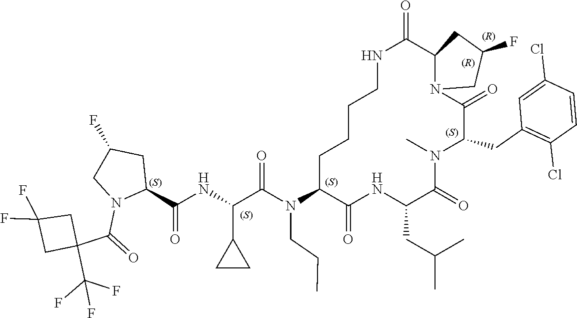 |
| 501 | 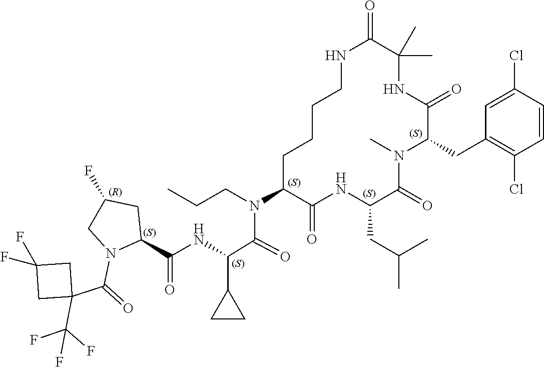 |
| 502 | 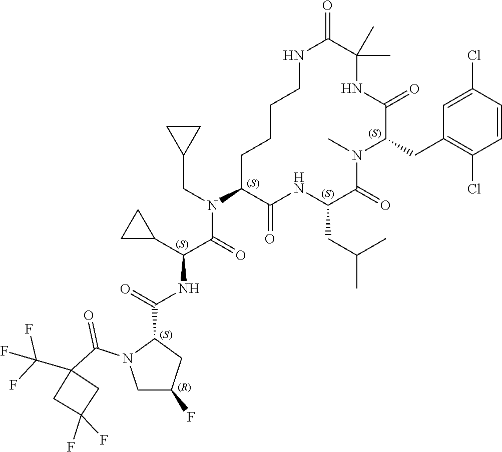 |
| 503 | 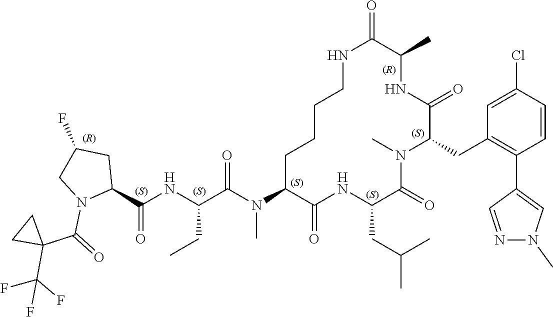 |
| 504 | 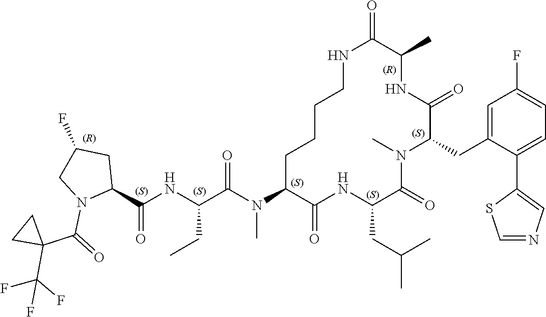 |
| 505 | 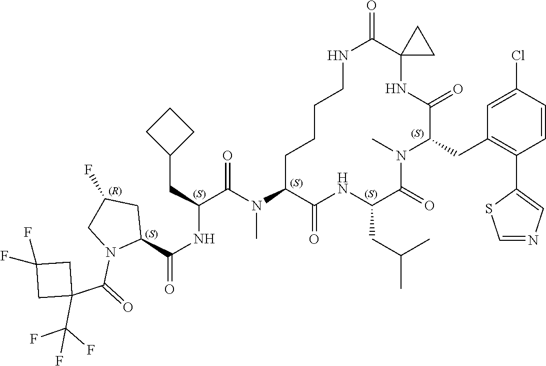 |
| 506 | 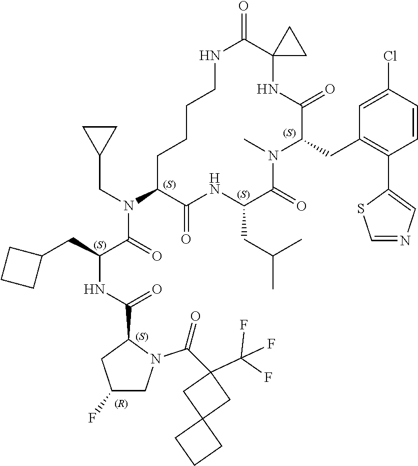 |
| 507 | 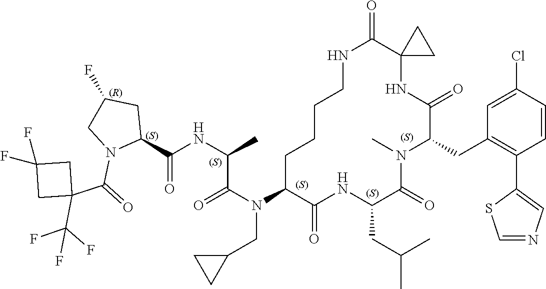 |
| 508 | 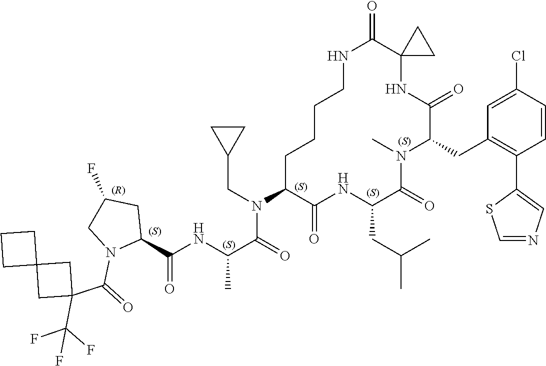 |
| 509 | 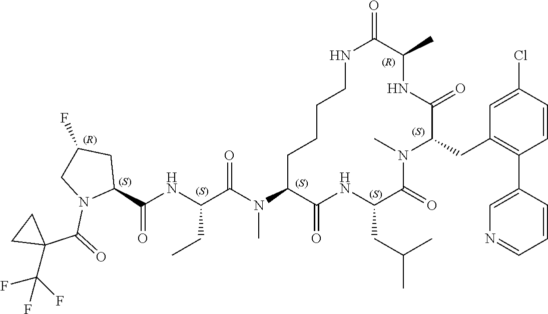 |
| 510 | 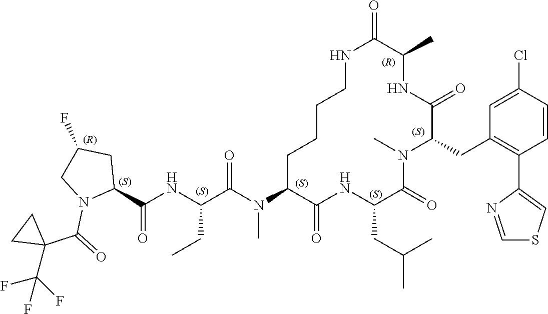 |
| 511 | 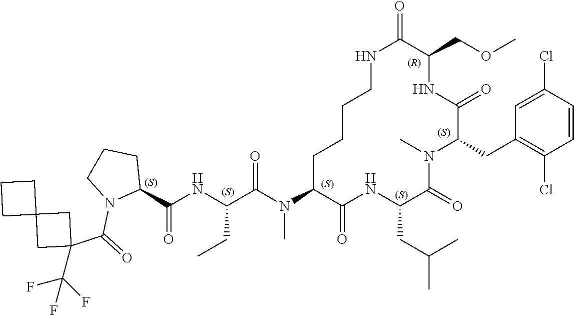 |
| 512 | 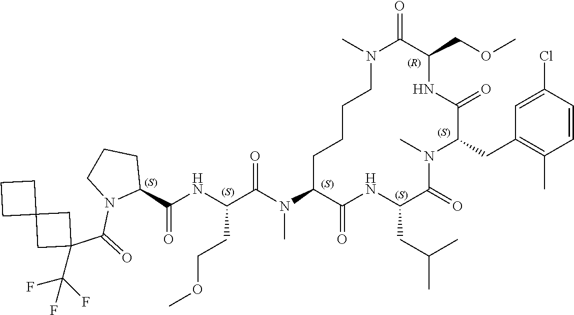 |
| 513 | 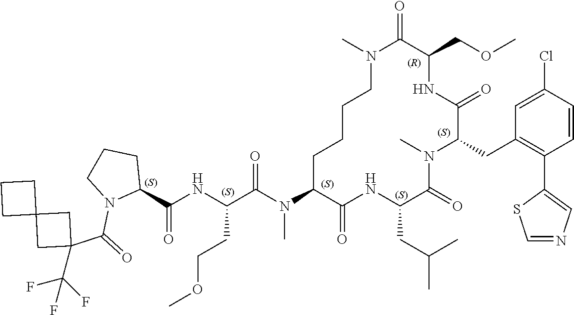 |
| 514 | 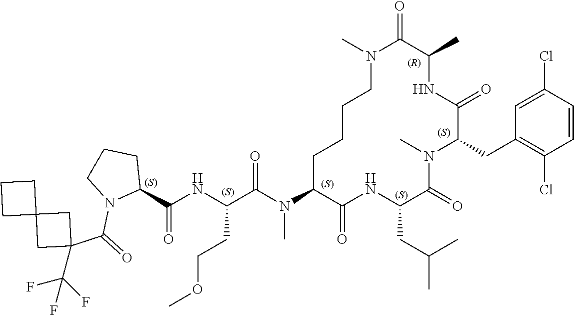 |
| 515 | 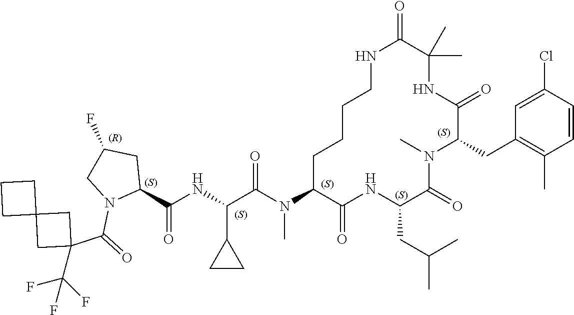 |
| 516 | 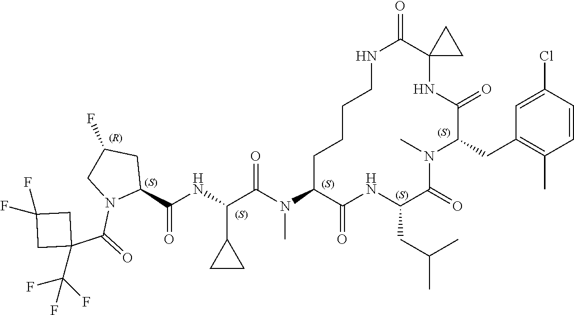 |
| 517 | 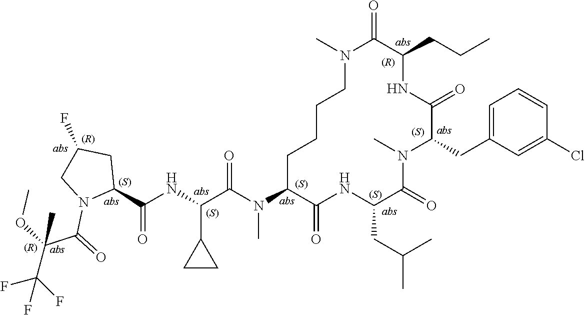 |
| 518 | 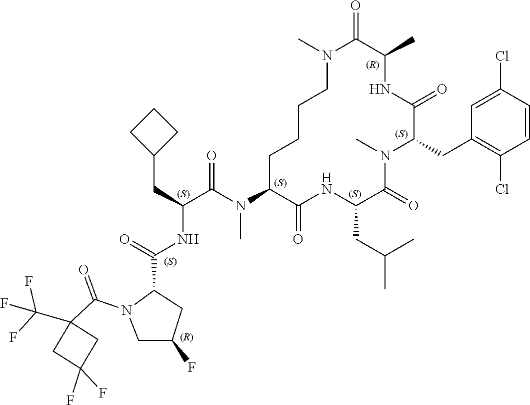 |
| 519 | 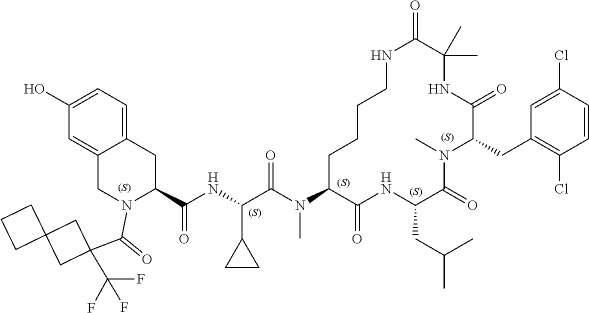 |
| 520 | 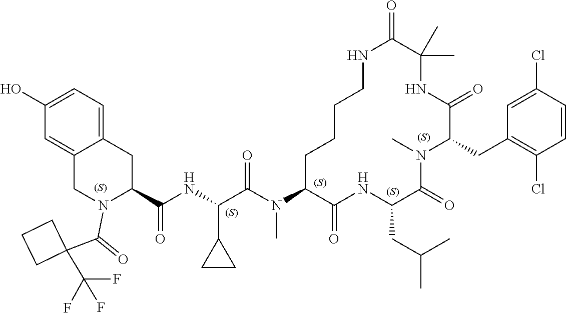 |
| 521 | 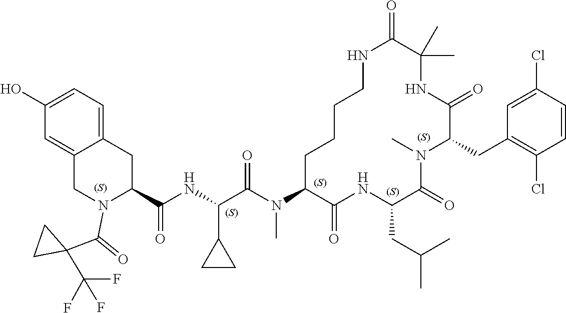 |
| 522 | 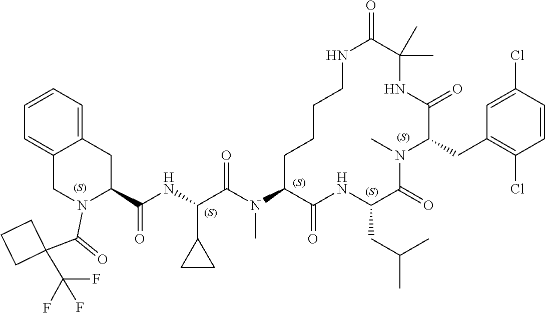 |
| 523 | 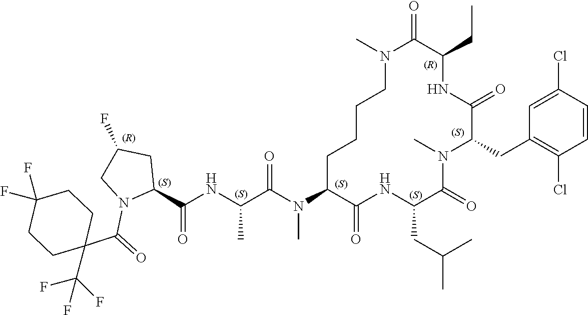 |
| 524 | 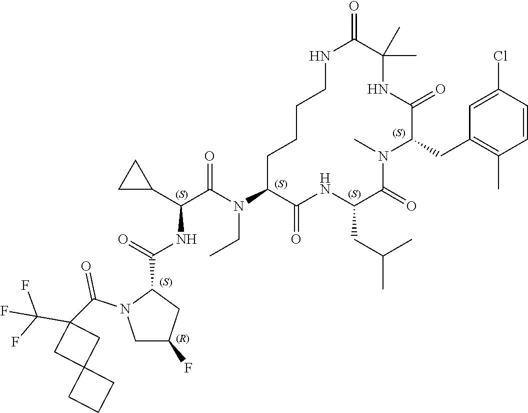 |
| 525 | 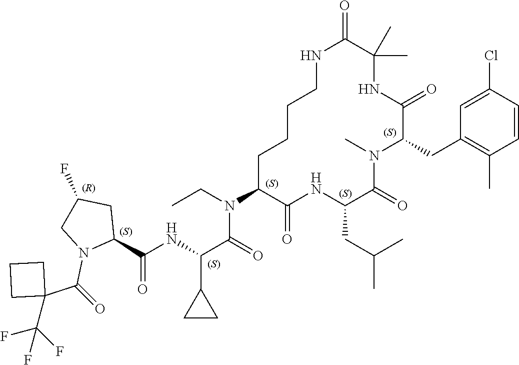 |
| 526 | 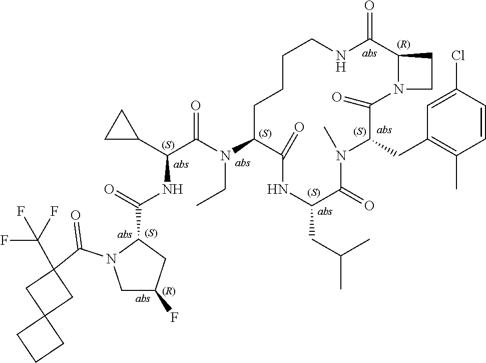 |
| 527 | 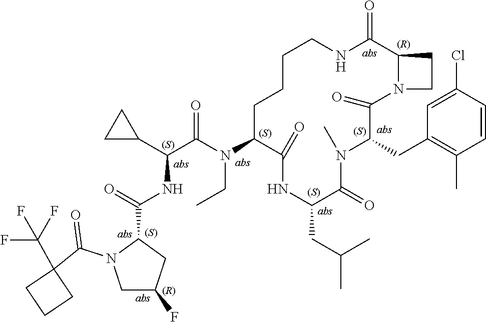 |
| 528 | 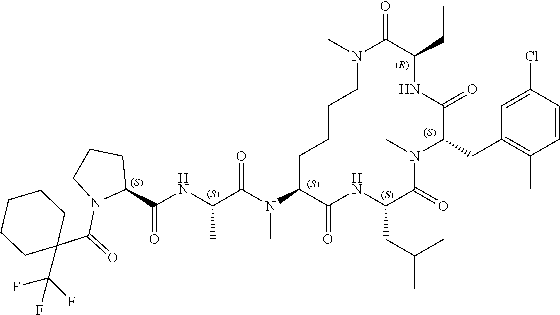 |
| 529 | 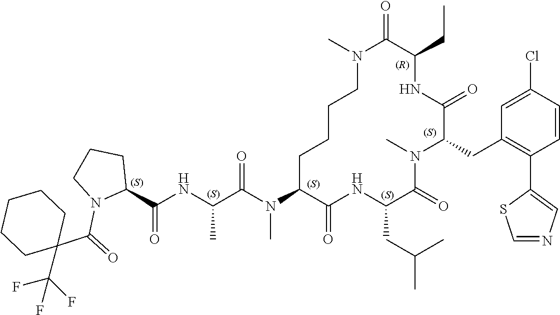 |
| 530 | 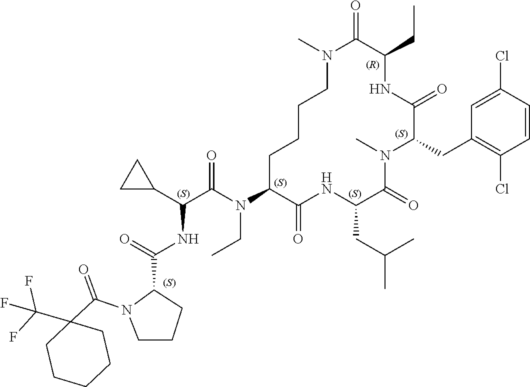 |
| 531 | 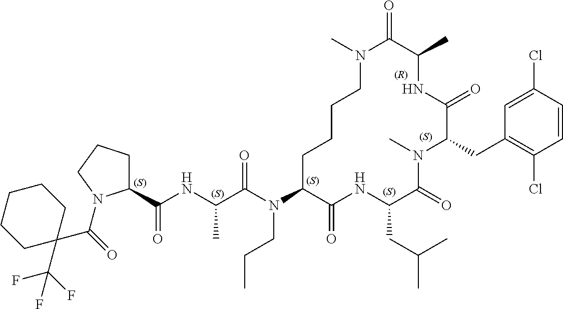 |
| 532 | 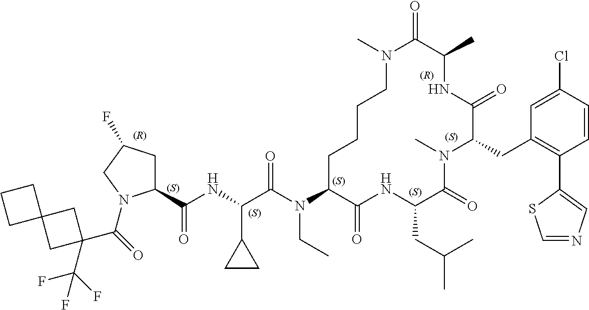 |
| 533 | 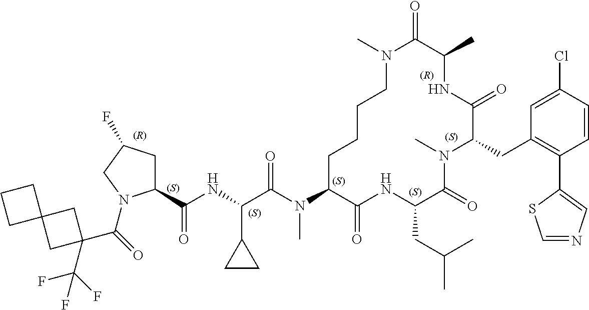 |
| 534 | 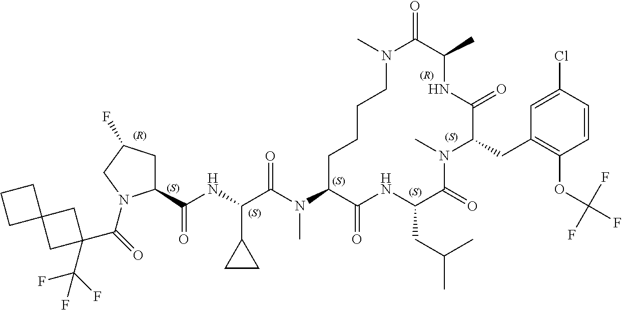 |
| 535 | 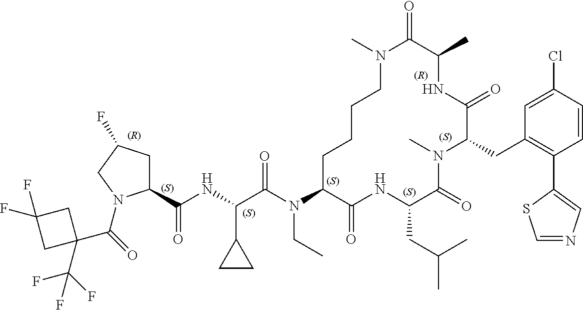 |
| 536 | 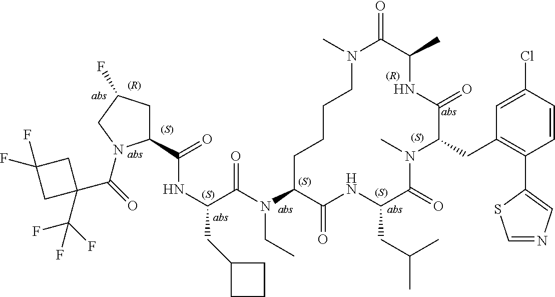 |
| 537 | 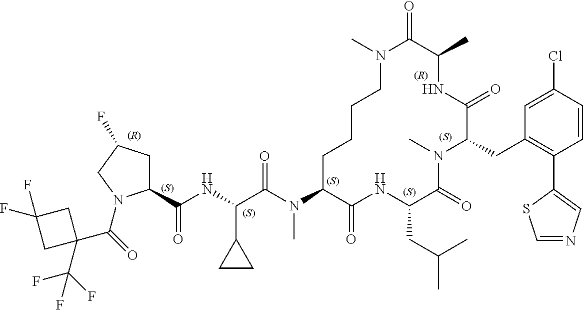 |
| 538 | 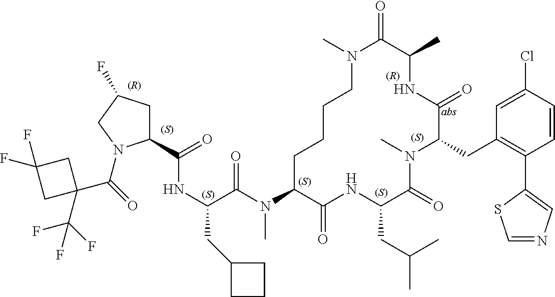 |
| 539 | 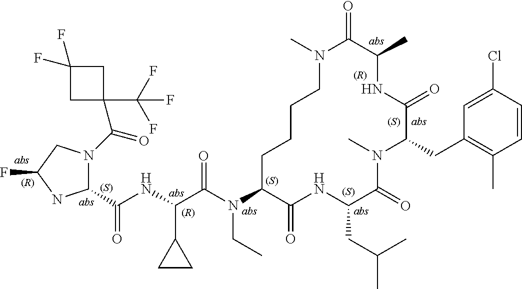 |
| 540 | 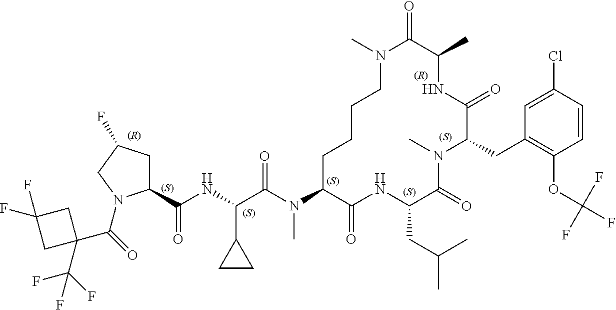 |
| 541 | 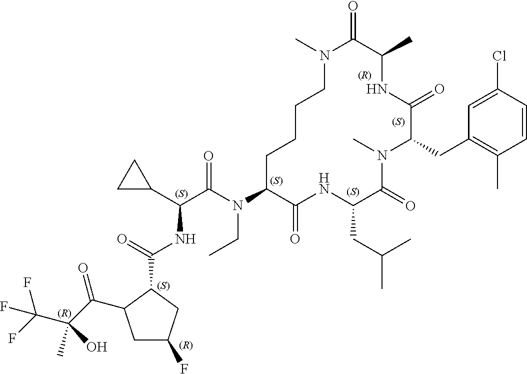 |
| 542 | 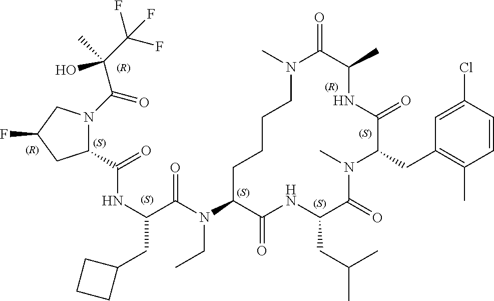 |
| 543 | 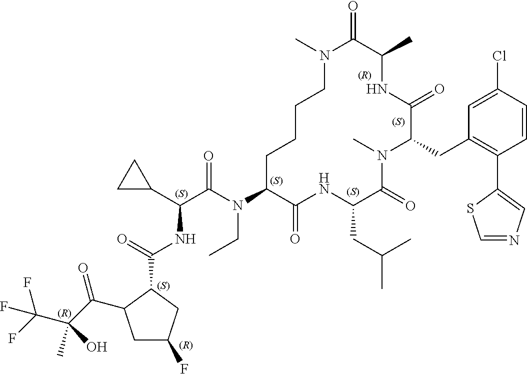 |
| 544 | 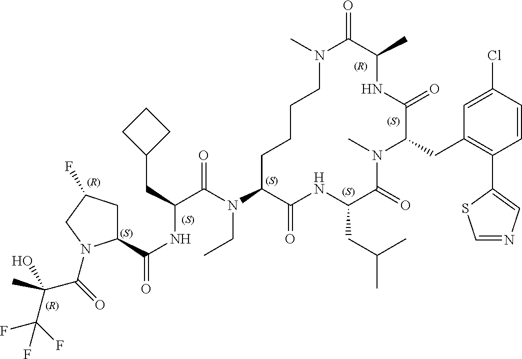 |
| 545 | 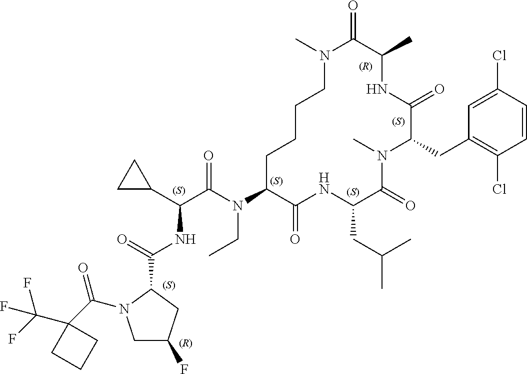 |
| 546 | 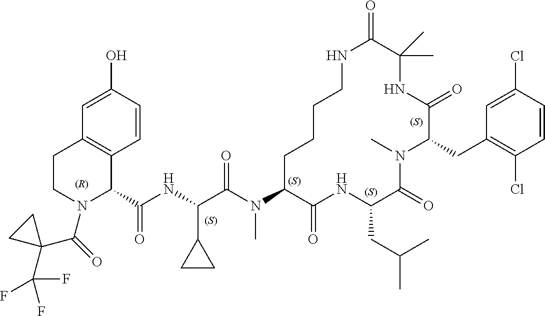 |
| 547 | 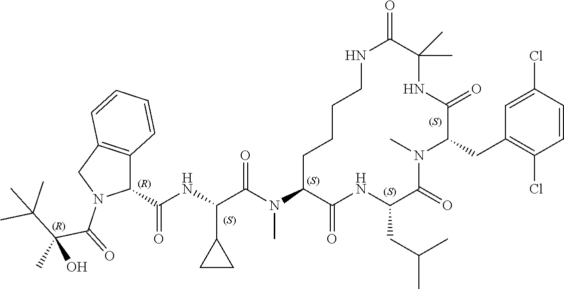 |
| 548 | 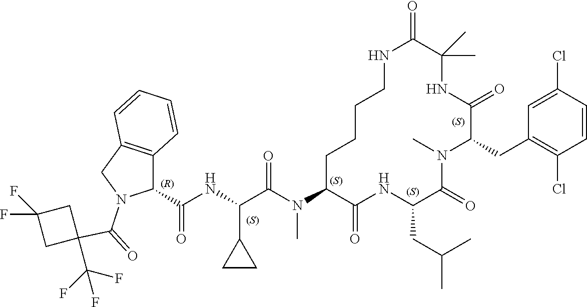 |
| 549 | 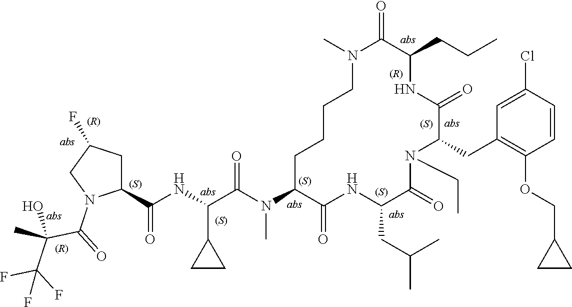 |
| 550 | 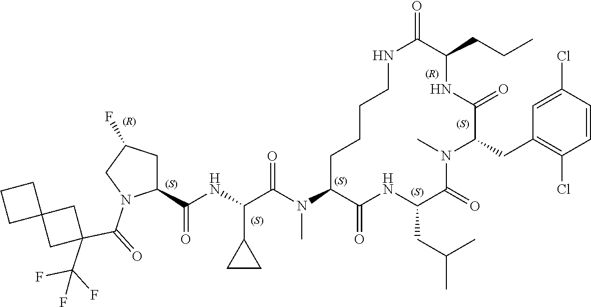 |
| 551 | 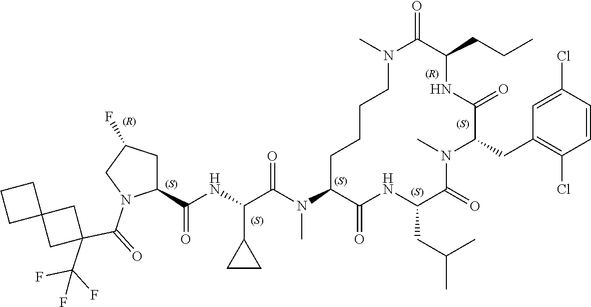 |
| 552 | 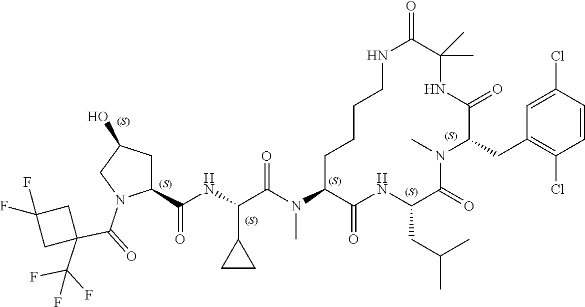 |
| 553 | 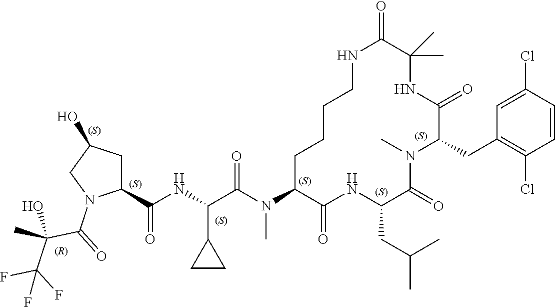 |
| 554 | 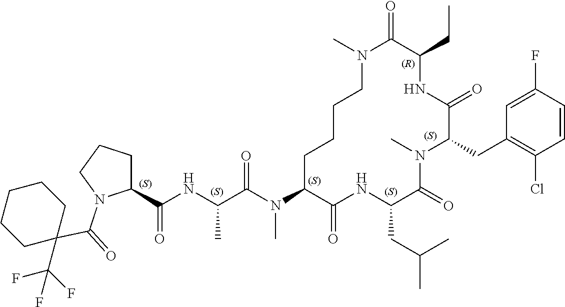 |
| 555 | 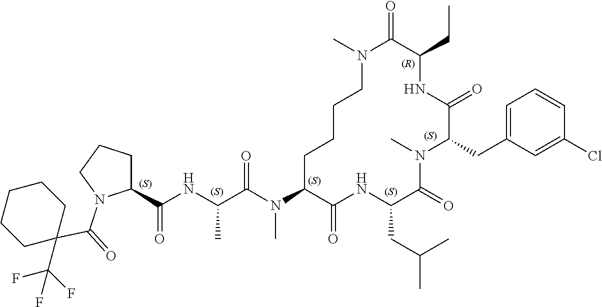 |
| 556 | 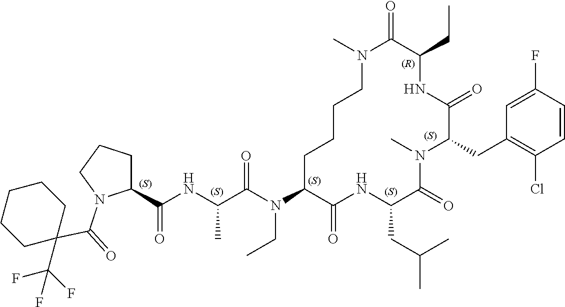 |
| 557 | 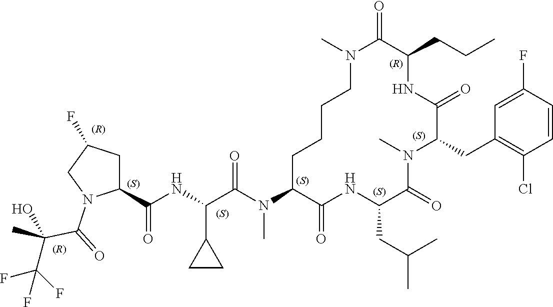 |
| 558 | 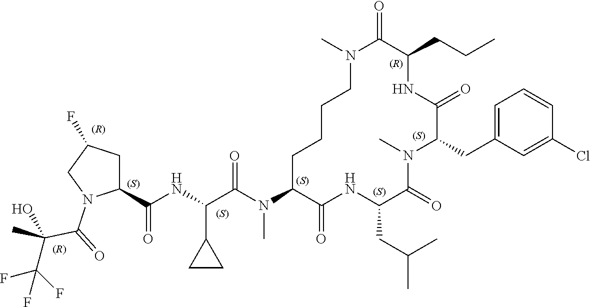 |
| 559 | 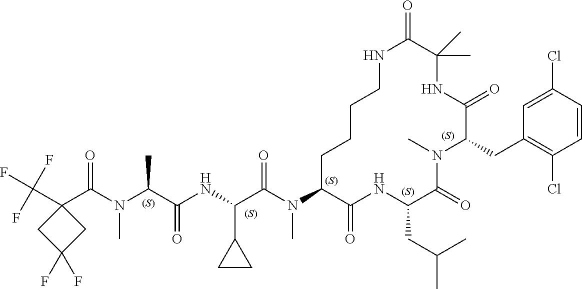 |
| 560 | 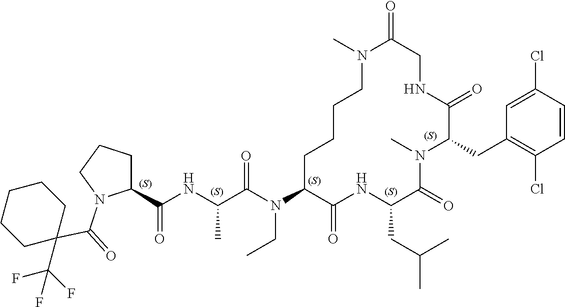 |
| 561 | 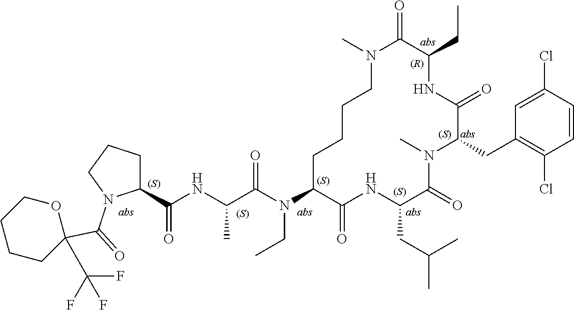 |
| 562 | 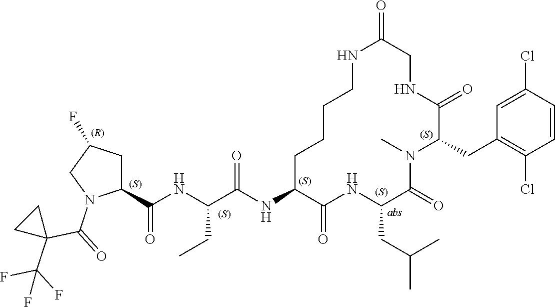 |
| 563 | 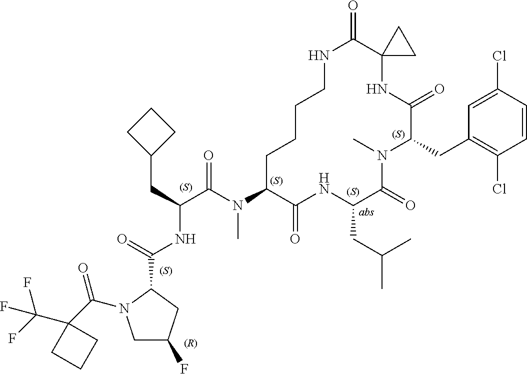 |
| 564 | 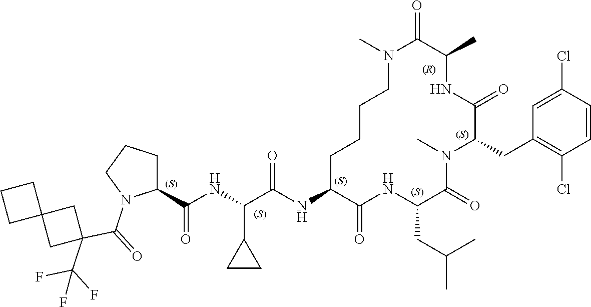 |
| 565 | 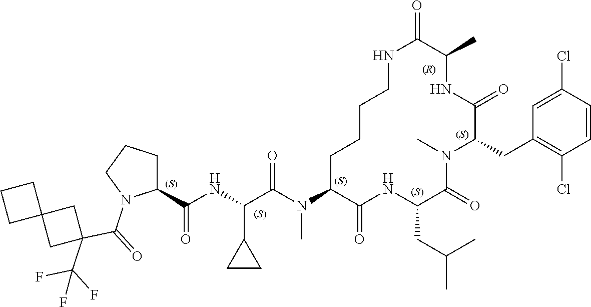 |
| 566 | 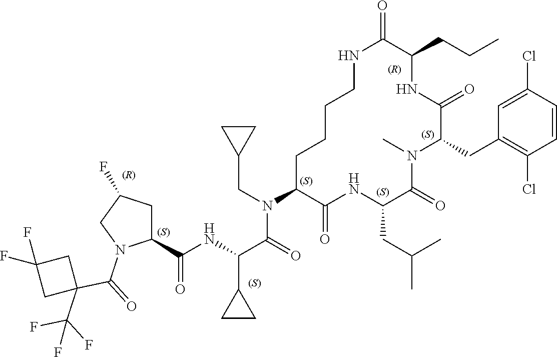 |
| 567 | 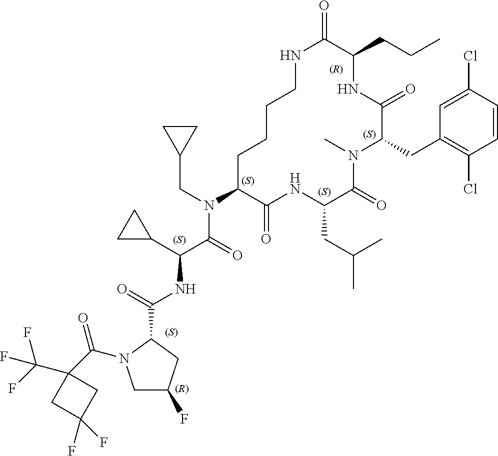 |
| 568 | 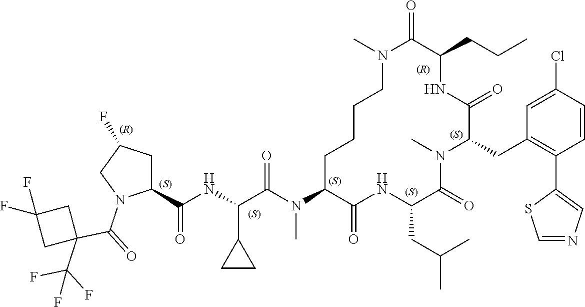 |
| 569 | 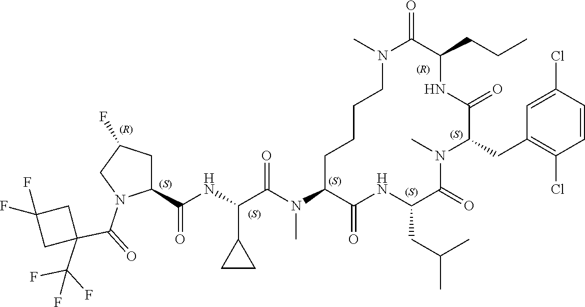 |
| 570 | 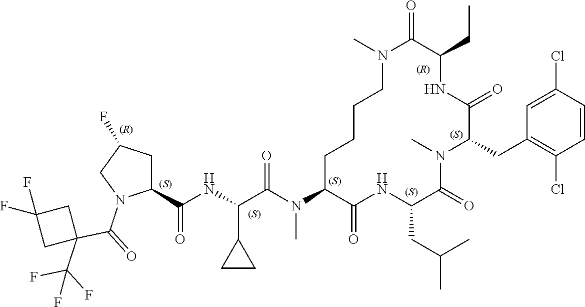 |
| 571 | 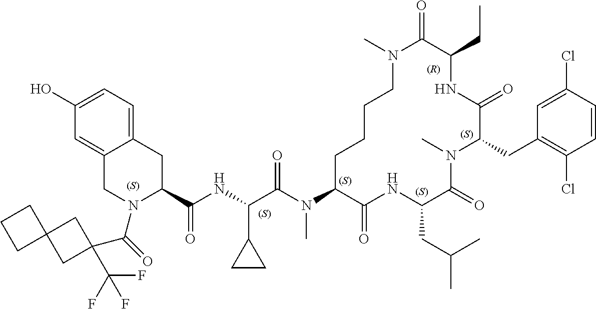 |
| 572 | 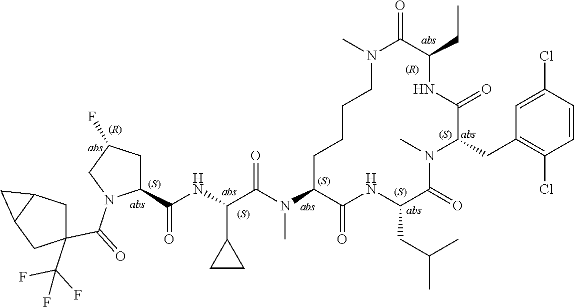 |
| 573 | 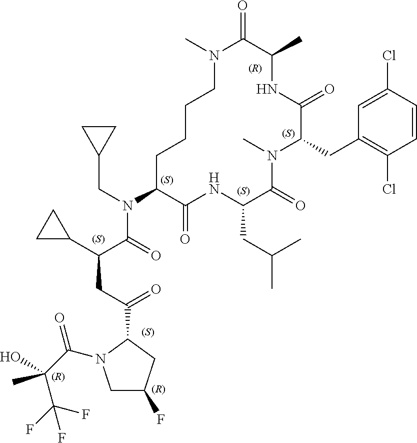 |
| 574 | 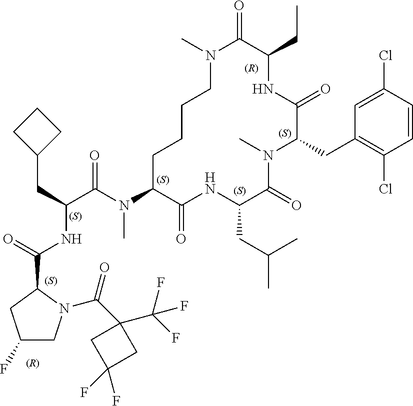 |
| 575 | 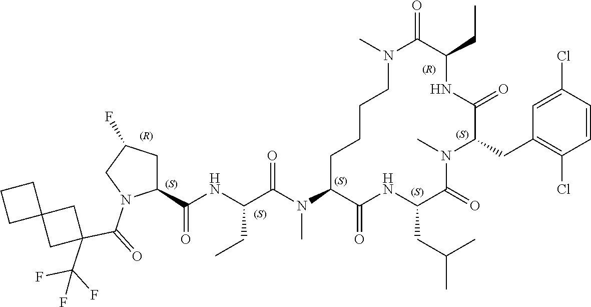 |
| 576 | 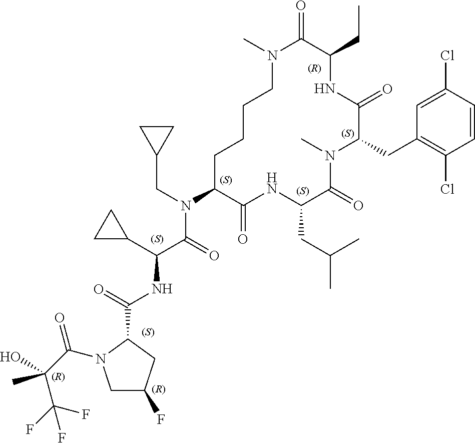 |
| 577 | 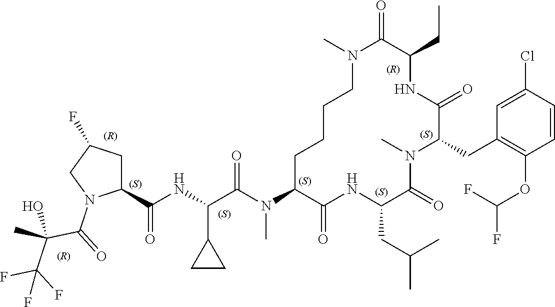 |
| 578 | 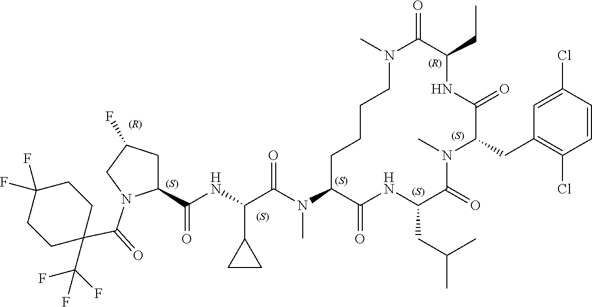 |
| 579 | 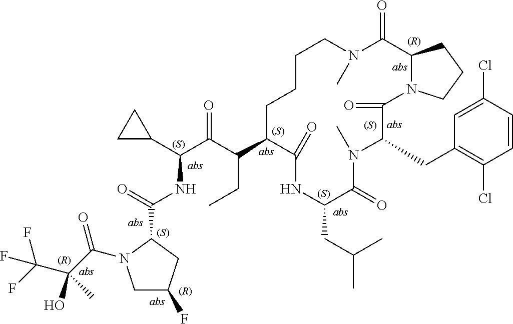 |
| 580 | 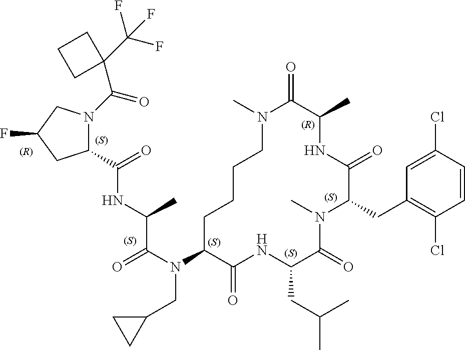 |
| 581 | 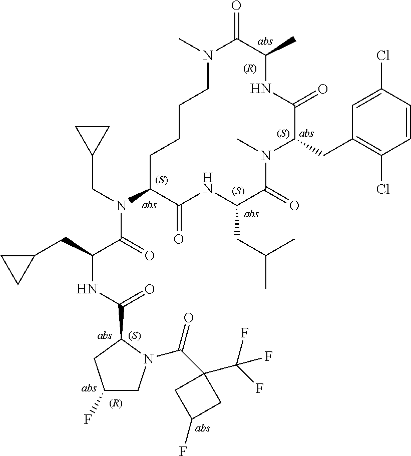 |
| 582 | 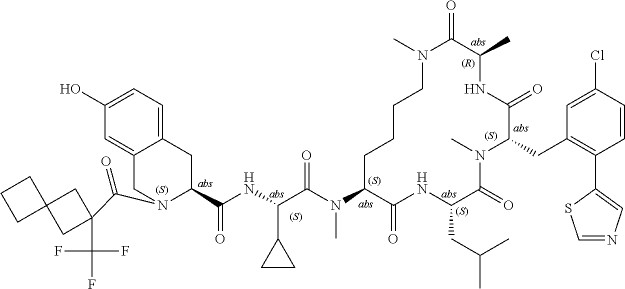 |
| 583 | 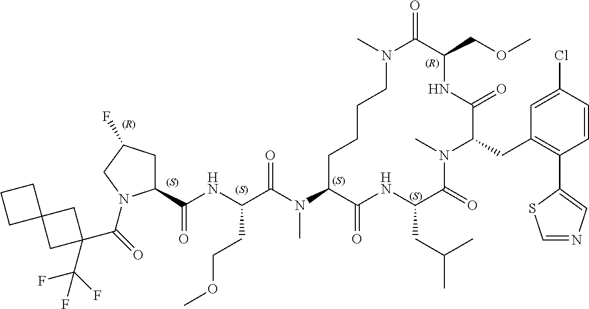 |
| 584 | 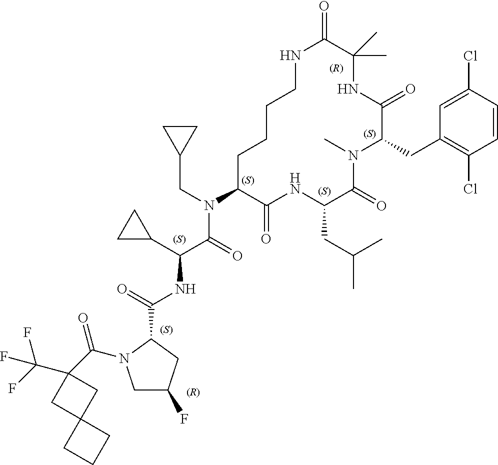 |
| 585 | 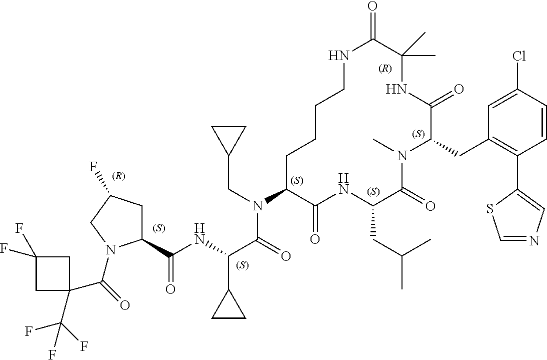 |
| 586 | 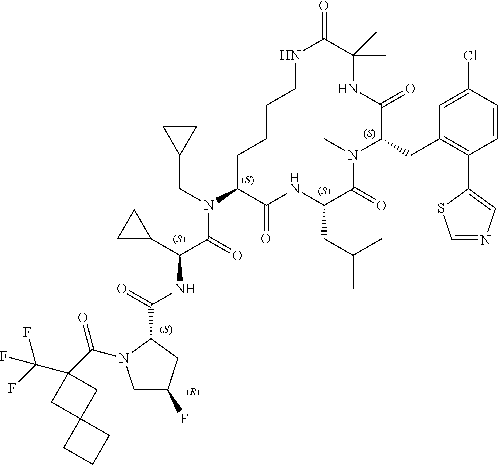 |
| 587 | 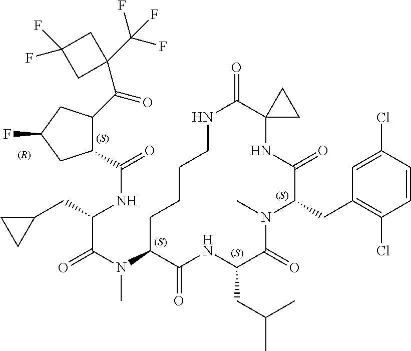 |
| 588 | 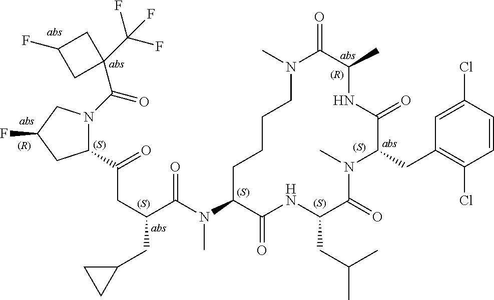 |
| 589 | 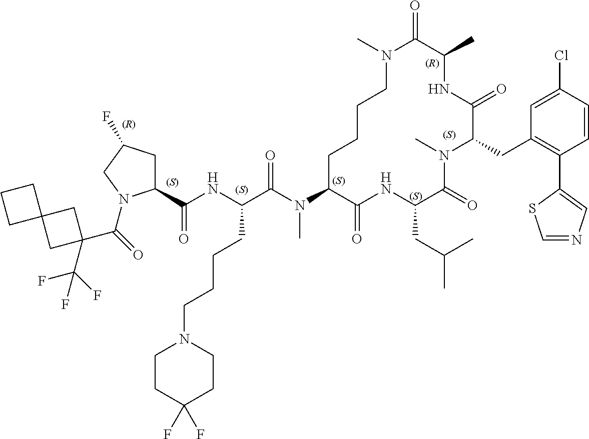 |
| 590 | 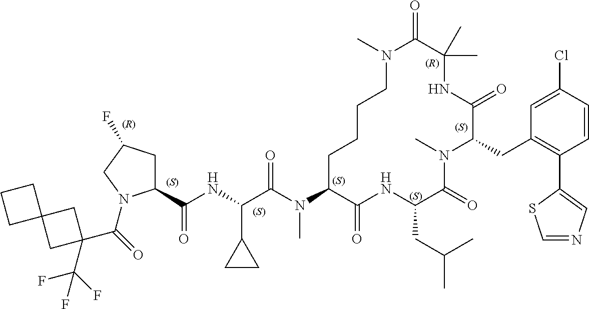 |
| 591 | 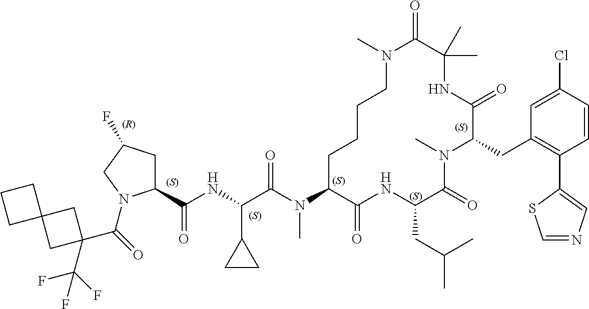 |
| 592 | 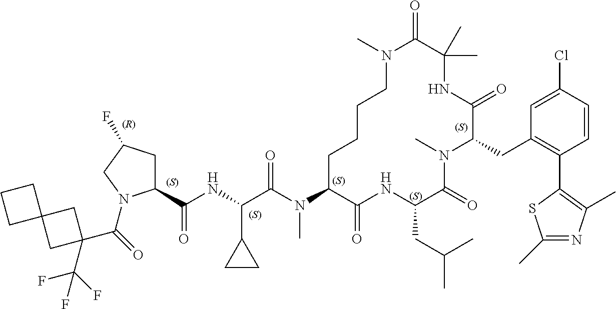 |
| 593 | 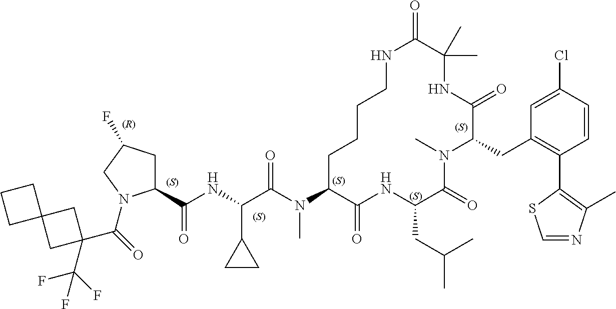 |
| 594 | 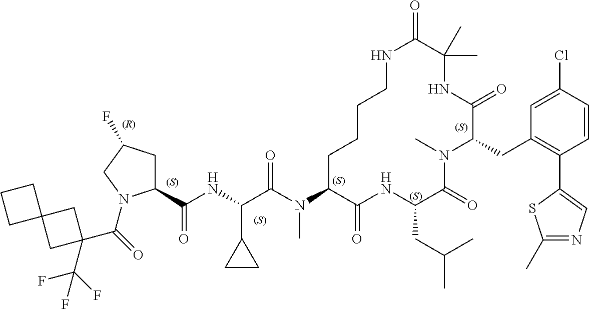 |
| 595 | 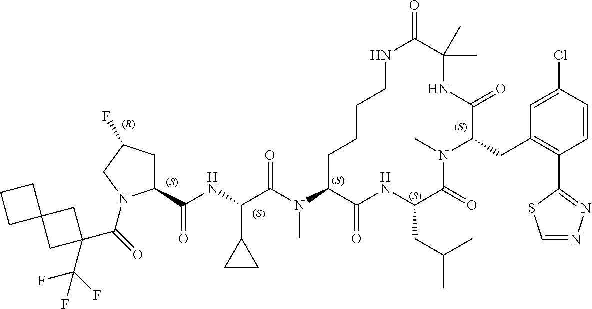 |
| 596 | 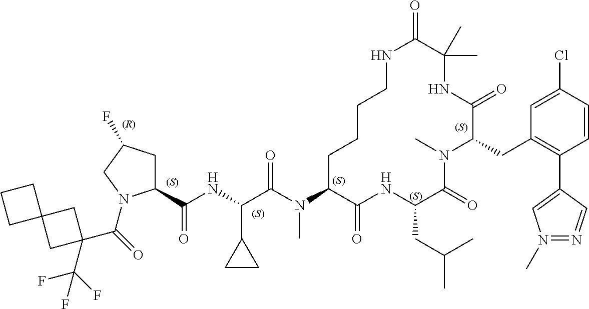 |
| 597 | 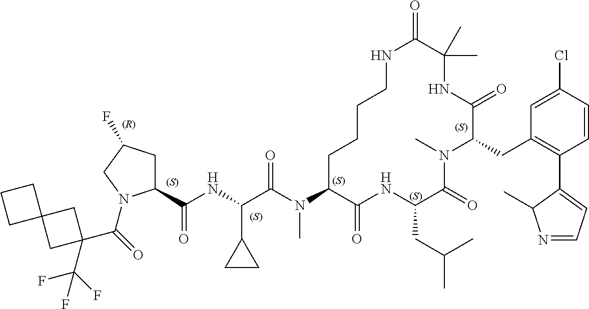 |
| 598 | 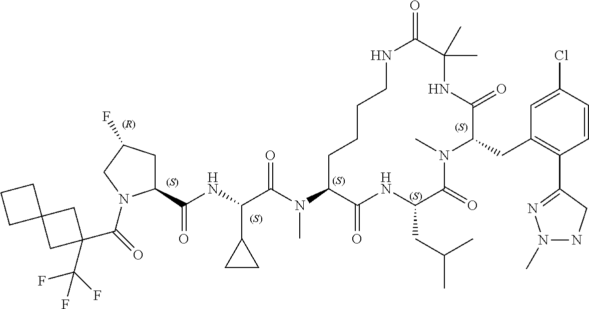 |
| 599 | 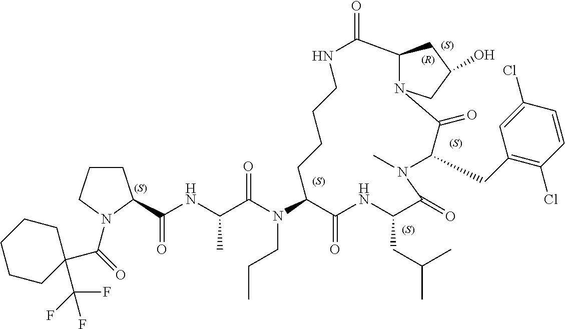 |
| 600 | 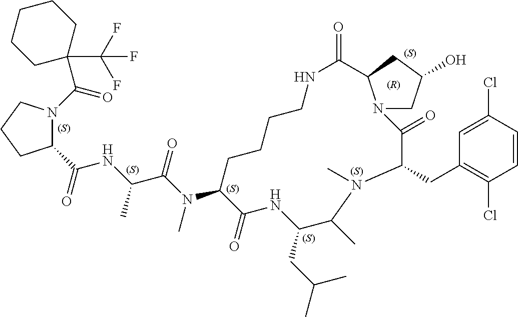 |
| 601 | 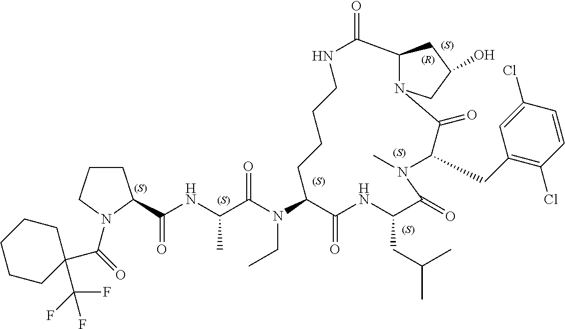 |
| 602 | 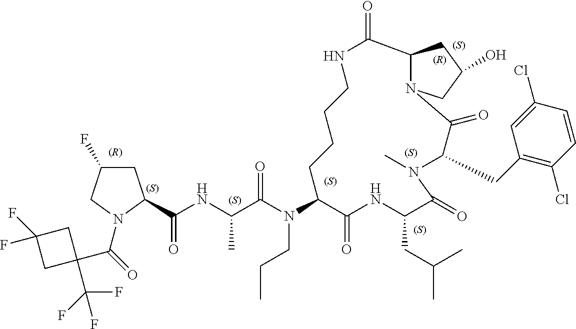 |
| 603 | 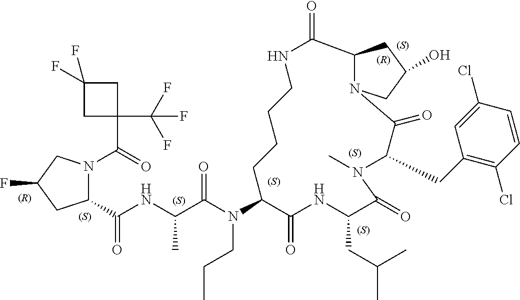 |
| 604 | 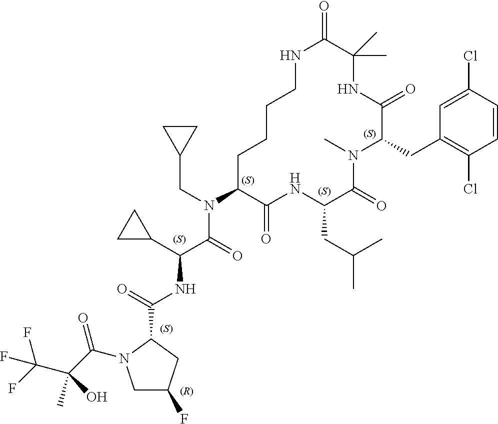 |
| 605 | 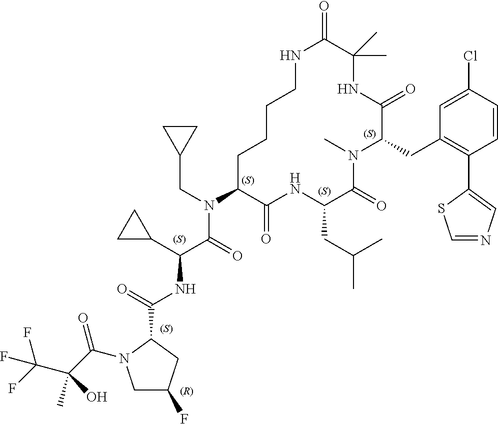 |
| 606 | 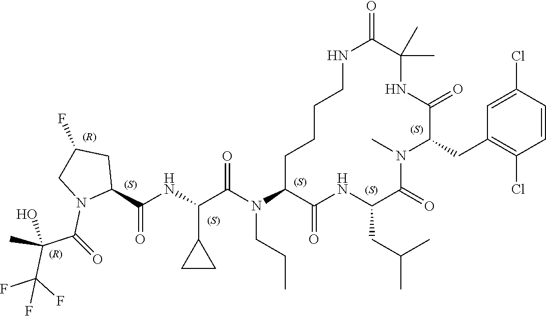 |
| 607 | 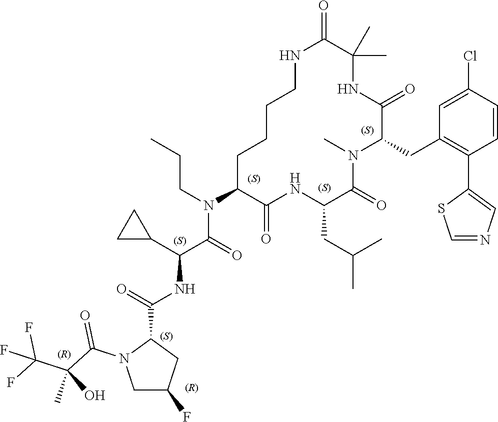 |
| 608 | 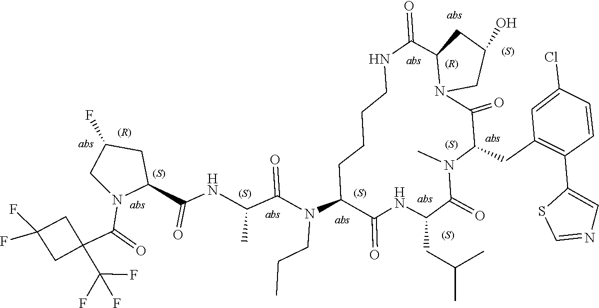 |
| 609 | 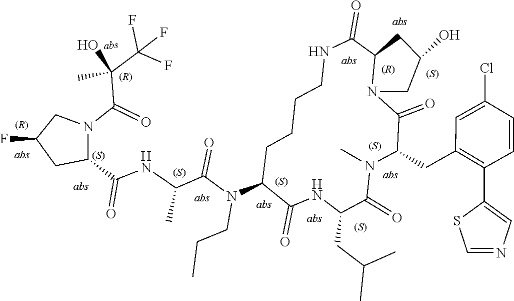 |
| 610 | 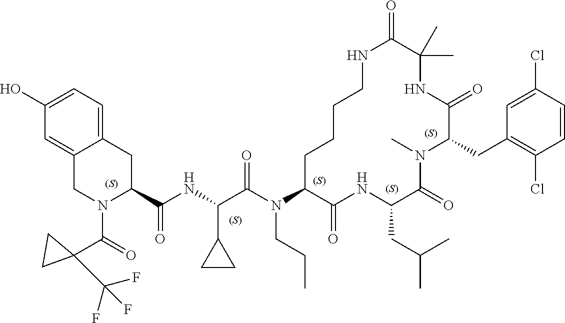 |
| 611 | 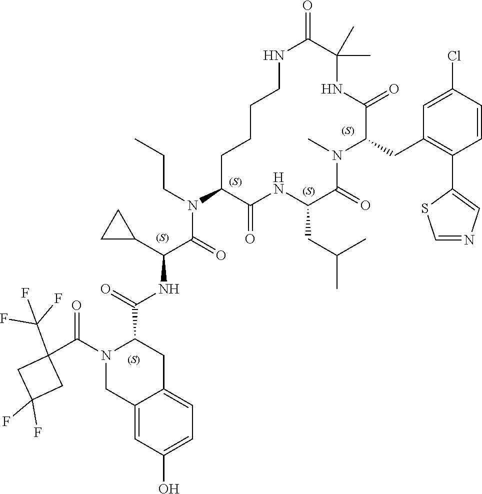 |
| 612 | 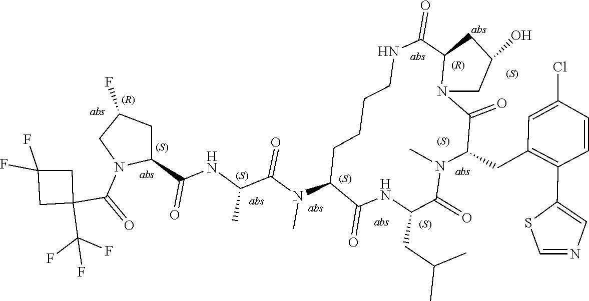 |
| 613 | 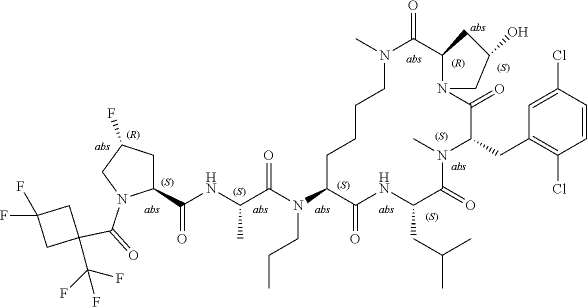 |
| 614 | 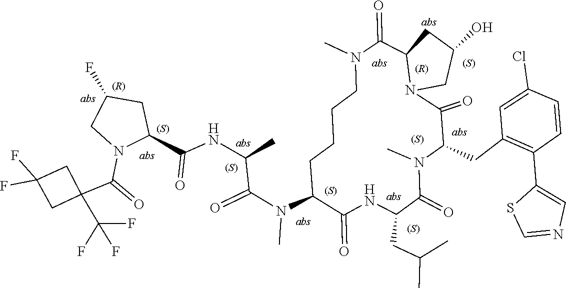 |
| 615 | 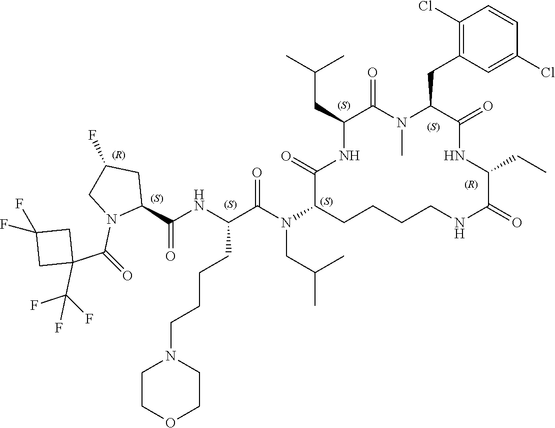 |
| 616 | 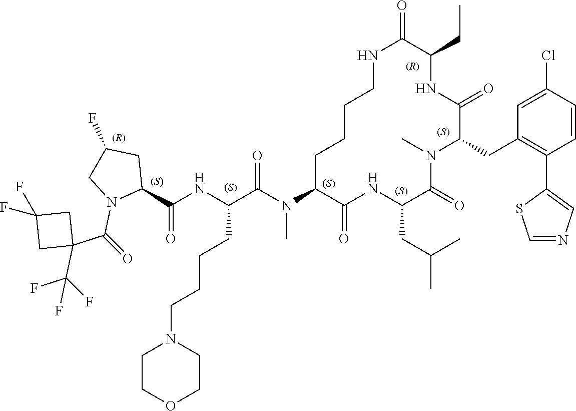 |
| 617 | 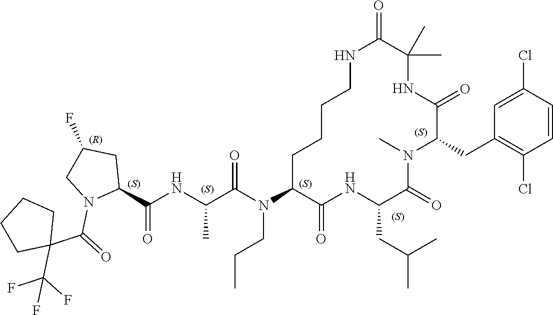 |
| 618 | 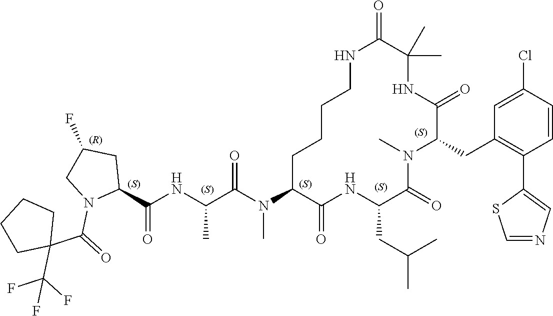 |
| 619 | 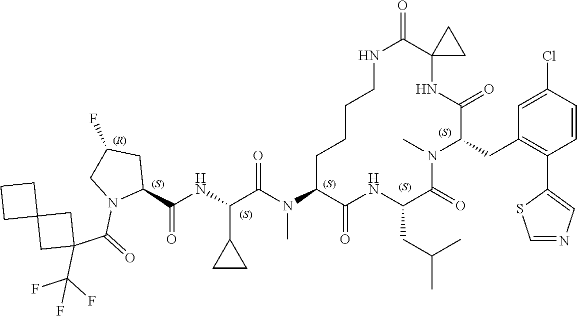 |
| 620 | 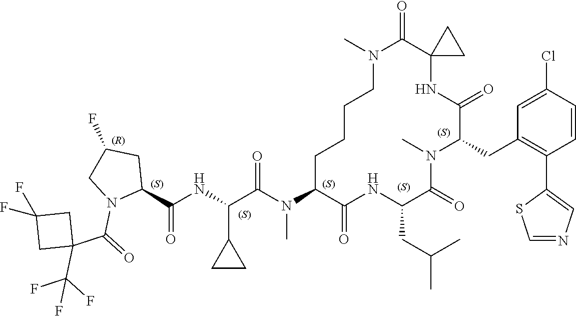 |
| 621 | 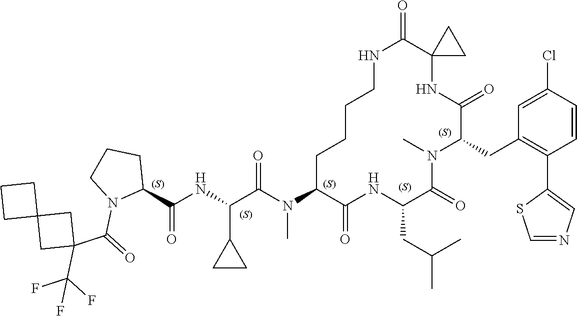 |
| 622 | 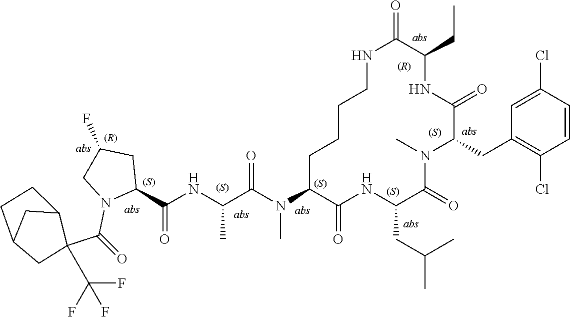 |
| 623 | 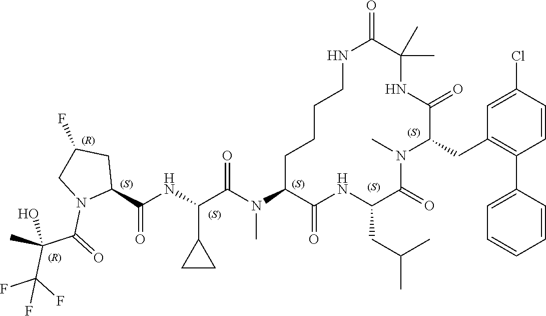 |
| 624 | 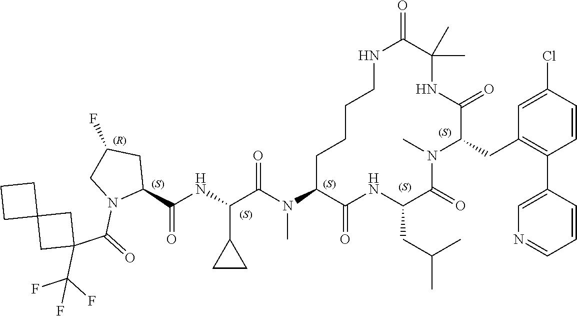 |
| 625 | 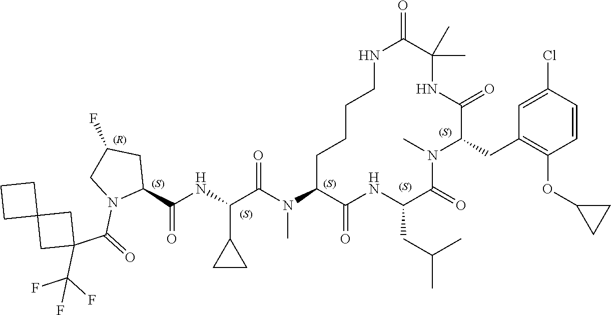 |
| 626 | 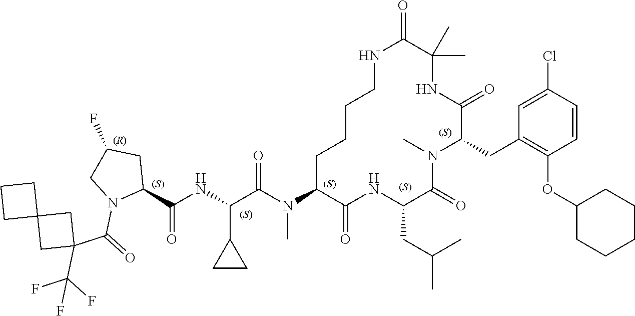 |
| 627 | 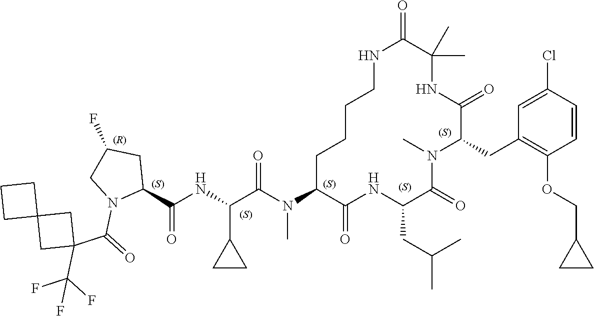 |
| 628 | 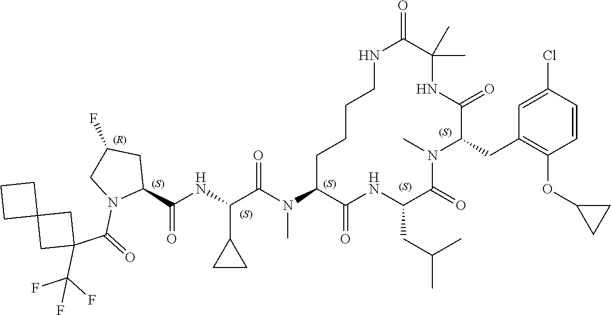 |
| 629 | 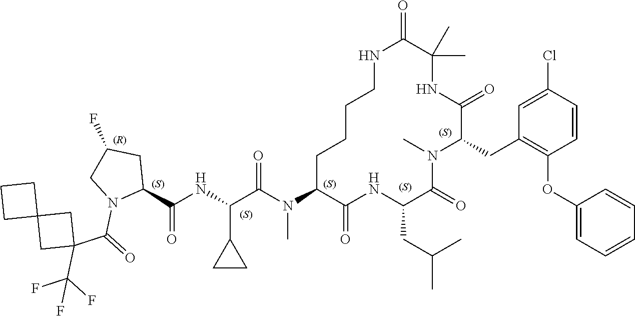 |
| 630 | 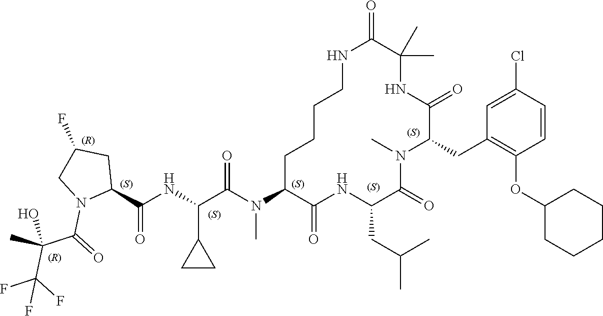 |
| 631 | 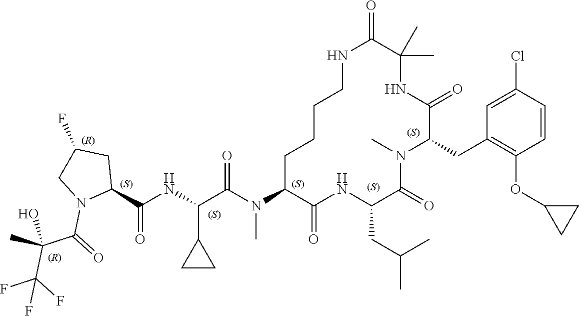 |
| 632 | 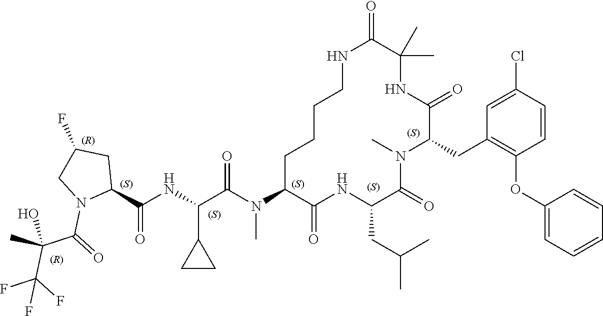 |
| 633 | 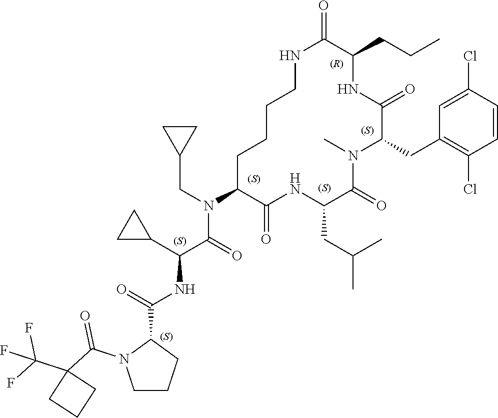 |
| 634 | 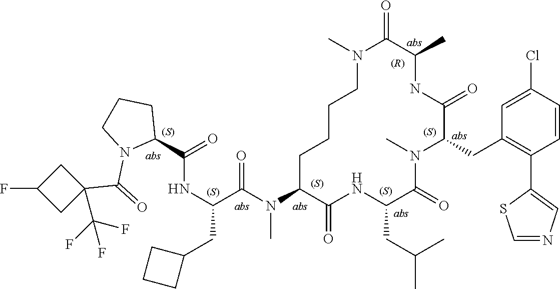 |
| 635 | 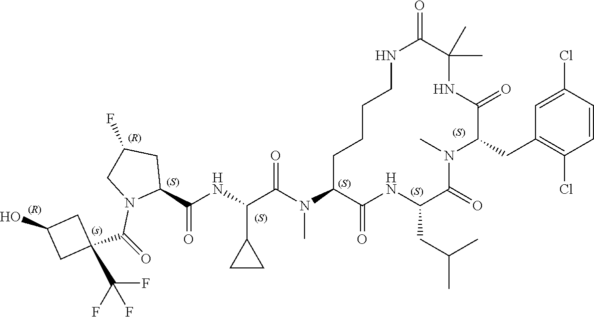 |
| 636 | 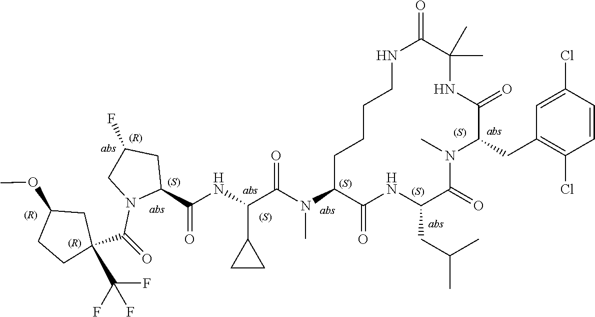 |
| 637 | 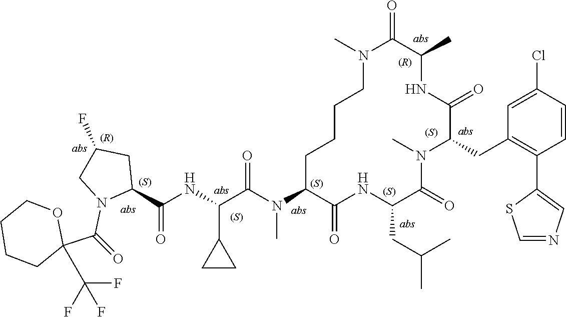 |
| 638 | 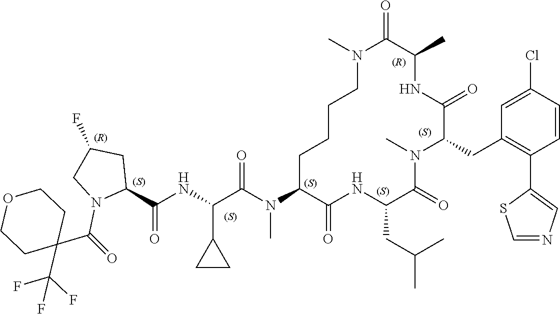 |
| 639 | 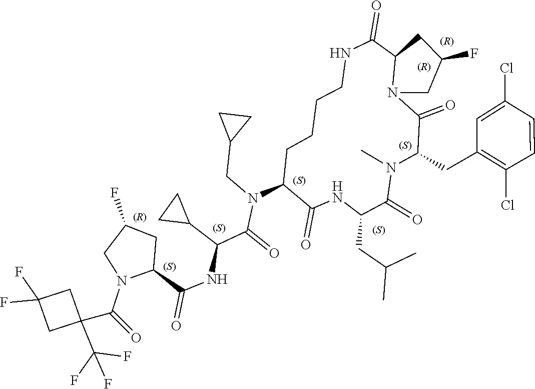 |
| 640 | 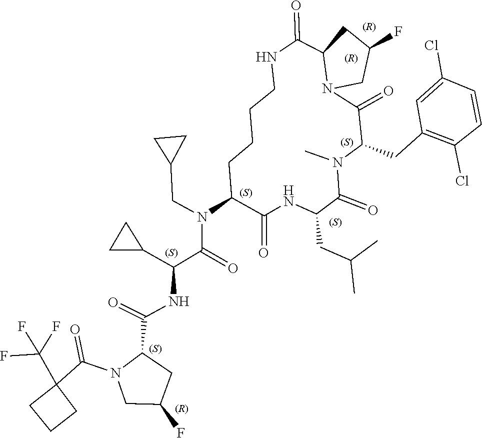 |
| 641 | 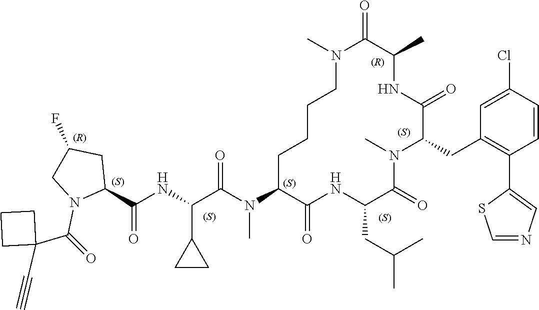 |
| 642 | 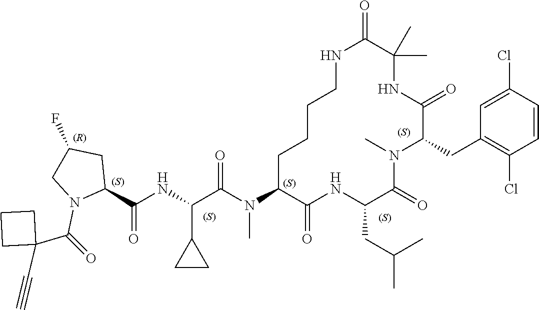 |
| 643 | 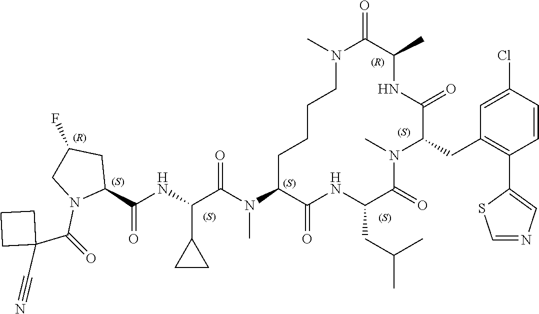 |
| 644 | 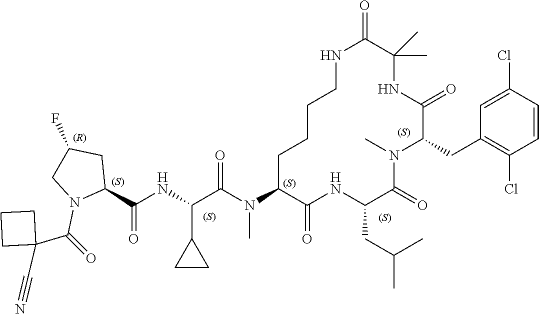 |
| 645 | 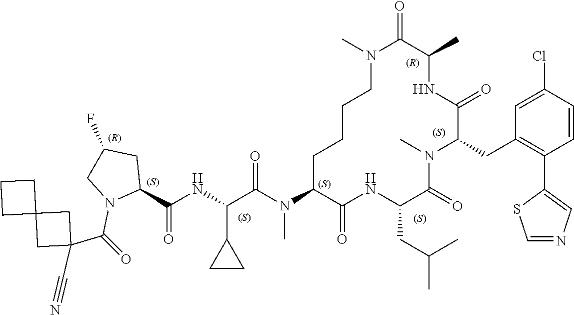 |
| 646 | 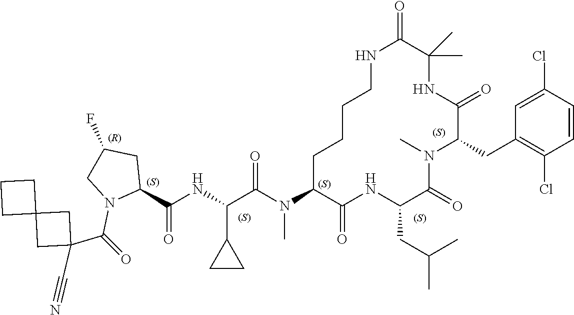 |
| 647 | 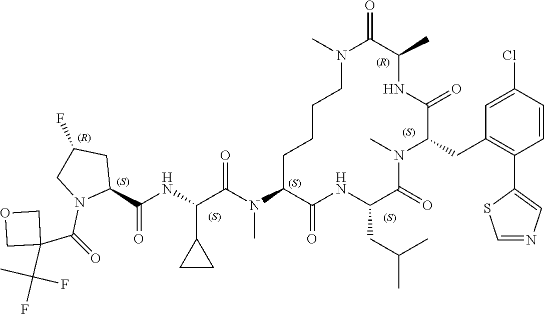 |
| 648 | 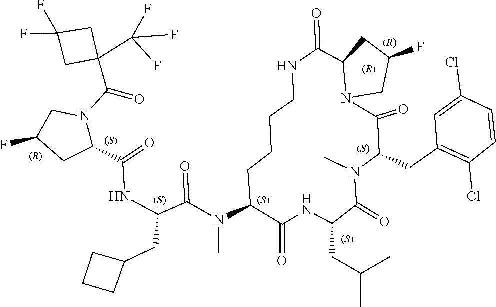 |
| 649 | 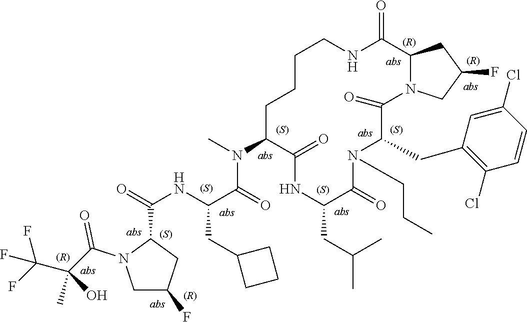 |
| 650 | 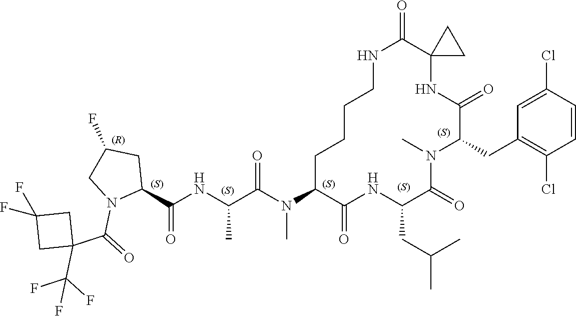 |
| 651 | 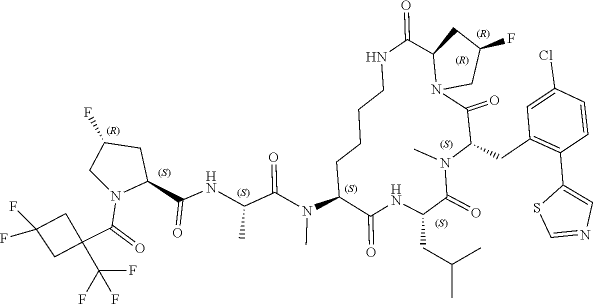 |
| 652 | 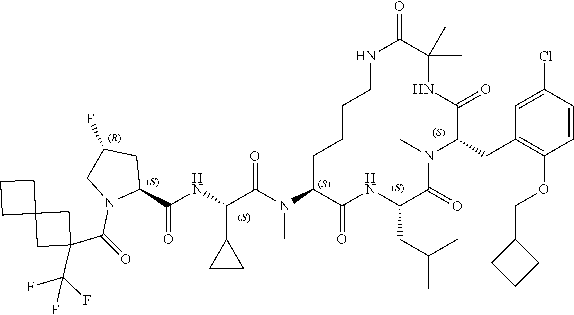 |
| 653 | 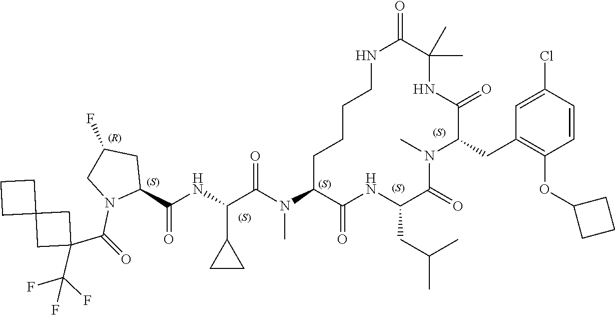 |
| 654 | 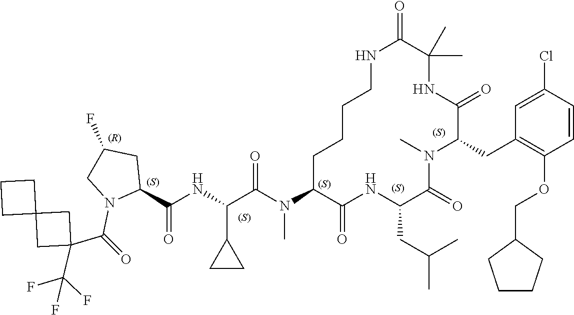 |
| 655 | 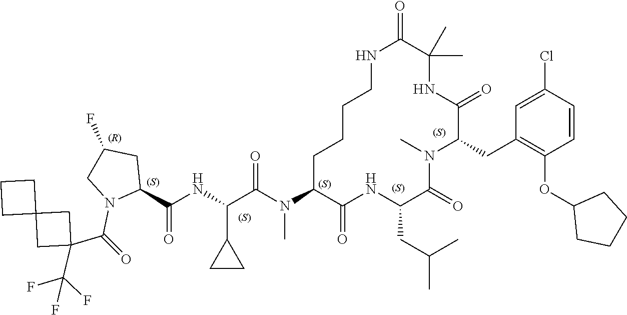 |
| 656 | 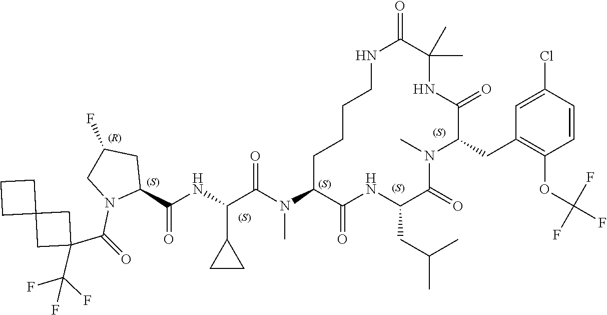 |
| 657 | 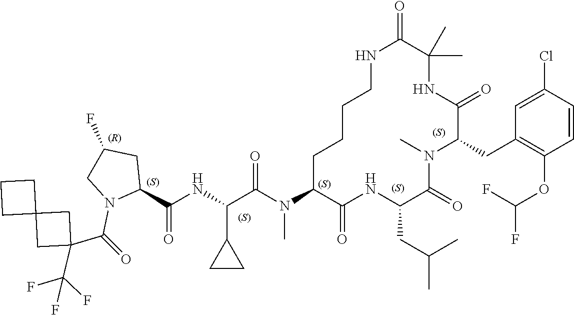 |
| 658 | 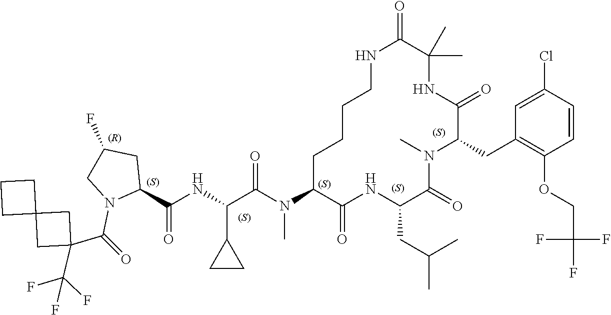 |
| 659 | 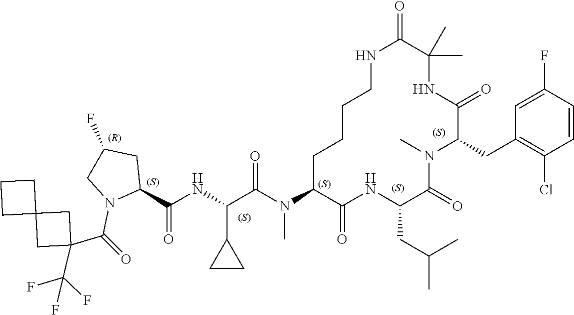 |
| 660 | 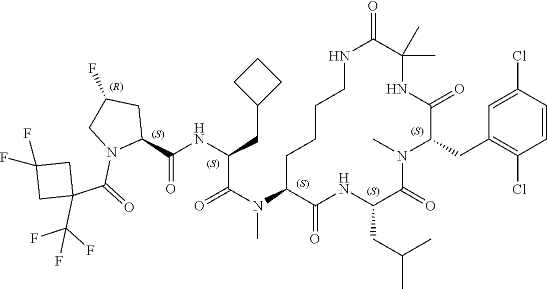 |
| 661 | 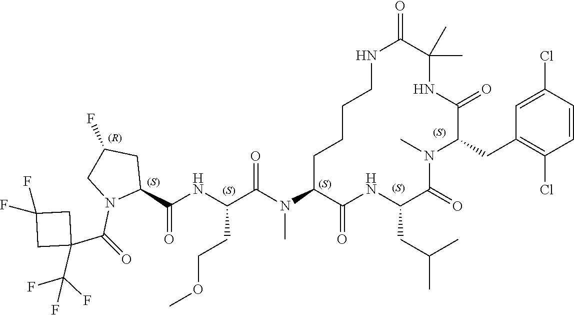 |
| 662 | 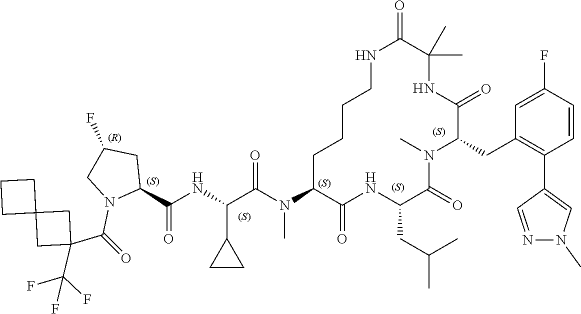 |
| 663 | 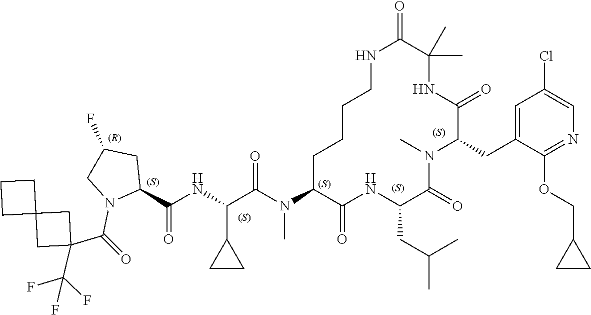 |
| 664 | 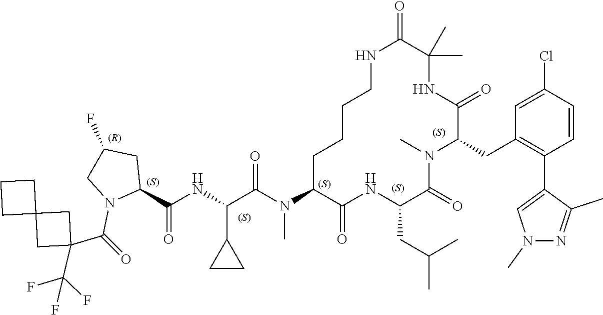 |
| 665 | 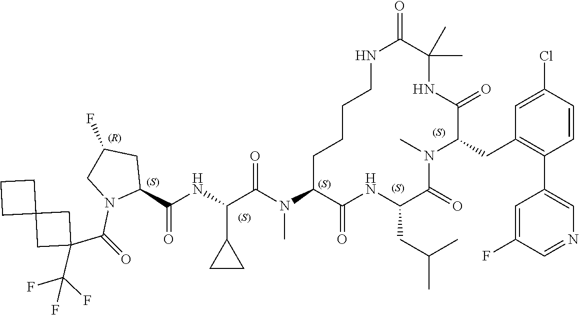 |
| 666 | 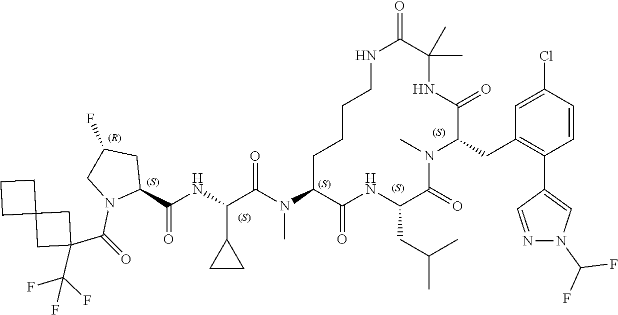 |
| 667 | 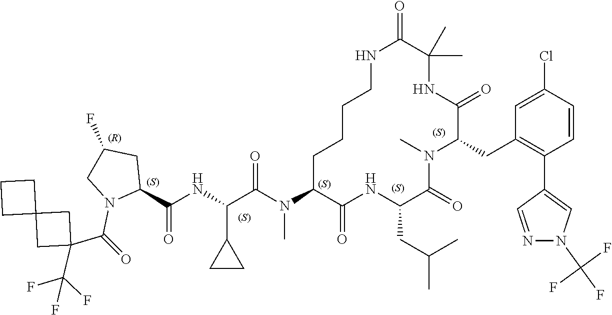 |
| 668 | 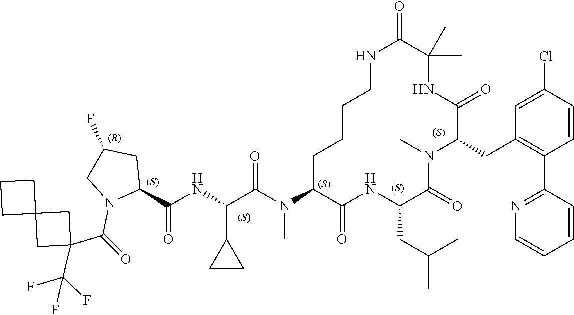 |
| 669 | 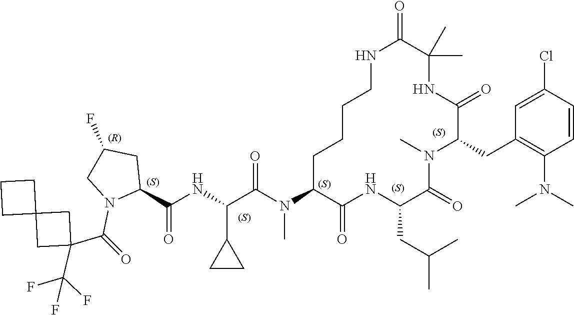 |
| 670 | 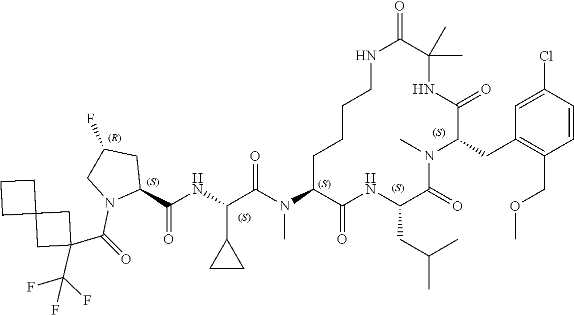 |
| 671 | 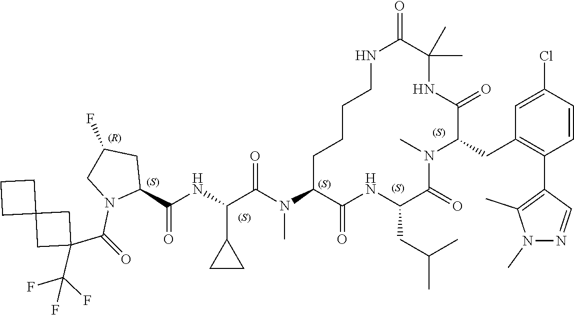 |
| 672 | 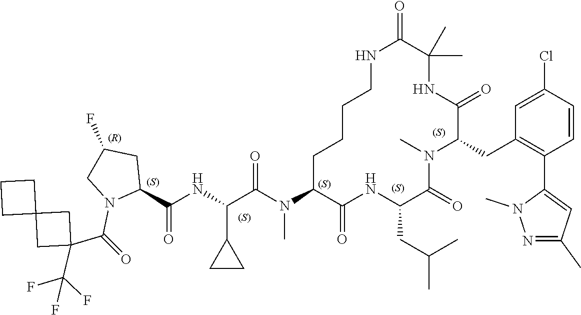 |
| 673 | 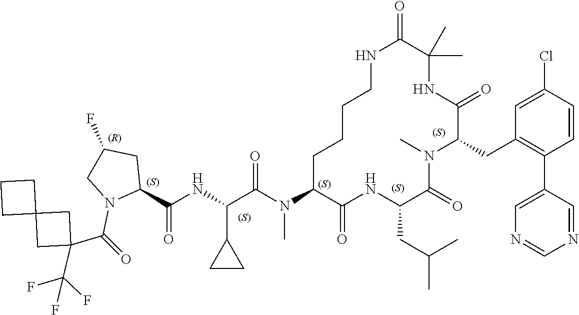 |
| 674 | 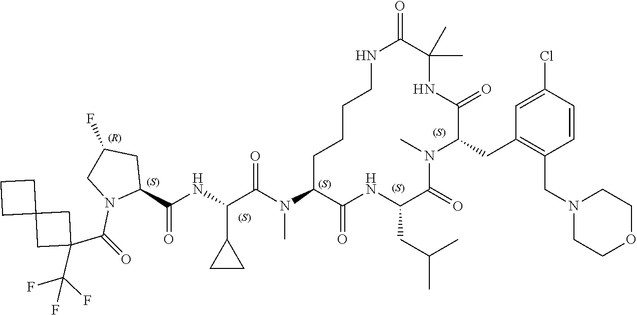 |
| 675 | 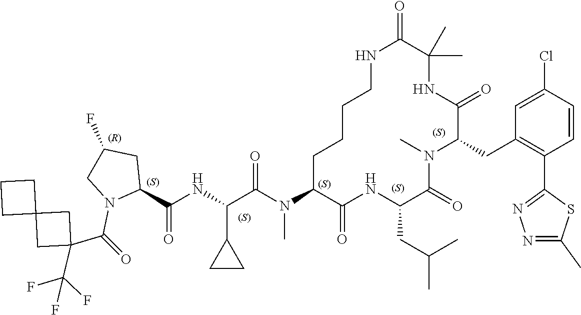 |
| 676 | 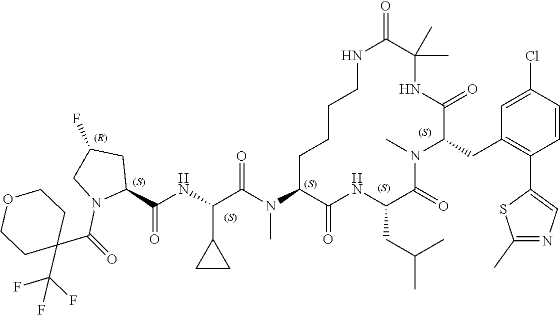 |
| 677 | 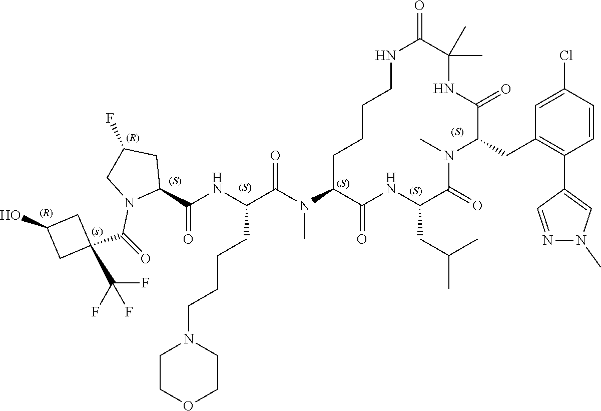 |
| 678 | 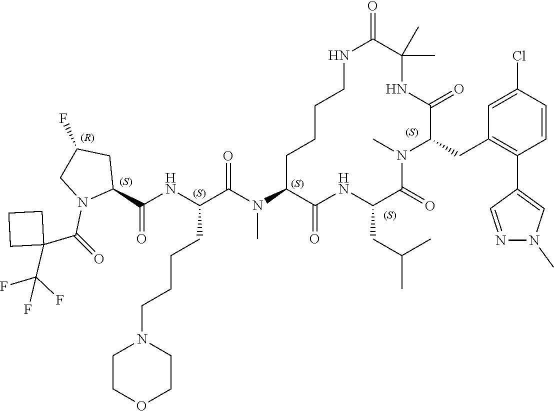 |
| 679 | 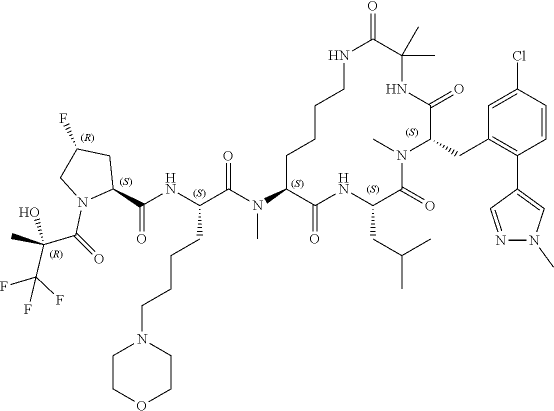 |
| 680 | 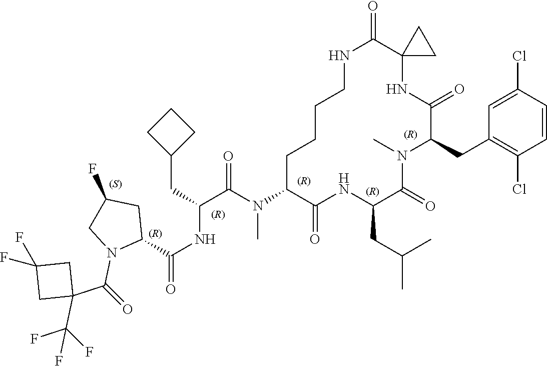 |

### C. Solution Synthesis Methods

Certain compounds of Formula I described herein were not prepared by adding all Building Blocks via linear solid phase synthesis, cleaving, and then cyclizing. Some of the compounds prepared herein include post cyclization modifications, or are partially synthesized using linear solid phase synthesis, cleaved and then have further manipulations in solution such as adding additional Building Blocks or additional chemical modifications.

### 1. Synthesis of Exemplary Cyclic Intermediate for Further Modification

The reaction diagram and paragraphs below describe linear peptide synthesis and cyclization of an intermediate, UX-0066, that is used for further modifications (Suzuki couplings) and building block additions to the N-terminal amine. It is understood that this is an exemplary intermediate and that the exact chemical structure can be modified without departing from the teachings herein.

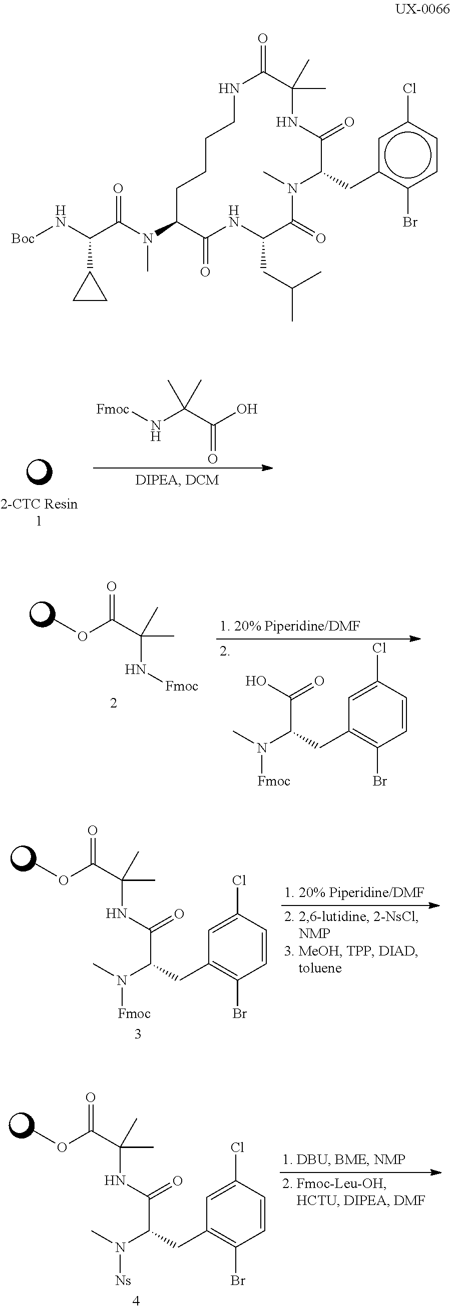

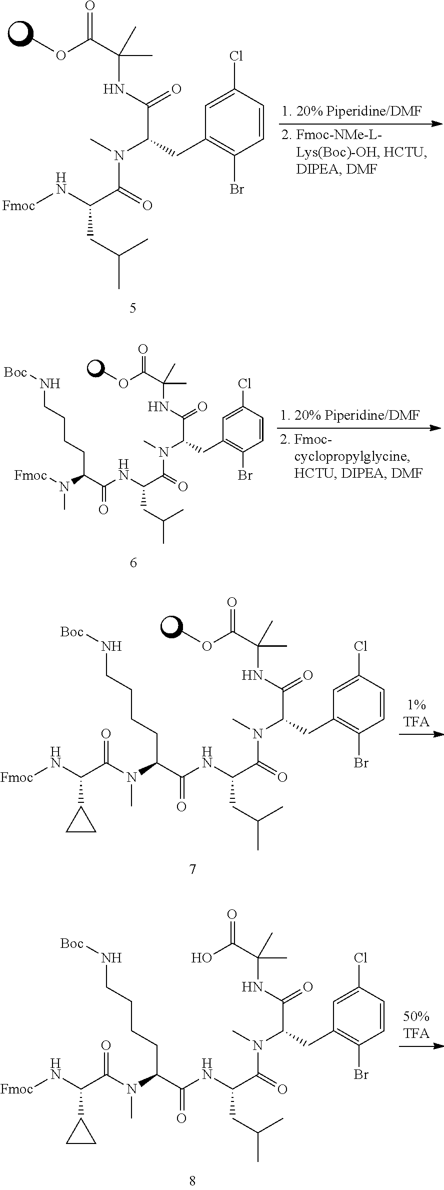

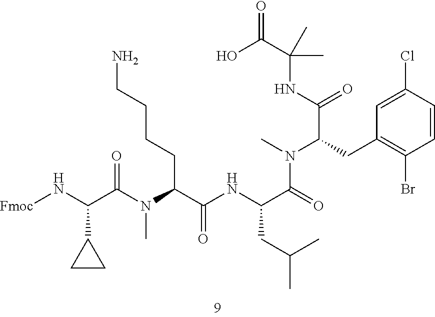

### Transformation 1 to 2:

The 2-Chlorotrityl chloride resin (22.8 g, 25.63 mmol, 1.12 mmol/g loading capacity) was added to the glass vessel, then DCM (200 mL) was added and agitated under nitrogen for 30 min. The solution was drained and DCM (100 mL) was added to the resin. Then a solution of compound 1 (10 g, 30.74 mmol, 1.2 eq) and DIPEA (9.93 g, 76.84 mmol, 13.38 mL, 3 eq) in DCM (250 mL) was added to the above resin. The mixture was kept at room temperature for 8 h while a stream of nitrogen was bubbled through it. The resulting suspension was drained through a filter and discarded. The resin was washed with DCM (250 mL*3) and the loading step was repeated twice. Then the resin was washed in turned 3 times with MeOH (250 mL), DMF (250 mL).

### Transformation 2 to 3:

20% piperidine in DMF (200 mL*6) was added to the resin and agitated under nitrogen for 10 minutes. The solution was drained and the and the deprotection was repeated three additional times. The peptidyl resin was washed with DMF (200 mL*8) at room temperature. The loading level of the resin was determined to be 0.94 mmol/g via standard UV absorption method.

To a stirred solution of (2S)-3-(2-bromo-5-chloro-phenyl)-2-(9H-fluoren-9-ylmethoxycarbonylamino)propanoic acid (10.77 g, 21.51 mmol, 1 eq) in DMF (300 mL) was added HCTU (13.35 g, 32.27 mmol, 2 eq), DIPEA (8.34 g, 64.54 mmol, 11.24 mL, 3 eq) at 0° C. and the solution was stirred for 1 h at 0° C. under N₂. Then the solution was added to the above resin and permitted to react for 12 h at room temperature while a stream of nitrogen was bubbled through it. LCMS was used to indicate reaction completion. Upon completion, the peptidyl resin was drained and washed with DMF (200 mL*3).

### Transformation 3 to 4:

20% piperidine in DMF (200 mL) was added to the resin and permitted to react for 10 min under nitrogen. The solution was drained and the deprotection step was repeated six times in total. The resin was then washed with DMF (300 mL×8) at room temperature.

A solution of 2, 6-lutidine (17.76 g, 164.20 mmol, 19.30 mL, 10 eq) in NMP (150 mL) was added to the resin and was drummed for 5 min at room temperature with nitrogen. Then a solution of NsCl (5.49 g, 65.68 mmol, 4 eq) in toluene (150 mL) was added to the resin, the reaction was kept for 1 h while a stream of nitrogen was bubbled through it. The solution was drained. Then the resin was washed with DMF (200 mL×3), and the nosylation step was repeated once more.

A solution of triphenylphosphane (21.53 g, 82.10 mmol, 5 eq) in toluene (250 mL) was added to the resin, then DIAD (16.60 g, 82.10 mmol, 15.96 mL, 5 eq) was added dropwise to the resin (the temperature rose to 29° C.), and then MeOH (15.78 g, 492.60 mmol, 19.93 mL, 30 eq) was added dropwise to the resin (the temperature rose to 35° C.). The reaction was permitted to react for 1 h while a stream of nitrogen was bubbled through it. The resin was washed with DMF (200 mL×3), and the alkylation step was repeated a second time.

### Transformation 4 to 7:

DBU (12.44 g, 81.73 mmol, 12.32 mL, 5 eq) in NMP (300 mL) was added to the resin at room temperature. Then 2-sulfanylethanol (6.42 g, 82.17 mmol, 5.73 mL, 5.03 eq) was added dropwise to the resin, the reaction was permitted to proceed for 1 h while a stream of nitrogen was bubbled through it. The resin was washed with DMF (200 mL×3), and the deprotection step was repeated a second time.

The following three amino acids were sequentially coupled to the resin bound peptide using standard amidation conditions utilizing HCTU as the coupling agent: Fmoc-L-Leu-OH (6.96 g, 19.70 mmol, 1.2 eq)); Fmoc-N-Me-L-Lys(Boc)-OH (6.70 g, 13.88 mmol, 1.5 eq)); Fmoc-L-Cyclopropylglycine (2.29 g, 6.78 mmol, 1.2 eq). After the final amidation step, the resin-bound peptide with an Fmoc-protected N-terminus was washed with DMF, and the solution was drained.

### Transformation 7 to 9:

1% TFA in DCM (1 g resin/10 mL) was added to the peptidyl resin, and the mixture was stirred for 30 min and then filtered (repeated 6 times). The solution was neutralized to pH 7 with saturated sodium bicarbonate to afford the cleaved acyclic peptide. The brown solid was purified by flash column (ISCO 1000 g silica, 80% ethyl acetate in petroleum ether, gradient over 1.5 hr). The crude product was purified by Prep-TLC (Dichloromethane:Methanol=10/1, R_{f}=0.69) give 92 g to next step. Then 50% TFA in DCM (100 mL) was added to the acyclic peptide and stirred for 1 h at 15° C. LCMS showed reaction was completed. Concentrated in vacuum to give a residue, then the residue was neutralized to pH 7 with saturated sodium bicarbonate. The aqueous phase was extracted with DCM (300 mL*3). The combined organic phase was dried with anhydrous Na₂SO₄, filtered and concentrated in vacuum, which used directly for next step. Compound 9 (69 g, 72.45 mmol, 95.32% yield) was obtained as a pale brown solid.

### Preparation of Compound 10:

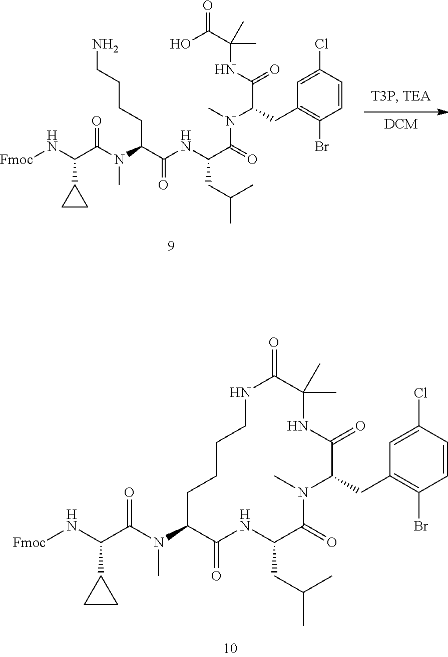

To a stirred solution of compound 9 (5 g, 5.25 mmol, 1 eq) in DCM (1800 mL) was added T₃P (6.68 g, 10.50 mmol, 6.24 mL, 50% purity, 2 eq) and TEA (2.66 g, 26.25 mmol, 3.65 mL, 5 eq) at 15° C., and the mixture was stirred for 1 h at 15° C. LCMS showed the compound 9 was consumed and main peak with desired MS was detected. The reaction mixture was concentrated in vacuum to about DCM (100 mL) and was added into water (100 mL). The organic phase was washed with water (100 mL×2). The combined aqueous phase was extracted with dichloromethane (100 mL×3). The combined organic phase was dried with anhydrous Na₂SO₄, filtered and concentrated in vacuum. TLC (ethyl acetate:methanol=20/1, R_{f}=0.57). The crude product was purified by flash column (ISCO 700 g silica, 80% ethyl acetate in petroleum ether, gradient over 100 min). Compound 10 (25.2 g, 19.96 mmol, 29.24% yield, 74% purity) was obtained as a pale brown solid. LCMS m/z 935.2 [M+H]⁺

### Preparation of Compound 11:

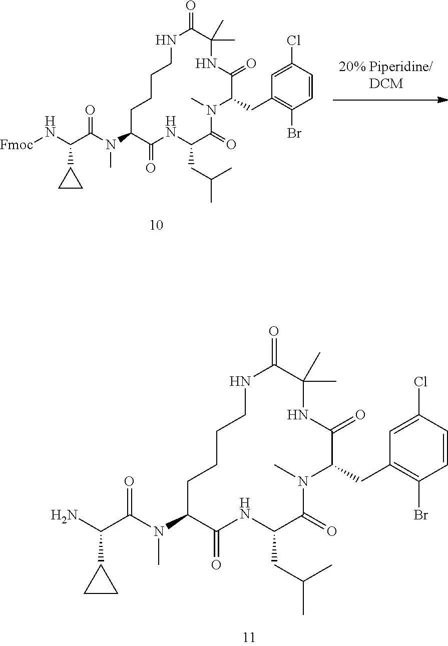

To a solution of compound 10 (22 g, 23.55 mmol, 1 eq) was added Piperidine (37.94 g, 445.54 mmol, 44 mL, 18.92 eq) (20% piperidine in DCM) at 15° C. The reaction was stirred at 15° C. for 1 h. TLC (ethyl acetate:methanol=1/1, R_{f}=0.22) showed compound 10 was consumed completely and one new spot formed. To the mixture was added saturated ammonium chloride solution (150 mL). The organic phase was washed with saturated ammonium chloride solution (150 mL*2). The combined aqueous phase was extracted with DCM (50 mL). The combined organic phase was dried with anhydrous Na₂SO₄, filtered and concentrated in vacuum. Compound 11 (21 g, crude) was obtained as a pale brown solid. LCMS m/z 713.2 [M+H]⁺

### Preparation of UX-0066:

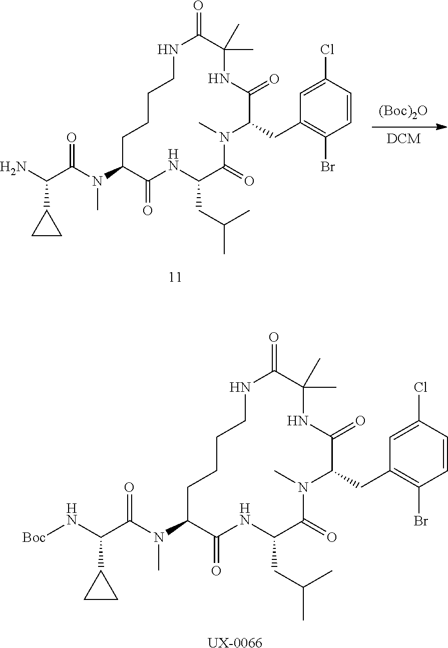

To a stirred solution of compound 11 (21 g, 29.49 mmol, 1 eq) in DCM (250 mL) was added TEA (14.92 g, 147.45 mmol, 20.52 mL, 5 eq), tert-butoxycarbonyl tert-butyl carbonate (32.18 g, 147.45 mmol, 33.87 mL, 5 eq) at 15° C. The reaction was stirred at 15° C. for 1 h. LCMS showed compound 11 was consumed completely and main peak with desired mass was detected. The solution was added saturated ammonium chloride solution (200 mL*3). The combined aqueous phase was extracted with DCM (100 mL). The combined organic phase was dried with anhydrous Na₂SO₄, filtered and concentrated in vacuum. The residue was purified by flash column (ISCO 600 g silica, 70-80% ethyl acetate in petroleum ether, gradient over 600 min). Compound UX-0066 (14.07 g, 17.24 mmol, 58.45% yield) was obtained as a pale yellow solid.

¹H NMR (400 MHz, METHANOL-d₄) δ 8.84-8.57 (m, 1H), 7.61 (d, J=8.6 Hz, 1H), 7.45-7.36 (m, 1H), 7.32 (br d, J=8.0 Hz, 1H), 7.25 (dd, J=2.4, 8.5 Hz, 1H), 7.16-7.09 (m, 1H), 6.73-6.59 (m, 1H), 5.49 (s, 1H), 5.05 (br d, J=10.6 Hz, 1H), 4.69-4.54 (m, 1H), 4.38 (br d, J=5.4 Hz, 1H), 4.30-4.18 (m, 1H), 3.77-3.60 (m, 1H), 3.51-3.33 (m, 2H), 3.14 (s, 2H), 3.01-2.95 (m, 1H), 2.94-2.86 (m, 1H), 2.82 (br d, J=4.4 Hz, 1H), 2.79 (s, 2H), 1.99-1.68 (m, 4H), 1.67-1.58 (m, 4H), 1.57-1.40 (m, 14H), 1.40-1.25 (m, 2H), 1.20-1.09 (m, 1H), 1.02-0.94 (m, 3H), 0.83 (br d, J=6.6 Hz, 3H), 0.58 (br d, J=7.8 Hz, 1H), 0.52-0.22 (m, 3H).

### 2. Solution Phase Synthesis—General Methods

xxiv. Suzuki—Suzuki Coupling Onto 2-Bromo, 5-chlorophenylalanine Method

The following example uses UX-0066 as a substrate. It is understood that non-reactive portions of the substrates can be modified and total reaction volume can be changed without departing from these teachings.

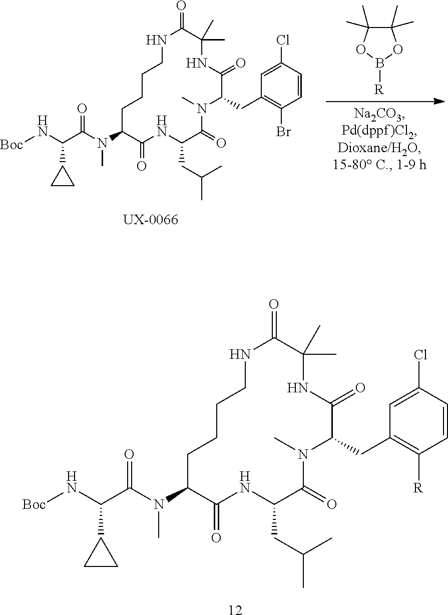

To a stirred solution of UX-0066 (3.5 g, 4.31 mmol, 1 eq) in dioxane (100 mL) and Water (10 mL) was added a pinacolborane (6.46 mmol, 1.5 eq), and Na₂CO₃ or K₂CO₃ (8.62 mmol, 2 eq) at 15° C. The mixture was degassed with nitrogen three times. Then Pd(dppf)Cl2 (315.30 mg, 430.91 umol, 0.1 eq) was added and the mixture was degassed with nitrogen for three times. The mixture was heated to 80° C. and stirred for 1-9 h under nitrogen atmosphere. The reaction mixture was cooled to room temperature. Then the mixture was added to water (100 mL) and ethyl acetate (100 mL). The aqueous phase was extracted with ethyl acetate (80 mL*3). The combined organic phases were dried with anhydrous Na₂SO₄, filtered and concentrated in vacuum to get a residue. The disappearance of starting material was confirmed by TLC. The residue was purified by flash column (ISCO 120 g silica, 5-10% dichloromethane in methanol, gradient over 20 min to give Compound 12 (2.7 g, 3.32 mmol, 77.03% yield).

xxv. SHATU—Solution Dipeptide Coupling Method

The following example uses Boc-protected macrocycle 12 as a substrate. It is understood that Boc protected substrates of different chemical compositions can be used as a starting point to complete the described reaction.

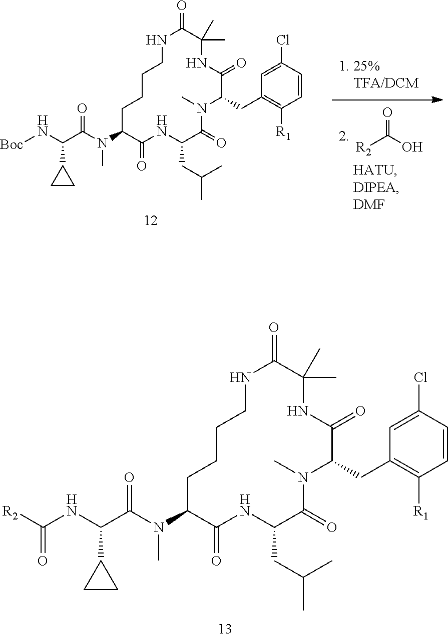

Boc-protected macrocycle 12 (˜50 mg) was dissolved in 25% TFA in DCM (5 mL). The reaction was monitored by LCMS for the disappearance of the starting material. Upon completion, the reaction was concentrated. The crude oil was co-evaporated with DCE (5 mL×2). The crude material was carried onto the subsequent step. Crude deprotected macrocycle (˜50 mg, ˜71 μmol), Dipeptide carboxylic acid (R₂—C(O)OH), 1.1 Eq, ˜79 μmol), and HATU (30 mg, 1.1 Eq, 79 μmol) were dissolved in DMF (2 mL). DIPEA (46 mg, 62 μL, 5 Eq, 0.36 mmol) was then added and the reaction was stirred until the completion of starting material by LCMS. Upon completion, the reaction was neutralized with 1N HCl and directly purified by RP-HPLC to give 13.

xxvi. SAc—Post-Cyclization Solution Acylation Method

The following example uses hydroxy-terminated macrocycle 14 as a substrate. It is understood that hydroxy-terminated substrates of different chemical compositions can be used as a starting point to complete the described reaction.

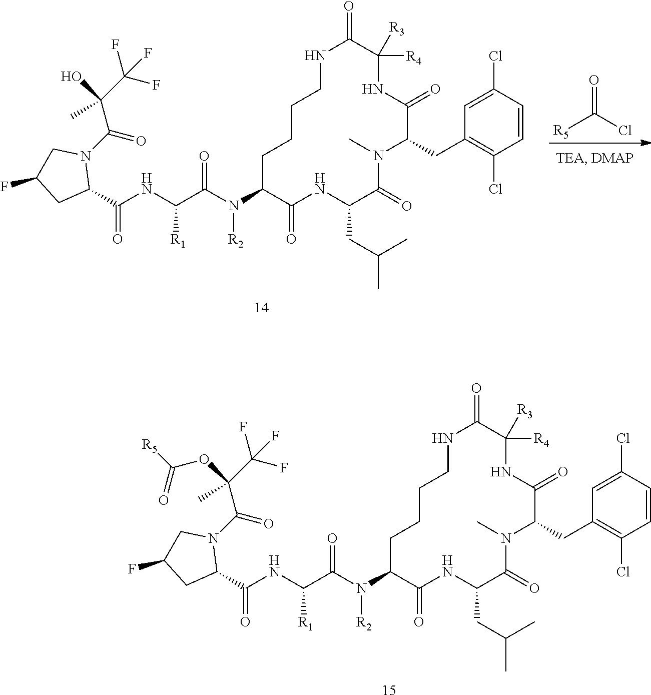

Hydroxy-terminated macrocycle 14 (˜50 mg, 1 Eq, 54 μmol) was dissolved in DCM (1 mL) and cooled in an ice bath. TEA (16 mg, 23 μL, 3 Eq, 0.16 mmol), DMAP (6.6 mg, 1 Eq, 54 μmol) and an acid chloride (4 Eq, 0.22 mmol) were added sequentially. Upon the consumption of starting material, the reaction was diluted with DCM (5 mL) and extracted with 1 N HCl (1 mL). The organic layer was dried, filtered, and concentrated. the crude mixture was purified by RP-HPLC to afford an acylated macrocycle 15 (46% yield) as a white solid.

### 3. Exemplary Compounds—Summary Tables

Table 5, below, lists the components and procedures used to prepared the listed exemplified compounds of Formula I. The macrocycle starting material used is a cyclic compound described in Table 2A and B. The Acyl Group components in Table 5 are listed using a short hand name that is identified in Table 1. Transformation 19 in Table 5 lists the conditions used to acylate the N-terminus of a macrocycle using abbreviations identified in the preceding general methods subheading.

Table 5 also includes the expected and observed mass for each exemplary compound.

Table 6A and B, below, lists the components and procedures used to prepared the listed exemplified compounds of Formula I. The exemplary compounds in these tables used UX-0066 as the starting macrocycle and include additional post cyclization in solution modifications. Procedures to prepare the UX-0066 starting material area described in a subheading above. The Boronic Acid and Dipeptide components in Table 6A are listed using a shorthand name that is identified in Table 1. The following generally describes the function of each listed step:
Transformation 20: Suzuki Coupling of Boronic Acid to Macrocycle Core
Transformation 21: Couples Dipeptide to N-terminus of the Macrocycle Core

Table 6B also includes the expected and observed mass for each exemplary compound.

TABLE 5
Components, Procedures, and Analytical Data for in solution
N-acylated Exemplay Compounds of the Present Disclosure

| | | | Transformation | Expected | Observed |
|---|---|---|---|---|---|
| Ex. | Macrocycle | Acyl Group | 19 | Mass | Mass |
| 681 | Ex. 210 | IsoBu | SAc | 992.93 | 992.4 |
| 682 | Ex. 455 | AcCl | SAc | 1004.94 | 1004.39 |

TABLE 6A
Components of Additional Exemplay Compounds of the Present Disclosure
Prepared with Post Cycliczation In Solution Modifications

| | Macrocycle | Boronic | |
|---|---|---|---|
| Ex. | Core | Acid | Dipeptide |
| 683 | UX-0066 | B0001 | Dip0018 |
| 684 | UX-0066 | B0002 | Dip0018 |
| 685 | UX-0066 | B0003 | Dip0018 |
| 686 | UX-0066 | | Dip0018 |
| 687 | UX-0066 | B0002 | Dip0023 |
| 688 | UX-0066 | B0003 | Dip0023 |
| 689 | UX-0066 | B0004 | Dip0018 |
| 690 | UX-0066 | B0007 | Dip0018 |
| 691 | UX-0066 | B0005 | Dip0018 |
| 692 | UX-0066 | B0006 | Dip0018 |
| 693 | UX-0066 | B0001 | Dip0019 |

TABLE 6B
Procedures, and Analytical Data of Additional Exemplary
Compounds of the Present Disclosure Prepared with Post
Cycliczation In Solution Modifications

| | Transformation | Transformation | Expected | Observed |
|---|---|---|---|---|
| Ex. | 20 | 21 | Mass | Mass |
| 683 | Suzuki | SHATU | 1057.57 | 1057.54 |
| 684 | Suzuki | SHATU | 1044.59 | 1044.51 |
| 685 | Suzuki | SHATU | 1030.55 | 1030.53 |
| 686 | | SHATU | 1013.32 | 1012.36 |
| 687 | Suzuki | SHATU | 994.59 | 994.52 |
| 688 | Suzuki | SHATU | 980.54 | 980.56 |
| 689 | Suzuki | SHATU | 1016.52 | 1016.55 |
| 690 | Suzuki | SHATU | 1028.54 | 1028.61 |
| 691 | Suzuki | SHATU | 1056.55 | 1056.53 |
| 692 | Suzuki | SHATU | 1041.53 | 1041.51 |
| 693 | Suzuki | SHATU | 1113.69 | 1113.64 |

Table 7, below, lists the full chemical structure for each exemplified compound in Table 5 and Table 6A/B.

TABLE 7
Chemical Structure for Exemplary Compounds of Table 5 and Table 6A/B.

| Ex. | Structure |
|---|---|
| 683 | 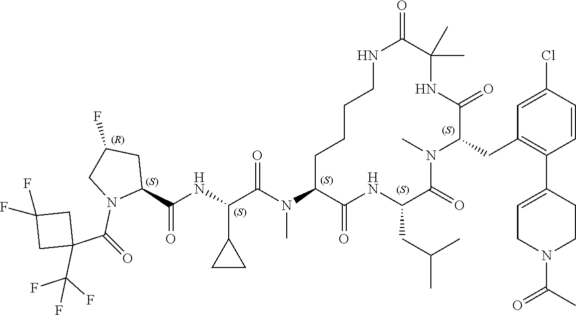 |
| 684 | 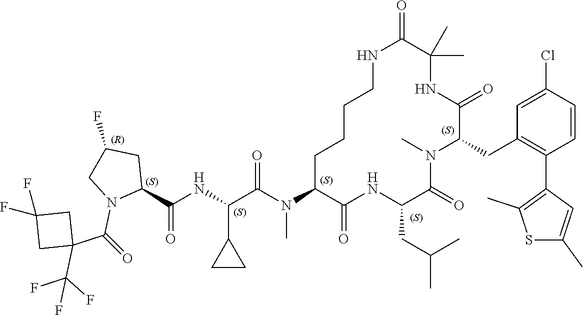 |
| 685 | 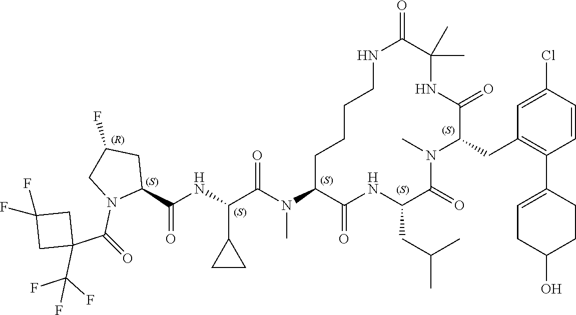 |
| 686 | 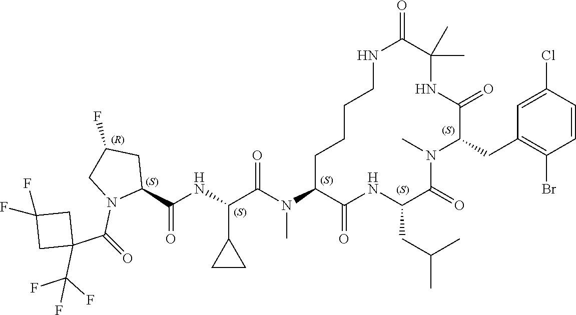 |
| 687 | 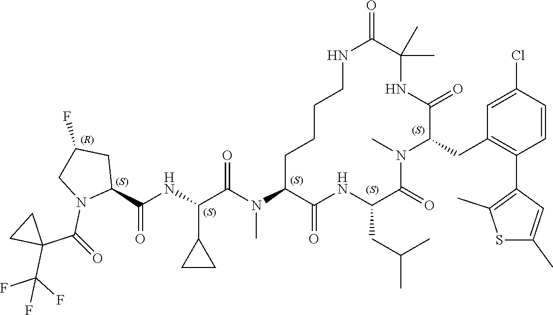 |
| 688 | 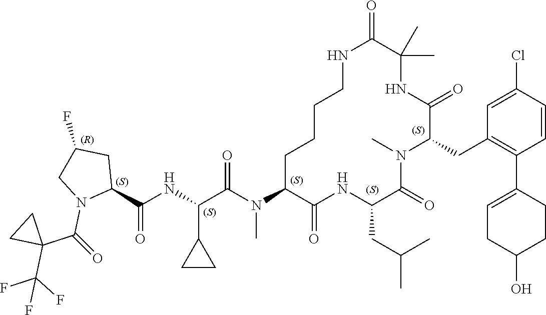 |
| 689 | 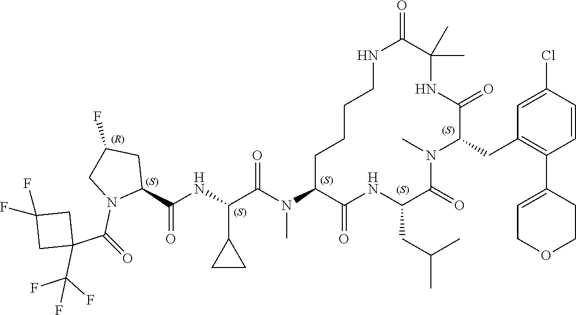 |
| 690 | 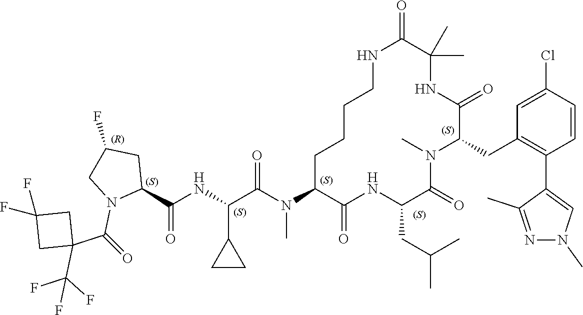 |
| 691 | 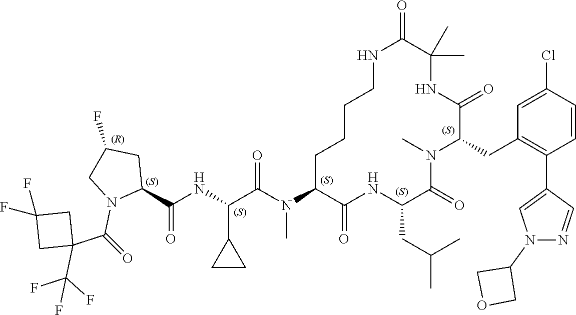 |
| 692 | 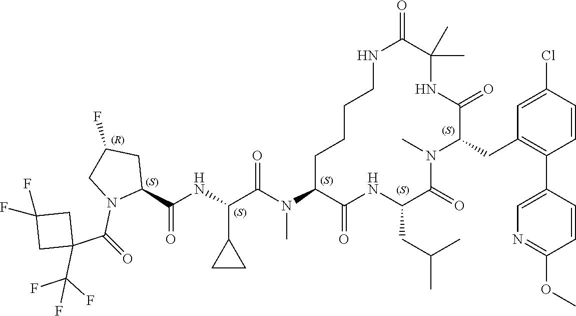 |
| 693 | 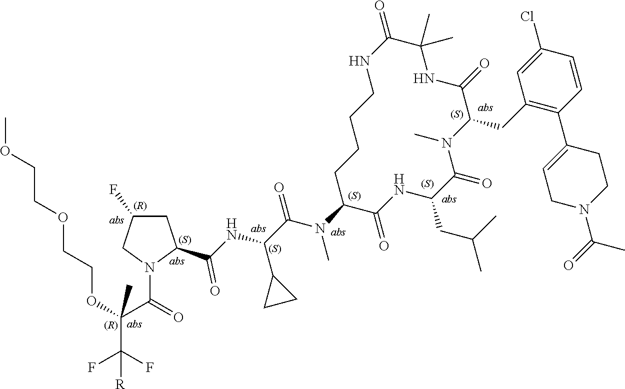 |

### D. Biological Examples

### 1. Fluorescence Polarization Assay

Binding affinity for the compounds of Formula I were determined by Fluorescence Polarization (FP) competitive assay based on previously established protocols (Andrews et. al., Org Biomol Chem. 2004. 2(19):2735-41; Premnath et. al., J Med Chem. 2015. 58(1):433-42) with modifications as described below. Cyclin/CDK protein complexes were sourced as follows: CyclinA2/CDK2 (CRELUX Protein Services), CyclinB1/CDK1 (Eurofins, discovery. Cat. No. 14-450) and CyclinEl/CDK2 (Eurofins, discovery. Cat. No. 14-475).

FP binding assays were performed in 25 mM HEPES pH 7.5, 100 mM NaCl, 1 mM DTT, 0.01% NP-40 and 1 mg/mL BSA for all 3 protein complexes in black 96-well plates. After experimental plates are set, they were equilibrated by gentle mixing by placing them on an orbital shaker at 100 rpm for 2 hours at room temperature and then read on a SpectraMax i3X Multi-Mode Microplate Detection platform.

Affinity of the Cyclin/Cdk complexed for the fluorescent labeled probe was determined by adding increasing concentration of each protein complex in buffer containing a carboxyfluorescein labeled probe (FAM probe) at 2 nM (preparation of FAM probe is described below). The half maximal concentration of protein needed for the maximal FP signal were 2 nM for Cyclin A2/Cdk2, 9 nM for Cyclin B1/Cdk1 and 3 nM for Cyclin E1/Cdk2. Methods to prepare the FAM probe are described in the heading below.

The protein concentration used for the competitive FP assays were 8 nM for Cyclin A2/Cdk2 and 10 nM for Cyclin B1/Cdk1 and Cyclin E1/Cdk2 with 2 nM of FAM probe FAM probe. Under these conditions, the dynamic range was about 120 mP 100% binding of FAM probe and complete inhibition of binding by excess of an unlabeled competitor compound, with all experiment showing a Z′ factor>0.80. IC₅₀ for test compounds were determined in eight point serial dilution dose response curves. Reported IC₅₀ are the average of 2-3 independent experiments. Data from these assays are reported in Table 8 and Table 9

TABLE 8
Cyclin A Activity Data of Exemplary Compounds

| | Cyclin A | | Cyclin A |
|---|---|---|---|
| | FP IC50 | | FP IC50 |
| Example | (UM) | Example | (uM) |
| 1 | 0.04 | 348 | 0.06 |
| 2 | 0.21 | 349 | 0.28 |
| 3 | 0.05 | 350 | 0.18 |
| 4 | 0.29 | 351 | 0.07 |
| 5 | 0.06 | 352 | 0.08 |
| 6 | 0.13 | 353 | 0.05 |
| 7 | 0.08 | 354 | 0.22 |
| 8 | 0.29 | 355 | 0.09 |
| 9 | 0.16 | 356 | 0.29 |
| 10 | 0.27 | 357 | 0.28 |
| 11 | 0.07 | 358 | 0.23 |
| 12 | 0.04 | 359 | 0.12 |
| 13 | 0.05 | 360 | 0.07 |
| 14 | 0.09 | 361 | 0.15 |
| 15 | 0.05 | 362 | 0.09 |
| 16 | 0.12 | 363 | 0.25 |
| 17 | 0.04 | 364 | 0.24 |
| 18 | 0.17 | 365 | 0.19 |
| 19 | 0.28 | 366 | 0.23 |
| 20 | 0.04 | 367 | 0.08 |
| 21 | 0.04 | 368 | 0.12 |
| 22 | 0.18 | 369 | 0.06 |
| 23 | 0.12 | 370 | 0.09 |
| 24 | 0.26 | 371 | 0.27 |
| 25 | 0.05 | 372 | 0.27 |
| 26 | 0.12 | 373 | 0.12 |
| 27 | 0.27 | 374 | 0.11 |
| 28 | 0.29 | 375 | 0.08 |
| 29 | 0.19 | 376 | 0.13 |
| 30 | 0.26 | 377 | 0.07 |
| 31 | 0.24 | 378 | 0.30 |
| 32 | 0.29 | 379 | 0.06 |
| 33 | 0.21 | 380 | 0.13 |
| 34 | 0.20 | 381 | 0.09 |
| 35 | 0.27 | 382 | 0.28 |
| 36 | 0.22 | 383 | 0.07 |
| 37 | 0.16 | 384 | 0.04 |
| 38 | 0.26 | 385 | 0.02 |
| 39 | 0.22 | 386 | 0.17 |
| 40 | 0.24 | 387 | 0.29 |
| 41 | 0.28 | 388 | 0.15 |
| 42 | 0.10 | 389 | 0.11 |
| 43 | 0.11 | 390 | 0.16 |
| 44 | 0.15 | 391 | 0.12 |
| 45 | 0.19 | 392 | 0.22 |
| 46 | 0.16 | 393 | 0.10 |
| 47 | 0.19 | 394 | 0.18 |
| 48 | 0.11 | 395 | 0.17 |
| 49 | 0.28 | 396 | 0.29 |
| 50 | 0.20 | 397 | 0.05 |
| 51 | 0.27 | 398 | 0.24 |
| 52 | 0.15 | 399 | 0.27 |
| 53 | 0.19 | 400 | 0.29 |
| 54 | 1.42 | 401 | 0.08 |
| 55 | 0.18 | 402 | 0.07 |
| 56 | 0.13 | 403 | 0.09 |
| 57 | 0.04 | 404 | 0.22 |
| 58 | 0.21 | 405 | 0.15 |
| 59 | 0.04 | 406 | 0.17 |
| 60 | 0.06 | 407 | 0.07 |
| 61 | 0.23 | 408 | 0.14 |
| 62 | 0.22 | 409 | 0.06 |
| 63 | 0.16 | 410 | 0.10 |
| 64 | 0.27 | 411 | 0.06 |
| 65 | 0.20 | 412 | 0.14 |
| 66 | 0.05 | 413 | 0.10 |
| 67 | 0.19 | 414 | 0.08 |
| 68 | 0.23 | 415 | 0.24 |
| 69 | 0.22 | 416 | 0.13 |
| 70 | 0.10 | 417 | 0.10 |
| 71 | 0.17 | 418 | 0.32 |
| 72 | 0.08 | 419 | 0.08 |
| 73 | 0.04 | 420 | 0.06 |
| 74 | 0.04 | 421 | 0.17 |
| 75 | 0.11 | 422 | 0.04 |
| 76 | 0.05 | 423 | 0.29 |
| 77 | 0.03 | 424 | 0.29 |
| 78 | 0.17 | 425 | 0.27 |
| 79 | 0.25 | 426 | 0.07 |
| 80 | 0.04 | 427 | 0.28 |
| 81 | 0.07 | 428 | 0.17 |
| 82 | 0.04 | 429 | 0.21 |
| 83 | 0.28 | 430 | 0.27 |
| 84 | 0.22 | 431 | 0.03 |
| 85 | 0.25 | 432 | 0.02 |
| 86 | 0.28 | 433 | 0.03 |
| 87 | 0.23 | 434 | 0.02 |
| 88 | 0.29 | 435 | 0.02 |
| 89 | 0.37 | 436 | 0.02 |
| 90 | 0.09 | 437 | 0.14 |
| 91 | 0.10 | 438 | 0.09 |
| 92 | 0.04 | 439 | 0.08 |
| 93 | 0.16 | 440 | 0.13 |
| 94 | 0.04 | 441 | 0.20 |
| 95 | 0.16 | 442 | 0.03 |
| 96 | 0.04 | 443 | 0.13 |
| 97 | 0.12 | 444 | 0.03 |
| 98 | 0.10 | 445 | 0.04 |
| 99 | 0.22 | 446 | 0.06 |
| 100 | 0.12 | 447 | 0.09 |
| 101 | 0.08 | 448 | 0.12 |
| 102 | 0.03 | 449 | 0.15 |
| 103 | 0.11 | 450 | 0.14 |
| 104 | 0.06 | 451 | 0.15 |
| 105 | 0.15 | 452 | 0.12 |
| 106 | 0.14 | 453 | 0.16 |
| 107 | 0.20 | 454 | 0.19 |
| 108 | 0.03 | 455 | 0.45 |
| 109 | 0.22 | 456 | 0.14 |
| 110 | 0.28 | 457 | 0.16 |
| 111 | 0.17 | 458 | 0.12 |
| 112 | 0.27 | 459 | 0.10 |
| 113 | 0.07 | 460 | 0.06 |
| 114 | 0.05 | 461 | 0.20 |
| 115 | 0.06 | 462 | 0.25 |
| 116 | 0.02 | 463 | 0.09 |
| 117 | 0.05 | 464 | 0.05 |
| 118 | 0.04 | 465 | 0.03 |
| 119 | 0.04 | 466 | 0.04 |
| 120 | 0.17 | 467 | 0.11 |
| 121 | 0.24 | 468 | 0.21 |
| 122 | 0.09 | 469 | 0.13 |
| 123 | 0.27 | 470 | 0.04 |
| 124 | 0.18 | 471 | 0.08 |
| 125 | 0.26 | 472 | 0.26 |
| 126 | 0.07 | 473 | 0.09 |
| 127 | 0.03 | 474 | 0.04 |
| 128 | 0.03 | 475 | 0.07 |
| 129 | 0.02 | 476 | 0.03 |
| 130 | 0.10 | 477 | 0.14 |
| 131 | 0.21 | 478 | 0.10 |
| 132 | 0.28 | 479 | 0.08 |
| 133 | 0.03 | 480 | 0.06 |
| 134 | 0.19 | 481 | 0.12 |
| 135 | 0.30 | 482 | 0.09 |
| 136 | 0.24 | 483 | 0.03 |
| 137 | 0.04 | 484 | 0.11 |
| 138 | 0.08 | 485 | 0.07 |
| 139 | 0.22 | 486 | 0.09 |
| 140 | 0.24 | 487 | 0.23 |
| 141 | 0.16 | 488 | 0.07 |
| 142 | 0.11 | 489 | 0.24 |
| 143 | 0.09 | 490 | 0.07 |
| 144 | 0.10 | 491 | 0.09 |
| 145 | 0.21 | 492 | 0.25 |
| 146 | 0.11 | 493 | 0.10 |
| 147 | 0.28 | 494 | 0.19 |
| 148 | 0.74 | 495 | 0.29 |
| 149 | 0.08 | 496 | 0.09 |
| 150 | 0.10 | 497 | 0.09 |
| 151 | 0.20 | 498 | 0.19 |
| 152 | 0.27 | 499 | 0.12 |
| 153 | 0.04 | 500 | 0.05 |
| 154 | 0.04 | 501 | 0.16 |
| 155 | 0.13 | 502 | 0.12 |
| 156 | 0.10 | 503 | 0.06 |
| 157 | 0.11 | 504 | 0.10 |
| 158 | 0.23 | 505 | 0.05 |
| 159 | 0.25 | 506 | 0.16 |
| 160 | 0.08 | 507 | 0.06 |
| 161 | 0.12 | 508 | 0.12 |
| 162 | 0.08 | 509 | 0.15 |
| 163 | 0.14 | 510 | 0.24 |
| 164 | 0.09 | 511 | 0.21 |
| 165 | 0.13 | 512 | 0.09 |
| 166 | 0.04 | 513 | 0.08 |
| 167 | 0.22 | 514 | 0.27 |
| 168 | 0.19 | 515 | 0.08 |
| 169 | 0.15 | 516 | 0.03 |
| 170 | 0.16 | 517 | 0.10 |
| 171 | 0.04 | 518 | 0.30 |
| 172 | 0.04 | 519 | 0.04 |
| 173 | 0.20 | 520 | 0.02 |
| 174 | 0.10 | 521 | 0.03 |
| 175 | 0.22 | 522 | 0.05 |
| 176 | 0.25 | 523 | 0.06 |
| 177 | 0.14 | 524 | 0.22 |
| 178 | 0.11 | 525 | 0.08 |
| 179 | 0.26 | 526 | 0.12 |
| 180 | 0.09 | 527 | 0.03 |
| 181 | 0.09 | 528 | 0.04 |
| 182 | 0.04 | 529 | 0.03 |
| 183 | 0.14 | 530 | 0.05 |
| 184 | 0.26 | 531 | 0.04 |
| 185 | 0.16 | 532 | 0.20 |
| 186 | 0.26 | 533 | 0.11 |
| 187 | 0.07 | 534 | 0.27 |
| 188 | 0.15 | 535 | 0.06 |
| 189 | 1.01 | 536 | 0.26 |
| 190 | 0.25 | 537 | 0.05 |
| 191 | 0.30 | 538 | 0.13 |
| 192 | 0.25 | 539 | 0.23 |
| 193 | 0.07 | 540 | 0.11 |
| 194 | 0.12 | 541 | 0.11 |
| 195 | 0.14 | 542 | 0.13 |
| 196 | 0.13 | 543 | 0.19 |
| 197 | 0.13 | 544 | 0.09 |
| 198 | 0.19 | 545 | 0.15 |
| 199 | 0.02 | 546 | 0.65 |
| 200 | 0.17 | 547 | 1.39 |
| 201 | 0.17 | 548 | 1.13 |
| 202 | 0.16 | 549 | 0.09 |
| 203 | 0.26 | 550 | 0.12 |
| 204 | 0.27 | 551 | 0.30 |
| 205 | 0.28 | 552 | 0.20 |
| 206 | 0.06 | 553 | 0.22 |
| 207 | 0.25 | 554 | 0.06 |
| 208 | 0.13 | 555 | 0.11 |
| 209 | 0.05 | 556 | 0.07 |
| 210 | 0.06 | 557 | 0.25 |
| 211 | 0.23 | 558 | 0.20 |
| 212 | 0.29 | 559 | 0.16 |
| 213 | 0.19 | 560 | 0.03 |
| 214 | 0.26 | 561 | 0.25 |
| 215 | 0.14 | 562 | 0.09 |
| 216 | 0.28 | 563 | 0.32 |
| 217 | 0.27 | 564 | 0.19 |
| 218 | 0.28 | 565 | 0.14 |
| 219 | 0.14 | 566 | 0.23 |
| 220 | 0.21 | 567 | 0.26 |
| 221 | 0.22 | 568 | 0.03 |
| 222 | 0.17 | 569 | 0.08 |
| 223 | 0.14 | 570 | 0.06 |
| 224 | 0.13 | 571 | 0.13 |
| 225 | 0.03 | 572 | 0.04 |
| 226 | 0.10 | 573 | 0.20 |
| 227 | 0.19 | 574 | 0.24 |
| 228 | 0.08 | 575 | 0.10 |
| 229 | 0.18 | 576 | 0.21 |
| 230 | 0.25 | 577 | 0.18 |
| 231 | 0.08 | 578 | 0.02 |
| 232 | 0.15 | 579 | 0.07 |
| 233 | 0.29 | 580 | 0.14 |
| 234 | 0.22 | 581 | 0.28 |
| 235 | 0.21 | 582 | 0.04 |
| 236 | 0.08 | 583 | 0.05 |
| 237 | 0.14 | 584 | 0.29 |
| 238 | 0.14 | 585 | 0.04 |
| 239 | 0.09 | 586 | 0.14 |
| 240 | 0.06 | 587 | 0.09 |
| 241 | 0.21 | 588 | 0.13 |
| 242 | 0.24 | 589 | 0.05 |
| 243 | 0.15 | 590 | 0.03 |
| 244 | 0.28 | 591 | 0.08 |
| 245 | 0.19 | 592 | 0.13 |
| 246 | 0.25 | 593 | 0.08 |
| 247 | 0.20 | 594 | 0.03 |
| 248 | 0.08 | 595 | 0.09 |
| 249 | 0.21 | 596 | 0.03 |
| 250 | 0.09 | 597 | 0.20 |
| 251 | 0.06 | 598 | 0.10 |
| 252 | 0.24 | 599 | 0.02 |
| 253 | 0.16 | 600 | 0.02 |
| 254 | 0.17 | 601 | 0.04 |
| 255 | 0.25 | 602 | 0.15 |
| 256 | 0.09 | 603 | 0.18 |
| 257 | 0.02 | 604 | 0.23 |
| 258 | 0.23 | 605 | 0.04 |
| 259 | 0.29 | 606 | 0.13 |
| 260 | 0.18 | 607 | 0.05 |
| 261 | 0.21 | 608 | 0.06 |
| 262 | 0.12 | 609 | 0.04 |
| 263 | 0.15 | 610 | 0.14 |
| 264 | 0.14 | 611 | 0.03 |
| 265 | 0.23 | 612 | 0.05 |
| 266 | 0.04 | 613 | 0.14 |
| 267 | 0.12 | 614 | 0.03 |
| 268 | 0.19 | 615 | 0.06 |
| 269 | 0.28 | 616 | 0.02 |
| 270 | 0.26 | 617 | 0.13 |
| 271 | 0.20 | 618 | 0.02 |
| 272 | 0.11 | 619 | 0.03 |
| 273 | 0.27 | 620 | 0.06 |
| 274 | 0.25 | 62 | 0.05 |
| 275 | 0.15 | 622 | 0.13 |
| 276 | 0.17 | 623 | 0.02 |
| 277 | 0.27 | 624 | 0.05 |
| 278 | 0.20 | 625 | 0.21 |
| 279 | 0.23 | 626 | 0.07 |
| 280 | 0.29 | 627 | 0.10 |
| 281 | 0.13 | 628 | 0.03 |
| 282 | 0.27 | 629 | 0.06 |
| 283 | 0.26 | 630 | 0.02 |
| 284 | 0.29 | 631 | 0.02 |
| 285 | 0.03 | 632 | 0.02 |
| 286 | 0.20 | 633 | 0.30 |
| 287 | 0.12 | 634 | 0.08 |
| 288 | 0.05 | 635 | 0.10 |
| 289 | 0.16 | 636 | 0.07 |
| 290 | 0.25 | 637 | 0.03 |
| 291 | 0.06 | 638 | 0.02 |
| 292 | 0.20 | 639 | 0.08 |
| 293 | 0.04 | 640 | 0.07 |
| 294 | 0.16 | 641 | 0.10 |
| 295 | 0.12 | 642 | 0.18 |
| 296 | 0.12 | 643 | 0.08 |
| 297 | 0.14 | 64 | 0.11 |
| 298 | 0.11 | 645 | 0.15 |
| 299 | 0.17 | 646 | 0.29 |
| 300 | 0.09 | 647 | 0.13 |
| 301 | 0.12 | 648 | 0.15 |
| 302 | 0.20 | 649 | 0.09 |
| 303 | 0.02 | 650 | 0.19 |
| 304 | 0.05 | 651 | 0.08 |
| 305 | 0.09 | 652 | 0.09 |
| 306 | 0.08 | 653 | 0.09 |
| 307 | 0.14 | 654 | 0.14 |
| 308 | 0.24 | 655 | 0.13 |
| 309 | 0.09 | 656 | 0.24 |
| 310 | 0.10 | 657 | 0.13 |
| 311 | 0.20 | 658 | 0.16 |
| 312 | 0.17 | 659 | 0.19 |
| 313 | 0.22 | 660 | 0.15 |
| 314 | 0.11 | 661 | 0.10 |
| 315 | 0.27 | 662 | 0.02 |
| 316 | 0.06 | 663 | 0.06 |
| 317 | 0.08 | 664 | 0.06 |
| 318 | 0.23 | 665 | 0.07 |
| 319 | 0.16 | 666 | 0.06 |
| 320 | 0.06 | 667 | 0.11 |
| 321 | 0.06 | 668 | 0.23 |
| 322 | 0.09 | 669 | 0.02 |
| 323 | 0.07 | 670 | 0.13 |
| 324 | 0.13 | 671 | 0.04 |
| 325 | 0.29 | 672 | 0.11 |
| 326 | 0.08 | 673 | 0.12 |
| 327 | 0.25 | 674 | 0.19 |
| 328 | 0.24 | 675 | 0.15 |
| 329 | 0.22 | 676 | 0.02 |
| 330 | 0.03 | 677 | 0.02 |
| 331 | 0.05 | 678 | 0.03 |
| 332 | 0.06 | 679 | 0.02 |
| 333 | 0.08 | 680 | >20 |
| 334 | 0.08 | 681 | 0.05 |
| 335 | 0.04 | 682 | 0.21 |
| 336 | 0.09 | 683 | 0.02 |
| 337 | 0.04 | 684 | 0.05 |
| 338 | 0.08 | 685 | 0.02 |
| 339 | 0.13 | 686 | 0.04 |
| 340 | 0.16 | 687 | 0.10 |
| 341 | 0.28 | 688 | 0.03 |
| 342 | 0.10 | 689 | 0.02 |
| 343 | 0.02 | 690 | 0.02 |
| 344 | 0.03 | 691 | 0.14 |
| 345 | 0.12 | 692 | 0.02 |
| 346 | 0.12 | 693 | 0.02 |
| 347 | 0.25 | | |

TABLE 9
Cyclin B and E Activity Data of Exemplary Compounds

| | Cyclin B | Cyclin E |
|---|---|---|
| | FP IC50 | FP IC50 |
| Example | (UM) | (UM) |
| 206 | 0.02 | 0.18 |
| 288 | 0.02 | 0.20 |
| 331 | 0.02 | 0.24 |
| 333 | 0.02 | 0.20 |
| 348 | 0.02 | 0.15 |
| 438 | 0.02 | 0.10 |

### Preparation of Fluorescent Probe (FAM Probe)

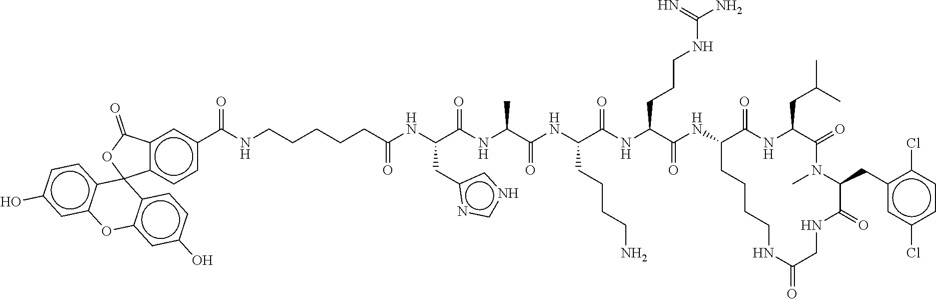

The fluorescent probe was synthesized via solid phase peptide synthesis followed by cyclization, fluorescent labeling, and deprotection in solution.

To load Fmoc-Glycine onto ˜50 mg of CTC resin, Fmoc-Glycine (G), CAS #29022-11-5, (4 equiv.) was dissolved in 1.0 mL of anhydrous NMP. Neat DIEA (8 equiv.) was added to the Fmoc-amino acid solution. The solution was dispensed in a peptide reactor vessel containing 50 mg of 2-chlorotrityl chloride (CTC) resin and was agitated for 2 hours at room temperature. The amino acid solution was drained then the resin was washed with 1.0 mL DMF three times. Unreacted CTC resin was capped with 1.0 mL solution of methanol:DMF (50:50), and DIEA (8 equiv.) for 10 minutes at room temperature. The methanol solution was drained then the resin was washed with 1.0 mL DMF three times. To remove Fmoc, A mixture of piperidine:DMF (20:80, 1 mL) was added to the resin and agitated for 10 to 15 minutes at room temperature. The piperidine solution was drained and then the resin was washed with 1.0 mL DMF three times.

A solution of Fmoc-L-2,5-dichlorophenylalanine-OH (25ClF), CAS #1260614-80-9, (4 equiv.), HATU (4 equiv.), and DIEA (8 equiv.) in 1.0 mL of anhydrous NMP was prepared. The mixture was allowed to pre-activate at room temperature for 5 minutes, and then was added to the resin and agitated at 35° C. for 30 minutes. The mixture was drained then the resin was washed with 1.0 mL of DMF three times. To remove Fmoc, A mixture of piperidine:DMF (20:80, 1 mL) was added to the resin and agitated for 10 to 15 minutes at room temperature. The piperidine solution was drained and then the resin was washed with 1.0 mL DMF three times.

To N-Methylate the amine of 25ClF, 2,6-lutidine (6 equiv.) dissolved in 0.5 mL of anhydrous DCE was added to the resin. 2-nitrobenzenesulfonyl chloride (5 equiv.) dissolved in 0.5 mL anhydrous toluene was added to the resin and then was agitated at 40 to 45° C. for 10 to 15 minutes. The mixture was drained, then the resin was washed with 1.0 mL of anhydrous toluene three times. The method was repeated twice. Triphenylphosphine (10 equiv.) dissolved in 0.7 mL anhydrous toluene was added to the resin. Dry methanol (MeOH), (20 equiv.) was added to the resin. Azodicarboxylate (10 equiv) was added to the resin and the mixture was agitated at 45° C. for 30 minutes. The mixture was drained and the resin was washed with 1.0 mL of anhydrous DMF three times. Alkylation was repeated twice. The nosyl group was then deprotected. A solution of 2-mercaptoethanol (5 equiv.) and 1,8-Diazabicyclo[5.4.0]undec-7-ene (5 equiv.) in 1.0 mL NMP was added to the resin and the mixture was agitated at 45° C. for 10 minutes. The mixture was drained and then the resin was washed with 1.0 mL of anhydrous DMF three times. Deprotection was repeated twice.

Fmoc-L-Leucine-OH (L), CAS #35661-60-0 (12 equiv.), HATU (4 equiv.), and DIEA (8 equiv.) in 1.0 mL of anhydrous NMP was prepared. The mixture was allowed to pre-activate at room temperature for 5 minutes, and then was added to the resin and agitated at 35° C. for 30 minutes. The mixture was drained then the resin was washed with 1.0 mL of DMF three times. To remove Fmoc, A mixture of piperidine:DMF (20:80, 1 mL) was added to the resin and agitated for 15 minutes at room temperature. The piperidine solution was drained and then the resin was washed with 1.0 mL DMF three times.

Fmoc-L-Lysine(Mtt)-OH (KMtt), CAS #167393-62-6, (4 equiv.), HATU (4 equiv.), and DIEA (8 equiv.) in 1.0 mL of anhydrous NMP was prepared. The mixture was allowed to pre-activate at room temperature for 5 minutes, and then was added to the resin and agitated at 35° C. for 30 minutes. The mixture was drained then the resin was washed with 1.0 mL of DMF three times. To remove Fmoc, A mixture of piperidine:DMF (20:80, 1 mL) was added to the resin and agitated for 15 minutes at room temperature. The piperidine solution was drained and then the resin was washed with 1.0 mL DMF three times.

Fmoc-L-Arginine(Pbf)-OH (RPbf), CAS #154445-77-9, (4 equiv.), HATU (4 equiv.), and DIEA (8 equiv.) in 1.0 mL of anhydrous NMP was prepared. The mixture was allowed to pre-activate at room temperature for 5 minutes, and then was added to the resin and agitated at 35° C. for 30 minutes. The mixture was drained then the resin was washed with 1.0 mL of DMF three times. To remove Fmoc, A mixture of piperidine:DMF (20:80, 1 mL) was added to the resin and agitated for 15 minutes at room temperature. The piperidine solution was drained and then the resin was washed with 1.0 mL DMF three times.

Fmoc-L-Lysine(Boc)-OH (KBoc), CAS #71989-26-9, (4 equiv.), HATU (4 equiv.), and DIEA (8 equiv.) in 1.0 mL of anhydrous NMP was prepared. The mixture was allowed to pre-activate at room temperature for 5 minutes, and then was added to the resin and agitated at 35° C. for 30 minutes. The mixture was drained then the resin was washed with 1.0 mL of DMF three times. To remove Fmoc, A mixture of piperidine:DMF (20:80, 1 mL) was added to the resin and agitated for 15 minutes at room temperature. The piperidine solution was drained and then the resin was washed with 1.0 mL DMF three times.

Fmoc-L-Alanine-OH (A), CAS #35661-39-3, (4 equiv.), HATU (4 equiv.), and DIEA (8 equiv.) in 1.0 mL of anhydrous NMP was prepared. The mixture was allowed to pre-activate at room temperature for 5 minutes, and then was added to the resin and agitated at 35° C. for 30 minutes. The mixture was drained then the resin was washed with 1.0 mL of DMF three times. To remove Fmoc, A mixture of piperidine:DMF (20:80, 1 mL) was added to the resin and agitated for 15 minutes at room temperature. The piperidine solution was drained and then the resin was washed with 1.0 mL DMF three times.

Fmoc-L-Histidine(Trt)-OH (HTrt), CAS #109425-51-6, (4 equiv.), HATU (4 equiv.), and DIEA (8 equiv.) in 1.0 mL of anhydrous NMP was prepared. The mixture was allowed to pre-activate at room temperature for 5 minutes, and then was added to the resin and agitated at 35° C. for 30 minutes. The mixture was drained then the resin was washed with 1.0 mL of DMF three times. To remove Fmoc, A mixture of piperidine:DMF (20:80, 1 mL) was added to the resin and agitated for 15 minutes at room temperature. The piperidine solution was drained and then the resin was washed with 1.0 mL DMF three times.

Fmoc-6-aminohexanoic acid (Ahx), CAS #88574-06-5, (4 equiv.), HATU (4 equiv.), and DIEA (8 equiv) in 1.0 mL of anhydrous NMP was prepared. The mixture was allowed to pre-activate at room temperature for 5 minutes, and then was added to the resin and agitated at 35° C. for 30 minutes. The mixture was drained and then the resin was washed with 1.0 mL of DMF three times.

To cleave peptide from CTC resin and simultaneously deprotect the Mtt protecting group, approximately 2 mL of a solution of 24% HFIP, 2% TIPS, in DCM was added to the polystyrene resin in a solid phase reaction vessel. The contents were shaken for 1 hour. The cleavage solution was filtered into a 50 mL conical vial. The cleaved resin was washed with an additional 2 mL of DCM and the wash was collected in the conical vial. The solution was evaporated in a Genevac. The linear peptide was purified via reverse-phase HPLC using an Acetonitrile/Water gradient with 0.05% formic acid and the purified fractions were pooled and lyophilized to yield white powder of intermediate X (M/z observed=1968.65 [M+H]⁺).

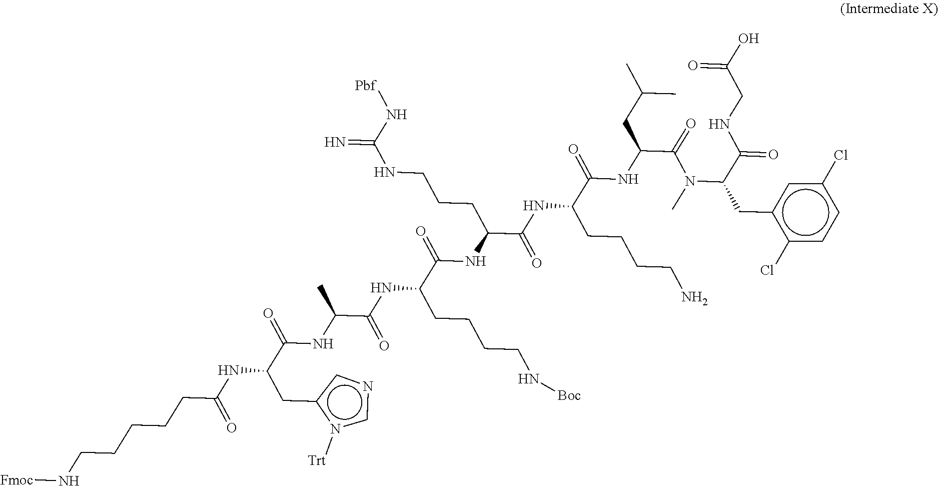

The linear intermediate X (˜15 mg) was cyclized using a medium volume, T3P solution cyclization method. The deprotected and purified linear product was transferred to a 50 mL conical vial and dissolved in 1 mL NMP followed by the addition of DIEA (0.5 mL) and DCM (35 mL). T3P (3 eqv) was added to the solution and the reaction pH was adjusted to pH 9 via dropwise addition of DIEA. The closed conical vial was agitated at room temperature for 2 hours at 150 rotations per minute. The solution was concentrated at 45° C. under reduced pressure in a Genevac system. The Fmoc group was then removed with the addition of a 10% of KOH/Water solution (5 mL) heated at 70° C. for 30 minutes. The resulting LCMS trace revealed that the trityl group had been unexpectedly removed during the cyclization and Fmoc-deprotection steps. The cyclic peptide was then purified via reverse phase HPLC using an Acetonitrile/Water gradient with 0.05% formic acid. The purified fractions were pooled and lyophilized to yield intermediate Y (M/z observed=1485.94 [M+Z]⁺).

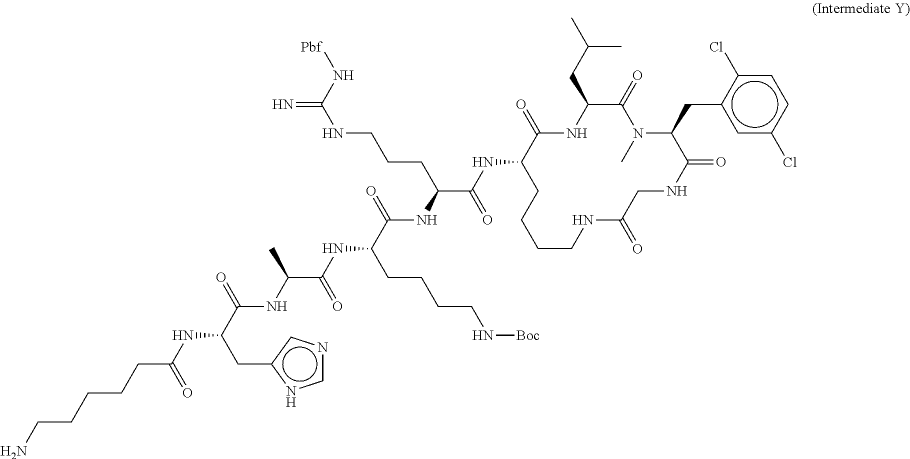

The probe was fluorescently labeled via a peptide coupling in solution. A solution of 5-carboxyfluorescein (CAS #76823-03-5, FAM) (4 equiv.), EDC (4 equiv.), HOAt (3.9 equiv.) and DIEA (8 equiv.) in 1.0 mL of anhydrous DCM was prepared. The mixture was allowed to pre-activate at room temperature for 5 minutes. Intermediate Y was added to the coupling solution, and the reaction was agitated at room temperature until starting material was not observed by LCMS, resulting in the formation of Intermediate Z (M/z observed=1844.29 [M+Z]⁺).

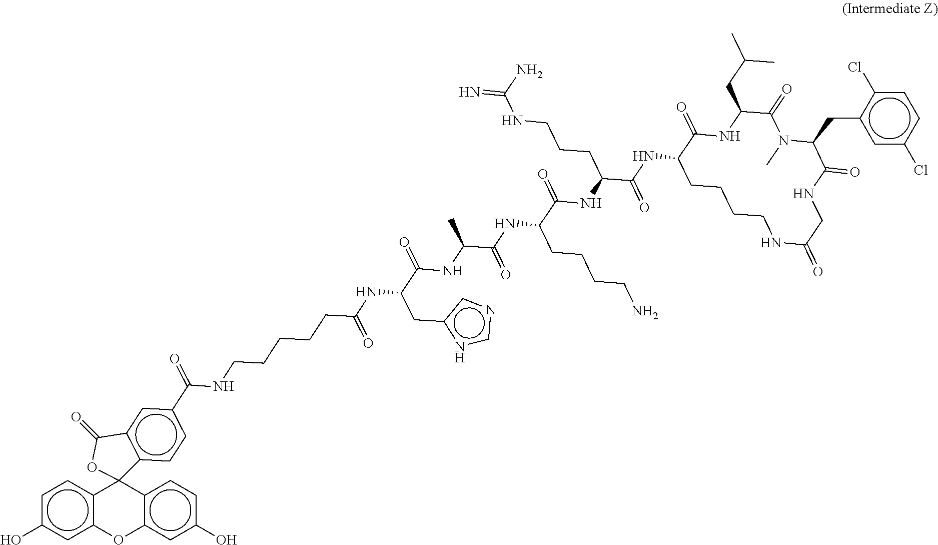

The Boc and Pbf protecting groups were removed from the cyclic intermediate Z by dissolving the cyclic peptide in a 1 mL solution of 90% TFA, 5% TIPS, 5% DCM and agitating for 1 hour. The reaction was monitored by LCMS for the disappearance of starting material. Upon completion, the reaction was concentrated. The crude material was co-evaporated with DCE (5 mL×2), and then purified via reverse phase-HPLC to yield fluorescent probe (FAM probe) (M/z observed=1492.14 [M+Z]⁺, 0.7 mg, 99% purity by HPLC).

### 2. Antiproliferative Activity Small Cell Lung Cancer Cell Lines

The following example describes the antiproliferative activity of an exemplary compound described herein (Example 456) in two Small Cell Lung Cancer (SCLC) cell lines (NCI-H69 and NCI-H1048). Both of these cell lines have defects in p53 gene/signaling and the Retinoblastoma gene (Rb) pathway, which drive aberrant activation of the Cyclin-E/Cdk2 complex, rapid progression through the G1-phase and defects in the transition checkpoint from G1 into the S-phase were where Cyclin-A/Cdk2 is activated to orchestrate DNA replication. Cyclin-A/Cdk2 and the sequential activation of Cyclin-A/Cdk1 and Cyclin-B/Cdk1 play critical roles in the transition from S-phase into G2-phase and into mitosis.

Example 456 has significant activity in five-day proliferation assays (˜3-4 cell number doublings), resulting in Growth Inhibition by 50% (GI₅₀) at concentration of 14 and 6 nM in NCI-H1048 and NCI-H69 cells, respectively. In contrast, Example 456 shows GI₅₀ of 14 μM in the human normal fibroblast cell line WI-38. Thus, Example 456 shows a 1000-fold growth inhibition selectivity for these two cancer cells cell lines as compared to a normal fibroblast cell line.

### 3. Target Engagement in Cells

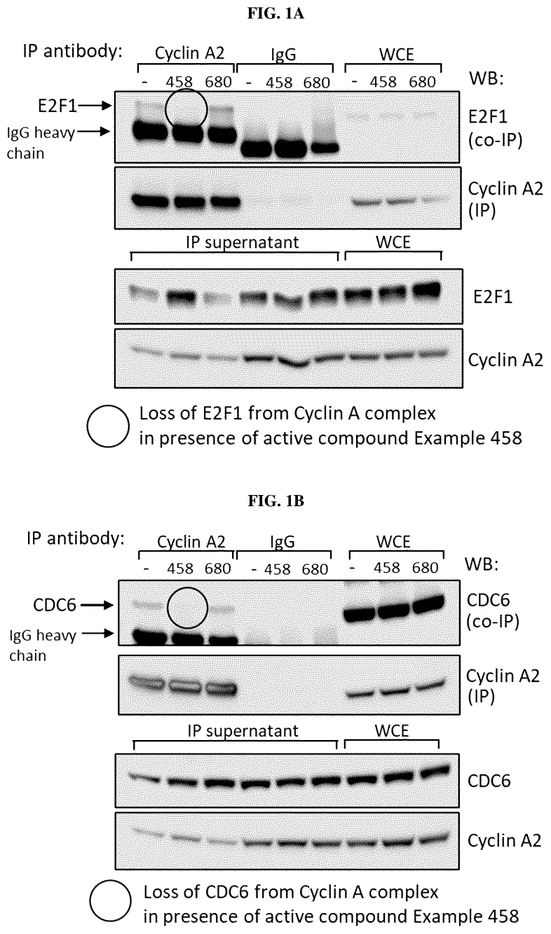

In addition to the foregoing data demonstrating biochemical target engagement with RxL domains on cyclins A, E and B, and inhibition of proliferation of SCLC cell lines, example compounds were evaluated for target engagement with cyclin A in cells using co-immunoprecipitation (see FIGS. 1A and 1B). Briefly, the SCLC cell line NCI-H1048 was grown in the presence of 300 nM of Example 458, its enantiomer Example 680 or no additive for 2 hours, lysed and the lysate immunoprecipitated with antibodies against Cyclin A2. The presence of CDK2 in the immunoprecipitates were confirmed by western blotting and the kinase activity of the Cyclin A2:CDK2 complex was demonstrated in a kinase activity assay (data not shown). Western blotting of the immunoprecipitates to detect the Cyclin A2:CDK2 substrates E2F1 or CDC6 that bind to Cyclin A2 via their RxL motifs demonstrated the presence of these substrates in the complexes incubated with the enantiomer Example 680 or no additive, but that incubation with Example 458 displaced both substrates from the complex (see FIGS. 1A and 1B), confirming the engagement of Example 458 with the Cyclin A target and resultant displacement of substrates from the CyclinA2:CDK2 complex in cells.

### 4. In Vivo Efficacy in Mouse Small Cell Lung Cancer Tumor Model Using the NCI-H69 Cancer Cell Line

To confirm that the observed biochemical and cellular activities can result in anti-tumor activity in an in vivo setting, Example 456 was used in a relevant mouse tumor model. In this study, vehicle negative control and paclitaxel positive control groups were included for comparison. In In this model mice were inoculated with 5×10⁶ NCI-H69 cells. Animals were randomized by tumor volume and IV drug treatment via the lateral tail vein was initiated when tumors reached 88-200 mm³. Example 456 was dosed at 50 and 100 mg/kg QD beginning on day 0 and continuing through day 13, and Paclitaxel was dosed at 20 mg/kg QOD for five doses beginning on day 0 (n=10 for all groups).

IV: intravenous; QD: once daily; QOD: once every two days; Q3D: once every three days; SEM: standard error of mean.

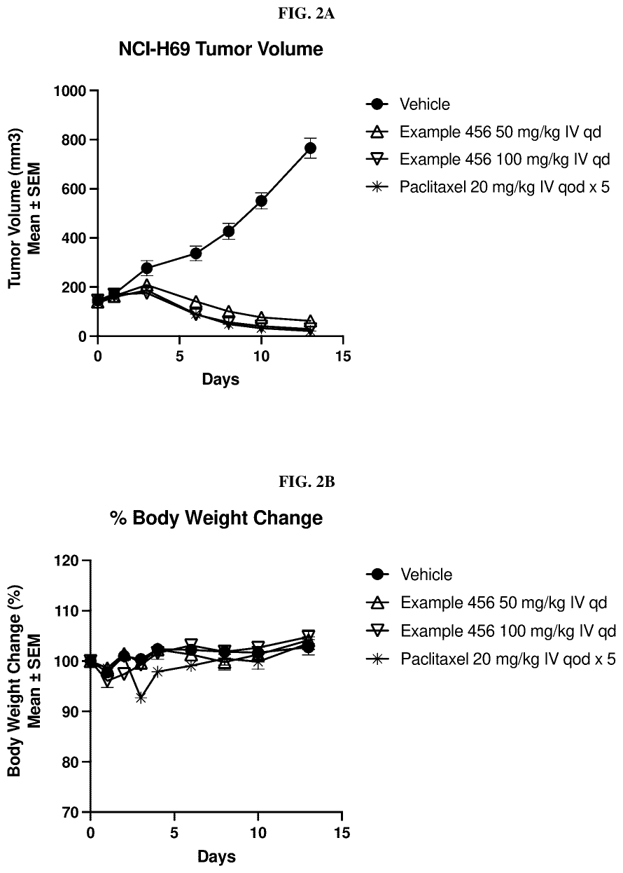

The results from this study, shown in FIGS. 2A and 2B, below, show the cyclin inhibitor Example 456 demonstrated strong anti-tumor activity.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound of Formula I 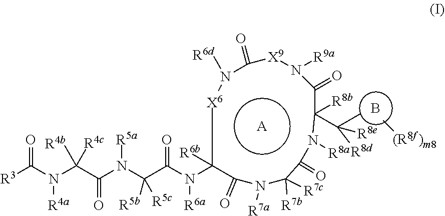
wherein
R³ is
(a) C₁₋₈ alkyl, C₂₋₈ alkenyl, C₂₋₈ alkynyl, or C₁₋₈ haloalkyl, each substituted with 0 to 5 R^{3a};
(b) C₃₋₁₂ cycloalkyl substituted with 0 to 5 R^{3b}; or
(c) heterocycloalkyl having 3 to 6 ring members and 1 to 3 heteroatoms each independently N, O, or S, wherein the heterocycloalkyl is substituted with 0 to 5 R^{3c};
each R^{3a} is independently —OH, C₁₋₃ alkoxy, —O—(CH₂CH₂O)₁₋₄—C₁₋₄ alkyl, —O—(CH₂CH₂O)₁₋₄-heterocycloalkyl, C₁₋₃ haloalkoxy, —NR^{3a1}R^{3a2}, —O—C(O)C₁₋₄ alkyl, C₃₋₆ cycloalkyl, phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S;
each R^{3b} is independently C₁₋₄ alkyl, C₂₋₄ alkenyl, C₂₋₄ alkynyl, halo, C₁₋₄ haloalkyl, cyano, —OH, C₁₋₃ alkoxy, C₁₋₃ haloalkoxy, —NR^{3b1}R^{3b2}, —N(R^{3b3})C(O)R^{3b4}, phenyl, or heteroaryl having 5 to 6 ring members and 1 to 3 heteroatoms each independently N, O or S;
each R^{3c} is independently C₁₋₄ alkyl, C₁₋₄ haloalkyl, oxo, or C₃₋₆ cycloalkyl;
each R^{3a1}, R^{3a2}, R^{3b1}, R^{3b2}, and R^{3b3} is independently H or C₁₋₄ alkyl;
each R^{3b4} is C₁₋₄ alkyl, or C₁₋₄ haloalkyl;
R^{4a} is H or C₁₋₄ alkyl;
R^{4b} and R^{4c} are each independently H, C₁₋₈ alkyl, C₁₋₈ alkyl-OH, —NR^{4c1}R^{4c2}, C₁₋₄ alkyl-NR^{4c1}R^{4c2}, C₃₋₆ cycloalkyl, C₁₋₄ alkyl-C₃₋₆ cycloalkyl, heterocycloalkyl, C₁₋₄ alkyl-heterocycloalkyl, heteroaryl, or C₁₋₄ alkyl-heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O, or S;
alternatively, R^{4c} and R^{4a} together with the carbon and nitrogen to which each is attached combine to form a heterocycloalkyl having 4 to 6 ring members and 0 to 2 additional heteroatoms each independently N, O or S, wherein the heterocycloalkyl is substituted with 0 to 2 R^{4a1};
each R^{4c1} and R^{4c2} are independently C₁₋₄ alkyl or C₂₋₆ alkoxyalkyl;
each R^{4a1} is independently C₁₋₄ alkyl, —OH, C₁₋₄ alkyl-OH, C₁₋₄ alkoxy, halo, or —N(R^{4a2})S(O)₂—C₁₋₄ alkyl;
R^{4a2} is H or C₁₋₄ alkyl;
alternatively, two R^{4a1} groups on adjacent ring atoms combine to form a phenyl ring substituted with 0 to 2 R^{4a3};
each R^{4a3} is independently C₁₋₄ alkyl, —OH, C₁₋₄ alkyl-OH, C₁₋₄ alkoxy, or halo;
R^{5a} is H or C₁₋₄ alkyl;
R^{5b} and R^{5c} are each independently H, C₁₋₈ alkyl, C₁₋₈ alkyl-OH, C₂₋₆ alkoxyalkyl, C₁₋₈ haloalkyl, —C₁₋₄ alkyl-NR^{5b1}R^{5b2}, —C₁₋₃ alkyl-C(O)NR^{5b1}R^{5b2}, C₁₋₄ alkyl-N(R^{5b3})C(O)R^{5b4}, C₃₋₆ cycloalkyl, C₁₋₄ alkyl-C₃₋₆ cycloalkyl, heteroaryl, or C₁₋₄ alkyl-heteroaryl, wherein each heteroaryl has 5 to 6 ring members and 1 to 3 heteroatoms each independently N, O, or S, and wherein each cycloalkyl and heteroaryl is substituted with 0 to 3 R^{5b5};
each R^{5b1} and R^{5b2} are independently H, C₁₋₄ alkyl, C₁₋₄ haloalkyl, —C(O)C₁₋₄ alkyl, or —C(O)C₁₋₄ haloalkyl;
alternatively, R^{5b1} and R^{5b2} on the same nitrogen atom combine to form a heterocycloalkyl having 4 to 6 ring members and 0 to 2 additional heteroatoms each independently N, O or S, wherein the heterocycloalkyl is substituted with 0 to 3 R^{5b5};
each R^{5b3} is H or C₁₋₄ alkyl;
each R^{5b4} is a heteroaryl having 5 to 6 ring members and 1 to 3 heteroatoms each independently N, O or S, substituted with 0 to 3 R^{5b5};
each R^{5b5} is independently C₁₋₄ alkyl, halo, C₁₋₄ haloalkyl, —NH₂, —N(C₁₋₄alkyl)₂, or NH(C₁₋₄ alkyl);
X⁶ is C₂₋₅ alkylene;
R^{6a} is H, C₁₋₄ alkyl, C₁₋₄ deuteroalkyl, C₂₋₆ alkoxyalkyl, C₃₋₆ cycloalkyl, C₁₋₄ alkyl-C₃₋₆ cycloalkyl, heterocycloalkyl or C₁₋₄ alkyl-heterocycloalkyl, wherein the heterocycloalkyl has 4 to 6 ring members and 1 to 3 heteroatoms each independently N, O, or S;
R^{6b} is H or C₁₋₆ alkyl;
R^{6d} is H, C₁₋₄ alkyl, C₁₋₄ deuteroalkyl, —OH, or C₂₋₆ alkoxyalkyl;
R^{7a} is H or C₁₋₄ alkyl;
R^{7b} and R^{7c} are each independently H, C₁₋₈ alkyl, C₃₋₆ cycloalkyl, or C₁₋₄ alkyl-C₃₋₆ cycloalkyl;
R^{8a} is H, C₁₋₄ alkyl, C₁₋₄ deuteroalkyl, C₂₋₆ alkoxyalkyl, C₃₋₆ cycloalkyl or C₁₋₄ alkyl-C₃₋₆ cycloalkyl;
R^{8b}, R^{8d}, and R^{8c} are each independently H or C₁₋₄ alkyl;
alternatively R^{8b} and R^{8d} together with the carbons to which each is attached combine to form a C₃₋₆ cycloalkyl;
ring B is phenyl or heteroaryl having 5 to 12 ring members and 1 to 6 heteroatoms each independently N, O or S;
the subscript m8 is an integer from 0 to 5;
each R^{8f} is independently C₁₋₄ alkyl, C₂₋₄ alkenyl, C₂₋₄ alkynyl, C₁₋₄ alkoxy, C₂₋₈ alkoxyalkyl, halo, C₁₋₄ haloalkyl, C₁₋₄ haloalkoxy, cyano, —NR^{8f1}R^{8f2}, —C(O)NR^{8f1}R^{8f2}, —N(R^{8f1})C(O)R^{8f2}, C₃₋₆ cycloalkyl, —O—C₃₋₆ cycloalkyl, C₁₋₄ alkyl-C₃₋₆ cycloalkyl, —O—C₁₋₄ alkyl-C₃₋₆ cycloalkyl, heterocycloalkyl, C₁₋₄ alkyl-heterocycloalkyl, phenyl, —O-phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S, wherein each cycloalkyl, heterocycloalkyl, phenyl, and heteroaryl is substituted with 0 to 3 R^{8f3};
each R^{8f1} and R^{8f2} are independently H or C₁₋₄ alkyl;
each R^{8f3} is independently C₁₋₄ alkyl, —OH, C₁₋₄ alkoxy, —SH, —S—C₁₋₄ alkyl, halo, C₁₋₄ haloalkyl, C₁₋₄ haloalkoxy, —C(O)C₁₋₄ alkyl, —O—C₃₋₆ cycloalkyl, —O—C₁₋₄ alkyl-C₃₋₆ cycloalkyl, or heterocycloalkyl having 4 to 6 members and 0 to 2 additional heteroatoms each independently N, O or S;
X⁹ is C₁₋₃ alkylene substituted with R^{9b} and R^{9c};
R^{9a} is H or C₁₋₄ alkyl;
R^{9b} and R^{9c} are each independently H, C₁₋₆ alkyl, C₁₋₆ haloalkyl, C₁₋₆ alkyl-OH, C₂₋₆ alkoxyalkyl, C₃₋₆ cycloalkyl, C₁₋₄ alkyl-C₃₋₆ cycloalkyl, heteroaryl, or C₁₋₄ alkyl-heteroaryl, wherein each heteroaryl has 5 to 6 ring members and from 1 to 3 heteroatoms each independently N, O, or S, and each cycloalkyl and heteroaryl is independently substituted with 0 to 3 R^{9c1};
alternatively, R^{9b} and R^{9c} together with the carbon to which each is attached combine to form a C₃₋₄ cycloalkyl substituted with 0 to 2 R^{9c2}; or
alternatively, R^{9c} and R^{9a} together with the carbon and nitrogen to which each is attached combine to form a heterocycloalkyl having 4 to 6 members and 0 to 2 additional heteroatoms each independently N, O or S, wherein the heterocycloalkyl is substituted with 0 to 2 R^{9c2};
each R^{9c1} and R^{9c2} is independently C₁₋₄ alkyl, —OH, C₁₋₄ alkoxy, halo, C₁₋₄ haloalkyl, or C₁₋₄ haloalkoxy; and
ring A comprises 15 to 17 ring atoms;
or a pharmaceutically acceptable salt thereof.

2. The compound of claim 1, or the pharmaceutically acceptable salt thereof, wherein R³ is
(a) C₁₋₆ alkyl, C₂₋₆ alkynyl, or C₁₋₆ haloalkyl, each substituted with 0 to 5 R^{3a};
(b) C₃₋₁₂ cycloalkyl substituted with 0 to 5 R^{3b}; or
(c) heterocycloalkyl having 3 to 6 ring members and 1 to 3 heteroatoms each independently N, O, or S, wherein the heterocycloalkyl is substituted with 0 to 5 R^{3c};
each R^{3a} is independently —OH, C₁₋₃ alkoxy, —O—(CH₂CH₂O)₁₋₃—C₁₋₄ alkyl, —O—(CH₂CH₂O)₁₋₂-heterocycloalkyl, C₁₋₃ haloalkoxy, —NR^{3a1}R^{3a2}, —O—C(O)C₁₋₄ alkyl, C₃₋₆ cycloalkyl, phenyl, or heteroaryl, wherein each heterocycloalkyl has 4 to 6 ring members and 1 to 3 heteroatoms each independently N, O, or S having 5 to 6 ring members and 1 to 3 heteroatoms each independently N, O or S;
each R^{3b} is independently C₁₋₄ alkyl, C₂₋₄ alkynyl, halo, C₁₋₄ haloalkyl, cyano, —N(R^{3b3})C(O)R^{3b4}, phenyl, or heteroaryl having 5 to 6 ring members and 1 to 3 heteroatoms each independently N, O or S;
each R^{3c} is independently C₁₋₄ alkyl, C₁₋₄ haloalkyl, oxo, or C₃₋₆ cycloalkyl;
each R^{3a1}, R^{3a2}, and R^{3b3} is independently H or C₁₋₄ alkyl; and
each R^{3b4} is C₁₋₄ alkyl.

3. (canceled)

4. The compound of claim 1, or the pharmaceutically acceptable salt thereof, wherein R³ is

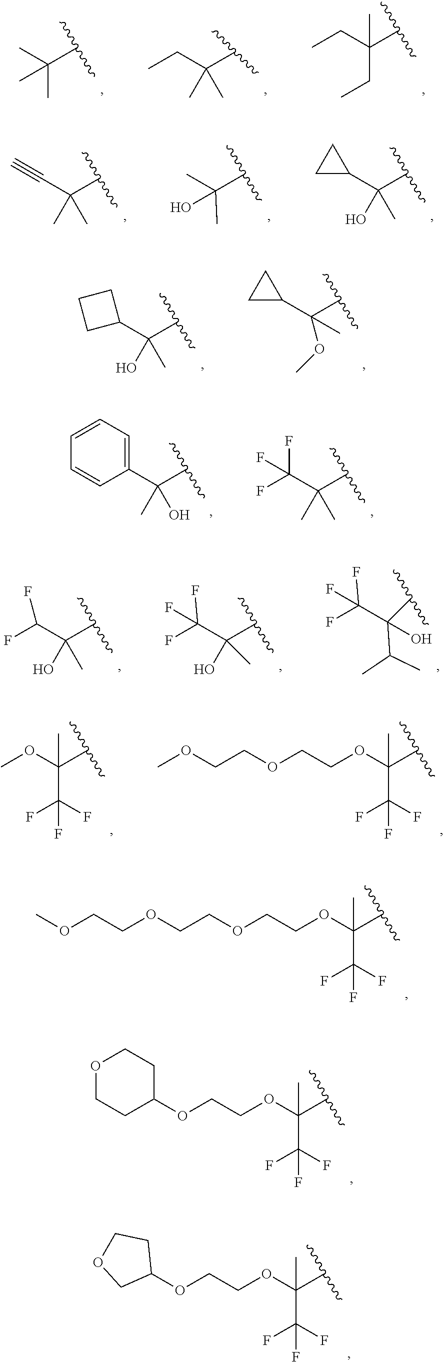

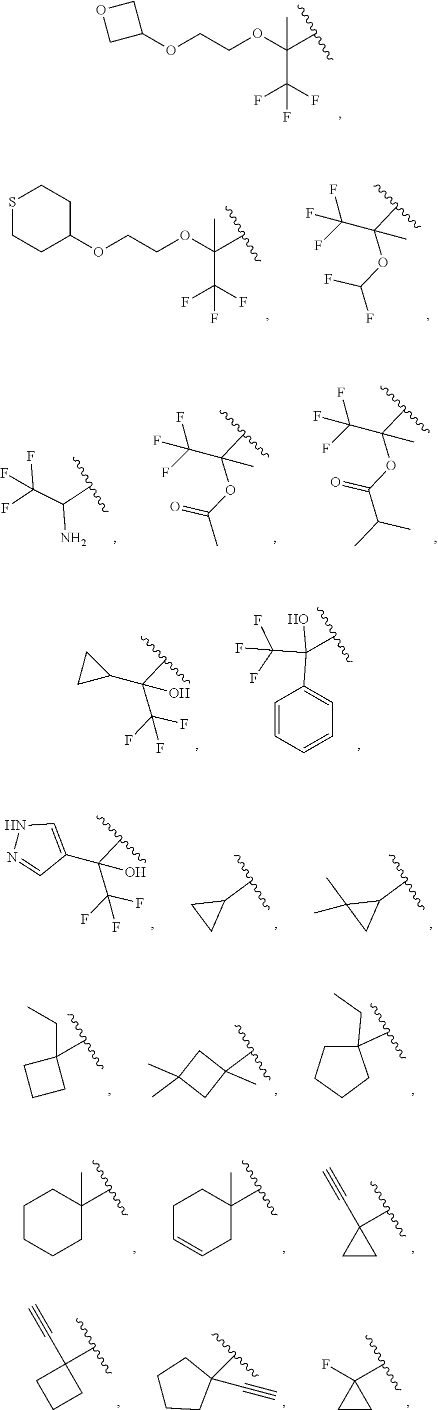

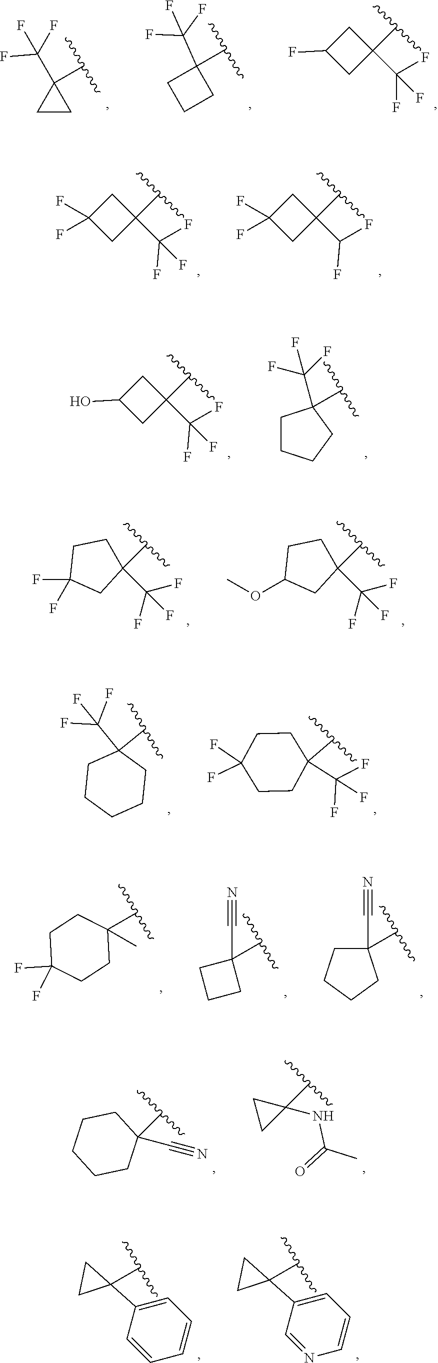

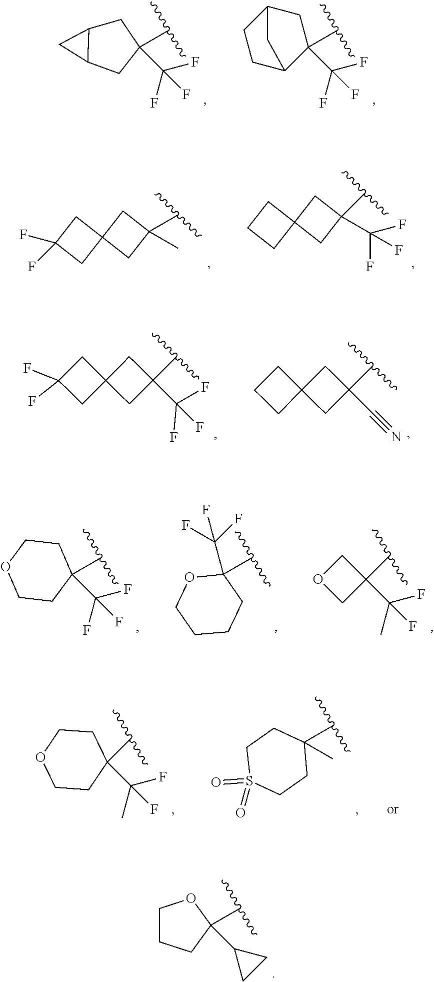

5.-6. (canceled)

7. The compound of claim 1, or the pharmaceutically acceptable salt thereof, wherein
R^{4a} is H or C₁₋₄ alkyl;
R^{4b} and R^{4c} are each independently H, C₁₋₈ alkyl, or C₁₋₄ alkyl-NR^{4c1}R^{4c2};
alternatively R^{4c} and R^{4a} together with the carbon and nitrogen to which each is attached combine to form a heterocycloalkyl having 4 to 6 ring members and 0 to 2 additional heteroatoms each independently N, O or S, wherein the heterocycloalkyl is substituted with 0 to 2 R^{4a1};
each R^{4c1} and R^{4c2} are independently C₁₋₄ alkyl;
each R^{4a1} is independently —OH, or halo;
alternatively, two R^{4a1} groups on adjacent ring atoms combine to form a phenyl ring substituted with 0 to 2 R^{4a3}; and
each R^{4a3} is —OH.

8. The compound of claim 1, or the pharmaceutically acceptable salt thereof, wherein
R^{4a} is H or methyl;
R^{4b} is H;
R^{4c} is methyl, ethyl, isopropyl, tert-butyl, 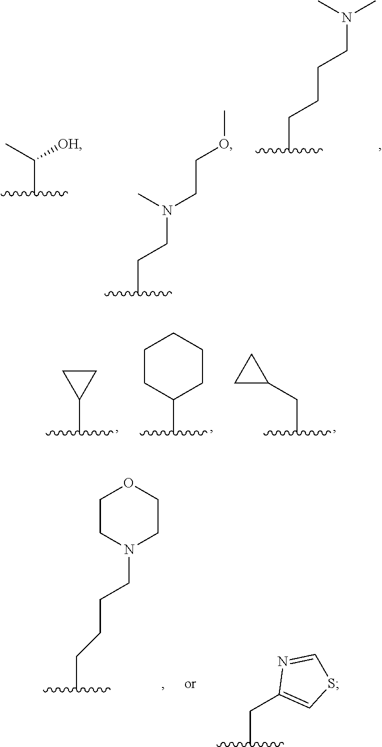
alternatively R^{4c} and R^{4a} together with the carbon and nitrogen to which each is attached combine to form a heterocycloalkyl having 4 to 6 ring members and 0 to 1 additional oxygen, wherein the heterocycloalkyl is substituted with 0 to 2 R^{4a1}; and
each R^{4a1} is independently methyl, —OH, methoxy, fluoro, or —N(H)S(O)₂CH₃;
alternatively, two R^{4a1} groups on adjacent ring atoms combine to form a phenyl ring substituted with 0 to 2 —OH.

9. (canceled)

10. The compound of claim 1, or the pharmaceutically acceptable salt thereof, wherein
R^{5a} is H;
R^{5b} and R^{5c} are each independently H, C₁₋₈ alkyl, C₁₋₈ alkyl-OH, C₂₋₆ alkoxyalkyl, C₁₋₈ haloalkyl, —C₁₋₄ alkyl-NR^{5b1}R^{5b2}, —C₁₋₃ alkyl-C(O)NR^{5b1}R^{5b2}, —C₁₋₄ alkyl-N(R^{5b3})C(O)R^{5b4}, C₃₋₆ cycloalkyl, or C₁₋₄ alkyl-C₃₋₆ cycloalkyl, wherein each cycloalkyl is substituted with 0 to 3 R^{5b5};
each R^{5b1} and R^{5b2} are independently H, C₁₋₄ alkyl, C₁₋₄ haloalkyl, —C(O)C₁₋₄ alkyl, or —C(O)C₁₋₄ haloalkyl, provided that no more than one of R^{5b1} and R^{5b2} is H;
alternatively, R^{5b1} and R^{5b2} on the same nitrogen atom combine to form a heterocycloalkyl having 6 ring members and 0 to 1 additional oxygen ring members, wherein the heterocycloalkyl is substituted with 0 to 2 R^{5b5};
each R^{5b3} is H or C₁₋₄ alkyl;
each R^{5b4} is a heteroaryl having 5 to 6 ring members and 1 to 3 heteroatoms each independently N, O or S substituted with 0 to 1 R^{5b5}; and
each R^{5b5} is independently C₁₋₄ alkyl, halo, C₁₋₄ haloalkyl, or NH(CH₃).

11.-12. (canceled)

13. The compound of claim 1, or the pharmaceutically acceptable salt thereof, wherein
R^{5a} is H;
R^{5b} is H; and
R^{5c} is H, methyl, ethyl, n-propyl, isopropyl, n-butyl, sec-butyl, isobutyl, tert-butyl,

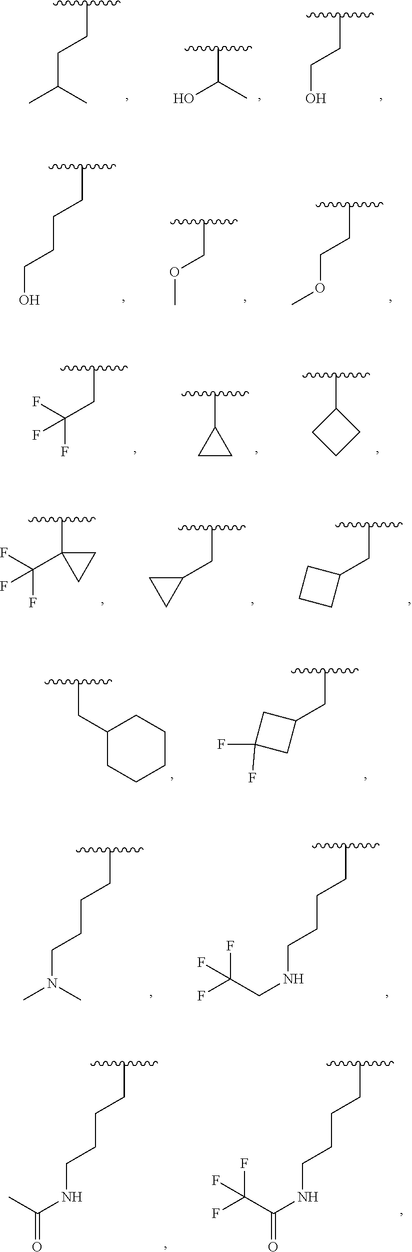

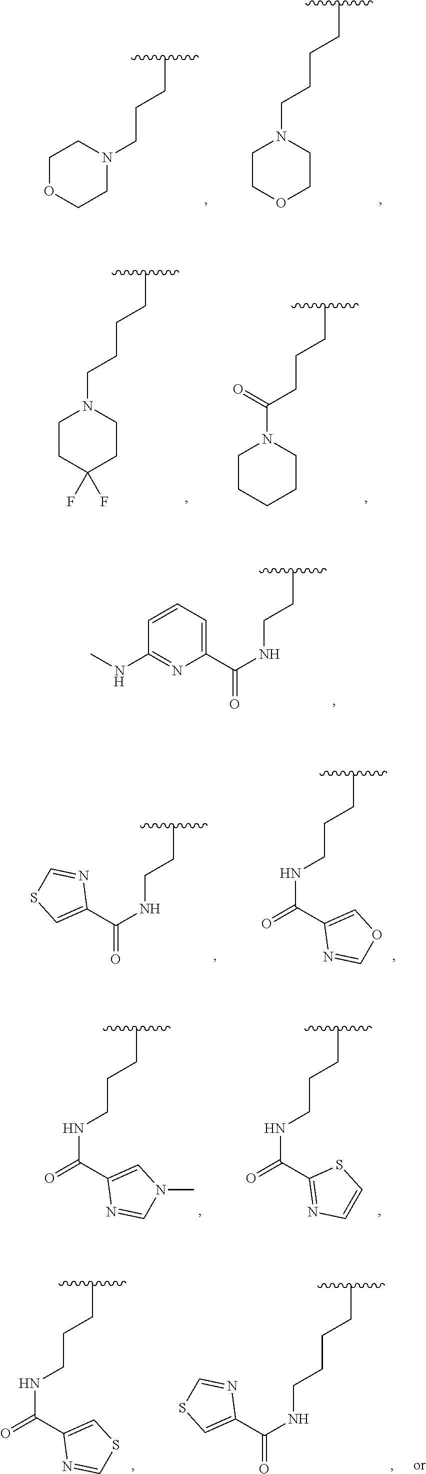

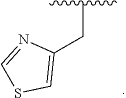 [SMILES: *Cc1cscn1]

14-16. (canceled)

17. The compound of claim 1, or the pharmaceutically acceptable salt thereof, wherein
R^{6a} is H, methyl, ethyl, n-propyl, isopropyl, n-butyl, isobutyl, —CD₃, 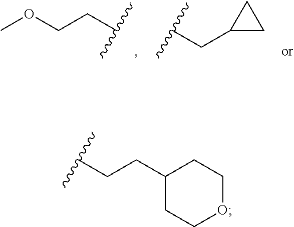
R^{6b} is H; and
R^{6d} is H, methyl, ethyl, n-propyl, isopropyl, —CD₃, or

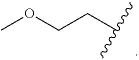 [SMILES: *CCOC]

18.-22. (canceled)

23. The compound of claim 1, or the pharmaceutically acceptable salt thereof, wherein
R^{7a} is H;
R^{7b} is H; and
R^{7c} is isobutyl,

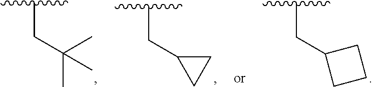

24. (canceled)

25. The compound of claim 1, or the pharmaceutically acceptable salt thereof, having the structure of Formula Ia:

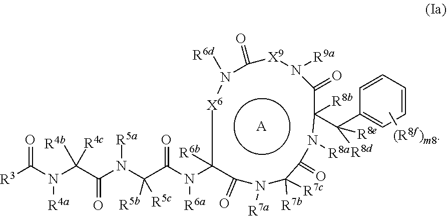

26.-31. (canceled)

32. The compound of claim 1, or the pharmaceutically acceptable salt thereof, wherein
R^{8a} is C₁₋₄ alkyl, C₁₋₄ deuteroalkyl, C₂₋₆ alkoxyalkyl, or C₁₋₄ alkyl-C₃₋₆ cycloalkyl;
R^{8b}, R^{8d}, and R^{8e} are each independently H;
alternatively R^{8b} and R^{8d} together with the carbons to which each is attached combine to form a C₃₋₆ cycloalkyl;
the subscript m8 is an integer from 0 to 5;
each R^{8f} is independently C₁₋₄ alkyl, C₂₋₄ alkenyl, C₂₋₄ alkynyl, C₁₋₄ alkoxy, C₂₋₈ alkoxyalkyl, halo, C₁₋₄ haloalkyl, C₁₋₄ haloalkoxy, cyano, —NR^{8f1}R^{8f2}, —C(O)NR^{8f1}R^{8f2}, —N(R^{8f1})C(O)R^{8f2}, C₃₋₆ cycloalkyl, —O—C₃₋₆ cycloalkyl, C₁₋₄ alkyl-C₃₋₆ cycloalkyl, —O—C₁₋₄ alkyl-C₃₋₆ cycloalkyl, heterocycloalkyl, —C₁₋₄ alkyl-heterocycloalkyl, phenyl, —O-phenyl, or heteroaryl, wherein each heterocycloalkyl has 4 to 6 ring members and 1 to 3 heteroatoms each independently N, O, or S, and each heteroaryl has 5 to 6 ring members and 1 to 3 heteroatoms each independently N, O or S, wherein each cycloalkyl, heterocycloalkyl, phenyl, and heteroaryl is substituted with 0 to 3 R^{8f3};
each R^{8f1} and R^{8f2} are independently H or C₁₋₄ alkyl; and
each R^{8f3} is independently C₁₋₄ alkyl, —OH, C₁₋₄ alkoxy, halo, C₁₋₄ haloalkyl, C₁₋₄ haloalkoxy, —C(O)C₁₋₄ alkyl, or heterocycloalkyl having 4 to 6 members and 0 to 2 additional heteroatoms each independently N, O or S.

33.-34. (canceled)

35. The compound of claim 1, or the pharmaceutically acceptable salt thereof, wherein
R^{8a} is methyl, ethyl, n-propyl, n-butyl, —CD₃, or 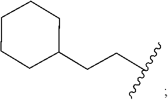 [SMILES: *CCC1CCCCC1]
and
R^{8b}, R^{8d} and R^{8c} are each H.

36. The compound of claim 1, or the pharmaceutically acceptable salt thereof, wherein
m8 is 0, 1, 2, or 3; and
each R^{8f} is independently methyl, ethynyl, methoxy, fluoro, chloro, bromo, iodo,

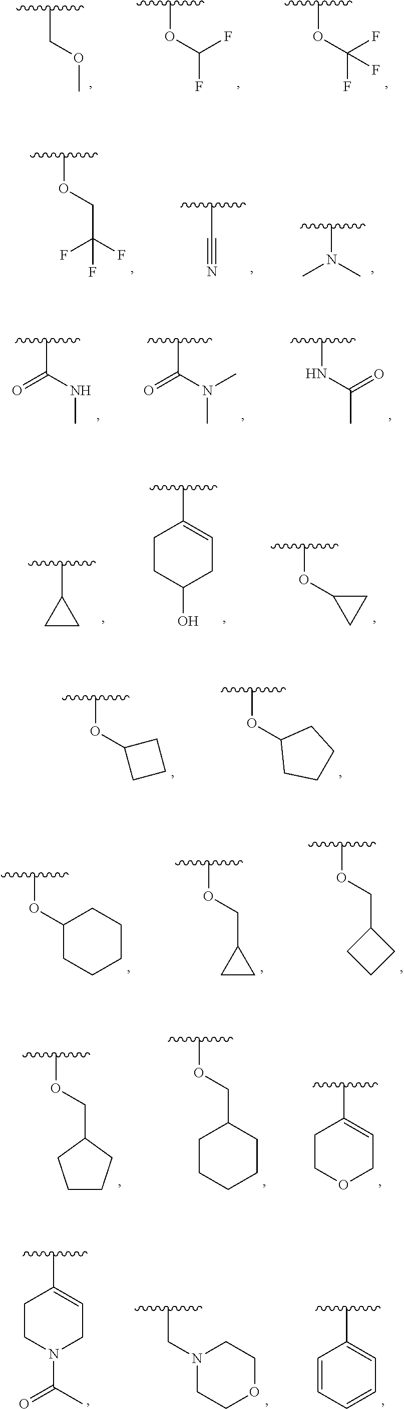

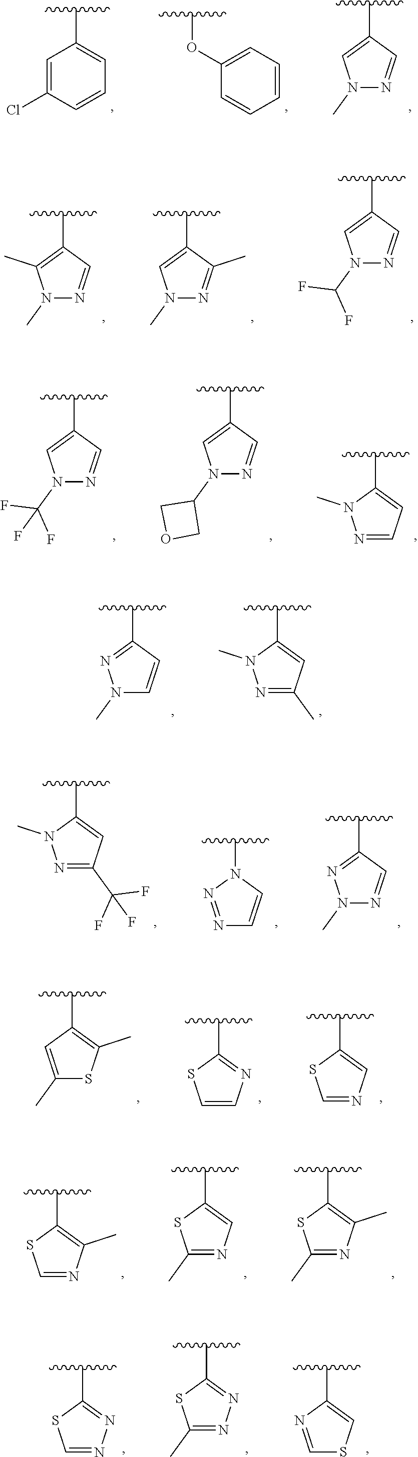

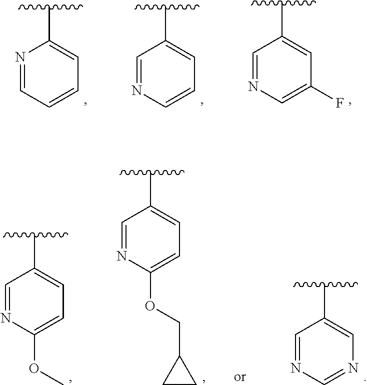

37. (canceled)

38. The compound of claim 1, or the pharmaceutically acceptable salt thereof, wherein the subscript m8 is 2.

39.-43. (canceled)

44. The compound of claim 1, or the pharmaceutically acceptable salt thereof, wherein
R^{9a} is H or methyl;
R^{9b} is H, methyl, or ethyl; and
R^{9c} is H, methyl, ethyl, n-propyl, sec-butyl, 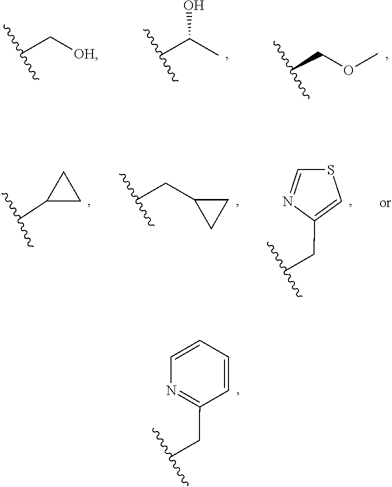
alternatively, R^{9b} and R^{9c} together with the carbon to which they are attached combine to form a C₃₋₄ cycloalkyl substituted with 0 to 2 fluoro groups;
alternatively, R^{9c} and R^{9a} together with the carbon and nitrogen to which each is attached combine to form a heterocycloalkyl having 4- to 6-ring members and 0 additional heteroatoms, the heterocycloalkyl is substituted with 0 or 1 fluoro or —OH groups.

45.-47. (canceled)

48. The compound of claim 1, or the pharmaceutically acceptable salt thereof, having the structure of Formula Ib:

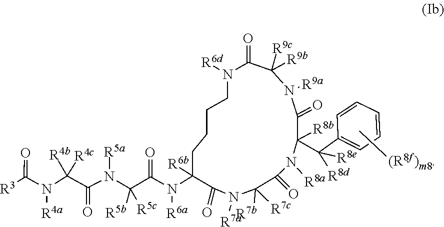

49.-51. (canceled)

52. The compound of claim 1, or the pharmaceutically acceptable salt thereof, wherein
R³ is 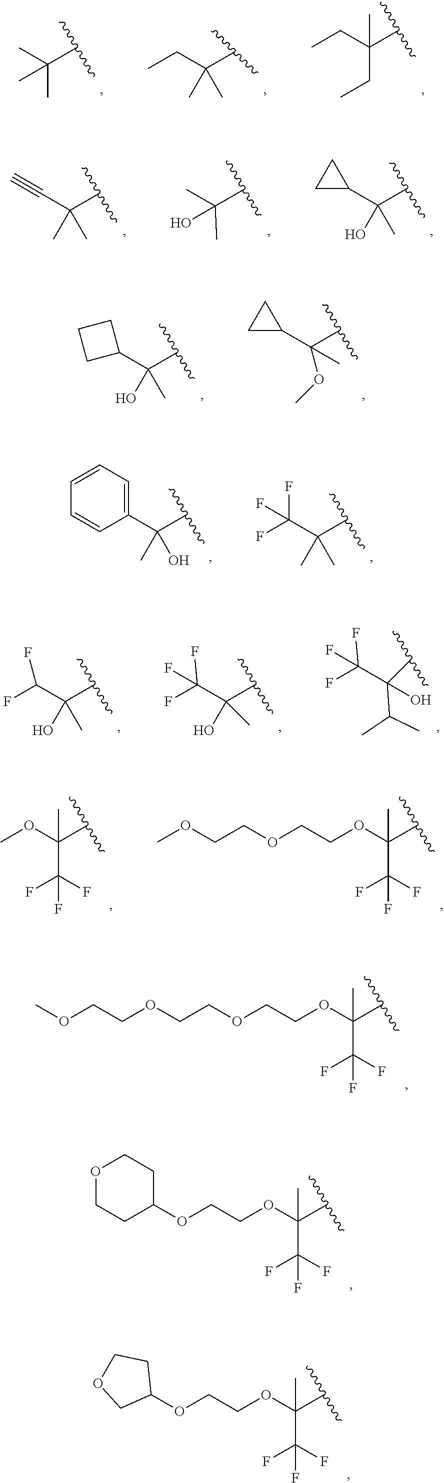 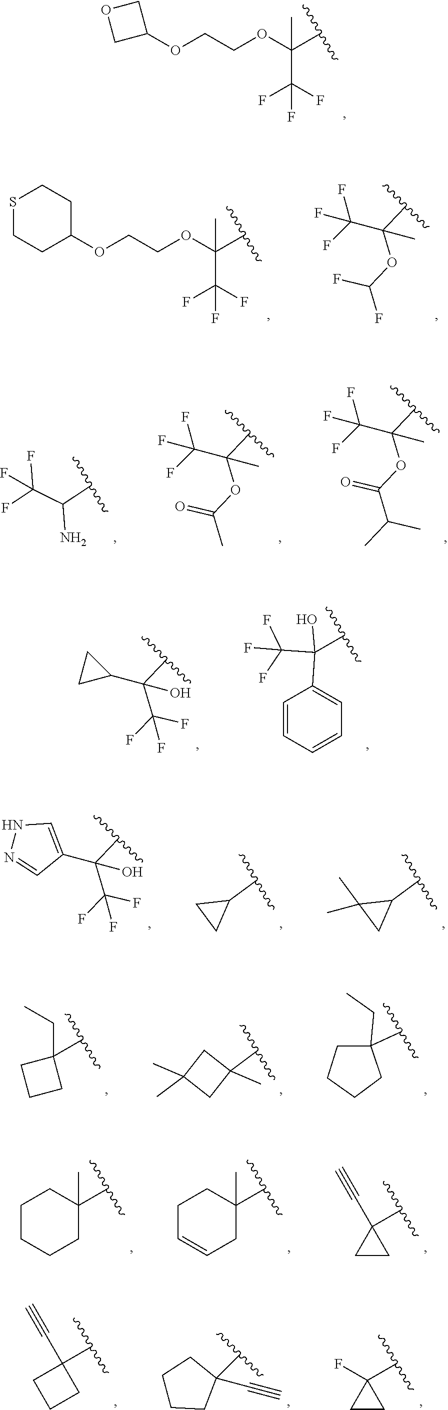 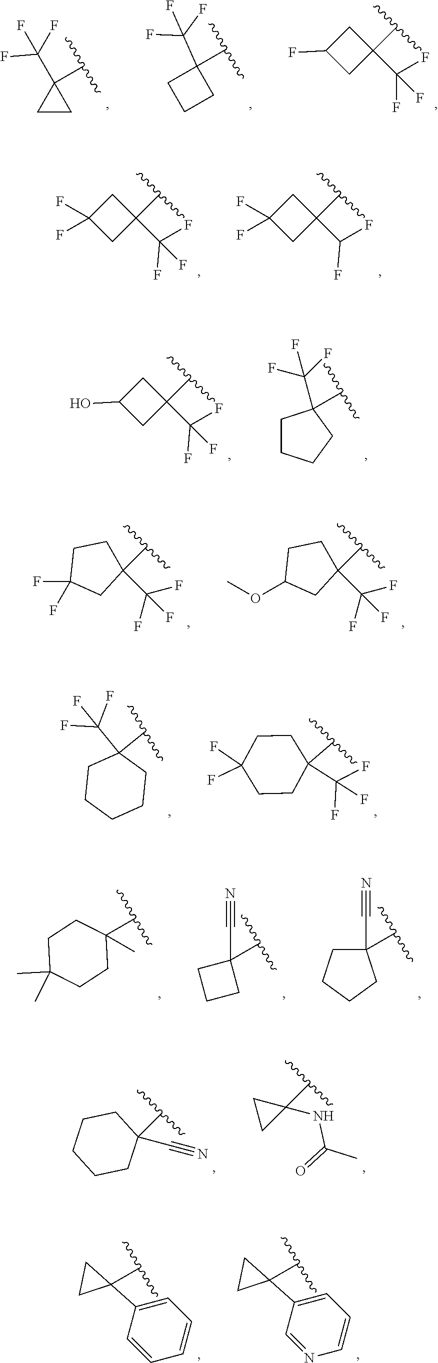 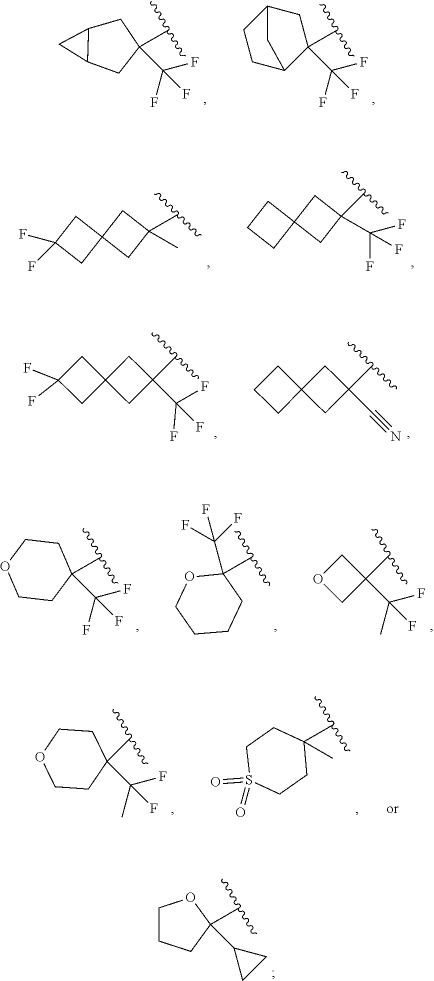
R^{4a} is H or methyl;
R^{4b} is H;
R^{4c} is methyl, ethyl, isopropyl, tert-butyl, 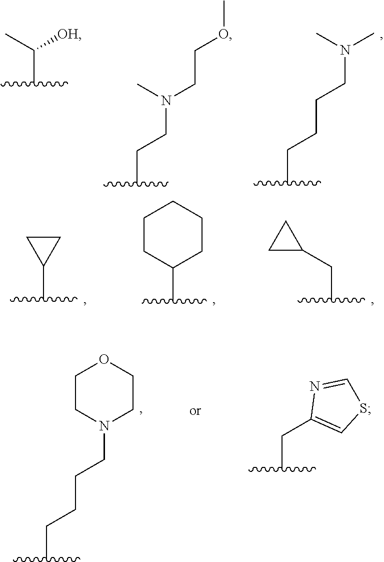
alternatively R^{4c} and R^{4a} together with the carbon and nitrogen to which each is attached combine to form a heterocycloalkyl having 4 to 6 ring members and 0 to 1 additional oxygen, wherein the heterocycloalkyl is substituted with 0 to 2 R^{4a1};
each R^{4a1} is independently methyl, —OH, methoxy, fluoro, or —N(H)S(O)₂CH₃;
alternatively, two R^{4a1} groups on adjacent ring atoms combine to form a phenyl ring substituted with 0 to 2 —OH;
R^{5a} is H;
R^{5b} is H;
R^{5c} is H, methyl, ethyl, n-propyl, isopropyl, n-butyl, sec-butyl, isobutyl, tert-butyl, 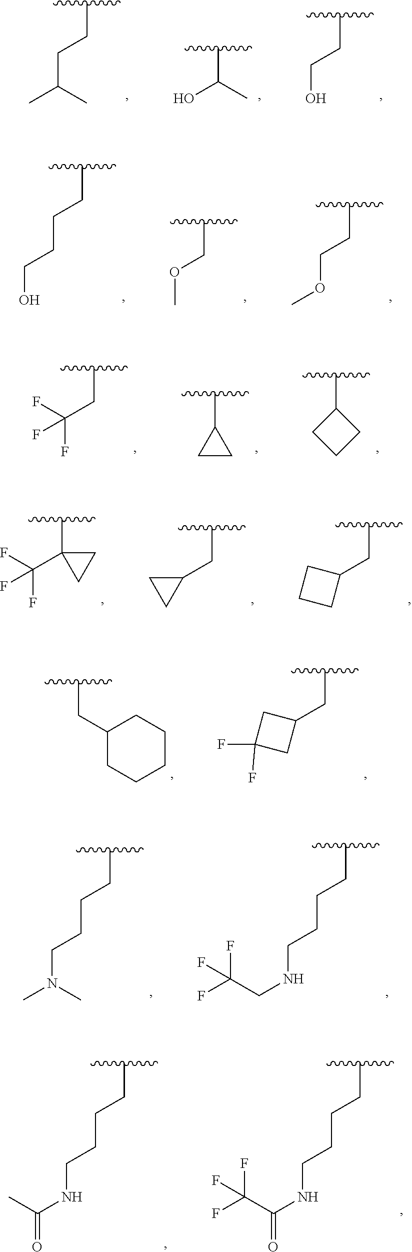 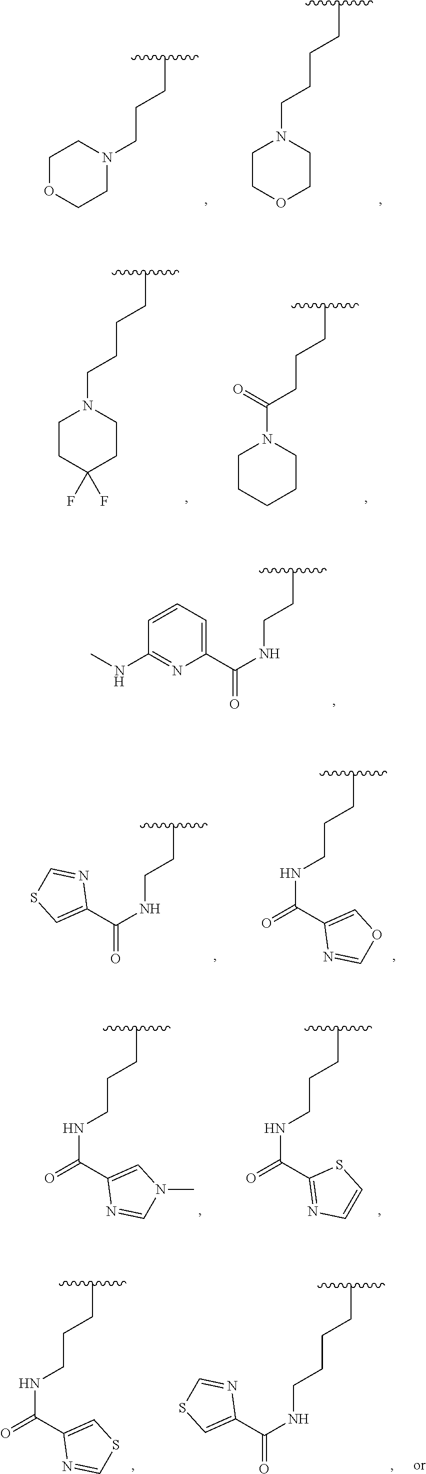 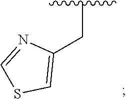 [SMILES: *Cc1cscn1]
X⁶ is 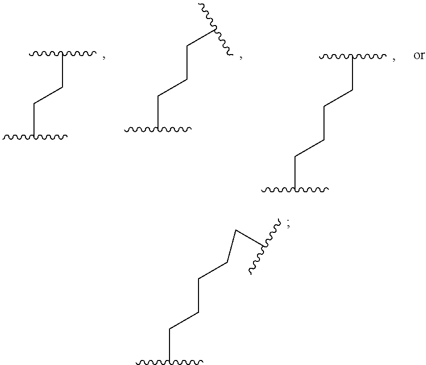
R^{6a} is H, methyl, ethyl, n-propyl, isopropyl, n-butyl, isobutyl, —CD₃, 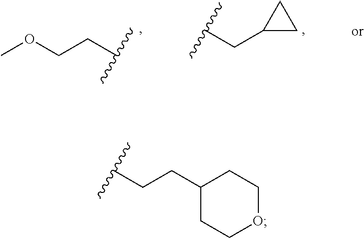
R^{6b} is H;
R^{6d} is H, methyl, ethyl, n-propyl, isopropyl, —CD₃, or 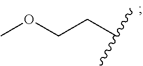 [SMILES: *CCOC]
R^{7a} is H;
R^{7b} is H;
R^{7c} is isobutyl, 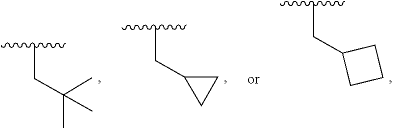
R^{8a} is methyl, ethyl, n-propyl, n-butyl, —CD₃, 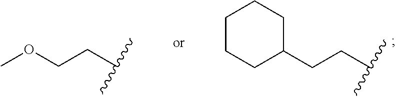
R^{8b}, R^{8d} and R^{8e} are each H;
alternatively, R^{8b} and R^{8d} together with the carbons to which each is attached combine to form a cyclopropyl;
m8 is 0, 1, 2, or 3;
each R^{8f} is independently methyl, ethynyl, methoxy, fluoro, chloro, bromo, iodo, 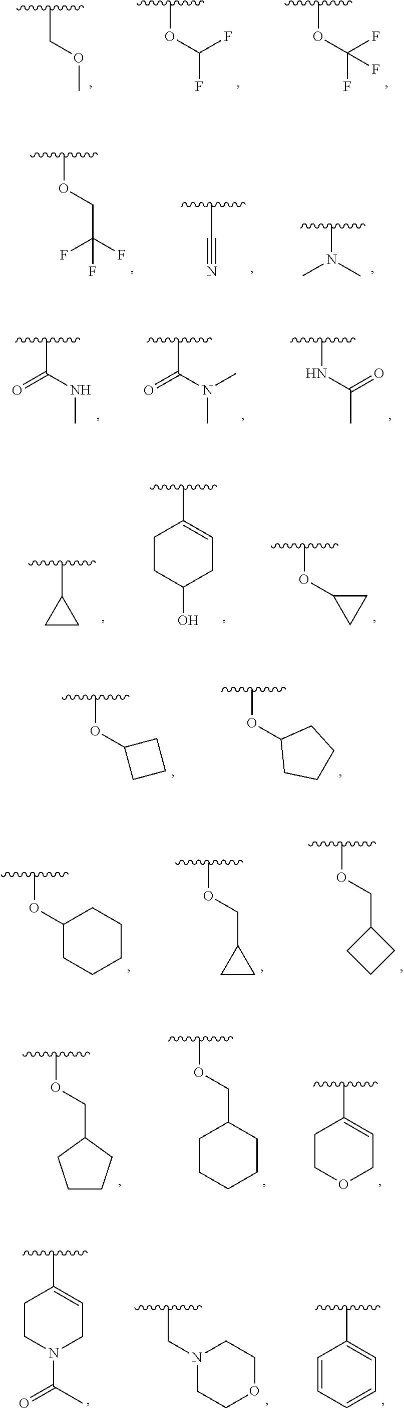 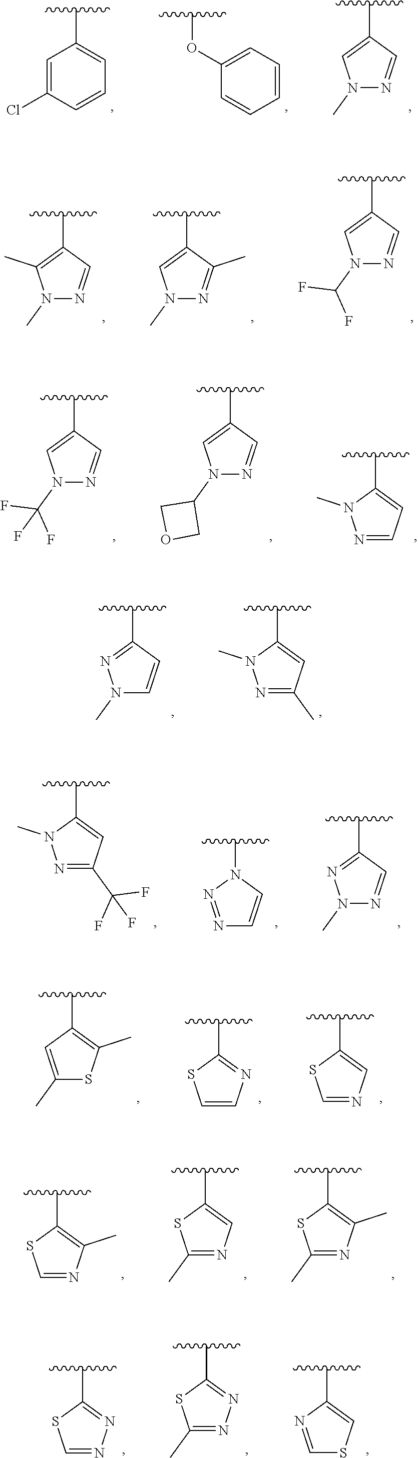 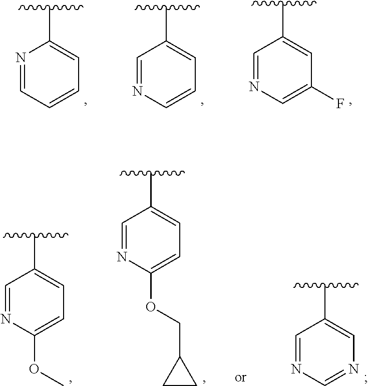
X⁹ is 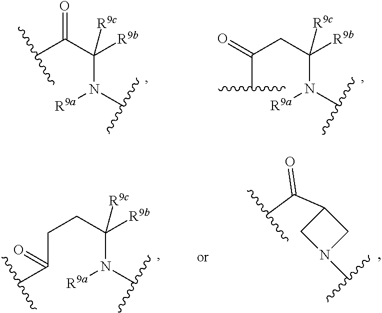
R^{9a} is H or methyl;
R^{9b} is H, methyl, or ethyl; and
R^{9c} is H, methyl, ethyl, n-propyl, sec-butyl, 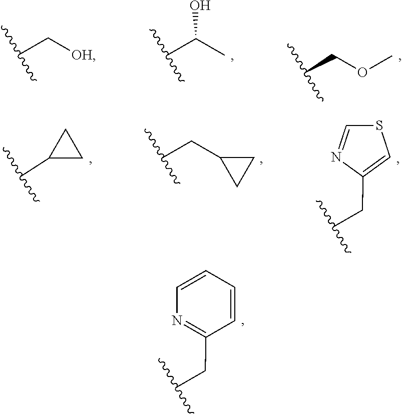
alternatively, R^{9b} and R^{9c} together with the carbon to which they are attached combine to form a C₃₋₄ cycloalkyl substituted with 0 to 2 fluoro groups;
alternatively, R^{9c} and R^{9a} together with the carbon and nitrogen to which each is attached combine to form a heterocycloalkyl having 4- to 6-ring members and 0 additional heteroatoms, the heterocycloalkyl is substituted with 0 or 1 fluoro or —OH groups.

53. (canceled)

54. The compound of claim 1, or the pharmaceutically acceptable salt thereof, having the structure of Formula Ic:

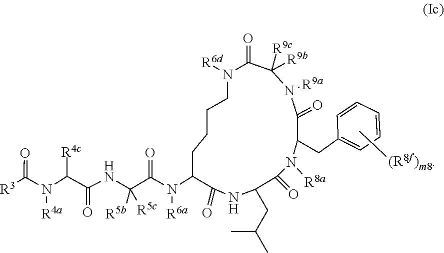

55. (canceled)

56. The compound of claim 1 having the structure of any one of Examples 1-693.

57. A pharmaceutical composition comprising a compound of claim 1 and a pharmaceutically acceptable excipient.

58. A method of treating a cancer mediated at least in part by one or more cyclins, the method comprising administering to a subject in need thereof, a therapeutically effective amount of a compound of claim 1, thereby treating the disorder or condition.

59.-60. (canceled)
